(19) United States
(12) Patent Application Publication
(10) Pub. No.: US 2024/0327393 A1
(43) Pub. Date: Oct. 3, 2024

(54) FERROPTOSIS MODULATORS, PREPARATIONS, AND USES THEREOF

(71) Applicant: SIRONAX LTD., Grand Cayman (KY)

(72) Inventors: Jianguang HAN, Beijing (CN); Zhiyuan ZHANG, Beijing (CN); Weijie HOU, Beijing (CN); Yimin JIANG, Beijing (CN); Yanping XU, Beijing (CN)

(21) Appl. No.: 18/574,309

(22) PCT Filed: Jul. 8, 2022
(86) PCT No.: PCT/CN2022/104559

§ 371 Date: Dec. 27, 2023

(30) Foreign Application Priority Data
Jul. 9, 2021 (WO) PCT/CN2021/105449

(51) Int. Cl. C07D 413/12; A61K 31/366; A61K 31/4035; A61K 31/416; A61K 31/4184; A61K 31/423; A61K 31/428; A61K 31/437; A61K 31/4439; A61K 31/454; A61K 31/4545; A61K 31/4709; A61K 31/472; A61K 31/496; A61K 31/498; A61K 31/506; A61K 31/517; A61K 31/5355; A61K 31/536; A61K 31/538; A61K 31/5383; A61K 31/5415; A61K 31/5513; A61K 31/553; C07D 209/46; C07D 217/04; C07D 217/06; C07D 263/58; C07D 277/82; C07D 279/16; C07D 311/18; C07D 401/12; C07D 401/14; C07D 403/12; C07D 405/12; C07D 405/14; C07D 409/12; C07D 413/14; C07D 471/04; C07D 487/04; C07D 491/08; C07D 498/04; C07D 498/08

(52) U.S. Cl. CPC C07D 413/12; A61K 31/366; A61K 31/4035; A61K 31/416; A61K 31/4184; A61K 31/423; A61K 31/428; A61K 31/437; A61K 31/4439; A61K 31/454; A61K 31/4545; A61K 31/4709; A61K 31/472; A61K 31/496; A61K 31/498; A61K 31/506; A61K 31/517; A61K 31/5355; A61K 31/536; A61K 31/538; A61K 31/5383; A61K 31/5415; A61K 31/5513; A61K 31/553; C07D 209/46; C07D 217/04; C07D 217/06; C07D 263/58; C07D 277/82; C07D 279/16; C07D 311/18; C07D 401/12; C07D 401/14; C07D 403/12; C07D 405/12; C07D 405/14; C07D 409/12; C07D 413/14; C07D 471/04; C07D 487/04; C07D 491/08; C07D 498/04; C07D 498/08

(57) ABSTRACT

Provided are compounds of Formula I, compositions comprising the same, and methods of using the same, including use in treating various diseases and conditions, e.g., those involving ferroptosis.

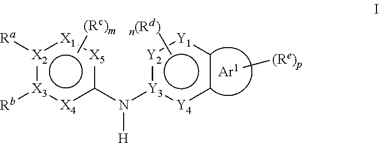

### RELATED APPLICATION

This application claims priority to International Application No. PCT/CN2021/105449, filed on Jul. 9, 2021, the content of which is incorporated by reference in its entirety.

### FIELD OF THE DISCLOSURE

The present disclosure relates to compounds that modulate ferroptosis, compositions comprising the compounds, methods of preparing the compounds, and methods of using the compounds to treat various diseases or conditions, e.g., those involving ferroptosis.

### BACKGROUND OF THE DISCLOSURE

Ferroptosis is a type of programmed cell death dependent on iron and characterized by the accumulation of lipid peroxides, and is genetically and biochemically distinct from other forms of regulated cell death such as apoptosis, autophagy, and necrosis. (Dixon et al., Cell 149, 1060-1072 (2012).) Certain mechanisms of ferroptosis and key contributors to ferroptotic cell death is reviewed and illustrated by Conrad et al., Nature Chemical Biology, vol. 15, Dec. 2019, 1137-1147.

Ferroptosis has been implicated in a number of diseases or conditions, including neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. For example, inactivation of the ferroptosis regulator Gpx4 triggers acute renal failure in mice. (Angeli, et al., Nature Cell Biology, published online Nov. 17, 2014; DOI: 10.1038/ncb3064.) Ferroptosis has been considered to be implicated in several pathophysiological contexts such as tumor suppression, antiviral immunity, neurodegeneration, and ischemia/reperfusion injury (IRI). (Angeli et al., Trends in Pharmacological Sciences, May 2017, Vol. 38, No. 5, 489-498 (2017).)

Certain compounds that modulate ferroptosis are disclosed by Skouta et al., J. Am. Chem. Soc., dx.doi.org/10.1021/ja411006a; and in WO2013152039A1, WO2015084749A1, WO2019154795A1, WO2016075137A1, WO2020185738A1, and PCT/CN2021/078601.

### SUMMARY OF THE DISCLOSURE

One aspect of this disclosure provides a compound selected from compounds of the Formulae disclosed herein, a tautomer thereof, a solvate or stereoisomer of the compound or the tautomer, or a pharmaceutically acceptable salt of the foregoing, which can be employed in the treatment of various diseases or conditions, such as diseases or conditions involving ferroptosis. For example, disclosed herein is a compound of the following structural Formula I:

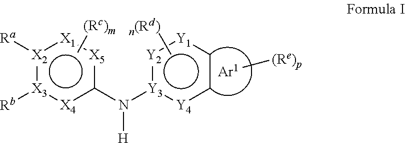

a tautomer thereof, a solvate or stereoisomer of the compound or the tautomer, or a pharmaceutically acceptable salt of the foregoing, wherein:
Ar¹ is aryl, cycloalkyl, heteroaryl, or 5- to 7-membered heterocyclyl;
X₁ is C, N, or S;
X₂, X₃, X₅, Y₁, Y₂, Y₃, and Y₄ are each independently C or N;
X₄ is C or N, but when X₁ is S, X₄ is nothing;
R^{a} is selected from H, halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl;
R^{b} is selected from H, halogen, CN, optionally substituted heteroatom, optionally substituted alkyl, optionally substituted cycloalkyl, and optionally substituted heterocyclyl;
R^{a} and R^{b} may join together to form an optionally substituted 3- to 10-membered carbocyclic, heterocyclic, aromatic, or heteroaromatic ring;
R^{c}, for each occurrence, is independently selected from halogen, CN, optionally substituted heteroatom, and optionally substituted alkyl;
R^{d}, for each occurrence, is independently selected from halogen, CN, optionally substituted heteroatom, and optionally substituted alkyl;
R^{e}, for each occurrence, is independently selected from halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl; and
m, n, and p are each an integer independently selected from 0, 1, 2, and 3;
provided that:
(1)

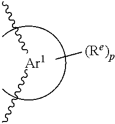

is not

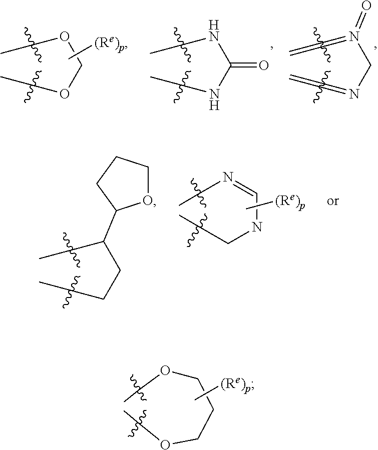

(2) when

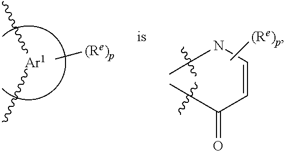

R^{e} is not —C(═O)OH or —C(═O)O(C₁-C₃ alkyl);
(3) when

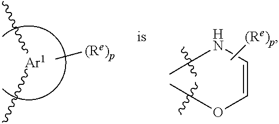

R^{a} is not OH; and
(4) when Ar¹ is a 5- to 6-membered cycloalkyl, 5- to 6-membered heteroaryl, or 5- to 7-membered heterocyclyl, (i) R^{a} is selected from optionally substituted cycloalkyl, optionally substituted heterocyclyl, optionally substituted aryl, and optionally substituted heteroaryl, (ii) R^{a} is selected from H, halogen, CN, optionally substituted heteroatom, optionally substituted alkyl, optionally substituted alkenyl, optionally substituted alkynyl, provided that p is an integer selected from 1, 2, and 3 and that at least one of the R^{e} is ═O, or (iii) R^{a} and R^{b} join together to form an optionally substituted 3- to 10-membered carbocyclic, heterocyclic, aromatic, or heteroaromatic ring, provided that p is an integer selected from 1, 2, and 3 and that at least one of the R^{e} is ═O.

In one aspect of the disclosure, the compounds of the Formulae disclosed herein are selected from Compounds 1 to 659 shown below, a tautomer thereof, a solvate or stereoisomer of the compound or the tautomer, or a pharmaceutically acceptable salt of the foregoing.

In some embodiments, the disclosure provides pharmaceutical compositions comprising a compound of the Formulae disclosed herein, a tautomer thereof, a solvate or stereoisomer of the compound or the tautomer, or a pharmaceutically acceptable salt of the foregoing, and a pharmaceutically acceptable carrier. In some embodiments, the pharmaceutical compositions may comprise a compound selected from Compounds 1 to 659 shown below, a tautomer thereof, a solvate or stereoisomer of the compound or the tautomer, or a pharmaceutically acceptable salt of the foregoing, and a pharmaceutically acceptable carrier. These compositions may further comprise an additional active pharmaceutical agent.

Another aspect of the disclosure provides methods of treating a disease or condition, comprising administering to a subject in need thereof, a therapeutically effective amount of a compound of the Formulae disclosed herein, a tautomer thereof, a solvate or stereoisomer of the compound or the tautomer, or a pharmaceutically acceptable salt of the foregoing, or a pharmaceutical composition comprising any of the foregoing, wherein the disease or condition is selected from neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure.

A further aspect of the disclosure provides methods of treating a disease or condition involving ferroptosis, comprising administering to a subject in need thereof, a therapeutically effective amount of a compound of the Formulae disclosed herein, a tautomer thereof, a solvate or stereoisomer of the compound or the tautomer, or a pharmaceutically acceptable salt of the foregoing, or a pharmaceutical composition comprising any of the foregoing.

In some embodiments, the methods of treatment comprise administering to a subject in need thereof, a compound selected from Compounds 1 to 659 shown below, a tautomer thereof, a solvate or stereoisomer of the compound or the tautomer, or a pharmaceutically acceptable salt of the foregoing, or a pharmaceutical composition comprising any of the foregoing.

In some embodiments, the methods of treatment comprise administration of an additional active pharmaceutical agent to the subject in need thereof, either in the same pharmaceutical composition as a compound of the Formulae disclosed herein, a tautomer thereof, a solvate or stereoisomer of the compound or the tautomer, or a pharmaceutically acceptable salt of the foregoing, or in a separate composition. In some embodiments, the methods of treatment comprise administering a compound selected from Compounds 1 to 659 shown below, a tautomer thereof, a solvate or stereoisomer of the compound or the tautomer, or a pharmaceutically acceptable salt of the foregoing with an additional active pharmaceutical agent either in the same pharmaceutical composition or in a separate composition. When administered as a separate composition, the additional therapeutic agent may be administered prior to, at the same time as, or following administration of the compound, tautomer, solvate, stereoisomer, or a pharmaceutically acceptable salt disclosed herein.

Also disclosed herein are methods of modulating, e.g., inhibiting, ferroptosis in a subject in need thereof, comprising contacting the subject with a compound of the Formulae disclosed herein, a tautomer thereof, a solvate or stereoisomer of the compound or the tautomer, or a pharmaceutically acceptable salt of the foregoing, or a pharmaceutical composition comprising any of the foregoing. In some embodiments, the methods of modulating, e.g., inhibiting, ferroptosis in a subject in need thereof comprise contacting the subject with a compound selected from Compounds 1 to 659 shown below, a tautomer thereof, a solvate or stereoisomer of the compound or the tautomer, or a pharmaceutically acceptable salt of the foregoing, or a pharmaceutical composition comprising any of the foregoing.

### DETAILED DESCRIPTION OF THE DISCLOSURE

### I. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The term “alkyl” refers to a hydrocarbon group selected from linear and branched saturated hydrocarbon groups, containing 1-20, e.g., 1-18, 1-12, 1-10, 1-8, 1-6, 1-4, or 1-3, carbon atoms. Examples of the alkyl group include methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of an alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl groups. Lower alkyl contains 1-8, preferably 1-6, more preferably 1-4 carbon atoms, and more preferably 1-3 carbon atoms.

The term “alkenyl” refers to a hydrocarbon group selected from linear and branched hydrocarbon groups, comprising at least one C═C double bond and 2-20, e.g., 2-18, 2-12, 2-10, 2-8, 2-6, or 2-4, carbon atoms. Examples of the alkenyl group may be selected from ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups. Lower alkenyl contains 2-8, preferably 2-6, and more preferably 2-4 carbon atoms.

The term “alkynyl” refers to a hydrocarbon group selected from linear and branched hydrocarbon groups, comprising at least one C≡C triple bond and 2-20, e.g., 2-18, 2-12, 2-10, 2-8, 2-6, or 2-4, carbon atoms. Examples of the alkynyl group include ethynyl, 1-propynyl, 2-propynyl (propargyl), 1-butynyl, 2-butynyl, and 3-butynyl groups. Lower alkynyl contains 2-8, preferably 2-6, and more preferably 2-4 carbon atoms.

The term “heteroalkyl” refers to an alkyl group, as defined herein, in which one or more of the constituent carbon atoms have been replaced by a heteroatom, e.g., nitrogen, oxygen, or sulfur, e.g., CH₃CH₂OH, CH₃CH₂OC₂H₅, CH₃CH₂SH, CH₃CH₂SC₂H₅, CH₃CH₂NH₂, CH₃CH₂NHC₂H₅, etc. In some embodiments, in addition to the replacement of one or more of the constituent carbon atoms by nitrogen, oxygen, or sulfur, a heteroalkyl group is further optionally substituted as defined herein.

The term “cycloalkyl” refers to a hydrocarbon group selected from saturated and partially unsaturated cyclic hydrocarbon groups, e.g., monocyclic and polycyclic (e.g., bicyclic and tricyclic) groups. For example, the cycloalkyl group may be of 3-12, or 3-8, or 3-6, or 3-4, or 5-6 carbon atoms. Even further for example, the cycloalkyl group may be a monocyclic group of 3-12, or 3-8, or 3-6, or 3-4, or 5-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e., partially unsaturated), but is not fully conjugated, and is not aromatic, as aromatic is defined herein.

The term “heterocyclic” or “heterocycle” or “heterocyclyl” refers to a ring selected from 4- to 12-membered, e.g., 3- to 6-membered, 3- to 5-membered, 4- to 5-membered, or 5- to 6-membered, monocyclic, bicyclic, and tricyclic, saturated and partially unsaturated rings comprising at least one carbon atom in addition to 1, 2, 3, or 4 heteroatoms, selected from oxygen, sulfur, and nitrogen. “Heterocycle” also refers to a 5- to 7-membered heterocyclic ring comprising at least one heteroatom selected from N, O, and S fused with 5-, 6-, and/or 7-membered cycloalkyl, carbocyclic aromatic, or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e., partially unsaturated). A heterocycle may be substituted with oxo. The point of the attachment may be carbon or heteroatom in the heterocyclic ring. A heterocycle is not a heteroaryl as defined herein.

Examples of heterocycles include, but are not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl, 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinyl, imidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl, and 1,1-dioxo-1-thiomorpholinyl.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6] and [6,6] ring systems as mentioned above; a fused bicyclic aryl ring such as 7 to 12 membered bicyclic aryl ring systems as mentioned above, a fused tricyclic aryl ring such as 10 to 15 membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The term “heteroatom” means one or more of oxygen, sulfur, nitrogen, and phosphorus, including, any oxidized form of nitrogen or sulfur; the quaternized form of any basic nitrogen or a substitutable nitrogen of a heterocyclic ring, for example N (as in 3,4-dihydro-2H-pyrrolyl), NH (as in pyrrolidinyl) or NR⁺ (wherein R is, e.g., an optionally substituted alkyl group) (as in N-substituted pyrrolidinyl).

The term “unsaturated”, as used herein, means that a moiety has one or more units or degrees of unsaturation. Unsaturation is the state in which not all of the available valence bonds in a compound are satisfied by substituents and thus the compound contains one or more double or triple bonds.

The term “alkoxy” as used herein, refers to an alkyl group, as defined above, wherein one carbon of the alkyl group is replaced by an oxygen atom, provided that the oxygen atom is linked between two carbon atoms.

The term “halogen” includes F, Cl, Br, and I, i.e., fluoro, chloro, bromo, and iodo, respectively.

As used herein, a “CN”, “cyano” or “nitrile” group refers to —C≡N.

As used herein, an “aromatic ring” refers to a carbocyclic or heterocyclic ring that contains conjugated, planar ring systems with delocalized pi electron orbitals comprised of [4n+2] p orbital electrons, wherein n is an integer of 0 to 6. A “non-aromatic” ring refers to a carbocyclic or heterocyclic that does not meet the requirements set forth above for an aromatic ring, and can be either completely or partially saturated. Non-limiting examples of aromatic rings include aryl and heteroaryl rings that are further defined as follows. An “aromatic ring” may be depicted as a cycle with conjugated double bonds, such as

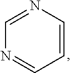

or as a cycle with an inside circle such as

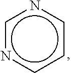

or as otherwise indicated, such as

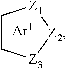

wherein Ar¹ is indicated as aromatic or non-aromatic.

The term “aryl” herein refers to a group selected from: monocyclic carbocyclic aromatic rings, for example, phenyl; bicyclic ring systems such as 7-12 membered bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group may be a 6-membered carbocyclic aromatic ring fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene.

The term “heteroaryl” refers to a group selected from: 5- to 7-membered, e.g., 5- to 6-membered, aromatic, monocyclic rings comprising 1, 2, 3, or 4 heteroatoms selected from N, O, and S, with the remaining ring atoms being carbon; 8- to 12-membered bicyclic rings comprising 1, 2, 3, or 4 heteroatoms, selected from N, O, and S, with the remaining ring atoms being carbon and wherein at least one ring is aromatic and at least one heteroatom is present in the aromatic ring; and 11- to 14-membered tricyclic rings comprising 1, 2, 3, or 4 heteroatoms, selected from N, O, and S, with the remaining ring atoms being carbon and wherein at least one ring is aromatic and at least one heteroatom is present in an aromatic ring.

For example, the heteroaryl group may be a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinolinyl.

The term “acyl” refers to a substituent group where a point of attachment in the substituent group is a carbonyl. Exemplary acyl groups include, but are not limited to, —C(═O)R′, —C(═O)NR′R″, or —C(═O)OR′, wherein R′ and R″ are independently selected from hydrogen, alkyl, alkenyl, alkynyl, heteroalkyl, heteroalkenyl, heteroalkynyl, cycloalkyl, aryl, heterocyclyl, or heteroaryl, any of which may be further substituted by one or more substituents.

Some of the compounds may exist with different points of attachment of hydrogen, referred to as “tautomers.” For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

The compounds, tautomers, solvates, or pharmaceutically acceptable salts of the disclosure may contain an asymmetric center and may thus exist as enantiomers. For example, where the compounds possess two or more asymmetric centers, they may additionally exist as diastereoisomers. Enantiomers and diastereoisomers fall within the broader class of stereoisomers. All such possible stereoisomers as substantially pure resolved enantiomers, racemic mixtures thereof, as well as mixtures of diastereoisomers are intended to be included in this disclosure. All stereoisomers of the compounds, tautomers, solvates, and pharmaceutically acceptable salts thereof are intended to be included. Unless specifically mentioned otherwise, reference to one isomer applies to any of the possible isomers. Whenever the isomeric composition is unspecified, all possible isomers are included.

Diastereoisomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereoisomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereoisomers using optically active resolving agents. Racemic mixtures of chiral compounds of the disclosure can be separated and isolated by any suitable method, including: (1) formation of ionic, diastereoisomeric salts with chiral compounds and separation by fractional crystallization or other methods, (2) formation of diastereoisomeric compounds with chiral derivatizing reagents, separation of the diastereoisomers, and conversion to the pure stereoisomers, and (3) separation of the substantially pure or enriched stereoisomers directly under chiral conditions.

The term “substantially pure” in the context of stereoisomers means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

Unless otherwise indicated, structures depicted herein are meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the compounds disclosed herein are within the scope of the disclosure. Unless otherwise stated, all tautomeric forms of the compounds of the disclosure are within the scope of the disclosure.

The disclosure provides pharmaceutically acceptable salts of the disclosed compounds, tautomers, solvates, and stereoisomers. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure.

“Pharmaceutically acceptable salts” include, but are not limited to salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)ₙ—COOH, wherein n is selected from 0 to 4. Similarly, examples of pharmaceutically acceptable cations include, but are not limited to, sodium, potassium, calcium, magnesium, aluminum, lithium, and ammonium. Suitable pharmaceutically acceptable salts are, for example, those disclosed in S. M. Berge, et al. J. Pharmaceutical Sciences, 1977, 66, pp. 1 to 19.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, benzenesulfonic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate (i.e., caprate), caprylate, acrylate, formate, isobutyrate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, P-hydroxybutyrate, glycolate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium salts. Further non-limiting examples of pharmaceutically acceptable salts include salts of ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

If a compound is obtained as an acid addition salt, the free base can be obtained by basifying a solution of the acid addition salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The compounds, tautomers, solvates, stereoisomers, and pharmaceutically acceptable salts of the disclosure may also contain unnatural proportions of atomic isotopes at one or more of the atoms that constitute such compounds. For example, —CD₃, —CD₂H or —CDH₂ contains one or more deuteriums in place of hydrogen. For example, the compounds may be radiolabeled with radioactive isotopes, such as for example tritium (³H), iodine-125 (¹²⁵I) or carbon-14 (¹⁴C). All isotopic variations of the compounds of the disclosure, whether radioactive or not, are intended to be encompassed within the scope of the disclosure.

As used herein, “optionally substituted” is interchangeable with the phrase “substituted or unsubstituted.” In general, the term “substituted,” refers to the replacement of a hydrogen radical in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by this disclosure are those that result in the formation of stable or chemically feasible compounds.

In some embodiments, substituents are independently selected from optionally substituted heteroatom and optionally substituted, optionally hetero-, optionally cyclic C₁-C₁₈ hydrocarbyl, particularly wherein the optionally substituted, optionally hetero-, optionally cyclic C₁-C₁₈ hydrocarbyl is optionally-substituted, optionally hetero-, optionally cyclic alkyl, alkenyl or alkynyl, or optionally-substituted, optionally hetero-, aryl; and/or the optionally substituted heteroatom is halogen, optionally substituted hydroxyl (such as alkoxy, aryloxy), optionally substituted acyl (such as formyl, alkanoyl, carbamoyl, carboxyl, amido), optionally substituted amino (such as amino, alkylamino, dialkylamino, amido, sulfamidyl), optionally substituted thiol (such as mercapto, alkylthiol, aryl thiol), optionally substituted sulfinyl or sulfonyl (such as alkylsulfinyl, arylsulfinyl, alkyl sulfonyl, arylsulfonyl), nitro, or cyano.

In some embodiments, substituents are independently selected from: halogen, —R′, —OR′, ═O, ═NR′, ═N—OR′, —NR′R″, —SR′, —SiR′R″R′, —OC(═O)R′, —C(═O)R′, —CO₂R′, —C(═O)NR′R″, —OC(═O)NR′R″, —NR″C(═O)R′, —NR′—C(═O)NR″R′″, —NR′—SO₂NR″R″′, —NR″CO₂R′, —NH—C(NH₂)═NH, —NR′C(NH₂)═NH, —NH—C(NH₂)═NR′, —S(O)R′, —SO₂R′, —SO₂NR′R″, —NR″SO₂R, —CN, —NO₂, —N₃, —CH(Ph)₂, perfluoro(C₁-C₄)alkoxy, and perfluoro(C₁-C₄)alkyl, in a number ranging from zero to three, with those groups having zero, one, or two substituents being particularly preferred. R′, R″ and R″′ each independently refer to hydrogen, unsubstituted C₁-C₈ alkyl and heteroalkyl, C₁-C₈ alkyl and heteroalkyl substituted with one to three halogens, unsubstituted aryl, aryl substituted with one to three halogens, unsubstituted alkyl, alkoxy, or thioalkoxy groups, or aryl-(C₁-C₄) alkyl groups. When R′ and R″ are attached to the same nitrogen atom, they can be combined with the nitrogen atom to form a 5-, 6- or 7-membered ring. Hence, —NR′R″ includes, e.g., 1-pyrrolidinyl and 4-morpholinyl. When the aryl group is 1,2,3,4-tetrahydronaphthalenyl, it may be substituted with a substituted or unsubstituted C₃-C₇ spirocycloalkyl group. The C₃-C₇ spirocycloalkyl group may be substituted in the same manner as defined herein for “cycloalkyl.”

In some embodiments, substituents are selected from: halogen, —R′, —OR′, ═O, —NR′R″, —SR′, —SiR′R″R″′, —OC(═O)R′, —C(═O)R′, —CO₂R′, —C(═O)NR′R″, —OC(═O)NR′R″, —NR″C(═O)R′, —NR″CO₂R′, —NR′—SO₂NR″R″′, —S(═O)R′, —SO₂R′, —SO₂NR′R″, —NR″SO₂R, —CN, —NO₂, perfluoro C₁-C₄ alkoxy and perfluoro C₁-C₄ alkyl, where R′ and R″ are as defined above.

In some embodiments, substituents are independently selected from substituted or unsubstituted heteroatom, substituted or unsubstituted, 0-3 heteroatom-containing C₁-C₆ alkyl (e.g., C₁-C₃ alkyl or C₁-C₂ alkyl), substituted or unsubstituted, 0-3 heteroatom-containing C₂-C₆ alkenyl (e.g., C₂-C₄ alkenyl), substituted or unsubstituted, 0-3 heteroatom-containing C₂-C₆ alkynyl (e.g., C₂-C₄ alkynyl), or substituted or unsubstituted, 0-3 heteroatom-containing C₆-C₁₄ aryl (e.g., C₅-C₆ aryl), wherein each heteroatom is independently oxygen, phosphorus, sulfur, or nitrogen.

In some embodiments, substituents are independently selected from aldehyde, aldimine, alkanoyloxy, alkoxy, alkoxycarbonyl, alkyloxy, alkyl, alkenyl, alkynyl, amine, azo, halogen, carbamoyl, carbonyl, carboxamido, carboxyl, cyanyl, ester, haloformyl, hydroperoxyl, hydroxyl, imine, isocyanide, iscyante, N-tert-butoxycarbonyl, nitrate, nitrile, nitrite, nitro, nitroso, phosphate, phosphono, sulfide, sulfonyl, sulfo, sulfhydryl, thiol, thiocyanyl, trifluoromethyl, and trifluromethyl ether (OCF₃) groups.

Preferred substituents are disclosed herein and exemplified in the tables, structures, examples, and claims, and may be applied across different compounds of this disclosure. For example, substituents of a given compound may be combinatorically used with other compounds.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example, reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art may apply such techniques to achieve a desired separation.

Non-limiting examples of suitable solvents that may be used in this disclosure include water, methanol (MeOH), ethanol (EtOH), dichloromethane or methylene chloride (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH), and sodium methoxide (NaOMe; NaOCH₃).

The term “subject” refers to an animal including a human.

The term “therapeutically effective amount” refers to the amount of a compound that produces a desired effect for which it is administered (e.g., improvement in a disease or condition, lessening the severity of a disease or condition, and/or reducing progression of a disease or condition, e.g., a disease or condition selected from neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. The disease or condition may be involved with dysregulated, e.g., abnormal, ferroptosis. The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, curing a disease or condition or a symptom thereof, lower risk of a disease or condition, e.g., neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. The disease or condition may be involved with dysregulated, e.g., abnormal, ferroptosis. Improvements in or lessening the severity of any of these symptoms can be assessed according to methods and techniques known in the art.

The terms “about” and “approximately,” when used in connection with a number such as a percentage include the number as specified, and a range of the number (e.g., a range of percentages, for example, a range of ±10% with respect to a specific point value) that is recognized by one of ordinary skill in the art.

### II. Compounds and Compositions

In a first embodiment, a compound of this disclosure is a compound of the following structural Formula I:

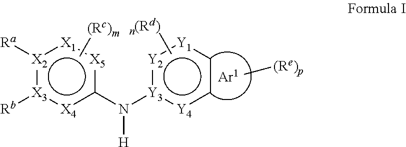

a tautomer thereof, a solvate or stereoisomer of the compound or the tautomer, or a pharmaceutically acceptable salt of the foregoing, wherein:
Ar¹ is aryl, cycloalkyl, heteroaryl, or 5- to 7-membered heterocyclyl;
X₁ is C, N, or S;
X₂, X₃, X₅, Y₁, Y₂, Y₃, and Y₄ are each independently C or N;
X₄ is C or N, but when X₁ is S, X₄ is nothing;
R^{a} is selected from H, halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl;
R^{b} is selected from H, halogen, CN, optionally substituted heteroatom, optionally substituted alkyl, optionally substituted cycloalkyl, and optionally substituted heterocyclyl;
R^{a} and R^{b} may join together to form an optionally substituted 3- to 10-membered carbocyclic, heterocyclic, aromatic, or heteroaromatic ring;
R^{c}, for each occurrence, is independently selected from halogen, CN, optionally substituted heteroatom, and optionally substituted alkyl;
R^{d}, for each occurrence, is independently selected from halogen, CN, optionally substituted heteroatom, and optionally substituted alkyl;
R^{e}, for each occurrence, is independently selected from halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl; and
m, n, and p are each an integer independently selected from 0, 1, 2, and 3;
provided that:
(1)

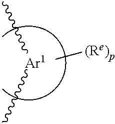

is

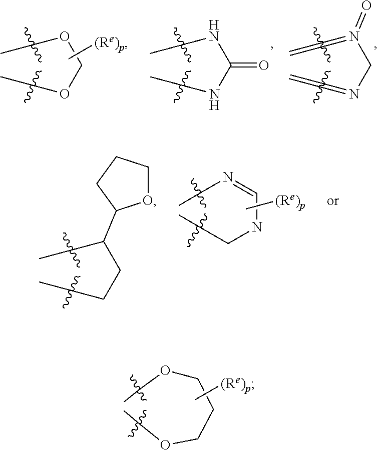

(2) when

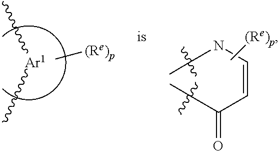

R^{e} is not —C(═O)OH or —C(═O)O(C₁-C₃-alkyl);
(3) when

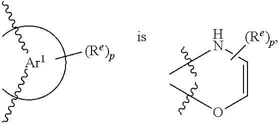

R^{a} is not OH; and
(4) when Ar¹ is a 5- to 6-membered cycloalkyl, 5- to 6-membered heteroaryl, or 5- to 7-membered heterocyclyl, (i) R^{a} is selected from optionally substituted cycloalkyl, optionally substituted heterocyclyl, optionally substituted aryl, and optionally substituted heteroaryl, (ii) R^{a} is selected from H, halogen, CN, optionally substituted heteroatom, optionally substituted alkyl, optionally substituted alkenyl, optionally substituted alkynyl, provided that p is an integer selected from 1, 2, and 3 and that at least one of the R^{e} is ═O, or (iii) R^{a} and R^{b} join together to form an optionally substituted 3- to 10-membered carbocyclic, heterocyclic, aromatic, or heteroaromatic ring, provided that p is an integer selected from 1, 2, and 3 and that at least one of the R^{e} is ═O.

Combinations of substituents as disclosed herein are those that result in the formation of stable or chemically feasible compounds. For abbreviation or according to common practice, certain hydrogen atoms attached to a certain atom (e.g., a carbon atom C or a nitrogen atom N) are not specifically spelled out in a chemical structure, formula, or notation; hydrogen atoms are deemed to be present to the extent the valences of the certain atom (e.g., C or N) are completed.

In a second embodiment, in a compound, tautomer, a solvate or stereoisomer of the compound or the tautomer, or pharmaceutically acceptable salt of this disclosure, Ar¹ is 5- to 6-membered heteroaryl or 5- to 7-membered heterocyclyl; and all other variables not specifically defined herein are as defined in the preceding embodiment.

In a third embodiment, a compound of the disclosure is a compound of the following structural Formula IIa:

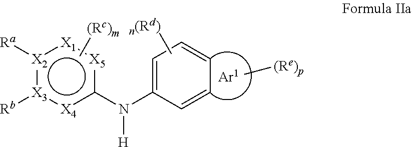

a tautomer thereof, a solvate or stereoisomer of the compound or the tautomer, or a pharmaceutically acceptable salt of the foregoing; and all other variables not specifically defined herein are as defined in any one of the preceding embodiments.

In a fourth embodiment, a compound of the disclosure is a compound of the following structural Formula IIb:

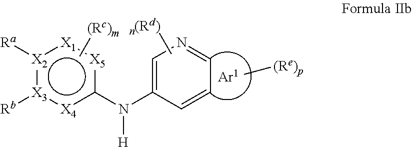

a tautomer thereof, a solvate or stereoisomer of the compound or the tautomer, or a pharmaceutically acceptable salt of the foregoing; and all other variables not specifically defined herein are as defined in any one of the preceding embodiments.

In a fifth embodiment, a compound of the disclosure is a compound of the following structural Formula IIIa:

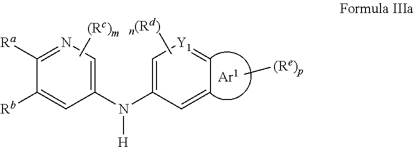

a tautomer thereof, a solvate or stereoisomer of the compound or the tautomer, or a pharmaceutically acceptable salt of the foregoing; and all other variables not specifically defined herein are as defined in any one of the preceding embodiments.

In a sixth embodiment, a compound of the disclosure is a compound of the following structural Formula IIIb:

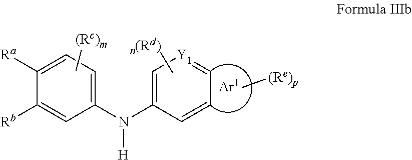

a tautomer thereof, a solvate or stereoisomer of the compound or the tautomer, or a pharmaceutically acceptable salt of the foregoing; and all other variables not specifically defined herein are as defined in any one of the preceding embodiments.

In a seventh embodiment, a compound of the disclosure is a compound of the following structural Formula IIIc:

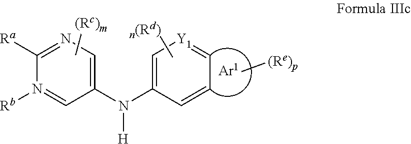

a tautomer thereof, a solvate or stereoisomer of the compound or the tautomer, or a pharmaceutically acceptable salt of the foregoing; and all other variables not specifically defined herein are as defined in any one of the preceding embodiments.

In an eighth embodiment, a compound of the disclosure is a compound of the following structural Formula IIId:

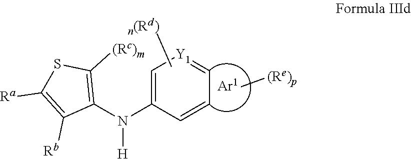

a tautomer thereof, a solvate or stereoisomer of the compound or the tautomer, or a pharmaceutically acceptable salt of the foregoing; and all other variables not specifically defined herein are as defined in any one of the preceding embodiments.

In a ninth embodiment, a compound of the disclosure is a compound of the following structural Formula IVa:

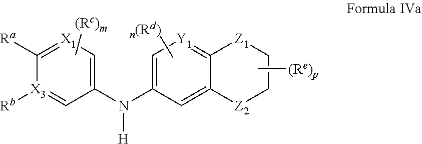

a tautomer thereof, a solvate or stereoisomer of the compound or the tautomer, or a pharmaceutically acceptable salt of the foregoing; wherein X₁ and X₃ are each independently C or N; and Z₁ and Z₂ are each independently N or O; and all other variables not specifically defined herein are as defined in any one of the preceding embodiments.

In a tenth embodiment, a compound of the disclosure is a compound of the following structural Formula IVb:

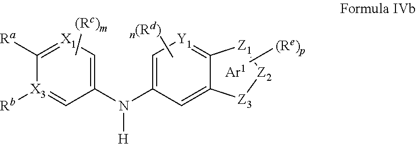

a tautomer thereof, a solvate or stereoisomer of the compound or the tautomer, or a pharmaceutically acceptable salt of the foregoing; wherein X₁ and X₃ are each independently C or N; Ar¹ is aromatic or non-aromatic; and Z₁, Z₂, and Z₃ are each independently N or C; and all other variables not specifically defined herein are as defined in any one of the preceding embodiments.

In an eleventh embodiment, a compound of the disclosure is a compound of the following structural Formula IVc:

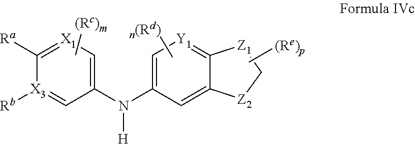

a tautomer thereof, a solvate or stereoisomer of the compound or the tautomer, or a pharmaceutically acceptable salt of the foregoing; wherein X₁ and X₃ are each independently C or N; and Z₁ and Z₂ are each independently O or N; and all other variables not specifically defined herein are as defined in any one of the preceding embodiments.

In a twelfth embodiment, a compound of the disclosure is a compound of the following structural Formula IVd:

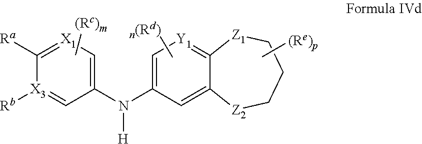

a tautomer thereof, a solvate or stereoisomer of the compound or the tautomer, or a pharmaceutically acceptable salt of the foregoing; wherein X₁ and X₃ are each independently C or N; and Z₁ and Z₂ are each independently O or N; and all other variables not specifically defined herein are as defined in any one of the preceding embodiments.

In a thirteenth embodiment, a compound of the disclosure is a compound of the following structural Formula Va:

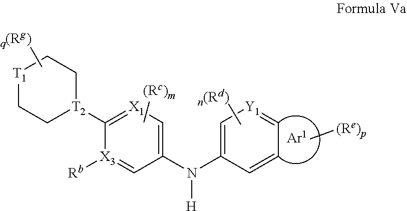

a tautomer thereof, a solvate or stereoisomer of the compound or the tautomer, or a pharmaceutically acceptable salt of the foregoing; wherein X₁ and X₃ are each independently N or C; T₁ is C or O; T₂ is C or N; R^{g}, for each occurrence, is selected from CF₃, —CH₃, —OCH₃, and —CH₂CH₃; q is 0, 1, or 2; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; wherein the C₁ to C₃ alkoxy and C₁ to C₃ alkyl of R^{b} and R^{c}, for each occurrence, are each optionally substituted with 1 to 3 groups of halogen; and all other variables not specifically defined herein are as defined in any one of the preceding embodiments.

In a fourteenth embodiment, a compound of the disclosure is a compound of the following structural Formula Vb:

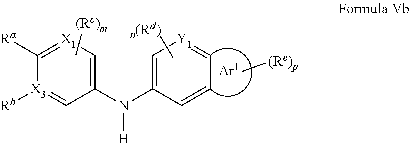

a tautomer thereof, a solvate or stereoisomer of the compound or the tautomer, or a pharmaceutically acceptable salt of the foregoing; wherein X₁ and X₃ are each independently N or C; R^{a} is selected from C₁ to C₃ alkoxy and C₁ to C₃ alkyl; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; wherein the C₁ to C₃ alkoxy and C₁ to C₃ alkyl of R^{a}, R^{b}, and R^{c}, for each occurrence, are each optionally substituted with 1 to 3 groups of halogen; and all other variables not specifically defined herein are as defined in any one of the preceding embodiments.

In a fifteenth embodiment, a compound of the disclosure is a compound of the following structural Formula VIa:

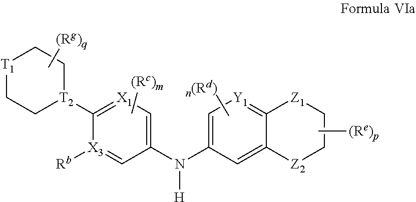

a tautomer thereof, a solvate or stereoisomer of the compound or the tautomer, or a pharmaceutically acceptable salt of the foregoing; wherein X₁ and X₃ are each independently N or C; T₁ is C or O; T₂ is C or N; R^{g}, for each occurrence, is independently selected from CF₃, —CH₃, —OCH₃, and —CH₂CH₃; q is 0, 1, or 2; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; the C₁ to C₃ alkoxy and C₁ to C₃ alkyl of R^{b} and R^{c}, for each occurrence, are each optionally substituted with 1 to 3 groups of halogen; Z₁ and Z₂ are each independently N or O; and all other variables not specifically defined herein are as defined in any one of the preceding embodiments.

In a sixteenth embodiment, a compound of the disclosure is a compound of the following structural Formula VIb:

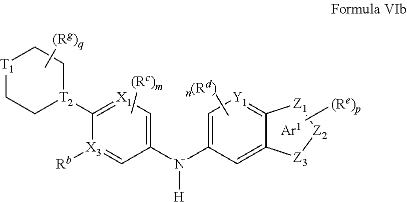

a tautomer thereof, a solvate or stereoisomer of the compound or the tautomer, or a pharmaceutically acceptable salt of the foregoing; wherein X₁ and X₃ are each independently N or C; T₁ is C or O; T₂ is C or N; R^{g}, for each occurrence, is independently selected from CF₃, —CH₃, —OCH₃, and —CH₂CH₃; q is 0, 1, or 2; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; the C₁ to C₃ alkoxy and C₁ to C₃ alkyl of R^{b} and R^{c}, for each occurrence, are each optionally substituted with 1 to 3 groups of halogen; Ar¹ is aromatic or non-aromatic; and Z₁, Z₂, and Z₃ are each independently N or C; and all other variables not specifically defined herein are as defined in any one of the preceding embodiments.

In a seventeenth embodiment, a compound of the disclosure is a compound of the following structural Formula VIc:

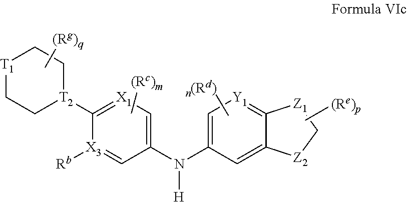

a tautomer thereof, a solvate or stereoisomer of the compound or the tautomer, or a pharmaceutically acceptable salt of the foregoing; wherein X₁ and X₃ are each independently N or C; T₁ is C or O; T₂ is C or N; R^{g}, for each occurrence, is independently selected from CF₃, —CH₃, —OCH₃, and —CH₂CH₃; q is 0, 1, or 2; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; the C₁ to C₃ alkoxy and C₁ to C₃ alkyl of R^{b} and R^{c}, for each occurrence, are each optionally substituted with 1 to 3 groups of halogen; and Z₁ and Z₂ are each independently O or N; and all other variables not specifically defined herein are as defined in any one of the preceding embodiments.

In an eighteenth embodiment, a compound of the disclosure is a compound of the following structural Formula VId:

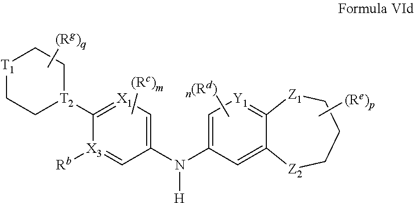

a tautomer thereof, a solvate or stereoisomer of the compound or the tautomer, or a pharmaceutically acceptable salt of the foregoing; wherein X₁ and X₃ are each independently N or C; T₁ is C or O; T₂ is C or N; R^{g}, for each occurrence, is independently selected from CF₃, —CH₃, —OCH₃, and —CH₂CH₃; q is 0, 1, or 2; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; the C₁ to C₃ alkoxy and C₁ to C₃ alkyl of R^{b} and R^{c}, for each occurrence, are each optionally substituted with 1 to 3 groups of halogen; and Z₁ and Z₂ are each independently O or N; and all other variables not specifically defined herein are as defined in any one of the preceding embodiments.

In a nineteenth embodiment, a compound of the disclosure is a compound of the following structural Formula VIIa:

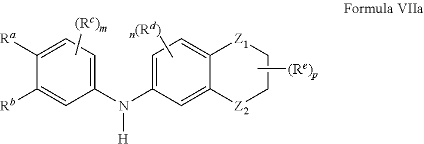

a tautomer thereof, a solvate or stereoisomer of the compound or the tautomer, or a pharmaceutically acceptable salt of the foregoing; wherein Z₁ and Z₂ are each independently N or O; R^{a} is selected from C₁ to C₃ alkoxy and C₁ to C₃ alkyl; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; wherein the C₁ to C₃ alkoxy and C₁ to C₃ alkyl of R^{a}, R^{b}, and R^{c}, for each occurrence, are each optionally substituted with 1 to 3 groups of halogen; and all other variables not specifically defined herein are as defined in any one of the preceding embodiments.

In a twentieth embodiment, a compound of the disclosure is a compound of the following structural Formula VIIb:

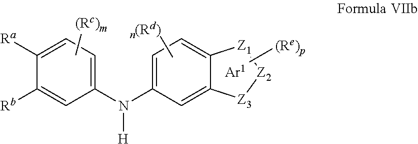

a tautomer thereof, a solvate or stereoisomer of the compound or the tautomer, or a pharmaceutically acceptable salt of the foregoing; wherein Ar¹ is aromatic or non-aromatic; Z₁, Z₂, and Z₃ are each independently N or C; R^{a} is selected from C₁ to C₃ alkoxy and C₁ to C₃ alkyl; R^{b} is selected from H, halogen, C₁ to C₃ alkoxy, and C₁ to C₃ alkyl; and R^{c} is selected from halogen, C₁ to C₃ alkoxy, and C, to C₃ alkyl; wherein the C₁ to C₃ alkoxy and C, to C₃ alkyl of R^{a}, R^{b}, and R^{c}, for each occurrence, are each optionally substituted with 1 to 3 groups of halogen; and all other variables not specifically defined herein are as defined in any one of the preceding embodiments.

In a twenty-first embodiment, a compound of the disclosure is a compound of the following structural Formula VIIc:

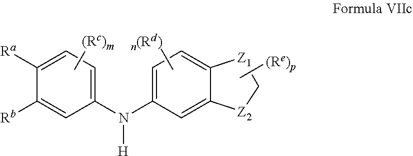

a tautomer thereof, a solvate or stereoisomer of the compound or the tautomer, or a pharmaceutically acceptable salt of the foregoing, wherein Z₁ and Z₂ are each independently O or N; R^{a} is selected from C₁ to C₃ alkoxy and C₁ to C₃ alkyl; R^{b} is selected from H, halogen, C₁ to C₃ alkoxy, and C, to C₃ alkyl; and R^{c} is selected from halogen, C₁ to C₃ alkoxy, and C₁ to C₃ alkyl; wherein the C₁ to C₃ alkoxy and C, to C₃ alkyl of R^{a}, R^{b}, and R^{c}, for each occurrence, are each optionally substituted with 1 to 3 groups of halogen; and all other variables not specifically defined herein are as defined in any one of the preceding embodiments.

In a twenty-second embodiment, a compound of the disclosure is a compound of the following structural Formula VIId:

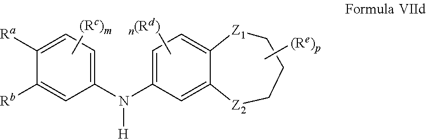

a tautomer thereof, a solvate or stereoisomer of the compound or the tautomer, or a pharmaceutically acceptable salt of the foregoing, wherein Z₁ and Z₂ are each independently O or N; R^{a} is selected from C₁ to C₃ alkoxy and C₁ to C₃ alkyl; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; wherein the C₁ to C₃ alkoxy and C₁ to C₃ alkyl of R^{a}, R^{b}, and R^{c}, for each occurrence, are each optionally substituted with 1 to 3 groups of halogen; and all other variables not specifically defined herein are as defined in any one of the preceding embodiments.

In a twenty-third embodiment, a compound of the disclosure is a compound of the following structural Formulae VIIIa-VIIII:

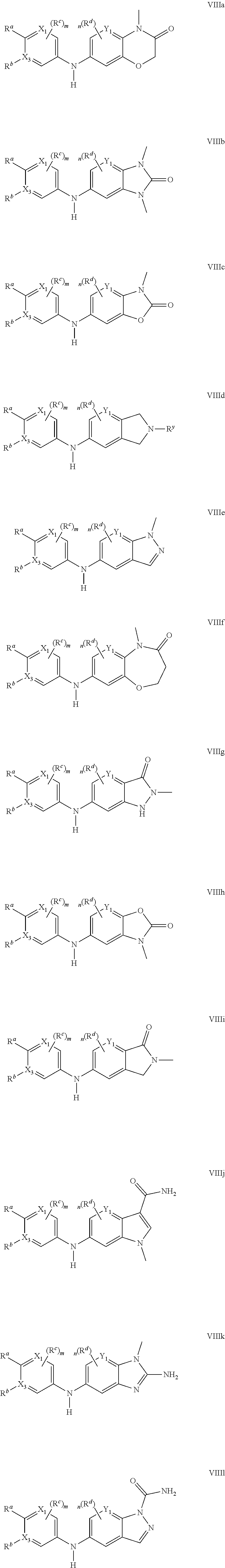

a tautomer thereof, a solvate or stereoisomer of the compound or the tautomer, or a pharmaceutically acceptable salt of the foregoing, wherein: X₁ and X₃ are each independently C or N;
R^{a} is selected from

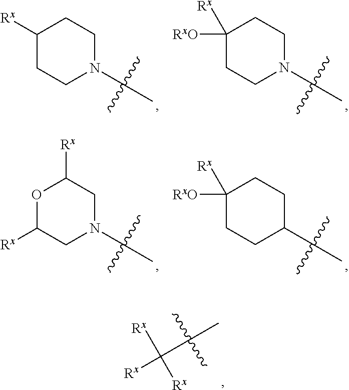

—OR^{x}, and —CH₂R^{x}, wherein R^{x}, for each occurrence, is independently selected from CF₃, —CH₃, and —CH₂CH₃;
R^{b} is selected from H, halogen, C₁ to C₂ alkyl, and C₁ to C₂ alkoxy;
R^{c} is selected from halogen, C₁ to C₂ alkyl, and C₁ to C₂ alkoxy;
R^{d} is selected from halogen, C₁ to C₂ alkyl, and C₁ to C₂ alkoxy;
R^{y} is selected from H and C₁ to C₂ alkyl;
m is 0, 1, or 2; and
n is 0 or 1;
and all other variables not specifically defined herein are as defined in any one of the preceding embodiments.

In a twenty-fourth embodiment, in a compound, tautomer, a solvate or stereoisomer of the compound or the tautomer, or pharmaceutically acceptable salt of this disclosure, R^{a} is selected from hydrogen, halogen, cyano, C₁-C₁₀ alkyl, C₂-C₁₀ alkenyl, C₂-C₁₀ alkynyl, C₃-C₁₀ cycloalkyl, 3- to 10-membered heterocyclyl, phenyl, 5- to 7-membered heteroaryl, —C(═O)R^{s}, —C(═O)OR^{s}, —NR^{p}R^{q}, —OR^{s}, wherein the C₃-C₁₀ cycloalkyl, 3- to 10-membered heterocyclyl, phenyl, and 5- to 7-membered heteroaryl of R^{a} are each optionally substituted with 1-3 groups selected from halogen, OR^{s}, and C₁-C₁₀ alkyl optionally substituted with 1 to 3 groups of halogen;
the C₁-C₁₀ alkyl, C₂-C₁₀ alkenyl, and C₂-C₁₀ alkynyl of R^{a} are each optionally substituted with 1 to 3 groups selected from halogen, 3- to 10-membered heterocyclyl, and —OR^{s};
R^{p} and R^{q}, for each occurrence, are independently selected from hydrogen, C₃-C₁₀ cycloalkyl, and C₁-C₁₀ alkyl, wherein the C₃-C₁₀ cycloalkyl and C₁-C₁₀ alkyl of R^{p} and R^{q} are each optionally substituted with 1 to 3 groups selected from halogen, —OR^{s} and —C(═O)OR^{s};
R^{s}, for each occurrence, is selected from H, C₃-C₁₀ cycloalkyl, 3- to 10-membered heterocyclyl, 5- to 6-membered aryl, and C₁-C₁₀ alkyl optionally substituted with 1 to 3 groups selected from halogen, C₃-C₁₀ cycloalkyl, 3- to 10-membered heterocyclyl, and C₁-C₆ alkoxy, wherein the C₃-C₁₀ cycloalkyl and 3- to 10-membered heterocyclyl of R^{s} and the C₃-C₁₀ cycloalkyl and 3- to 10-membered heterocyclyl of the C₁-C₁₀ alkyl of R^{s} are each optionally substituted with 1 to 3 groups of C₁-C₆ alkyl optionally substituted with halogen, and the C₁-C₆ alkoxy of the C₁-C₁₀ alkyl of R^{s} is optionally substituted with 1 to 3 groups of halogen; and
all other variables not specifically defined herein are as defined in any one of the preceding embodiments.

In a twenty-fifth embodiment, in a compound, tautomer, a solvate or stereoisomer of the compound or the tautomer, or pharmaceutically acceptable salt of this disclosure, R^{a} is selected from H, F methyl,

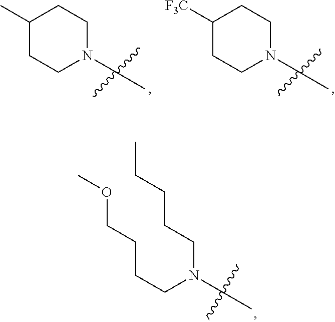

—O(CH₂)₃CH₃, —(CH₂)₄CH₃,

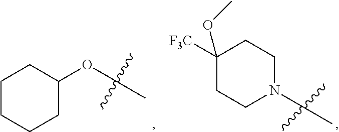 [SMILES: *OC1CCCCC1 ; *N1CCC(OC)(C(F)(F)F)CC1]

—O(CH₂)₂CH₂F,

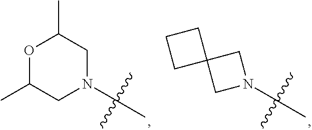

—N(CH₂CH₃)₂,

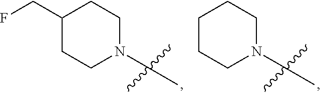 [SMILES: *N1CCC(CF)CC1 ; *N1CCCCC1]

—N(CH₂CH₃)(CH₂)₄CH₃, —N(CH₂CH₃)(CH₂)₃OCH₃, —O(CH₂)₄CH₃,

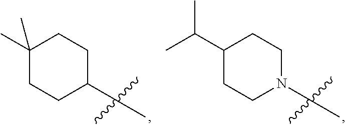

—C(CH₃)₂CH₂CH₃, —C(CH₃)₃,

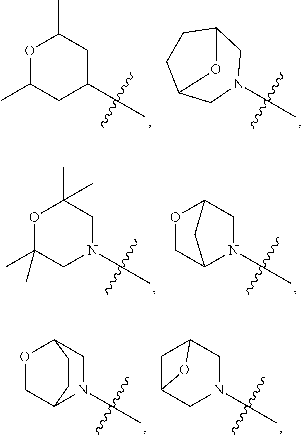

—N(CH₃)CH₂CF₃,

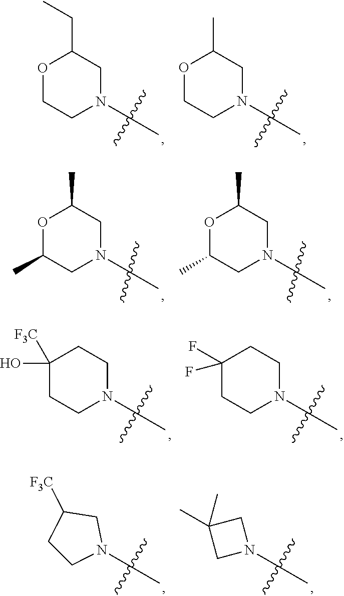

—N(CH₃)₂,

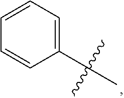 [SMILES: *c1ccccc1]

—O(CH₂)₂OCH₃,

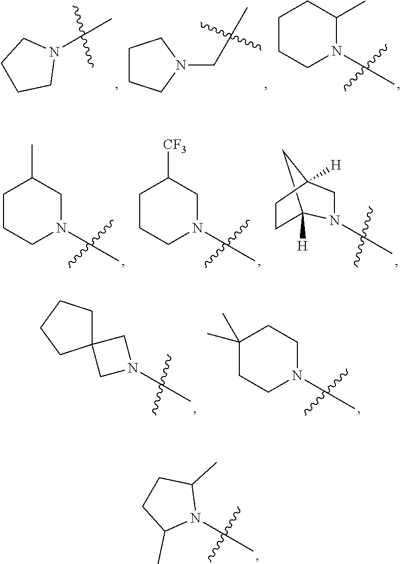

—C(═O)CF₃, —N(CH₂CH₃)(CH₂)₄CH₃, —N(CH₂CH₃)(CH₂)₃OCH₃,

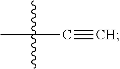 [SMILES: *C#C]

—O(CH₂)₄CH₃, —OCH₂CH₃,

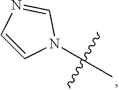 [SMILES: *n1ccnc1]

—C(═O)OCH₂CH₃, —OCH₃,

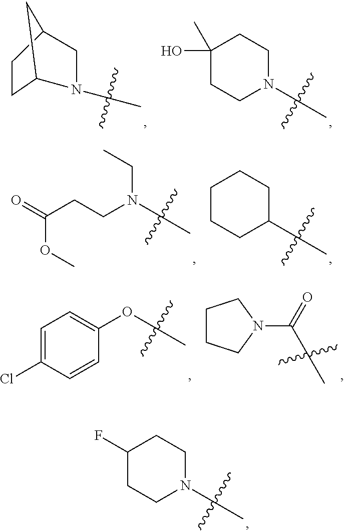

—N(CH₃)(CH₂CH₂OCH₂OCH₂CH₃); all other variables not specifically defined herein are as defined in any one of the preceding embodiments.

In a twenty-sixth embodiment, in a compound, tautomer, a solvate or stereoisomer of the compound or the tautomer, or pharmaceutically acceptable salt of this disclosure, R^{a} is selected from

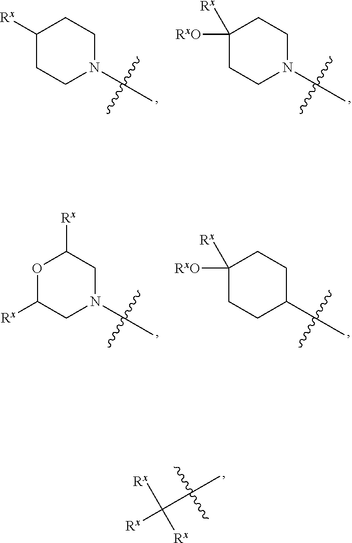

—OR^{x}, and —CH₂R^{x}, wherein R^{x}, for each occurrence, is independently selected from C₁ to C₂ alkyl optionally substituted with 1 to 3 groups of halogen; all other variables not specifically defined herein are as defined in any one of the preceding embodiments.

In a twenty-seventh embodiment, in a compound, tautomer, a solvate or stereoisomer of the compound or the tautomer, or pharmaceutically acceptable salt of this disclosure, R^{a} is selected from

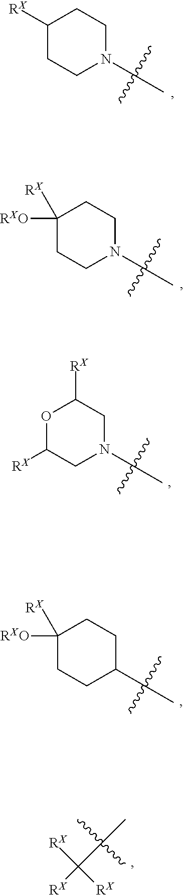

—OR^{x}, and —CH₂R^{x}, wherein R^{x}, for each occurrence, is independently selected from CF₃, —CH₃, and —CH₂CH₃; all other variables not specifically defined herein are as defined in any one of the preceding embodiments.

In a twenty-eighth embodiment, in a compound, tautomer, a solvate or stereoisomer of the compound or the tautomer, or pharmaceutically acceptable salt of this disclosure, R^{b} is selected from is H, halogen, C₁ to C₆ alkyl, —NR^{p}R^{q}, CN, C₁-C₆ alkoxy, and 5- to 6-membered heterocyclyl optionally substituted with C₁-C₃ alkyl optionally substituted with 1 to 3 groups selected from halogen, wherein R^{p} and R^{q} are each selected from C₁ to C₆ alkyl, and wherein the C₁-C₆ alkyl and C₁-C₆ alkoxy of R^{b} and the C₁-C₆ alkyl of R^{p} and R^{q} are each optionally substituted with 1 to 3 groups selected from halogen; all other variables not specifically defined herein are as defined in any one of the preceding embodiments.

In a twenty-ninth embodiment, in a compound, tautomer, a solvate or stereoisomer of the compound or the tautomer, or pharmaceutically acceptable salt of this disclosure, R^{b} is H, methyl, —NHCH₃, —N(CH₃)₂, —O(CH₂)₂CH₃, F, —OCH₃, —CF₃, Cl, —N(CH₃)₂, and

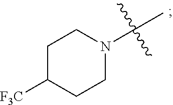 [SMILES: *N1CCC(C(F)(F)F)CC1]

all other variables not specifically defined herein are as defined in any one of the preceding embodiments.

In a thirtieth embodiment, in a compound, tautomer, a solvate or stereoisomer of the compound or the tautomer, or pharmaceutically acceptable salt of this disclosure, R^{b} is selected from halogen, C₁-C₂ alkyl, and C₁-C₂ alkoxy; all other variables not specifically defined herein are as defined in any one of the preceding embodiments.

In a thirty-first embodiment, in a compound, tautomer, a solvate or stereoisomer of the compound or the tautomer, or pharmaceutically acceptable salt of this disclosure, R^{b} is selected from F, methyl, and —OCH₃; all other variables not specifically defined herein are as defined in any one of the preceding embodiments.

In a thirty-second embodiment, in a compound, tautomer, a solvate or stereoisomer of the compound or the tautomer, or pharmaceutically acceptable salt of this disclosure, R^{c}, for each occurrence, is independently selected from is halogen, C₁-C₆ alkyl, —NR^{p}R^{q}, CN, and C₁-C₆ alkoxy, wherein R^{p} and R^{q} are each selected from C₁ to C₆ alkyl, and wherein the C₁-C₆ alkyl and C₁-C₆ alkoxy of R^{c} and the C₁-C₆ alkyl of R^{p} and R^{q} are each optionally substituted with 1 to 3 groups selected from halogen; all other variables not specifically defined herein are as defined in any one of the preceding embodiments.

In a thirty-third embodiment, in a compound, tautomer, a solvate or stereoisomer of the compound or the tautomer, or pharmaceutically acceptable salt of this disclosure, R^{c} is selected from methyl, F, —OCH₃, Cl, —N(CH₃)₂, and CN; all other variables not specifically defined herein are as defined in any one of the preceding embodiments.

In a thirty-fourth embodiment, in a compound, tautomer, a solvate or stereoisomer of the compound or the tautomer, or pharmaceutically acceptable salt of this disclosure, R^{c} is selected from halogen, C₁-C₂ alkyl, and C₁-C₂ alkoxy; all other variables not specifically defined herein are as defined in any one of the preceding embodiments.

In a thirty-fifth embodiment, in a compound, tautomer, a solvate or stereoisomer of the compound or the tautomer, or pharmaceutically acceptable salt of this disclosure, R^{c} is selected from F, methyl, and —OCH₃; all other variables not specifically defined herein are as defined in any one of the preceding embodiments.

In a thirty-sixth embodiment, in a compound, tautomer, a solvate or stereoisomer of the compound or the tautomer, or pharmaceutically acceptable salt of this disclosure, R^{d}, for each occurrence, is independently selected from halogen, C₁-C₆ alkyl, NO₂, CN, and C₁-C₆ alkoxy, wherein the C₁-C₆ alkyl and C₁-C₆ alkoxy of R^{d} are each optionally substituted with 1 to 3 groups selected from halogen; all other variables not specifically defined herein are as defined in any one of the preceding embodiments.

In a thirty-seventh embodiment, in a compound, tautomer, a solvate or stereoisomer of the compound or the tautomer, or pharmaceutically acceptable salt of this disclosure, R^{d} is selected from F, methyl, Cl, NO₂, and —OCH₃; all other variables not specifically defined herein are as defined in any one of the preceding embodiments.

In a thirty-eighth embodiment, in a compound, tautomer, a solvate or stereoisomer of the compound or the tautomer, or pharmaceutically acceptable salt of this disclosure, R^{d} is selected from halogen, C₁-C₂ alkyl, and C₁-C₂ alkoxy; all other variables not specifically defined herein are as defined in any one of the preceding embodiments.

In a thirty-nineth embodiment, in a compound, tautomer, a solvate or stereoisomer of the compound or the tautomer, or pharmaceutically acceptable salt of this disclosure, R^{d} is selected from F, methyl, and —OCH₃; all other variables not specifically defined herein are as defined in any one of the preceding embodiments.

In a fortieth embodiment, in a compound, tautomer, a solvate or stereoisomer of the compound or the tautomer, or pharmaceutically acceptable salt of this disclosure, R^{e}, for each occurrence, is independently selected from halogen, cyano, C₁-C₁₀ alkyl, phenyl, 5- to 7-membered heteroaryl, 3- to 10-membered heterocyclyl, C₃-C₁₀ cycloalkyl, —C(═O)R^{s}, C₂-C₁₀ alkenyl, ═O, ═NR^{p}; —C(═O)OR^{s}, —C(═O)NR^{p}R^{q}, —NR^{p}R^{q}, and —NR^{p}C(═O)R^{s}, wherein
the C₁-C₁₀ alkyl and C₂-C₁₀ alkenyl of R^{e} are each optionally substituted with 1 to 3 groups selected from 5- to 7-membered heteroaryl, C₃-C₁₀ cycloalkyl, 3- to 10-membered heterocyclyl, —OR^{s}, —C(═O)NR^{p}R^{q}, —NR^{p}C(═O)NR^{g}R^{r}, and —NR^{p}R^{q};
the 3- to 10-membered heterocyclyl and C₃-C₁₀ cycloalkyl of R^{e} and the 3- to 10-membered heterocyclyl and C₃-C₁₀ cycloalkyl of the C₁-C₁₀ alkyl and C₂-C₁₀ alkenyl of R^{e}, are each optionally substituted with 1 to 3 groups selected from ═O and C₁-C₆ alkyl optionally substituted with 1 to 3 groups selected from halogen and —OR^{s};
the phenyl and 5- to 7-membered heteroaryl of R^{e} and the 5- to 7-membered heteroaryl of the C₁-C₁₀ alkyl and C₂-C₁₀ alkenyl of R^{e} are each optionally substituted with 1 to 3 groups selected from C₁-C₆ alkyl optionally substituted with 1 to 3 groups selected from halogen and —OR^{s};
R^{p}, R^{q}, and R^{r}, for each occurrence, are independently selected from hydrogen, C₃-C₁₀ cycloalkyl, and C₁-C₁₀ alkyl, wherein the C₃-C₁₀ cycloalkyl and C₁-C₁₀ alkyl of R^{p}, R^{g}, and R^{r} are each optionally substituted with 1 to 3 groups selected from halogen, —OR^{s} and —C(═O)OR^{s};
R^{s}, for each occurrence, is independently selected from H, C₃-C₁₀ cycloalkyl, 3- to 10-membered heterocyclyl, and C₁-C₁₀ alkyl optionally substituted with 1 to 3 groups selected from halogen, C₃-C₁₀ cycloalkyl, 3- to 10-membered heterocyclyl, and C₁-C₆ alkoxy, wherein
the C₃-C₁₀ cycloalkyl and 3- to 10-membered heterocyclyl of R^{s} and the C₃-C₁₀ cycloalkyl and 3- to 10-membered heterocyclyl of the C₁-C₁₀ alkyl of R^{s} are each optionally substituted with 1 to 3 groups of C₁-C₆ alkyl optionally substituted by halogen, and the C₁-C₆ alkoxy of the C₁-C₁₀ alkyl of R^{s} is optionally substituted with 1 to 3 groups of halogen; and
   all other variables not specifically defined herein are as defined in any one of the preceding embodiments.

In a forty-first embodiment, in a compound, tautomer, a solvate or stereoisomer of the compound or the tautomer, or pharmaceutically acceptable salt of this disclosure, R^{e}, for each occurrence, is selected from methyl, ethyl, ═O, —(CH₂)₃OCH₃, —(CH₂)₂OCH₃, —CH₂C(═O)NH₂, —(CH₂)₂C(═O)NH₂, —C(═O)C(CH₃)₃, —C(═O)NHC(CH₃)₃, —(CH₂)₂NHC(═O)NH₂, —(CH₂)₃NHCH₃, —(CH₂)₂NHCH₃, —(CH₂)₃C(═O)NH₂, —(CH₂)₂N(CH₃)₂, —(CH₂)₂OH,

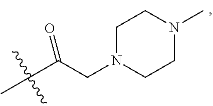 [SMILES: *C(=O)CN1CCN(C)CC1]

—C(═O)CH₃, —(CH₂)₂NH₂,

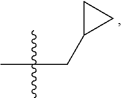 [SMILES: *CC1CC1]

—C(═O)OC(CH₃)₃,

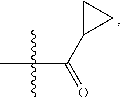 [SMILES: *C(=O)C1CC1]

—C(CH₃)₃, —(CH₂)₂O(CH₂)₂OCH₃, Bn (benzyl), —(CH₂)₃N(CH₃)₂,

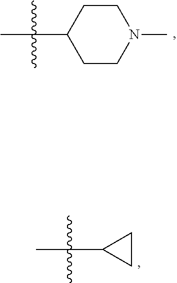

—CH(CH₃)₂,

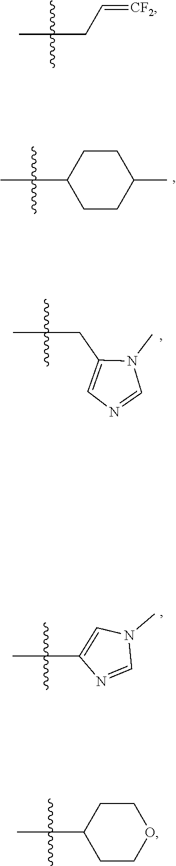

═NH, ═NCH₃, —C(═O)OH, —C(═O)CF₃, —C(═O)NHCH₂CH₃, —NHC(═O)CH₃,

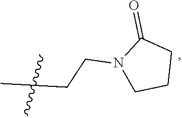 [SMILES: *CCN1CCCC1=O]

—N(CH₃)(CH₂)₂OCH₂OCH₂CH₃, —NH₂, —C(═O)N₂,

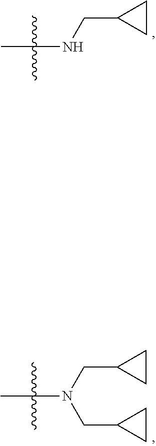 [SMILES: *NCC1CC1 ; *N(CC1CC1)CC1CC1]

—N(CH₃)₂, —NHCH₂CH₃, and

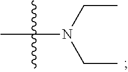 [SMILES: *N(CC)CC]

all other variables not specifically defined herein are as defined in any one of the preceding embodiments.

In a forty-second embodiment, in a compound, tautomer, a solvate or stereoisomer of the compound or the tautomer, or pharmaceutically acceptable salt of this disclosure, R^{e} is selected from ═O, —C(═O)NR^{p}R^{q}, and C₁-C₆ alkyl, wherein the C₁-C₆ alkyl is optionally substituted with 1 to 3 groups selected from C₁-C₃ alkoxy, —C(═O)NR^{p}R^{q}, and —NR^{p}R^{q}, wherein R^{p} and R^{q} are each selected from H and C₁-C₃ alkyl; all other variables not specifically defined herein are as defined in any one of the preceding embodiments.

In a forty-third embodiment, in a compound, tautomer, a solvate or stereoisomer of the compound or the tautomer, or pharmaceutically acceptable salt of this disclosure, R^{e} is selected from C₁-C₂ alkyl, ═O, and —C(═O)NH₂; all other variables not specifically defined herein are as defined in any one of the preceding embodiments.

In a forty-fourth embodiment, in a compound, tautomer, a solvate or stereoisomer of the compound or the tautomer, or pharmaceutically acceptable salt of this disclosure, R^{a} and R^{b} join to form a 5- to 6-membered carbocyclic, heterocyclic, aromatic, or heteroaromatic ring optionally substituted with 1 to 3 groups selected from ═O, C₁-C₆ alkyl, and 5- to 7-membered heterocyclyl, wherein the C₁-C₆ alkyl is optionally substituted with 1 to 3 groups of halogen, and the 5- to 7-membered heterocyclyl is optionally substituted with 1 to 3 groups of C₁-C₃ alkyl optionally substituted with 1 to 3 groups of halogen; all other variables not specifically defined herein are as defined in any one of the preceding embodiments.

In a forty-fifth embodiment, in a compound, tautomer, a solvate or stereoisomer of the compound or the tautomer, or pharmaceutically acceptable salt of this disclosure, R^{a} and R^{b} join to form a structure selected from:

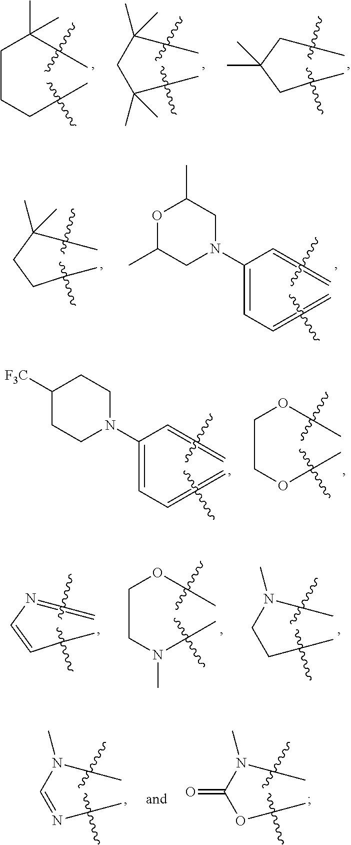

all other variables not specifically defined herein are as defined in any one of the preceding embodiments.

In a forty-sixth embodiment, a compound of the disclosure is a compound of the following structural formulae VIIIa-1, VIIId-1, and VIIIf-1:

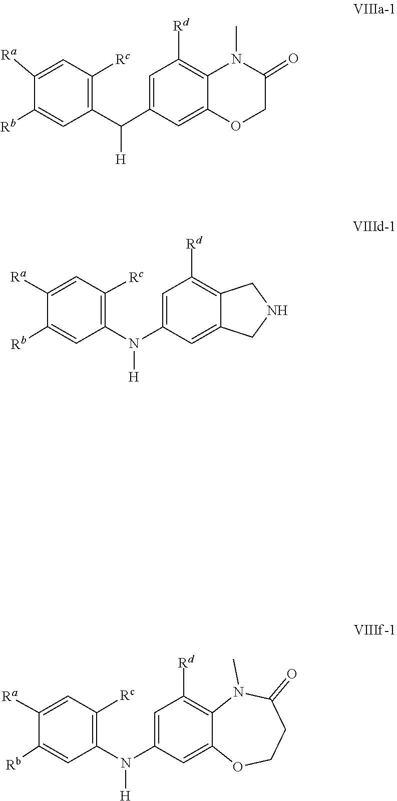

a tautomer thereof, a solvate or stereoisomer of the compound or the tautomer, or a pharmaceutically acceptable salt of the foregoing, wherein:
R^{a} is selected from

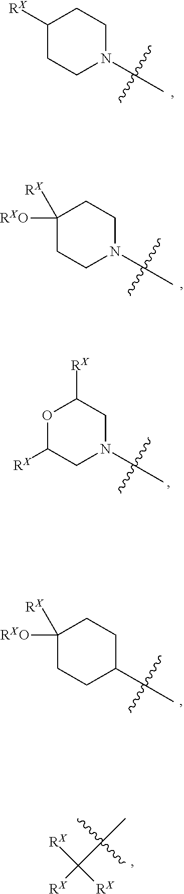

—OR^{x}, and —CH₂R^{x}, wherein R^{x}, for each occurrence, is independently selected from CF₃ and C₁ to C₂ alkyl;
R^{b} is selected from H, halogen, C₁ to C₂ alkyl, and C₁ to C₂ alkoxy;
R^{c} is selected from halogen, C₁ to C₂ alkyl, and C₁ to C₂ alkoxy; and
R^{d} is selected from halogen, C₁ to C₂ alkyl, and C₁ to C₂ alkoxy; and
all other variables not specifically defined herein are as defined in any one of the preceding embodiments.

In certain embodiments, the at least one compound of the disclosure is selected from Compounds 1 to 659 depicted in Table 1, a tautomer thereof, a solvate or stereoisomer of the compound depicted in Table 1 or the tautomer, or a pharmaceutically acceptable salt of the foregoing.

TABLE 1
Compounds 1 to 659

| | |
|---|---|
| 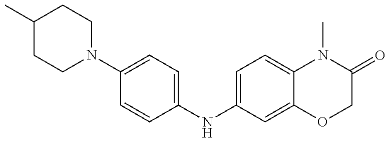 [SMILES: CC1CCN(c2ccc(Nc3ccc4c(c3)OCC(=O)N4C)cc2)CC1] | 1 |
| 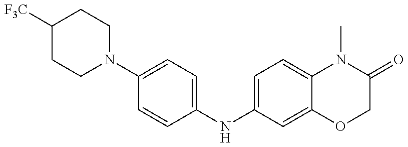 [SMILES: CN1C(=O)COc2cc(Nc3ccc(N4CCC(C(F)(F)F)CC4)cc3)ccc21] | 2 |
| 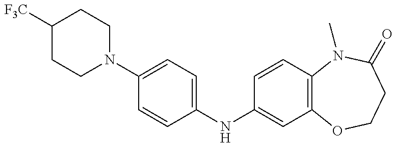 [SMILES: CN1C(=O)CCOc2cc(Nc3ccc(N4CCC(C(F)(F)F)CC4)cc3)ccc21] | 3 |
| 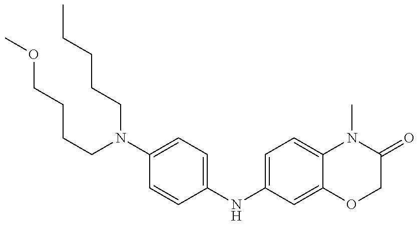 [SMILES: CCCCCN(CCCCOC)c1ccc(Nc2ccc3c(c2)OCC(=O)N3C)cc1] | 4 |
| 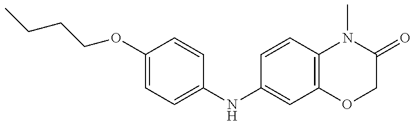 [SMILES: CCCCOc1ccc(Nc2ccc3c(c2)OCC(=O)N3C)cc1] | 5 |
| 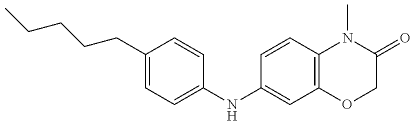 [SMILES: CCCCCc1ccc(Nc2ccc3c(c2)OCC(=O)N3C)cc1] | 6 |
| 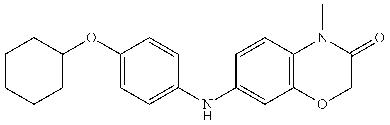 [SMILES: CN1C(=O)COc2cc(Nc3ccc(OC4CCCCC4)cc3)ccc21] | 7 |
| 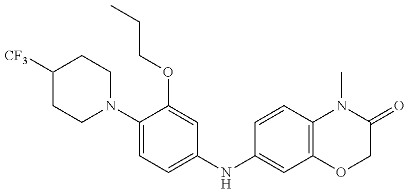 [SMILES: CCCOc1cc(Nc2ccc3c(c2)OCC(=O)N3C)ccc1N1CCC(C(F)(F)F)CC1] | 8 |
| 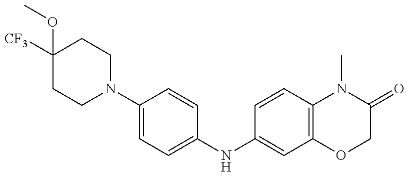 [SMILES: COC1(C(F)(F)F)CCN(c2ccc(Nc3ccc4c(c3)OCC(=O)N4C)cc2)CC1] | 9 |
| 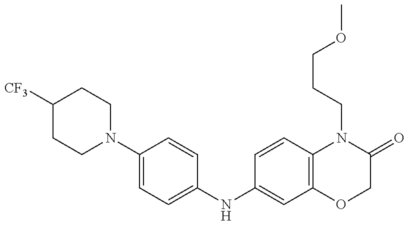 [SMILES: COCCCN1C(=O)COc2cc(Nc3ccc(N4CCC(C(F)(F)F)CC4)cc3)ccc21] | 10 |
| 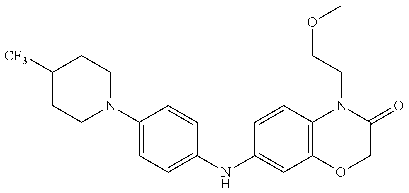 [SMILES: COCCN1C(=O)COc2cc(Nc3ccc(N4CCC(C(F)(F)F)CC4)cc3)ccc21] | 11 |
| 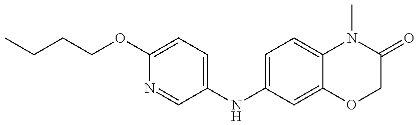 [SMILES: CCCCOc1ccc(Nc2ccc3c(c2)OCC(=O)N3C)cn1] | 12 |
| 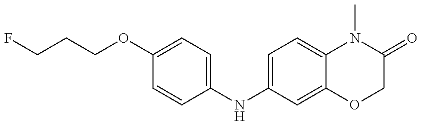 [SMILES: CN1C(=O)COc2cc(Nc3ccc(OCCCF)cc3)ccc21] | 13 |
| 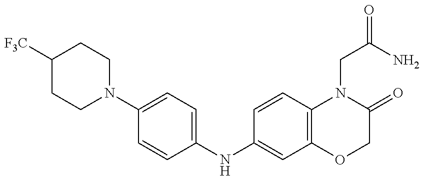 [SMILES: NC(=O)CN1C(=O)COc2cc(Nc3ccc(N4CCC(C(F)(F)F)CC4)cc3)ccc21] | 14 |
| 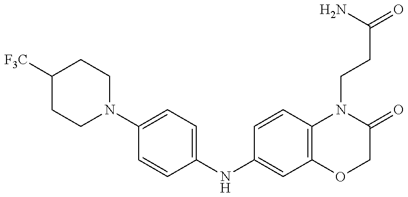 [SMILES: NC(=O)CCN1C(=O)COc2cc(Nc3ccc(N4CCC(C(F)(F)F)CC4)cc3)ccc21] | 15 |
| 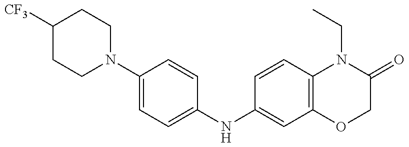 [SMILES: CCN1C(=O)COc2cc(Nc3ccc(N4CCC(C(F)(F)F)CC4)cc3)ccc21] | 16 |
| 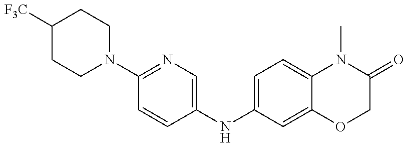 [SMILES: CN1C(=O)COc2cc(Nc3ccc(N4CCC(C(F)(F)F)CC4)nc3)ccc21] | 17 |
| 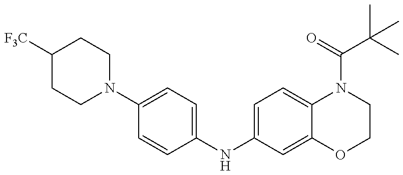 [SMILES: CC(C)(C)C(=O)N1CCOc2cc(Nc3ccc(N4CCC(C(F)(F)F)CC4)cc3)ccc21] | 18 |
| 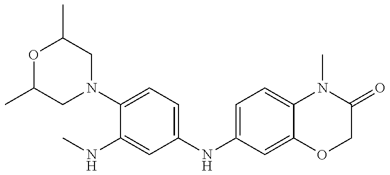 [SMILES: CNc1cc(Nc2ccc3c(c2)OCC(=O)N3C)ccc1N1CC(C)OC(C)C1] | 19 |
| 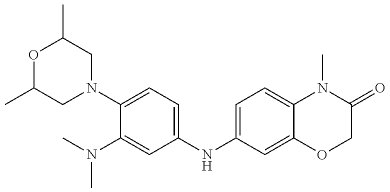 [SMILES: CC1CN(c2ccc(Nc3ccc4c(c3)OCC(=O)N4C)cc2N(C)C)CC(C)O1] | 20 |
| 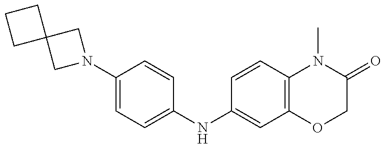 [SMILES: CN1C(=O)COc2cc(Nc3ccc(N4CC5(CCC5)C4)cc3)ccc21] | 21 |
| 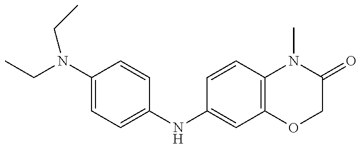 [SMILES: CCN(CC)c1ccc(Nc2ccc3c(c2)OCC(=O)N3C)cc1] | 22 |
| 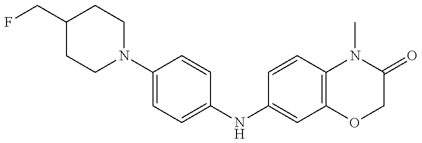 [SMILES: CN1C(=O)COc2cc(Nc3ccc(N4CCC(CF)CC4)cc3)ccc21] | 23 |
| 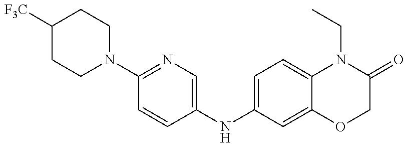 [SMILES: CCN1C(=O)COc2cc(Nc3ccc(N4CCC(C(F)(F)F)CC4)nc3)ccc21] | 24 |
| 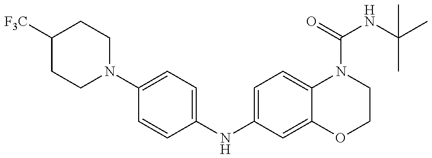 [SMILES: CC(C)(C)NC(=O)N1CCOc2cc(Nc3ccc(N4CCC(C(F)(F)F)CC4)cc3)ccc21] | 25 |
| 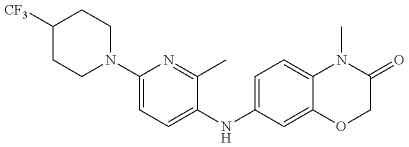 [SMILES: Cc1nc(N2CCC(C(F)(F)F)CC2)ccc1Nc1ccc2c(c1)OCC(=O)N2C] | 26 |
| 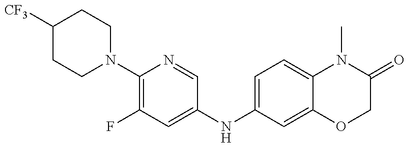 [SMILES: CN1C(=O)COc2cc(Nc3cnc(N4CCC(C(F)(F)F)CC4)c(F)c3)ccc21] | 27 |
| 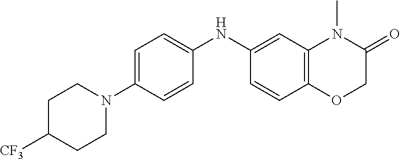 [SMILES: CN1C(=O)COc2ccc(Nc3ccc(N4CCC(C(F)(F)F)CC4)cc3)cc21] | 28 |
| 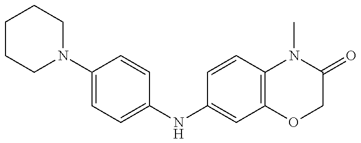 [SMILES: CN1C(=O)COc2cc(Nc3ccc(N4CCCCC4)cc3)ccc21] | 29 |
| 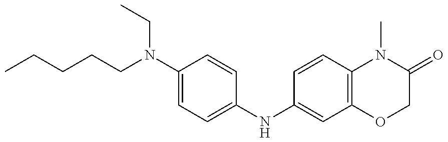 [SMILES: CCCCCN(CC)c1ccc(Nc2ccc3c(c2)OCC(=O)N3C)cc1] | 30 |
| 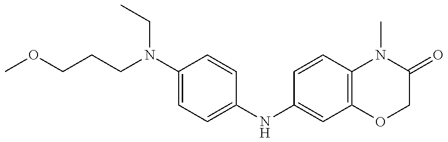 [SMILES: CCN(CCCOC)c1ccc(Nc2ccc3c(c2)OCC(=O)N3C)cc1] | 31 |
| 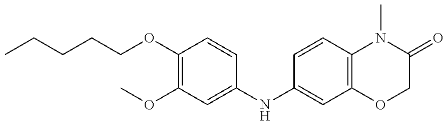 [SMILES: CCCCCOc1ccc(Nc2ccc3c(c2)OCC(=O)N3C)cc1OC] | 32 |
| 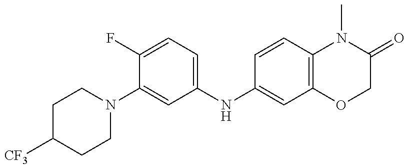 [SMILES: CN1C(=O)COc2cc(Nc3ccc(F)c(N4CCC(C(F)(F)F)CC4)c3)ccc21] | 33 |
| 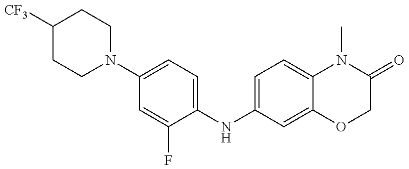 [SMILES: CN1C(=O)COc2cc(Nc3ccc(N4CCC(C(F)(F)F)CC4)cc3F)ccc21] | 34 |
| 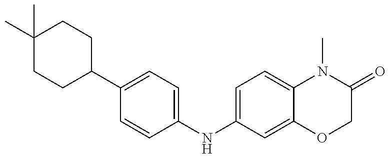 [SMILES: CN1C(=O)COc2cc(Nc3ccc(C4CCC(C)(C)CC4)cc3)ccc21] | 35 |
| 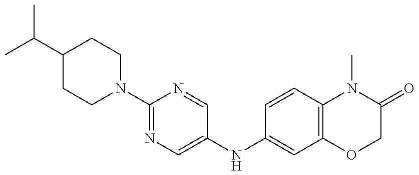 [SMILES: CC(C)C1CCN(c2ncc(Nc3ccc4c(c3)OCC(=O)N4C)cn2)CC1] | 36 |
| 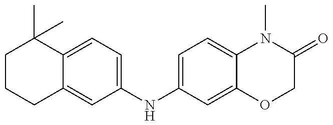 [SMILES: CN1C(=O)COc2cc(Nc3ccc4c(c3)CCCC4(C)C)ccc21] | 37 |
| 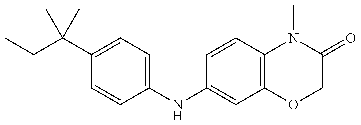 [SMILES: CCC(C)(C)c1ccc(Nc2ccc3c(c2)OCC(=O)N3C)cc1] | 38 |
| 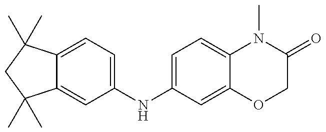 [SMILES: CN1C(=O)COc2cc(Nc3ccc4c(c3)C(C)(C)CC4(C)C)ccc21] | 39 |
| 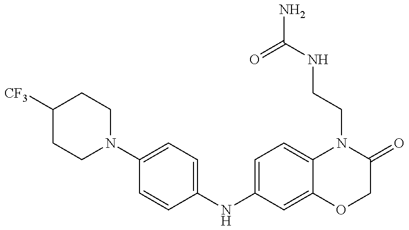 [SMILES: NC(=O)NCCN1C(=O)COc2cc(Nc3ccc(N4CCC(C(F)(F)F)CC4)cc3)ccc21] | 40 |
| 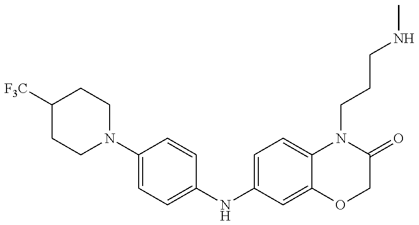 [SMILES: CNCCCN1C(=O)COc2cc(Nc3ccc(N4CCC(C(F)(F)F)CC4)cc3)ccc21] | 41 |
| 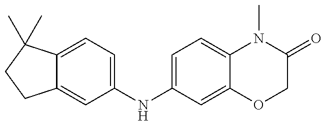 [SMILES: CN1C(=O)COc2cc(Nc3ccc4c(c3)CCC4(C)C)ccc21] | 42 |
| 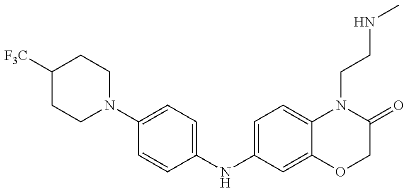 [SMILES: CNCCN1C(=O)COc2cc(Nc3ccc(N4CCC(C(F)(F)F)CC4)cc3)ccc21] | 43 |
| 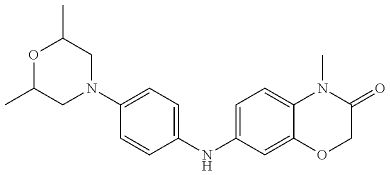 [SMILES: CC1CN(c2ccc(Nc3ccc4c(c3)OCC(=O)N4C)cc2)CC(C)O1] | 44 |
| 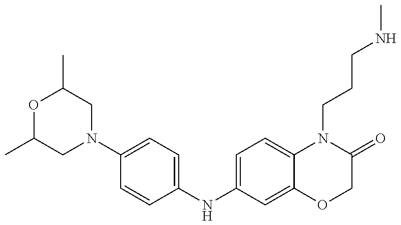 | 45 |
| 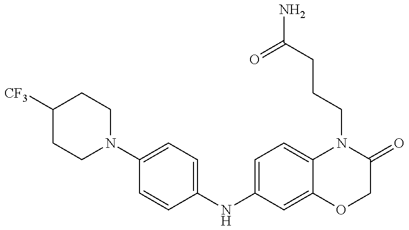 [SMILES: NC(=O)CCCN1C(=O)COc2cc(Nc3ccc(N4CCC(C(F)(F)F)CC4)cc3)ccc21] | 46 |
| 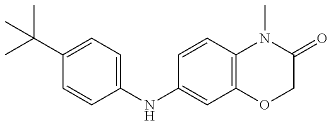 [SMILES: CN1C(=O)COc2cc(Nc3ccc(C(C)(C)C)cc3)ccc21] | 47 |
| 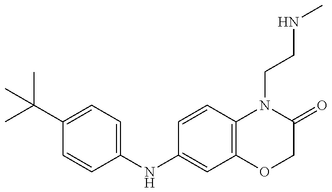 [SMILES: CNCCN1C(=O)COc2cc(Nc3ccc(C(C)(C)C)cc3)ccc21] | 48 |
| 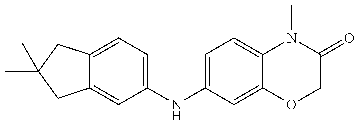 [SMILES: CN1C(=O)COc2cc(Nc3ccc4c(c3)CC(C)(C)C4)ccc21] | 49 |
| 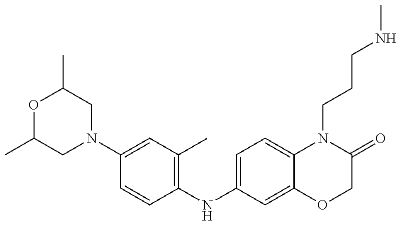 | 50 |
| 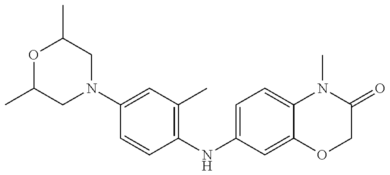 [SMILES: Cc1cc(N2CC(C)OC(C)C2)ccc1Nc1ccc2c(c1)OCC(=O)N2C] | 51 |
| 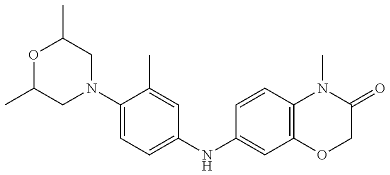 [SMILES: Cc1cc(Nc2ccc3c(c2)OCC(=O)N3C)ccc1N1CC(C)OC(C)C1] | 52 |
| 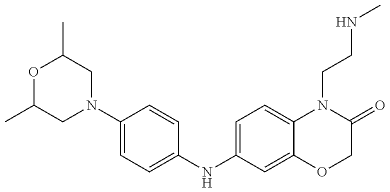 | 53 |
| 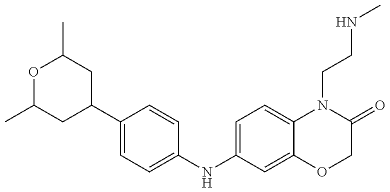 | 54 |
| 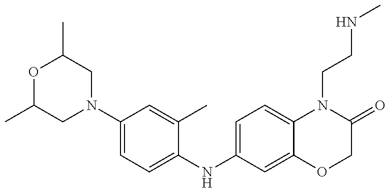 | 55 |
| 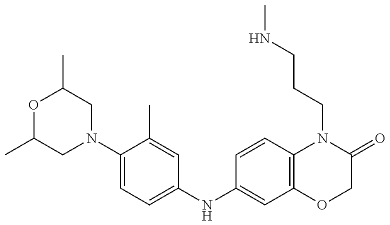 [SMILES: CNCCCN1C(=O)COc2cc(Nc3ccc(N4CC(C)OC(C)C4)c(C)c3)ccc21] | 56 |
| 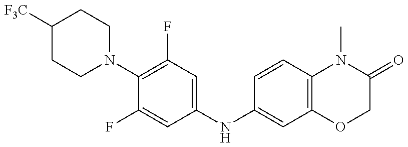 [SMILES: CN1C(=O)COc2cc(Nc3cc(F)c(N4CCC(C(F)(F)F)CC4)c(F)c3)ccc21] | 57 |
| 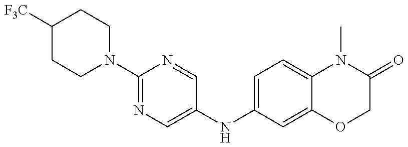 [SMILES: CN1C(=O)COc2cc(Nc3cnc(N4CCC(C(F)(F)F)CC4)nc3)ccc21] | 58 |
| 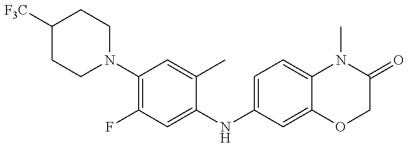 [SMILES: Cc1cc(N2CCC(C(F)(F)F)CC2)c(F)cc1Nc1ccc2c(c1)OCC(=O)N2C] | 59 |
| 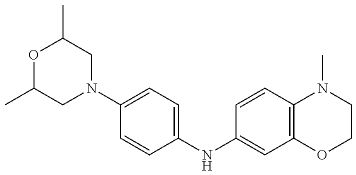 [SMILES: CC1CN(c2ccc(Nc3ccc4c(c3)OCCN4C)cc2)CC(C)O1] | 60 |
| 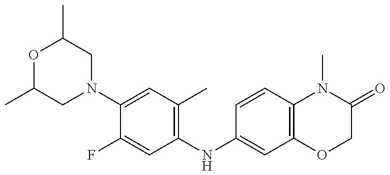 [SMILES: Cc1cc(N2CC(C)OC(C)C2)c(F)cc1Nc1ccc2c(c1)OCC(=O)N2C] | 61 |
| 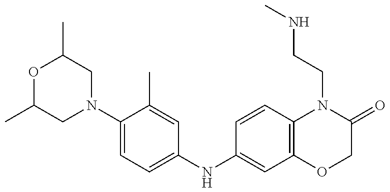 [SMILES: CNCCN1C(=O)COc2cc(Nc3ccc(N4CC(C)OC(C)C4)c(C)c3)ccc21] | 62 |
| 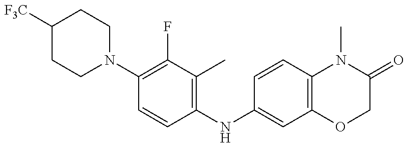 [SMILES: Cc1c(Nc2ccc3c(c2)OCC(=O)N3C)ccc(N2CCC(C(F)(F)F)CC2)c1F] | 63 |
| 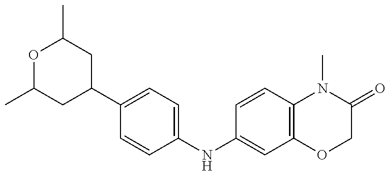 [SMILES: CC1CC(c2ccc(Nc3ccc4c(c3)OCC(=O)N4C)cc2)CC(C)O1] | 64 |
| 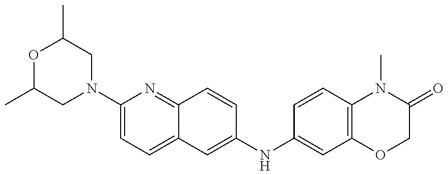 [SMILES: CC1CN(c2ccc3cc(Nc4ccc5c(c4)OCC(=O)N5C)ccc3n2)CC(C)O1] | 65 |
| 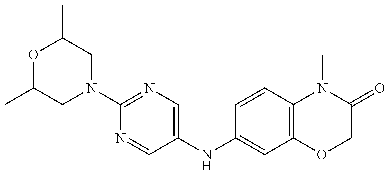 [SMILES: CC1CN(c2ncc(Nc3ccc4c(c3)OCC(=O)N4C)cn2)CC(C)O1] | 66 |
| 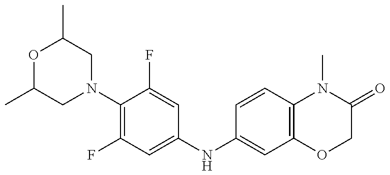 [SMILES: CC1CN(c2c(F)cc(Nc3ccc4c(c3)OCC(=O)N4C)cc2F)CC(C)O1] | 67 |
| 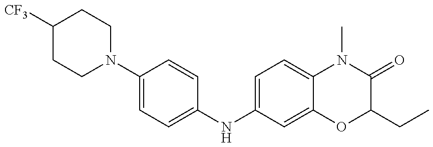 [SMILES: CCC1Oc2cc(Nc3ccc(N4CCC(C(F)(F)F)CC4)cc3)ccc2N(C)C1=O] | 68 |
| 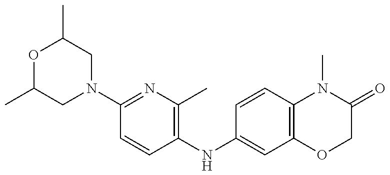 [SMILES: Cc1nc(N2CC(C)OC(C)C2)ccc1Nc1ccc2c(c1)OCC(=O)N2C] | 69 |
| 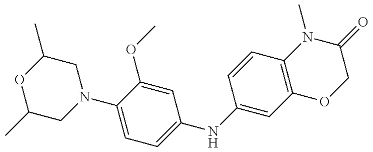 [SMILES: COc1cc(Nc2ccc3c(c2)OCC(=O)N3C)ccc1N1CC(C)OC(C)C1] | 70 |
| 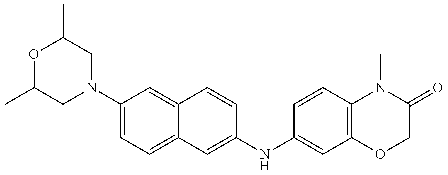 [SMILES: CC1CN(c2ccc3cc(Nc4ccc5c(c4)OCC(=O)N5C)ccc3c2)CC(C)O1] | 71 |
| 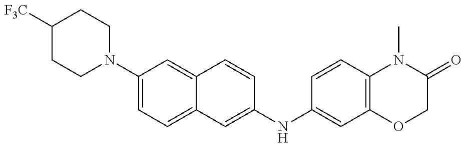 [SMILES: CN1C(=O)COc2cc(Nc3ccc4cc(N5CCC(C(F)(F)F)CC5)ccc4c3)ccc21] | 72 |
| 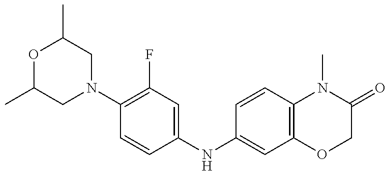 [SMILES: CC1CN(c2ccc(Nc3ccc4c(c3)OCC(=O)N4C)cc2F)CC(C)O1] | 73 |
| 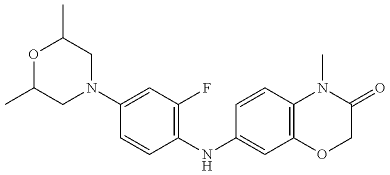 [SMILES: CC1CN(c2ccc(Nc3ccc4c(c3)OCC(=O)N4C)c(F)c2)CC(C)O1] | 74 |
| 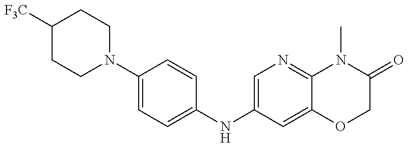 [SMILES: CN1C(=O)COc2cc(Nc3ccc(N4CCC(C(F)(F)F)CC4)cc3)cnc21] | 75 |
| 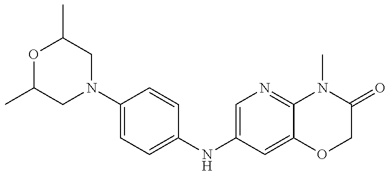 [SMILES: CC1CN(c2ccc(Nc3cnc4c(c3)OCC(=O)N4C)cc2)CC(C)O1] | 76 |
| 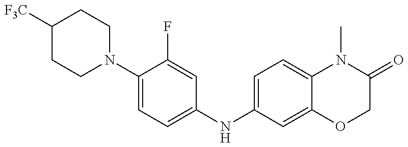 [SMILES: CN1C(=O)COc2cc(Nc3ccc(N4CCC(C(F)(F)F)CC4)c(F)c3)ccc21] | 77 |
| 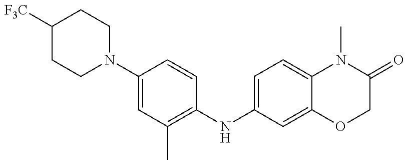 [SMILES: Cc1cc(N2CCC(C(F)(F)F)CC2)ccc1Nc1ccc2c(c1)OCC(=O)N2C] | 78 |
| 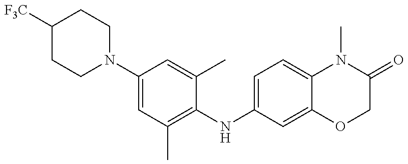 [SMILES: Cc1cc(N2CCC(C(F)(F)F)CC2)cc(C)c1Nc1ccc2c(c1)OCC(=O)N2C] | 79 |
| 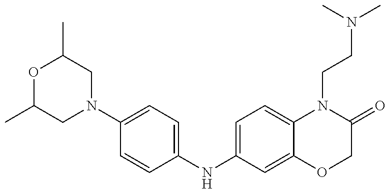 | 80 |
| 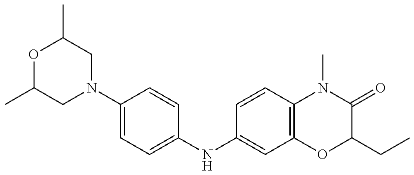 [SMILES: CCC1Oc2cc(Nc3ccc(N4CC(C)OC(C)C4)cc3)ccc2N(C)C1=O] | 81 |
| 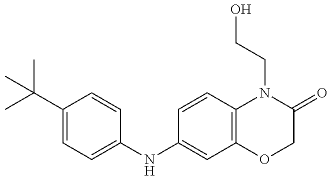 [SMILES: CC(C)(C)c1ccc(Nc2ccc3c(c2)OCC(=O)N3CCO)cc1] | 82 |
| 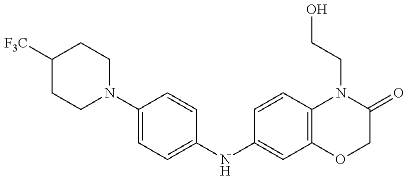 [SMILES: O=C1COc2cc(Nc3ccc(N4CCC(C(F)(F)F)CC4)cc3)ccc2N1CCO] | 83 |
| 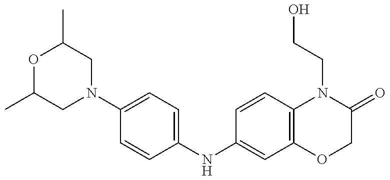 [SMILES: CC1CN(c2ccc(Nc3ccc4c(c3)OCC(=O)N4CCO)cc2)CC(C)O1] | 84 |
| 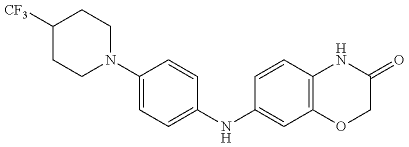 [SMILES: O=C1COc2cc(Nc3ccc(N4CCC(C(F)(F)F)CC4)cc3)ccc2N1] | 85 |
| 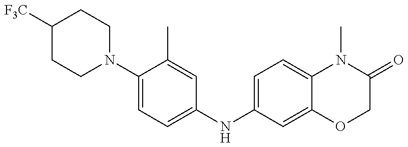 [SMILES: Cc1cc(Nc2ccc3c(c2)OCC(=O)N3C)ccc1N1CCC(C(F)(F)F)CC1] | 86 |
| 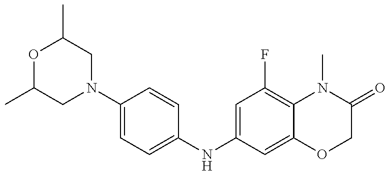 [SMILES: CC1CN(c2ccc(Nc3cc(F)c4c(c3)OCC(=O)N4C)cc2)CC(C)O1] | 87 |
| 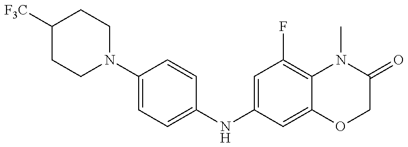 [SMILES: CN1C(=O)COc2cc(Nc3ccc(N4CCC(C(F)(F)F)CC4)cc3)cc(F)c21] | 88 |
| 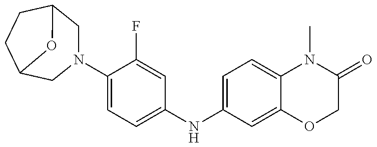 [SMILES: CN1C(=O)COc2cc(Nc3ccc(N4CC5CCC(C4)O5)c(F)c3)ccc21] | 89 |
| 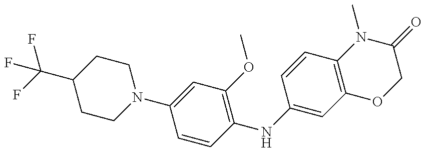 [SMILES: COc1cc(N2CCC(C(F)(F)F)CC2)ccc1Nc1ccc2c(c1)OCC(=O)N2C] | 90 |
| 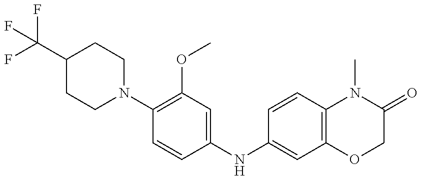 [SMILES: COc1cc(Nc2ccc3c(c2)OCC(=O)N3C)ccc1N1CCC(C(F)(F)F)CC1] | 91 |
| 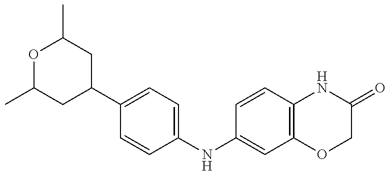 [SMILES: CC1CC(c2ccc(Nc3ccc4c(c3)OCC(=O)N4)cc2)CC(C)O1] | 92 |
| 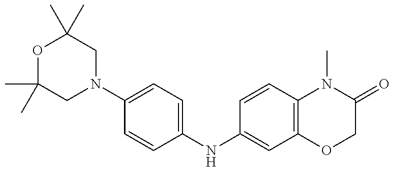 [SMILES: CN1C(=O)COc2cc(Nc3ccc(N4CC(C)(C)OC(C)(C)C4)cc3)ccc21] | 93 |
| 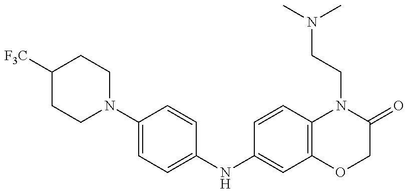 [SMILES: CN(C)CCN1C(=O)COc2cc(Nc3ccc(N4CCC(C(F)(F)F)CC4)cc3)ccc21] | 94 |
| 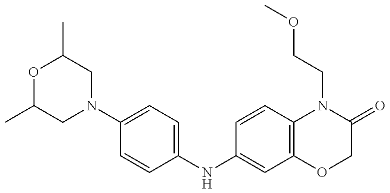 [SMILES: COCCN1C(=O)COc2cc(Nc3ccc(N4CC(C)OC(C)C4)cc3)ccc21] | 95 |
| 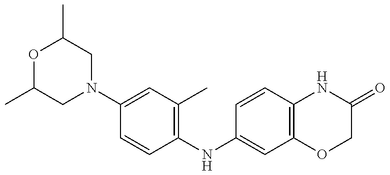 [SMILES: Cc1cc(N2CC(C)OC(C)C2)ccc1Nc1ccc2c(c1)OCC(=O)N2] | 96 |
| 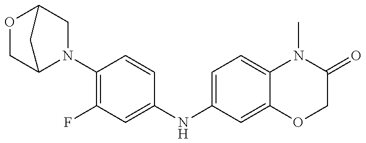 [SMILES: CN1C(=O)COc2cc(Nc3ccc(N4CC5CC4CO5)c(F)c3)ccc21] | 97 |
| 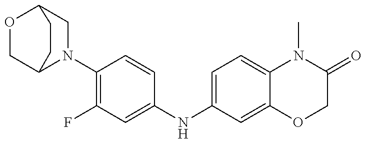 [SMILES: CN1C(=O)COc2cc(Nc3ccc(N4CC5CCC4CO5)c(F)c3)ccc21] | 98 |
| 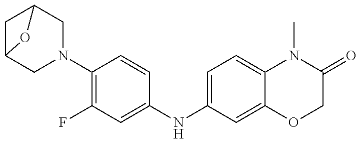 [SMILES: CN1C(=O)COc2cc(Nc3ccc(N4CC5CC(C4)O5)c(F)c3)ccc21] | 99 |
| 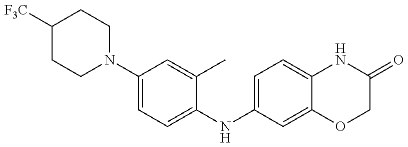 [SMILES: Cc1cc(N2CCC(C(F)(F)F)CC2)ccc1Nc1ccc2c(c1)OCC(=O)N2] | 100 |
| 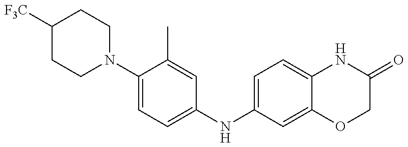 [SMILES: Cc1cc(Nc2ccc3c(c2)OCC(=O)N3)ccc1N1CCC(C(F)(F)F)CC1] | 101 |
| 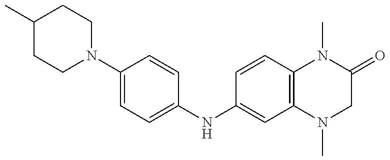 [SMILES: CC1CCN(c2ccc(Nc3ccc4c(c3)N(C)CC(=O)N4C)cc2)CC1] | 102 |
| 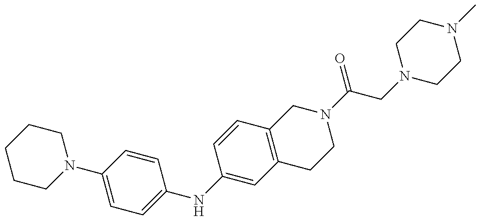 [SMILES: CN1CCN(CC(=O)N2CCc3cc(Nc4ccc(N5CCCCC5)cc4)ccc3C2)CC1] | 103 |
| 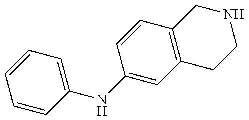 [SMILES: c1ccc(Nc2ccc3c(c2)CCNC3)cc1] | 104 |
| 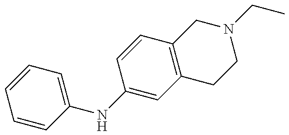 [SMILES: CCN1CCc2cc(Nc3ccccc3)ccc2C1] | 105 |
| 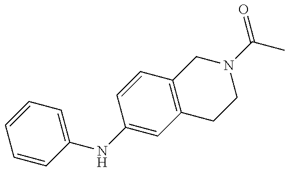 [SMILES: CC(=O)N1CCc2cc(Nc3ccccc3)ccc2C1] | 106 |
| 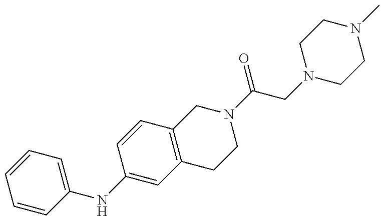 [SMILES: CN1CCN(CC(=O)N2CCc3cc(Nc4ccccc4)ccc3C2)CC1] | 107 |
| 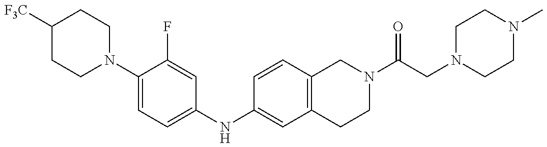 [SMILES: CN1CCN(CC(=O)N2CCc3cc(Nc4ccc(N5CCC(C(F)(F)F)CC5)c(F)c4)ccc3C2)CC1] | 108 |
| 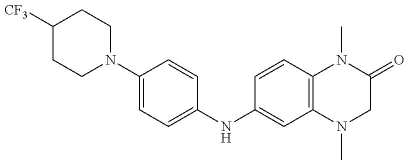 [SMILES: CN1CC(=O)N(C)c2ccc(Nc3ccc(N4CCC(C(F)(F)F)CC4)cc3)cc21] | 109 |
| 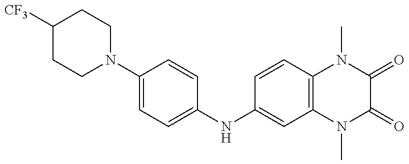 [SMILES: Cn1c(=O)c(=O)n(C)c2cc(Nc3ccc(N4CCC(C(F)(F)F)CC4)cc3)ccc21] | 110 |
| 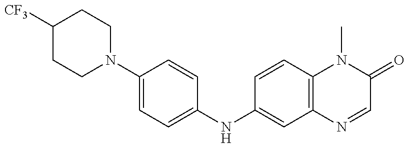 [SMILES: Cn1c(=O)cnc2cc(Nc3ccc(N4CCC(C(F)(F)F)CC4)cc3)ccc21] | 111 |
| 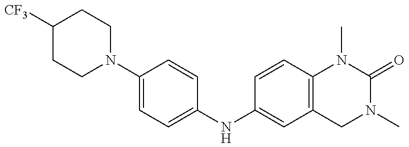 [SMILES: CN1Cc2cc(Nc3ccc(N4CCC(C(F)(F)F)CC4)cc3)ccc2N(C)C1=O] | 112 |
| 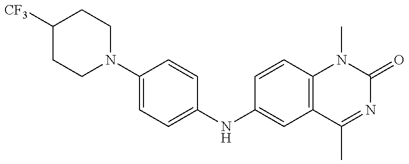 [SMILES: Cc1nc(=O)n(C)c2ccc(Nc3ccc(N4CCC(C(F)(F)F)CC4)cc3)cc12] | 113 |
| 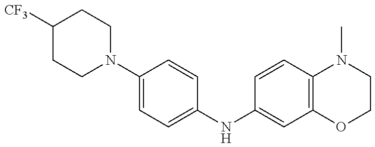 [SMILES: CN1CCOc2cc(Nc3ccc(N4CCC(C(F)(F)F)CC4)cc3)ccc21] | 114 |
| 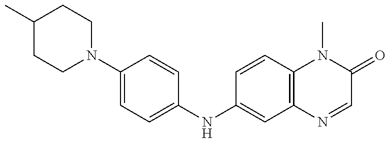 [SMILES: CC1CCN(c2ccc(Nc3ccc4c(c3)ncc(=O)n4C)cc2)CC1] | 115 |
| 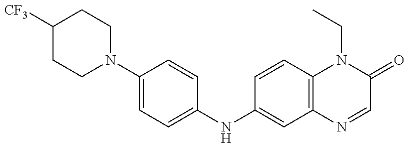 [SMILES: CCn1c(=O)cnc2cc(Nc3ccc(N4CCC(C(F)(F)F)CC4)cc3)ccc21] | 116 |
| 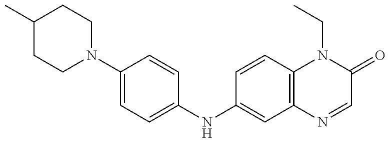 [SMILES: CCn1c(=O)cnc2cc(Nc3ccc(N4CCC(C)CC4)cc3)ccc21] | 117 |
| 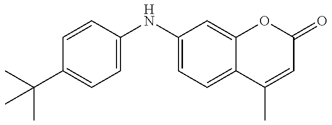 [SMILES: Cc1cc(=O)oc2cc(Nc3ccc(C(C)(C)C)cc3)ccc12] | 118 |
| 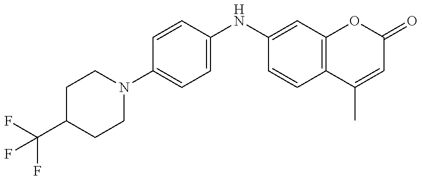 [SMILES: Cc1cc(=O)oc2cc(Nc3ccc(N4CCC(C(F)(F)F)CC4)cc3)ccc12] | 119 |
| 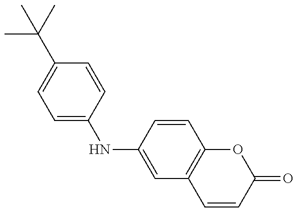 [SMILES: CC(C)(C)c1ccc(Nc2ccc3oc(=O)ccc3c2)cc1] | 120 |
| 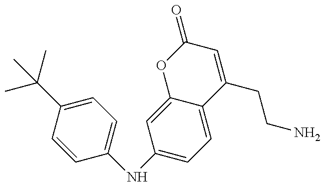 [SMILES: CC(C)(C)c1ccc(Nc2ccc3c(CCN)cc(=O)oc3c2)cc1] | 121 |
| 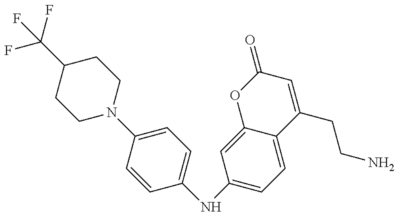 [SMILES: NCCc1cc(=O)oc2cc(Nc3ccc(N4CCC(C(F)(F)F)CC4)cc3)ccc12] | 122 |
| 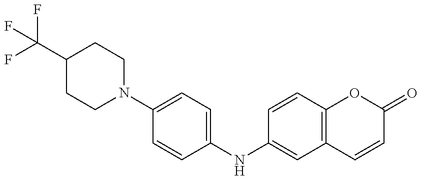 [SMILES: O=c1ccc2cc(Nc3ccc(N4CCC(C(F)(F)F)CC4)cc3)ccc2o1] | 123 |
| 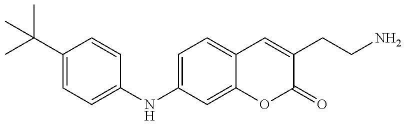 [SMILES: CC(C)(C)c1ccc(Nc2ccc3cc(CCN)c(=O)oc3c2)cc1] | 124 |
| 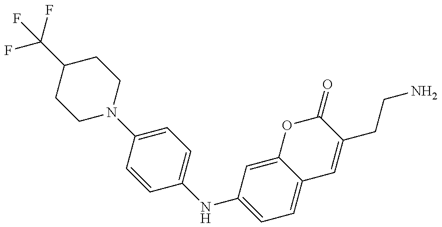 [SMILES: NCCc1cc2ccc(Nc3ccc(N4CCC(C(F)(F)F)CC4)cc3)cc2oc1=O] | 125 |
| 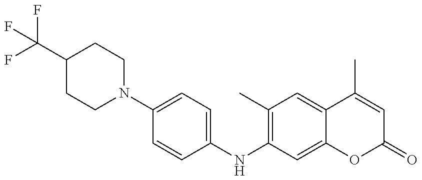 [SMILES: Cc1cc2c(C)cc(=O)oc2cc1Nc1ccc(N2CCC(C(F)(F)F)CC2)cc1] | 126 |
| 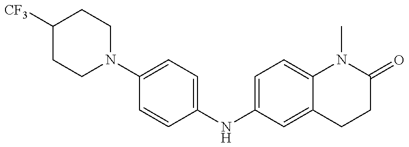 [SMILES: CN1C(=O)CCc2cc(Nc3ccc(N4CCC(C(F)(F)F)CC4)cc3)ccc21] | 127 |
| 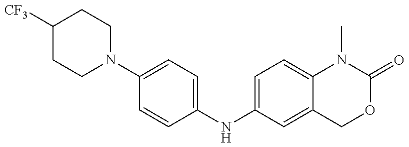 [SMILES: CN1C(=O)OCc2cc(Nc3ccc(N4CCC(C(F)(F)F)CC4)cc3)ccc21] | 128 |
| 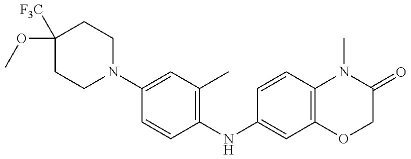 [SMILES: COC1(C(F)(F)F)CCN(c2ccc(Nc3ccc4c(c3)OCC(=O)N4C)c(C)c2)CC1] | 129 |
| 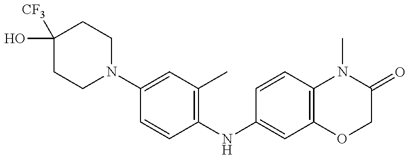 [SMILES: Cc1cc(N2CCC(O)(C(F)(F)F)CC2)ccc1Nc1ccc2c(c1)OCC(=O)N2C] | 130 |
| 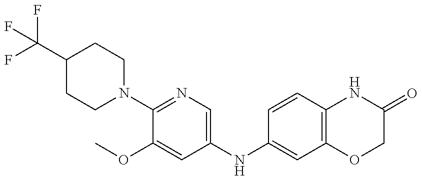 [SMILES: COc1cc(Nc2ccc3c(c2)OCC(=O)N3)cnc1N1CCC(C(F)(F)F)CC1] | 131 |
| 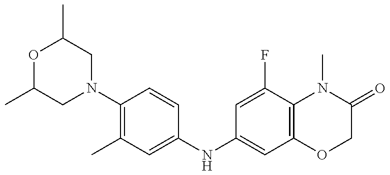 [SMILES: Cc1cc(Nc2cc(F)c3c(c2)OCC(=O)N3C)ccc1N1CC(C)OC(C)C1] | 132 |
| 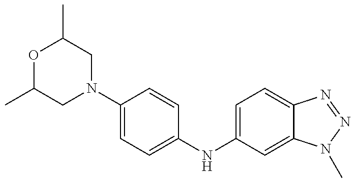 [SMILES: CC1CN(c2ccc(Nc3ccc4nnn(C)c4c3)cc2)CC(C)O1] | 133 |
| 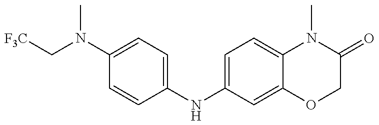 [SMILES: CN(CC(F)(F)F)c1ccc(Nc2ccc3c(c2)OCC(=O)N3C)cc1] | 134 |
| 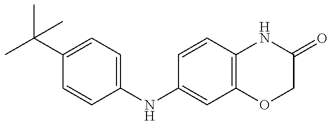 [SMILES: CC(C)(C)c1ccc(Nc2ccc3c(c2)OCC(=O)N3)cc1] | 135 |
| 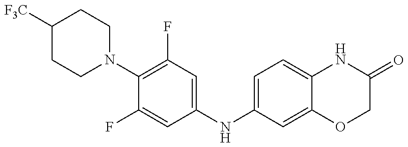 [SMILES: O=C1COc2cc(Nc3cc(F)c(N4CCC(C(F)(F)F)CC4)c(F)c3)ccc2N1] | 136 |
| 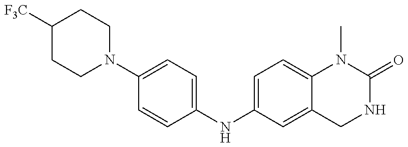 [SMILES: CN1C(=O)NCc2cc(Nc3ccc(N4CCC(C(F)(F)F)CC4)cc3)ccc21] | 137 |
| 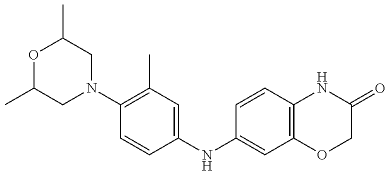 [SMILES: Cc1cc(Nc2ccc3c(c2)OCC(=O)N3)ccc1N1CC(C)OC(C)C1] | 138 |
| 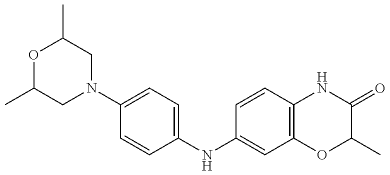 | 139 |
| 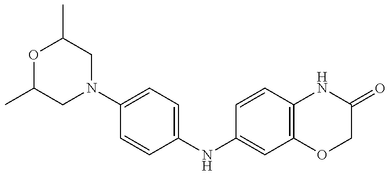 [SMILES: CC1CN(c2ccc(Nc3ccc4c(c3)OCC(=O)N4)cc2)CC(C)O1] | 140 |
| 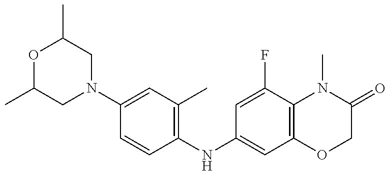 [SMILES: Cc1cc(N2CC(C)OC(C)C2)ccc1Nc1cc(F)c2c(c1)OCC(=O)N2C] | 141 |
| 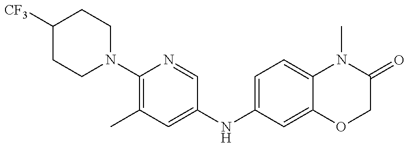 [SMILES: Cc1cc(Nc2ccc3c(c2)OCC(=O)N3C)cnc1N1CCC(C(F)(F)F)CC1] | 142 |
| 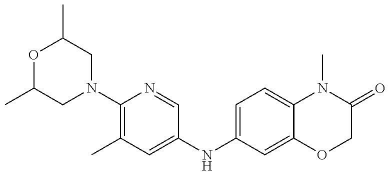 [SMILES: Cc1cc(Nc2ccc3c(c2)OCC(=O)N3C)cnc1N1CC(C)OC(C)C1] | 143 |
| 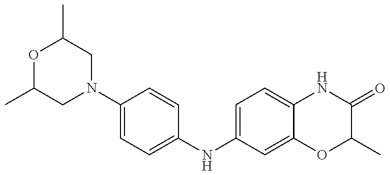 | 144 |
| 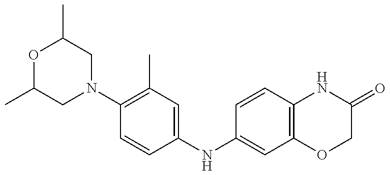 [SMILES: Cc1cc(Nc2ccc3c(c2)OCC(=O)N3)ccc1N1CC(C)OC(C)C1] | 145 |
| 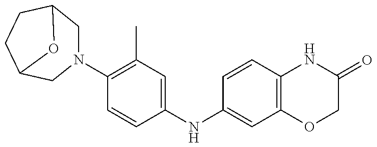 [SMILES: Cc1cc(Nc2ccc3c(c2)OCC(=O)N3)ccc1N1CC2CCC(C1)O2] | 146 |
| 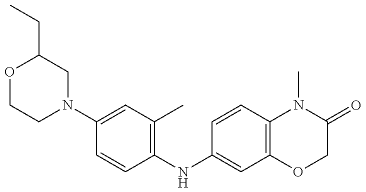 [SMILES: CCC1CN(c2ccc(Nc3ccc4c(c3)OCC(=O)N4C)c(C)c2)CCO1] | 147 |
| 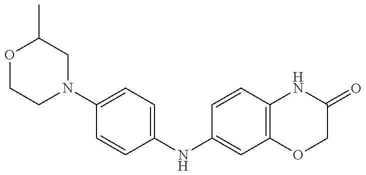 [SMILES: CC1CN(c2ccc(Nc3ccc4c(c3)OCC(=O)N4)cc2)CCO1] | 148 |
| 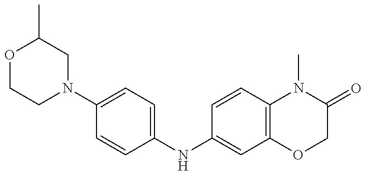 [SMILES: CC1CN(c2ccc(Nc3ccc4c(c3)OCC(=O)N4C)cc2)CCO1] | 149 |
| 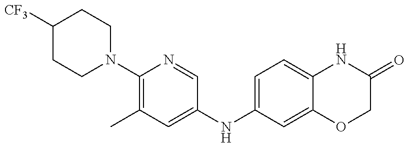 [SMILES: Cc1cc(Nc2ccc3c(c2)OCC(=O)N3)cnc1N1CCC(C(F)(F)F)CC1] | 150 |
| 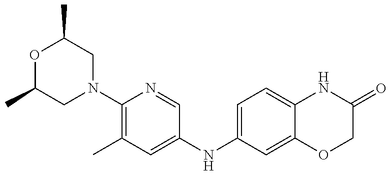 [SMILES: Cc1cc(Nc2ccc3c(c2)OCC(=O)N3)cnc1N1C[C@@H](C)O[C@@H](C)C1] | 151 |
| 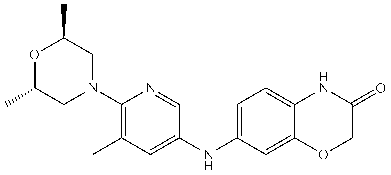 [SMILES: Cc1cc(Nc2ccc3c(c2)OCC(=O)N3)cnc1N1C[C@H](C)O[C@@H](C)C1] | 152 |
| 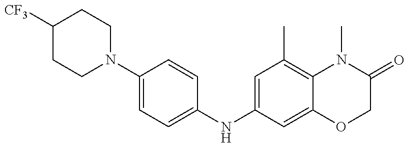 [SMILES: Cc1cc(Nc2ccc(N3CCC(C(F)(F)F)CC3)cc2)cc2c1N(C)C(=O)CO2] | 153 |
| 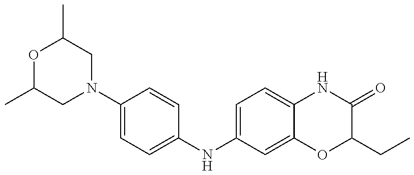 [SMILES: CCC1Oc2cc(Nc3ccc(N4CC(C)OC(C)C4)cc3)ccc2NC1=O] | 154 |
| 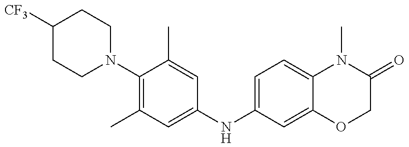 [SMILES: Cc1cc(Nc2ccc3c(c2)OCC(=O)N3C)cc(C)c1N1CCC(C(F)(F)F)CC1] | 155 |
| 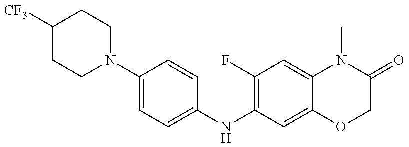 [SMILES: CN1C(=O)COc2cc(Nc3ccc(N4CCC(C(F)(F)F)CC4)cc3)c(F)cc21] | 156 |
| 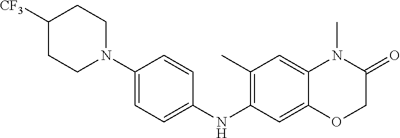 [SMILES: Cc1cc2c(cc1Nc1ccc(N3CCC(C(F)(F)F)CC3)cc1)OCC(=O)N2C] | 157 |
| 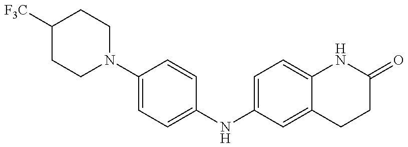 [SMILES: O=C1CCc2cc(Nc3ccc(N4CCC(C(F)(F)F)CC4)cc3)ccc2N1] | 158 |
| 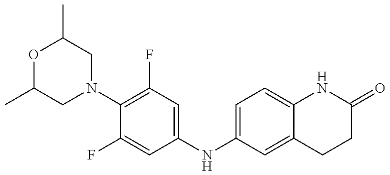 [SMILES: CC1CN(c2c(F)cc(Nc3ccc4c(c3)CCC(=O)N4)cc2F)CC(C)O1] | 159 |
| 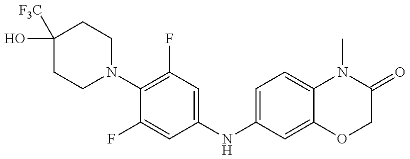 [SMILES: CN1C(=O)COc2cc(Nc3cc(F)c(N4CCC(O)(C(F)(F)F)CC4)c(F)c3)ccc21] | 160 |
| 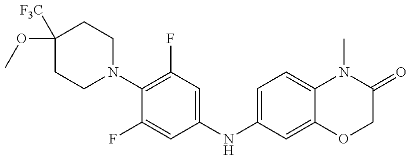 [SMILES: COC1(C(F)(F)F)CCN(c2c(F)cc(Nc3ccc4c(c3)OCC(=O)N4C)cc2F)CC1] | 161 |
| 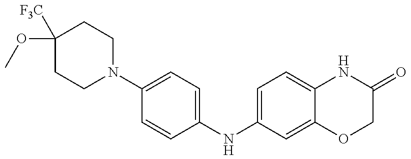 [SMILES: COC1(C(F)(F)F)CCN(c2ccc(Nc3ccc4c(c3)OCC(=O)N4)cc2)CC1] | 162 |
| 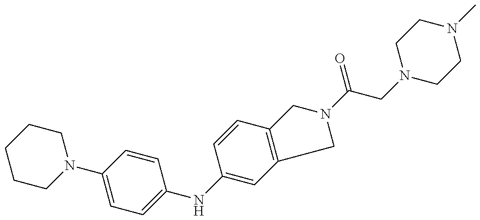 [SMILES: CN1CCN(CC(=O)N2Cc3ccc(Nc4ccc(N5CCCCC5)cc4)cc3C2)CC1] | 163 |
| 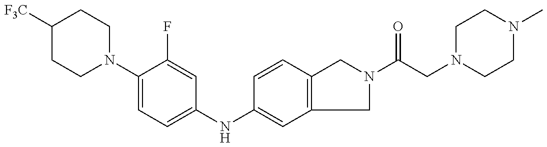 [SMILES: CN1CCN(CC(=O)N2Cc3ccc(Nc4ccc(N5CCC(C(F)(F)F)CC5)c(F)c4)cc3C2)CC1] | 164 |
| 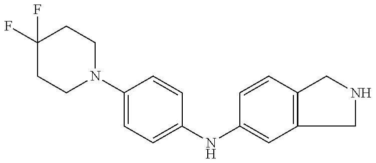 [SMILES: FC1(F)CCN(c2ccc(Nc3ccc4c(c3)CNC4)cc2)CC1] | 165 |
| 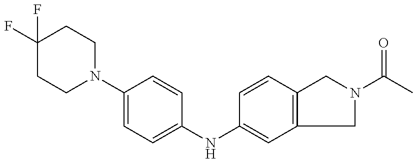 [SMILES: CC(=O)N1Cc2ccc(Nc3ccc(N4CCC(F)(F)CC4)cc3)cc2C1] | 166 |
| 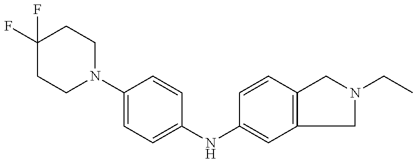 [SMILES: CCN1Cc2ccc(Nc3ccc(N4CCC(F)(F)CC4)cc3)cc2C1] | 167 |
| 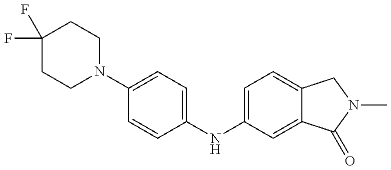 [SMILES: CN1Cc2ccc(Nc3ccc(N4CCC(F)(F)CC4)cc3)cc2C1=O] | 168 |
| 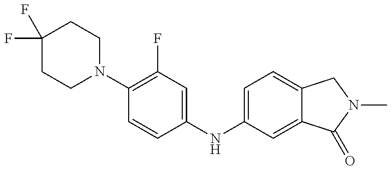 [SMILES: CN1Cc2ccc(Nc3ccc(N4CCC(F)(F)CC4)c(F)c3)cc2C1=O] | 169 |
| 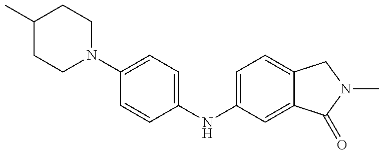 | 170 |
| 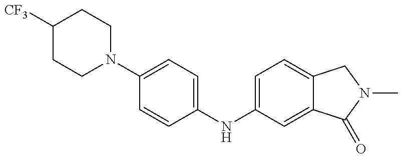 [SMILES: CN1Cc2ccc(Nc3ccc(N4CCC(C(F)(F)F)CC4)cc3)cc2C1=O] | 171 |
| 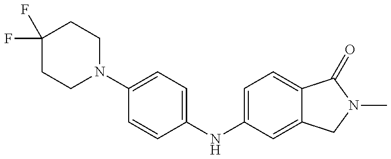 [SMILES: CN1Cc2cc(Nc3ccc(N4CCC(F)(F)CC4)cc3)ccc2C1=O] | 172 |
| 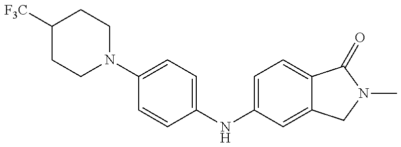 [SMILES: CN1Cc2cc(Nc3ccc(N4CCC(C(F)(F)F)CC4)cc3)ccc2C1=O] | 173 |
| 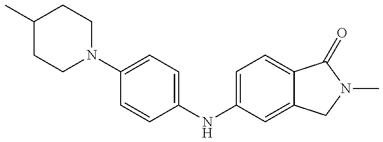 | 174 |
| 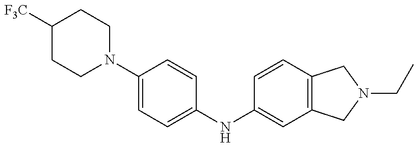 [SMILES: CCN1Cc2ccc(Nc3ccc(N4CCC(C(F)(F)F)CC4)cc3)cc2C1] | 175 |
| 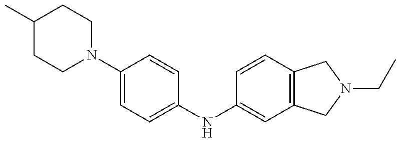 [SMILES: CCN1Cc2ccc(Nc3ccc(N4CCC(C)CC4)cc3)cc2C1] | 176 |
| 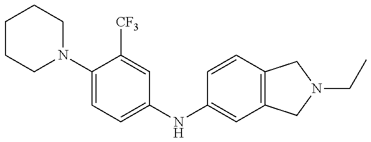 [SMILES: CCN1Cc2ccc(Nc3ccc(N4CCCCC4)c(C(F)(F)F)c3)cc2C1] | 177 |
| 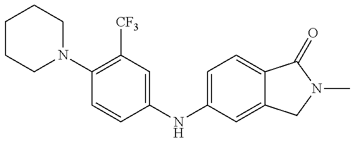 [SMILES: CN1Cc2cc(Nc3ccc(N4CCCCC4)c(C(F)(F)F)c3)ccc2C1=O] | 178 |
| 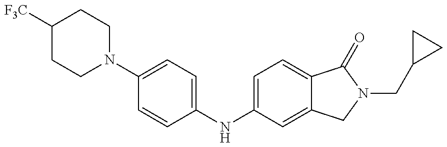 [SMILES: O=C1c2ccc(Nc3ccc(N4CCC(C(F)(F)F)CC4)cc3)cc2CN1CC1CC1] | 179 |
| 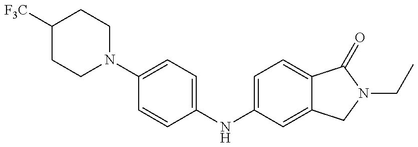 [SMILES: CCN1Cc2cc(Nc3ccc(N4CCC(C(F)(F)F)CC4)cc3)ccc2C1=O] | 180 |
| 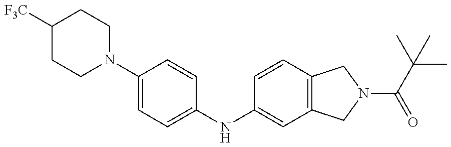 [SMILES: CC(C)(C)C(=O)N1Cc2ccc(Nc3ccc(N4CCC(C(F)(F)F)CC4)cc3)cc2C1] | 181 |
| 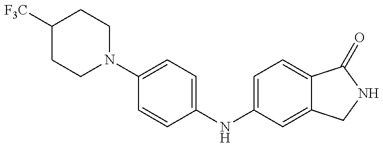 [SMILES: O=C1NCc2cc(Nc3ccc(N4CCC(C(F)(F)F)CC4)cc3)ccc21] | 182 |
| 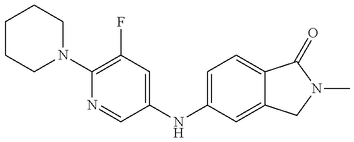 [SMILES: CN1Cc2cc(Nc3cnc(N4CCCCC4)c(F)c3)ccc2C1=O] | 183 |
| 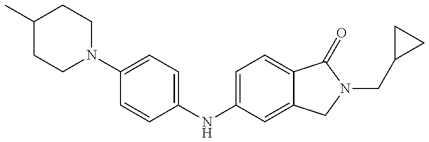 [SMILES: CC1CCN(c2ccc(Nc3ccc4c(c3)CN(CC3CC3)C4=O)cc2)CC1] | 184 |
| 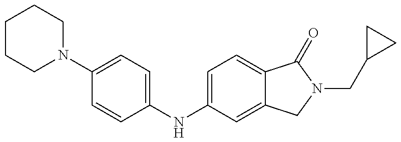 [SMILES: O=C1c2ccc(Nc3ccc(N4CCCCC4)cc3)cc2CN1CC1CC1] | 185 |
| 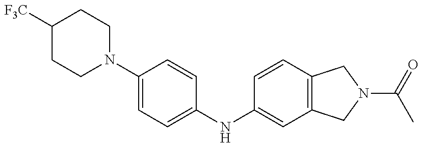 [SMILES: CC(=O)N1Cc2ccc(Nc3ccc(N4CCC(C(F)(F)F)CC4)cc3)cc2C1] | 186 |
| 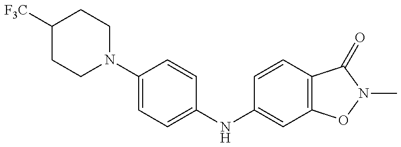 [SMILES: Cn1oc2cc(Nc3ccc(N4CCC(C(F)(F)F)CC4)cc3)ccc2c1=O] | 187 |
| 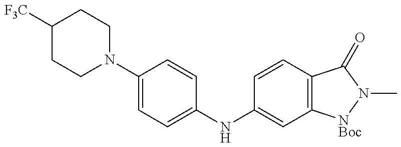 [SMILES: Cn1c(=O)c2ccc(Nc3ccc(N4CCC(C(F)(F)F)CC4)cc3)cc2n1C(=O)OC(C)(C)C] | 188 |
| 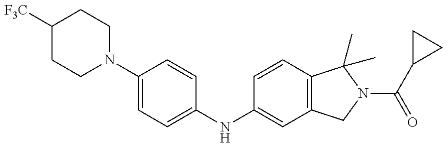 [SMILES: CC1(C)c2ccc(Nc3ccc(N4CCC(C(F)(F)F)CC4)cc3)cc2CN1C(=O)C1CC1] | 189 |
| 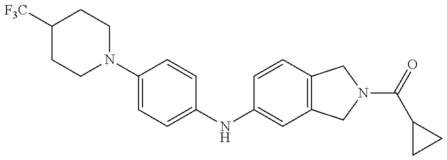 [SMILES: O=C(C1CC1)N1Cc2ccc(Nc3ccc(N4CCC(C(F)(F)F)CC4)cc3)cc2C1] | 190 |
| 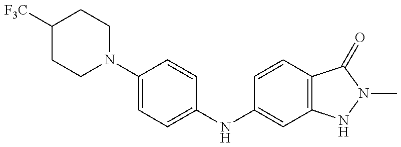 [SMILES: Cn1[nH]c2cc(Nc3ccc(N4CCC(C(F)(F)F)CC4)cc3)ccc2c1=O] | 191 |
| 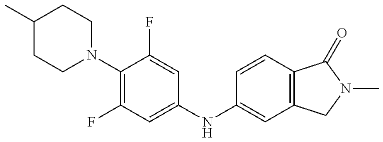 | 192 |
| 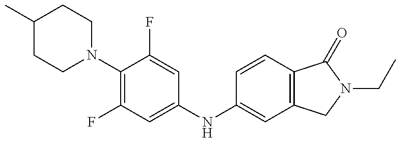 [SMILES: CCN1Cc2cc(Nc3cc(F)c(N4CCC(C)CC4)c(F)c3)ccc2C1=O] | 193 |
| 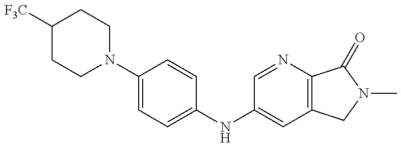 [SMILES: CN1Cc2cc(Nc3ccc(N4CCC(C(F)(F)F)CC4)cc3)cnc2C1=O] | 194 |
| 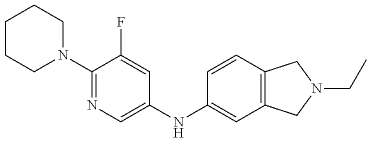 [SMILES: CCN1Cc2ccc(Nc3cnc(N4CCCCC4)c(F)c3)cc2C1] | 195 |
| 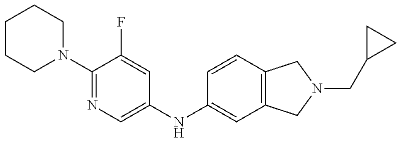 [SMILES: Fc1cc(Nc2ccc3c(c2)CN(CC2CC2)C3)cnc1N1CCCCC1] | 196 |
| 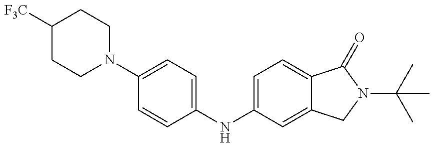 [SMILES: CC(C)(C)N1Cc2cc(Nc3ccc(N4CCC(C(F)(F)F)CC4)cc3)ccc2C1=O] | 197 |
| 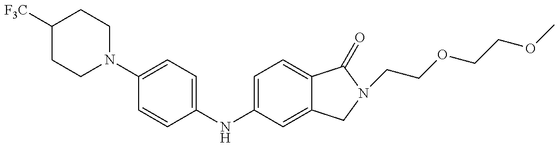 [SMILES: COCCOCCN1Cc2cc(Nc3ccc(N4CCC(C(F)(F)F)CC4)cc3)ccc2C1=O] | 198 |
| 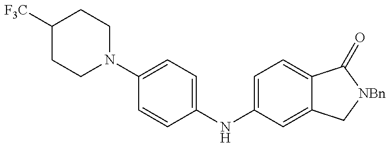 [SMILES: O=C1c2ccc(Nc3ccc(N4CCC(C(F)(F)F)CC4)cc3)cc2CN1Cc1ccccc1] | 199 |
| 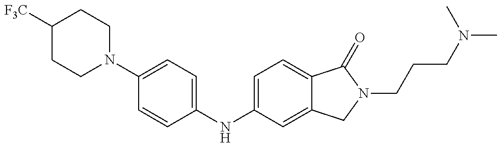 [SMILES: CN(C)CCCN1Cc2cc(Nc3ccc(N4CCC(C(F)(F)F)CC4)cc3)ccc2C1=O] | 200 |
| 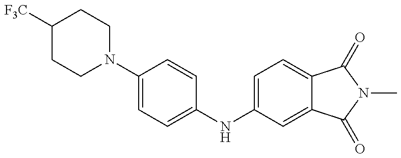 [SMILES: CN1C(=O)c2ccc(Nc3ccc(N4CCC(C(F)(F)F)CC4)cc3)cc2C1=O] | 201 |
| 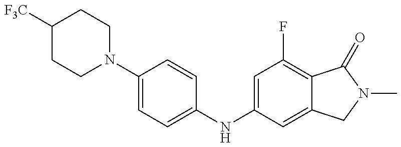 [SMILES: CN1Cc2cc(Nc3ccc(N4CCC(C(F)(F)F)CC4)cc3)cc(F)c2C1=O] | 202 |
| 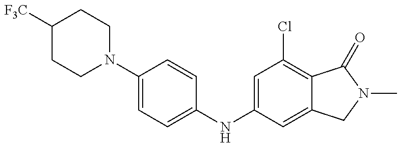 [SMILES: CN1Cc2cc(Nc3ccc(N4CCC(C(F)(F)F)CC4)cc3)cc(Cl)c2C1=O] | 203 |
| 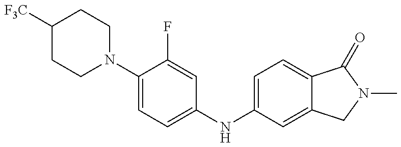 [SMILES: CN1Cc2cc(Nc3ccc(N4CCC(C(F)(F)F)CC4)c(F)c3)ccc2C1=O] | 204 |
| 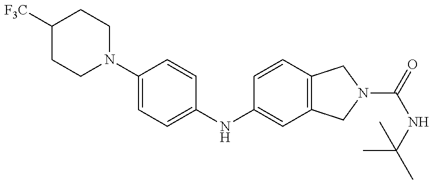 [SMILES: CC(C)(C)NC(=O)N1Cc2ccc(Nc3ccc(N4CCC(C(F)(F)F)CC4)cc3)cc2C1] | 205 |
| 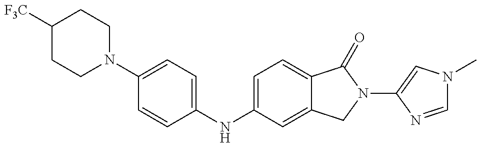 [SMILES: Cn1cnc(N2Cc3cc(Nc4ccc(N5CCC(C(F)(F)F)CC5)cc4)ccc3C2=O)c1] | 206 |
| 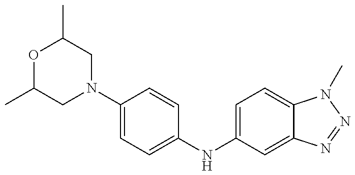 [SMILES: CC1CN(c2ccc(Nc3ccc4c(c3)nnn4C)cc2)CC(C)O1] | 207 |
| 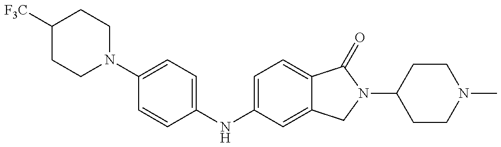 [SMILES: CN1CCC(N2Cc3cc(Nc4ccc(N5CCC(C(F)(F)F)CC5)cc4)ccc3C2=O)CC1] | 208 |
| 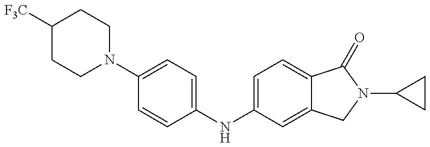 [SMILES: O=C1c2ccc(Nc3ccc(N4CCC(C(F)(F)F)CC4)cc3)cc2CN1C1CC1] | 209 |
| 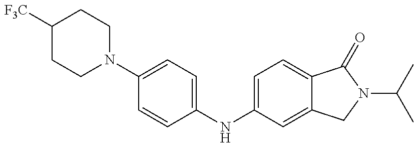 [SMILES: CC(C)N1Cc2cc(Nc3ccc(N4CCC(C(F)(F)F)CC4)cc3)ccc2C1=O] | 210 |
| 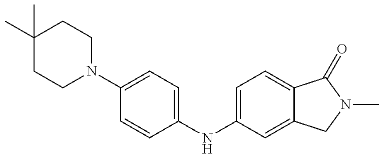 | 211 |
| 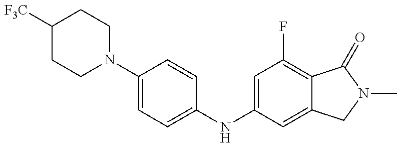 [SMILES: CN1Cc2cc(Nc3ccc(N4CCC(C(F)(F)F)CC4)cc3)cc(F)c2C1=O] | 212 |
| 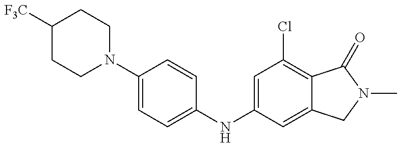 [SMILES: CN1Cc2cc(Nc3ccc(N4CCC(C(F)(F)F)CC4)cc3)cc(Cl)c2C1=O] | 213 |
| 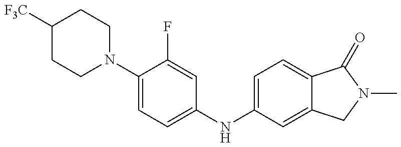 [SMILES: CN1Cc2cc(Nc3ccc(N4CCC(C(F)(F)F)CC4)c(F)c3)ccc2C1=O] | 214 |
| 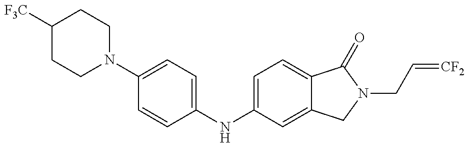 | 215 |
| 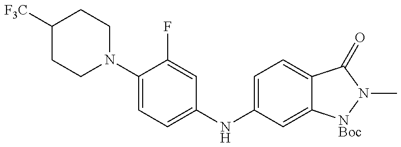 [SMILES: Cn1c(=O)c2ccc(Nc3ccc(N4CCC(C(F)(F)F)CC4)c(F)c3)cc2n1C(=O)OC(C)(C)C] | 216 |
| 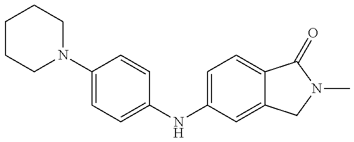 [SMILES: CN1Cc2cc(Nc3ccc(N4CCCCC4)cc3)ccc2C1=O] | 217 |
| 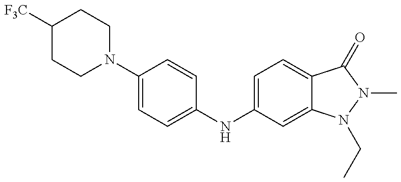 [SMILES: CCn1c2cc(Nc3ccc(N4CCC(C(F)(F)F)CC4)cc3)ccc2c(=O)n1C] | 218 |
| 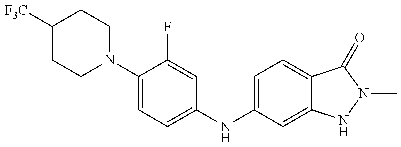 [SMILES: Cn1[nH]c2cc(Nc3ccc(N4CCC(C(F)(F)F)CC4)c(F)c3)ccc2c1=O] | 219 |
| 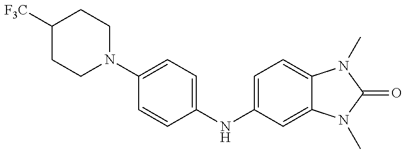 [SMILES: Cn1c(=O)n(C)c2cc(Nc3ccc(N4CCC(C(F)(F)F)CC4)cc3)ccc21] | 220 |
| 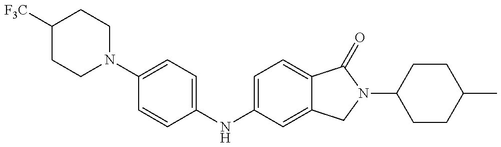 [SMILES: CC1CCC(N2Cc3cc(Nc4ccc(N5CCC(C(F)(F)F)CC5)cc4)ccc3C2=O)CC1] | 221 |
| 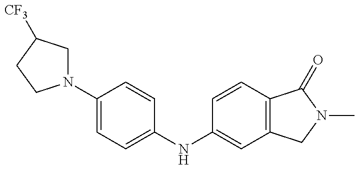 [SMILES: CN1Cc2cc(Nc3ccc(N4CCC(C(F)(F)F)C4)cc3)ccc2C1=O] | 222 |
| 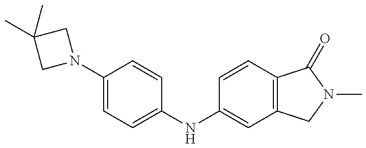 | 223 |
| 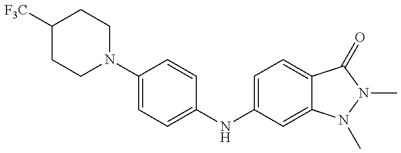 [SMILES: Cn1c(=O)c2ccc(Nc3ccc(N4CCC(C(F)(F)F)CC4)cc3)cc2n1C] | 224 |
| 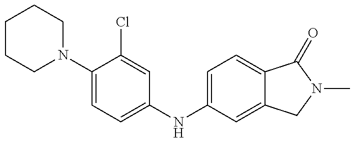 [SMILES: CN1Cc2cc(Nc3ccc(N4CCCCC4)c(Cl)c3)ccc2C1=O] | 225 |
| 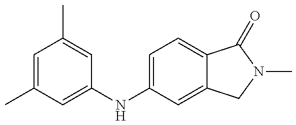 | 226 |
| 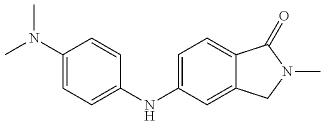 | 227 |
| 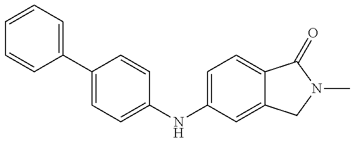 [SMILES: CN1Cc2cc(Nc3ccc(-c4ccccc4)cc3)ccc2C1=O] | 228 |
| 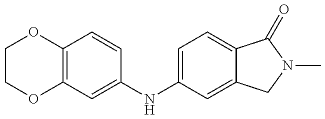 [SMILES: CN1Cc2cc(Nc3ccc4c(c3)OCCO4)ccc2C1=O] | 229 |
| 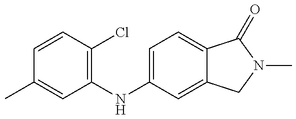 | 230 |
| 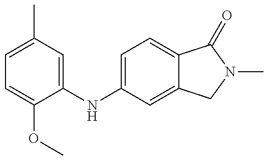 [SMILES: COc1ccc(C)cc1Nc1ccc2c(c1)CN(C)C2=O] | 231 |
| 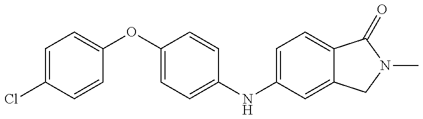 [SMILES: CN1Cc2cc(Nc3ccc(Oc4ccc(Cl)cc4)cc3)ccc2C1=O] | 232 |
| 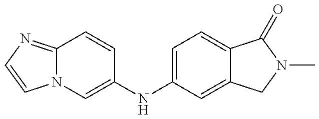 [SMILES: CN1Cc2cc(Nc3ccc4nccn4c3)ccc2C1=O] | 233 |
| 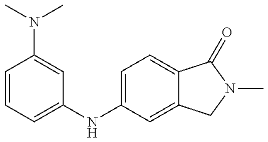 [SMILES: CN1Cc2cc(Nc3cccc(N(C)C)c3)ccc2C1=O] | 234 |
| 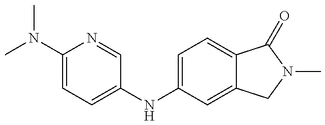 | 235 |
| 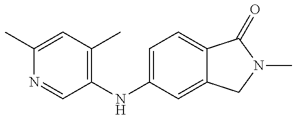 | 236 |
| 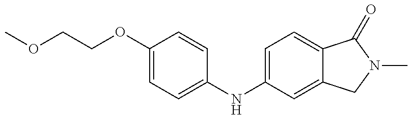 | 237 |
| 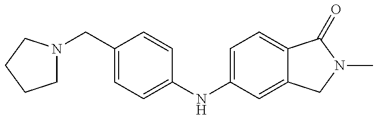 [SMILES: CN1Cc2cc(Nc3ccc(CN4CCCC4)cc3)ccc2C1=O] | 238 |
| 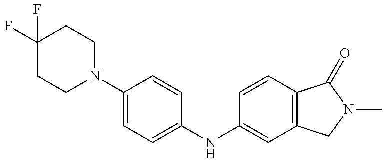 [SMILES: CN1Cc2cc(Nc3ccc(N4CCC(F)(F)CC4)cc3)ccc2C1=O] | 239 |
| 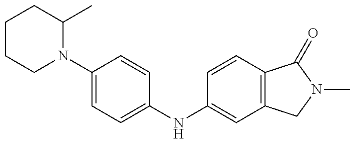 [SMILES: CC1CCCCN1c1ccc(Nc2ccc3c(c2)CN(C)C3=O)cc1] | 240 |
| 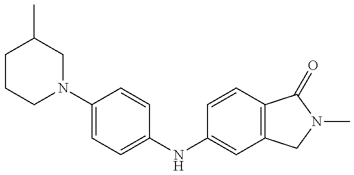 [SMILES: CC1CCCN(c2ccc(Nc3ccc4c(c3)CN(C)C4=O)cc2)C1] | 241 |
| 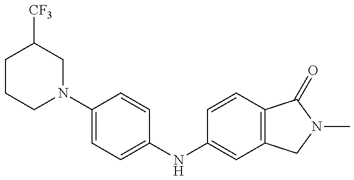 [SMILES: CN1Cc2cc(Nc3ccc(N4CCCC(C(F)(F)F)C4)cc3)ccc2C1=O] | 242 |
| 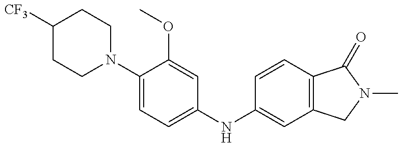 [SMILES: COc1cc(Nc2ccc3c(c2)CN(C)C3=O)ccc1N1CCC(C(F)(F)F)CC1] | 243 |
| 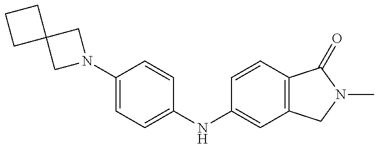 [SMILES: CN1Cc2cc(Nc3ccc(N4CC5(CCC5)C4)cc3)ccc2C1=O] | 244 |
| 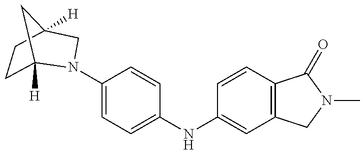 [SMILES: CN1Cc2cc(Nc3ccc(N4C[C@H]5CC[C@@H]4C5)cc3)ccc2C1=O] | 245 |
| 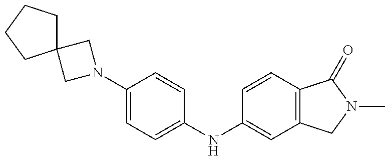 [SMILES: CN1Cc2cc(Nc3ccc(N4CC5(CCCC5)C4)cc3)ccc2C1=O] | 246 |
| 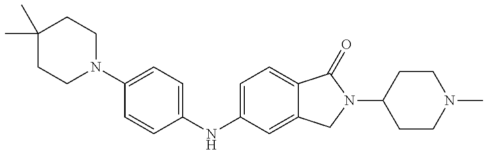 | 247 |
| 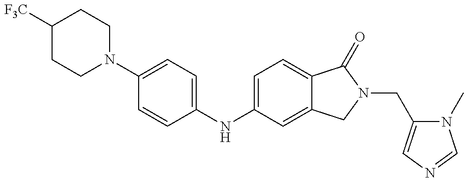 [SMILES: Cn1cncc1CN1Cc2cc(Nc3ccc(N4CCC(C(F)(F)F)CC4)cc3)ccc2C1=O] | 248 |
| 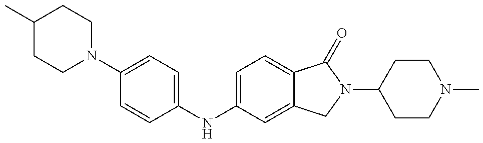 | 249 |
| 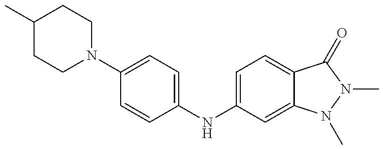 | 250 |
| 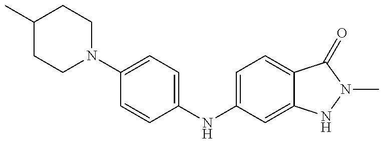 | 251 |
| 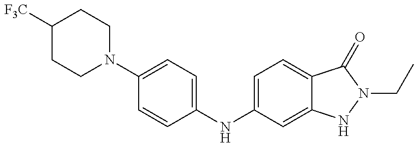 [SMILES: CCn1[nH]c2cc(Nc3ccc(N4CCC(C(F)(F)F)CC4)cc3)ccc2c1=O] | 252 |
| 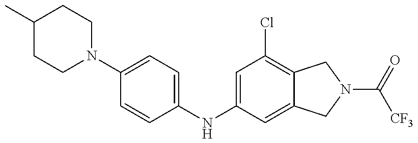 [SMILES: CC1CCN(c2ccc(Nc3cc(Cl)c4c(c3)CN(C(=O)C(F)(F)F)C4)cc2)CC1] | 253 |
| 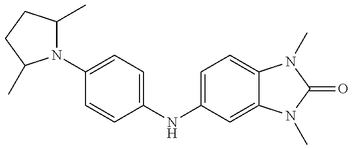 [SMILES: CC1CCC(C)N1c1ccc(Nc2ccc3c(c2)n(C)c(=O)n3C)cc1] | 254 |
| 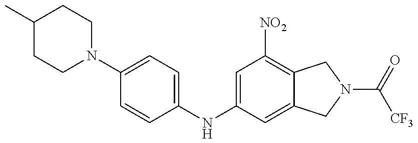 [SMILES: CC1CCN(c2ccc(Nc3cc4c(c([N+](=O)[O-])c3)CN(C(=O)C(F)(F)F)C4)cc2)CC1] | 255 |
| 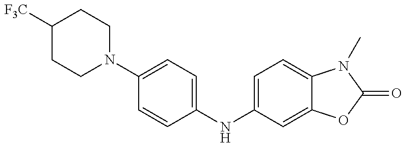 [SMILES: Cn1c(=O)oc2cc(Nc3ccc(N4CCC(C(F)(F)F)CC4)cc3)ccc21] | 256 |
| 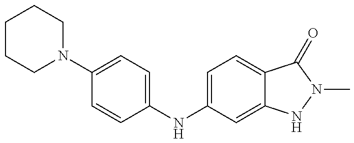 [SMILES: Cn1[nH]c2cc(Nc3ccc(N4CCCCC4)cc3)ccc2c1=O] | 257 |
| 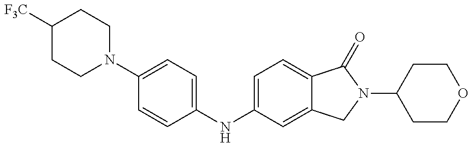 [SMILES: O=C1c2ccc(Nc3ccc(N4CCC(C(F)(F)F)CC4)cc3)cc2CN1C1CCOCC1] | 258 |
| 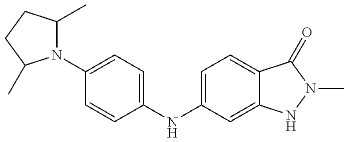 [SMILES: CC1CCC(C)N1c1ccc(Nc2ccc3c(=O)n(C)[nH]c3c2)cc1] | 259 |
| 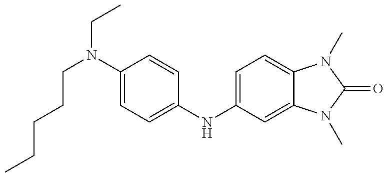 [SMILES: CCCCCN(CC)c1ccc(Nc2ccc3c(c2)n(C)c(=O)n3C)cc1] | 260 |
| 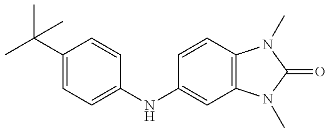 [SMILES: Cn1c(=O)n(C)c2cc(Nc3ccc(C(C)(C)C)cc3)ccc21] | 261 |
| 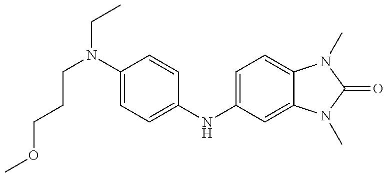 [SMILES: CCN(CCCOC)c1ccc(Nc2ccc3c(c2)n(C)c(=O)n3C)cc1] | 262 |
| 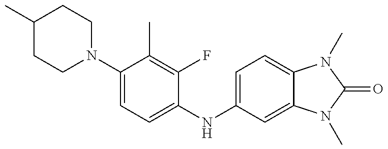 [SMILES: Cc1c(N2CCC(C)CC2)ccc(Nc2ccc3c(c2)n(C)c(=O)n3C)c1F] | 263 |
| 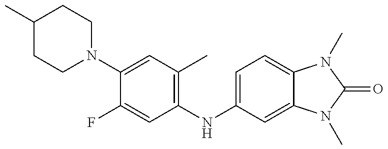 [SMILES: Cc1cc(N2CCC(C)CC2)c(F)cc1Nc1ccc2c(c1)n(C)c(=O)n2C] | 264 |
| 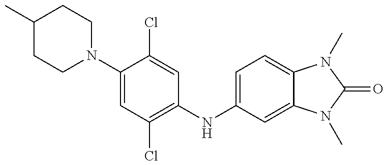 [SMILES: CC1CCN(c2cc(Cl)c(Nc3ccc4c(c3)n(C)c(=O)n4C)cc2Cl)CC1] | 265 |
| 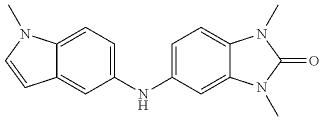 [SMILES: Cn1ccc2cc(Nc3ccc4c(c3)n(C)c(=O)n4C)ccc21] | 266 |
| 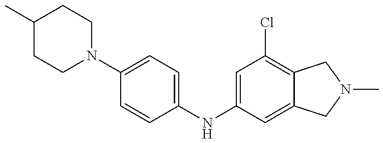 | 267 |
| 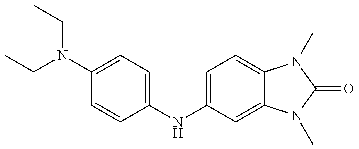 [SMILES: CCN(CC)c1ccc(Nc2ccc3c(c2)n(C)c(=O)n3C)cc1] | 268 |
| 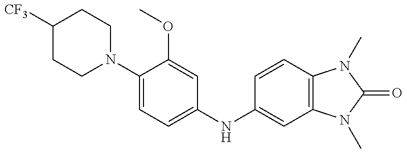 [SMILES: COc1cc(Nc2ccc3c(c2)n(C)c(=O)n3C)ccc1N1CCC(C(F)(F)F)CC1] | 269 |
| 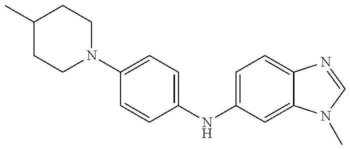 [SMILES: CC1CCN(c2ccc(Nc3ccc4ncn(C)c4c3)cc2)CC1] | 270 |
| 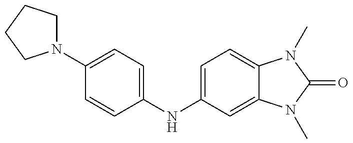 [SMILES: Cn1c(=O)n(C)c2cc(Nc3ccc(N4CCCC4)cc3)ccc21] | 271 |
| 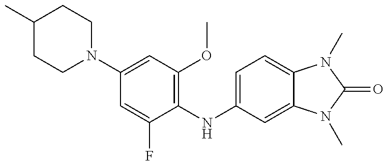 [SMILES: COc1cc(N2CCC(C)CC2)cc(F)c1Nc1ccc2c(c1)n(C)c(=O)n2C] | 272 |
| 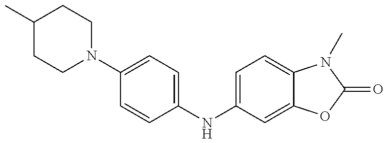 [SMILES: CC1CCN(c2ccc(Nc3ccc4c(c3)oc(=O)n4C)cc2)CC1] | 273 |
| 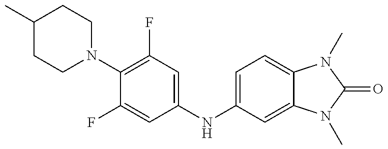 [SMILES: CC1CCN(c2c(F)cc(Nc3ccc4c(c3)n(C)c(=O)n4C)cc2F)CC1] | 274 |
| 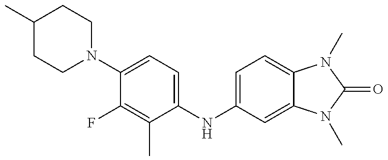 [SMILES: Cc1c(Nc2ccc3c(c2)n(C)c(=O)n3C)ccc(N2CCC(C)CC2)c1F] | 275 |
| 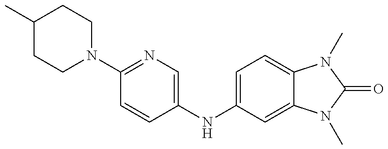 [SMILES: CC1CCN(c2ccc(Nc3ccc4c(c3)n(C)c(=O)n4C)cn2)CC1] | 276 |
| 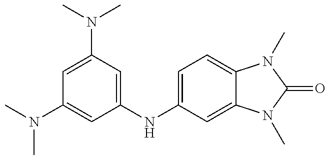 [SMILES: CN(C)c1cc(Nc2ccc3c(c2)n(C)c(=O)n3C)cc(N(C)C)c1] | 277 |
| 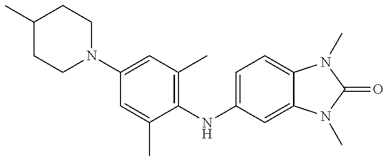 [SMILES: Cc1cc(N2CCC(C)CC2)cc(C)c1Nc1ccc2c(c1)n(C)c(=O)n2C] | 278 |
| 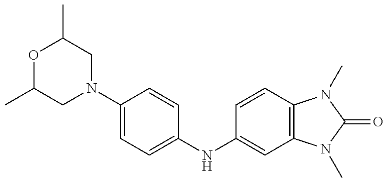 [SMILES: CC1CN(c2ccc(Nc3ccc4c(c3)n(C)c(=O)n4C)cc2)CC(C)O1] | 279 |
| 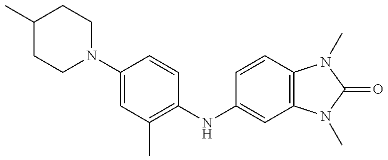 [SMILES: Cc1cc(N2CCC(C)CC2)ccc1Nc1ccc2c(c1)n(C)c(=O)n2C] | 280 |
| 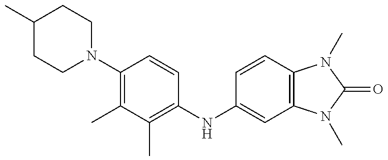 [SMILES: Cc1c(Nc2ccc3c(c2)n(C)c(=O)n3C)ccc(N2CCC(C)CC2)c1C] | 281 |
| 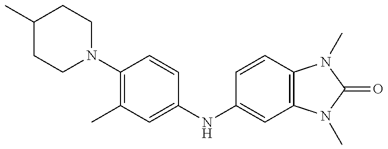 [SMILES: Cc1cc(Nc2ccc3c(c2)n(C)c(=O)n3C)ccc1N1CCC(C)CC1] | 282 |
| 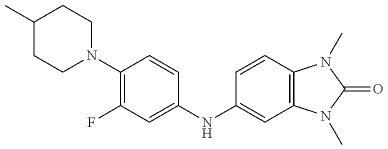 [SMILES: CC1CCN(c2ccc(Nc3ccc4c(c3)n(C)c(=O)n4C)cc2F)CC1] | 283 |
| 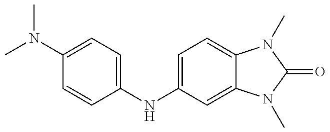 [SMILES: CN(C)c1ccc(Nc2ccc3c(c2)n(C)c(=O)n3C)cc1] | 284 |
| 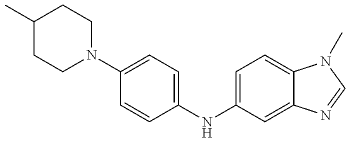 [SMILES: CC1CCN(c2ccc(Nc3ccc4c(c3)ncn4C)cc2)CC1] | 285 |
| 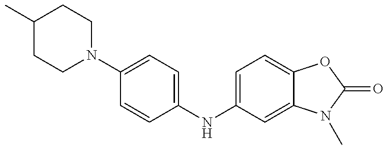 [SMILES: CC1CCN(c2ccc(Nc3ccc4oc(=O)n(C)c4c3)cc2)CC1] | 286 |
| 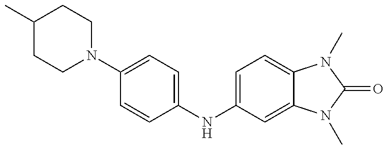 [SMILES: CC1CCN(c2ccc(Nc3ccc4c(c3)n(C)c(=O)n4C)cc2)CC1] | 287 |
| 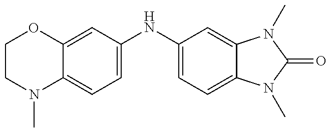 [SMILES: CN1CCOc2cc(Nc3ccc4c(c3)n(C)c(=O)n4C)ccc21] | 288 |
| 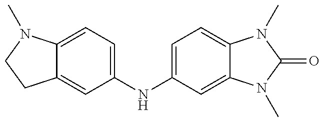 [SMILES: CN1CCc2cc(Nc3ccc4c(c3)n(C)c(=O)n4C)ccc21] | 289 |
| 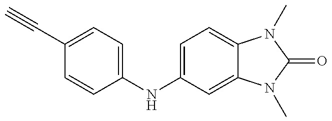 [SMILES: C#Cc1ccc(Nc2ccc3c(c2)n(C)c(=O)n3C)cc1] | 290 |
| 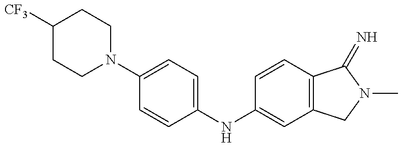 [SMILES: CN1Cc2cc(Nc3ccc(N4CCC(C(F)(F)F)CC4)cc3)ccc2C1=N] | 291 |
| 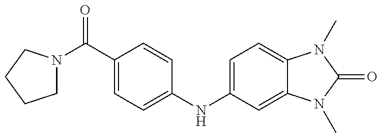 [SMILES: Cn1c(=O)n(C)c2cc(Nc3ccc(C(=O)N4CCCC4)cc3)ccc21] | 292 |
| 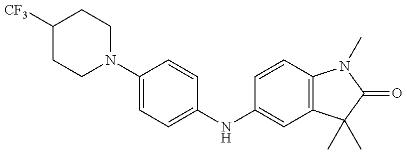 [SMILES: CN1C(=O)C(C)(C)c2cc(Nc3ccc(N4CCC(C(F)(F)F)CC4)cc3)ccc21] | 293 |
| 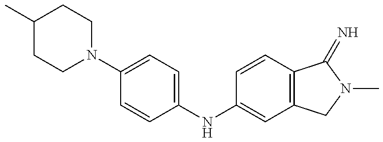 | 294 |
| 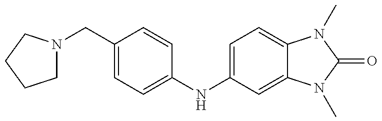 [SMILES: Cn1c(=O)n(C)c2cc(Nc3ccc(CN4CCCC4)cc3)ccc21] | 295 |
| 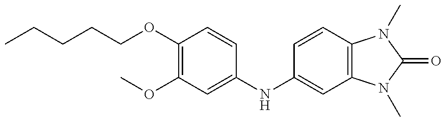 [SMILES: CCCCCOc1ccc(Nc2ccc3c(c2)n(C)c(=O)n3C)cc1OC] | 296 |
| 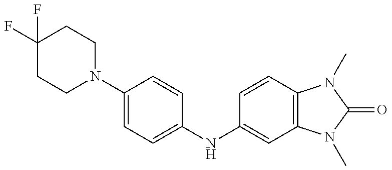 [SMILES: Cn1c(=O)n(C)c2cc(Nc3ccc(N4CCC(F)(F)CC4)cc3)ccc21] | 297 |
| 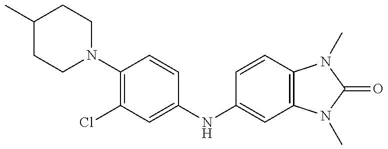 [SMILES: CC1CCN(c2ccc(Nc3ccc4c(c3)n(C)c(=O)n4C)cc2Cl)CC1] | 298 |
| 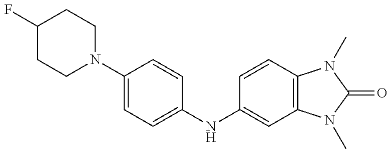 [SMILES: Cn1c(=O)n(C)c2cc(Nc3ccc(N4CCC(F)CC4)cc3)ccc21] | 299 |
| 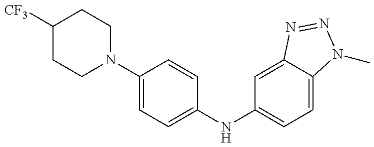 [SMILES: Cn1nnc2cc(Nc3ccc(N4CCC(C(F)(F)F)CC4)cc3)ccc21] | 300 |
| 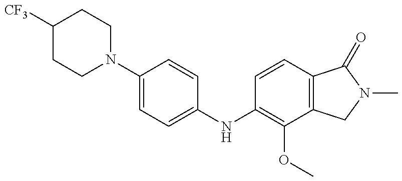 [SMILES: COc1c(Nc2ccc(N3CCC(C(F)(F)F)CC3)cc2)ccc2c1CN(C)C2=O] | 301 |
| 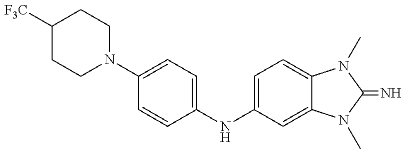 [SMILES: Cn1c(=N)n(C)c2cc(Nc3ccc(N4CCC(C(F)(F)F)CC4)cc3)ccc21] | 302 |
| 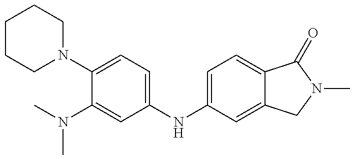 [SMILES: CN1Cc2cc(Nc3ccc(N4CCCCC4)c(N(C)C)c3)ccc2C1=O] | 303 |
| 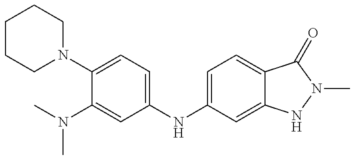 [SMILES: CN(C)c1cc(Nc2ccc3c(=O)n(C)[nH]c3c2)ccc1N1CCCCC1] | 304 |
| 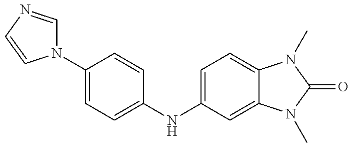 [SMILES: Cn1c(=O)n(C)c2cc(Nc3ccc(-n4ccnc4)cc3)ccc21] | 305 |
| 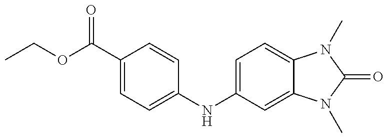 [SMILES: CCOC(=O)c1ccc(Nc2ccc3c(c2)n(C)c(=O)n3C)cc1] | 306 |
| 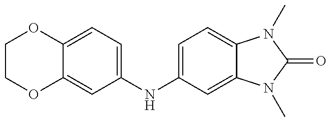 [SMILES: Cn1c(=O)n(C)c2cc(Nc3ccc4c(c3)OCCO4)ccc21] | 307 |
| 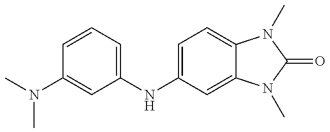 [SMILES: CN(C)c1cccc(Nc2ccc3c(c2)n(C)c(=O)n3C)c1] | 308 |
| 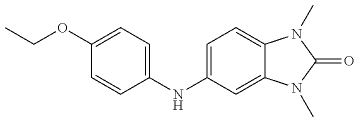 [SMILES: CCOc1ccc(Nc2ccc3c(c2)n(C)c(=O)n3C)cc1] | 309 |
| 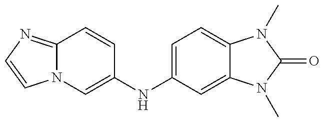 [SMILES: Cn1c(=O)n(C)c2cc(Nc3ccc4nccn4c3)ccc21] | 310 |
| 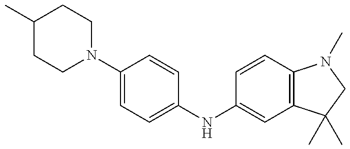 [SMILES: CC1CCN(c2ccc(Nc3ccc4c(c3)C(C)(C)CN4C)cc2)CC1] | 311 |
| 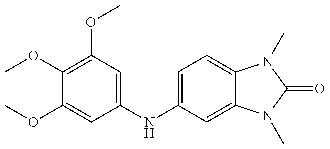 [SMILES: COc1cc(Nc2ccc3c(c2)n(C)c(=O)n3C)cc(OC)c1OC] | 312 |
| 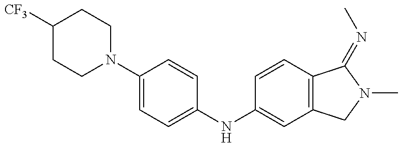 [SMILES: C/N=C1\c2ccc(Nc3ccc(N4CCC(C(F)(F)F)CC4)cc3)cc2CN1C] | 313 |
| 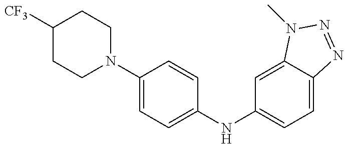 [SMILES: Cn1nnc2ccc(Nc3ccc(N4CCC(C(F)(F)F)CC4)cc3)cc21] | 314 |
| 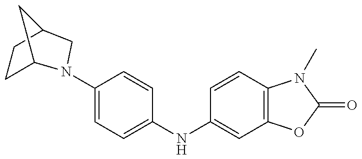 [SMILES: Cn1c(=O)oc2cc(Nc3ccc(N4CC5CCC4C5)cc3)ccc21] | 315 |
| 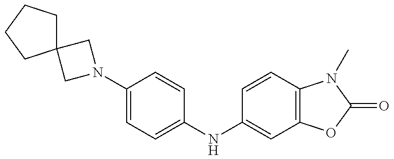 [SMILES: Cn1c(=O)oc2cc(Nc3ccc(N4CC5(CCCC5)C4)cc3)ccc21] | 316 |
| 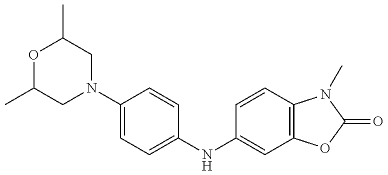 [SMILES: CC1CN(c2ccc(Nc3ccc4c(c3)oc(=O)n4C)cc2)CC(C)O1] | 317 |
| 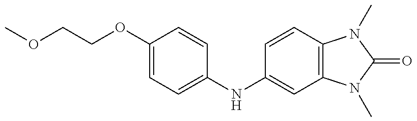 [SMILES: COCCOc1ccc(Nc2ccc3c(c2)n(C)c(=O)n3C)cc1] | 318 |
| 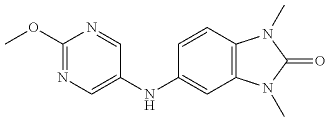 [SMILES: COc1ncc(Nc2ccc3c(c2)n(C)c(=O)n3C)cn1] | 319 |
| 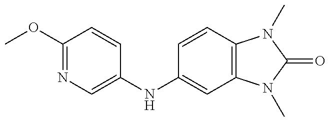 [SMILES: COc1ccc(Nc2ccc3c(c2)n(C)c(=O)n3C)cn1] | 320 |
| 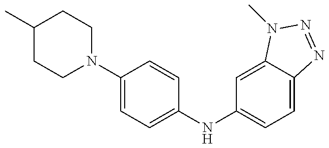 [SMILES: CC1CCN(c2ccc(Nc3ccc4nnn(C)c4c3)cc2)CC1] | 321 |
| 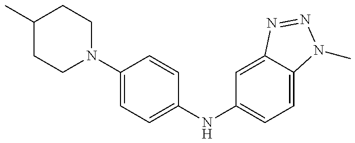 | 322 |
| 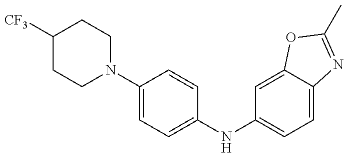 [SMILES: Cc1nc2ccc(Nc3ccc(N4CCC(C(F)(F)F)CC4)cc3)cc2o1] | 323 |
| 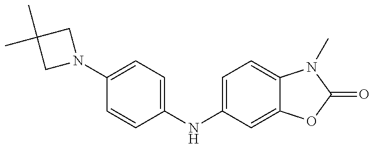 [SMILES: Cn1c(=O)oc2cc(Nc3ccc(N4CC(C)(C)C4)cc3)ccc21] | 324 |
| 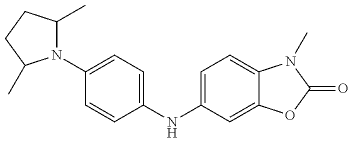 [SMILES: CC1CCC(C)N1c1ccc(Nc2ccc3c(c2)oc(=O)n3C)cc1] | 325 |
| 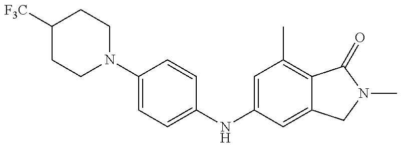 [SMILES: Cc1cc(Nc2ccc(N3CCC(C(F)(F)F)CC3)cc2)cc2c1C(=O)N(C)C2] | 326 |
| 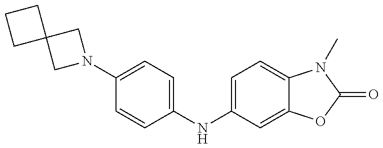 [SMILES: Cn1c(=O)oc2cc(Nc3ccc(N4CC5(CCC5)C4)cc3)ccc21] | 327 |
| 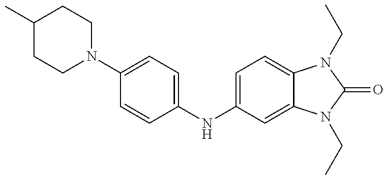 [SMILES: CCn1c(=O)n(CC)c2cc(Nc3ccc(N4CCC(C)CC4)cc3)ccc21] | 328 |
| 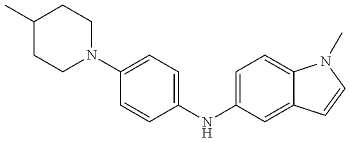 [SMILES: CC1CCN(c2ccc(Nc3ccc4c(ccn4C)c3)cc2)CC1] | 329 |
| 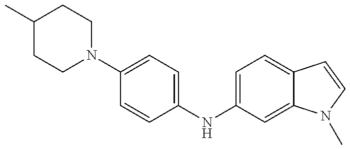 [SMILES: CC1CCN(c2ccc(Nc3ccc4ccn(C)c4c3)cc2)CC1] | 330 |
| 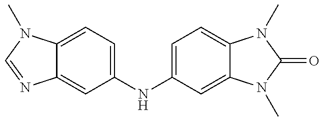 [SMILES: Cn1cnc2cc(Nc3ccc4c(c3)n(C)c(=O)n4C)ccc21] | 331 |
| 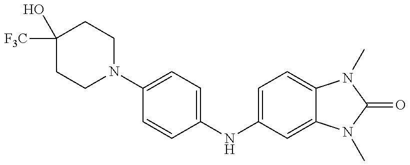 [SMILES: Cn1c(=O)n(C)c2cc(Nc3ccc(N4CCC(O)(C(F)(F)F)CC4)cc3)ccc21] | 332 |
| 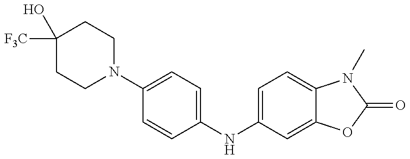 [SMILES: Cn1c(=O)oc2cc(Nc3ccc(N4CCC(O)(C(F)(F)F)CC4)cc3)ccc21] | 333 |
| 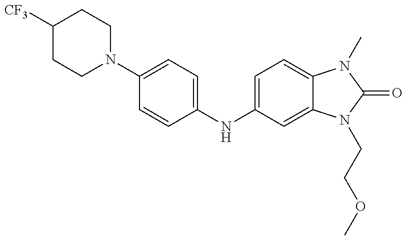 [SMILES: COCCn1c(=O)n(C)c2ccc(Nc3ccc(N4CCC(C(F)(F)F)CC4)cc3)cc21] | 334 |
| 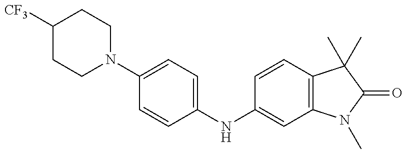 [SMILES: CN1C(=O)C(C)(C)c2ccc(Nc3ccc(N4CCC(C(F)(F)F)CC4)cc3)cc21] | 335 |
| 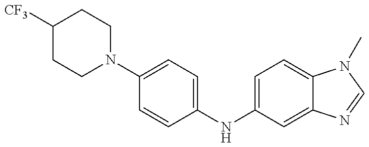 [SMILES: Cn1cnc2cc(Nc3ccc(N4CCC(C(F)(F)F)CC4)cc3)ccc21] | 336 |
| 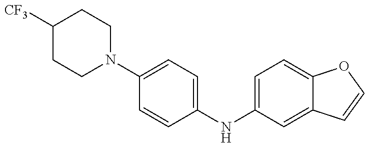 [SMILES: FC(F)(F)C1CCN(c2ccc(Nc3ccc4occc4c3)cc2)CC1] | 337 |
| 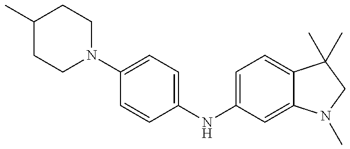 [SMILES: CC1CCN(c2ccc(Nc3ccc4c(c3)N(C)CC4(C)C)cc2)CC1] | 338 |
| 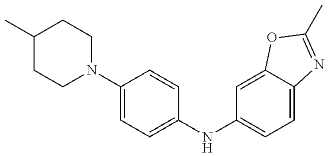 | 339 |
| 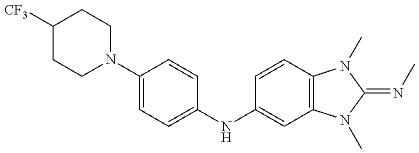 [SMILES: C/N=c1\n(C)c2ccc(Nc3ccc(N4CCC(C(F)(F)F)CC4)cc3)cc2n1C] | 340 |
| 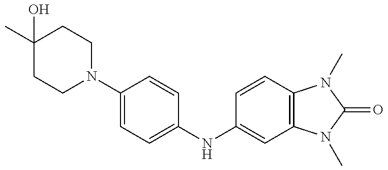 [SMILES: Cn1c(=O)n(C)c2cc(Nc3ccc(N4CCC(C)(O)CC4)cc3)ccc21] | 341 |
| 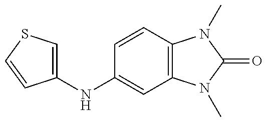 [SMILES: Cn1c(=O)n(C)c2cc(Nc3ccsc3)ccc21] | 342 |
| 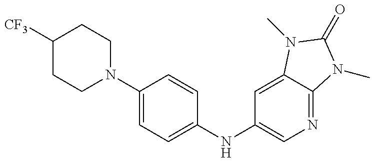 [SMILES: Cn1c(=O)n(C)c2ncc(Nc3ccc(N4CCC(C(F)(F)F)CC4)cc3)cc21] | 343 |
| 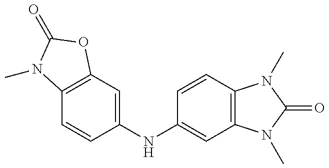 [SMILES: Cn1c(=O)oc2cc(Nc3ccc4c(c3)n(C)c(=O)n4C)ccc21] | 344 |
| 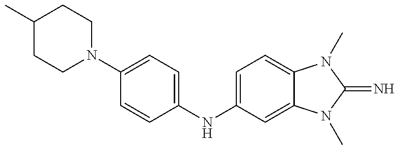 [SMILES: CC1CCN(c2ccc(Nc3ccc4c(c3)n(C)c(=N)n4C)cc2)CC1] | 345 |
| 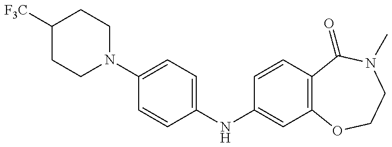 [SMILES: CN1CCOc2cc(Nc3ccc(N4CCC(C(F)(F)F)CC4)cc3)ccc2C1=O] | 346 |
| 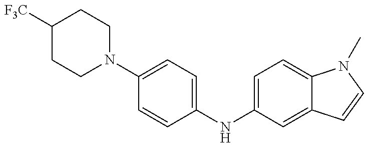 [SMILES: Cn1ccc2cc(Nc3ccc(N4CCC(C(F)(F)F)CC4)cc3)ccc21] | 347 |
| 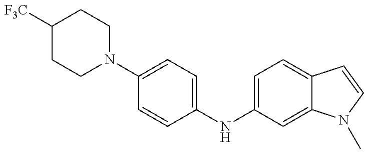 [SMILES: Cn1ccc2ccc(Nc3ccc(N4CCC(C(F)(F)F)CC4)cc3)cc21] | 348 |
| 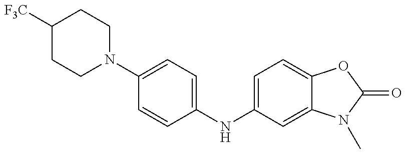 [SMILES: Cn1c(=O)oc2ccc(Nc3ccc(N4CCC(C(F)(F)F)CC4)cc3)cc21] | 349 |
| 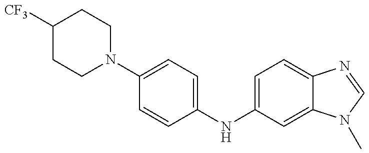 [SMILES: Cn1cnc2ccc(Nc3ccc(N4CCC(C(F)(F)F)CC4)cc3)cc21] | 350 |
| 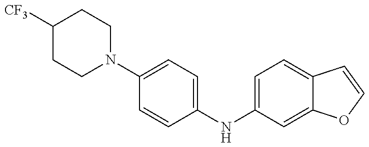 [SMILES: FC(F)(F)C1CCN(c2ccc(Nc3ccc4ccoc4c3)cc2)CC1] | 351 |
| 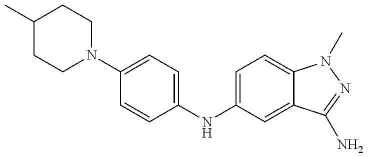 [SMILES: CC1CCN(c2ccc(Nc3ccc4c(c3)c(N)nn4C)cc2)CC1] | 352 |
| 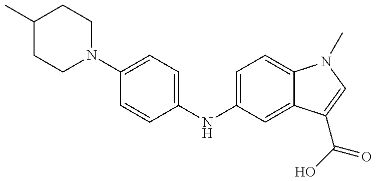 [SMILES: CC1CCN(c2ccc(Nc3ccc4c(c3)c(C(=O)O)cn4C)cc2)CC1] | 353 |
| 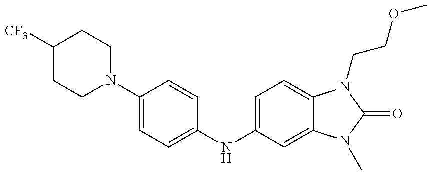 [SMILES: COCCn1c(=O)n(C)c2cc(Nc3ccc(N4CCC(C(F)(F)F)CC4)cc3)ccc21] | 354 |
| 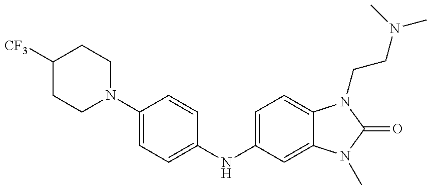 [SMILES: CN(C)CCn1c(=O)n(C)c2cc(Nc3ccc(N4CCC(C(F)(F)F)CC4)cc3)ccc21] | 355 |
| 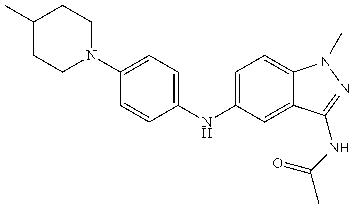 [SMILES: CC(=O)Nc1nn(C)c2ccc(Nc3ccc(N4CCC(C)CC4)cc3)cc12] | 356 |
| 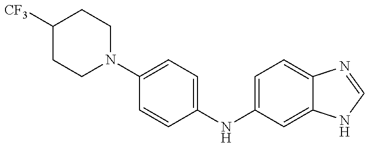 [SMILES: FC(F)(F)C1CCN(c2ccc(Nc3ccc4nc[nH]c4c3)cc2)CC1] | 357 |
| 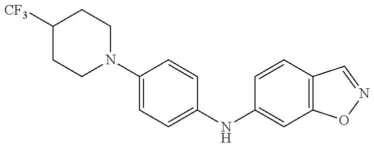 [SMILES: FC(F)(F)C1CCN(c2ccc(Nc3ccc4cnoc4c3)cc2)CC1] | 358 |
| 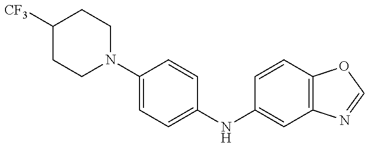 [SMILES: FC(F)(F)C1CCN(c2ccc(Nc3ccc4ocnc4c3)cc2)CC1] | 359 |
| 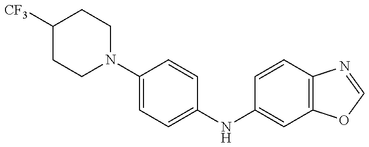 [SMILES: FC(F)(F)C1CCN(c2ccc(Nc3ccc4ncoc4c3)cc2)CC1] | 360 |
| 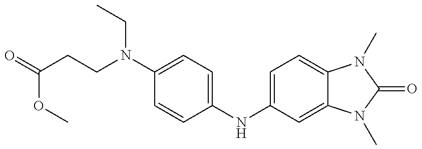 [SMILES: CCN(CCC(=O)OC)c1ccc(Nc2ccc3c(c2)n(C)c(=O)n3C)cc1] | 361 |
| 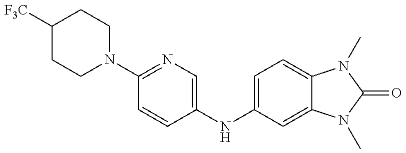 [SMILES: Cn1c(=O)n(C)c2cc(Nc3ccc(N4CCC(C(F)(F)F)CC4)nc3)ccc21] | 362 |
| 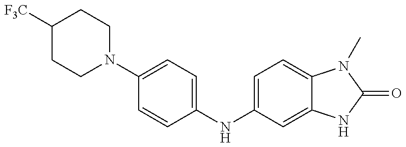 [SMILES: Cn1c(=O)[nH]c2cc(Nc3ccc(N4CCC(C(F)(F)F)CC4)cc3)ccc21] | 363 |
| 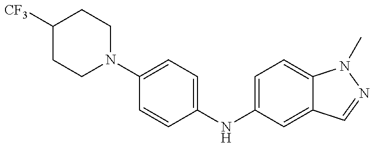 [SMILES: Cn1ncc2cc(Nc3ccc(N4CCC(C(F)(F)F)CC4)cc3)ccc21] | 364 |
| 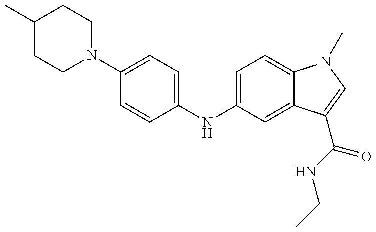 [SMILES: CCNC(=O)c1cn(C)c2ccc(Nc3ccc(N4CCC(C)CC4)cc3)cc12] | 365 |
| 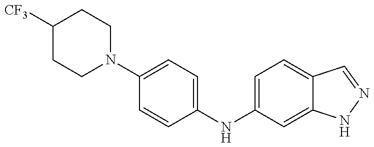 [SMILES: FC(F)(F)C1CCN(c2ccc(Nc3ccc4cn[nH]c4c3)cc2)CC1] | 366 |
| 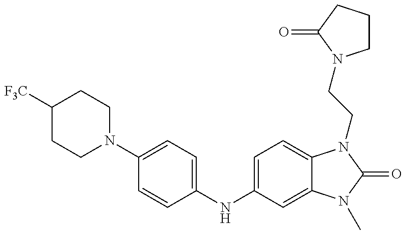 [SMILES: Cn1c(=O)n(CCN2CCCC2=O)c2ccc(Nc3ccc(N4CCC(C(F)(F)F)CC4)cc3)cc21] | 367 |
| 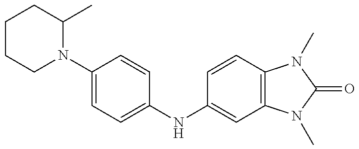 [SMILES: CC1CCCCN1c1ccc(Nc2ccc3c(c2)n(C)c(=O)n3C)cc1] | 368 |
| 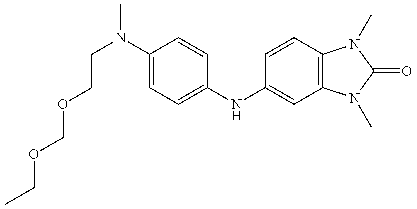 [SMILES: CCOCOCCN(C)c1ccc(Nc2ccc3c(c2)n(C)c(=O)n3C)cc1] | 369 |
| 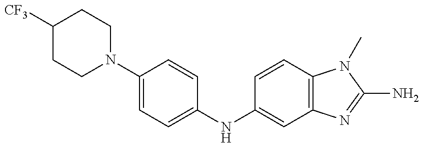 [SMILES: Cn1c(N)nc2cc(Nc3ccc(N4CCC(C(F)(F)F)CC4)cc3)ccc21] | 370 |
| 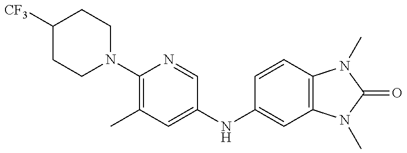 [SMILES: Cc1cc(Nc2ccc3c(c2)n(C)c(=O)n3C)cnc1N1CCC(C(F)(F)F)CC1] | 371 |
| 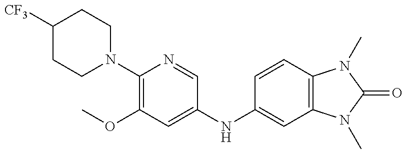 [SMILES: COc1cc(Nc2ccc3c(c2)n(C)c(=O)n3C)cnc1N1CCC(C(F)(F)F)CC1] | 372 |
| 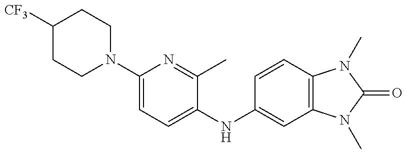 [SMILES: Cc1nc(N2CCC(C(F)(F)F)CC2)ccc1Nc1ccc2c(c1)n(C)c(=O)n2C] | 373 |
| 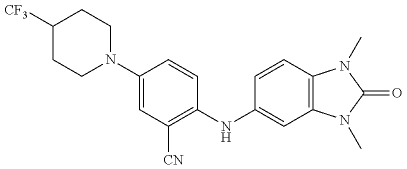 [SMILES: Cn1c(=O)n(C)c2cc(Nc3ccc(N4CCC(C(F)(F)F)CC4)cc3C#N)ccc21] | 374 |
| 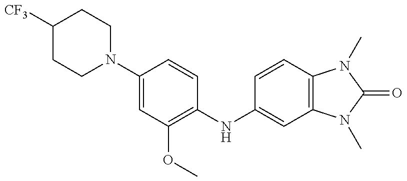 [SMILES: COc1cc(N2CCC(C(F)(F)F)CC2)ccc1Nc1ccc2c(c1)n(C)c(=O)n2C] | 375 |
| 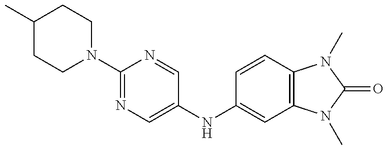 [SMILES: CC1CCN(c2ncc(Nc3ccc4c(c3)n(C)c(=O)n4C)cn2)CC1] | 376 |
| 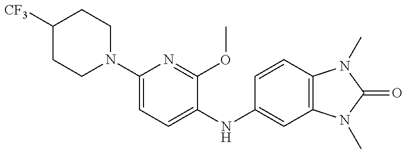 [SMILES: COc1nc(N2CCC(C(F)(F)F)CC2)ccc1Nc1ccc2c(c1)n(C)c(=O)n2C] | 377 |
| 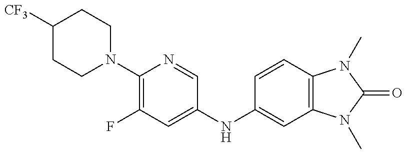 [SMILES: Cn1c(=O)n(C)c2cc(Nc3cnc(N4CCC(C(F)(F)F)CC4)c(F)c3)ccc21] | 378 |
| 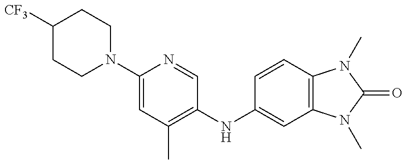 [SMILES: Cc1cc(N2CCC(C(F)(F)F)CC2)ncc1Nc1ccc2c(c1)n(C)c(=O)n2C] | 379 |
| 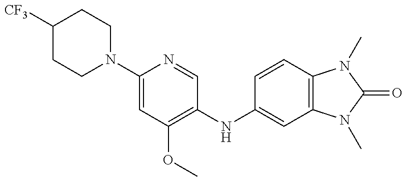 [SMILES: COc1cc(N2CCC(C(F)(F)F)CC2)ncc1Nc1ccc2c(c1)n(C)c(=O)n2C] | 380 |
| 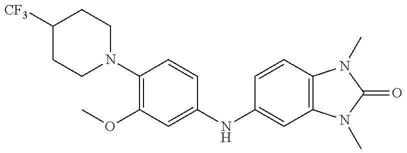 [SMILES: COc1cc(Nc2ccc3c(c2)n(C)c(=O)n3C)ccc1N1CCC(C(F)(F)F)CC1] | 381 |
| 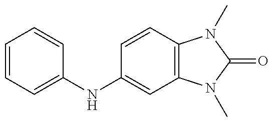 [SMILES: Cn1c(=O)n(C)c2cc(Nc3ccccc3)ccc21] | 382 |
| 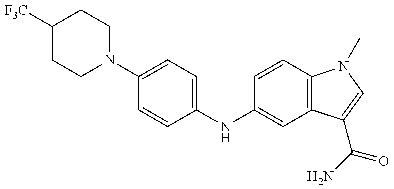 [SMILES: Cn1cc(C(N)=O)c2cc(Nc3ccc(N4CCC(C(F)(F)F)CC4)cc3)ccc21] | 383 |
| 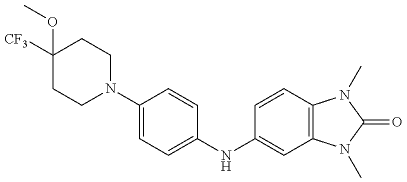 [SMILES: COC1(C(F)(F)F)CCN(c2ccc(Nc3ccc4c(c3)n(C)c(=O)n4C)cc2)CC1] | 384 |
| 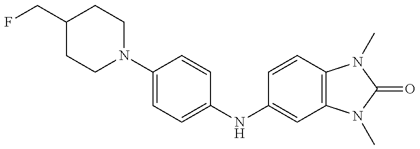 [SMILES: Cn1c(=O)n(C)c2cc(Nc3ccc(N4CCC(CF)CC4)cc3)ccc21] | 385 |
| 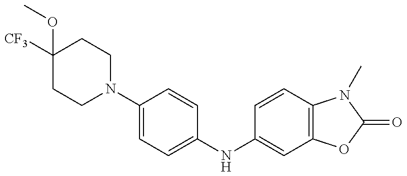 [SMILES: COC1(C(F)(F)F)CCN(c2ccc(Nc3ccc4c(c3)oc(=O)n4C)cc2)CC1] | 386 |
| 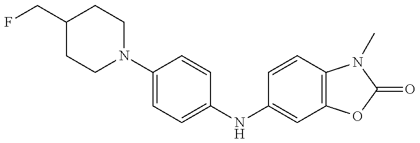 [SMILES: Cn1c(=O)oc2cc(Nc3ccc(N4CCC(CF)CC4)cc3)ccc21] | 387 |
| 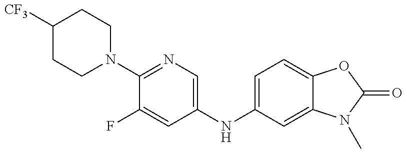 [SMILES: Cn1c(=O)oc2ccc(Nc3cnc(N4CCC(C(F)(F)F)CC4)c(F)c3)cc21] | 388 |
| 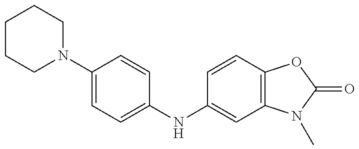 [SMILES: Cn1c(=O)oc2ccc(Nc3ccc(N4CCCCC4)cc3)cc21] | 389 |
| 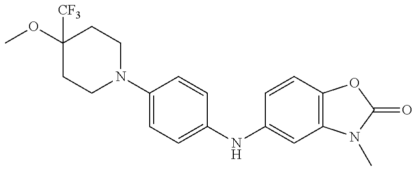 [SMILES: COC1(C(F)(F)F)CCN(c2ccc(Nc3ccc4oc(=O)n(C)c4c3)cc2)CC1] | 390 |
| 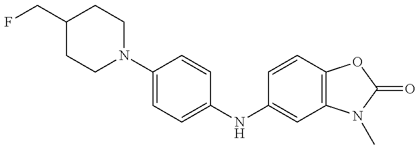 [SMILES: Cn1c(=O)oc2ccc(Nc3ccc(N4CCC(CF)CC4)cc3)cc21] | 391 |
| 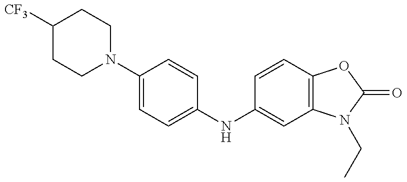 [SMILES: CCn1c(=O)oc2ccc(Nc3ccc(N4CCC(C(F)(F)F)CC4)cc3)cc21] | 392 |
| 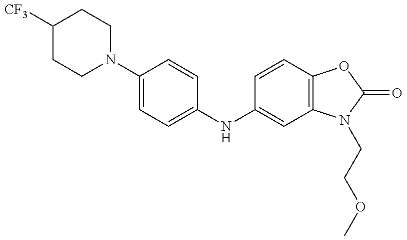 [SMILES: COCCn1c(=O)oc2ccc(Nc3ccc(N4CCC(C(F)(F)F)CC4)cc3)cc21] | 393 |
| 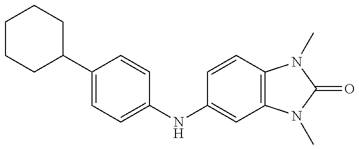 [SMILES: Cn1c(=O)n(C)c2cc(Nc3ccc(C4CCCCC4)cc3)ccc21] | 394 |
| 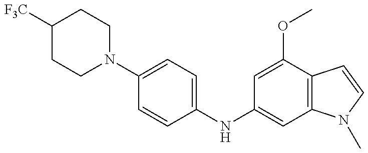 [SMILES: COc1cc(Nc2ccc(N3CCC(C(F)(F)F)CC3)cc2)cc2c1ccn2C] | 395 |
| 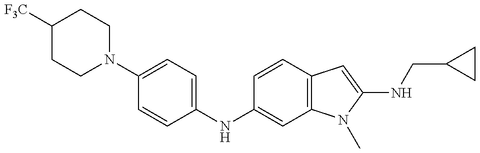 [SMILES: Cn1c(NCC2CC2)cc2ccc(Nc3ccc(N4CCC(C(F)(F)F)CC4)cc3)cc21] | 396 |
| 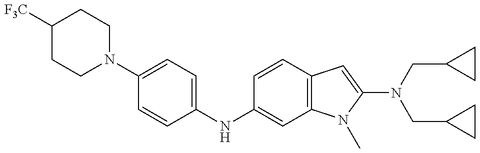 [SMILES: Cn1c(N(CC2CC2)CC2CC2)cc2ccc(Nc3ccc(N4CCC(C(F)(F)F)CC4)cc3)cc21] | 397 |
| 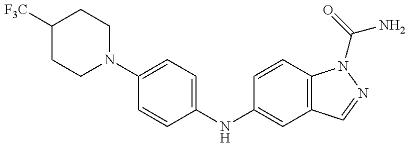 [SMILES: NC(=O)n1ncc2cc(Nc3ccc(N4CCC(C(F)(F)F)CC4)cc3)ccc21] | 398 |
| 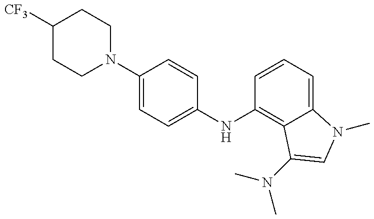 [SMILES: CN(C)c1cn(C)c2cccc(Nc3ccc(N4CCC(C(F)(F)F)CC4)cc3)c12] | 399 |
| 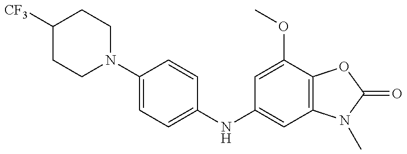 [SMILES: COc1cc(Nc2ccc(N3CCC(C(F)(F)F)CC3)cc2)cc2c1oc(=O)n2C] | 400 |
| 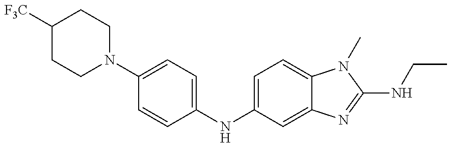 [SMILES: CCNc1nc2cc(Nc3ccc(N4CCC(C(F)(F)F)CC4)cc3)ccc2n1C] | 401 |
| 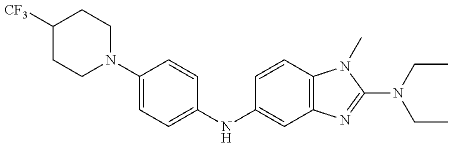 [SMILES: CCN(CC)c1nc2cc(Nc3ccc(N4CCC(C(F)(F)F)CC4)cc3)ccc2n1C] | 402 |
| 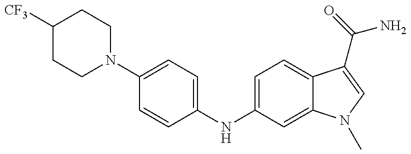 [SMILES: Cn1cc(C(N)=O)c2ccc(Nc3ccc(N4CCC(C(F)(F)F)CC4)cc3)cc21] | 403 |
| 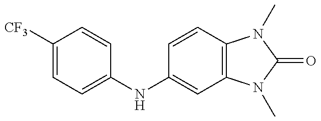 [SMILES: Cn1c(=O)n(C)c2cc(Nc3ccc(C(F)(F)F)cc3)ccc21] | 404 |
| 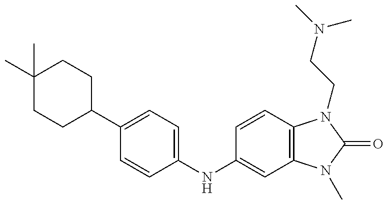 [SMILES: CN(C)CCn1c(=O)n(C)c2cc(Nc3ccc(C4CCC(C)(C)CC4)cc3)ccc21] | 405 |
| 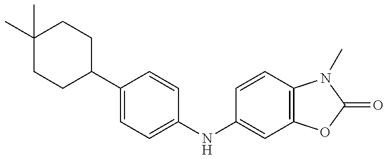 [SMILES: Cn1c(=O)oc2cc(Nc3ccc(C4CCC(C)(C)CC4)cc3)ccc21] | 406 |
| 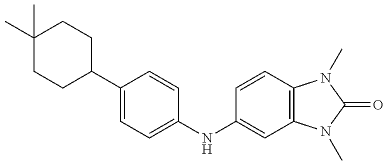 [SMILES: Cn1c(=O)n(C)c2cc(Nc3ccc(C4CCC(C)(C)CC4)cc3)ccc21] | 407 |
| 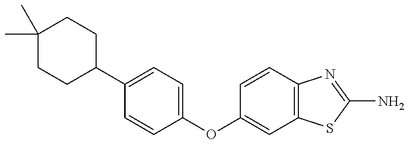 [SMILES: CC1(C)CCC(c2ccc(Oc3ccc4nc(N)sc4c3)cc2)CC1] | 408 |
| 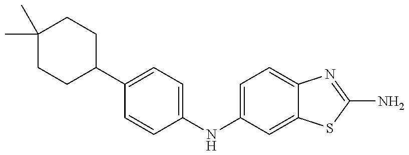 [SMILES: CC1(C)CCC(c2ccc(Nc3ccc4nc(N)sc4c3)cc2)CC1] | 409 |
| 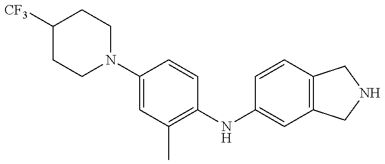 [SMILES: Cc1cc(N2CCC(C(F)(F)F)CC2)ccc1Nc1ccc2c(c1)CNC2] | 410 |
| 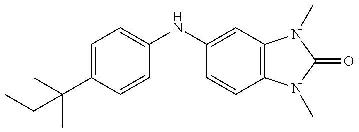 [SMILES: CCC(C)(C)c1ccc(Nc2ccc3c(c2)n(C)c(=O)n3C)cc1] | 411 |
| 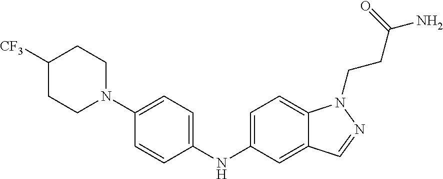 [SMILES: NC(=O)CCn1ncc2cc(Nc3ccc(N4CCC(C(F)(F)F)CC4)cc3)ccc21] | 412 |
| 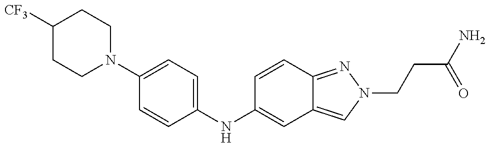 [SMILES: NC(=O)CCn1cc2cc(Nc3ccc(N4CCC(C(F)(F)F)CC4)cc3)ccc2n1] | 413 |
| 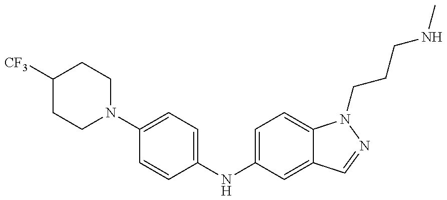 [SMILES: CNCCCn1ncc2cc(Nc3ccc(N4CCC(C(F)(F)F)CC4)cc3)ccc21] | 414 |
| 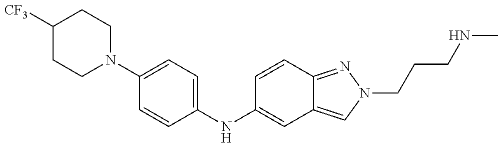 [SMILES: CNCCCn1cc2cc(Nc3ccc(N4CCC(C(F)(F)F)CC4)cc3)ccc2n1] | 415 |
| 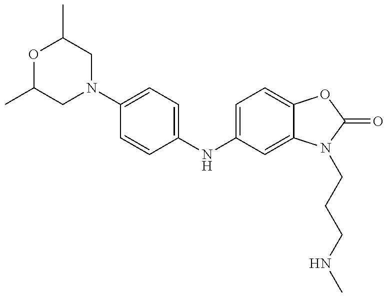 [SMILES: CNCCCn1c(=O)oc2ccc(Nc3ccc(N4CC(C)OC(C)C4)cc3)cc21] | 416 |
| 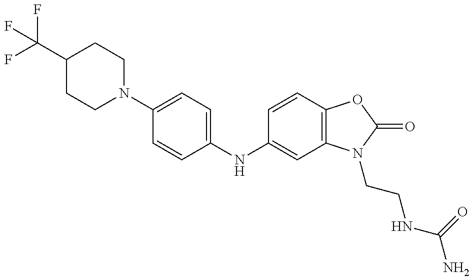 [SMILES: NC(=O)NCCn1c(=O)oc2ccc(Nc3ccc(N4CCC(C(F)(F)F)CC4)cc3)cc21] | 417 |
| 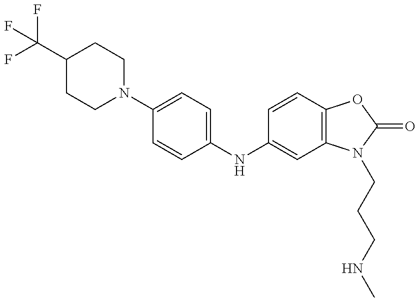 [SMILES: CNCCCn1c(=O)oc2ccc(Nc3ccc(N4CCC(C(F)(F)F)CC4)cc3)cc21] | 418 |
| 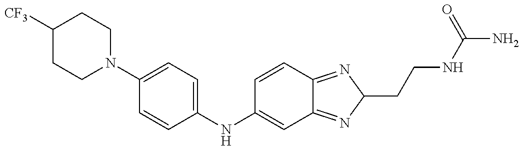 [SMILES: NC(=O)NCCC1N=c2ccc(Nc3ccc(N4CCC(C(F)(F)F)CC4)cc3)cc2=N1] | 419 |
| 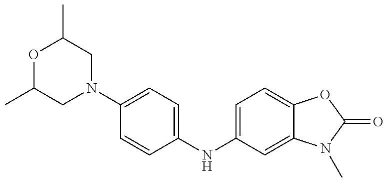 [SMILES: CC1CN(c2ccc(Nc3ccc4oc(=O)n(C)c4c3)cc2)CC(C)O1] | 420 |
| 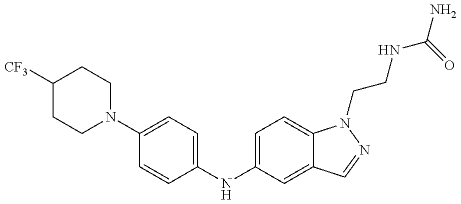 [SMILES: NC(=O)NCCn1ncc2cc(Nc3ccc(N4CCC(C(F)(F)F)CC4)cc3)ccc21] | 421 |
| 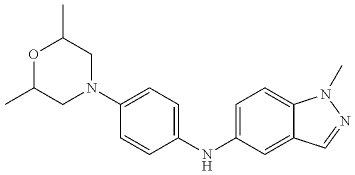 [SMILES: CC1CN(c2ccc(Nc3ccc4c(cnn4C)c3)cc2)CC(C)O1] | 422 |
| 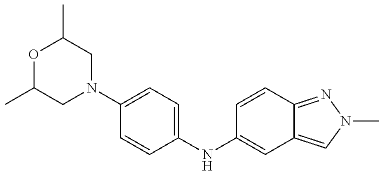 | 423 |
| 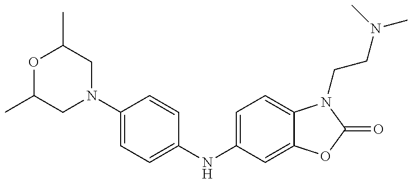 | 424 |
| 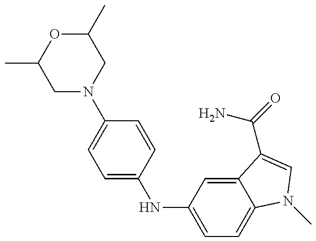 | 425 |
| 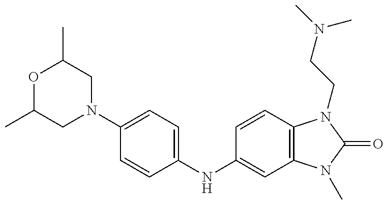 [SMILES: CC1CN(c2ccc(Nc3ccc4c(c3)n(C)c(=O)n4CCN(C)C)cc2)CC(C)O1] | 426 |
| 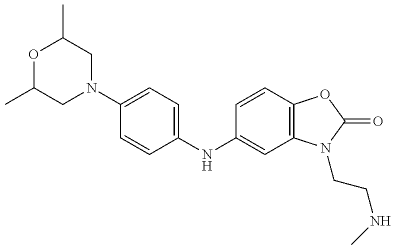 [SMILES: CNCCn1c(=O)oc2ccc(Nc3ccc(N4CC(C)OC(C)C4)cc3)cc21] | 427 |
| 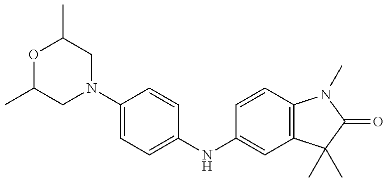 [SMILES: CC1CN(c2ccc(Nc3ccc4c(c3)C(C)(C)C(=O)N4C)cc2)CC(C)O1] | 428 |
| 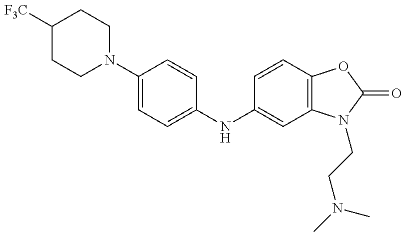 [SMILES: CN(C)CCn1c(=O)oc2ccc(Nc3ccc(N4CCC(C(F)(F)F)CC4)cc3)cc21] | 429 |
| 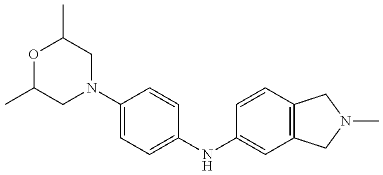 | 430 |
| 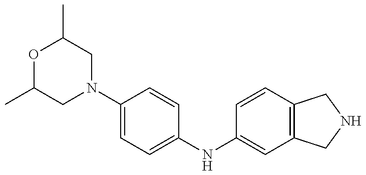 [SMILES: CC1CN(c2ccc(Nc3ccc4c(c3)CNC4)cc2)CC(C)O1] | 431 |
| 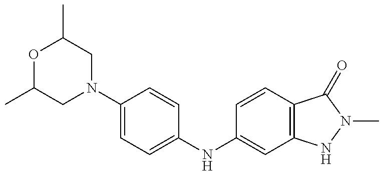 | 432 |
| 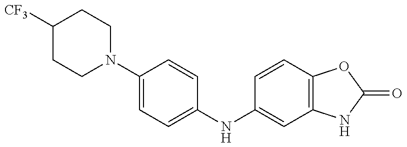 [SMILES: O=c1[nH]c2cc(Nc3ccc(N4CCC(C(F)(F)F)CC4)cc3)ccc2o1] | 433 |
| 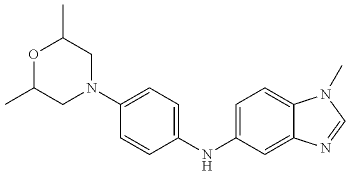 [SMILES: CC1CN(c2ccc(Nc3ccc4c(c3)ncn4C)cc2)CC(C)O1] | 434 |
| 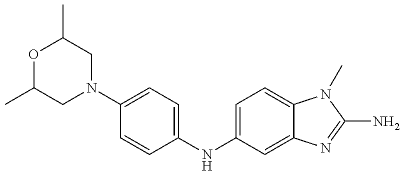 [SMILES: CC1CN(c2ccc(Nc3ccc4c(c3)nc(N)n4C)cc2)CC(C)O1] | 435 |
| 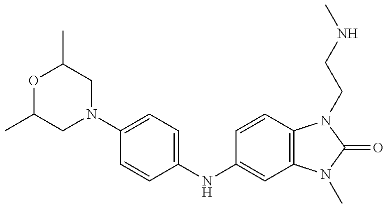 [SMILES: CNCCn1c(=O)n(C)c2cc(Nc3ccc(N4CC(C)OC(C)C4)cc3)ccc21] | 436 |
| 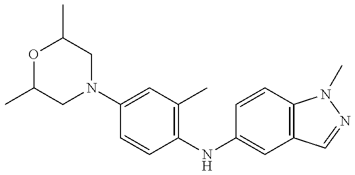 [SMILES: Cc1cc(N2CC(C)OC(C)C2)ccc1Nc1ccc2c(cnn2C)c1] | 437 |
| 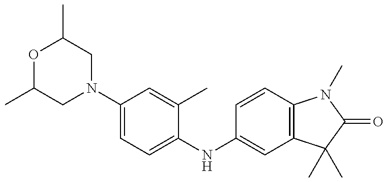 [SMILES: Cc1cc(N2CC(C)OC(C)C2)ccc1Nc1ccc2c(c1)C(C)(C)C(=O)N2C] | 438 |
| 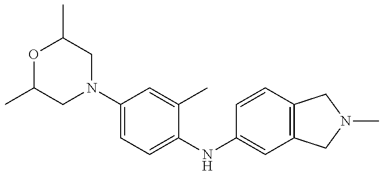 | 439 |
| 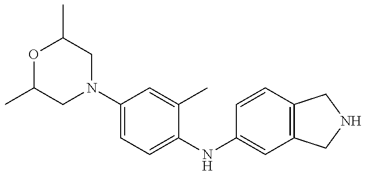 [SMILES: Cc1cc(N2CC(C)OC(C)C2)ccc1Nc1ccc2c(c1)CNC2] | 440 |
| 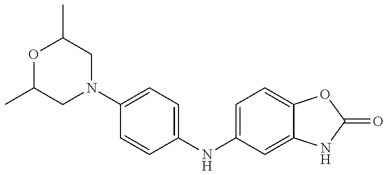 [SMILES: CC1CN(c2ccc(Nc3ccc4oc(=O)[nH]c4c3)cc2)CC(C)O1] | 441 |
| 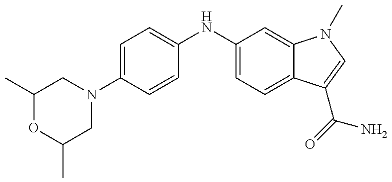 [SMILES: CC1CN(c2ccc(Nc3ccc4c(C(N)=O)cn(C)c4c3)cc2)CC(C)O1] | 442 |
| 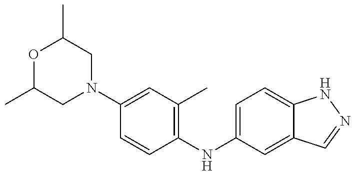 [SMILES: Cc1cc(N2CC(C)OC(C)C2)ccc1Nc1ccc2[nH]ncc2c1] | 443 |
| 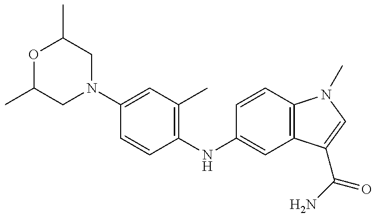 [SMILES: Cc1cc(N2CC(C)OC(C)C2)ccc1Nc1ccc2c(c1)c(C(N)=O)cn2C] | 444 |
| 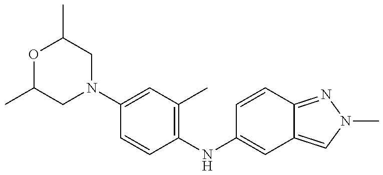 | 445 |
| 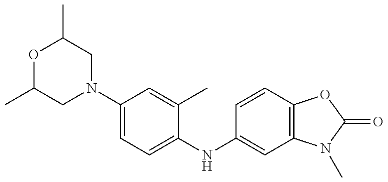 [SMILES: Cc1cc(N2CC(C)OC(C)C2)ccc1Nc1ccc2oc(=O)n(C)c2c1] | 446 |
| 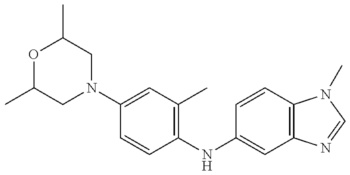 [SMILES: Cc1cc(N2CC(C)OC(C)C2)ccc1Nc1ccc2c(c1)ncn2C] | 447 |
| 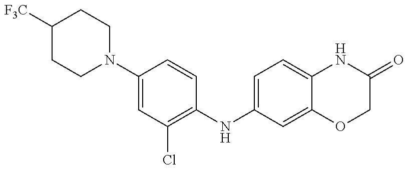 [SMILES: O=C1COc2cc(Nc3ccc(N4CCC(C(F)(F)F)CC4)cc3Cl)ccc2N1] | 448 |
| 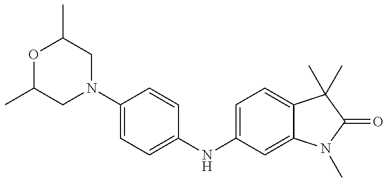 [SMILES: CC1CN(c2ccc(Nc3ccc4c(c3)N(C)C(=O)C4(C)C)cc2)CC(C)O1] | 449 |
| 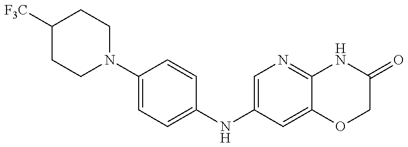 [SMILES: O=C1COc2cc(Nc3ccc(N4CCC(C(F)(F)F)CC4)cc3)cnc2N1] | 450 |
| 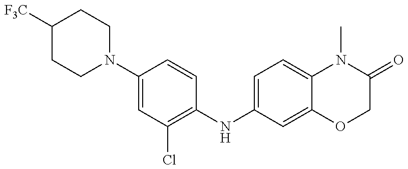 [SMILES: CN1C(=O)COc2cc(Nc3ccc(N4CCC(C(F)(F)F)CC4)cc3Cl)ccc21] | 451 |
| 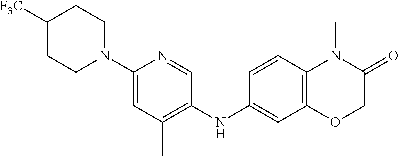 [SMILES: Cc1cc(N2CCC(C(F)(F)F)CC2)ncc1Nc1ccc2c(c1)OCC(=O)N2C] | 452 |
| 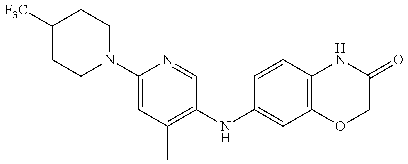 [SMILES: Cc1cc(N2CCC(C(F)(F)F)CC2)ncc1Nc1ccc2c(c1)OCC(=O)N2] | 453 |
| 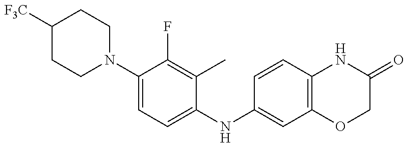 [SMILES: Cc1c(Nc2ccc3c(c2)OCC(=O)N3)ccc(N2CCC(C(F)(F)F)CC2)c1F] | 454 |
| 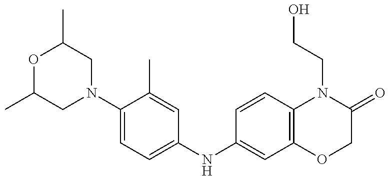 [SMILES: Cc1cc(Nc2ccc3c(c2)OCC(=O)N3CCO)ccc1N1CC(C)OC(C)C1] | 455 |
| 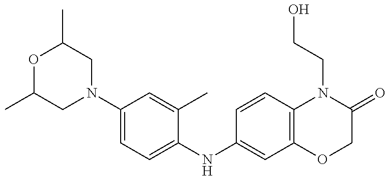 [SMILES: Cc1cc(N2CC(C)OC(C)C2)ccc1Nc1ccc2c(c1)OCC(=O)N2CCO] | 456 |
| 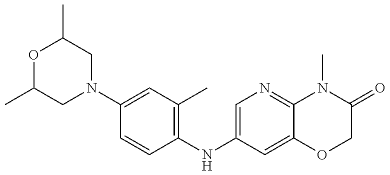 [SMILES: Cc1cc(N2CC(C)OC(C)C2)ccc1Nc1cnc2c(c1)OCC(=O)N2C] | 457 |
| 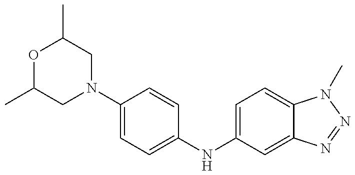 [SMILES: CC1CN(c2ccc(Nc3ccc4c(c3)nnn4C)cc2)CC(C)O1] | 458 |
| 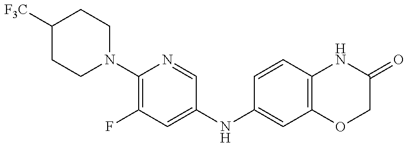 [SMILES: O=C1COc2cc(Nc3cnc(N4CCC(C(F)(F)F)CC4)c(F)c3)ccc2N1] | 459 |
| 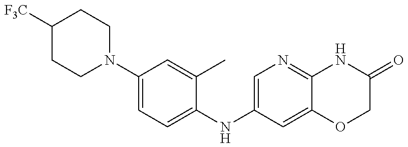 [SMILES: Cc1cc(N2CCC(C(F)(F)F)CC2)ccc1Nc1cnc2c(c1)OCC(=O)N2] | 460 |
| 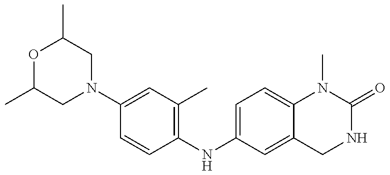 [SMILES: Cc1cc(N2CC(C)OC(C)C2)ccc1Nc1ccc2c(c1)CNC(=O)N2C] | 461 |
| 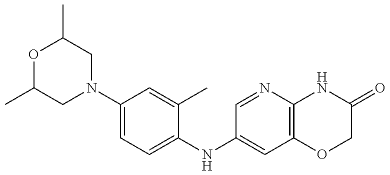 [SMILES: Cc1cc(N2CC(C)OC(C)C2)ccc1Nc1cnc2c(c1)OCC(=O)N2] | 462 |
| 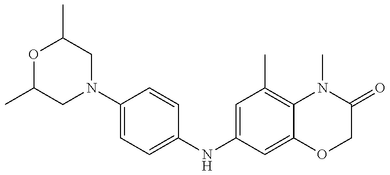 [SMILES: Cc1cc(Nc2ccc(N3CC(C)OC(C)C3)cc2)cc2c1N(C)C(=O)CO2] | 463 |
| 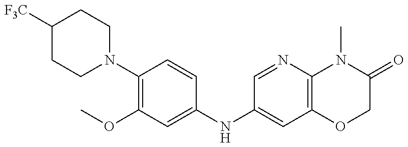 [SMILES: COc1cc(Nc2cnc3c(c2)OCC(=O)N3C)ccc1N1CCC(C(F)(F)F)CC1] | 464 |
| 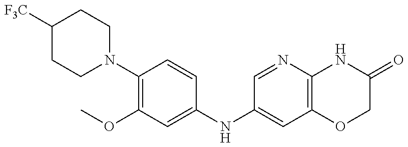 [SMILES: COc1cc(Nc2cnc3c(c2)OCC(=O)N3)ccc1N1CCC(C(F)(F)F)CC1] | 465 |
| 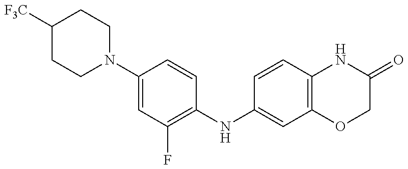 [SMILES: O=C1COc2cc(Nc3ccc(N4CCC(C(F)(F)F)CC4)cc3F)ccc2N1] | 466 |
| 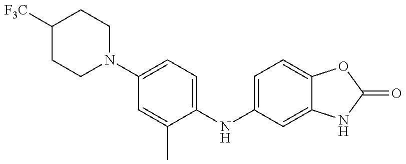 [SMILES: Cc1cc(N2CCC(C(F)(F)F)CC2)ccc1Nc1ccc2oc(=O)[nH]c2c1] | 467 |
| 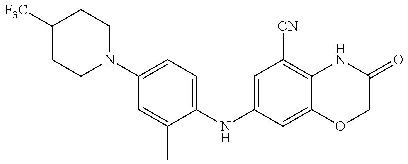 [SMILES: Cc1cc(N2CCC(C(F)(F)F)CC2)ccc1Nc1cc(C#N)c2c(c1)OCC(=O)N2] | 468 |
| 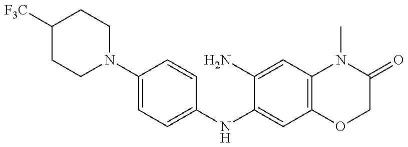 [SMILES: CN1C(=O)COc2cc(Nc3ccc(N4CCC(C(F)(F)F)CC4)cc3)c(N)cc21] | 469 |
| 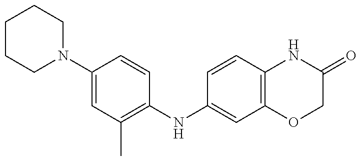 [SMILES: Cc1cc(N2CCCCC2)ccc1Nc1ccc2c(c1)OCC(=O)N2] | 470 |
| 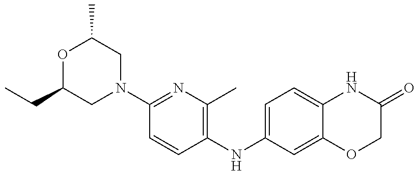 [SMILES: CC[C@@H]1CN(c2ccc(Nc3ccc4c(c3)OCC(=O)N4)c(C)n2)C[C@@H](C)O1] | 471 |
| 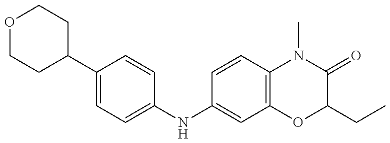 [SMILES: CCC1Oc2cc(Nc3ccc(C4CCOCC4)cc3)ccc2N(C)C1=O] | 472 |
| 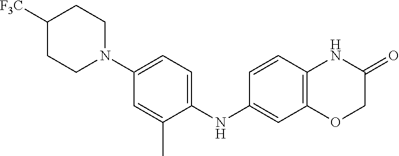 [SMILES: Cc1cc(N2CCC(C(F)(F)F)CC2)ccc1Nc1ccc2c(c1)OCC(=O)N2] | 473 |
| 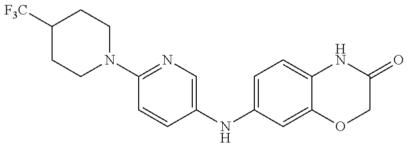 [SMILES: O=C1COc2cc(Nc3ccc(N4CCC(C(F)(F)F)CC4)nc3)ccc2N1] | 474 |
| 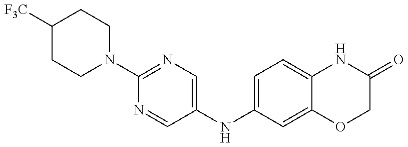 [SMILES: O=C1COc2cc(Nc3cnc(N4CCC(C(F)(F)F)CC4)nc3)ccc2N1] | 475 |
| 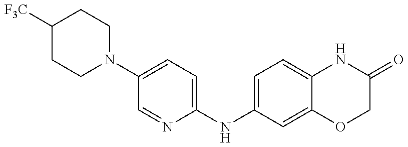 [SMILES: O=C1COc2cc(Nc3ccc(N4CCC(C(F)(F)F)CC4)cn3)ccc2N1] | 476 |
| 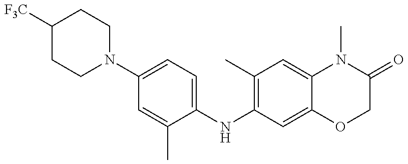 [SMILES: Cc1cc(N2CCC(C(F)(F)F)CC2)ccc1Nc1cc2c(cc1C)N(C)C(=O)CO2] | 477 |
| 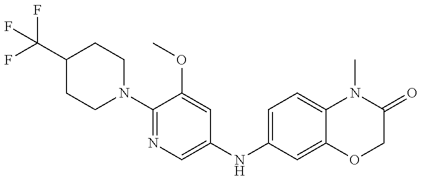 [SMILES: COc1cc(Nc2ccc3c(c2)OCC(=O)N3C)cnc1N1CCC(C(F)(F)F)CC1] | 478 |
| 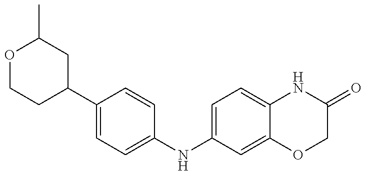 [SMILES: CC1CC(c2ccc(Nc3ccc4c(c3)OCC(=O)N4)cc2)CCO1] | 479 |
| 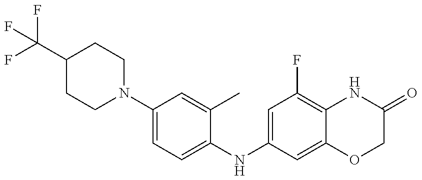 [SMILES: Cc1cc(N2CCC(C(F)(F)F)CC2)ccc1Nc1cc(F)c2c(c1)OCC(=O)N2] | 480 |
| 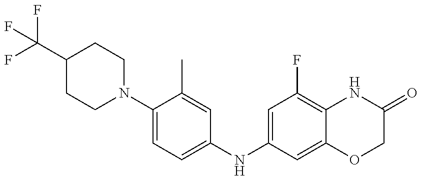 [SMILES: Cc1cc(Nc2cc(F)c3c(c2)OCC(=O)N3)ccc1N1CCC(C(F)(F)F)CC1] | 481 |
| 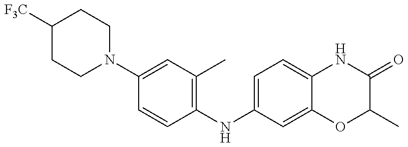 [SMILES: Cc1cc(N2CCC(C(F)(F)F)CC2)ccc1Nc1ccc2c(c1)OC(C)C(=O)N2] | 482 |
| 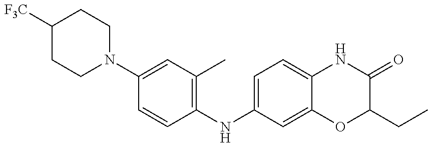 [SMILES: CCC1Oc2cc(Nc3ccc(N4CCC(C(F)(F)F)CC4)cc3C)ccc2NC1=O] | 483 |
| 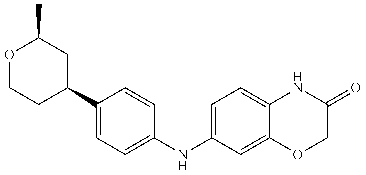 [SMILES: C[C@H]1C[C@@H](c2ccc(Nc3ccc4c(c3)OCC(=O)N4)cc2)CCO1] | 484 |
| 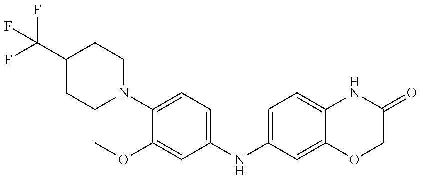 [SMILES: COc1cc(Nc2ccc3c(c2)OCC(=O)N3)ccc1N1CCC(C(F)(F)F)CC1] | 485 |
| 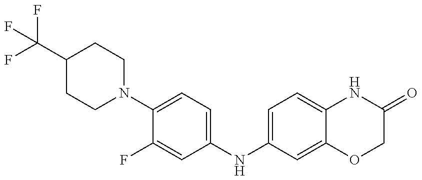 [SMILES: O=C1COc2cc(Nc3ccc(N4CCC(C(F)(F)F)CC4)c(F)c3)ccc2N1] | 486 |
| 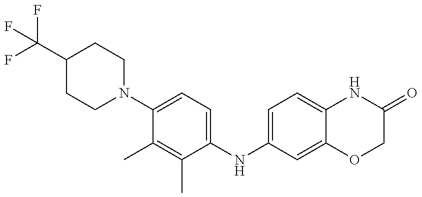 [SMILES: Cc1c(Nc2ccc3c(c2)OCC(=O)N3)ccc(N2CCC(C(F)(F)F)CC2)c1C] | 487 |
| 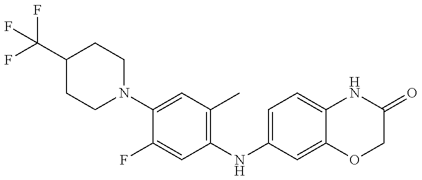 [SMILES: Cc1cc(N2CCC(C(F)(F)F)CC2)c(F)cc1Nc1ccc2c(c1)OCC(=O)N2] | 488 |
| 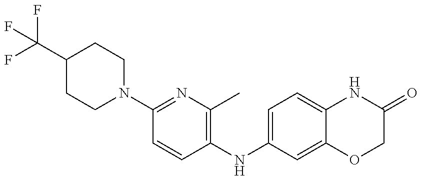 [SMILES: Cc1nc(N2CCC(C(F)(F)F)CC2)ccc1Nc1ccc2c(c1)OCC(=O)N2] | 489 |
| 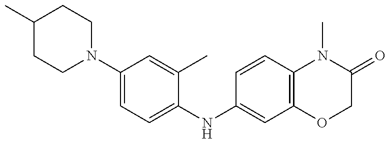 [SMILES: Cc1cc(N2CCC(C)CC2)ccc1Nc1ccc2c(c1)OCC(=O)N2C] | 490 |
| 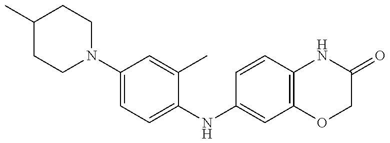 [SMILES: Cc1cc(N2CCC(C)CC2)ccc1Nc1ccc2c(c1)OCC(=O)N2] | 491 |
| 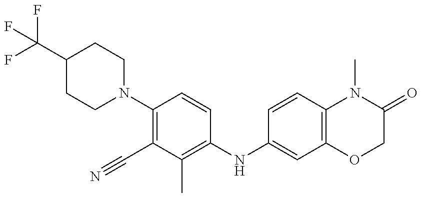 [SMILES: Cc1c(Nc2ccc3c(c2)OCC(=O)N3C)ccc(N2CCC(C(F)(F)F)CC2)c1C#N] | 492 |
| 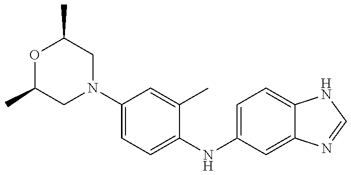 [SMILES: Cc1cc(N2C[C@@H](C)O[C@@H](C)C2)ccc1Nc1ccc2[nH]cnc2c1] | 493 |
| 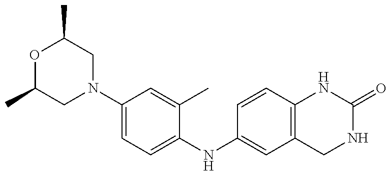 [SMILES: Cc1cc(N2C[C@@H](C)O[C@@H](C)C2)ccc1Nc1ccc2c(c1)CNC(=O)N2] | 494 |
| 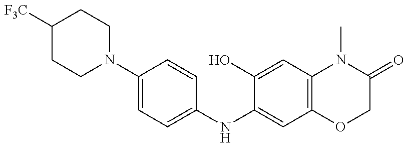 [SMILES: CN1C(=O)COc2cc(Nc3ccc(N4CCC(C(F)(F)F)CC4)cc3)c(O)cc21] | 495 |
| 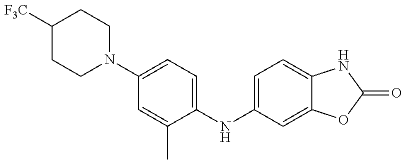 [SMILES: Cc1cc(N2CCC(C(F)(F)F)CC2)ccc1Nc1ccc2[nH]c(=O)oc2c1] | 496 |
| 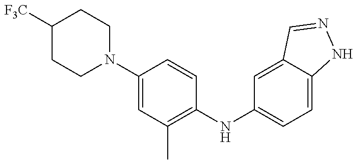 [SMILES: Cc1cc(N2CCC(C(F)(F)F)CC2)ccc1Nc1ccc2[nH]ncc2c1] | 497 |
| 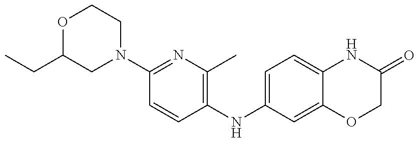 [SMILES: CCC1CN(c2ccc(Nc3ccc4c(c3)OCC(=O)N4)c(C)n2)CCO1] | 498 |
| 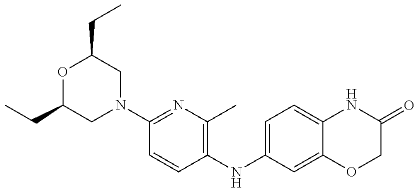 [SMILES: CC[C@@H]1CN(c2ccc(Nc3ccc4c(c3)OCC(=O)N4)c(C)n2)C[C@H](CC)O1] | 499 |
| 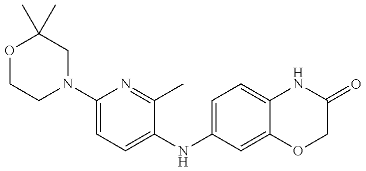 [SMILES: Cc1nc(N2CCOC(C)(C)C2)ccc1Nc1ccc2c(c1)OCC(=O)N2] | 500 |
| 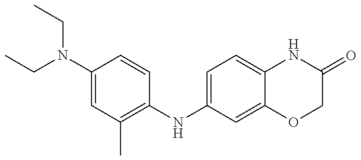 [SMILES: CCN(CC)c1ccc(Nc2ccc3c(c2)OCC(=O)N3)c(C)c1] | 501 |
| 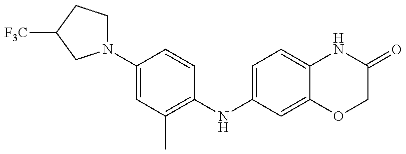 [SMILES: Cc1cc(N2CCC(C(F)(F)F)C2)ccc1Nc1ccc2c(c1)OCC(=O)N2] | 502 |
| 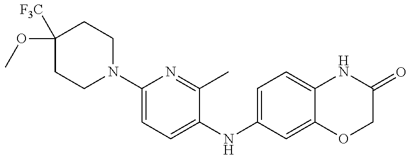 [SMILES: COC1(C(F)(F)F)CCN(c2ccc(Nc3ccc4c(c3)OCC(=O)N4)c(C)n2)CC1] | 503 |
| 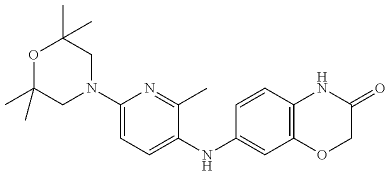 [SMILES: Cc1nc(N2CC(C)(C)OC(C)(C)C2)ccc1Nc1ccc2c(c1)OCC(=O)N2] | 504 |
| 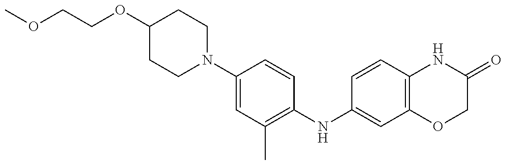 [SMILES: COCCOC1CCN(c2ccc(Nc3ccc4c(c3)OCC(=O)N4)c(C)c2)CC1] | 505 |
| 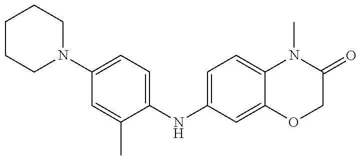 [SMILES: Cc1cc(N2CCCCC2)ccc1Nc1ccc2c(c1)OCC(=O)N2C] | 506 |
| 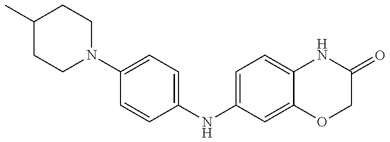 [SMILES: CC1CCN(c2ccc(Nc3ccc4c(c3)OCC(=O)N4)cc2)CC1] | 507 |
| 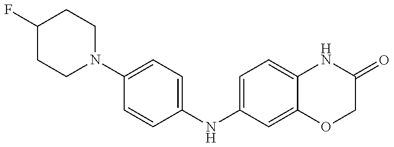 [SMILES: O=C1COc2cc(Nc3ccc(N4CCC(F)CC4)cc3)ccc2N1] | 508 |
| 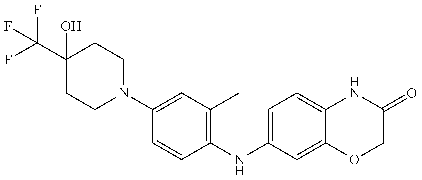 [SMILES: Cc1cc(N2CCC(O)(C(F)(F)F)CC2)ccc1Nc1ccc2c(c1)OCC(=O)N2] | 509 |
| 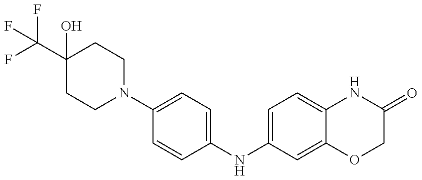 [SMILES: O=C1COc2cc(Nc3ccc(N4CCC(O)(C(F)(F)F)CC4)cc3)ccc2N1] | 510 |
| 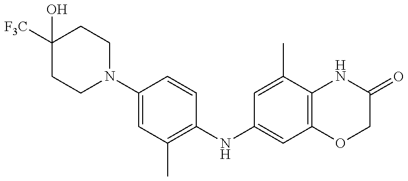 [SMILES: Cc1cc(N2CCC(O)(C(F)(F)F)CC2)ccc1Nc1cc(C)c2c(c1)OCC(=O)N2] | 511 |
| 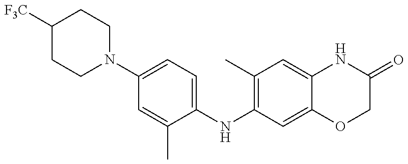 [SMILES: Cc1cc(N2CCC(C(F)(F)F)CC2)ccc1Nc1cc2c(cc1C)NC(=O)CO2] | 512 |
| 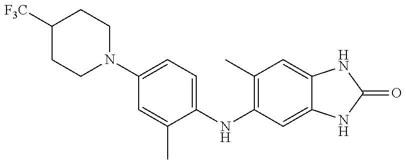 [SMILES: Cc1cc(N2CCC(C(F)(F)F)CC2)ccc1Nc1cc2[nH]c(=O)[nH]c2cc1C] | 513 |
| 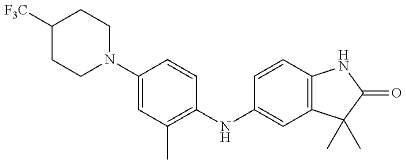 [SMILES: Cc1cc(N2CCC(C(F)(F)F)CC2)ccc1Nc1ccc2c(c1)C(C)(C)C(=O)N2] | 514 |
| 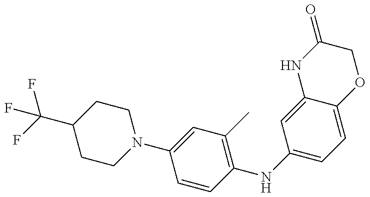 [SMILES: Cc1cc(N2CCC(C(F)(F)F)CC2)ccc1Nc1ccc2c(c1)NC(=O)CO2] | 515 |
| 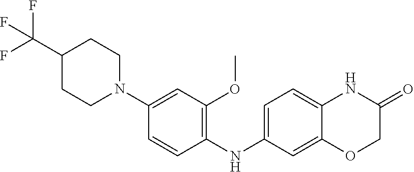 [SMILES: COc1cc(N2CCC(C(F)(F)F)CC2)ccc1Nc1ccc2c(c1)OCC(=O)N2] | 516 |
| 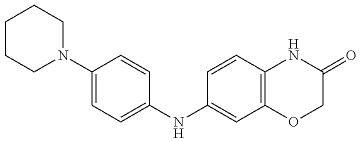 [SMILES: O=C1COc2cc(Nc3ccc(N4CCCCC4)cc3)ccc2N1] | 517 |
| 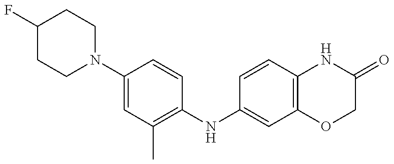 [SMILES: Cc1cc(N2CCC(F)CC2)ccc1Nc1ccc2c(c1)OCC(=O)N2] | 518 |
| 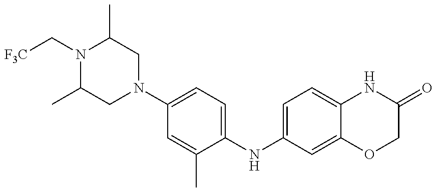 [SMILES: Cc1cc(N2CC(C)N(CC(F)(F)F)C(C)C2)ccc1Nc1ccc2c(c1)OCC(=O)N2] | 519 |
| 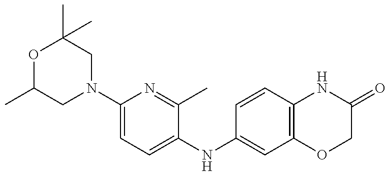 [SMILES: Cc1nc(N2CC(C)OC(C)(C)C2)ccc1Nc1ccc2c(c1)OCC(=O)N2] | 520 |
| 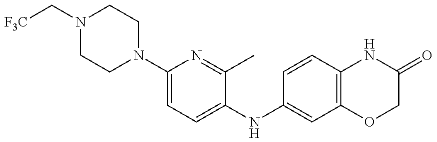 [SMILES: Cc1nc(N2CCN(CC(F)(F)F)CC2)ccc1Nc1ccc2c(c1)OCC(=O)N2] | 521 |
| 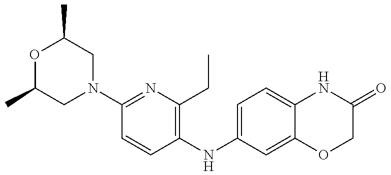 [SMILES: CCc1nc(N2C[C@@H](C)O[C@@H](C)C2)ccc1Nc1ccc2c(c1)OCC(=O)N2] | 522 |
| 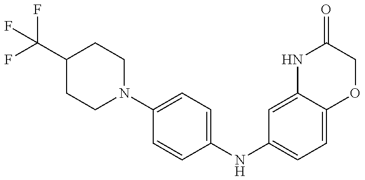 [SMILES: O=C1COc2ccc(Nc3ccc(N4CCC(C(F)(F)F)CC4)cc3)cc2N1] | 523 |
| 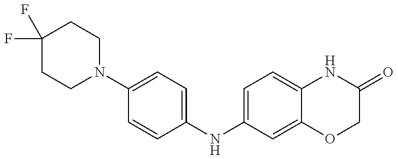 [SMILES: O=C1COc2cc(Nc3ccc(N4CCC(F)(F)CC4)cc3)ccc2N1] | 524 |
| 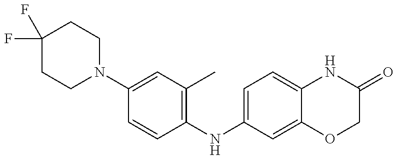 [SMILES: Cc1cc(N2CCC(F)(F)CC2)ccc1Nc1ccc2c(c1)OCC(=O)N2] | 525 |
| 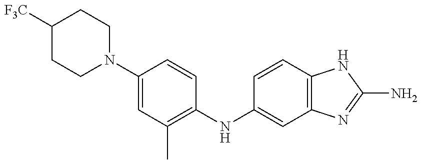 [SMILES: Cc1cc(N2CCC(C(F)(F)F)CC2)ccc1Nc1ccc2[nH]c(N)nc2c1] | 526 |
| 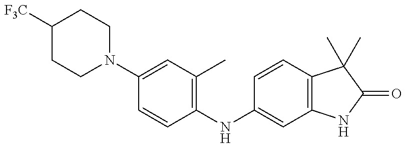 [SMILES: Cc1cc(N2CCC(C(F)(F)F)CC2)ccc1Nc1ccc2c(c1)NC(=O)C2(C)C] | 527 |
| 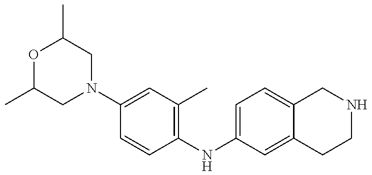 [SMILES: Cc1cc(N2CC(C)OC(C)C2)ccc1Nc1ccc2c(c1)CCNC2] | 528 |
| 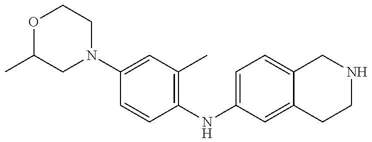 [SMILES: Cc1cc(N2CCOC(C)C2)ccc1Nc1ccc2c(c1)CCNC2] | 529 |
| 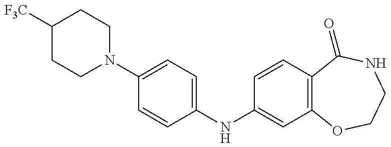 [SMILES: O=C1NCCOc2cc(Nc3ccc(N4CCC(C(F)(F)F)CC4)cc3)ccc21] | 530 |
| 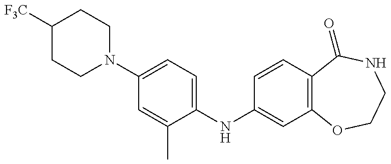 [SMILES: Cc1cc(N2CCC(C(F)(F)F)CC2)ccc1Nc1ccc2c(c1)OCCNC2=O] | 531 |
| 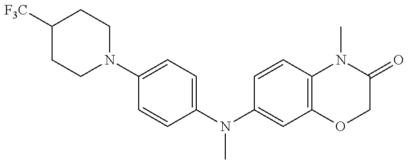 [SMILES: CN1C(=O)COc2cc(N(C)c3ccc(N4CCC(C(F)(F)F)CC4)cc3)ccc21] | 532 |
| 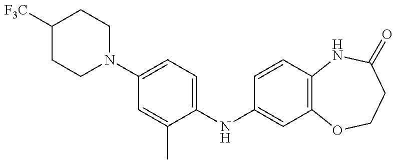 [SMILES: Cc1cc(N2CCC(C(F)(F)F)CC2)ccc1Nc1ccc2c(c1)OCCC(=O)N2] | 533 |
| 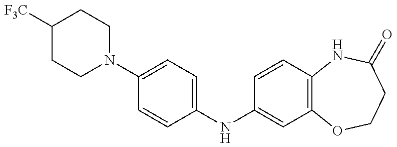 [SMILES: O=C1CCOc2cc(Nc3ccc(N4CCC(C(F)(F)F)CC4)cc3)ccc2N1] | 534 |
| 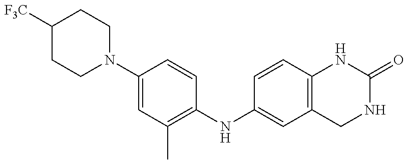 [SMILES: Cc1cc(N2CCC(C(F)(F)F)CC2)ccc1Nc1ccc2c(c1)CNC(=O)N2] | 535 |
| 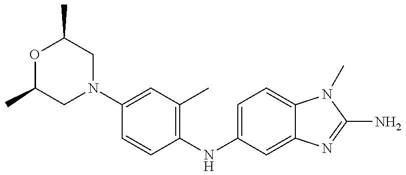 [SMILES: Cc1cc(N2C[C@@H](C)O[C@@H](C)C2)ccc1Nc1ccc2c(c1)nc(N)n2C] | 536 |
| 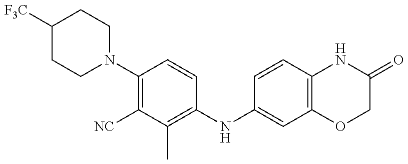 [SMILES: Cc1c(Nc2ccc3c(c2)OCC(=O)N3)ccc(N2CCC(C(F)(F)F)CC2)c1C#N] | 537 |
| 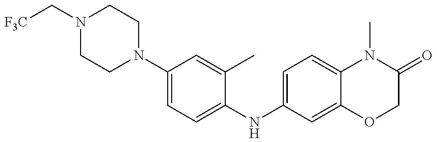 [SMILES: Cc1cc(N2CCN(CC(F)(F)F)CC2)ccc1Nc1ccc2c(c1)OCC(=O)N2C] | 538 |
| 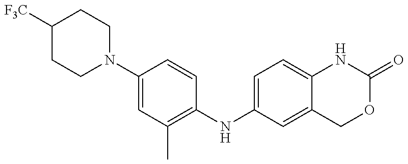 [SMILES: Cc1cc(N2CCC(C(F)(F)F)CC2)ccc1Nc1ccc2c(c1)COC(=O)N2] | 539 |
| 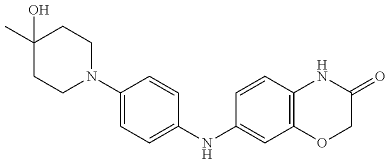 [SMILES: CC1(O)CCN(c2ccc(Nc3ccc4c(c3)OCC(=O)N4)cc2)CC1] | 540 |
| 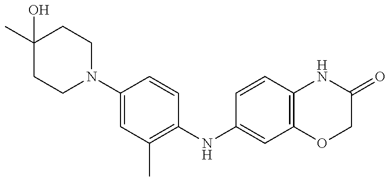 [SMILES: Cc1cc(N2CCC(C)(O)CC2)ccc1Nc1ccc2c(c1)OCC(=O)N2] | 541 |
| 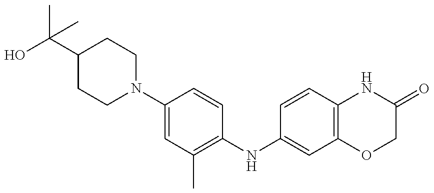 [SMILES: Cc1cc(N2CCC(C(C)(C)O)CC2)ccc1Nc1ccc2c(c1)OCC(=O)N2] | 542 |
| 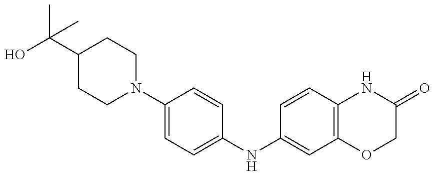 [SMILES: CC(C)(O)C1CCN(c2ccc(Nc3ccc4c(c3)OCC(=O)N4)cc2)CC1] | 543 |
| 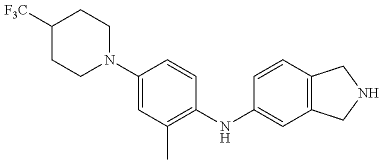 [SMILES: Cc1cc(N2CCC(C(F)(F)F)CC2)ccc1Nc1ccc2c(c1)CNC2] | 544 |
| 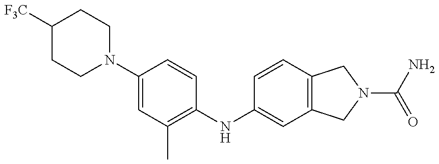 [SMILES: Cc1cc(N2CCC(C(F)(F)F)CC2)ccc1Nc1ccc2c(c1)CN(C(N)=O)C2] | 545 |
| 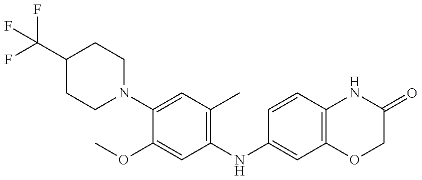 [SMILES: COc1cc(Nc2ccc3c(c2)OCC(=O)N3)c(C)cc1N1CCC(C(F)(F)F)CC1] | 546 |
| 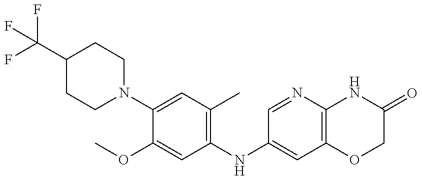 [SMILES: COc1cc(Nc2cnc3c(c2)OCC(=O)N3)c(C)cc1N1CCC(C(F)(F)F)CC1] | 547 |
| 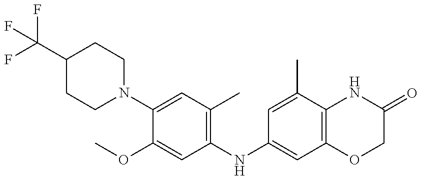 [SMILES: COc1cc(Nc2cc(C)c3c(c2)OCC(=O)N3)c(C)cc1N1CCC(C(F)(F)F)CC1] | 548 |
| 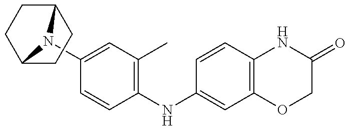 [SMILES: Cc1cc(N2[C@H]3CC[C@@H]2CC3)ccc1Nc1ccc2c(c1)OCC(=O)N2] | 549 |
| 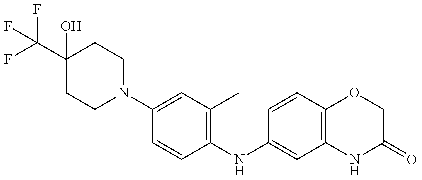 [SMILES: Cc1cc(N2CCC(O)(C(F)(F)F)CC2)ccc1Nc1ccc2c(c1)NC(=O)CO2] | 550 |
| 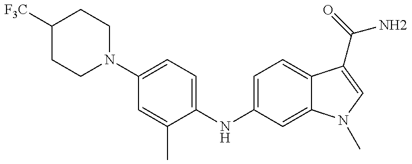 [SMILES: Cc1cc(N2CCC(C(F)(F)F)CC2)ccc1Nc1ccc2c(C(N)=O)cn(C)c2c1] | 551 |
| 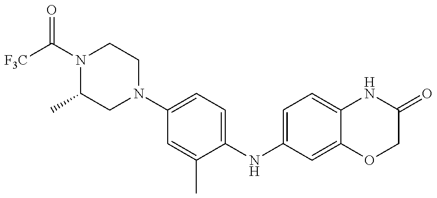 [SMILES: Cc1cc(N2CCN(C(=O)C(F)(F)F)[C@@H](C)C2)ccc1Nc1ccc2c(c1)OCC(=O)N2] | 552 |
| 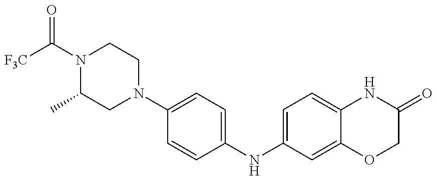 [SMILES: C[C@H]1CN(c2ccc(Nc3ccc4c(c3)OCC(=O)N4)cc2)CCN1C(=O)C(F)(F)F] | 553 |
| 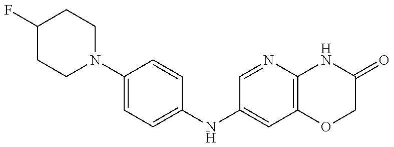 [SMILES: O=C1COc2cc(Nc3ccc(N4CCC(F)CC4)cc3)cnc2N1] | 554 |
| 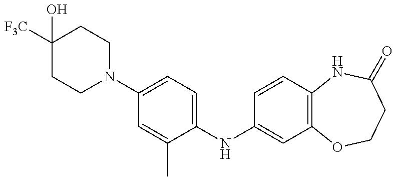 [SMILES: Cc1cc(N2CCC(O)(C(F)(F)F)CC2)ccc1Nc1ccc2c(c1)OCCC(=O)N2] | 555 |
| 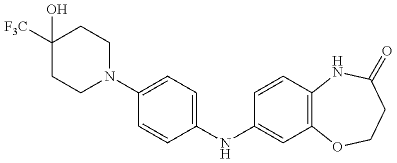 [SMILES: O=C1CCOc2cc(Nc3ccc(N4CCC(O)(C(F)(F)F)CC4)cc3)ccc2N1] | 556 |
| 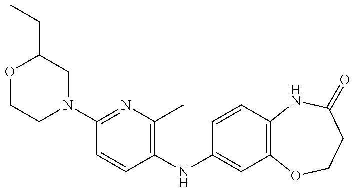 [SMILES: CCC1CN(c2ccc(Nc3ccc4c(c3)OCCC(=O)N4)c(C)n2)CCO1] | 557 |
| 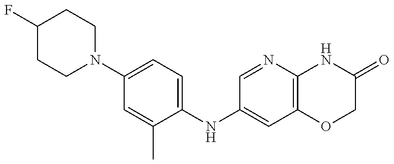 [SMILES: Cc1cc(N2CCC(F)CC2)ccc1Nc1cnc2c(c1)OCC(=O)N2] | 558 |
| 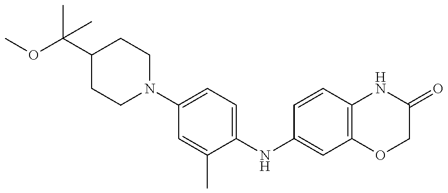 [SMILES: COC(C)(C)C1CCN(c2ccc(Nc3ccc4c(c3)OCC(=O)N4)c(C)c2)CC1] | 559 |
| 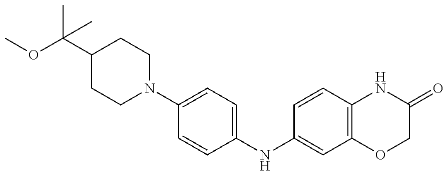 [SMILES: COC(C)(C)C1CCN(c2ccc(Nc3ccc4c(c3)OCC(=O)N4)cc2)CC1] | 560 |
| 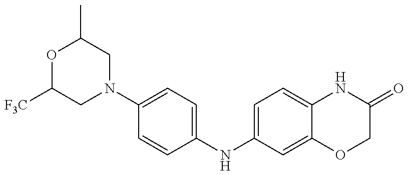 [SMILES: CC1CN(c2ccc(Nc3ccc4c(c3)OCC(=O)N4)cc2)CC(C(F)(F)F)O1] | 561 |
| 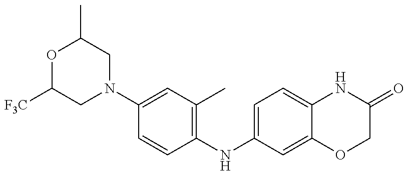 [SMILES: Cc1cc(N2CC(C)OC(C(F)(F)F)C2)ccc1Nc1ccc2c(c1)OCC(=O)N2] | 562 |
| 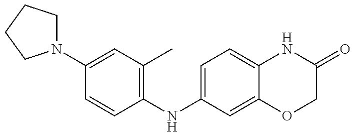 [SMILES: Cc1cc(N2CCCC2)ccc1Nc1ccc2c(c1)OCC(=O)N2] | 563 |
| 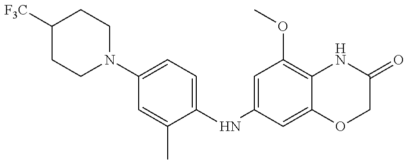 [SMILES: COc1cc(Nc2ccc(N3CCC(C(F)(F)F)CC3)cc2C)cc2c1NC(=O)CO2] | 564 |
| 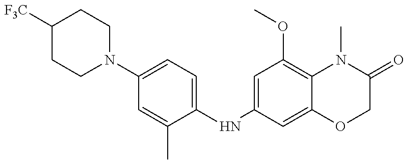 [SMILES: COc1cc(Nc2ccc(N3CCC(C(F)(F)F)CC3)cc2C)cc2c1N(C)C(=O)CO2] | 565 |
| 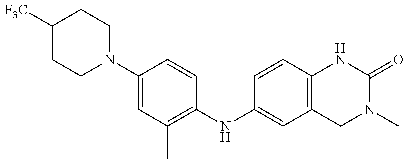 [SMILES: Cc1cc(N2CCC(C(F)(F)F)CC2)ccc1Nc1ccc2c(c1)CN(C)C(=O)N2] | 566 |
| 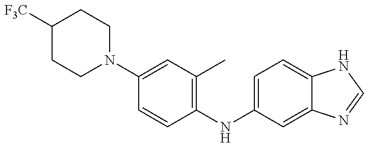 [SMILES: Cc1cc(N2CCC(C(F)(F)F)CC2)ccc1Nc1ccc2[nH]cnc2c1] | 567 |
| 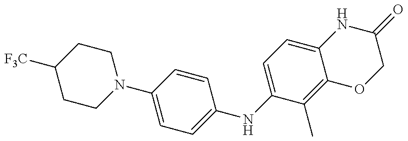 [SMILES: Cc1c(Nc2ccc(N3CCC(C(F)(F)F)CC3)cc2)ccc2c1OCC(=O)N2] | 568 |
| 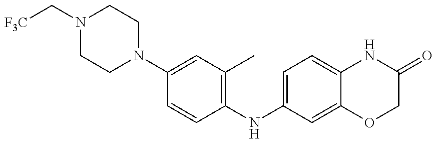 [SMILES: Cc1cc(N2CCN(CC(F)(F)F)CC2)ccc1Nc1ccc2c(c1)OCC(=O)N2] | 569 |
| 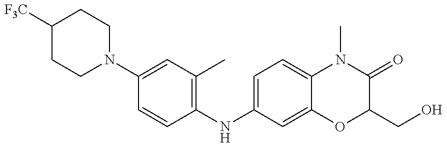 [SMILES: Cc1cc(N2CCC(C(F)(F)F)CC2)ccc1Nc1ccc2c(c1)OC(CO)C(=O)N2C] | 570 |
| 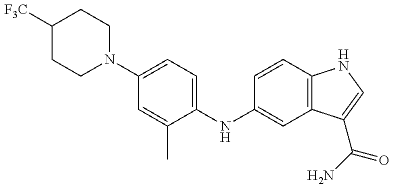 [SMILES: Cc1cc(N2CCC(C(F)(F)F)CC2)ccc1Nc1ccc2[nH]cc(C(N)=O)c2c1] | 571 |
| 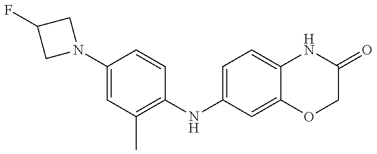 [SMILES: Cc1cc(N2CC(F)C2)ccc1Nc1ccc2c(c1)OCC(=O)N2] | 572 |
| 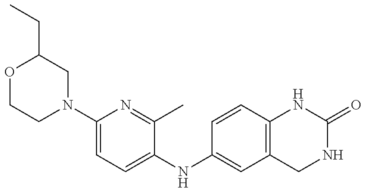 [SMILES: CCC1CN(c2ccc(Nc3ccc4c(c3)CNC(=O)N4)c(C)n2)CCO1] | 573 |
| 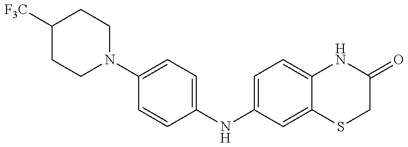 [SMILES: O=C1CSc2cc(Nc3ccc(N4CCC(C(F)(F)F)CC4)cc3)ccc2N1] | 574 |
| 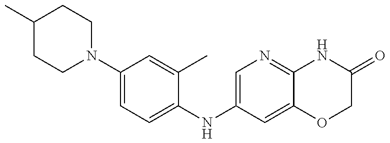 [SMILES: Cc1cc(N2CCC(C)CC2)ccc1Nc1cnc2c(c1)OCC(=O)N2] | 575 |
| 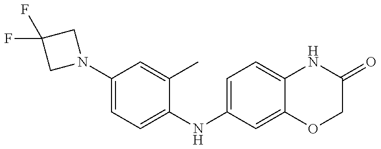 [SMILES: Cc1cc(N2CC(F)(F)C2)ccc1Nc1ccc2c(c1)OCC(=O)N2] | 576 |
| 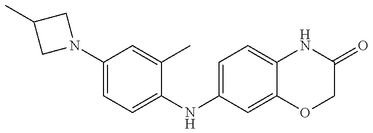 [SMILES: Cc1cc(N2CC(C)C2)ccc1Nc1ccc2c(c1)OCC(=O)N2] | 577 |
| 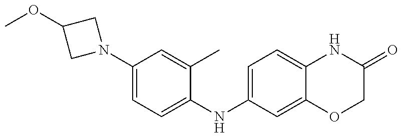 [SMILES: COC1CN(c2ccc(Nc3ccc4c(c3)OCC(=O)N4)c(C)c2)C1] | 578 |
| 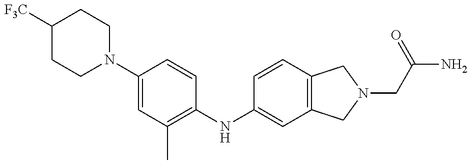 [SMILES: Cc1cc(N2CCC(C(F)(F)F)CC2)ccc1Nc1ccc2c(c1)CN(CC(N)=O)C2] | 579 |
| 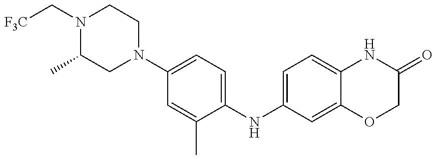 [SMILES: Cc1cc(N2CCN(CC(F)(F)F)[C@@H](C)C2)ccc1Nc1ccc2c(c1)OCC(=O)N2] | 580 |
| 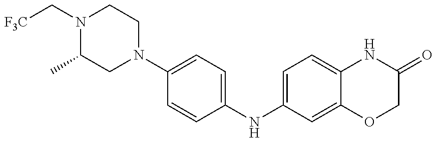 [SMILES: C[C@H]1CN(c2ccc(Nc3ccc4c(c3)OCC(=O)N4)cc2)CCN1CC(F)(F)F] | 581 |
| 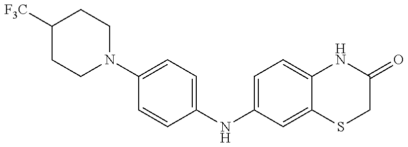 [SMILES: O=C1CSc2cc(Nc3ccc(N4CCC(C(F)(F)F)CC4)cc3)ccc2N1] | 582 |
| 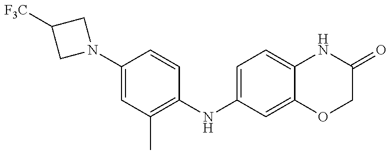 [SMILES: Cc1cc(N2CC(C(F)(F)F)C2)ccc1Nc1ccc2c(c1)OCC(=O)N2] | 583 |
| 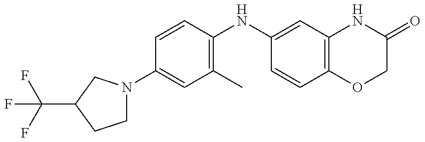 [SMILES: Cc1cc(N2CCC(C(F)(F)F)C2)ccc1Nc1ccc2c(c1)NC(=O)CO2] | 584 |
| 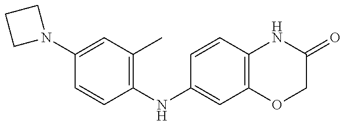 [SMILES: Cc1cc(N2CCC2)ccc1Nc1ccc2c(c1)OCC(=O)N2] | 585 |
| 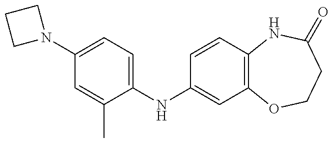 [SMILES: Cc1cc(N2CCC2)ccc1Nc1ccc2c(c1)OCCC(=O)N2] | 586 |
| 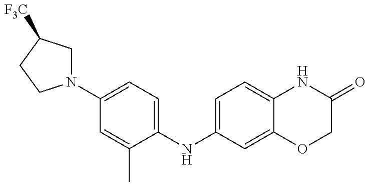 [SMILES: Cc1cc(N2CC[C@@H](C(F)(F)F)C2)ccc1Nc1ccc2c(c1)OCC(=O)N2] | 587 |
| 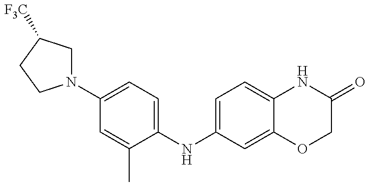 [SMILES: Cc1cc(N2CC[C@H](C(F)(F)F)C2)ccc1Nc1ccc2c(c1)OCC(=O)N2] | 588 |
| 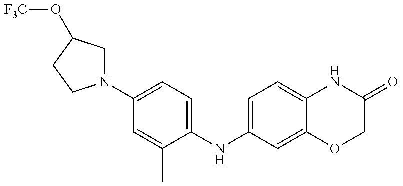 [SMILES: Cc1cc(N2CCC(OC(F)(F)F)C2)ccc1Nc1ccc2c(c1)OCC(=O)N2] | 589 |
| 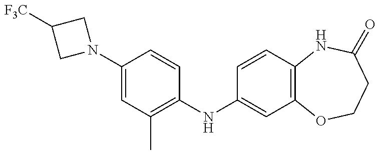 [SMILES: Cc1cc(N2CC(C(F)(F)F)C2)ccc1Nc1ccc2c(c1)OCCC(=O)N2] | 590 |
| 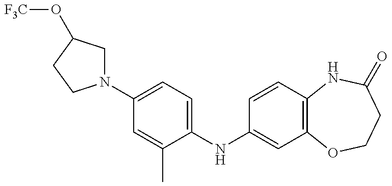 [SMILES: Cc1cc(N2CCC(OC(F)(F)F)C2)ccc1Nc1ccc2c(c1)OCCC(=O)N2] | 591 |
| 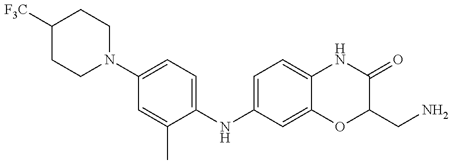 [SMILES: Cc1cc(N2CCC(C(F)(F)F)CC2)ccc1Nc1ccc2c(c1)OC(CN)C(=O)N2] | 592 |
| 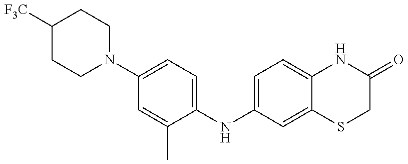 [SMILES: Cc1cc(N2CCC(C(F)(F)F)CC2)ccc1Nc1ccc2c(c1)SCC(=O)N2] | 593 |
| 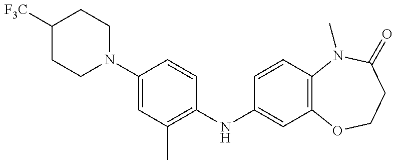 [SMILES: Cc1cc(N2CCC(C(F)(F)F)CC2)ccc1Nc1ccc2c(c1)OCCC(=O)N2C] | 594 |
| 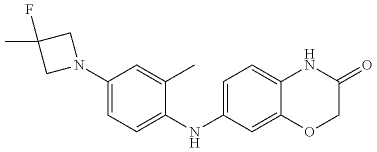 [SMILES: Cc1cc(N2CC(C)(F)C2)ccc1Nc1ccc2c(c1)OCC(=O)N2] | 595 |
| 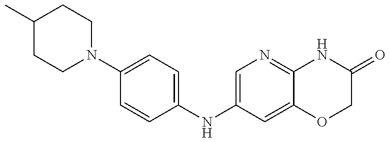 [SMILES: CC1CCN(c2ccc(Nc3cnc4c(c3)OCC(=O)N4)cc2)CC1] | 596 |
| 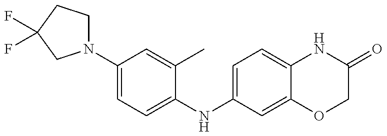 [SMILES: Cc1cc(N2CCC(F)(F)C2)ccc1Nc1ccc2c(c1)OCC(=O)N2] | 597 |
| 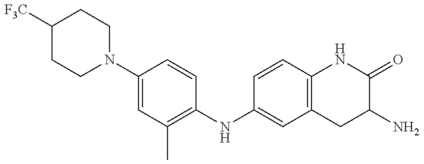 [SMILES: Cc1cc(N2CCC(C(F)(F)F)CC2)ccc1Nc1ccc2c(c1)CC(N)C(=O)N2] | 598 |
| 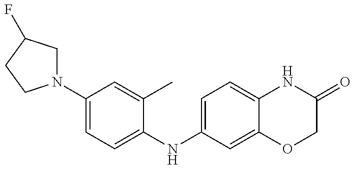 [SMILES: Cc1cc(N2CCC(F)C2)ccc1Nc1ccc2c(c1)OCC(=O)N2] | 599 |
| 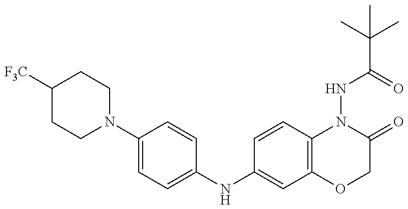 [SMILES: CC(C)(C)C(=O)NN1C(=O)COc2cc(Nc3ccc(N4CCC(C(F)(F)F)CC4)cc3)ccc21] | 600 |
| 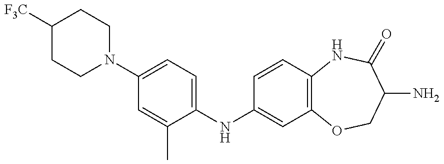 [SMILES: Cc1cc(N2CCC(C(F)(F)F)CC2)ccc1Nc1ccc2c(c1)OCC(N)C(=O)N2] | 601 |
| 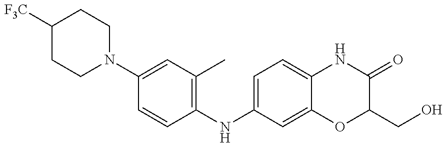 [SMILES: Cc1cc(N2CCC(C(F)(F)F)CC2)ccc1Nc1ccc2c(c1)OC(CO)C(=O)N2] | 602 |
| 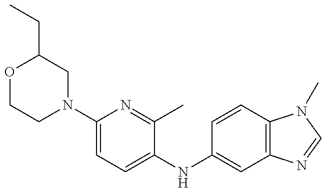 [SMILES: CCC1CN(c2ccc(Nc3ccc4c(c3)ncn4C)c(C)n2)CCO1] | 603 |
| 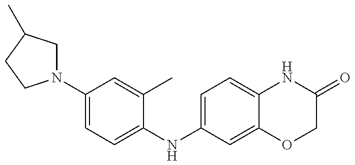 [SMILES: Cc1cc(N2CCC(C)C2)ccc1Nc1ccc2c(c1)OCC(=O)N2] | 604 |
| 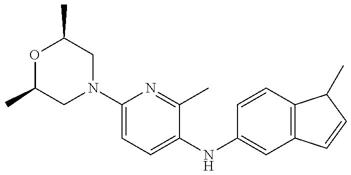 [SMILES: Cc1nc(N2C[C@@H](C)O[C@@H](C)C2)ccc1Nc1ccc2c(c1)C=CC2C] | 605 |
| 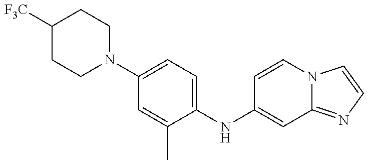 [SMILES: Cc1cc(N2CCC(C(F)(F)F)CC2)ccc1Nc1ccn2ccnc2c1] | 606 |
| 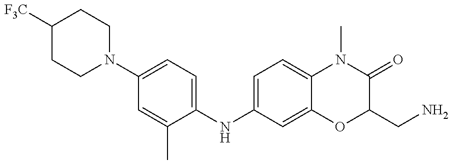 [SMILES: Cc1cc(N2CCC(C(F)(F)F)CC2)ccc1Nc1ccc2c(c1)OC(CN)C(=O)N2C] | 607 |
| 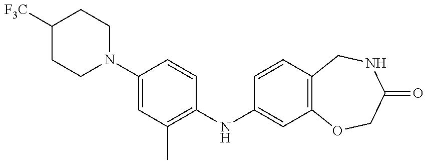 [SMILES: Cc1cc(N2CCC(C(F)(F)F)CC2)ccc1Nc1ccc2c(c1)OCC(=O)NC2] | 608 |
| 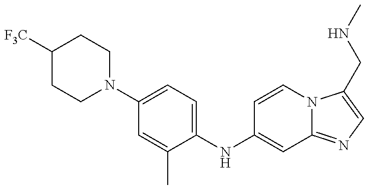 [SMILES: CNCc1cnc2cc(Nc3ccc(N4CCC(C(F)(F)F)CC4)cc3C)ccn12] | 609 |
| 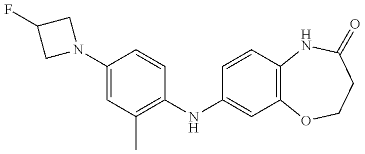 [SMILES: Cc1cc(N2CC(F)C2)ccc1Nc1ccc2c(c1)OCCC(=O)N2] | 610 |
| 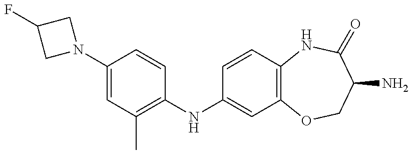 [SMILES: Cc1cc(N2CC(F)C2)ccc1Nc1ccc2c(c1)OC[C@H](N)C(=O)N2] | 611 |
| 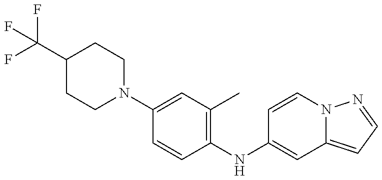 [SMILES: Cc1cc(N2CCC(C(F)(F)F)CC2)ccc1Nc1ccn2nccc2c1] | 612 |
| 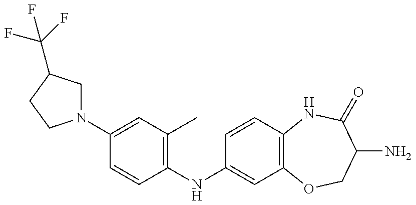 [SMILES: Cc1cc(N2CCC(C(F)(F)F)C2)ccc1Nc1ccc2c(c1)OCC(N)C(=O)N2] | 613 |
| 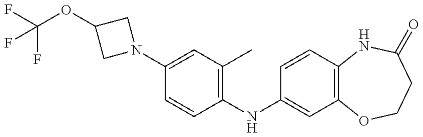 [SMILES: Cc1cc(N2CC(OC(F)(F)F)C2)ccc1Nc1ccc2c(c1)OCCC(=O)N2] | 614 |
| 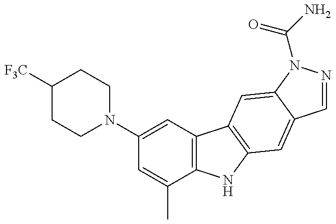 [SMILES: Cc1cc(N2CCC(C(F)(F)F)CC2)cc2c1[nH]c1cc3cnn(C(N)=O)c3cc12] | 615 |
| 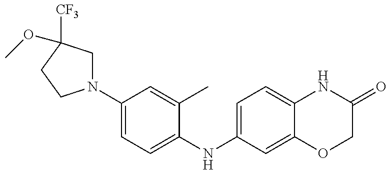 [SMILES: COC1(C(F)(F)F)CCN(c2ccc(Nc3ccc4c(c3)OCC(=O)N4)c(C)c2)C1] | 616 |
| 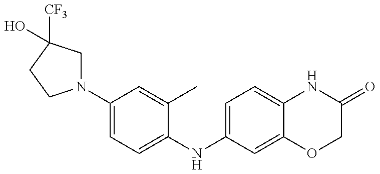 [SMILES: Cc1cc(N2CCC(O)(C(F)(F)F)C2)ccc1Nc1ccc2c(c1)OCC(=O)N2] | 617 |
| 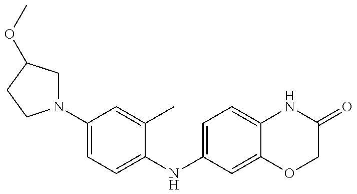 [SMILES: COC1CCN(c2ccc(Nc3ccc4c(c3)OCC(=O)N4)c(C)c2)C1] | 618 |
| 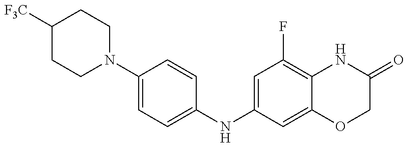 [SMILES: O=C1COc2cc(Nc3ccc(N4CCC(C(F)(F)F)CC4)cc3)cc(F)c2N1] | 619 |
| 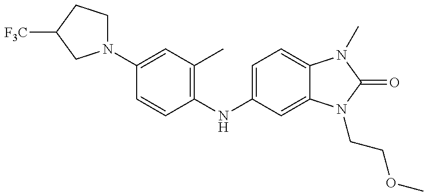 [SMILES: COCCn1c(=O)n(C)c2ccc(Nc3ccc(N4CCC(C(F)(F)F)C4)cc3C)cc21] | 620 |
| 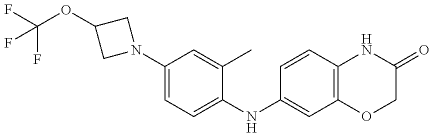 [SMILES: Cc1cc(N2CC(OC(F)(F)F)C2)ccc1Nc1ccc2c(c1)OCC(=O)N2] | 621 |
| 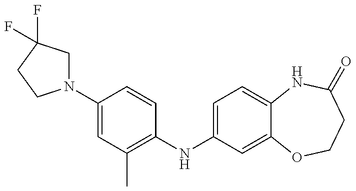 [SMILES: Cc1cc(N2CCC(F)(F)C2)ccc1Nc1ccc2c(c1)OCCC(=O)N2] | 622 |
| 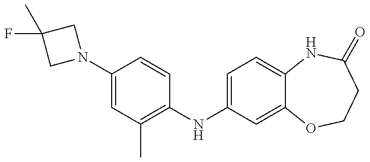 [SMILES: Cc1cc(N2CC(C)(F)C2)ccc1Nc1ccc2c(c1)OCCC(=O)N2] | 623 |
| 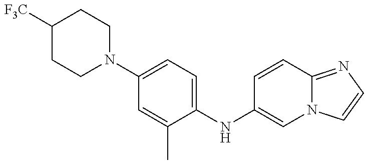 [SMILES: Cc1cc(N2CCC(C(F)(F)F)CC2)ccc1Nc1ccc2nccn2c1] | 624 |
| 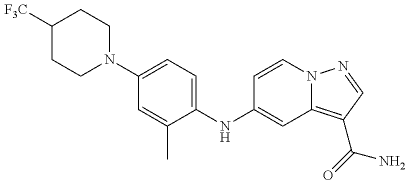 [SMILES: Cc1cc(N2CCC(C(F)(F)F)CC2)ccc1Nc1ccn2ncc(C(N)=O)c2c1] | 625 |
| 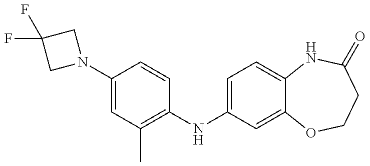 [SMILES: Cc1cc(N2CC(F)(F)C2)ccc1Nc1ccc2c(c1)OCCC(=O)N2] | 626 |
| 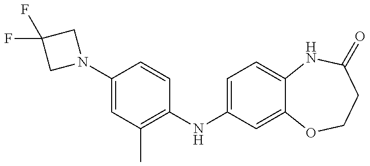 [SMILES: Cc1cc(N2CC(F)(F)C2)ccc1Nc1ccc2c(c1)OCCC(=O)N2] | 627 |
| 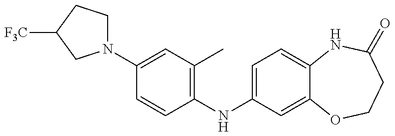 [SMILES: Cc1cc(N2CCC(C(F)(F)F)C2)ccc1Nc1ccc2c(c1)OCCC(=O)N2] | 628 |
| 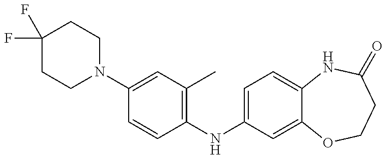 [SMILES: Cc1cc(N2CCC(F)(F)CC2)ccc1Nc1ccc2c(c1)OCCC(=O)N2] | 629 |
| 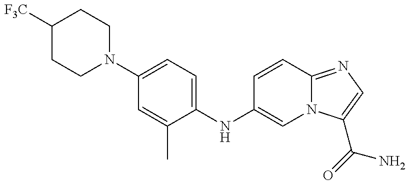 [SMILES: Cc1cc(N2CCC(C(F)(F)F)CC2)ccc1Nc1ccc2ncc(C(N)=O)n2c1] | 630 |
| 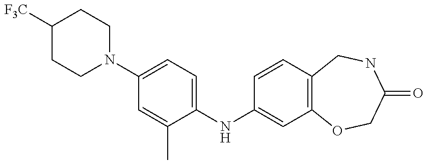 [SMILES: Cc1cc(N2CCC(C(F)(F)F)CC2)ccc1Nc1ccc2c(c1)OCC(=O)NC2] | 631 |
| 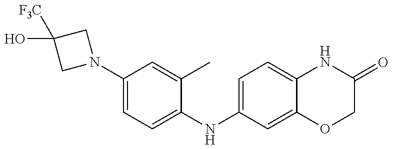 [SMILES: Cc1cc(N2CC(O)(C(F)(F)F)C2)ccc1Nc1ccc2c(c1)OCC(=O)N2] | 632 |
| 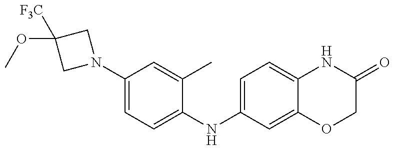 [SMILES: COC1(C(F)(F)F)CN(c2ccc(Nc3ccc4c(c3)OCC(=O)N4)c(C)c2)C1] | 633 |
| 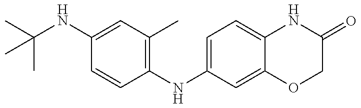 [SMILES: Cc1cc(NC(C)(C)C)ccc1Nc1ccc2c(c1)OCC(=O)N2] | 634 |
| 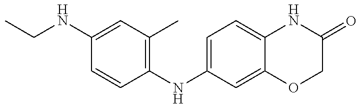 [SMILES: CCNc1ccc(Nc2ccc3c(c2)OCC(=O)N3)c(C)c1] | 635 |
| 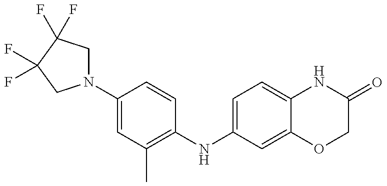 [SMILES: Cc1cc(N2CC(F)(F)C(F)(F)C2)ccc1Nc1ccc2c(c1)OCC(=O)N2] | 636 |
| 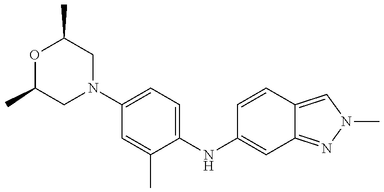 | 637 |
| 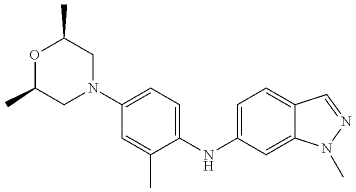 [SMILES: Cc1cc(N2C[C@@H](C)O[C@@H](C)C2)ccc1Nc1ccc2cnn(C)c2c1] | 638 |
| 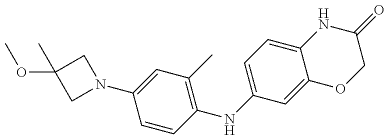 [SMILES: COC1(C)CN(c2ccc(Nc3ccc4c(c3)OCC(=O)N4)c(C)c2)C1] | 639 |
| 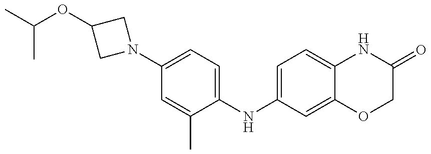 [SMILES: Cc1cc(N2CC(OC(C)C)C2)ccc1Nc1ccc2c(c1)OCC(=O)N2] | 640 |
| 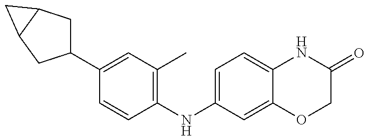 [SMILES: Cc1cc(C2CC3CC3C2)ccc1Nc1ccc2c(c1)OCC(=O)N2] | 641 |
| 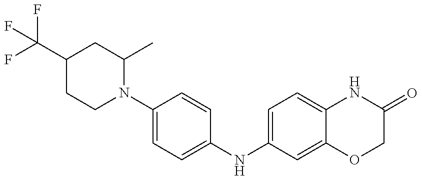 [SMILES: CC1CC(C(F)(F)F)CCN1c1ccc(Nc2ccc3c(c2)OCC(=O)N3)cc1] | 642 |
| 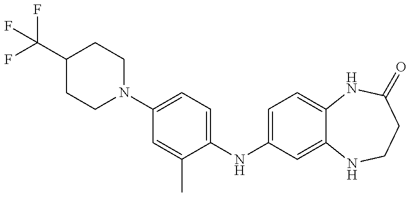 [SMILES: Cc1cc(N2CCC(C(F)(F)F)CC2)ccc1Nc1ccc2c(c1)NCCC(=O)N2] | 643 |
| 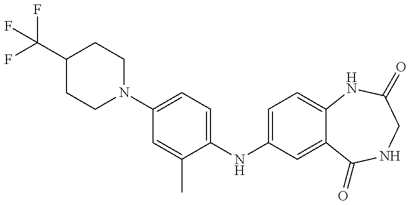 [SMILES: Cc1cc(N2CCC(C(F)(F)F)CC2)ccc1Nc1ccc2c(c1)C(=O)NCC(=O)N2] | 644 |
| 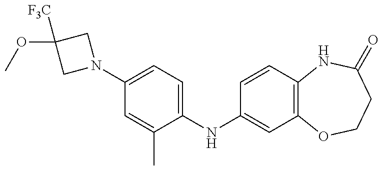 [SMILES: COC1(C(F)(F)F)CN(c2ccc(Nc3ccc4c(c3)OCCC(=O)N4)c(C)c2)C1] | 645 |
| 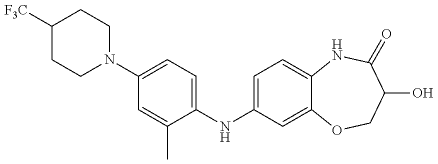 [SMILES: Cc1cc(N2CCC(C(F)(F)F)CC2)ccc1Nc1ccc2c(c1)OCC(O)C(=O)N2] | 646 |
| 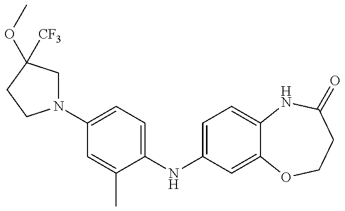 [SMILES: COC1(C(F)(F)F)CCN(c2ccc(Nc3ccc4c(c3)OCCC(=O)N4)c(C)c2)C1] | 647 |
| 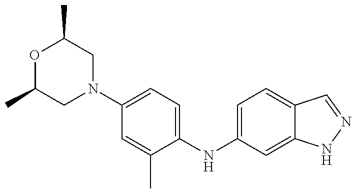 [SMILES: Cc1cc(N2C[C@@H](C)O[C@@H](C)C2)ccc1Nc1ccc2cn[nH]c2c1] | 648 |
| 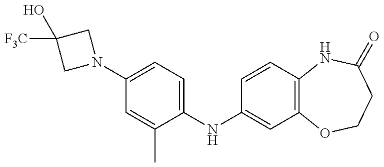 [SMILES: Cc1cc(N2CC(O)(C(F)(F)F)C2)ccc1Nc1ccc2c(c1)OCCC(=O)N2] | 649 |
| 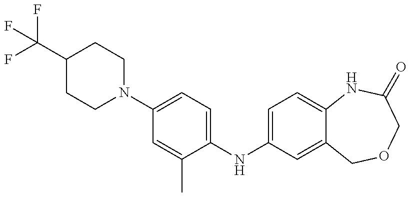 [SMILES: Cc1cc(N2CCC(C(F)(F)F)CC2)ccc1Nc1ccc2c(c1)COCC(=O)N2] | 650 |
| 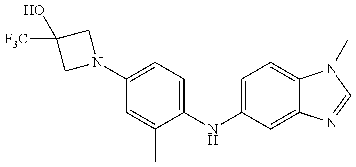 [SMILES: Cc1cc(N2CC(O)(C(F)(F)F)C2)ccc1Nc1ccc2c(c1)ncn2C] | 651 |
| 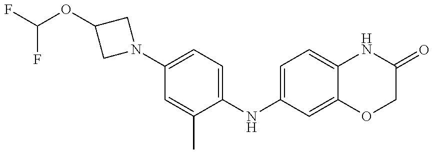 [SMILES: Cc1cc(N2CC(OC(F)F)C2)ccc1Nc1ccc2c(c1)OCC(=O)N2] | 652 |
| 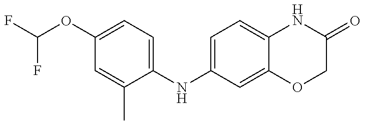 [SMILES: Cc1cc(OC(F)F)ccc1Nc1ccc2c(c1)OCC(=O)N2] | 653 |
| 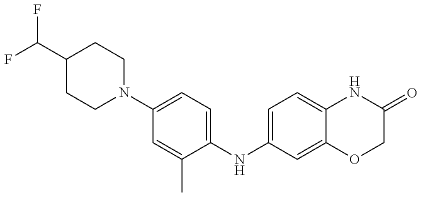 [SMILES: Cc1cc(N2CCC(C(F)F)CC2)ccc1Nc1ccc2c(c1)OCC(=O)N2] | 654 |
| 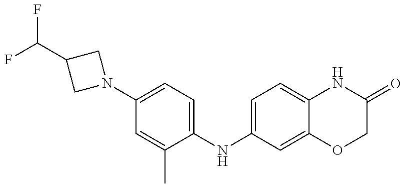 [SMILES: Cc1cc(N2CC(C(F)F)C2)ccc1Nc1ccc2c(c1)OCC(=O)N2] | 655 |
| 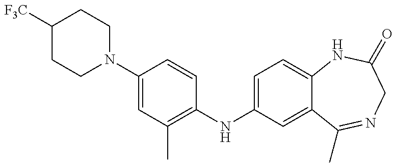 [SMILES: CC1=NCC(=O)Nc2ccc(Nc3ccc(N4CCC(C(F)(F)F)CC4)cc3C)cc21] | 656 |
| 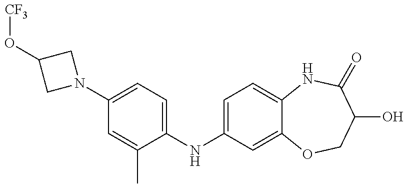 [SMILES: Cc1cc(N2CC(OC(F)(F)F)C2)ccc1Nc1ccc2c(c1)OCC(O)C(=O)N2] | 657 |
| 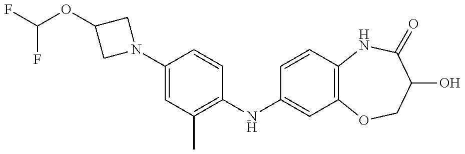 [SMILES: Cc1cc(N2CC(OC(F)F)C2)ccc1Nc1ccc2c(c1)OCC(O)C(=O)N2] | 658 |
| 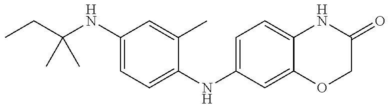 [SMILES: CCC(C)(C)Nc1ccc(Nc2ccc3c(c2)OCC(=O)N3)c(C)c1] | 659 |

Another aspect of the disclosure provides a pharmaceutical composition comprising at least one compound selected from a compound of the Formulae disclosed herein, Compounds 1 to 659, a tautomer thereof, a solvate or stereoisomer of the compound or the tautomer, or a pharmaceutically acceptable salt of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include an additional active pharmaceutical agent. Alternatively, a pharmaceutical composition comprising a compound selected from a compound of the Formulae disclosed herein, Compounds 1 to 659, a tautomer thereof, a solvate or stereoisomer of the compound or the tautomer, or a pharmaceutically acceptable salt of the foregoing can be administered as a separate composition concurrently with, prior to, or subsequent to, a composition comprising an additional active pharmaceutical agent.

In some embodiments, the pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

A compound selected from a compound of the Formulae disclosed herein, Compounds 1 to 659, a tautomer thereof, a solvate or stereoisomer of the compound or the tautomer, or a pharmaceutically acceptable salt of the foregoing, or a pharmaceutical composition disclosed herein can be administered orally in solid dosage forms, such as capsules, tablets, troches, dragées, granules and powders, or in liquid dosage forms, such as elixirs, syrups, emulsions, dispersions, and suspensions. The compound, tautomer, solvate, stereoisomer, or pharmaceutically acceptable salt described herein can also be administered parenterally, in sterile liquid dosage forms, such as dispersions, suspensions or solutions. Other dosages forms that can also be used to administer the compound, tautomer, solvate, stereoisomer, or pharmaceutically acceptable salt described herein as an ointment, cream, drops, transdermal patch or powder for topical administration, as an ophthalmic solution or suspension formation, e.g., eye drops, for ocular administration, as an aerosol spray or powder composition for inhalation or intranasal administration, or as a cream, ointment, spray or suppository for rectal or vaginal administration.

Gelatin capsules containing a compound, a tautomer thereof, a solvate or stereoisomer of the compound or the tautomer, and/or a pharmaceutically acceptable salt of the foregoing disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, a suitable oil, saline, aqueous dextrose (glucose), and related sugar solutions and glycols such as propylene glycol or polyethylene glycols can be examples of suitable carriers for parenteral solutions. Solutions for parenteral administration may comprise a water soluble salt of the at least one compound describe herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are described in Remington's Pharmaceutical Sciences, A. Osol.

For administration by inhalation, the compound, tautomer, solvate, stereoisomer, or pharmaceutically acceptable salt described herein may be conveniently delivered in the form of an aerosol spray presentation from pressurized packs or nebulisers. The compound, tautomer, solvate, stereoisomer, or pharmaceutically acceptable salt described herein may also be delivered as powders, which may be formulated, and the powder composition may be inhaled with the aid of an insufflation powder inhaler device. One exemplary delivery system for inhalation can be metered dose inhalation (MDI) aerosol, which may be formulated as a suspension or solution of a compound, tautomer, solvate, stereoisomer, or pharmaceutically acceptable salt described herein in at least one suitable propellant, selected, for example, from fluorocarbons and hydrocarbons.

For ocular administration, an ophthalmic preparation may be formulated with an appropriate weight percentage of a solution or suspension of the compound, tautomer, solvate, stereoisomer, or pharmaceutically acceptable salt described herein in an appropriate ophthalmic vehicle, such that the compound, tautomer, solvate, stereoisomer, or pharmaceutically acceptable salt described herein is maintained in contact with the ocular surface for a sufficient time period to allow the compound to penetrate the corneal and internal regions of the eye.

Useful pharmaceutical dosage-forms for administration of the compound, tautomer, solvate, stereoisomer, or pharmaceutically acceptable salt described herein include, but are not limited to, hard and soft gelatin capsules, tablets, parenteral injectables, and oral suspensions. In some embodiments, the pharmaceutical compositions disclosed herein may be in the form of controlled release or sustained release compositions as known in the art.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, lozenges or the like in the case of solid compositions. In such compositions, the active material is usually a component ranging from about 0.1 to about 50% by weight or preferably from about 1 to about 40% by weight with the remainder being various vehicles or carriers and processing aids helpful for forming the desired dosing form. Unit dosage formulations are preferably about of 5, 10, 25, 50, 100, 250, 500, or 1,000 mg per unit. In a particular embodiment, unit dosage forms are packaged in a multipack adapted for sequential use, such as blisterpack comprising sheets of at least 6, 9 or 12 unit dosage forms.

In some embodiments, unit capsules can be prepared by filling standard two-piece hard gelatin capsules each with, for example, 100 milligrams of the compound, tautomer, solvate, stereoisomer, or pharmaceutically acceptable salt described herein in powder, 150 milligrams of lactose, 50 milligrams of cellulose, and 6 milligrams magnesium stearate.

In some embodiments, a mixture of the compound, tautomer, solvate, stereoisomer, or pharmaceutically acceptable salt described herein and a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, tablets can be prepared by conventional procedures so that the dosage unit comprises, for example, 100 milligrams of the compound, stereoisomers thereof, or pharmaceutically acceptable salts thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of the compound and/or at least an enantiomer, a diastereoisomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound, stereoisomers thereof, or pharmaceutically acceptable salts thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound, tautomer, solvate, stereoisomer, or pharmaceutically acceptable salt described herein is administered stepwise or in conjunction with at least one other therapeutic agent. When drugs are administered in physical combination, the dosage form and administration route should be selected depending on the compatibility of the combined drugs. Thus, the term “coadministration” is understood to include the administration of at least two agents concomitantly or sequentially, or alternatively as a fixed dose combination of the at least two active components.

The compound, tautomer, solvate, stereoisomer, or pharmaceutically acceptable salt disclosed herein can be administered as the sole active ingredient or in combination with at least one second active ingredient.

The compound, tautomer, solvate, or stereoisomer described herein may be used per se, or in the form of their pharmaceutically acceptable salts, such as hydrochlorides, hydrobromides, acetates, sulfates, citrates, carbonates, trifluoroacetates and the like. When the compound, tautomer, solvate, stereoisomer, or pharmaceutically acceptable salt described herein contain relatively acidic functionalities, salts can be obtained by addition of the desired base, either neat or in a suitable inert solvent. Examples of pharmaceutically acceptable base addition salts include sodium, potassium, calcium, ammonium, organic amine, or magnesium salts, or the like. When the compound, tautomer, solvate, or stereoisomer described herein contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic acids, and the like. Also included are salts of amino acids such as arginate and the like, and salts of organic acids like glucuronic or galacturonic acids and the like (see, for example, Berge et al., “Pharmaceutical Salts,” Journal of Pharmaceutical Science, 1977, 66, 1-19).

Neutral forms of the pharmaceutically acceptable salt described herein may be regenerated by contacting the salt with a base or acid, and isolating the parent compound in the conventional manner.

This disclosure provides prodrugs. Prodrugs of the compound, tautomer, solvate, stereoisomer, or pharmaceutically acceptable salt described herein that readily undergo chemical changes under physiological conditions to provide the compound, tautomer, solvate, stereoisomer, or pharmaceutically acceptable salt of the present disclosure. Additionally, prodrugs can be converted to the compound, tautomer, solvate, stereoisomer, or a pharmaceutically acceptable salt of the present disclosure by chemical or biochemical methods in an ex vivo environment. For example, prodrugs can be slowly converted to the compound, tautomer, solvate, stereoisomer, or pharmaceutically acceptable salt of the present disclosure when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present disclosure which is administered as an ester (the “prodrug”), but then is metabolically hydrolyzed to the carboxylic acid, i.e. the active entity.

Certain compound, tautomer, stereoisomer, or pharmaceutically acceptable salt of the disclosure can exist in unsolvated forms as well as solvated forms, including hydrate forms. Certain compound, tautomer, solvate, stereoisomer, or pharmaceutically acceptable salt of the disclosure may exist in multiple crystalline or amorphous forms.

Certain compound, tautomer, solvate, or pharmaceutically acceptable salt in this disclosure possesses asymmetric carbon atoms (optical centers) or double bonds; the racemates, enantiomers, diastereoisomers, geometric isomers and individual isomers are all intended to be encompassed within the scope of the present disclosure.

### III. Methods of Treatment and Uses

Another aspect of the disclosure provides a method of treating a disease or condition, comprising administering to a subject in need thereof, a therapeutically effective amount of a compound of Formulae disclosed herein, a tautomer thereof, a solvate or stereoisomer of the compound or the tautomer, or a pharmaceutically acceptable salt of the foregoing, or a pharmaceutical composition comprising any of the foregoing, wherein the disease or condition is selected from neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. In some embodiments, the disease or condition is involved with dysregulated ferroptosis, e.g., abnormal ferroptosis.

In another aspect, disclosed herein is a compound, tautomer, solvate, stereoisomer, or pharmaceutically acceptable salt as described herein, including a compound of the Formulae disclosed herein, Compounds 1 to 659, a tautomer thereof, a solvate or stereoisomer of the compound or the tautomer, or a pharmaceutically acceptable salt of the foregoing, or a pharmaceutical composition thereof, for use as a medicament.

In another aspect, disclosed herein is use of a compound, tautomer, solvate, stereoisomer, or pharmaceutically acceptable salt as described herein, including a compound of the Formulae disclosed herein, Compounds 1 to 659, a tautomer thereof, a solvate or stereoisomer of the compound or the tautomer, or a pharmaceutically acceptable salt of the foregoing, or a pharmaceutical composition thereof, for the manufacture of a medicament for treating a disease or condition selected from neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. In some embodiments, the disease or condition is involved with dysregulated ferroptosis, e.g., abnormal ferroptosis.

In a further aspect of this disclosure, a compound, tautomer, solvate, stereoisomer, or pharmaceutically acceptable salt as described herein, including a compound of the Formulae disclosed herein, Compounds 1 to 659, a tautomer thereof, a solvate or stereoisomer of the compound or the tautomer, or a pharmaceutically acceptable salt of the foregoing, or a pharmaceutical composition thereof, is for use in treating a disease or condition selected from neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. In some embodiments, the disease or condition is involved with dysregulated ferroptosis, e.g., abnormal ferroptosis.

Another aspect of the disclosure provides a method of modulating, e.g., inhibiting, ferroptosis in a subject in need thereof, comprising administering to the subject, a therapeutically effective amount of a compound of Formulae disclosed herein, a tautomer thereof, a solvate or stereoisomer of the compound or the tautomer, or a pharmaceutically acceptable salt of the foregoing, or a pharmaceutical composition comprising any of the foregoing.

In another aspect, disclosed herein is use of a compound, tautomer, solvate, stereoisomer, or pharmaceutically acceptable salt as described herein, including a compound of the Formulae disclosed herein, Compounds 1 to 659, a tautomer thereof, a solvate or stereoisomer of the compound or the tautomer, or a pharmaceutically acceptable salt of the foregoing, or a pharmaceutical composition thereof, for modulating, e.g., inhibiting, ferroptosis in a subject in need thereof.

In another aspect of this disclosure, a compound, tautomer, a solvate or stereoisomer of the compound or the tautomer, or pharmaceutically acceptable salt as described herein, including a compound of the Formulae disclosed herein, Compounds 1 to 659, a tautomer thereof, a solvate or stereoisomer of the compound or the tautomer, or a pharmaceutically acceptable salt of the foregoing, or a pharmaceutical composition thereof, is for use in modulating, e.g., inhibiting, ferroptosis in a subject in need thereof by contacting the subject with the compound, tautomer, a solvate or stereoisomer of the compound or the tautomer, pharmaceutically acceptable salt, or pharmaceutical composition.

A compound of the Formulae disclosed herein, Compounds 1 to 659, a tautomer thereof, a solvate or stereoisomer of the compound or the tautomer, or a pharmaceutically acceptable salt of the foregoing, or a pharmaceutical composition thereof may be administered once daily, twice daily, or three times daily, for example, for the treatment of a disease or condition, selected from neuropathy, stroke, neurodegenerative disease (e.g., Alzheimer's disease), Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction such as acute kidney failure. In some embodiments, the disease or condition is involved with dysregulated ferroptosis, e.g., abnormal ferroptosis.

A compound of the Formulae disclosed herein, Compounds 1 to 659, a tautomer thereof, a solvate or stereoisomer of the compound or the tautomer, or a pharmaceutically acceptable salt of the foregoing, or a pharmaceutical composition thereof may be administered, for example, various manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art. Parenteral administration can be by continuous infusion over a selected period of time. Other forms of administration contemplated in this disclosure are as described in International Patent Application Nos. WO 2013/075083, WO 2013/075084, WO 2013/078320, WO 2013/120104, WO 2014/124418, WO 2014/151142, and WO 2015/023915.

The contacting is generally effected by administering to the subject an effective amount of one or more compounds, tautomers, solvates, stereoisomers, and pharmaceutically acceptable salt disclosed herein. Generally, administration is adjusted to achieve a therapeutic dosage of about 0.1 to 50, preferably 0.5 to 10, more preferably 1 to 10 mg/kg, though optimal dosages are compound specific, and generally empirically determined for each compound.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, 2 mg to 1500 mg or 5 mg to 1000 mg of a compound of the Formulae disclosed herein, Compounds 1 to 659, a tautomer thereof, a solvate or stereoisomer of the compound or the tautomer, or a pharmaceutically acceptable salt of the foregoing, or a pharmaceutical composition thereof are administered once daily, twice daily, or three times daily. The compound, tautomer, solvate, stereoisomer, or pharmaceutically acceptable salt described herein is administered for morning/daytime dosing, with off period at night.

### EXAMPLES

In order that the disclosure described herein may be more fully understood, the following examples are disclosed herein. It should be understood that these examples are for illustrative purposes only and are not to be construed as limiting this disclosure in any way.

### Example 1. Synthesis of Exemplary Compounds

The compounds of the disclosure, selected from a compound of the Formulae depicted herein, a tautomer thereof, a solvate or stereoisomer of the compound or the tautomer, or a pharmaceutically acceptable salt of the foregoing, can be made according to standard chemical practices or as illustrated herein, including the following synthetic schemes for Compounds 1 to 659 as representative examples of Formula I.

### Compound 1

### 4-methyl-7-((4-(4-methylpiperidin-1-yl)phenyl)amino)-2H-benzo[b][1,4]oxazin-3(4H)-one

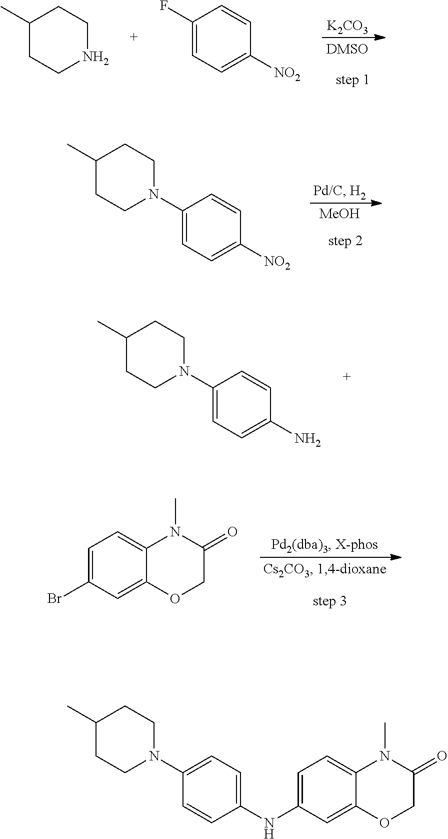

### Step 1. 4-methyl-1-(4-nitrophenyl)piperidine

To a solution of 4-methylpiperidine (252 mg, 2.55 mmol) and 1-fluoro-4-nitrobenzene (300 mg, 2.13 mmol) in DMSO (5 mL) was added K₂CO₃ (440 mg, 3.20 mmol). Then the reaction was stirred overnight at 80° C. The mixture was extracted by ethyl acetate (EA) (25 mL×3). The combined organic layers were washed with brine (15 mL×3), dried over Na₂SO₄ and concentrated to give the crude product, which was purified by silica gel chromatography (EA/petroleum ether (PE)=1:3) to give the desired product as a yellow solid (445 mg, 95%). Mass (m/z): 221.2 [M+H]⁺.

### Step 2. 4-(4-methylpiperidin-1-yl)aniline

To a solution of 4-methyl-1-(4-nitrophenyl)piperidine (445 mg, 2.02 mmol) in MeOH (10 mL) was added Pd/C (50 mg). The solution was stirred for 3 hours at room temperature (r.t.) under H₂. The reaction mixture was filtered and concentrated under vacuum to afford the desired product as a purple solid. (326 mg, 85%). Mass (m/z): 190.3 [M+H]⁺.

### Step 3. 4-methyl-7-((4-(4-methylpiperidin-1-yl)phenyl)amino)-2H-benzo[b][1,4]oxazin-3(4H)-one

To a solution of 4-(4-methylpiperidin-1-yl)aniline (49.4 mg, 0.26 mmol) and 7-bromo-4-methyl-2H-benzo[b][1,4]oxazin-3(4H)-one (48.4 mg, 0.2 mmol) in 1,4-dioxane (5 mL) was added Pd₂(dba)₃ (9.2 mg, 0.01 mmol), X-Phos (23.8 mg, 0.05 mmol) and Cs₂CO₃ (98.4 mg, 0.3 mmol) respectively under an argon atmosphere. The resulting mixture was heated to 110° C. and stirred for overnight at the same temperature. The reaction was diluted with water (10 mL) and extracted with ethyl acetate (5 mL) 3 times. The organic layer was combined and washed with water, saturated NaHCO₃ (aq) and brine respectively, and then dried over MgSO₄, filtered, and concentrated under reduced pressure. The residue was purified by silica gel column chromatography (petroleum ether/AcOEt, 1/4) to give 46.0 mg of compound 1 in a yield of 65.3% as a dark khaki powder. ¹H NMR (400 MHz, DMSO-d₆) δ 7.02-6.85 (m, 5H), 6.66-6.54 (m, 2H), 4.57 (s, 2H), 3.70-3.57 (m, 2H), 3.22 (s, 3H), 2.71-2.55 (m, 2H), 1.81-1.62 (m, 2H), 1.46 (m, 1H), 1.37-1.10 (m, 2H), 0.94 (d, J=6.4 Hz, 3H). Mass (m/z): 352.3 [M+H]⁺.

### Compound 2

### 4-methyl-7-((4-(4-(trifluoromethyl)piperidin-1-yl)phenyl)amino)-2H-benzo[b][1,4]oxazin-3(4H)-one

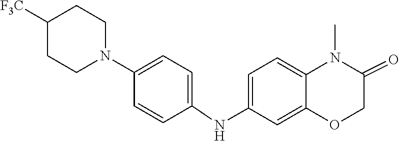 [SMILES: CN1C(=O)COc2cc(Nc3ccc(N4CCC(C(F)(F)F)CC4)cc3)ccc21]

The title compound 2 (910 mg) was prepared in a total yield of 74.6% as a yellow solid from 4-(4-(trifluoromethyl)piperidin-1-yl)aniline (878 mg, 3.6 mmol), 7-bromo-4-methyl-2H-benzo[b][1,4]oxazin-3(4H)-one (726 mg, 3.0 mmol), Pd₂(dba)₃ (27.5 mg, 0.03 mmol), X-Phos (71.55 mg, 0.15 mmol), and Cs₂CO₃ (1.47 g, 4.5 mmol) in 1,4-dioxane (30 mL) according to the procedure for compound 1. ¹H NMR (300 MHz, DMSO-d₆) δ 7.01-6.83 (m, 5H), 6.67-6.52 (m, 2H), 4.56 (s, 2H), 3.68-3.57 (m, 2H), 3.22 (s, 3H), 2.68-2.56 (m, 2H), 2.45-2.34 (m, 1H), 1.94-1.81 (m, 2H), 1.65-1.47 (m, 2H). Mass (m/z): 406.2 [M+H]⁺.

### Compound 3

### 5-methyl-8-((4-(4-(trifluoromethyl)piperidin-1-yl)phenyl)amino)-2,3-dihydrobenzo[b][1,4]oxazepin-4(5H)-one

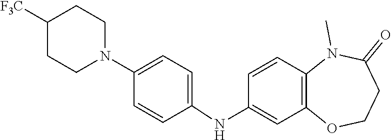 [SMILES: CN1C(=O)CCOc2cc(Nc3ccc(N4CCC(C(F)(F)F)CC4)cc3)ccc21]

The title compound 3 (9.4 mg) was prepared in a total yield of 44.9% as a sky blue solid from 4-(4-(trifluoromethyl)piperidin-1-yl)aniline (15.9 mg, 0.065 mmol), 7-bromo-4-methyl-2H-benzo[b][1,4]oxazin-3(4H)-one (12.8 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, DMSO-d₆) δ 7.14 (d, J=8.8 Hz, 1H), 7.00 (d, J=8.4 Hz, 2H), 6.91 (d, J=8.4 Hz, 2H), 6.74 (dd, J=8.8, 2.4 Hz, 1H), 6.62 (d, J=2.4 Hz, 1H), 4.40 (t, J=6.4 Hz, 2H), 3.68-3.59 (m, 2H), 3.15 (s, 3H), 2.69-2.57 (m, 2H), 2.55-2.52 (m, 2H), 2.42 (m, 1H), 1.96-1.79 (m, 2H), 1.64-1.50 (m, 2H). Mass (m/z): 420.2 [M+H]⁺.

### Compound 4

### 7-((4-((4-methoxybutyl)(pentyl)amino)phenyl)amino)-4-methyl-2H-benzo[b][1,4]oxazin-3(4H)-one

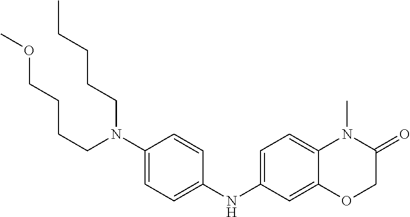 [SMILES: CCCCCN(CCCCOC)c1ccc(Nc2ccc3c(c2)OCC(=O)N3C)cc1]

The title compound 4 (17 mg) was prepared in a total yield of 21.10% as brown oil from N¹-(4-methoxybutyl)-N¹-pentylbenzene-1,4-diamine (50 mg, 0.19 mmol), 7-bromo-4-methyl-2H-benzo[b][1,4]oxazin-3(4H)-one (46 mg, 0.19 mmol), tris(dibenzylidenacetone)palladium (0) (4.5 mg, 0.009 mmol), dicyclohexyl(2′,4′,6′-triisopropyl-[1,1′-biphenyl]-2-yl)phosphane (1.73 mg, 0.019 mmol, 0.08 equivs) and sodium tert-butoxide (27 mg, 0.28 mmol, 1.5 equivs) in dry 1,4-dioxane (5 mL) according to the procedure for compound 1. ¹H NMR (400 MHz, DMSO-d₆) δ 7.57 (s, 1H), 6.99-6.85 (m, 3H), 6.64 (dd, J=22.2, 5.5 Hz, 2H), 6.52 (s, 1H), 6.44 (s, 1H), 4.54 (s, 2H), 3.21 (d, J=3.6 Hz, 1H), 1.51 (s, 7H), 1.40-1.17 (m, 3H), 0.87 (t, J=7.0 Hz, 4H). Mass (m/z): 426.7 [M+H]⁺.

### Compound 5

### 7-((4-butoxyphenyl)amino)-4-methyl-2H-benzo[b][1,4]oxazin-3(4H)-one

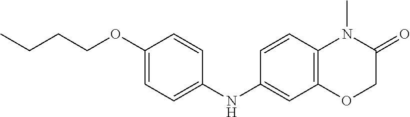 [SMILES: CCCCOc1ccc(Nc2ccc3c(c2)OCC(=O)N3C)cc1]

The title compound 5 (26.0 mg) was prepared in a total yield of 38.3% as a grey solid from 4-butoxyaniline (41 mg, 0.250 mmol), 7-bromo-4-methyl-2H-benzo[b][1,4]oxazin-3(4H)-one (50 mg, 0.208 mmol), Pd₂(dba)₃ (2 mg, 0.002 mmol), X-Phos (6 mg, 0.01 mmol) and Cs₂CO₃ (102 mg, 0.318 mmol) and 1,4-dioxane (5 mL) according to the procedure for compound 1. ¹H NMR (400 MHz, DMSO-d₆) δ 7.84 (s, 1H), 6.98 (dd, J=8.4, 5.6 Hz, 3H), 6.85 (d, J=8.8 Hz, 2H), 6.62 (dd, J=8.8, 2.4 Hz, 1H), 6.54 (d, J=2.4 Hz, 1H), 4.57 (s, 2H), 3.90 (t, J=6.4 Hz, 2H), 3.22 (s, 3H), 1.72-1.63 (m, 2H), 1.43 (h, J=7.6 Hz, 2H), 0.93 (t, J=7.6 Hz, 3H). Mass (m/z): 327.3 [M+H]⁺.

### Compound 6

### 4-methyl-7-((4-pentylphenyl)amino)-2H-benzo[b][1,4]oxazin-3(4H)-one

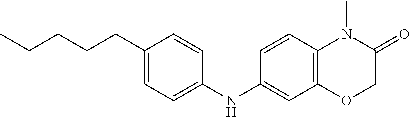 [SMILES: CCCCCc1ccc(Nc2ccc3c(c2)OCC(=O)N3C)cc1]

The title compound 6 (29.7 mg) was prepared in a total yield of 44.1% as an off-white solid from 4-pentylaniline (41 mg, 0.250 mmol), 7-bromo-4-methyl-2H-benzo[b][1,4]oxazin-3(4H)-one (50 mg, 0.208 mmol), Pd₂(dba)₃ (2 mg, 0.002 mmol), X-Phos (6 mg, 0.01 mmol) and Cs₂CO₃ (102 mg, 0.318 mmol) and 1,4-dioxane (5 mL) according to the procedure for compound 1. ¹H NMR (400 MHz, DMSO-d₆) δ 8.02 (s, 1H), 7.05 (d, J=8.4 Hz, 2H), 7.01 (d, J=8.8 Hz, 1H), 6.95 (d, J=8.4 Hz, 2H), 6.73 (dd, J=8.8, 2.4 Hz, 1H), 6.64 (d, J=2.4 Hz, 1H), 4.58 (s, 2H), 3.23 (s, 3H), 2.47 (d, J=7.6 Hz, 1H), 1.53 (p, J=7.6 Hz, 2H), 1.28 (tq, J=8.8, 5.2, 4.0 Hz, 5H), 0.86 (t, J=6.8 Hz, 3H). Mass (m/z): 325.3 [M+H]⁺.

### Compound 7

### 7-((4-(cyclohexyloxy)phenyl)amino)-4-methyl-2H-benzo[b][1,4]oxazin-3(4H)-one

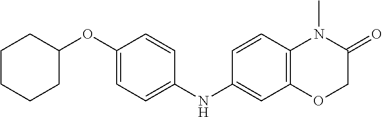 [SMILES: CN1C(=O)COc2cc(Nc3ccc(OC4CCCCC4)cc3)ccc21]

The title compound 7 (31.5 mg) was prepared in a total yield of 43.0% as a brown solid from 4-(cyclohexyloxy)aniline (48 mg, 0.250 mmol), 7-bromo-4-methyl-2H-benzo[b][1,4]oxazin-3(4H)-one (50 mg, 0.208 mmol), Pd₂(dba)₃ (2 mg, 0.002 mmol), X-phos (6 mg, 0.01 mmol), Cs₂CO₃ (106 mg, 0.312 mmol) and 1,4-dioxane (5 mL) according to the procedure for compound 1. ¹H NMR (400 MHz, DMSO-d₆) δ 7.85 (s, 1H), 6.98 (d, J=8.8 Hz, 3H), 6.85 (d, J=8.8 Hz, 2H), 6.63 (dd, J=8.8, 2.4 Hz, 1H), 6.55 (d, J=2.4 Hz, 1H), 4.57 (s, 2H), 4.19 (td, J=8.4, 4.0 Hz, 1H), 3.22 (s, 3H), 1.95-1.86 (m, 2H), 1.75-1.66 (m, 2H), 1.53 (dd, J=11.2, 5.6 Hz, 1H), 1.45-1.21 (m, 6H). Mass (m/z): 353.3 [M+H]⁺.

### Compound 8

### 4-methyl-7-((3-propoxy-4-(4-(trifluoromethyl)piperidin-1-yl)phenyl) amino)-2H-benzo[b][1,4]oxazin-3(4H)-one

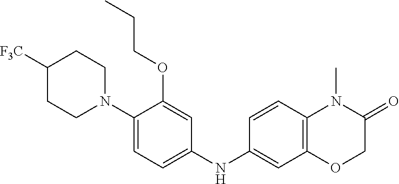 [SMILES: CCCOc1cc(Nc2ccc3c(c2)OCC(=O)N3C)ccc1N1CCC(C(F)(F)F)CC1]

The title compound 8 (28.1 mg) was prepared in a total yield of 29.3% as a brown solid from 3-propoxy-4-(4-(trifluoromethyl)piperidin-1-yl)aniline (75 mg, 0.248 mmol), 7-bromo-4-methyl-2H-benzo[b][1,4]oxazin-3(4H)-one (50 mg, 0.208 mmol), Pd₂(dba)₃ (2 mg, 0.002 mmol), X-phos (6 mg, 0.01 mmol), Cs₂CO₃ (106 mg, 0.312 mmol) and 1,4-dioxane (5 mL) according to the procedure for compound 1. ¹H NMR (300 MHz, DMSO-d₆) δ 7.91 (s, 1H), 6.98 (d, J=8.7 Hz, 1H), 6.78 (d, J=8.1 Hz, 1H), 6.69 (dd, J=8.7, 2.4 Hz, 1H), 6.61-6.54 (m, 3H), 4.57 (s, 2H), 3.85 (t, J=6.3 Hz, 2H), 3.22 (s, 3H), 2.54 (s, 1H), 2.39 (ddd, J=19.2, 9.6, 5.1 Hz, 2H), 1.87 (d, J=11.4 Hz, 2H), 1.75 (q, J=6.6 Hz, 2H), 1.58 (qd, J=12.3, 3.9 Hz, 3H), 1.02 (t, J=7.2 Hz, 3H). Mass (m/z): 464.3 [M+H]⁺.

### Compound 9

### 7-((4-(4-methoxy-4-(trifluoromethyl)piperidin-1-yl)phenyl)amino)-4-methyl-2H-benzo[b][1,4]oxazin-3(4H)-one

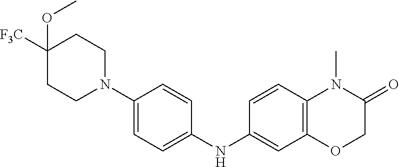 [SMILES: COC1(C(F)(F)F)CCN(c2ccc(Nc3ccc4c(c3)OCC(=O)N4C)cc2)CC1]

The title compound 9 (50.7 mg) was prepared in a total yield of 56.3% as a green solid from 4-(4-methoxy-4-(trifluoromethyl)piperidin-1-yl)aniline (68 mg, 0.248 mmol), 7-bromo-4-methyl-2H-benzo[b][1,4] oxazin-3(4H)-one (50 mg, 0.208 mmol), Pd₂(dba)₃ (2 mg, 0.002 mmol), X-phos (6 mg, 0.01 mmol) and Cs₂CO₃ (106 mg, 0.312 mmol) and 1,4-dioxane (5 mL) according to the procedure for compound 1. ¹H NMR (400 MHz, DMSO-d₆) δ 8.04 (s, 1H), 7.27-6.83 (m, 5H), 6.65 (d, J=34.0 Hz, 2H), 4.58 (s, 2H), 3.50 (s, 2H), 3.41 (d, J=1.6 Hz, 3H), 3.23 (s, 3H), 2.99 (s, 2H), 2.16-1.91 (m, 4H). Mass (m/z): 436.3 [M+H]⁺.

### Compound 10

### 4-(3-methoxypropyl)-7-((4-(4-(trifluoromethyl)piperidin-1-yl)phenyl) amino)-2H-benzo[b][1,4]oxazin-3(4H)-one

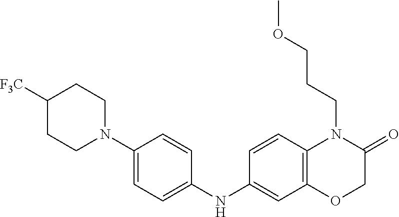 [SMILES: COCCCN1C(=O)COc2cc(Nc3ccc(N4CCC(C(F)(F)F)CC4)cc3)ccc21]

The title compound 10 (30.4 mg) was prepared in a total yield of 39.3% as a brown solid from 4-(4-(trifluoromethyl)piperidin-1-yl)aniline (49 mg, 0.200 mmol), 7-bromo-4-(3-methoxypropyl)-2H-benzo[b][1,4]oxazin-3(4H)-one (50 mg, 0.167 mmol), Pd₂(dba)₃ (2 mg, 0.002 mmol), X-Phos (6 mg, 0.01 mmol) and Cs₂CO₃ (82 mg, 0.251 mmol) and 1,4-dioxane (5 mL) according to the procedure for compound 1. ¹H NMR (400 MHz, DMSO-d₆) δ 7.92 (s, 1H), 7.02-6.94 (m, 3H), 6.92-6.87 (m, 2H), 6.63 (dd, J=8.8, 2.4 Hz, 1H), 6.56 (d, J=2.4 Hz, 1H), 4.54 (s, 2H), 3.91-3.83 (m, 2H), 3.62 (d, J=12.0 Hz, 2H), 3.34 (d, J=6.0 Hz, 2H), 3.23 (s, 3H), 2.62 (td, J=12.4, 2.4 Hz, 2H), 2.42 (ddt, J=16.0, 7.6, 4.0 Hz, 1H), 1.88 (d, J=12.4 Hz, 2H), 1.82-1.71 (m, 2H), 1.61-1.50 (m, 2H). Mass (m/z): 464.3 [M+H]⁺.

### Compound 11

### 4-(2-methoxyethyl)-7-((4-(4-(trifluoromethyl)piperidin-1-yl)phenyl) amino)-2H-benzo[b][1,4]oxazin-3(4H)-one

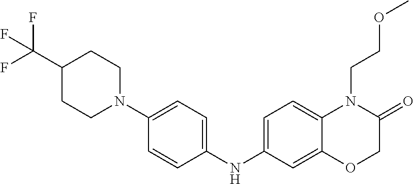 [SMILES: COCCN1C(=O)COc2cc(Nc3ccc(N4CCC(C(F)(F)F)CC4)cc3)ccc21]

The title compound 11 (53.7 mg) was prepared in a total yield of 68.3% as a brown solid from 4-(4-(trifluoromethyl)piperidin-1-yl)aniline (51 mg, 0.210 mmol), 7-bromo-4-(2-methoxyethyl)-2H-benzo[b][1,4]oxazin-3(4H)-one (50 mg, 0.175 mmol), Pd₂(dba)₃ (2 mg, 0.002 mmol), X-Phos (6 mg, 0.01 mmol) and Cs₂CO₃ (86 mg, 0.262 mmol) and 1,4-dioxane (5 mL) according to the procedure for compound 1. ¹H NMR (400 MHz, DMSO-d₆) δ 7.96 (s, 1H), 7.07 (d, J=8.8 Hz, 1H), 7.00-6.95 (m, 2H), 6.92-6.87 (m, 2H), 6.63 (dd, J=8.8, 2.4 Hz, 1H), 6.57 (d, J=2.4 Hz, 1H), 4.55 (s, 2H), 4.02 (t, J=5.6 Hz, 2H), 3.67-3.57 (m, 2H), 3.50 (t, J=5.6 Hz, 2H), 3.24 (s, 3H), 2.62 (td, J=12.4, 2.4 Hz, 2H), 2.42 (tp, J=12.4, 4.0 Hz, 1H), 1.92-1.83 (m, 2H), 1.57 (qd, J=12.4, 4.0 Hz, 2H). Mass (m/z): 450.3 [M+H]⁺.

### Compound 12

### 7-((6-butoxypyridin-3-yl)amino)-4-methyl-2H-benzo[b][1,4]oxazin-3(4H)-one

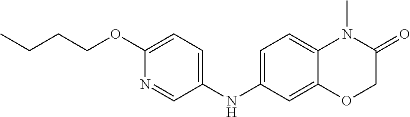 [SMILES: CCCCOc1ccc(Nc2ccc3c(c2)OCC(=O)N3C)cn1]

The title compound 12 (9.6 mg) was prepared in a yield of 14.2% as a pale yellow powder from 6-butoxypyridin-3-amine (50 mg, 0.21 mmol) and 7-bromo-4-methyl-2H-benzo[b][1,4]oxazin-3(4H)-one (38 mg, 0.24 mmol) according to the procedure for compound 1. ¹H NMR (400 MHz, DMSO-d₆) δ 7.93 (s, 1H), 7.90 (d, J=2.8 Hz, 1H), 7.46 (dd, J=8.8, 2.9 Hz, 1H), 6.99 (d, J=8.7 Hz, 1H), 6.74 (d, J=8.8 Hz, 1H), 6.60 (dd, J=8.7, 2.5 Hz, 1H), 6.51 (d, J=2.5 Hz, 1H), 4.57 (s, 2H), 4.18 (t, J=6.6 Hz, 2H), 3.22 (s, 3H), 1.67 (dq, J=8.6, 6.7 Hz, 2H), 1.51-1.32 (m, 2H), 0.92 (t, J=7.4 Hz, 3H). Mass (m/z): 328.5 [M+H]⁺.

### Compound 13

### 7-((4-(3-fluoropropoxy)phenyl)amino)-4-methyl-2H-benzo[b][1,4]oxazin-3(4H)-one

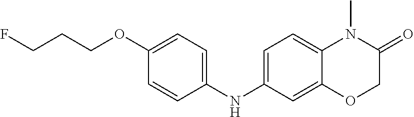 [SMILES: CN1C(=O)COc2cc(Nc3ccc(OCCCF)cc3)ccc21]

The title compound 13 (57.2 mg) was prepared in a yield of 48.83% as a brown oil from 4-(3-fluoropropoxy)aniline (60 mg, 0.35 mmol) and 7-bromo-4-methyl-2H-benzo[b][1,4]oxazin-3(4H)-one (86 mg, 0.35 mmol) according to the procedure for compound 1. ¹H NMR (400 MHz, DMSO-d₆) δ 7.85 (s, 1H), 6.98 (t, J=8.5 Hz, 3H), 6.91-6.83 (m, 2H), 6.64-6.60 (m, 1H), 6.54 (d, J=2.4 Hz, 1H), 4.65 (q, J=5.8 Hz, 1H), 4.56 (d, J=2.2 Hz, 2H), 4.52 (dd, J=11.5, 5.9 Hz, 1H), 4.00 (t, J=6.3 Hz, 2H), 3.22 (s, 3H), 2.14-2.01 (m, 2H). Mass (m/z): 331.2 [M+H]⁺.

### Compound 14

### 2-(3-oxo-7-((4-(4-(trifluoromethyl)piperidin-1-yl)phenyl)amino)-2,3-dihydro-4H-benzo[b][1,4]oxazin-4-yl)acetamide

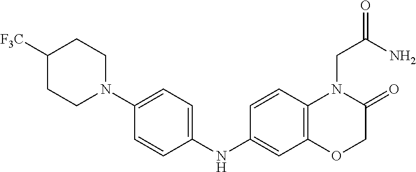 [SMILES: NC(=O)CN1C(=O)COc2cc(Nc3ccc(N4CCC(C(F)(F)F)CC4)cc3)ccc21]

The title compound 14 (12.6 mg) was prepared in a total yield of 56.10% as a light gray solid from 4-(4-(trifluoromethyl)piperidin-1-yl)aniline (15.9 mg, 0.065 mmol), 2-(7-bromo-3-oxo-2,3-dihydro-4H-benzo[b][1,4]oxazin-4-yl)acetamide (14.3 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, DMSO-d₆) δ 7.00-6.72 (m, 5H), 6.63-6.51 (m, 2H), 4.61 (s, 2H), 4.38 (s, 2H), 3.66-3.57 (m, 2H), 2.68-2.56 (m, 2H), 2.41 (m, 1H), 1.92-1.82 (m, 2H), 1.63-1.50 (m, 2H). Mass (m/z): 449.2 [M+H]⁺.

### Compound 15

### 3-(3-oxo-7-((4-(4-(trifluoromethyl)piperidin-1-yl)phenyl)amino)-2,3-dihydro-4H-benzo[b][1,4]oxazin-4-yl)propanamide

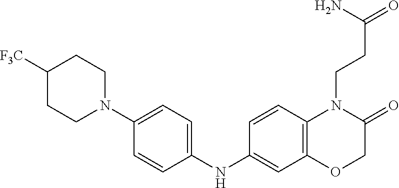 [SMILES: NC(=O)CCN1C(=O)COc2cc(Nc3ccc(N4CCC(C(F)(F)F)CC4)cc3)ccc21]

The title compound 15 (20.1 mg) was prepared in a total yield of 87.0% as a rosy brown solid from 4-(4-(trifluoromethyl)piperidin-1-yl)aniline (15.9 mg, 0.065 mmol), 3-(7-bromo-3-oxo-2,3-dihydro-4H-benzo[b][1,4] oxazin-4-yl)propanamide (14.9 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, DMSO-d₆) δ 7.04-6.86 (m, 5H), 6.65-6.53 (m, 2H), 4.54 (s, 2H), 4.05 (t, J=7.6 Hz, 2H), 3.66-3.58 (m, 2H), 2.67-2.55 (m, 2H), 2.45-2.30 (m, 3H), 1.95-1.79 (m, 2H), 1.63-1.50 (m, 2H). Mass (m/z): 463.3 [M+H]⁺.

### Compound 16

### 4-ethyl-7-((4-(4-(trifluoromethyl)piperidin-1-yl)phenyl)amino)-2H-benzo[b][1,4]oxazin-3(4H)-one

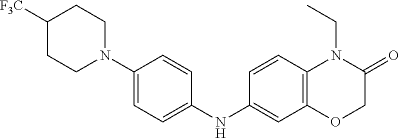 [SMILES: CCN1C(=O)COc2cc(Nc3ccc(N4CCC(C(F)(F)F)CC4)cc3)ccc21]

The title compound 16 (71.5 mg) was prepared in a total yield of 73.0% as a white solid from 4-(4-(trifluoromethyl)piperidin-1-yl)aniline (57 mg, 0.234 mmol), 7-bromo-4-ethyl-2H-benzo[b][1,4]oxazin-3(4H)-one (50 mg, 0.195 mmol), Pd₂(dba)₃ (2 mg, 0.002 mmol), X-phos (6 mg, 0.01 mmol) and Cs₂CO₃ (96 mg, 0.292 mmol) and 1,4-dioxane (5 mL) according to the procedure for compound 1. ¹H NMR (400 MHz, DMSO-d₆) δ 7.83 (s, 1H), 7.01 (d, J=8.8 Hz, 1H), 6.99-6.94 (m, 2H), 6.92-6.88 (m, 2H), 6.63 (dd, J=8.8, 2.4 Hz, 1H), 6.55 (d, J=2.4 Hz, 1H), 4.54 (s, 2H), 3.86 (q, J=7.2 Hz, 2H), 3.62 (d, J=12.0 Hz, 2H), 2.62 (td, J=12.4, 2.4 Hz, 2H), 2.42 (qd, J=10.4, 8.8, 6.0 Hz, 1H), 1.88 (dt, J=10.4, 3.2 Hz, 2H), 1.57 (qd, J=12.4, 4.0 Hz, 2H), 1.13 (t, J=7.2 Hz, 3H). Mass (m/z): 420.3 [M+H]⁺.

### Compound 17

### 4-methyl-7-((6-(4-(trifluoromethyl)piperidin-1-yl)pyridin-3-yl)amino)-2H-benzo[b][1,4]oxazin-3(4H)-one

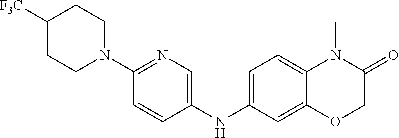 [SMILES: CN1C(=O)COc2cc(Nc3ccc(N4CCC(C(F)(F)F)CC4)nc3)ccc21]

The title compound 17 (17.1 mg) was prepared in a total yield of 84.0% as a light orange solid from 6-(4-(trifluoromethyl)piperidin-1-yl)pyridin-3-amine (15.9 mg, 0.065 mmol), 7-bromo-4-methyl-2H-benzo[b][1,4]oxazin-3(4H)-one (12.1 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, DMSO-d₆) δ 7.76 (s, 1H), 7.40-7.33 (m, 1H), 6.95 (d, J=8.8 Hz, 1H), 6.85 (d, J=8.8 Hz, 1H), 6.53 (dd, J=8.8, 2.4 Hz, 1H), 6.45 (d, J=2.4 Hz, 1H), 4.55 (s, 2H), 4.36-4.18 (m, 2H), 3.21 (s, 3H), 2.81-2.72 (m, 2H), 2.54 (m, 1H), 1.90-1.78 (m, 2H), 1.51-1.38 (m, 2H). Mass (m/z): 407.2 [M+H]⁺.

### Compound 18

### 2,2-dimethyl-1-(7-((4-(4-(trifluoromethyl)piperidin-1-yl)phenyl)amino)-2,3-dihydro-4H-benzo[b][1,4]oxazin-4-yl)propan-1-one

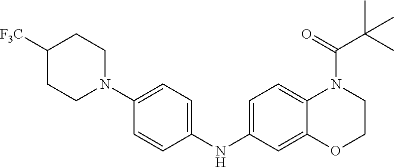 [SMILES: CC(C)(C)C(=O)N1CCOc2cc(Nc3ccc(N4CCC(C(F)(F)F)CC4)cc3)ccc21]

The title compound 18 (18.7 mg) was prepared in a total yield of 81.1% as a light gray solid from 4-(4-(trifluoromethyl)piperidin-1-yl)aniline (15.9 mg, 0.065 mmol), 1-(7-bromo-2,3-dihydro-4H-benzo[b][1,4]oxazin-4-yl)-2,2-dimethylpropan-1-one (14.9 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, DMSO-d₆) δ 7.35 (d, J=8.8 Hz, 1H), 7.02-6.83 (m, 4H), 6.48-6.33 (m, 2H), 4.21 (t, J=4.4 Hz, 2H), 3.92 (t, J=4.4 Hz, 2H), 3.67-3,55 (m, 2H), 2.69-2.54 (m, 2H), 2.41 (m, 1H), 1.93-1.82 (m, 2H), 1.63-1.50 (m, 2H), 1.27 (s, 9H). Mass (m/z): 462.2 [M+H]⁺.

### Compound 19

### 7-((4-(2,6-dimethylmorpholino)-3-(methylamino)phenyl)amino)-4-methyl-2H-benzo[b][1,4]oxazin-3(4H)-one

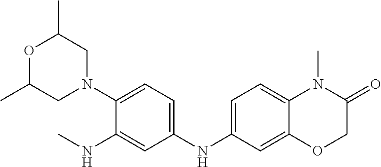 [SMILES: CNc1cc(Nc2ccc3c(c2)OCC(=O)N3C)ccc1N1CC(C)OC(C)C1]

The title compound 19 (9.3 mg) was prepared in a total yield of 46.9% as a yellow solid from 6-(2,6-dimethylmorpholino)-N¹-methylbenzene-1,3-diamine (15.3 mg, 0.065 mmol), 1-(7-bromo-2,3-dihydro-4H-benzo[b][1,4]oxazin-4-yl)-2,2-dimethylpropan-1-one (14.9 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, DMSO-d₆) δ 6.98 (d, J=8.8 Hz, 1H), 6.82-6.59 (m, 3H), 6.35-6.15 (m, 2H), 4.57 (s, 2H), 3.83-3.72 (m, 2H), 3.22 (s, 3H), 2.81-2.74 (m, 2H), 2.69 (d, J=5.2 Hz, 3H), 2.30-2.15 (m, 2H), 1.08 (d, J=6.4 Hz, 6H). Mass (m/z): 397.2 [M+H]⁺.

### Compound 20

### 7-((3-(dimethylamino)-4-(2,6-dimethylmorpholino)phenyl)amino)-4-methyl-2H-benzo[b][1,4]oxazin-3(4H)-one

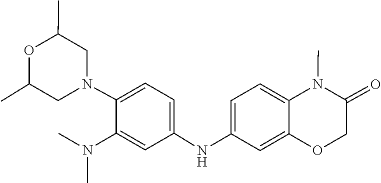 [SMILES: CC1CN(c2ccc(Nc3ccc4c(c3)OCC(=O)N4C)cc2N(C)C)CC(C)O1]

The title compound 20 (14.3 mg) was prepared in a total yield of 69.5% as a yellow solid from 6-(2,6-dimethylmorpholino)-N¹,N¹-dimethylbenzene-1,3-diamine (16.2 mg, 0.065 mmol), 1-(7-bromo-2,3-dihydro-4H-benzo[b][1,4]oxazin-4-yl)-2,2-dimethylpropan-1-one (14.9 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, DMSO-d₆) δ 6.98 (d, J=8.8 Hz, 1H), 6.81-6.66 (m, 2H), 6.64-6.56 (m, 3H), 4.57 (s, 2H), 3.85-3.65 (m, 2H), 3.34-3.29 (m, 2H), 3.22 (s, 3H), 2.76 (s, 6H), 2.18-2.09 (m, 2H), 1.10 (d, J=6.4 Hz, 6H). Mass (m/z): 411.3 [M+H]⁺.

### Compound 21

### 7-((4-(2-azaspiro[3.3]heptan-2-yl)phenyl)amino)-4-methyl-2H-benzo[b][1,4]oxazin-3(4H)-one

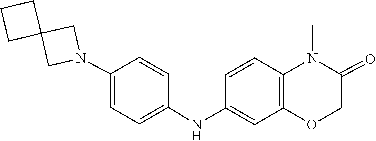 [SMILES: CN1C(=O)COc2cc(Nc3ccc(N4CC5(CCC5)C4)cc3)ccc21]

The title compound 21 (73.8 mg) was prepared in a total yield of 64.0% as a yellow solid from 4-(2-azaspiro[3.3]heptan-2-yl)aniline (81 mg, 0.43 mmol), 7-bromo-4-methyl-2H-benzo[b][1,4]oxazin-3(4H)-one (80 mg, 0.33 mmol), Pd₂(dba)₃ (3.0 mg, 3.3 umol), X-Phos (7.9 mg, 16.5 umol) and Cs₂CO₃ (163 mg, 0.50 mmol) in 1,4-dioxane (3.0 mL) according to the procedure for compound 1. ¹H NMR (400 MHz, Methanol-d₄) δ 7.07-6.88 (m, 2H), 6.61-6.47 (m, 2H), 4.54 (s, 2H), 3.34 (s, 3H), 2.24 (t, J=7.6 Hz, 4H), 1.96-1.88 (m, 2H), 1.41-1.19 (m, 4H). Mass (m/z): 350.2[M+H]⁺.

### Compound 22

### 7-((4-(diethylamino)phenyl)amino)-4-methyl-2H-benzo[b][1,4]oxazin-3(4H)-one

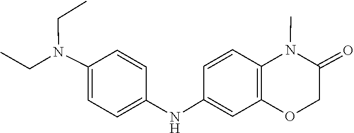 [SMILES: CCN(CC)c1ccc(Nc2ccc3c(c2)OCC(=O)N3C)cc1]

The title compound 22 (43.0 mg) was prepared in a total yield of 40.2% as a dark yellow solid from a mixture of N¹,N¹-diethylbenzene-1,4-diamine (70.5 mg, 0.43 mmol), 7-bromo-4-methyl-2H-benzo[b][1,4]oxazin-3(4H)-one (80 mg, 0.33 mmol), Pd₂(dba)₃ (3.0 mg, 3.3 umol), X-Phos (7.9 mg, 16.5 umol) and Cs₂CO₃ (163 mg, 0.50 mmol) in 1,4-dioxane (3.0 mL) according to the procedure for compound 1. ¹H NMR (400 MHz, Methanol-d₄) δ 8.26-5.99 (m, 5H), 4.51 (s, 2H), 3.92-3.63 (m, 4H), 3.34 (s, 3H), 1.10 (t, J=7.0 Hz, 6H). Mass (m/z): 326.2 [M+H]⁺.

### Compound 23

### 7-((4-(4-(fluoromethyl)piperidin-1-yl)phenyl)amino)-4-methyl-2H-benz o[b][1,4]oxazin-3(4H)-one

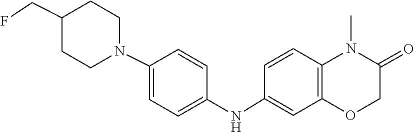 [SMILES: CN1C(=O)COc2cc(Nc3ccc(N4CCC(CF)CC4)cc3)ccc21]

The title compound 23 (63.3 mg) was prepared in a total yield of 82.9% as a brown solid from 4-(4-(fluoromethyl)piperidin-1-yl)aniline (52 mg, 0.248 mmol), 7-bromo-4-methyl-2H-benzo[b][1,4]oxazin-3(4H)-one (50 mg, 0.207 mmol), Pd₂(dba)₃ (2 mg, 0.002 mmol), X-Phos (6 mg, 0.01 mmol) and Cs₂CO₃ (102 mg, 0.310 mmol) and 1,4-dioxane (5 mL) according to the procedure for compound 1. ¹H NMR (400 MHz, DMSO-d₆) δ 7.86 (d, J=7.6 Hz, 1H), 6.96 (dd, J=8.8, 2.4 Hz, 3H), 6.91-6.86 (m, 2H), 6.62 (dd, J=8.8, 2.4 Hz, 1H), 6.54 (d, J=2.4 Hz, 1H), 4.56 (s, 2H), 4.39 (d, J=6.0 Hz, 1H), 4.27 (d, J=5.6 Hz, 1H), 3.56 (dt, J=12.8, 3.2 Hz, 2H), 3.22 (s, 3H), 2.58 (td, J=12.0, 2.4 Hz, 2H), 1.80-1.69 (m, 3H), 1.35 (qd, J=12.0, 4.0 Hz, 2H). Mass (m/z): 370.3 [M+H]⁺.

### Compound 24

### 4-ethyl-7-((6-(4-(trifluoromethyl)piperidin-1-yl)pyridin-3-yl)amino)-2H-benzo[b][1,4]oxazin-3(4H)-one

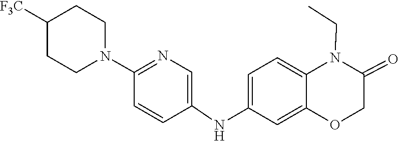 [SMILES: CCN1C(=O)COc2cc(Nc3ccc(N4CCC(C(F)(F)F)CC4)nc3)ccc21]

The title compound 24 (12.6 mg) was prepared in a total yield of 60.0% as a wheat solid from 6-(4-(trifluoromethyl)piperidin-1-yl)pyridin-3-amine (15.9 mg, 0.065 mmol), 7-bromo-4-ethyl-2H-benzo[b][1,4]oxazin-3(4H)-one (12.8 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, DMSO-d₆) δ 7.96 (d, J=2.8 Hz, 1H), 7.37 (dd, J=8.8, 2.8 Hz, 1H), 6.99 (d, J=8.8 Hz, 1H), 6.84 (d, J=8.8 Hz, 1H), 6.54 (dd, J=8.8, 2.8 Hz, 1H), 6.46 (d, J=2.8 Hz, 1H), 4.53 (s, 2H), 4.24-4.32 (m, 2H), 3.85 (q, J=7.2 Hz, 2H), 2.80-2.71 (m, 2H), 2.54 (m, 1H), 1.90-1.89 (m, 2H), 1.51-1.38 (m, 2H), 1.12 (t, J=7.2 Hz, 3H). Mass (m/z): 421.2 [M+H]⁺.

### Compound 25

### 4-ethyl-7-((6-(4-(trifluoromethyl)piperidin-1-yl)pyridin-3-yl)amino)-2H-benzo[b][1,4]oxazin-3(4H)-one

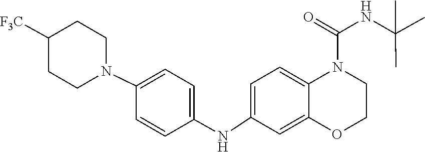 [SMILES: CC(C)(C)NC(=O)N1CCOc2cc(Nc3ccc(N4CCC(C(F)(F)F)CC4)cc3)ccc21]

The title compound 25 (15.4 mg) was prepared in a total yield of 64.6% as a yellow solid from 4-(4-(trifluoromethyl)piperidin-1-yl)aniline (15.9 mg, 0.065 mmol), 7-bromo-N-(tert-butyl)-2,3-dihydro-4H-benzo[b][1,4]oxazine-4-carboxamide (15.7 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, DMSO-d₆) δ 7.20 (d, J=8.8 Hz, 1H), 7.03-6.80 (m, 4H), 6.53-6.36 (m, 2H), 4.08 (t, J=4.4 Hz, 2H), 3.66-3.57 (m, 4H), 2.68-2.55 (m, 2H), 2.41 (m, 1H), 1.92-1.81 (m, 2H), 1.64-1.51 (m, 2H), 1.28 (s, 9H). Mass (m/z): 477.3 [M+H]⁺.

### Compound 26

### 4-methyl-7-((2-methyl-6-(4-(trifluoromethyl)piperidin-1-yl)pyridin-3-yl) amino)-2H-benzo[b][1,4]oxazin-3(4H)-one

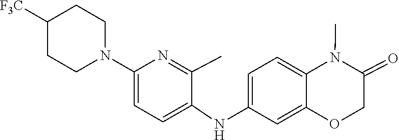 [SMILES: Cc1nc(N2CCC(C(F)(F)F)CC2)ccc1Nc1ccc2c(c1)OCC(=O)N2C]

The title Compound 26 (93.1 mg) was prepared in a total yield of 66.5% as a light yellow solid from 2-methyl-6-(4-(trifluoromethyl)piperidin-1-yl)pyridin-3-amine (111 mg, 0.43 mmol), 7-bromo-4-methyl-2H-benzo[b][1,4]oxazin-3(4H)-one (80 mg, 0.33 mmol), Pd₂(dba)₃ (3.0 mg, 3.3 umol), X-Phos (7.9 mg, 16.5 umol), and Cs₂CO₃ (163 mg, 0.50 mmol) according to the procedure for compound 1. ¹H NMR (400 MHz, DMSO-d₆) δ 7.31 (d, J=8.6 Hz, 1H), 7.26 (s, 1H), 6.92 (d, J=8.6 Hz, 1H), 6.71 (d, J=8.8 Hz, 1H), 6.27 (dd, J=8.6, 2.4 Hz, 1H), 6.22 (d, J=2.6 Hz, 1H), 4.54 (s, 2H), 4.40-4.32 (m, 2H), 3.21 (s, 3H), 2.83-2.73 (m, 2H), 2.60-2.53 (m, 1H), 2.23 (s, 3H), 1.91-1.84 (m, 1H), 1.44 (qd, J=12.6, 4.2 Hz, 2H). Mass (m/z): 421.3 [M+H]⁺.

### Compound 27

### 7-((5-fluoro-6-(4-(trifluoromethyl)piperidin-1-yl)pyridin-3-yl)amino)-4-methyl-2H-benzo[b][1,4]oxazin-3(4H)-one

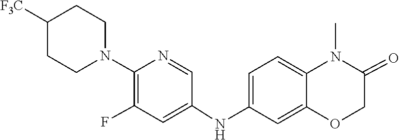 [SMILES: CN1C(=O)COc2cc(Nc3cnc(N4CCC(C(F)(F)F)CC4)c(F)c3)ccc21]

The title Compound 27 (62.2 mg) was prepared in a total yield of 44.4% as a Light yellow powder from 5-fluoro-6-(4-(trifluoromethyl)piperidin-1-yl) pyridin-3-amine (113 mg, 0.43 mmol), 7-bromo-4-methyl-2H-benzo[b][1,4]oxazin-3(4H)-one (80 mg, 0.33 mmol), Pd₂(dba)₃ (3.0 mg, 3.3 umol), X-Phos (7.9 mg, 16.5 umol), and Cs₂CO₃ (163 mg, 0.50 mmol) according to the procedure for compound 1. ¹H NMR (400 MHz, DMSO-d₆) δ 8.15 (s, 1H), 7.85 (dd, J=2.4, 1.0 Hz, 1H), 7.28 (dd, J=14.2, 2.3 Hz, 1H), 7.03 (d, J=8.8 Hz, 1H), 6.71 (dd, J=8.8, 2.5 Hz, 1H), 6.61 (d, J=2.5 Hz, 1H), 4.60 (s, 2H), 3.83-3.75 (m, 2H), 3.24 (s, 3H), 2.81 (td, J=12.7, 2.4 Hz, 2H), 2.53 (d, J=10.3 Hz, 1H), 1.92-1.84 (m, 2H), 1.57 (qd, J=12.6, 4.1 Hz, 2H). Mass (m/z): 425.2 [M+H]⁺.

### Compound 28

### 4-methyl-6-((4-(4-(trifluoromethyl)piperidin-1-yl)phenyl)amino)-2H-benzo[b][1,4]oxazin-3(4H)-one

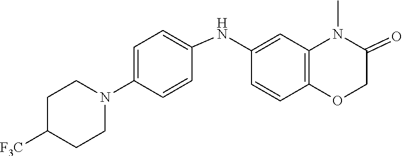 [SMILES: CN1C(=O)COc2ccc(Nc3ccc(N4CCC(C(F)(F)F)CC4)cc3)cc21]

The title compound 28 (39.4 mg) was prepared in a total yield of 47.0% as a brown solid from 4-(4-(trifluoromethyl)piperidin-1-yl)aniline (60 mg, 0.248 mmol), 6-bromo-4-methyl-2H-benzo[b][1,4]oxazin-3(4H)-one (50 mg, 0.207 mmol), Pd₂(dba)₃ (2 mg, 0.002 mmol), X-Phos (6 mg, 0.01 mmol) and Cs₂CO₃ (122 mg, 0.372 mmol) and 1,4-dioxane (5 mL) according to the procedure for compound 1. ¹H NMR (400 MHz, DMSO-d₆) δ 7.75 (s, 1H), 6.90 (d, J=8.8 Hz, 2H), 6.83-6.77 (m, 3H), 6.66 (d, J=2.4 Hz, 1H), 6.52 (dd, J=8.4, 2.4 Hz, 1H), 4.47 (s, 2H), 3.53 (d, J=12.0 Hz, 2H), 3.14 (s, 3H), 2.55 (t, J=11.6 Hz, 2H), 2.40-2.34 (m, 1H), 1.81 (d, J=12.4 Hz, 2H), 1.56-1.43 (m, 3H). Mass (m/z): 406.3 [M+H]⁺.

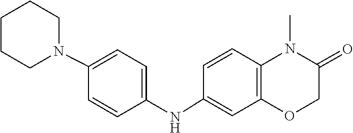 [SMILES: CN1C(=O)COc2cc(Nc3ccc(N4CCCCC4)cc3)ccc21]

The title compound 29 (64.9 mg) was prepared in a total yield of 93.0% as a brown solid from 4-(piperidin-1-yl)aniline (43 mg, 0.248 mmol), 6-bromo-4-methyl-2H-benzo[b][1,4]oxazin-3(4H)-one (50 mg, 0.207 mmol), Pd₂(dba)₃ (2 mg, 0.002 mmol), X-Phos (6 mg, 0.01 mmol) and Cs₂CO₃ (122 mg, 0.372 mmol) and 1,4-dioxane (5 mL) according to the procedure for compound 1. ¹H NMR (400 MHz, DMSO-d₆) δ 7.80 (s, 1H), 6.96 (dd, J=8.8, 6.4 Hz, 3H), 6.89-6.84 (m, 2H), 6.61 (dd, J=8.6, 2.5 Hz, 1H), 6.53 (d, J=2.5 Hz, 1H), 4.56 (s, 2H), 3.22 (s, 3H), 3.04-2.98 (m, 4H), 1.62 (q, J=4.1, 2.4 Hz, 4H), 1.51 (q, J=6.8, 6.4 Hz, 2H). Mass (m/z): 338.3 [M+H]⁺.

### Compound 30

### 7-((4-(ethyl(pentyl)amino)phenyl)amino)-4-methyl-2H-benzo[b][1,4]oxazin-3(4H)-one

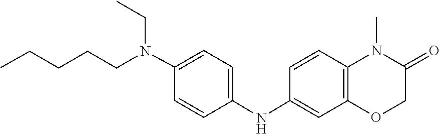 [SMILES: CCCCCN(CC)c1ccc(Nc2ccc3c(c2)OCC(=O)N3C)cc1]

The title compound 30 (33.1 mg) was prepared in a total yield of 43.5% as a white solid from N¹-ethyl-N¹-pentylbenzene-1,4-diamine (51 mg, 0.248 mmol), 6-bromo-4-methyl-2H-benzo[b][1,4]oxazin-3(4H)-one (50 mg, 0.207 mmol), Pd₂(dba)₃ (2 mg, 0.002 mmol), X-Phos (6 mg, 0.01 mmol) and Cs₂CO₃ (122 mg, 0.372 mmol) and 1,4-dioxane (5 mL) according to the procedure for compound 1. ¹H NMR (400 MHz, DMSO-d₆) δ 7.64 (s, 1H), 6.93 (d, J=8.4 Hz, 3H), 6.62 (d, J=8.4 Hz, 2H), 6.53 (d, J=8.8 Hz, 1H), 6.45 (d, J=2.4 Hz, 1H), 4.54 (s, 2H), 3.28 (d, J=7.2 Hz, 2H), 3.21 (s, 3H), 3.16 (d, J=7.6 Hz, 2H), 1.54-1.46 (m, 2H), 1.29 (td, J=12.0, 10.0, 5.6 Hz, 4H), 1.05 (t, J=6.8 Hz, 3H), 0.88 (t, J=6.8 Hz, 3H). Mass (m/z): 368.3 [M+H]⁺.

### Compound 31

### 7-((4-(ethyl(3-methoxypropyl)amino)phenyl)amino)-4-methyl-2H-benzo[b][1,4]oxazin-3(4H)-one

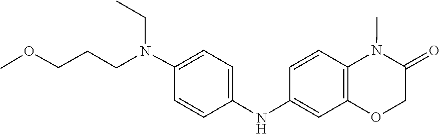 [SMILES: CCN(CCCOC)c1ccc(Nc2ccc3c(c2)OCC(=O)N3C)cc1]

The title compound 31 (53.6 mg) was prepared in a total yield of 70.2% as a black oil from N‘-ethyl-N’-(3-methoxypropyl)benzene-1,4-diamine (51 mg, 0.248 mmol), 6-bromo-4-methyl-2H-benzo[b][1,4]oxazin-3(4H)-one (50 mg, 0.207 mmol), Pd₂(dba)₃ (2 mg, 0.002 mmol), X-Phos (6 mg, 0.01 mmol) and Cs₂CO₃ (122 mg, 0.372 mmol) and 1,4-dioxane (5 mL) according to the procedure for compound 1. ¹H NMR (400 MHz, DMSO-d₆) δ 7.66 (s, 1H), 6.93 (d, J=8.4 Hz, 3H), 6.67-6.62 (m, 2H), 6.54 (dd, J=8.8, 2.4 Hz, 1H), 6.46 (d, J=2.4 Hz, 1H), 4.54 (s, 2H), 3.37 (s, 1H), 3.36-3.34 (m, 2H), 3.31-3.25 (m, 3H), 3.24 (s, 3H), 3.21 (s, 3H), 1.77-1.66 (m, 2H), 1.05 (t, J=6.8 Hz, 3H). Mass (m/z): 370.3 [M+H]⁺.

### Compound 32

### 7-((3-methoxy-4-(pentyloxy)phenyl)amino)-4-methyl-2H-benzo[b][1,4]oxazin-3(4H)-one

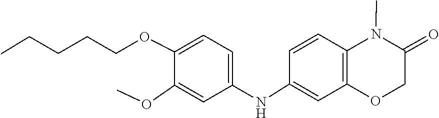 [SMILES: CCCCCOc1ccc(Nc2ccc3c(c2)OCC(=O)N3C)cc1OC]

The title compound 32 (38.8 mg) was prepared in a total yield of 50.7% as a pink solid from 3-methoxy-4-(pentyloxy)aniline (52 mg, 0.248 mmol), 6-bromo-4-methyl-2H-benzo[b][1,4]oxazin-3(4H)-one (50 mg, 0.207 mmol), Pd₂(dba)₃ (2 mg, 0.002 mmol), X-Phos (6 mg, 0.01 mmol) and Cs₂CO₃ (122 mg, 0.372 mmol) and 1,4-dioxane (5 mL) according to the procedure for compound 1. ¹H NMR (400 MHz, DMSO-d₆) δ 7.93 (s, 1H), 6.99 (d, J=8.8 Hz, 1H), 6.84 (d, J=8.8 Hz, 1H), 6.71-6.64 (m, 2H), 6.61-6.54 (m, 2H), 4.57 (s, 2H), 3.87 (t, J=6.4 Hz, 2H), 3.71 (s, 3H), 3.23 (s, 3H), 1.68 (p, J=6.8 Hz, 2H), 1.43-1.28 (m, 4H), 0.90 (t, J=7.2 Hz, 3H). Mass (m/z): 371.3 [M+H]⁺.

### Compound 33

### 7-((4-fluoro-3-(4-(trifluoromethyl)piperidin-1-yl)phenyl)amino)-4-methyl-2H-benzo[b][1,4]oxazin-3(4H)-one

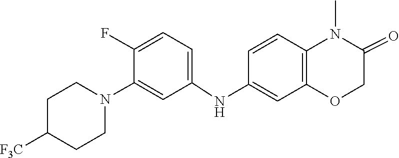 [SMILES: CN1C(=O)COc2cc(Nc3ccc(F)c(N4CCC(C(F)(F)F)CC4)c3)ccc21]

The title compound 33 (16.9 mg) was prepared in a total yield of 15.9% as a light pink solid from 4-fluoro-3-(4-(trifluoromethyl) piperidin-1-yl) aniline (86 mg, 0.33 mmol), 7-bromo-4-methyl-2H-benzo[b][1,4]oxazin-3(4H)-one (60 mg, 0.25 mmol), Pd₂(dba)₃ (2.3 mg, 2.5 umol), X-Phos (6.0 mg, 12.5 umol), and Cs₂CO₃ (122 mg, 0.75 mmol) according to the procedure for compound 1. ¹H NMR (300 MHz, DMSO-d₆) δ 8.03 (s, 1H), 7.03-6.95 (m, 2H), 6.72 (dd, J=8.6, 2.5 Hz, 1H), 6.68-6.58 (m, 3H), 4.59 (s, 2H), 3.36-3.29 (m, 4H), 3.24 (s, 3H), 2.70-2.62 (m, 2H), 2.48-2.41 (m, 1H), 1.94-1.86 (m, 2H), 1.61 (qd, J=12.4, 4.1 Hz, 2H). Mass (m/z): 424.2 [M+H]⁺.

### Compound 34

### 7-((2-fluoro-4-(4-(trifluoromethyl)piperidin-1-yl)phenyl)amino)-4-methyl-2H-benzo[b][1,4]oxazin-3(4H)-one

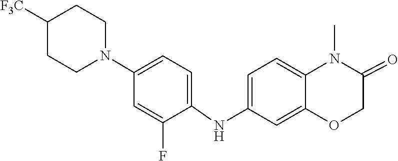 [SMILES: CN1C(=O)COc2cc(Nc3ccc(N4CCC(C(F)(F)F)CC4)cc3F)ccc21]

The title compound 34 (38.1 mg) as prepared in a total yield of 28.2% as a light gray solid from 2-fluoro-4-(4-(trifluoromethyl)piperidin-1-yl)aniline (100 mg, 0.38 mmol), 7-bromo-4-methyl-2H-benzo[b][1,4] oxazin-3(4H)-one (76 mg, 0.32 mmol), Pd₂(dba)₃ (2.9 mg, 3.2 umol), X-Phos (7.6 mg, 16 umol), and Cs₂CO₃ (156 mg, 0.48 mmol) according to the procedure for compound 1. ¹H NMR (400 MHz, DMSO-d₆) δ 7.54 (s, 1H), 7.14-7.07 (m, 1H), 6.95 (d, J=8.7 Hz, 1H), 6.87 (dd, J=14.3, 2.7 Hz, 1H), 6.74 (dd, J=8.7, 2.7 Hz, 1H), 6.46 (dd, J=8.6, 2.5 Hz, 1H), 6.37 (d, J=2.5 Hz, 1H), 4.55 (s, 2H), 3.78-3.68 (m, 2H), 3.21 (s, 3H), 2.69 (td, J=12.5, 2.6 Hz, 2H), 2.49-2.40 (m, 1H), 1.90-1.83 (m, 2H), 1.54 (qd, J=12.6, 4.1 Hz, 2H). Mass (m/z): 424.3 [M+H]⁺.

### Compound 35

### 7-((4-(4,4-dimethylcyclohexyl)phenyl)amino)-4-methyl-2H-benzo[b][1,4]oxazin-3(4H)-one

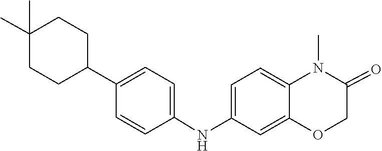 [SMILES: CN1C(=O)COc2cc(Nc3ccc(C4CCC(C)(C)CC4)cc3)ccc21]

The title compound 35 (15.9 mg) was prepared in a total yield of 87.10% as a yellow powder from 4-(4,4-dimethylcyclohexyl)aniline (13.2 mg, 0.065 mmol), 7-bromo-4-methyl-2H-benzo[b][1,4]oxazin-3(4H)-one (12.1 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (300 MHz, DMSO-d₆) δ 7.14-7.03 (m, 2H), 7.02-6.90 (m, 3H), 6.72 (dd, J=8.8, 2.4 Hz, 1H), 6.63 (d, J=2.4 Hz, 1H), 4.57 (s, 2H), 3.23 (s, 3H), 2.31 (m, 1H), 1.63-1.49 (m, 4H), 1.49-1.37 (m, 2H), 1.35-1.22 (m, 2H), 0.95 (s, 3H), 0.93 (s, 3H). Mass (m/z): 365.2 [M+H]⁺.

### Compound 36

### 7-((2-(4-isopropylpiperidin-1-yl)pyrimidin-5-yl)amino)-4-methyl-2H-benzo[b][1,4]oxazin-3(4H)-one

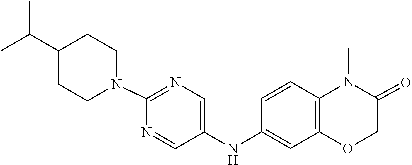 [SMILES: CC(C)C1CCN(c2ncc(Nc3ccc4c(c3)OCC(=O)N4C)cn2)CC1]

The title compound 36 (62.1 mg) was prepared in a total yield of 78.6% as a yellow solid from 2-(4-isopropylpiperidin-1-yl)pyrimidin-5-amine (55 mg, 0.248 mmol), 6-bromo-4-methyl-2H-benzo[b][1,4]oxazin-3(4H)-one (50 mg, 0.207 mmol), Pd₂(dba)₃ (2 mg, 0.002 mmol), X-Phos (6 mg, 0.01 mmol), Cs₂CO₃ (122 mg, 0.372 mmol) and 1,4-dioxane (5 mL) according to the procedure for compound 1. ¹H NMR (400 MHz, DMSO-d₆) δ 8.17 (s, 2H), 7.67 (d, J=4.4 Hz, 1H), 6.91 (d, J=8.8 Hz, 1H), 6.42 (dd, J=8.8, 2.4 Hz, 1H), 6.35 (d, J=2.4 Hz, 1H), 4.60 (dt, J=12.8, 2.4 Hz, 2H), 4.52 (s, 2H), 3.17 (s, 3H), 2.71 (td, J=12.8, 2.4 Hz, 2H), 1.65 (dd, J=12.4, 2.8 Hz, 2H), 1.39 (dq, J=13.2, 6.8 Hz, 1H), 1.29-1.00 (m, 4H), 0.83 (d, J=6.8 Hz, 6H). Mass (m/z): 382.3 [M+H]⁺.

### Compound 37

### 7-((5,5-dimethyl-5,6,7,8-tetrahydronaphthalen-2-yl)amino)-4-methyl-2H-benzo[b][1,4]oxazin-3(4H)-one

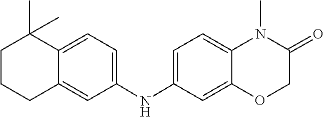 [SMILES: CN1C(=O)COc2cc(Nc3ccc4c(c3)CCCC4(C)C)ccc21]

The title compound 37 (125.0 mg) was prepared in a total yield of 88.1% as a yellow solid from 6-bromo-1,1-dimethyl-1,2,3,4-tetrahydronaphthalene (0.1 g, 0.42 mmol), 7-amino-4-methyl-2H-benzo[b][1,4]oxazin-3(4H)-one (0.09 g, 0.5 mmol), Ruphos (39 mg, 0.08 mmol), Pd₂(dba)₃ (38 mg, 0.04 mmol) and Cs₂CO₃ (0.41 g, 1.26 mmol) in 1,4-dioxane (10 mL) according to the procedure for compound 1. ¹H NMR (400 MHz, CDCl₃) δ 7.23 (d, J=8.4 Hz, 1H), 6.88-6.83 (m, 2H), 6.73 (d, J=13.6 Hz, 3H), 4.59 (s, 2H), 3.49 (s, 1H), 3.34 (s, 3H), 2.71 (t, J=6.4 Hz, 2H), 1.81-1.76 (m, 2H), 1.66-1.64 (m, 2H), 1.27 (s, 6H). Mass (m/z): 337.2 [M+H]⁺.

### Compound 38

### 4-methyl-7-((4-(tert-pentyl)phenyl)amino)-2H-benzo[b][1,4]oxazin-3(4H)-one

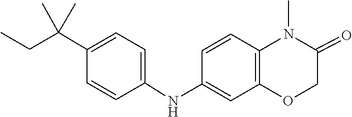 [SMILES: CCC(C)(C)c1ccc(Nc2ccc3c(c2)OCC(=O)N3C)cc1]

The title compound 38 (60.0 mg) was prepared in a total yield of 33.2% as a white solid from 4-(tert-pentyl)aniline (100 mg, 0.613 mmol), 7-bromo-4-methyl-2H-benzo[b][1,4]oxazin-3(4H)-one (134 mg, 0.558 mmol), Pd₂(dba)₃ (25.5 mg, 0.028 mmol), X-Phos (26.6 mg, 0.056 mmol) and Cs₂CO₃ (364 mg, 1.12 mmol) in dioxane (15 mL) according to the procedure for compound 1. ¹H NMR (400 MHz, CDCl₃) δ 7.41-7.27 (m, 4H), 7.12 (t, J=10.6 Hz, 2H), 6.59 (t, J=8.6 Hz, 2H), 4.36-4.23 (m, 2H), 3.85 (d, J=15.9 Hz, 2H), 1.64-1.54 (m, 3H), 1.24 (d, J=10.9 Hz, 7H), 0.68 (t, J=7.4 Hz, 3H). Mass (m/z): 325.4 [M+H]⁺.

### Compound 39

### 4-methyl-7-((1,1,3,3-tetramethyl-2,3-dihydro-1H-inden-5-yl)amino)-2H-benzo[b][1,4]oxazin-3(4H)-one

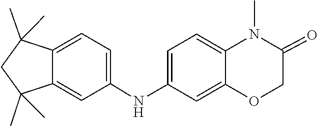 [SMILES: CN1C(=O)COc2cc(Nc3ccc4c(c3)C(C)(C)CC4(C)C)ccc21]

The title compound 39 (8 mg) was prepared as a yellow solid from 5-bromo-1,1,3,3-tetramethyl-2,3-dihydro-1H-indene (300 mg, 1.19 mmol), 7-amino-4-methyl-2H-benzo[b][1,4]oxazin-3(4H)-one (211 mg, 1.19 mmol), Cs₂CO₃ (772 mg, 2.38 mmol), Pd₂(dba)₃ (108 mg, 0.12 mmol) and Ruphos (110 mg, 0.24 mmol) in 1,4-dioxane (15 mL) according to the procedure for compound 1. ¹H NMR (400 MHz, CD₃OD) δ 6.97 (dd, J=8.4, 3.2 Hz, 2H), 6.88 (dd, J=8.2, 2.2 Hz, 1H), 6.79 (d, J=2.0 Hz, 1H), 6.70 (dd, J=8.6, 2.6 Hz, 1H), 6.64 (d, J=2.6 Hz, 1H), 4.53 (s, 2H), 3.30 (s, 3H), 1.89 (s, 2H), 1.26 (d, J=1.8 Hz, 12H). Mass (m/z): 351.3 [M+H]⁺.

### Compound 40

### 1-(2-(3-oxo-7-((4-(4-(trifluoromethyl)piperidin-1-yl)phenyl)amino)-2,3-dihydro-4H-benzo[b][1,4]oxazin-4-yl)ethyl)urea

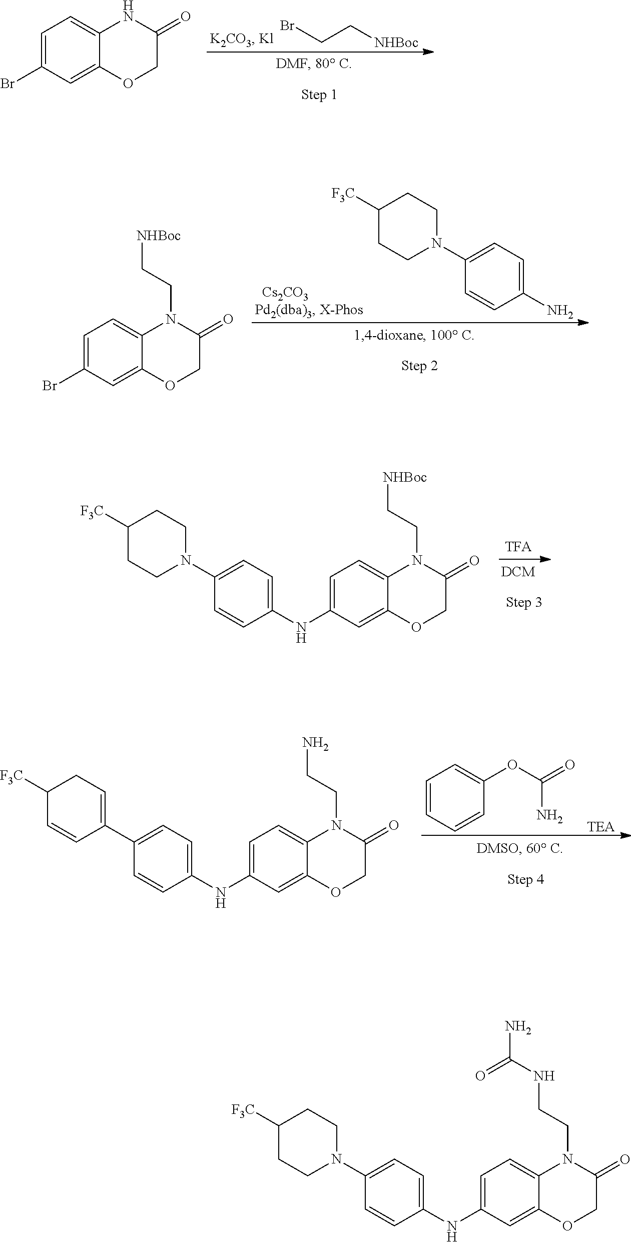

### Step 1. Preparation of tert-butyl (2-(7-bromo-3-oxo-2,3-dihydro-4H-benzo[b][1,4]oxazin-4-yl)ethyl)carbamate

A mixture of 7-bromo-2H-1,4-benzoxazine-3(4H)-one (454 mg, 2.0 mmol), tert-Butyl N-(2-bromoethyl)carbamate (892 mg, 4.0 mmol), KI (33.2 mg, 0.2 mmol) and K₂CO₃ (828 mg, 6.0 mmol) in DMF (10.0 mL) was stirred for 18 hours at 80° C. After cooling to r.t., 10 mL of water was added. The resulting solution was extracted with 3×10 mL of ethyl acetate. The organic layers were combined, washed with water (3×15 mL), dried and concentrated under vacuum. The residue was purified by a silica gel column (EA/PE=0-1/2) to afford the desired product as a yellow solid (250 mg, 33.8%). Mass (m/z): 3 71.2 [M+H]⁺.

### Step 2. Preparation of tert-butyl (2-(3-oxo-7-((4-(4-(trifluoromethyl) piperidin-1-yl)phenyl)amino)-2,3-dihydro-4H-benzo[b][1,4]oxazin-4-yl)et hyl)carbamate

The title compound (220 mg) as prepared in a total yield of 60.6% as a light yellow solid from 4-(4-(trifluoromethyl)piperidin-1-yl)aniline (214 mg, 0.89 mmol), tert-butyl (2-(7-bromo-3-oxo-2,3-dihydro-4H-benzo[b][1,4] oxazin-4-yl)ethyl)carbamate (250 mg, 0.68 mmol), Pd₂(dba)₃ (5.0 mg, 6.8 umol), X-Phos (16.3 mg, 34 umol), and Cs₂CO₃ (333 mg, 1.02 mmol) according to the procedure for compound 1. Mass (m/z): 535.2 [M+H]⁺.

### Step 3. Preparation of 4-(2-aminoethyl)-7-((4′-(trifluoromethyl)-3′,4′-dihydro-[1,1′-biphenyl]-4-yl)amino)-2H-benzo[b][1,4]oxazin-3(4H)-one

To a solution of tert-butyl (2-(3-oxo-7-((4-(4-(trifluoromethyl)piperidin-1-yl)phenyl)amino)-2,3-dihydro-4H-benzo[b][1,4]oxazin-4-yl)ethyl)carba mate (220 mg, 0.41 mmol) in DCM (5 mL) was added TFA (5 mL). Then the solution was stirred for 30 mins at r.t. and concentrated. 10 ml water was added. The pH of the filtration was adjusted to 8-9 with sodium carbonate solution. Then the mixture was extracted by DCM (15 mL×3). The combined organic layers were washed with water (10 mL), dried over Na₂SO₄ and concentrated. The residue was purified by perp-TLC (MeOH/DCM=1/5) to afford the desired product as a yellow solid. Mass (m/z): 435.1 [M+H]⁺.

### Step 4. Preparation of 1-(2-(3-oxo-7-((4-(4-(trifluoromethyl)piperidin-1-yl)phenyl)amino)-2,3-dihydro-4H-benzo[b][1,4]oxazin-4-yl)ethyl)urea (40)

To a solution of 4-(2-aminoethyl)-7-((4′-(trifluoromethyl)-3′,4′-dihydro-[1,1′-biphenyl]-4-yl)amino)-2H-benzo[b][1,4]oxazin-3(4H)-one (160 mg, 0.37 mmol) and TEA (112 mg, 1.11 mmol) in DMSO (3.0 mL) was added phenyl carbamate (60.6 mg, 0.44 mmol). Then the mixture was stirred overnight at 60° C. After cooling to rt., 10 mL of water was added. The resulting solution was extracted with 3×10 mL of ethyl acetate. The organic layers were combined, washed with water (3×15 mL), dried and concentrated under vacuum. The residue was purified by prep-TLC (MeOH/DCM=1/10) to afford the desired product (compound 40) as a yellow solid (17.5 mg, 9.9%). ¹H NMR (400 MHz, DMSO-d₆) δ 7.81 (s, 1H), 7.18 (d, J=8.8 Hz, 1H), 6.99-6.94 (m, 2H), 6.93-6.87 (m, 2H), 6.60 (dd, J=8.7, 2.5 Hz, 1H), 6.54 (d, J=2.4 Hz, 1H), 6.14 (t, J=5.9 Hz, 1H), 5.52 (s, 2H), 4.54 (s, 2H), 3.81 (t, J=7.0 Hz, 2H), 3.67-3.57 (m, 2H), 3.15 (q, J=6.5 Hz, 2H), 2.66-2.58 (m, 2H), 2.46-2.36 (m, 1H), 1.94-1.84 (m, 2H), 1.57 (qd, J=12.5, 4.1 Hz, 2H). Mass (m/z): 478.2 [M+H]⁺.

### Compound 41

### 4-(3-(methylamino)propyl)-7-((4-(4-(trifluoromethyl)piperidin-1-yl) phenyl)amino)-2H-benzo[b][1,4]oxazin-3(4H)-one

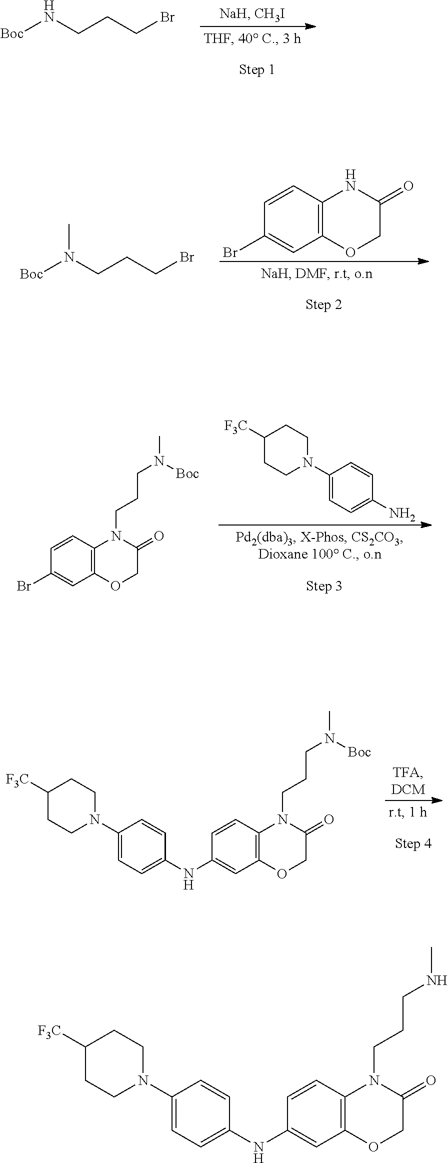

### Step 1. Preparation of tert-butyl (3-bromopropyl)(methyl)carbamate

To a solution of tert-butyl (3-bromopropyl)carbamate (1.00 g, 4.20 mmol) in THF (10 mL) was added NaH (60%, 252 mg, 6.30 mmol) in portions at 0° C. The mixture was stirred at r.t. for 1 h. CH₃I was then added to the above mixture and stirred at 35° C. for 12 h. The reaction mixture was quenched by the addition of water (20 mL) and extracted with ethyl acetate (3×50 mL). The combined organic phase was dried and concentrated to give the titled compound (1.06 g, theoretical yield) as a light yellow oil. LC-MS (m/z) 252.2 [M+H]⁺.

### Step 2. Preparation of tert-butyl(3-(7-bromo-3-oxo-2,3-dihydro-4H-benzo[b][1,4]oxazin-4-yl)propyl)(methyl)carbamate

To a solution of 7-bromo-2H-benzo[b][1,4]oxazin-3(4H)-one (200 mg, 0.88 mmol) in DMF (3 mL) was added NaH (60%, 52.6 mg, 1.31 mmol) in portions at 0° C. The mixture was stirred at r.t. for 1 h. Tert-butyl (3-bromopropyl)(methyl)carbamate (221.13 mg, 0.877 mmol) was then added to the above mixture and stirred at room temperature for 12 h. The reaction mixture was quenched by the addition of water (20 mL) and extracted with ethyl acetate (3×50 mL). The combined organic phase was dried and concentrated. The residue was purified by TLC (Petroleum ether:Ethyl acetate=3:1) to give the titled compound (340 mg, 97% yield) as a light yellow oil. LC-MS (m/z) 399.2 [M+H]⁺.

### Step 3. Preparation of tert-butylmethyl(3-(3-oxo-7-((4-(4-(trifluoromethyl) piperidin-1-yl)phenyl)amino)-2,3-dihydro-4H-benzo[b][1,4]oxazin-4-yl)pr opyl)carbamate

The title compound (60 mg) as prepared in a total yield of 42.6% as light yellow oil from tert-butyl (3-(7-bromo-3-oxo-2,3-dihydro-4H-benzo[b][1,4] oxazin-4-yl)propyl)(methyl)carbamate (100 mg, 0.25 mmol) and 4-(4-(trifluoromethyl)piperidin-1-yl)aniline (67.3 mg, 0.27 mmol), Pd₂(dba)₃ (11.5 mg, 0.013 mmol), X-phos (11 mg, 0.025 mmol) and Cs₂CO₃ (163 mg, 0.5 mmol) according to the procedure for compound 1. LC-MS (m/z) 563.2 [M+H]⁺.

### Step 4. 4-(3-(methylamino)propyl)-7-((4-(4-(trifluoromethyl)piperidin-1-yl)phenyl)amino)-2H-benzo[b][1,4]oxazin-3(4H)-one (41)

To a solution of 4-(3-(methylamino)propyl)-7-((4-(4-(trifluoromethyl) piperidin-1-yl)phenyl)amino)-2H-benzo[b][1,4]oxazin-3(4H)-one (60 mg, 0.11 mmol) in DCM (3 mL) was added TFA (1 mL). The mixture was stirred at r.t. for 1 h. The reaction mixture was concentrated under vacuum. The residue was quenched by the addition of a saturated solution of NaHCO₃ (10 mL) and extracted with ethyl acetate (3×20 mL). The combined organic phase was dried and concentrated. The residue was purified by Prep TLC (Dichloromethane:Methanol:Ammonia=5:1:0.1) to give the titled compound 41 (18.7 mg, 37.9% yield) as a light green solid. ¹H NMR (400 MHz, DMSO-d₆): δ 8.82 (s, 2H), 7.88 (s, 111), 7.09 (d, J=8.7 Hz, 111), 6.97 (d, J=8.2 Hz, 2H), 6.91 (s, 2H), 6.63 (d, J=8.7 Hz, 111), 6.57 (s, 111), 4.58 (s, 2H), 3.94-3.90 (t, J=7.2 Hz, 2H), 3.62 (d, J=12.1 Hz, 2H), 2.93 (d, J=8.5 Hz, 2H), 2.68-2.58 (m, 2H), 2.47-2.39 (m, 2H), 2.53 (s, 1H), 1.94-1.84 (m, 4H), 1.58 (s, 2H). LC-MS (m/z) 463.2 [M+H]⁺.

### Compound 42

### 7-((1,1-dimethyl-2,3-dihydro-1H-inden-5-yl)amino)-4-methyl-2H-benzo[b][1,4]o xazin-3(4H)-one

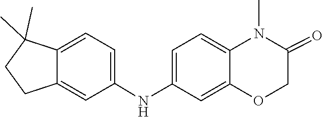 [SMILES: CN1C(=O)COc2cc(Nc3ccc4c(c3)CCC4(C)C)ccc21]

The title compound (16 mg) was prepared in a total yield of 8% as a yellow solid from 1,1-dimethyl-2,3-dihydro-1H-inden-5-amine (0.1 g, 0.6 mmol), 7-bromo-4-methyl-2H-benzo[b][1,4]oxazin-3(4H)-one (0.15 g, 0.6 mmol), Xantphos (69 mg, 0.12 mmol), Cs₂CO₃ (0.39 g, 1.2 mmol), Pd₂(dba)₃ (50 mg, 0.06 mmol) and toluene (10 mL) according to the procedure for compound 1. MS (ESI) m/z 322.8 [M+H]. ¹H NMR (300 MHz, CD₃OD) δ 7.11-6.80 (m, 4H), 6.72-6.62 (m, 2H), 4.55 (s, 2H), 3.32-3.27 (m, 3H), 2.80 (q, J=6 Hz, 2H), 1.91 (q, J=6 Hz, 2H), 1.23 (s, 6H).

### Compound 43

### 4-(2-(methylamino)ethyl)-7-((4-(4-(trifluoromethyl)piperidin-1-yl)phenyl) amino)-2H-benzo[b][1,4]oxazin-3(4H)-one

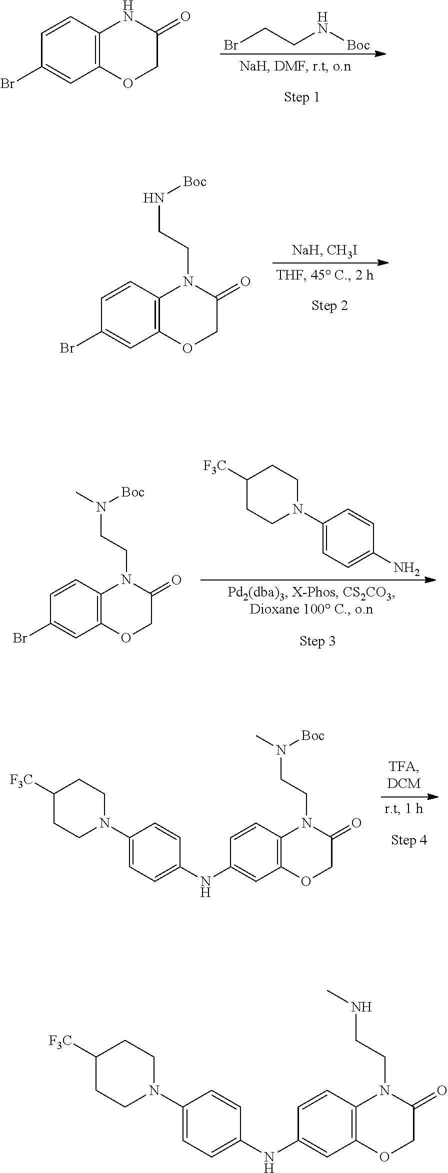

### Step 1. Preparation of tert-butyl(2-(7-bromo-3-oxo-2,3-dihydro-4H-benzo[b][1,4]oxazin-4-yl)ethyl)carbamate

To a solution of 7-bromo-2H-benzo[b][1,4]oxazin-3(4H)-one (150 mg, 0.66 mmol) in DMF (10 mL) was added NaH (60%, 162 mg, 0.72 mmol) in portions at 0° C. The mixture was stirred at r.t. for 1 h. Tert-butyl (2-bromoethyl)carbamate (31.6 mg, 0.79 mmol) was then added to the above mixture and stirred at room temperature for 12 h. The reaction mixture was quenched by the addition of water (20 mL) and extracted with ethyl acetate (3×50 mL). The combined organic phase was dried and concentrated. The residue was purified by Prep-TLC (petroleum ether:ethyl acetate=1:1) to afford the titled compound (236 mg, 96.6% yield) as a light yellow oil. LC-MS (m/z) 371.2 [M+H]⁺.

### Step 2. Preparation of tert-butyl(2-(7-bromo-3-oxo-2,3-dihydro-4H-benzo[b][1,4]oxazin-4-yl)ethyl)(methyl)carbamate

To a solution of tert-butyl (2-(7-bromo-3-oxo-2,3-dihydro-4H-benzo[b][1,4] oxazin-4-yl)ethyl)carbamate (236 mg, 0.64 mmol) in THE (3 mL) was added NaH (60%, 30.5 mg, 0.76 mmol) in portions at 0° C. The mixture was stirred at r.t. for 1 h. CH₃I (108.33 mg, 0.76 mmol) was then added to the above mixture and stirred at room temperature for 2 h. The reaction mixture was quenched by the addition of water (20 mL) and extracted with ethyl acetate (3×50 mL). The combined organic phase was dried and concentrated to give the titled compound (228 mg, 93.1% yield) as brown oil. LC-MS (m/z) 385.2 [M+H]⁺.

### Step 3. Preparation of tert-butylmethyl(2-(3-oxo-7-((4-(4-(trifluoromethyl) piperidin-1-yl)phenyl)amino)-2,3-dihydro-4H-benzo[b][1,4]oxazin-4-yl)et hyl)carbamate

The title compound (70 mg) was prepared in a total yield of 42.7% as light yellow oil from tert-butyl (2-(7-bromo-3-oxo-2,3-dihydro-4H-benzo[b][1,4]oxazin-4-yl)ethyl)(methyl)carbamate (115 mg, 0.30 mmol), 4-(4-(trifluoromethyl)piperidin-1-yl)aniline (80.2 mg, 0.33 mmol), Pd₂(dba)₃ (13.7 mg, 0.015 mmol), X-phos (14.2 mg, 0.03 mmol) and Cs₂CO₃ (195 mg, 0.60 mmol) in 1,4-dioxane (5 mL) according to the procedure for compound 1. LC-MS (m/z) 549.2 [M+H]⁺.

### Step 4. Preparation of 4-(2-(methylamino)ethyl)-7-((4-(4-(trifluoromethyl) piperidin-1-yl)phenyl)amino)-2H-benzo[b][1,4]oxazin-3(4H)-one

The title compound 43 (20.1 mg) was prepared in a total yield of 35.1% as a khaki solid from methyl(2-(3-oxo-7-((4-(4-(trifluoromethyl) piperidin-1-yl)phenyl)amino)-2,3-dihydro-4H-benzo[b][1,4]oxazin-4-yl)e thyl)carbamate (70 mg, 0.13 mmol), TFA (1 mL) and DCM (3 mL) according to the procedure for compound 41. ¹H NMR (400 MHz, DMSO-d₆): δ 7.82 (s, 1H), 7.05 (d, J=8.8 Hz, 1H), 6.96-6.89 (m, 2H), 6.91-6.83 (m, 2H), 6.61-6.49 (m, 2H), 4.55 (s, 2H), 4.05 (t, J=6.4 Hz, 2H), 3.59 (d, J=12.3 Hz, 2H), 3.26 (s, 3H), 2.96 (t, J=6.4 Hz, 2H), 2.59 (td, J=12.3, 2.5 Hz, 2H), 2.45-2.35 (m, 2H), 1.90-1.81 (m, 2H), 1.53 (qd, J=12.4, 4.1 Hz, 2H). LC-MS (m/z) 449.2 [M+H]⁺.

### Compound 44

### 7-((4-(2,6-dimethylmorpholino)phenyl)amino)-4-methyl-2H-benzo[b][1,4]oxazin-3(4H)-one

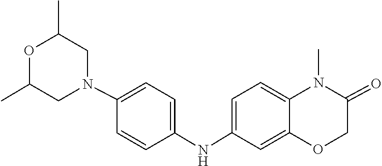 [SMILES: CC1CN(c2ccc(Nc3ccc4c(c3)OCC(=O)N4C)cc2)CC(C)O1]

The title compound 44 (68.5 mg) was prepared in a total yield of 34.7% as a khaki solid from 7-bromo-4-methyl-2H-benzo[b][1,4]oxazin-3(4H)-one (130 mg, 0.54 mmol), 4-(2,6-dimethylmorpholino)aniline (122 mg, 0.59 mmol), Pd₂(dba)₃ (24.6 mg, 0.03 mmol), X-Phos (25.6 mg, 0.054 mmol) and Cs₂CO₃ (350 mg, 1.07 mmol) in 1,4-dioxane (5 mL) according to the procedure for compound 1. LC-MS (m/z) 368.2 [M+H]⁺. ¹H NMR (400 MHz, DMSO-d₆): δ 7.75 (s, 1H), 6.96-6.87 (m, 3H), 6.87-6.80 (m, 2H), 6.58 (dd, J=8.7, 2.5 Hz, 1H), 6.49 (d, J=2.5 Hz, 1H), 4.53 (s, 2H), 3.69-3.61 (m, 2H), 3.40 (dt, J=11.1, 2.3 Hz, 2H), 3.18 (s, 3H), 2.15 (t, J=11.2 Hz, 2H), 1.10 (d, J=6.2 Hz, 6H).

### Compound 45

### 7-((4-(2,6-dimethylmorpholino)phenyl)amino)-4-(3-(methylamino) propyl)-2H-benzo[b][1,4]oxazin-3(4H)-one

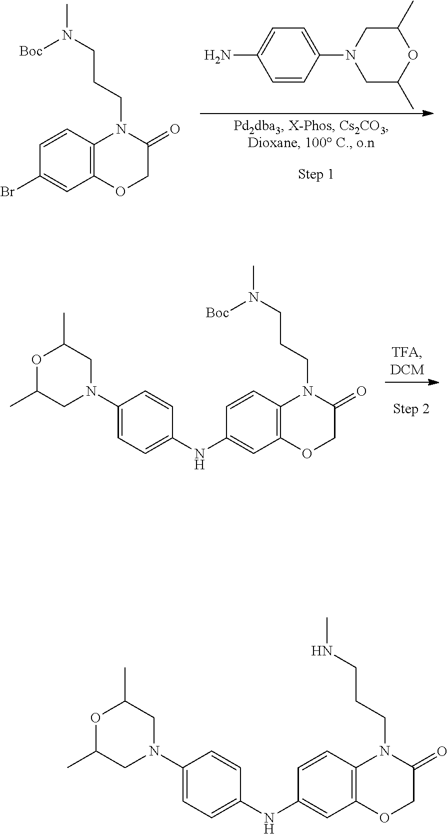 [SMILES: CN(CCCN1C(=O)COc2cc(Br)ccc21)C(=O)OC(C)(C)C ; CC1CN(c2ccc(N)cc2)CC(C)O1 ; CC1CN(c2ccc(Nc3ccc4c(c3)OCC(=O)N4CCCN(C)C(=O)OC(C)(C)C)cc2)CC(C)O1 ; CNCCCN1C(=O)COc2cc(Nc3ccc(N4CC(C)OC(C)C4)cc3)ccc21]

### Step 1. Preparation of 7-((4-(2,6-dimethylmorpholino)phenyl)amino)-4-(3-(methylamino)propyl)-2H-benzo[b][1,4]oxazin-3(4H)-one

To a solution of tert-butyl (3-(7-bromo-3-oxo-2,3-dihydro-4H-benzo[b][1,4]oxazin-4-yl)propyl)(methyl)carbamate (120 mg, 0.30 mmol) and 4-(2,6-dimethylmorpholino)aniline (68.2 mg, 0.33 mmol) in 1,4-dioxane (5 mL) was added Pd₂(dba)₃ (13.8 mg, 0.015 mmol), X-Phos (14.3 mg, 0.03 mmol) and Cs₂CO₃ (196 mg, 0.60 mmol) under nitrogen atmosphere. The mixture was stirred at 100° C. for 12 h. The reaction mixture was diluted with water (20 mL) and extracted with ethyl acetate (3×50 mL). The combined organic phase was dried and concentrated. The residue was purified by Prep-TLC (Petroleum ether:Ethyl acetate=1:5) to give the titled compound (75 mg, 47.6%) as a brown oil. LC-MS (m/z) 525.3 [M+H]⁺.

### Step 2. Preparation of 7-((4-(2,6-dimethylmorpholino)phenyl)amino)-4-(3-(methylamino)propyl)-2H-benzo[b][1,4]oxazin-3(4H)-one

To a solution of tert-butyl (3-(7-((4-(2,6-dimethylmorpholino) phenyl)amino)-3-oxo-2,3-dihydro-4H-benzo[b][1,4]oxazin-4-yl)propyl)(m ethyl)carbamate (75 mg, 0.14 mmol) in dichloromethane (5 mL) was added TFA (1.0 mL). The mixture was stirred at room temperature for 2 h. The reaction mixture was concentrated and adjusted PH to 7-8 with sat. NaHCO₃ and extracted with dichloromethane (3×50 mL). The combined organic phase was dried and concentrated. The residue was purified by Prep-TLC (Dichloromethane:Methanol:Ammonia=5:1:0.1) to give the titled compound 45 (27 mg, 44.5%) as a khaki solid. ¹H NMR (400 MHz, DMSO-d₆) δ 8.45 (s, 1H), 7.80 (s, 1H), 7.02 (d, J=8.8 Hz, 1H), 6.96-6.90 (m, 2H), 6.88-6.82 (m, 2H), 6.57 (dd, J=8.7, 2.5 Hz, 1H), 6.51 (d, J=2.5 Hz, 1H), 4.55 (s, 2H), 3.88 (t, J=7.0 Hz, 2H), 3.65 (ddd, J=10.3, 6.3, 2.3 Hz, 2H), 3.45-3.37 (m, 2H), 2.93-2.85 (m, 2H), 2.51 (s, 3H), 2.15 (dd, J=11.8, 10.3 Hz, 2H), 1.83 (q, J=7.5 Hz, 2H), 1.11 (d, J=6.2 Hz, 6H). LC-MS (m/z) 425.2 [M+H]⁺.

### Compound 46

### 4-(3-oxo-7-((4-(4-(trifluoromethyl)piperidin-1-yl)phenyl)amino)-2,3-dihydro-4H-benzo[b][1,4]oxazin-4-yl)butanamide

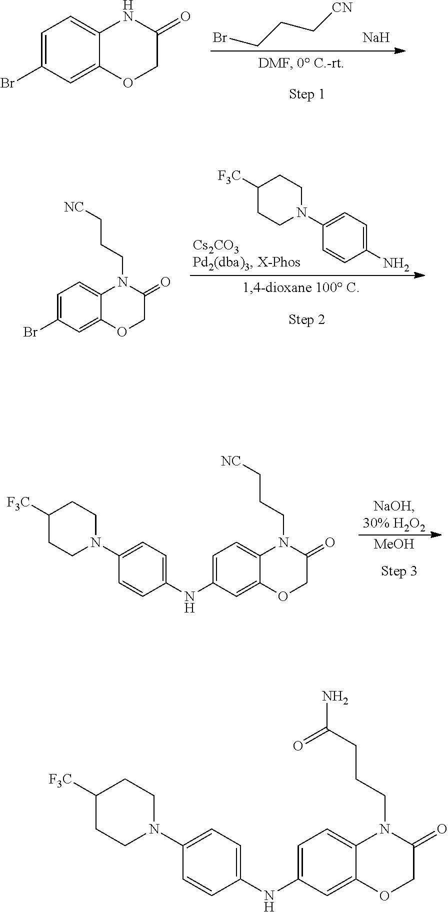

### Step 1. Preparation of 4-(7-bromo-3-oxo-2,3-dihydro-4H-benzo[b][1,4]oxazin-4-yl)butanenitrile

To a solution of 7-bromo-2H-1,4-benzoxazine-3(4H)-one (227 mg, 1 mmol) in DMF (10 mL) stirred at 0° C. under nitrogen atmosphere was added NaH (60%, 60 mg, 1.5 mmol). Then the mixture was stirred for 1 hour at 0° C. 4-Bromobutyronitrile (194 mg, 2 mmol) was added, the mixture was stirred overnight at r.t. 20 mL of water was added dropwise at 0° C. The precipitate was collected by filtration and washed with 10 mL of water. Target product was obtained as a yellow solid. (238 mg, 81.0%). Mass (m/z): 295.0 [M+H]⁺.

### Step 2. Preparation of 4-(3-oxo-7-((4-(4-(trifluoromethyl)piperidin-1-yl) phenyl)amino)-2,3-dihydro-4H-benzo[b][1,4]oxazin-4-yl)butanenitrile

The title compound (161 mg) as prepared in a total yield of 43.4% as a yellow solid from 4-(4-(trifluoromethyl)piperidin-1-yl)aniline (198 mg, 0.81 mmol), 4-(7-bromo-3-oxo-2,3-dihydro-4H-benzo[b][1,4]oxazin-4-yl) butanenitrile (238 mg, 0.81 mmol), Pd₂(dba)₃ (11.8 mg, 16 umol), X-Phos (18.5 mg, 32 umol), and Cs₂CO₃ (396 mg, 1.22 mmol) according to the procedure for compound 1. Mass (m/z): 459.2 [M+H]⁺.

### Step 3. Preparation of 4-(3-oxo-7-((4-(4-(trifluoromethyl)piperidin-1-yl) phenyl)amino)-2,3-dihydro-4H-benzo[b][1,4]oxazin-4-yl)butanamide (46)

To a solution of 4-(3-oxo-7-((4-(4-(trifluoromethyl)piperidin-1-yl) phenyl)amino)-2,3-dihydro-4H-benzo[b][1,4]oxazin-4-yl)butanenitrile in MeOH (3 mL) was added 1 mL of 10% NaOH solution at 0° C. Followed by the addition of 0.3 mL of 30% H₂02 solution then the mixture was stirred for 3 hours at r.t. 10 mL of water was added. The resulting solution was extracted with 3×10 mL of ethyl acetate. The organic layers were combined, washed with water (3×15 mL), dried and concentrated under vacuum. The residue was purified by prep-TLC (MeOH/DCM=1/10) to afford the desired product as a yellow solid (9.6 mg, 15.5%). ¹H NMR (400 MHz, DMSO-d₆) δ 7.78 (s, 1H), 7.27 (s, 1H), 7.04 (d, J=8.8 Hz, 1H), 6.95-6.91 (m, 2H), 6.89-6.83 (m, 2H), 6.74 (s, 1H), 6.58 (dd, J=8.9, 2.2 Hz, 1H), 6.51 (d, J=2.4 Hz, 1H), 4.51 (s, 2H), 3.77 (t, J=7.5 Hz, 2H), 3.62-3.55 (m, 2H), 2.60-2.53 (m, 2H), 2.43-2.37 (m, 1H), 2.08 (t, J=7.4 Hz, 2H), 1.88-1.82 (m, 2H), 1.74-1.66 (m, 2H), 1.60-1.44 (m, 2H). Mass (m/z): 477.2 [M+H]⁺.

### Compound 47

### 7-((4-(tert-butyl)phenyl)amino)-4-methyl-2H-benzo[b][1,4]oxazin-3(4H)-one

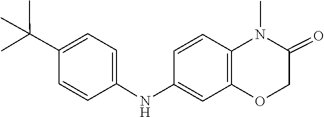 [SMILES: CN1C(=O)COc2cc(Nc3ccc(C(C)(C)C)cc3)ccc21]

The title compound 47 (26.0 mg) was prepared in a yield of 20.0% as a light blue solid from 7-bromo-4-methyl-2H-benzo[b][1,4]oxazin-3(4H)-one (100 mg, 0.42 mmol), 4-(tert-butyl)aniline (68 mg, 0.46 mmol), 1,4-dioxane (2 mL), Pd₂(dba)₃ (38 mg, 0.04 mmol), Xphos (39 mg, 0.08 mmol) and Cs₂CO₃ (277 mg, 0.83 mmol) according to the procedure for compound 1. ¹H NMR (400 MHz, DMSO-d₆) δ 7.99 (s, 1H), 7.26-7.16 (m, 2H), 7.00-6.89 (m, 3H), 6.70 (dd, J=8.7, 2.5 Hz, 1H), 6.61 (d, J=2.4 Hz, 1H), 4.55 (s, 2H), 3.20 (s, 3H), 1.22 (s, 9H). Mass (m/z): 310.2 [M+H]⁺.

### Compound 48

### 7-((4-(tert-butyl)phenyl)amino)-4-(2-(methylamino)ethyl)-2H-benzo[b][1,4]oxazin-3(4H)-one

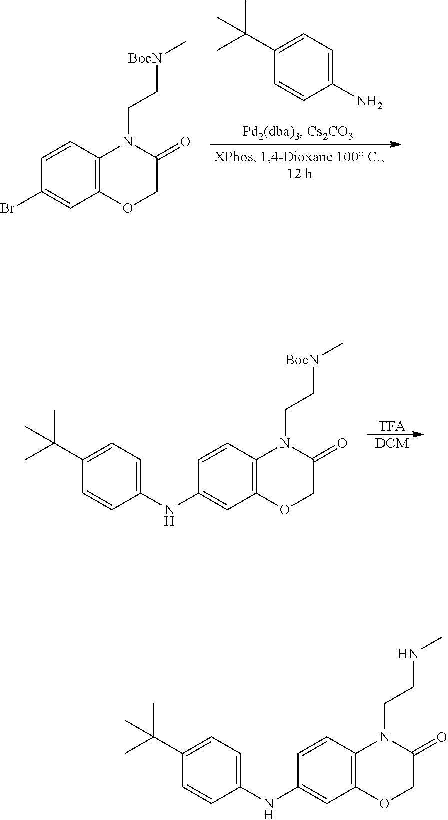

### Step 1. Preparation of tert-butyl (2-(7-((4-(tert-butyl)phenyl)amino)-3-oxo-2,3-dihydro-4H-benzo[b][1,4]oxazin-4-yl)ethyl)(methyl)carbamate

To a solution of tert-butyl (2-(7-bromo-3-oxo-2,3-dihydro-4H-benzo[b][1,4]oxazin-4-yl)ethyl)(methyl)carbamate (120 mg, 0.31 mmol) and 4-(tert-butyl)aniline (61 mg, 0.34 mmol) in 1,4-dioxane (2 mL) was added Pd₂(dba)₃ (28 mg, 0.03 mmol), Xphos (29 mg, 0.06 mmol) and Cs₂CO₃ (203 mg, 0.63 mmol) under nitrogen atmosphere. The mixture was stirred at 100° C. for 12 h, the mixture was purified by prep TLC to give tert-butyl (2-(7-((4-(tert-butyl)phenyl)amino)-3-oxo-2H-benzo[b][1,4]oxazin-4(3H)-yl)ethyl)(methyl)carbamate (50 mg, 35%) as a brown solid. Mass (m/z): 454.2 [M+H]⁺.

### Step 2. Preparation of 7-((4-(tert-butyl)phenyl)amino)-4-(2-(methylamino) ethyl)-2H-benzo[b][1,4]oxazin-3(4H)-one (48)

To a solution of tert-butyl (2-(7-((4-(tert-butyl)phenyl)amino)-3-oxo-2H-benzo[b][1,4]oxazin-4(3H)-yl)ethyl)(methyl)carbamate (50 mg, 0.11 mmol) in DCM (3 mL) was added 2,2,2-trifluoroacetic acid (1 mL). The mixture was stirred at r.t. for 1 h, the mixture was concentrated and purified by prep TLC to give 7-((4-(tert-butyl)phenyl)amino)-4-(2-(methylamino) ethyl)-2H-benzo[b][1,4]oxazin-3(4H)-one (16.9 mg, 43%) as a white solid. ¹H NMR (400 MHz, DMSO-d₆) δ 9.63 (s, 1H), 8.20 (s, 1H), 7.32 (d, J=8.6 Hz, 1H), 7.07-6.99 (m, 2H), 6.94-6.86 (m, 2H), 6.57 (dd, J=8.6, 1.9 Hz, 1H), 6.46 (d, J=1.8 Hz, 1H), 3.72-3.60 (m, 2H), 3.51-3.43 (m, 2H), 3.18 (s, 3H), 2.23-2.13 (m, 2H), 1.12 (d, J=6.2 Hz, 6H). Mass (m/z): 354.2 [M+H]⁺.

### Compound 49

### 7-((2,2-dimethyl-2,3-dihydro-1H-inden-5-yl)amino)-4-methyl-2H-benzo[b][1,4] oxazin-3(4H)-one

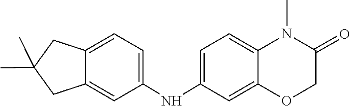 [SMILES: CN1C(=O)COc2cc(Nc3ccc4c(c3)CC(C)(C)C4)ccc21]

To a stirred solution of compound 5-bromo-2,2-dimethyl-2,3-dihydro-1H-indene (80 mg, 0.355 mmol) and 7-amino-4-methyl-2H-benzo[b][1,4]oxazin-3(4H)-one (69.66 mg, 0.391 mmol) in dioxane under N₂ was added NaOtBu (68.31 mg, 0.711 mmol), Pd₂(dba)₃ (32.54 mg, 0.036) and BINAP (44.26 mg, 0.071 mmol). The reaction mixture was stirred at 90° C. for 16 hrs. Then the mixture was filtered and concentrated. To the residue was added water (10 mL), extracted with EA (10 mL×3). The combined organic layers were dried over Na₂SO₄ and concentrated under vacuum. The residue was purified by prep-HPLC (column-Xbridge-C18, 150×21.2 mm, 5 um; Mobile phase: ACN-H₂O (0.1% trifluoroacetic acid (TFA)), 60%-80%) to afford compound 49 as a yellow solid (12.6 mg). ¹H NMR (400 MHz, CD₃OD) δ 6.99 (dd, J=13.4, 8.3 Hz, 2H), 6.88 (s, 1H), 6.82 (d, J=8.0 Hz, 1H), 6.71 (dd, J=8.7, 2.5 Hz, 1H), 6.65 (d, J=2.4 Hz, 1H), 4.54 (s, 2H), 3.32 (s, 3H), 2.65 (d, J=6.3 Hz, 4H), 1.14 (s, 6H). MS (m/z): 322.17 [M+H]⁺.

### Compound 50

### 7-((4-(2,6-dimethylmorpholino)-2-methylphenyl)amino)-4-(3-(methylamino)propyl)-2H-benzo[b][1,4]oxazin-3(4H)-one

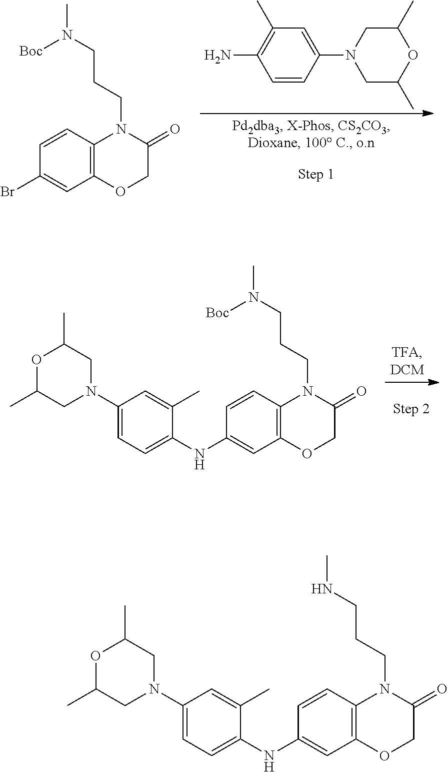 [SMILES: CN(CCCN1C(=O)COc2cc(Br)ccc21)C(=O)OC(C)(C)C ; Cc1cc(N2CC(C)OC(C)C2)ccc1N ; Cc1cc(N2CC(C)OC(C)C2)ccc1Nc1ccc2c(c1)OCC(=O)N2CCCN(C)C(=O)OC(C)(C)C ; CNCCCN1C(=O)COc2cc(Nc3ccc(N4CC(C)OC(C)C4)cc3C)ccc21]

The titled compound 50 (4.2 mg, 12.3%) as a white solid was prepared according to the procedure outlined for compound 48. ¹H NMR (400 MHz, DMSO-d₆) δ 8.41 (s, 1H), 7.22 (s, 1H), 6.96 (t, J=8.9 Hz, 2H), 6.81 (d, J=2.9 Hz, 1H), 6.72 (dd, J=8.7, 2.8 Hz, 1H), 6.31 (dd, J=8.7, 2.5 Hz, 1H), 6.23 (d, J=2.5 Hz, 1H), 4.52 (s, 2H), 3.86 (t, J=7.1 Hz, 1H), 3.70-3.65 (m, 1H), 3.65-3.53 (m, 1H), 3.51-3.43 (m, 2H), 2.93-2.80 (m, 2H), 2.50 (s, 3H), 2.27-2.10 (m, 2H), 2.09 (s, 3H), 1.89-1.76 (m, 1H), 1.21 (s, 2H), 1.11 (d, J=6.3 Hz, 6H). LC-MS (m/z) 439.2 [M+H]⁺.

### Compound 51

### 7-((4-(2,6-dimethylmorpholino)-2-methylphenyl)amino)-4-methyl-2H-benzo[b][1,4]oxazin-3(4H)-one

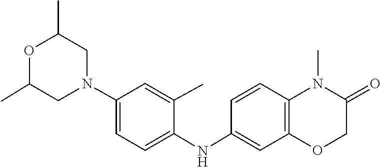 [SMILES: Cc1cc(N2CC(C)OC(C)C2)ccc1Nc1ccc2c(c1)OCC(=O)N2C]

The title compound 51 (95 mg) was prepared in a yield of 24.1% as a white solid from 7-bromo-4-methyl-2H-benzo[b][1,4]oxazin-3(4H)-one (250 mg, 1.03 mmol), 4-(2,6-dimethylmorpholino)-2-methylaniline (250.3 mg, 1.14 mmol), 1,4-dioxane (5 mL), Pd₂(dba)₃ (47.3 mg, 0.05 mmol), X-phos (49.2 mg, 0.103 mmol) and Cs₂CO₃ (674 mg, 2.06 mmol) according to the procedure of compound 1. ¹H NMR (400 MHz, DMSO-d₆) δ 7.17 (s, 1H), 6.95 (d, J=8.6 Hz, 1H), 6.87 (d, J=8.7 Hz, 1H), 6.80 (d, J=2.9 Hz, 1H), 6.71 (dd, J=8.6, 2.9 Hz, 1H), 6.30 (dd, J=8.7, 2.5 Hz, 1H), 6.22 (d, J=2.4 Hz, 1H), 4.50 (s, 2H), 3.68-3.61 (m, 2H), 3.47 (dt, J=10.7, 2.1 Hz, 2H), 3.16 (s, 3H), 2.17 (t, J=10.3 Hz, 2H), 2.08 (s, 3H), 1.11 (d, J=6.3 Hz, 6H). LC-MS (m/z) 382.2 [M+H]⁺.

### Compound 52

### 7-((4-(2,6-dimethylmorpholino)-3-methylphenyl)amino)-4-methyl-2H-benzo[b][1,4]oxazin-3(4H)-one

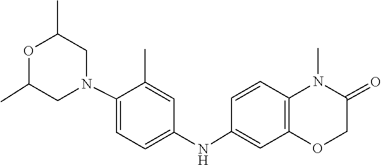 [SMILES: Cc1cc(Nc2ccc3c(c2)OCC(=O)N3C)ccc1N1CC(C)OC(C)C1]

The title compound 52 (37.4 mg) was prepared from in a yield of 23% as an off-white solid from 4-(2,6-dimethylmorpholino)-3-methylaniline (100 mg, 0.45 mmol), 7-bromo-4-methyl-2H-benzo[b][1,4]oxazin-3(4H)-one (120 mg, 0.50 mmol), 1,4-dioxane (2 mL), Pd₂(dba)₃ (41 mg, 0.05 mmol), Xphos (43 mg, 0.09 mmol) and Cs₂CO₃ (296 mg, 0.91 mmol) according to the procedure of compound 1. ¹H NMR (400 MHz, DMSO-d₆) δ 7.89 (s, 1H), 6.99 (d, J=8.7 Hz, 1H), 6.92 (d, J=8.3 Hz, 1H), 6.86 (s, 1H), 6.92-6.81 (m, 1H), 6.69 (dd, J=8.7, 2.5 Hz, 1H), 6.60 (d, J=2.5 Hz, 1H), 4.58 (s, 2H), 3.77-3.65 (m, 2H), 3.33 (s, 2H), 3.23 (s, 3H), 2.88-2.80 (m, 2H), 2.33-2.23 (m, 2H), 2.21 (s, 3H), 1.10 (d, J=6.2 Hz, 6H). Mass (m/z): 382.2 [M+H]⁺.

### Compound 53

### 7-((4-(2,6-dimethylmorpholino)phenyl)amino)-4-(2-(methylamino)ethyl)-2H-benzo[b][1,4]oxazin-3(4H)-one

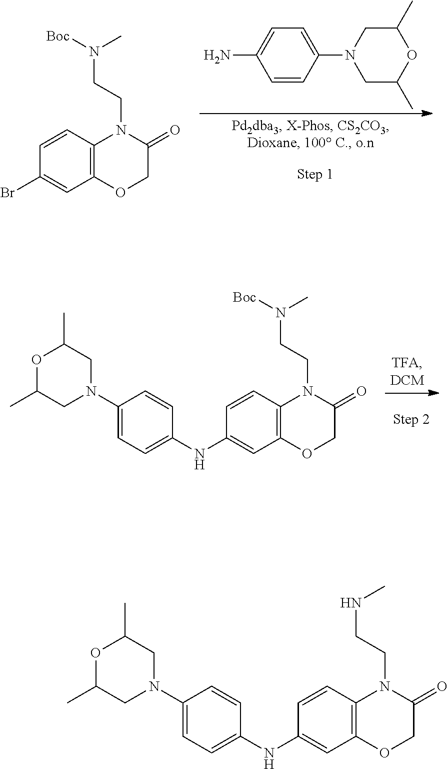 [SMILES: CN(CCN1C(=O)COc2cc(Br)ccc21)C(=O)OC(C)(C)C ; CC1CN(c2ccc(N)cc2)CC(C)O1 ; CC1CN(c2ccc(Nc3ccc4c(c3)OCC(=O)N4CCN(C)C(=O)OC(C)(C)C)cc2)CC(C)O1 ; CNCCN1C(=O)COc2cc(Nc3ccc(N4CC(C)OC(C)C4)cc3)ccc21]

### Step 1. Preparation of tert-butyl (2-(7-((4-(2,6-dimethylmorpholino) phenyl)amino)-3-oxo-2,3-dihydro-4H-benzo[b][1,4]oxazin-4-yl)ethyl)(met hyl)carbamate

To a solution of tert-butyl (2-(7-bromo-3-oxo-2,3-dihydro-4H-benzo[b][1,4]oxazin-4-yl)ethyl)(methyl)carbamate (120 mg, 0.31 mmol) and 4-(2,6-dimethylmorpholino)aniline (71.0 mg, 0.34 mmol) in 1,4-dioxane (5 mL) was added Pd₂(dba)₃ (14.3 mg, 0.016 mmol), X-phos (14.8 mg, 0.031 mmol) and Cs₂CO₃ (203 mg, 0.623 mmol) under nitrogen atmosphere. The mixture was stirred at 100° C. for 12 h. The reaction mixture was diluted with water (20 mL) and extracted with ethyl acetate (3×50 mL). The combined organic phase was dried and concentrated to give the titled compound (150 mg, crude) as a brown oil. LC-MS (m/z) 511.2 [M+H]⁺.

### Step 2. Preparation of 7-((4-(2,6-dimethylmorpholino)phenyl) amino)-4-(2-(methylamino)ethyl)-2H-benzo[b][1,4]oxazin-3(4H)-one

To a solution of tert-butyl (2-(7-((4-(2,6-dimethylmorpholino) phenyl)amino)-3-oxo-2,3-dihydro-4H-benzo[b][1,4]oxazin-4-yl)ethyl)(met hyl)carbamate (140 mg, 0.27 mmol) in dichloromethane (5 mL) was added trifluoroacetic acid (TFA, 1.0 mL). The mixture was stirred at room temperature for 2 h. The reaction mixture was concentrated and adjusted PH to 7-8 with sat. NaHCO₃ and extracted with dichloromethane (3×50 mL). The combined organic phase was dried and concentrated. The residue was purified by Prep-TLC (Petroleum ether:Ethyl acetate=1:5) and further purified by Prep-HPLC with the following conditions: Column: Spherical C18 40-60 um, 40 g; Mobile phase A: water (0.5% NH₄HCO₃); Mobile phase B: acetonitrile or ACN; Flow rate: 50 mL/min; Gradient: 30% B-65% B in 20 min; Detector: 254 nm. The fractions containing desired product were collected at 60% B and concentrated under reduce pressure to give the titled compound 53 (33.3 mg, 29.6%) as a white solid. ¹H NMR (400 MHz, DMSO-d₆) δ 7.78 (s, 1H), 7.04 (d, J=8.8 Hz, 1H), 7.01-6.92 (m, 2H), 6.92-6.84 (m, 2H), 6.60 (dd, J=8.7, 2.5 Hz, 1H), 6.52 (d, J=2.5 Hz, 1H), 4.54 (s, 2H), 3.89 (t, J=6.9 Hz, 2H), 3.72-3.64 (m, 2H), 3.44 (dt, J=10.8, 2.1 Hz, 2H), 2.63 (t, J=6.9 Hz, 2H), 2.28 (s, 3H), 2.19 (dd, J=11.8, 10.2 Hz, 2H), 1.14 (d, J=6.3 Hz, 6H). LC-MS (m/z) 411.2 [M+H]⁺.

### Compound 54

### 7-((4-(2,6-dimethyltetrahydro-2H-pyran-4-yl)phenyl)amino)-4-(2-(methylamino)ethyl)-2H-benzo[b][1,4]oxazin-3(4H)-one

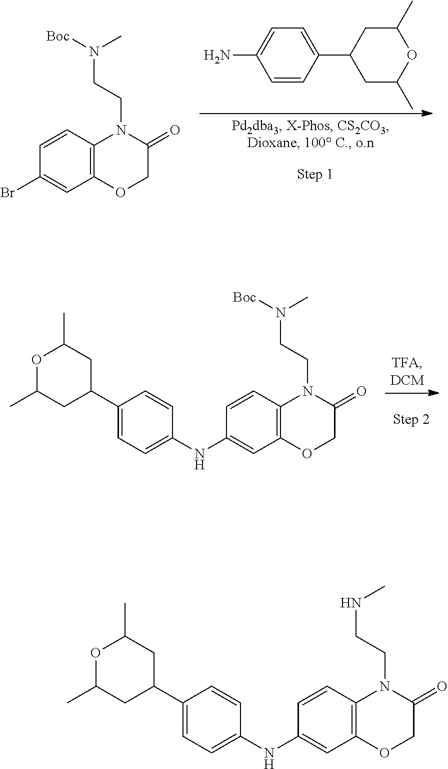 [SMILES: CN(CCN1C(=O)COc2cc(Br)ccc21)C(=O)OC(C)(C)C ; CC1CC(c2ccc(N)cc2)CC(C)O1 ; CC1CC(c2ccc(Nc3ccc4c(c3)OCC(=O)N4CCN(C)C(=O)OC(C)(C)C)cc2)CC(C)O1 ; CNCCN1C(=O)COc2cc(Nc3ccc(C4CC(C)OC(C)C4)cc3)ccc21]

The titled compound 54 (30.9 mg, 35%) as a white solid was prepared according to the procedure outlined for compound 53. ¹H NMR (400 MHz, DMSO-d₆) δ 8.03 (s, 1H), 7.09 (d, J=8.4 Hz, 3H), 7.03-6.93 (m, 2H), 6.72 (dd, J=8.7, 2.5 Hz, 1H), 6.64 (d, J=2.5 Hz, 1H), 4.56 (s, 2H), 3.90 (t, J=6.9 Hz, 2H), 3.59-3.46 (m, 2H), 2.76-2.61 (m, 3H), 2.28 (s, 3H), 1.75-1.66 (m, 2H), 1.25-1.04 (m, 8H). LC-MS (m/z) 410.2 [M+H]⁺.

### Compound 55

### 7-((4-(2,6-dimethylmorpholino)-2-methylphenyl)amino)-4-(2-(methylamino)ethyl)-2H-benzo[b][1,4]oxazin-3(4H)-one

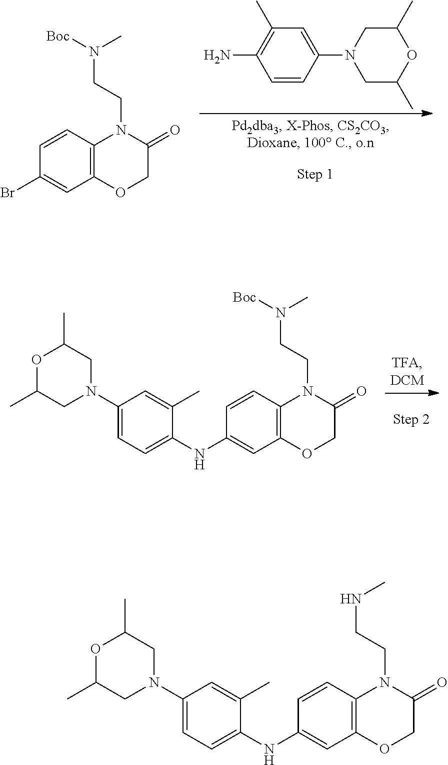 [SMILES: CN(CCN1C(=O)COc2cc(Br)ccc21)C(=O)OC(C)(C)C ; Cc1cc(N2CC(C)OC(C)C2)ccc1N ; Cc1cc(N2CC(C)OC(C)C2)ccc1Nc1ccc2c(c1)OCC(=O)N2CCN(C)C(=O)OC(C)(C)C ; CNCCN1C(=O)COc2cc(Nc3ccc(N4CC(C)OC(C)C4)cc3C)ccc21]

### Step 1. Preparation of tert-butyl (2-(7-((4-(2,6-dimethylmorpholino)-2-methylphenyl)amino)-3-oxo-2,3-dihydro-4H-benzo[b][1,4]oxazin-4-yl) ethyl)(methyl)carbamate

To a solution of tert-butyl (2-(7-bromo-3-oxo-2,3-dihydro-4H-benzo[b][1,4]oxazin-4-yl)ethyl)(methyl)carbamate (120 mg, 0.31 mmol) and 4-(2,6-dimethylmorpholino)-2-methylaniline (75.5 mg, 0.34 mmol) in 1,4-dioxane (5 mL) was added Pd₂(dba)₃ (14.3 mg, 0.016 mmol), X-phos (14.8 mg, 0.031 mmol) and Cs₂CO₃ (203 mg, 0.623 mmol) under nitrogen atmosphere. The mixture was stirred at 100° C. for 12 h. The reaction mixture was diluted with water (20 mL) and extracted with ethyl acetate (3×50 mL). The combined organic phase was dried and concentrated to give the titled compound (120 mg, crude) as a brown oil. LC-MS (m/z) 525.3 [M+H]⁺.

### Step 2. Preparation of 7-((4-(2,6-dimethylmorpholino)-2-methylphenyl) amino)-4-(2-(methylamino)ethyl)-2H-benzo[b][1,4]oxazin-3(4H)-one

To a solution of tert-butyl (2-(7-((4-(2,6-dimethylmorpholino)-2-methylphenyl)amino)-3-oxo-2,3-dihydro-4H-benzo[b][1,4]oxazin-4-yl) ethyl)(methyl)carbamate (120 mg, 0.23 mmol) in dichloromethane (5 mL) was added TFA (1.0 mL) The mixture was stirred at room temperature for 2 h. The reaction mixture was concentrated and adjusted pH to 7-8 with sat. NaHCO₃ and extracted with dichloromethane (3×50 mL). The combined organic phase was dried and concentrated. The residue was purified by Prep-HPLC with the following conditions: Column: Spherical C18 40-60 um, 40 g; Mobile phase A: water (0.5% NH₄HCO₃); Mobile phase B: acetonitrile or ACN; Flow rate: 50 mL/min; Gradient: 30% B-65% B in 20 min; Detector: 254 nm. The fractions containing desired product were collected at 57% B and concentrated under reduce pressure to give the titled compound 55 (25 mg, 25.7%) as a white solid. ¹H NMR (400 MHz, DMSO-d₆) δ 7.19 (s, 1H), 6.99 (d, J=8.7 Hz, 2H), 6.83 (d, J=2.8 Hz, 1H), 6.75 (dd, J=8.7, 2.9 Hz, 1H), 6.34 (dd, J=8.7, 2.5 Hz, 1H), 6.25 (d, J=2.5 Hz, 1H), 4.51 (s, 2H), 3.88 (t, J=6.9 Hz, 2H), 3.68 (dtt, J=12.4, 6.2, 3.7 Hz, 2H), 3.54-3.46 (m, 2H), 2.65 (t, J=7.0 Hz, 2H), 2.29 (s, 3H), 2.20 (dd, J=11.9, 10.2 Hz, 2H), 2.12 (s, 3H), 1.15 (d, J=6.2 Hz, 6H). LC-MS (m/z) 425.2 [M+H]⁺.

### Compound 56

### 7-((4-(2,6-dimethylmorpholino)-3-methylphenyl)amino)-4-(3-(methylamino)propyl)-2H-benzo[b][1,4]oxazin-3(4H)-one

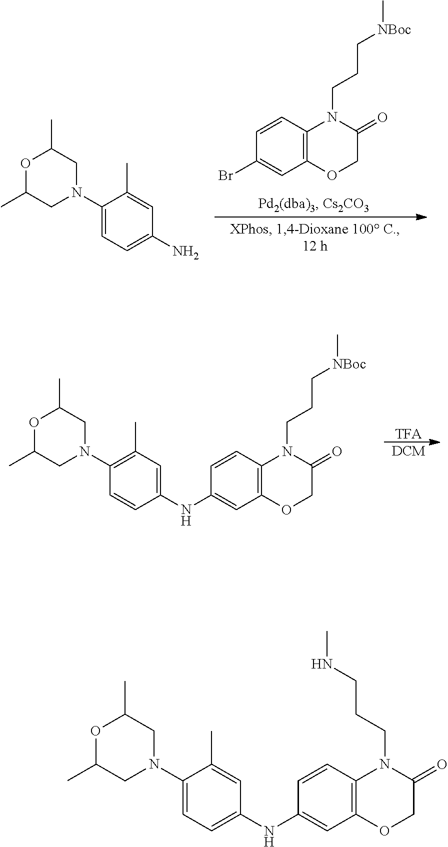

### Step 1. Preparation of tert-butyl (3-(7-((4-(2,6-dimethylmorpholino)-3-methylphenyl)amino)-3-oxo-2H-benzo[b][1,4]oxazin-4(3H)-yl)propyl) (methyl)carbamate

To a solution of tert-butyl (3-(7-bromo-3-oxo-2H-benzo[b][1,4]oxazin-4(3H)-yl)propyl)(methyl)carbamate (150 mg, 0.37 mmol) and 4-(2,6-dimethylmorpholino)-3-methylaniline (75 mg, 0.34 mmol) in 1,4-dioxane (2 mL) was added Pd₂(dba)₃ (31 mg, 0.03 mmol), Xphos (29 mg, 0.07 mmol) and Cs₂CO₃ (222 mg, 0.68 mmol) under nitrogen atmosphere. The mixture was stirred at 100° C. for 12 h, the mixture was purified by prep TLC to give tert-butyl (3-(7-((4-(2,6-dimethylmorpholino)-3-methylphenyl)amino)-3-oxo-2H-benzo[b][1,4]oxa zin-4(3H)-yl)propyl)(methyl)carbamate (50 mg, 27%) as a brown solid. Mass (m/z): 539.3 [M+H]⁺.

### Step 2. Preparation of 7-((4-(2,6-dimethylmorpholino)-3-methylphenyl) amino)-4-(3-(methylamino)propyl)-2H-benzo[b][1,4]oxazin-3(4H)-one

To a solution of tert-butyl (3-(7-((4-(2,6-dimethylmorpholino)-3-methylphenyl)amino)-3-oxo-2H-benzo[b][1,4]oxazin-4(3H)-yl)propyl)(methyl)carbamate (50 mg, 0.09 mmol) in DCM (3 mL) was added 2,2,2-trifluoroacetic acid (1 mL). The mixture was stirred at r.t. for 1 h, the mixture was concentrated and purified by prep HPLC to give 7-((4-(2,6-dimethylmorpholino)-3-methylphenyl)amino)-4-(3-(methylamin o)propyl)-2H-benzo[b][1,4]oxazin-3(4H)-one (16.8 mg, 41%) as an off-white solid. ¹H NMR (400 MHz, DMSO-d₆) δ 7.87 (s, 1H), 7.03 (d, J=8.8 Hz, 1H), 6.89 (d, J=8.3 Hz, 1H), 6.86-6.77 (m, 2H), 6.68-6.61 (m, 1H), 6.56 (d, J=2.5 Hz, 1H), 4.53 (s, 2H), 3.85 (t, J=7.2 Hz, 2H), 3.74-3.62 (m, 2H), 2.85-2.74 (m, 2H), 2.54-2.48 (m, 2H), 2.26 (s, 3H), 2.29-2.19 (m, 2H), 2.17 (s, 3H), 1.66 (p, J=7.1 Hz, 2H), 1.07 (d, J=6.3 Hz, 6H). Mass (m/z): 439.3 [M+H]⁺.

### Compound 57

### 7-((3,5-difluoro-4-(4-(trifluoromethyl)piperidin-1-yl)phenyl)amino)-4-methyl-2H-benzo[b][1,4]oxazin-3(4H)-one

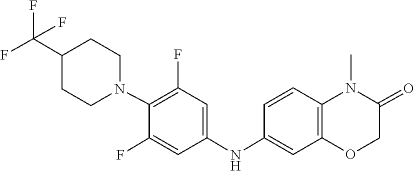 [SMILES: CN1C(=O)COc2cc(Nc3cc(F)c(N4CCC(C(F)(F)F)CC4)c(F)c3)ccc21]

The title compound 57 (108.7 mg) was prepared in a yield of 34.0% as an off-white solid from 7-bromo-4-methyl-2H-benzo[b][1,4]oxazin-3(4H)-one (190 mg, 0.79 mmol), 3,5-difluoro-4-(4-(trifluoromethyl) piperidin-1-yl)aniline (202 mg, 0.72 mmol), 1,4-dioxane (2 mL), Pd₂(dba)₃ (66 mg, 0.07 mmol), Xphos (68 mg, 0.14 mmol) and Cs₂CO₃ (470 mg, 1.44 mmol) according to the procedure for compound 1. ¹H NMR (400 MHz, DMSO-d₆) δ 8.37 (s, 1H), 7.04 (d, J=8.7 Hz, 1H), 6.77 (dd, J=8.7, 2.5 Hz, 1H), 6.67 (d, J=2.4 Hz, 1H), 6.60-6.47 (m, 2H), 4.59 (s, 2H), 3.22 (s, 3H), 3.09-2.91 (m, 4H), 2.45-2.32 (m, 1H), 1.84-1.75 (m, 2H), 1.50 (qd, J=12.2, 4.9 Hz, 2H). Mass (m/z): 442.4 [M+H]⁺.

### Compound 58

### 4-methyl-7-((2-(4-(trifluoromethyl)piperidin-1-yl)pyrimidin-5-yl) amino)-2H-benzo[b][1,4]oxazin-3(4H)-one

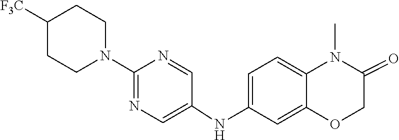 [SMILES: CN1C(=O)COc2cc(Nc3cnc(N4CCC(C(F)(F)F)CC4)nc3)ccc21]

The title compound 58 (56.6 mg) was prepared in a yield of 30.6% as a white solid from 7-bromo-4-methyl-2H-benzo[b][1,4]oxazin-3(4H)-one (110 mg, 0.45 mmol), 2-(4-(trifluoromethyl) piperidin-1-yl) pyrimidin-5-amine (112 mg, 0.45 mmol), 1,4-dioxane (5 mL), Pd₂(dba)₃ (20.8 mg, 0.023 mmol), X-phos (21.6 mg, 0.045 mmol) and Cs₂CO₃ (296.3 mg, 0.91 mmol) according to the procedure for compound 1. ¹H NMR (400 MHz, DMSO-d₆) δ 8.25 (s, 2H), 7.70 (s, 1H), 6.96 (d, J=8.7 Hz, 1H), 6.49 (dd, J=8.6, 2.5 Hz, 1H), 6.41 (d, J=2.5 Hz, 1H), 4.69 (d, J=13.3 Hz, 2H), 4.56 (s, 2H), 3.21 (s, 3H), 2.89 (td, J=13.0, 2.6 Hz, 2H), 2.63 (dq, J=7.8, 4.2, 3.6 Hz, 1H), 1.87 (dd, J=13.6, 3.6 Hz, 2H), 1.38 (qd, J=12.5, 4.3 Hz, 2H). LC-MS (m/z) 408.2 [M+H]⁺.

### Compound 59

### 7-((5-fluoro-2-methyl-4-(4-(trifluoromethyl)piperidin-1-yl)phenyl)amino)-4-methyl-2H-benzo[b][1,4]oxazin-3(4H)-one

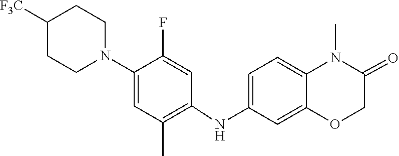 [SMILES: Cc1cc(N2CCC(C(F)(F)F)CC2)c(F)cc1Nc1ccc2c(c1)OCC(=O)N2C]

The title compound 59 (67.5 mg) was prepared in a yield of 30.6% as a light yellow solid from 7-bromo-4-methyl-2H-benzo[b][1,4]oxazin-3(4H)-one (110 mg, 0.45 mmol), 2-(4-(trifluoromethyl)piperidin-1-yl) pyrimidin-5-amine (125.5 mg, 0.45 mmol), 1,4-dioxane (5 mL), Pd₂(dba)₃ (20.8 mg, 0.023 mmol), X-phos (21.6 mg, 0.045 mmol) and Cs₂CO₃ (296.3 mg, 0.91 mmol) according to the procedure for compound 1. ¹H NMR (400 MHz, DMSO-d₆) δ 7.35 (s, 1H), 6.99 (d, J=8.7 Hz, 1H), 6.91-6.81 (m, 2H), 6.53 (dd, J=8.7, 2.5 Hz, 1H), 6.46 (d, J=2.4 Hz, 1H), 4.57 (s, 2H), 3.35 (s, 1H), 3.32 (s, 1H), 3.22 (s, 3H), 2.67 (td, J=12.1, 2.4 Hz, 2H), 2.47-2.38 (m, 1H), 2.12 (s, 3H), 1.90 (dd, J=12.1, 3.2 Hz, 2H), 1.60 (qd, J=12.4, 4.0 Hz, 2H). LC-MS (m/z) 438.2 [M+H]⁺.

### Compound 60

### N-(4-(2,6-dimethylmorpholino)phenyl)-4-methyl-3,4-dihydro-2H-benzo[b][1,4]oxazin-7-amine

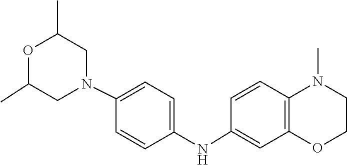 [SMILES: CC1CN(c2ccc(Nc3ccc4c(c3)OCCN4C)cc2)CC(C)O1]

The title compound 60 (18 mg) was prepared in a yield of 5% as a grey solid from 7-bromo-4-methyl-3,4-dihydro-2H-benzo[b][1,4]oxazine (121 mg, 0.53 mmol), 4-(2,6-dimethylmorpholino)aniline (100 mg, 0.48 mmol), 1,4-dioxane (2 mL), Pd₂(dba)₃ (44 mg, 0.05 mmol), Xphos (46 mg, 0.10 mmol) and Cs₂CO₃ (316 mg, 0.97 mmol) according to the procedure for compound 1. ¹H NMR (400 MHz, Pyr) δ 8.16 (s, 1H), 7.40-7.32 (m, 2H), 7.06 (d, J=2.5 Hz, 1H), 7.06-6.99 (m, 2H), 6.97 (dd, J=8.6, 2.5 Hz, 1H), 6.78 (d, J=8.5 Hz, 1H), 4.27-4.21 (m, 2H), 3.86-3.74 (m, 2H), 3.45-3.37 (m, 2H), 3.03 (t, J=4.4 Hz, 2H), 2.71 (s, 3H), 2.35 (t, J=10.9 Hz, 2H), 1.21 (d, J=6.2 Hz, 6H). Mass (m/z): 354.4 [M+H]⁺.

### Compound 61

### 7-((4-(2,6-dimethylmorpholino)-5-fluoro-2-methylphenyl)amino)-4-methyl-2H-benzo[b][1,4]oxazin-3(4H)-one

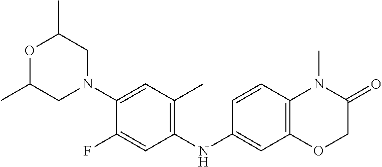 [SMILES: Cc1cc(N2CC(C)OC(C)C2)c(F)cc1Nc1ccc2c(c1)OCC(=O)N2C]

The title compound 61 (77 mg) was prepared in a yield of 45% as a white solid from 7-bromo-4-methyl-2H-benzo[b][1,4]oxazin-3(4H)-one (121 mg, 0.50 mmol), 4-(2,6-dimethylmorpholino)-5-fluoro-2-methylaniline (100 mg, 0.42 mmol), 1,4-dioxane (2 mL), Pd₂(dba)₃ (38 mg, 0.04 mmol), Xphos (40 mg, 0.08 mmol) and Cs₂CO₃ (274 mg, 0.84 mmol) according to the procedure for compound 1. ¹H NMR (400 MHz, DMSO) δ 7.34 (s, 1H), 6.98 (d, J=8.7 Hz, 1H), 6.91-6.81 (m, 2H), 6.52 (dd, J=8.7, 2.5 Hz, 1H), 6.44 (d, J=2.4 Hz, 1H), 4.57 (s, 2H), 3.79-3.67 (m, 2H), 3.22 (s, 3H), 3.19-3.11 (m, 2H), 2.37-2.27 (m, 2H), 2.12 (s, 3H), 1.11 (d, J=6.2 Hz, 6H). Mass (m/z): 400.4 [M+H]⁺.

### Compound 62

### 7-((4-(2,6-dimethylmorpholino)-3-methylphenyl)amino)-4-(2-(methylamino)ethyl)-2H-benzo[b][1,4]oxazin-3(4H)-one

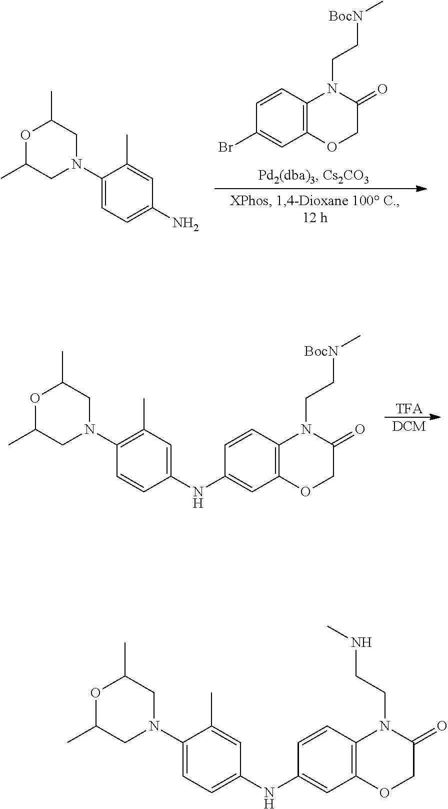

### Step 1. Preparation of tert-butyl (2-(7-((4-(2,6-dimethylmorpholino)-3-methylphenyl)amino)-3-oxo-2H-benzo[b][1,4]oxazin-4(3H)-yl)ethyl)(met hyl)carbamate

To a solution of tert-butyl (2-(7-bromo-3-oxo-2H-benzo[b][1,4]oxazin-4(3H)-yl)ethyl)(methyl)carbamate (146 mg, 0.38 mmol) and 4-(2,6-dimethylmorpholino)-3-methylaniline (70 mg, 0.32 mmol) in 1,4-dioxane (2 mL) was added Pd₂(dba)₃ (29 mg, 0.03 mmol), Xphos (30 mg, 0.06 mmol) and Cs₂CO₃ (207 mg, 0.63 mmol) under nitrogen atmosphere. The mixture was stirred at 100° C. for 12 h, the mixture was purified by prep TLC to give tert-butyl (2-(7-((4-(2,6-dimethylmorpholino)-3-methylphenyl)amino)-3-oxo-2H-benzo[b][1,4]oxa zin-4(3H)-yl)ethyl)(methyl)carbamate (50 mg, 30%) as a brown solid. Mass (m/z): 525.3 [M+H]⁺.

### Step 2. Preparation of 7-((4-(2,6-dimethylmorpholino)-3-methylphenyl) amino)-4-(2-(methylamino)ethyl)-2H-benzo[b][1,4]oxazin-3(4H)-one (62)

To a solution of tert-butyl (2-(7-((4-(2,6-dimethylmorpholino)-3-methylphenyl)amino)-3-oxo-2H-benzo[b][1,4]oxazin-4(3H)-yl)ethyl)(methyl)carbamate (50 mg, 0.09 mmol) in DCM (3 mL) was added 2,2,2-trifluoroacetic acid (1 mL). The mixture was stirred at r.t. for 1 h, the mixture was concentrated and purified by prep HPLC to give 7-((4-(2,6-dimethylmorpholino)-3-methylphenyl)amino)-4-(2-(methylami no)ethyl)-2H-benzo[b][1,4]oxazin-3(4H)-one (20 mg, 49%) as an off-white solid. ¹H NMR (400 MHz, DMSO-d₆) δ 7.86 (s, 1H), 7.03 (d, J=8.8 Hz, 1H), 6.92-6.77 (m, 3H), 6.64 (dd, J=8.7, 2.5 Hz, 1H), 6.56 (d, J=2.4 Hz, 1H), 4.52 (s, 2H), 3.86 (t, J=6.9 Hz, 2H), 3.74-3.62 (m, 2H), 2.85-2.74 (m, 2H), 2.60 (d, J=6.9 Hz, 2H), 2.29-2.15 (m, 8H), 1.07 (d, J=6.2 Hz, 6H). Mass (m/z): 425.3 [M+H]⁺.

### Compound 63

### 7-((3-fluoro-2-methyl-4-(4-(trifluoromethyl)piperidin-1-yl)phenyl) amino)-4-methyl-2H-benzo[b][1,4]oxazin-3(4H)-one

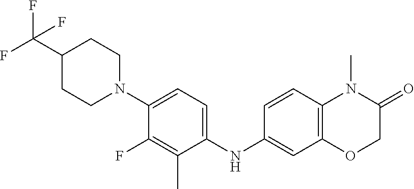 [SMILES: Cc1c(Nc2ccc3c(c2)OCC(=O)N3C)ccc(N2CCC(C(F)(F)F)CC2)c1F]

The title compound 63 (127.7 mg) was prepared in a yield of 40% as a pink solid from 7-bromo-4-methyl-2H-benzo[b][1,4]oxazin-3(4H)-one (191 mg, 0.80 mmol), 3-fluoro-2-methyl-4-(4-(trifluoromethyl)piperidin-1-yl)aniline (200 mg, 0.72 mmol), 1,4-dioxane (2 mL), Pd₂(dba)₃ (66 mg, 0.07 mmol), Xphos (68 mg, 0.14 mmol) and Cs₂CO₃ (472 mg, 1.45 mmol) according to the procedure for compound 1. ¹H NMR (400 MHz, DMSO-d₆) δ 7.40 (s, 1H), 6.92 (d, J=8.7 Hz, 1H), 6.85 (d, J=8.8 Hz, 1H), 6.85-6.76 (m, 1H), 6.44 (dd, J=8.6, 2.5 Hz, 1H), 6.37 (d, J=2.4 Hz, 1H), 4.52 (s, 2H), 3.34-3.31 (m, 1H), 3.29-3.26 (m, 1H), 3.18 (s, 3H), 2.68-2.57 (m, 2H), 2.45-2.34 (m, 1H), 2.02 (d, J=2.5 Hz, 3H), 1.91-1.82 (m, 2H), 1.65-1.50 (m, 2H). Mass (m/z): 438.2 [M+H]⁺.

### Compound 64

### 7-((4-(2,6-dimethyltetrahydro-2H-pyran-4-yl)phenyl)amino)-4-methyl-2H-benzo[b][1,4]oxazin-3(4H)-one

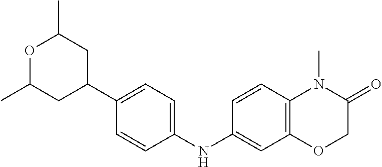 [SMILES: CC1CC(c2ccc(Nc3ccc4c(c3)OCC(=O)N4C)cc2)CC(C)O1]

The title compound 64 (56 mg) was prepared in a yield of 44% as a light yellow solid from 7-bromo-4-methyl-2H-benzo[b][1,4]oxazin-3(4H)-one (90 mg, 0.38 mmol), 4-(2,6-dimethyltetrahydro-2H-pyran-4-yl)aniline (70 mg, 0.34 mmol), 1,4-dioxane (2 mL), Pd₂(dba)₃ (31 mg, 0.03 mmol), Xphos (31 mg, 0.07 mmol) and Cs₂CO₃ (223 mg, 0.68 mmol) according to the procedure for compound 1. ¹H NMR (400 MHz, DMSO-D₆) δ 8.00 (s, 1H), 7.10-7.02 (m, 2H), 6.97 (d, J=8.7 Hz, 1H), 6.97-6.89 (m, 2H), 6.70 (dd, J=8.7, 2.5 Hz, 1H), 6.61 (d, J=2.4 Hz, 1H), 4.55 (s, 2H), 3.55-3.43 (m, 2H), 3.20 (s, 3H), 2.73-2.62 (m, 1H), 1.71-1.62 (m, 2H), 1.22-1.12 (m, 2H), 1.09 (d, J=6.1 Hz, 6H). Mass (m/z): 367.4 [M+H]⁺.

### Compound 65

### 7-((2-(2,6-dimethylmorpholino)quinolin-6-yl)amino)-4-methyl-2H-benzo[b][1,4]oxazin-3(4H)-one

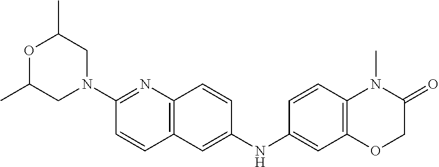 [SMILES: CC1CN(c2ccc3cc(Nc4ccc5c(c4)OCC(=O)N5C)ccc3n2)CC(C)O1]

The title compound 65 (66.5 mg) was prepared in a yield of 27% as a yellow solid from 7-bromo-4-methyl-2H-benzo[b][1,4]oxazin-3(4H)-one (154 mg, 0.64 mmol), 2-(2,6-dimethylmorpholino)quinolin-6-amine (150 mg, 0.58 mmol), 1,4-dioxane (2 mL), Pd₂(dba)₃ (53 mg, 0.06 mmol), Xphos (55 mg, 0.12 mmol) and Cs₂CO₃ (381 mg, 1.17 mmol) according to the procedure for compound 1. ¹H NMR (400 MHz, DMSO-d₆) δ 8.21 (s, 1H), 7.93 (d, J=9.1 Hz, 1H), 7.52 (d, J=8.8 Hz, 1H), 7.34-7.25 (m, 2H), 7.18 (d, J=9.2 Hz, 1H), 7.05 (d, J=8.7 Hz, 1H), 6.82 (dd, J=8.7, 2.5 Hz, 1H), 6.73 (d, J=2.4 Hz, 1H), 4.61 (s, 2H), 4.33-4.24 (m, 2H), 3.69-3.59 (m, 2H), 3.25 (s, 3H), 2.48-2.40 (m, 2H), 1.18 (d, J=6.2 Hz, 6H). Mass (m/z): 419.4 [M+H]⁺.

### Compound 66

### 7-((2-(2,6-dimethylmorpholino)pyrimidin-5-yl)amino)-4-methyl-2H-benzo[b][1,4]oxazin-3(4H)-one

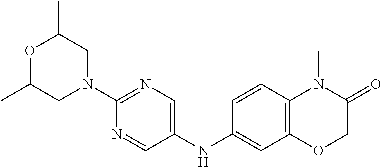 [SMILES: CC1CN(c2ncc(Nc3ccc4c(c3)OCC(=O)N4C)cn2)CC(C)O1]

The title compound 66 (61.5 mg) was prepared in a yield of 43.4% as a green solid from 2-(2,6-dimethylmorpholino)pyrimidin-5-amine (80 mg, 0.385 mmol), 7-bromo-4-methyl-2H-benzo[b][1,4]oxazin-3(4H)-one (92.7 mg, 0.385 mmol), dioxane (15 mL), Pd₂(dba)₃ (17.6 mg, 0.0193 mmol), X-phos (18.4 mg, 0.0385 mmol) and Cs₂CO₃ (188 mg, 0.578 mmol) according to the procedure for compound 1. ¹H NMR (400 MHz, DMSO) δ 8.25 (d, J=5.0 Hz, 2H), 7.69 (d, J=10.3 Hz, 1H), 6.96 (d, J=8.7 Hz, 1H), 6.48 (dd, J=8.7, 2.5 Hz, 1H), 6.41 (d, J=2.5 Hz, 1H), 4.56 (s, 2H), 4.40 (dd, J=13.0, 1.7 Hz, 2H), 3.64-3.46 (m, 2H), 3.21 (s, 3H), 2.46 (s, 1H), 2.08 (s, 1H), 1.15 (d, J=6.2 Hz, 6H). Mass (m/z): 370.4 [M+H]⁺

### Compound 67

### 4-methyl-7-((4-(tert-pentyl)phenyl)amino)-2H-benzo[b][1,4]oxazin-3(4H)-one

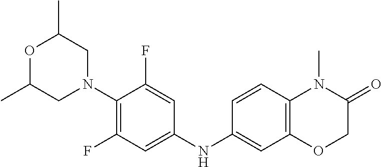 [SMILES: CC1CN(c2c(F)cc(Nc3ccc4c(c3)OCC(=O)N4C)cc2F)CC(C)O1]

The title compound 67 (69 mg) was prepared in a yield of 41.6% as a yellow solid from 4-(2,6-dimethylmorpholino)-3,5-difluoroaniline (100 mg, 0.413 mmol), 7-bromo-4-methyl-2H-benzo[b][1,4]oxazin-3(4H)-one (99.6 mg, 0.413 mmol), dioxane (15 mL), Pd₂(dba)₃ (18.9 mg, 0.021 mmol), X-phos (19.7 mg, 0.0413 mmol) and Cs₂CO₃ (202 mg, 0.619 mmol) according to the procedure for compound 1. ¹H NMR (400 MHz, DMSO) δ 8.41 (s, 1H), 7.08 (d, J=8.7 Hz, 1H), 6.81 (dd, J=8.6, 2.4 Hz, 1H), 6.71 (d, J=2.4 Hz, 1H), 6.64-6.51 (m, 2H), 4.63 (s, 2H), 3.65 (dd, J=11.4, 4.6 Hz, 2H), 3.25 (s, 3H), 2.86 (d, J=10.0 Hz, 2H), 2.68 (t, J=10.7 Hz, 2H), 1.06 (d, J=6.3 Hz, 6H). Mass (m/z): 404.4 [M+H]⁺

### Compound 68

### 2-ethyl-4-methyl-7-((4-(4-(trifluoromethyl)piperidin-1-yl)phenyl) amino)-2H-benzo[b][1,4]oxazin-3(4H)-one

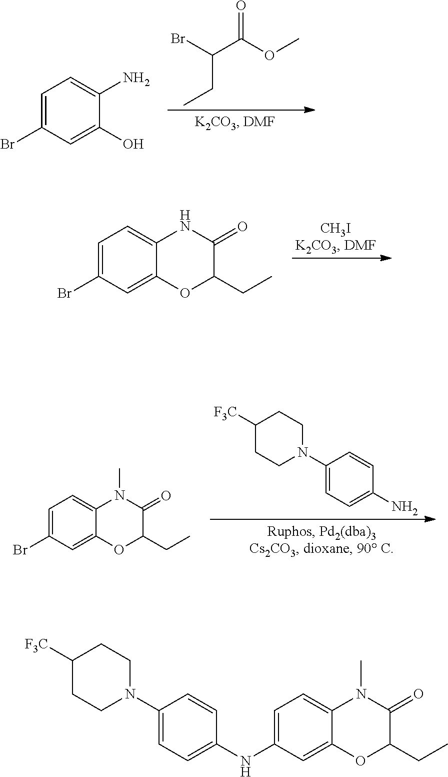

### Step 1. Preparation of 7-bromo-2-ethyl-2H-benzo[b][1,4]oxazin-3(4H)-one

To a solution of compound 2-amino-5-bromophenol (200 mg, 1.06 mmol) in DMF (5 mL) was added K₂CO₃ (367.53 mg, 2.659 mmol) and methyl 2-bromobutanoate (231 g, 1.28 mmol) at r.t. Then the mixture was stirred at 90° C. for 16 hrs. After cooling to rt, the reaction solution was diluted with water (20 mL), extracted with ethyl acetate (20 mL*3). The organic layers were combined, washed with brine (20 mL), concentrated under vacuum and the residue was purified by silica gel chromatography (PE:EA=5:1) to give the title compound as a white solid (220 mg, 80.8%). Mass (m/z): 255.7 [M+H]⁺.

### Step 2. Preparation of 7-bromo-2-ethyl-4-methyl-2H-benzo[b][1,4]oxazin-3(4H)-one

To a solution of 7-bromo-2-ethyl-2H-benzo[b][1,4]oxazin-3(4H)-one (220 mg, 0.86 mmol) in DMF (5 mL) was added K₂CO₃ (118.7 mg, 0.859 mmol) and Mel (365.82 mg, 2.57 mmol) at 25° C. Then the mixture was stirred at 25° C. for 2 hrs, diluted with water (20 mL), extracted with ethyl acetate (20 mL*3), the organic layers were combined, washed with brine (20 mL), concentrated under vacuum to give the title compound as a white solid (220 mg, 74.49%). Mass (m/z): 269.7 [M+H]⁺.

### Step 3. Preparation 2-ethyl-4-methyl-7-((4-(4-(trifluoromethyl) piperidin-1-yl)phenyl)amino)-2H-benzo[b][1,4]oxazin-3(4H)-one (68)

To a solution of compound 7-bromo-2-ethyl-4-methyl-2H-benzo[b][1,4]oxazin-3(4H)-one

To a solution of 7-bromo-2-ethyl-2H-benzo[b][1,4]oxazin-3(4H)-one (100 mg, 0.37 mmol), compound 4-(4-(trifluoromethyl)piperidin-1-yl)aniline (108 mg, 0.444 mmol), Cs₂CO₃ (241 mg, 0.74 mmol) and Ruphos (17 mg, 0.037 mmol) in 1,4-dioxane (15 mL) was added Pd₂(dba)₃ (68 mg, 0.074 mmol). The mixture was stirred at 90° C. for 3 hours under N₂ atmosphere. After cooling to ambient temperature, ethyl acetate (20 mL) was added to the reaction mixture and the mixture was filtered through celite. The filtrate was washed with water and saturated brine. The organic layer was dried over magnesium sulfate and concentrated under reduced pressure.

The residue was purified by silica gel column to provide 8 mg of compound 68 as a yellow solid. ¹H NMR (400 MHz, DMSO-d⁶) δ 7.82 (s, 1H), 6.96 (m, 5H), 6.59 (d, J=25.6 Hz, 2H), 4.47 (dd, J=8.0, 4.5 Hz, 10H), 3.62 (s, 2H), 3.22 (s, 3H), 2.65 (d, J=14.9 Hz, 2H), 2.47-2.35 (m, 1H), 1.97-1.82 (m, 2H), 1.80-1.67 (m, 2H), 1.58 (brs, 2H), 0.96 (t, J=7.4 Hz, 3H). Mass (m/z): 434.2 [M+H]⁺.

### Compound 69

### 7-((6-(2,6-dimethylmorpholino)-2-methylpyridin-3-yl)amino)-4-methyl-2H-benzo[b][1,4]oxazin-3(4H)-one

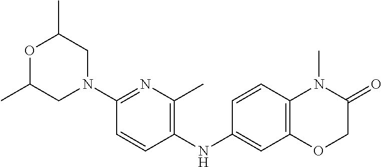 [SMILES: Cc1nc(N2CC(C)OC(C)C2)ccc1Nc1ccc2c(c1)OCC(=O)N2C]

The title compound 69 (19.1 mg) was prepared in a yield of 9% as a light yellow solid from 7-bromo-4-methyl-2H-benzo[b][1,4]oxazin-3(4H)-one (143 mg, 0.60 mmol), 6-(2,6-dimethylmorpholino)-2-methylpyridin-3-amine (120 mg, 0.54 mmol), 1,4-dioxane (2 mL), Pd₂(dba)₃ (50 mg, 0.05 mmol), Xphos (51 mg, 0.11 mmol) and Cs₂CO₃ (354 mg, 1.09 mmol) according to the procedure for compound 1. ¹H NMR (400 MHz, MeOD) δ 7.35 (d, J=8.7 Hz, 1H), 6.92 (d, J=8.7 Hz, 1H), 6.63 (d, J=8.8 Hz, 1H), 6.32 (dd, J=8.6, 2.5 Hz, 1H), 6.24 (d, J=2.5 Hz, 1H), 4.51 (s, 2H), 4.05 (dt, J=11.5, 2.0 Hz, 2H), 3.79-3.66 (m, 2H), 3.30 (s, 3H), 2.46-2.35 (m, 2H), 2.28 (s, 3H), 1.23 (d, J=6.2 Hz, 6H). Mass (m/z): 383.4 [M+H]⁺.

### Compound 70

### 7-((4-(2,6-dimethylmorpholino)-3-methoxyphenyl)amino)-4-methyl-2H-benzo[b][1,4]oxazin-3(4H)-one

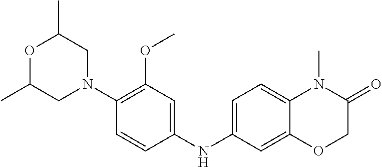 [SMILES: COc1cc(Nc2ccc3c(c2)OCC(=O)N3C)ccc1N1CC(C)OC(C)C1]

The title compound 70 (20.8 mg) was prepared in a yield of 15.5% as a pink solid from 4-(2,6-dimethylmorpholino)-3-methoxyaniline (80 mg, 0.339 mmol), 7-bromo-4-methyl-2H-benzo[b][1,4]oxazin-3(4H)-one (81.7 mg, 0.339 mmol), dioxane (15 mL), Pd₂(dba)₃ (15.5 mg, 0.017 mmol), X-phos (16.2 mg, 0.034 mmol) and Cs₂CO₃ (166 mg, 0.509 mmol) according to the procedure for compound 1.1H NMR (400 MHz, DMSO) δ 7.95 (s, 1H), 7.00 (d, J=8.7 Hz, 1H), 6.78 (d, J=8.4 Hz, 1H), 6.71 (dd, J=8.7, 2.4 Hz, 1H), 6.64-6.55 (m, 3H), 4.58 (s, 2H), 3.73 (s, 3H), 3.72-3.65 (m, 2H), 3.23 (s, 3H), 3.13 (d, J=10.6 Hz, 2H), 2.20 (t, J=10.8 Hz, 2H), 1.09 (d, J=6.2 Hz, 6H). Mass (m/z): 398.5 [M+H]⁺.

### Compound 71

### 7-((6-(2,6-dimethylmorpholino)naphthalen-2-yl)amino)-4-methyl-2H-benzo[b][1,4]oxazin-3(4H)-one

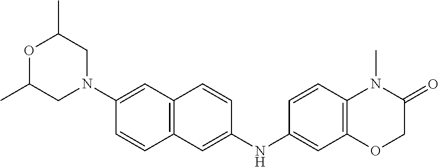 [SMILES: CC1CN(c2ccc3cc(Nc4ccc5c(c4)OCC(=O)N5C)ccc3c2)CC(C)O1]

The title compound 71 (45.8 mg) was prepared in a yield of 28% as a grey solid from 7-bromo-4-methyl-2H-benzo[b][1,4]oxazin-3(4H)-one (112 mg, 0.47 mmol), 6-(2,6-dimethylmorpholino)naphthalen-2-amine (100 mg, 0.39 mmol), 1,4-dioxane (2 mL), Pd₂(dba)₃ (36 mg, 0.04 mmol), Xphos (37 mg, 0.08 mmol) and Cs₂CO₃ (255 mg, 0.78 mmol) according to the procedure for compound 1. ¹H NMR (400 MHz, DMSO) δ 8.20 (s, 1H), 7.61 (dd, J=19.6, 9.0 Hz, 2H), 7.34 (d, J=2.2 Hz, 1H), 7.29 (dd, J=9.1, 2.5 Hz, 1H), 7.17 (dd, J=8.8, 2.3 Hz, 1H), 7.10-7.02 (m, 2H), 6.83 (dd, J=8.7, 2.5 Hz, 1H), 6.74 (d, J=2.4 Hz, 1H), 4.61 (s, 2H), 3.81-3.69 (m, 2H), 3.67-3.59 (m, 2H), 3.25 (s, 3H), 2.33-2.23 (m, 2H), 1.18 (d, J=6.2 Hz, 6H). Mass (m/z): 418.4 [M+H]⁺.

### Compound 72

### 4-methyl-7-((6-(4-(trifluoromethyl)piperidin-1-yl)naphthalen-2-yl)amino)-2H-benzo[b][1,4]oxazin-3(4H)-one

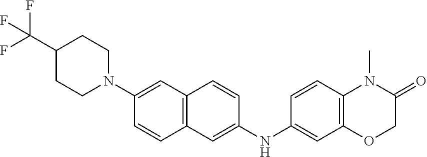 [SMILES: CN1C(=O)COc2cc(Nc3ccc4cc(N5CCC(C(F)(F)F)CC5)ccc4c3)ccc21]

The title compound 72 (44.7 mg) was prepared in a yield of 29.5% as an off-white solid from 7-bromo-4-methyl-2H-benzo[b][1,4]oxazin-3(4H)-one (97 mg, 0.41 mmol), 6-(2,6-dimethylmorpholino) naphthalen-2-amine (100 mg, 0.34 mmol), 1,4-dioxane (2 mL), Pd₂(dba)₃ (31 mg, 0.03 mmol), Xphos (32 mg, 0.07 mmol) and Cs₂CO₃ (221 mg, 0.68 mmol) according to the procedure for compound 1. ¹H NMR (400 MHz, DMSO-d₆) δ 8.20 (s, 1H), 7.63 (d, J=8.8 Hz, 1H), 7.58 (d, J=9.1 Hz, 1H), 7.34 (d, J=2.2 Hz, 1H), 7.27 (dd, J=9.1, 2.5 Hz, 1H), 7.17 (dd, J=8.8, 2.3 Hz, 1H), 7.12 (d, J=2.5 Hz, 1H), 7.06 (d, J=8.7 Hz, 1H), 6.83 (dd, J=8.7, 2.4 Hz, 1H), 6.74 (d, J=2.4 Hz, 1H), 4.61 (s, 2H), 3.83 (d, J=12.4 Hz, 2H), 3.26 (s, 3H), 2.79-2.68 (m, 2H), 2.54-2.51 (m, 1H), 1.97-1.88 (m, 2H), 1.69-1.54 (m, 2H). Mass (m/z): 446.4 [M+H]⁺.

### Compound 73

### 7-((4-(2,6-dimethylmorpholino)-3-fluorophenyl)amino)-4-methyl-2H-benzo[b][1,4]oxazin-3(4H)-one

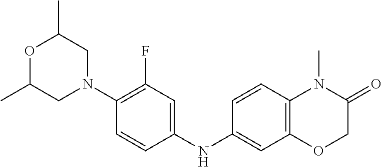 [SMILES: CC1CN(c2ccc(Nc3ccc4c(c3)OCC(=O)N4C)cc2F)CC(C)O1]

The title compound 73 (66.1 mg) was prepared in a yield of 32% as a brown solid from 7-bromo-4-methyl-2H-benzo[b][1,4]oxazin-3(4H)-one (154 mg, 0.64 mmol), 4-(2,6-dimethylmorpholino)-3-fluoroaniline (120 mg, 0.54 mmol), 1,4-dioxane (2 mL), Pd₂(dba)₃ (49 mg, 0.05 mmol), Xphos (51 mg, 0.11 mmol) and Cs₂CO₃ (349 mg, 1.07 mmol) according to the procedure for compound 1. ¹H NMR (400 MHz, DMSO) δ 8.10 (s, 1H), 7.03 (d, J=8.7 Hz, 1H), 6.93 (dd, J=9.9, 8.4 Hz, 1H), 6.83-6.75 (m, 2H), 6.73 (dd, J=8.6, 2.5 Hz, 1H), 6.63 (d, J=2.4 Hz, 1H), 4.60 (s, 2H), 3.79-3.66 (m, 2H), 3.24 (s, 3H), 3.13-3.05 (m, 2H), 2.35-2.25 (m, 2H), 1.11 (d, J=6.2 Hz, 6H). Mass (m/z): 386.4 [M+H]⁺.

### Compound 74

### 7-((4-(2,6-dimethylmorpholino)-2-fluorophenyl)amino)-4-methyl-2H-benzo[b][1,4]oxazin-3(4H)-one

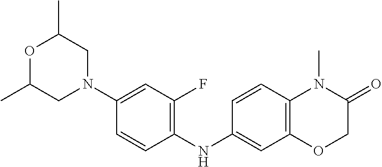 [SMILES: CC1CN(c2ccc(Nc3ccc4c(c3)OCC(=O)N4C)c(F)c2)CC(C)O1]

The title compound 74 (97.2 mg) was prepared in a yield of 43% as an off-white solid from 7-bromo-4-methyl-2H-benzo[b][1,4] oxazin-3(4H)-one (139 mg, 0.58 mmol), 6-(2,6-dimethylmorpholino) naphthalen-2-amine (130 mg, 0.58 mmol), 1,4-dioxane (2 mL), Pd₂(dba)₃ (53 mg, 0.06 mmol), Xphos (55 mg, 0.12 mmol) and Cs₂CO₃ (378 mg, 1.16 mmol) according to the procedure for compound 1. ¹H NMR (400 MHz, DMSO) δ 7.53 (s, 1H), 7.11 (t, J=9.3 Hz, 1H), 6.94 (d, J=8.7 Hz, 1H), 6.85 (dd, J=14.3, 2.7 Hz, 1H), 6.72 (dd, J=9.0, 2.7 Hz, 1H), 6.44 (dd, J=8.6, 2.5 Hz, 1H), 6.36 (d, J=2.4 Hz, 1H), 4.55 (s, 2H), 3.73-3.61 (m, 2H), 3.59-3.50 (m, 2H), 3.21 (s, 3H), 2.28-2.18 (m, 2H), 1.15 (d, J=6.2 Hz, 6H). Mass (m/z): 386.4 [M+H]⁺.

### Compound 75

### 4-methyl-7-((4-(4-(trifluoromethyl)piperidin-1-yl)phenyl)amino)-2H-pyrido[3,2-b][1,4]oxazin-3(4H)-one

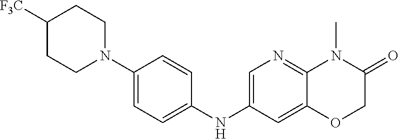 [SMILES: CN1C(=O)COc2cc(Nc3ccc(N4CCC(C(F)(F)F)CC4)cc3)cnc21]

The title compound 75 (50.3 mg) was prepared in a yield of 20.1% as a white solid from 7-bromo-4-methyl-2H-pyrido[3,2-b][1,4] oxazin-3(4H)-one (150 mg, 0.62 mmol), 4-(4-(trifluoromethyl)piperidin-1-yl)aniline (166 mg, 0.68 mmol) in 1,4-dioxane (5 mL), Pd₂(dba)₃ (28 mg, 0.03 mmol), X-phos (29.4 mg, 0.06 mmol) and Cs₂CO₃ (402 mg, 1.23 mmol) according to the procedure for compound 1. ¹H NMR (400 MHz, DMSO-d₆) δ 7.96 (s, 1H), 7.71 (d, J=2.3 Hz, 1H), 7.01-6.95 (m, 2H), 6.95-6.87 (m, 3H), 4.69 (s, 2H), 3.63 (d, J=12.3 Hz, 2H), 3.28 (s, 3H), 2.63 (td, J=12.2, 2.4 Hz, 2H), 2.42 (td, J=8.6, 4.1 Hz, 1H), 1.93-1.83 (m, 2H), 1.57 (qd, J=12.5, 4.1 Hz, 2H). LC-MS (m/z) 407.2 [M+H]⁺.

### Compound 76

### 7-((4-(2,6-dimethylmorpholino)phenyl)amino)-4-methyl-2H-pyrido[3,2-b][1,4]oxazin-3(4H)-one

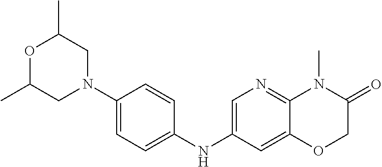 [SMILES: CC1CN(c2ccc(Nc3cnc4c(c3)OCC(=O)N4C)cc2)CC(C)O1]

The title compound 76 (100 mg) was prepared in a yield of 44% as a light yellow solid from 7-bromo-4-methyl-2H-pyrido[3,2-b][1,4]oxazin-3(4H)-one (150 mg, 0.62 mmol), 4-(2,6-dimethylmorpholino) aniline (140 mg, 0.68 mmol), 1,4-dioxane (5 mL), Pd₂(dba)₃ (28 mg, 0.03 mmol), X-phos (29.4 mg, 0.06 mmol) and Cs₂CO₃ (402 mg, 1.23 mmol) according to the procedure for compound 1. ¹H NMR (400 MHz, DMSO-d₆) δ 7.94 (s, 1H), 7.70 (d, J=2.4 Hz, 1H), 6.99 (d, J=2.2 Hz, 1H), 6.98-6.84 (m, 4H), 4.69 (s, 2H), 3.69 (dqd, J=12.4, 6.1, 2.1 Hz, 2H), 3.45 (dt, J=10.9, 2.1 Hz, 2H), 3.34 (s, 3H), 2.19 (dd, J=11.8, 10.2 Hz, 2H), 1.24-1.11 (m, 6H). LC-MS (m/z) 369.2 [M+H]⁺.

### Compound 77

### 7-((3-fluoro-4-(4-(trifluoromethyl)piperidin-1-yl)phenyl)amino)-4-methyl-2H-benzo[b][1,4]oxazin-3(4H)-one

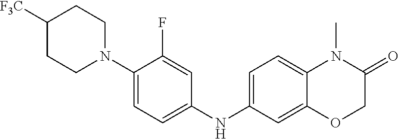 [SMILES: CN1C(=O)COc2cc(Nc3ccc(N4CCC(C(F)(F)F)CC4)c(F)c3)ccc21]

The title compound 77 (13 mg) was prepared in a yield of 44% as a white solid from 7-bromo-4-methyl-2H-pyrido[3,2-b][1,4]oxazin-3(4H)-one (120 mg, 0.49 mmol), 3-fluoro-4-(4-(trifluoromethyl)piperidin-1-yl) aniline (130 mg, 0.49 mmol), 1,4-dioxane (5 mL), Pd₂(dba)₃ (22.7 mg, 0.025 mmol), X-phos (23.6 mg, 0.049 mmol) and Cs₂CO₃ (323.2 mg, 0.991 mmol) according to the procedure for compound 1. ¹H NMR (400 MHz, DMSO-d₆) δ 8.11 (s, 1H), 7.03 (d, J=8.7 Hz, 1H), 6.97 (dd, J=10.0, 8.4 Hz, 1H), 6.79 (s, 1H), 6.77 (t, J=2.7 Hz, 1H), 6.73 (dd, J=8.6, 2.5 Hz, 1H), 6.64 (d, J=2.5 Hz, 1H), 4.60 (s, 2H), 3.28 (d, J=11.7 Hz, 2H), 3.24 (s, 3H), 2.65 (dd, J=12.6, 10.2 Hz, 2H), 2.46-2.36 (m, 1H), 1.93-1.84 (m, 2H), 1.60 (qd, J=12.4, 4.0 Hz, 2H). LC-MS (m/z) 424.2 [M+H]⁺.

### Compound 78

### 4-methyl-7-((2-methyl-4-(4-(trifluoromethyl)piperidin-1-yl)phenyl) amino)-2H-benzo[b][1,4]oxazin-3(4H)-one

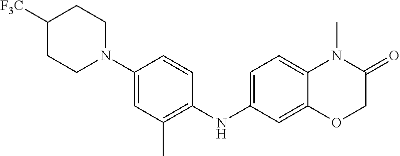 [SMILES: Cc1cc(N2CCC(C(F)(F)F)CC2)ccc1Nc1ccc2c(c1)OCC(=O)N2C]

The title compound 78 (32 mg) was prepared in a yield of 15.4% as a white solid from 7-bromo-4-methyl-2H-pyrido[3,2-b][1,4]oxazin-3(4H)-one (120 mg, 0.49 mmol), 2-methyl-4-(4-(trifluoromethyl) piperidin-1-yl)aniline (130 mg, 0.49 mmol), 1,4-dioxane (5 mL), Pd₂(dba)₃ (22.7 mg, 0.025 mmol), X-phos (23.6 mg, 0.049 mmol) and Cs₂CO₃ (323.2 mg, 0.991 mmol) according to the procedure for compound 1. ¹H NMR (400 MHz, DMSO-d₆) δ 7.20 (s, 1H), 6.98 (d, J=8.6 Hz, 1H), 6.91 (d, J=8.7 Hz, 1H), 6.85 (d, J=2.8 Hz, 1H), 6.76 (dd, J=8.7, 2.9 Hz, 1H), 6.35 (dd, J=8.7, 2.5 Hz, 1H), 6.27 (d, J=2.4 Hz, 1H), 4.53 (s, 2H), 3.70 (d, J=12.1 Hz, 2H), 3.20 (s, 3H), 2.65 (td, J=12.3, 2.5 Hz, 2H), 2.48-2.41 (m, 1H), 2.12 (s, 3H), 1.92-1.83 (m, 2H), 1.56 (qd, J=12.5, 4.1 Hz, 2H). LC-MS (m/z) 420.2 [M+H]⁺.

### Compound 79

### 7-((2,6-dimethyl-4-(4-(trifluoromethyl)piperidin-1-yl)phenyl)amino)-4-methyl-2H-benzo[b][1,4]oxazin-3(4H)-one

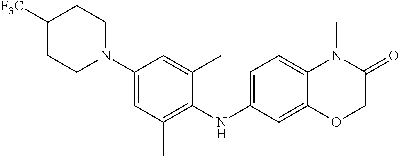 [SMILES: Cc1cc(N2CCC(C(F)(F)F)CC2)cc(C)c1Nc1ccc2c(c1)OCC(=O)N2C]

The title compound 79 (38 mg) was prepared in a yield of 16.3% as a white solid from 7-bromo-4-methyl-2H-pyrido[3,2-b][1,4]oxazin-3(4H)-one (130 mg, 0.54 mmol), 2,6-dimethyl-4-(4-(trifluoromethyl) piperidin-1-yl)aniline (146 mg, 0.54 mmol), 1,4-dioxane (5 mL), Pd₂(dba)₃ (24.6 mg, 0.027 mmol), X-phos (25.6 mg, 0.054 mmol) and Cs₂CO₃ (350 mg, 1.07 mmol) according to the procedure for compound 1. ¹H NMR (400 MHz, DMSO-d₆) δ 7.06 (s, 1H), 6.86 (d, J=8.7 Hz, 1H), 6.72 (s, 2H), 6.05 (dd, J=8.6, 2.5 Hz, 1H), 5.98 (d, J=2.4 Hz, 1H), 4.51 (s, 2H), 3.75 (d, J=12.3 Hz, 2H), 3.18 (s, 3H), 2.67 (td, J=12.0, 2.4 Hz, 2H), 2.47-2.39 (m, 1H), 2.07 (d, J=2.8 Hz, 6H), 1.92-1.83 (m, 2H), 1.55 (qd, J=12.6, 4.1 Hz, 2H). LC-MS (m/z) 434.2 [M+H]⁺.

### Compound 80

### 4-(2-(dimethylamino)ethyl)-7-((4-(2,6-dimethylmorpholino)phenyl)amino)-2H-benzo[b][1,4]oxazin-3(4H)-one

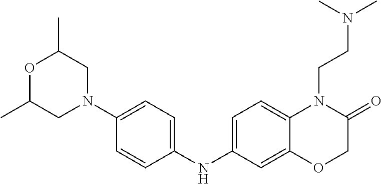 [SMILES: CC1CN(c2ccc(Nc3ccc4c(c3)OCC(=O)N4CCN(C)C)cc2)CC(C)O1]

The title compound 80 (35.2 mg) was prepared in a yield of 24.4% as a pink solid from 4-(2,6-dimethylmorpholino)aniline (70 mg, 0.339 mmol), 7-bromo-4-(2-(dimethylamino)ethyl)-2H-benzo[b][1,4]oxazin-3(4H)-one (101 mg, 0.339 mmol), dioxane (5 mL), Pd₂(dba)₃ (15.6 mg, 0.017 mmol), X-phos (16.2 mg, 0.034 mmol) and Cs₂CO₃ (166 mg, 0.509 mmol) according to the procedure for compound 1. ¹H NMR (400 MHz, DMSO) δ 7.81 (s, 1H), 6.99 (dd, J=15.6, 8.9 Hz, 3H), 6.88 (d, J=9.0 Hz, 2H), 6.61 (dd, J=8.7, 2.4 Hz, 1H), 6.53 (d, J=2.4 Hz, 1H), 4.54 (s, 2H), 3.93 (t, J=6.9 Hz, 2H), 3.68 (dd, J=11.5, 5.0 Hz, 2H), 3.44 (d, J=10.6 Hz, 2H), 2.39 (t, J=6.9 Hz, 2H), 2.29 (dd, J=19.8, 10.7 Hz, 1H), 2.23-2.13 (m, 7H), 1.15 (t, J=5.7 Hz, 6H). Mass (m/z): 425.6 [M+H]⁺

### Compound 81

### 7-((4-(2,6-dimethylmorpholino)phenyl)amino)-2-ethyl-4-methyl-2H-benzo[b][1,4]oxazin-3(4H)-one

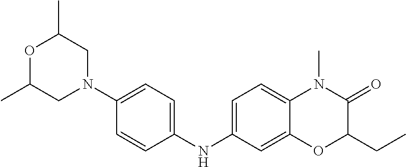 [SMILES: CCC1Oc2cc(Nc3ccc(N4CC(C)OC(C)C4)cc3)ccc2N(C)C1=O]

The title compound 81 (12.5 mg) was prepared in a yield of 17% as a pink solid from 4-(2,6-dimethylmorpholino)aniline (38.3 mg, 0.186 mmol), 7-bromo-2-ethyl-4-methyl-2H-benzo[b][1,4]oxazin-3(4H)-one (50 mg, 0.186 mmol), dioxane (5 mL), Pd₂(dba)₃ (8.52 mg, 0.0093 mmol), X-phos (8.9 mg, 0.0186 mmol) and Cs₂CO₃ (91 mg, 0.279 mmol) according to the procedure for compound 1. ¹H NMR (400 MHz, DMSO-d₆) δ 7.79 (s, 1H), 7.00-6.85 (m, 4H), 6.63-6.49 (m, 2H), 4.46 (dd, J=8.0, 4.6 Hz, 1H), 3.76-3.61 (m, 2H), 3.45 (d, J=10.8 Hz, 2H), 3.22 (s, 3H), 2.25-2.13 (m, 2H), 1.15 (t, J=5.7 Hz, 5H), 1.02-0.85 (m, 3H). Mass (m/z): 396.5 [M+H]⁺

### Compound 82

### 7-((4-(tert-butyl)phenyl)amino)-4-(2-hydroxyethyl)-2H-benzo[b][1,4]oxazin-3(4H)-one

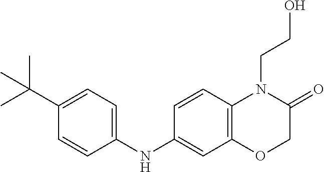 [SMILES: CC(C)(C)c1ccc(Nc2ccc3c(c2)OCC(=O)N3CCO)cc1]

To a solution of 7-((4-(tert-butyl)phenyl)amino)-4-(2-((tetrahydro-2H-pyran-2-yl)oxy)ethyl)-2H-benzo[b][1,4]oxazin-3(4H)-one (100 mg, 0.24 mmol) in methanol (3 mL) was added 4-methylbenzenesulfonic acid (44 mg, 0.26 mmol). The mixture was stirred at r.t. for 1 h, the mixture was concentrated and purified by prep HPLC to give 7-((4-(tert-butyl)phenyl)amino)-4-(2-hydroxyethyl)-2H-benzo[b][1,4]oxazin-3(4H)-one (16.9 mg, 43%) as a light yellow solid. Mass (m/z): 341.4 [M+H]⁺. ¹H NMR (400 MHz, DMSO-d₆) δ 7.98 (s, 1H), 7.26-7.17 (m, 2H), 7.08 (d, J=8.8 Hz, 1H), 6.98-6.89 (m, 2H), 6.68 (dd, J=8.7, 2.5 Hz, 1H), 6.60 (d, J=2.5 Hz, 1H), 4.83 (t, J=5.7 Hz, 1H), 4.53 (s, 2H), 3.87 (t, J=6.3 Hz, 2H), 3.53 (q, J=6.1 Hz, 2H), 1.22 (s, 9H).

### Compound 83

### 4-(2-hydroxyethyl)-7-((4-(4-(trifluoromethyl)piperidin-1-yl)phenyl)amino)-2H-benzo[b][1,4]oxazin-3(4H-one

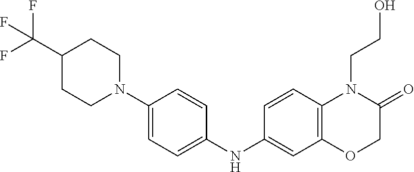 [SMILES: O=C1COc2cc(Nc3ccc(N4CCC(C(F)(F)F)CC4)cc3)ccc2N1CCO]

To a solution of 4-(2-((tetrahydro-2H-pyran-2-yl)oxy)ethyl)-7-((4-(4-(trifluoromethyl)piperidin-1-yl)phenyl)amino)-2H-benzo[b][1,4]oxazin-3(4H)-one (100 mg, 0.19 mmol) in methanol (3 mL) was added 4-methylbenzenesulfonic acid (36 mg, 0.21 mmol). The mixture was stirred at r.t. for 1 h, concentrated and purified by prep HPLC to give 4-(2-hydroxyethyl)-7-((4-(4-(trifluoromethyl)piperidin-1-yl)phenyl)amino)-2H-benzo[b][1,4]oxazin-3(4H)-one (16.9 mg, 43%) as a grey solid. ¹H NMR (400 MHz, DMSO) δ 7.81 (s, 1H), 7.08 (d, J=8.8 Hz, 1H), 6.96 (d, J=9.0 Hz, 2H), 6.96-6.86 (m, 2H), 6.60 (dd, J=8.7, 2.5 Hz, 1H), 6.53 (d, J=2.5 Hz, 1H), 4.86 (t, J=5.7 Hz, 1H), 4.54 (s, 2H), 3.89 (t, J=6.3 Hz, 2H), 3.64 (s, 1H), 3.62-3.51 (m, 3H), 2.68-2.57 (m, 2H), 2.47-2.36 (m, 1H), 1.93-1.83 (m, 2H), 1.64-1.49 (m, 2H). Mass (m/z): 436.4 [M+H]⁺.

### Compound 84

### 7-((4-(2,6-dimethylmorpholino)phenyl)amino)-4-(2-hydroxyethyl)-2H-benzo[b][1,4]oxazin-3(4H)-one

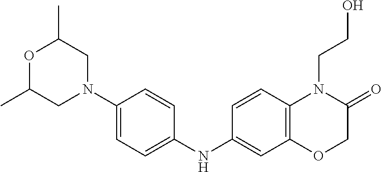 [SMILES: CC1CN(c2ccc(Nc3ccc4c(c3)OCC(=O)N4CCO)cc2)CC(C)O1]

To a solution of 4-(2-hydroxyethyl)-7-((4-(4-(trifluoromethyl) piperidin-1-yl)phenyl)amino)-2H-benzo[b][1,4]oxazin-3(4H)-one (100 mg, 0.21 mmol) in methanol (3 mL) was added 4-methylbenzenesulfonic acid (39 mg, 0.23 mmol). The mixture was stirred at r.t. for 1 h, the mixture was concentrated and purified by prep HPLC to give 7-((4-(2,6-dimethylmorpholino)phenyl)amino)-4-(2-hydroxyethyl)-2H-ben zo[b][1,4]oxazin-3(4H)-one (16.9 mg, 43%) as a grey solid. ¹H NMR (400 MHz, DMSO) δ 7.75 (s, 1H), 7.05 (d, J=8.8 Hz, 1H), 6.94 (d, J=8.9 Hz, 2H), 6.90-6.82 (m, 2H), 6.57 (dd, J=8.7, 2.5 Hz, 1H), 6.50 (d, J=2.4 Hz, 1H), 4.82 (s, 1H), 4.51 (s, 2H), 3.87 (t, J=6.3 Hz, 2H), 3.73-3.61 (m, 2H), 3.53 (t, J=6.4 Hz, 2H), 3.46-3.38 (m, 2H), 2.17 (t, J=11.0 Hz, 2H), 1.12 (d, J=6.2 Hz, 6H). Mass (m/z): 398.4 [M+H]⁺.

### Compound 85

### 7-((4-(4-(trifluoromethyl)piperidin-1-yl)phenyl)amino)-2H-benzo[b][1,4]oxazin-3(4H)-one

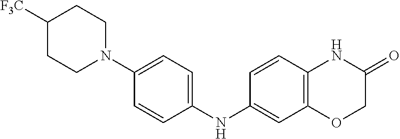 [SMILES: O=C1COc2cc(Nc3ccc(N4CCC(C(F)(F)F)CC4)cc3)ccc2N1]

The title compound 85 (8.6 mg) was prepared in a total yield of 10.0% as a yellow solid from 4-(4-(trifluoromethyl)piperidin-1-yl)aniline (59 mg, 0.241 mmol), 7-bromo-2H-benzo[b][1,4]oxazin-3(4H)-one (50 mg, 0.219 mmol), Pd₂(dba)₃ (2 mg, 0.002 mmol), X-phos (6 mg, 0.01 mmol) and Cs₂CO₃ (108 mg, 0.329 mmol) and 1,4-dioxane (5 mL) according to the procedure for compound 1. ¹H NMR (400 MHz, DMSO-d₆) δ 10.46 (s, 1H), 7.71 (s, 1H), 6.96-6.84 (m, 4H), 6.71 (d, J=8.4 Hz, 1H), 6.57-6.49 (m, 2H), 4.48 (s, 2H), 3.61 (d, J=12.4 Hz, 2H), 2.61 (td, J=12.4, 2.4 Hz, 2H), 2.42 (ddt, J=12.4, 8.8, 4.0 Hz, 1H), 1.91-1.82 (m, 2H), 1.57 (qd, J=12.4, 4.0 Hz, 2H). Mass (m/z): 392.3 [M+H]⁺.

### Compound 86

### 4-methyl-7-((3-methyl-4-(4-(trifluoromethyl)piperidin-1-yl)phenyl)amino)-2H-benzo[b][1,4]oxazin-3(4H)-one

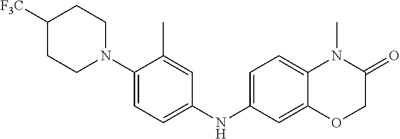 [SMILES: Cc1cc(Nc2ccc3c(c2)OCC(=O)N3C)ccc1N1CCC(C(F)(F)F)CC1]

The title compound 86 (156 mg) was prepared in a yield of 60% as a light yellow solid from 7-bromo-4-methyl-2H-pyrido[3,2-b][1,4]oxazin-3(4H)-one (150 mg, 0.62 mmol), 3-methyl-4-(4-(trifluoromethyl) piperidin-1-yl)aniline (160 mg, 0.62 mmol), 1,4-dioxane (5 mL), Pd₂(dba)₃ (28.3 mg, 0.03 mmol), X-phos (29.5 mg, 0.06 mmol) and Cs₂CO₃ (404 mg, 1.24 mmol) according to the procedure for compound 1. ¹H NMR (400 MHz, DMSO-d₆) δ 7.89 (s, 1H), 6.99 (d, J=8.7 Hz, 1H), 6.94 (d, J=8.3 Hz, 1H), 6.89-6.81 (m, 2H), 6.69 (dd, J=8.7, 2.5 Hz, 1H), 6.60 (d, J=2.4 Hz, 1H), 4.58 (s, 2H), 3.23 (s, 3H), 3.04 (d, J=11.4 Hz, 2H), 2.61 (td, J=12.0, 2.3 Hz, 2H), 2.45-2.36 (m, 1H), 2.19 (s, 3H), 1.93-1.84 (m, 2H), 1.60 (qd, J=12.3, 4.0 Hz, 2H). LC-MS (m/z) 420.2 [M+H]⁺.

### Compound 87

### 7-((4-(2,6-dimethylmorpholino)phenyl)amino)-5-fluoro-4-methyl-2H-benzo[b][1,4]oxazin-3(4H)-one

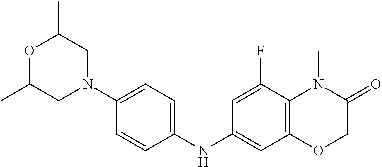 [SMILES: CC1CN(c2ccc(Nc3cc(F)c4c(c3)OCC(=O)N4C)cc2)CC(C)O1]

The title compound 87 (39.4 mg) was prepared in a yield of 22.2% as a white solid from 7-bromo-5-fluoro-4-methyl-2H-benzo[b][1,4]oxazin-3(4H)-one (120 mg, 0.46 mmol), 4-(2,6-dimethylmorpholino) aniline (95 mg, 0.46 mmol), 1,4-dioxane (5 mL), Pd₂(dba)₃ (21 mg, 0.023 mmol), X-phos (22 mg, 0.046 mmol) and Cs₂CO₃ (301 mg, 0.92 mmol) according to the procedure for compound 1. ¹H NMR (400 MHz, DMSO-d₆) δ 8.06 (s, 1H), 7.04-6.96 (m, 2H), 6.96-6.87 (m, 2H), 6.44-6.32 (m, 2H), 4.54 (s, 2H), 3.72-3.64 (m, 2H), 3.48 (dt, J=11.0, 2.1 Hz, 2H), 3.29 (d, J=5.5 Hz, 3H), 2.20 (dd, J=11.8, 10.2 Hz, 2H), 1.14 (d, J=6.2 Hz, 6H). LC-MS (m/z) 386.2 [M+H]⁺.

### Compound 88

### 5-fluoro-4-methyl-7-((4-(4-(trifluoromethyl)piperidin-1-yl)phenyl)amino)-2H-benzo[b][1,4]oxazin-3(4H)-one

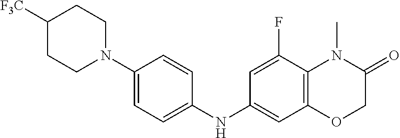 [SMILES: CN1C(=O)COc2cc(Nc3ccc(N4CCC(C(F)(F)F)CC4)cc3)cc(F)c21]

The titled compound 88 (46.5 mg, 23.8%) as an off-white solid was prepared according to the procedure for compound 1. ¹H NMR (400 MHz, DMSO-d₆) δ 8.08 (s, 1H), 7.00 (d, J=6.5 Hz, 1H), 6.98-6.90 (m, 3H), 6.41 (dd, J=15.1, 2.5 Hz, 1H), 6.36 (dd, J=2.5, 1.2 Hz, 1H), 4.54 (s, 2H), 3.67 (d, J=12.3 Hz, 2H), 3.29 (d, J=5.5 Hz, 3H), 2.63 (dd, J=12.3, 2.5 Hz, 2H), 2.46-2.36 (m, 1H), 1.93-1.84 (m, 2H), 1.56 (qd, J=12.5, 4.1 Hz, 2H). LC-MS (m/z) 424.2 [M+H]⁺.

### Compound 89

### 7-((4-(8-oxa-3-azabicyclo[3.2.1]octan-3-yl)-3-fluorophenyl)amino)-4-methyl-2H-benzo[b][1,4]oxazin-3(4H)-one

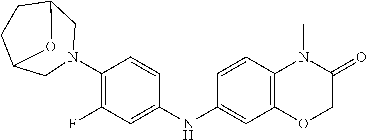 [SMILES: CN1C(=O)COc2cc(Nc3ccc(N4CC5CCC(C4)O5)c(F)c3)ccc21]

The title compound 89 (51.1 mg) was prepared in a yield of 26.9% as a light yellow solid from 7-bromo-4-methyl-2H-benzo[b][1,4]oxazin-3(4H)-one (120 mg, 0.49 mmol), 4-(2,6-dimethylmorpholino) aniline (110 mg, 0.49 mmol), 1,4-dioxane (5 mL), Pd₂(dba)₃ (22.7 mg, 0.025 mmol), X-phos (23.6 mg, 0.049 mmol) and Cs₂CO₃ (323 mg, 0.99 mmol) according to the procedure for compound 1. ¹H NMR (400 MHz, DMSO-d₆) δ 8.07 (s, 1H), 7.02 (d, J=8.7 Hz, 1H), 6.88 (dd, J=10.0, 8.4 Hz, 1H), 6.82-6.66 (m, 3H), 6.62 (d, J=2.5 Hz, 1H), 4.59 (s, 2H), 4.32 (dt, J=4.4, 2.2 Hz, 2H), 3.23 (s, 3H), 2.94 (dd, J=10.6, 1.5 Hz, 2H), 2.86 (dd, J=11.4, 2.0 Hz, 2H), 1.95 (q, J=6.2, 5.6 Hz, 2H), 1.81 (dd, J=7.8, 4.2 Hz, 2H). LC-MS (m/z) 384.2 [M+H]⁺.

### Compound 90

### 7-((2-methoxy-4-(4-(trifluoromethyl)piperidin-1-yl)phenyl)amino)-4-methyl-2H-benzo[b][1,4]oxazin-3(4H)-one

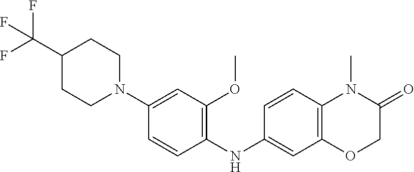 [SMILES: COc1cc(N2CCC(C(F)(F)F)CC2)ccc1Nc1ccc2c(c1)OCC(=O)N2C]

The title compound 90 (48 mg) was prepared in a yield of 37.8% as a gray solid from 2-methoxy-4-(4-(trifluoromethyl)piperidin-1-yl)aniline (80 mg, 0.292 mmol), 7-bromo-4-methyl-2H-benzo[b][1,4]oxazin-3(4H)-one (70.4 mg, 0.292 mmol), dioxane (15 mL), Pd₂(dba)₃ (13.4 mg, 0.015 mmol), X-phos (14 mg, 0.0292 mmol) and Cs₂CO₃ (143 mg, 0.438 mmol) according to the procedure for compound 1. ¹H NMR (400 MHz, DMSO) δ 7.10 (s, 1H), 7.01 (d, J=8.6 Hz, 1H), 6.91 (d, J=8.7 Hz, 1H), 6.65 (d, J=2.5 Hz, 1H), 6.55-6.39 (m, 3H), 4.53 (s, 2H), 3.78 (d, J=13.8 Hz, 3H), 3.72 (d, J=12.4 Hz, 2H), 3.21 (s, 3H), 2.67 (dd, J=12.2, 10.5 Hz, 2H), 2.44 (dd, J=11.7, 8.3 Hz, 1H), 1.89 (d, J=13.4 Hz, 2H), 1.58 (qd, J=12.5, 3.9 Hz, 2H). Mass (m/z): 436.5 [M+H]⁺

### Compound 91

### 7-((3-methoxy-4-(4-(trifluoromethyl)piperidin-1-yl)phenyl)amino)-4-methyl-2H-benzo[b][1,4]oxazin-3(4H)-one

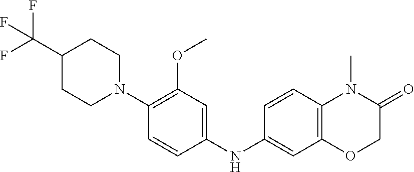 [SMILES: COc1cc(Nc2ccc3c(c2)OCC(=O)N3C)ccc1N1CCC(C(F)(F)F)CC1]

The title compound 91 (27.8 mg) was prepared in a yield of 21.9% as a gray solid from 3-methoxy-4-(4-(trifluoromethyl)piperidin-1-yl)aniline (80 mg, 0.292 mmol), 7-bromo-4-methyl-2H-benzo[b][1,4]oxazin-3(4H)-one (70.4 mg, 0.292 mmol), dioxane (15 mL), Pd₂(dba)₃ (13.4 mg, 0.015 mmol), X-phos (14 mg, 0.0292 mmol) and Cs₂CO₃ (143 mg, 0.438 mmol) according to the procedure for compound 1. ¹H NMR (400 MHz, DMSO-d₆) δ 7.93 (s, 1H), 6.96 (d, J=8.8 Hz, 1H), 6.77 (d, J=8.4 Hz, 1H), 6.68 (dd, J=8.7, 2.4 Hz, 1H), 6.62-6.47 (m, 3H), 4.54 (s, 2H), 3.73 (d, J=20.5 Hz, 3H), 3.26 (s, 2H), 3.19 (s, 3H), 2.51-2.48 (m, 2H), 1.82 (d, J=11.7 Hz, 2H), 1.55 (dt, J=13.1, 8.7 Hz, 2H). Mass (m/z): 436.5 [M+H]⁺

### Compound 92

### 7-((4-(2,6-dimethyltetrahydro-2H-pyran-4-yl)phenyl)amino)-2H-benzo[b][1,4]oxazin-3(4H)-one

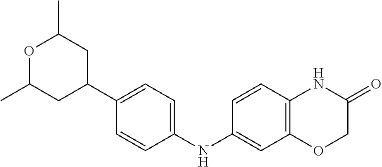 [SMILES: CC1CC(c2ccc(Nc3ccc4c(c3)OCC(=O)N4)cc2)CC(C)O1]

The title compound 92 (23 mg) was prepared in a yield of 11% as a white solid from 4-(2,6-dimethyltetrahydro-2H-pyran-4-yl)aniline (100 mg, 0.488 mmol), 7-bromo-2H-benzo[b][1,4]oxazin-3(4H)-one (122 mg, 0.536 mmol), t-BuOH (5 mL), Pd₂(dba)₃ (22.4 mg, 0.0244 mmol), Brettphos (26.2 mg, 0.0488 mmol) and Cs₂CO₃ (239 mg, 0.732 mmol) according to the procedure for compound 1. ¹H NMR (400 MHz, DMSO-d₆) δ 10.51 (s, 1H), 7.93 (s, 1H), 7.07 (d, J=8.5 Hz, 2H), 6.94 (t, J=10.7 Hz, 2H), 6.75 (d, J=8.4 Hz, 1H), 6.66-6.55 (m, 2H), 4.50 (s, 2H), 3.52 (dq, J=12.2, 6.1 Hz, 2H), 2.73-2.61 (m, 1H), 1.70 (d, J=15.8 Hz, 2H), 1.25-1.14 (m, 2H), 1.11 (t, J=12.2 Hz, 6H). Mass (m/z): 353.4 [M+H]⁺

### Compound 93

### 4-methyl-7-((4-(2,2,6,6-tetramethylmorpholino)phenyl)amino)-2H-benzo[b][1,4]oxazin-3(4H)-one

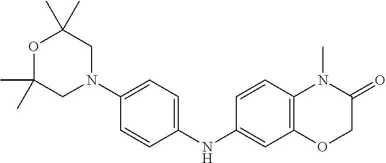 [SMILES: CN1C(=O)COc2cc(Nc3ccc(N4CC(C)(C)OC(C)(C)C4)cc3)ccc21]

The title compound 93 (32 mg) was prepared in a yield of 31% as an olive solid from 7-bromo-4-methyl-2H-benzo[b][1,4]oxazin-3(4H)-one (68 mg, 0.28 mmol), 4-(2,2,6,6-tetramethylmorpholino)aniline (60 mg, 0.26 mmol), 1,4-dioxane (2 mL), Pd₂(dba)₃ (23 mg, 0.03 mmol), Xphos (24 mg, 0.05 mmol) and Cs₂CO₃ (167 mg, 0.51 mmol) according to the procedure for compound 1. ¹H NMR (400 MHz, DMSO-d₆) δ 7.81 (s, 1H), 7.02-6.92 (m, 3H), 6.91-6.82 (m, 2H), 6.62 (dd, J=8.7, 2.5 Hz, 1H), 6.53 (d, J=2.4 Hz, 1H), 4.56 (s, 2H), 3.22 (s, 3H), 2.84 (s, 4H), 1.22 (s, 12H). Mass (m/z): 396.4 [M+H]⁺.

### Compound 94

### 4-(2-(dimethylamino)ethyl)-7-((4-(4-(trifluoromethyl)piperidin-1-yl) phenyl)amino)-2H-benzo[b][1,4]oxazin-3(4H)-one

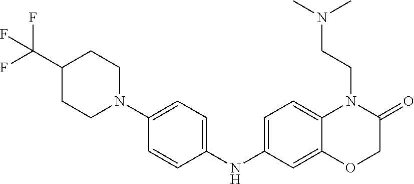 [SMILES: CN(C)CCN1C(=O)COc2cc(Nc3ccc(N4CCC(C(F)(F)F)CC4)cc3)ccc21]

The title compound 94 (36 mg) was prepared in a yield of 31% as a light pink solid from 7-bromo-4-(2-(dimethylamino)ethyl)-2H-benzo[b][1,4]oxazin-3(4H)-one (100 mg, 0.36 mmol), 4-(4-(trifluoromethyl) piperidin-1-yl)aniline (80 mg, 0.32 mmol), 1,4-dioxane (2 mL), Pd₂(dba)₃ (30 mg, 0.03 mmol), Xphos (31 mg, 0.07 mmol) and Cs₂CO₃ (213 mg, 0.66 mmol) according to the procedure for compound 1. ¹H NMR (400 MHz, DMSO-d₆) δ 7.83 (s, 1H), 7.01 (d, J=8.8 Hz, 1H), 7.00-6.93 (m, 2H), 6.93-6.86 (m, 2H), 6.62 (dd, J=8.7, 2.5 Hz, 1H), 6.54 (d, J=2.5 Hz, 1H), 4.54 (s, 2H), 3.93 (t, J=7.0 Hz, 2H), 3.66-3.59 (m, 2H), 2.68-2.57 (m, 2H), 2.46-2.35 (m, 3H), 2.18 (s, 6H), 1.92-1.83 (m, 2H), 1.64-1.49 (m, 2H).

### Compound 95

### 7-((4-(2,6-dimethylmorpholino)phenyl)amino)-4-(2-methoxyethyl)-2H-benzo[b][1,4]oxazin-3(4H)-one

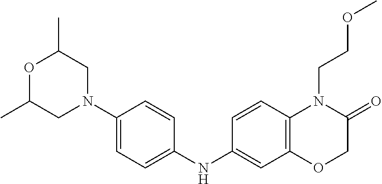

The title compound 95 (56 mg) was prepared in a yield of 25% as a grey solid from 7-bromo-4-(2-methoxyethyl)-2H-benzo[b][1,4]oxazin-3(4H)-one (150 mg, 0.59 mmol), 4-(2,6-dimethylmorpholino)aniline (110 mg, 0.53 mmol), 1,4-dioxane (2 mL), Pd₂(dba)₃ (49 mg, 0.05 mmol), Xphos (51 mg, 0.11 mmol) and Cs₂CO₃ (348 mg, 1.06 mmol) according to the procedure for compound 1. ¹H NMR (400 MHz, DMSO-d₆) δ 7.77 (s, 1H), 7.03 (d, J=8.8 Hz, 1H), 6.97-6.89 (m, 2H), 6.89-6.80 (m, 2H), 6.55 (dd, J=8.7, 2.5 Hz, 1H), 6.49 (d, J=2.5 Hz, 1H), 4.51 (s, 2H), 3.98 (t, J=5.8 Hz, 2H), 3.71-3.59 (m, 2H), 3.46 (t, J=5.8 Hz, 2H), 3.44-3.37 (m, 2H), 3.20 (s, 3H), 2.20-2.10 (m, 2H), 1.10 (d, J=6.2 Hz, 6H).

### Compound 96

### 7-((4-(2,6-dimethylmorpholino)phenyl)amino)-2H-benzo[b][1,4]oxazin-3(4H)-one

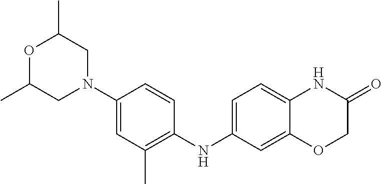 [SMILES: Cc1cc(N2CC(C)OC(C)C2)ccc1Nc1ccc2c(c1)OCC(=O)N2]

The title compound 96 (20 mg) was prepared in a yield of 8% as a grey solid from 7-bromo-2H-benzo[b][1,4]oxazin-3(4H)-one (154 mg, 0.68 mmol), 4-(2,6-dimethylmorpholino)-2-methylaniline (150 mg, 0.68 mmol), 1,4-dioxane (2 mL), Brettphos Pd G3 (62 mg, 0.14 mmol), Brettphos (73 mg, 0.13 mmol) and Cs₂CO₃ (444 mg, 1.36 mmol) according to the procedure for compound 1. ¹H NMR (400 MHz, DMSO-d₆) δ 10.38 (s, 1H), 7.08 (s, 1H), 6.96 (d, J=8.6 Hz, 1H), 6.82 (d, J=2.8 Hz, 1H), 6.73 (dd, J=8.6, 2.8 Hz, 1H), 6.65 (d, J=8.4 Hz, 1H), 6.26 (dd, J=8.4, 2.4 Hz, 1H), 6.23 (d, J=2.3 Hz, 1H), 4.45 (s, 2H), 3.74-3.62 (m, 2H), 3.53-3.45 (m, 2H), 2.29-2.14 (m, 2H), 2.11 (s, 3H), 1.14 (d, J=6.2 Hz, 6H). Mass (m/z): 368.4 [M+H]⁺.

### Compound 97

### 7-((4-((1S,4S)-2-oxa-5-azabicyclo[2.2.1]heptan-5-yl)-3-fluorophenyl) amino)-4-methyl-2H-benzo[b][1,4]oxazin-3(4H)-one

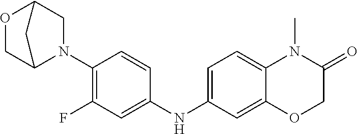 [SMILES: CN1C(=O)COc2cc(Nc3ccc(N4CC5CC4CO5)c(F)c3)ccc21]

The title compound 97 (47.5 mg) was prepared in a yield of 26% as a light yellow solid from 7-bromo-4-methyl-2H-benzo[b][1,4]oxazin-3(4H)-one (120 mg, 0.49 mmol), 4-((1S,4S)-2-oxa-5-azabicyclo[2.2.1] heptan-5-yl)-3-fluoroaniline (103.2 mg, 0.49 mmol), 1,4-dioxane (5 mL), Pd₂(dba)₃ (22.7 mg, 0.025 mmol), X-phos (23.6 mg, 0.049 mmol) and Cs₂CO₃ (323 mg, 0.99 mmol) according to the procedure for compound 1. ¹H NMR (400 MHz, DMSO-d₆) δ 7.91 (s, 1H), 6.99 (d, J=8.7 Hz, 1H), 6.85-6.76 (m, 1H), 6.76-6.68 (m, 2H), 6.65 (dd, J=8.7, 2.5 Hz, 1H), 6.56 (d, J=2.4 Hz, 1H), 4.58 (s, 2H), 4.53 (t, J=1.9 Hz, 1H), 4.37 (s, 1H), 3.81 (d, J=7.6 Hz, 1H), 3.72 (dd, J=7.7, 1.6 Hz, 1H), 3.56 (ddd, J=9.7, 4.0, 1.7 Hz, 1H), 3.23 (s, 3H), 2.99 (dd, J=9.8, 3.5 Hz, 1H), 1.88 (dd, J=9.6, 2.3 Hz, 1H), 1.83-1.75 (m, 1H). LC-MS (m/z) 370.2 [M+H]⁺.

### Compound 98

### 7-((4-(2-oxa-5-azabicyclo[2.2.2]octan-5-yl)-3-fluorophenyl)amino)-4-methyl-2H-benzo[b][1,4]oxazin-3(4H)-one

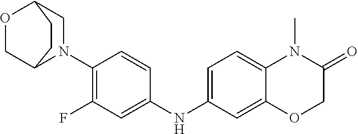 [SMILES: CN1C(=O)COc2cc(Nc3ccc(N4CC5CCC4CO5)c(F)c3)ccc21]

The title compound 98 (34.5 mg) was prepared in a yield of 16.7% as an off-white solid from 7-bromo-4-methyl-2H-benzo[b][1,4]oxazin-3(4H)-one (120 mg, 0.49 mmol), 4-(2-oxa-5-azabicyclo[2.2.2]octan-5-yl)-3-fluoroaniline (110 mg, 0.49 mmol), 1,4-dioxane (5 mL), Pd₂(dba)₃ (22.7 mg, 0.025 mmol), X-phos (23.6 mg, 0.049 mmol) and Cs₂CO₃ (323 mg, 0.99 mmol) according to the procedure for compound 1. ¹H NMR (400 MHz, DMSO-d₆) δ 7.94 (s, 1H), 7.00 (d, J=8.7 Hz, 1H), 6.89-6.84 (m, 1H), 6.84-6.81 (m, 1H), 6.80-6.73 (m, 1H), 6.66 (dd, J=8.7, 2.5 Hz, 1H), 6.57 (d, J=2.5 Hz, 1H), 4.58 (s, 2H), 4.09 (dt, J=8.9, 2.4 Hz, 1H), 3.92-3.84 (m, 2H), 3.69 (dq, J=10.5, 2.8 Hz, 1H), 3.54 (d, J=3.0 Hz, 1H), 3.32 (d, J=2.5 Hz, 1H), 3.23 (s, 3H), 2.08 (s, 1H), 2.02-1.93 (m, 1H), 1.88-1.67 (m, 2H). LC-MS (m/z) 384.2 [M+H]⁺.

### Compound 99

### 7-((4-(6-oxa-3-azabicyclo[3.1.1]heptan-3-yl)-3-fluorophenyl)amino)-4-methyl-2H-benzo[b][1,4]oxazin-3(4H)-one

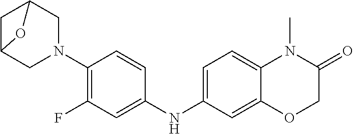 [SMILES: CN1C(=O)COc2cc(Nc3ccc(N4CC5CC(C4)O5)c(F)c3)ccc21]

The titled compound 99 (21.7 mg, 14.2%) as an off-white solid was prepared according to the procedure outlined for compound 1. ¹H NMR (400 MHz, DMSO-d₆) δ 8.00 (s, 1H), 7.01 (d, J=8.7 Hz, 1H), 6.97 (dd, J=10.4, 8.6 Hz, 1H), 6.86-6.77 (m, 2H), 6.69 (dd, J=8.7, 2.5 Hz, 1H), 6.60 (d, J=2.4 Hz, 1H), 4.58 (d, J=3.7 Hz, 4H), 3.55 (d, J=11.3 Hz, 2H), 3.23 (s, 3H), 3.43 (d, J=11.4 Hz, 2H), 3.04 (dt, J=8.1, 6.3 Hz, 1H), 2.17 (d, J=8.2 Hz, 1H). LC-MS (m/z) 370.2 [M+H]⁺.

### Compound 100

### 7-((2-methyl-4-(4-(trifluoromethyl)piperidin-1-yl)phenyl)amino)-2H-benzo[b][1,4]oxazin-3(4H)-one

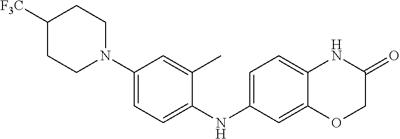 [SMILES: Cc1cc(N2CCC(C(F)(F)F)CC2)ccc1Nc1ccc2c(c1)OCC(=O)N2]

The title compound 100 (32 mg) was prepared in a yield of 9.0% as a white solid from 7-bromo-2H-benzo[b][1,4]oxazin-3(4H)-one (200 mg, 0.88 mmol), 2-methyl-4-(4-(trifluoromethyl)piperidin-1-yl)aniline (226 mg, 0.88 mmol), t-BuOH (5 mL), Pd₂(dba)₃ (40 mg, 0.045 mmol), Brettphos (47.1 mg, 0.088 mmol) and Cs₂CO₃ (572 mg, 1.75 mmol) according to the procedure for compound 1. ¹H NMR (400 MHz, DMSO-d₆) δ 10.39 (s, 1H), 7.08 (s, 1H), 6.96 (d, J=8.6 Hz, 1H), 6.84 (d, J=2.8 Hz, 1H), 6.75 (dd, J=8.7, 2.8 Hz, 1H), 6.65 (d, J=8.4 Hz, 1H), 6.28 (dd, J=8.4, 2.4 Hz, 1H), 6.24 (d, J=2.3 Hz, 1H), 4.45 (s, 2H), 3.68 (d, J=12.3 Hz, 2H), 2.62 (dd, J=12.4, 2.5 Hz, 2H), 2.47-2.38 (m, 1H), 2.11 (s, 3H), 1.92-1.83 (m, 2H), 1.56 (qd, J=12.5, 4.1 Hz, 2H). LC-MS (m/z) 406.2 [M+H]⁺.

### Compound 101

### 7-((3-methyl-4-(4-(trifluoromethyl)piperidin-1-yl)phenyl)amino)-2H-benzo[b][1,4]oxazin-3(4H)-one

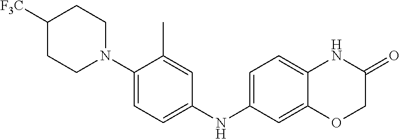 [SMILES: Cc1cc(Nc2ccc3c(c2)OCC(=O)N3)ccc1N1CCC(C(F)(F)F)CC1]

The titled compound 101 (113 mg, 31.8%) as a white solid was prepared according to the procedure outlined for compound 1. LC-MS (m/z) 406.2 [M+H]⁺. ¹H NMR (400 MHz, DMSO-d₆) δ 10.48 (s, 1H), 7.79 (s, 1H), 6.91 (d, J=8.3 Hz, 1H), 6.82 (d, J=8.8 Hz, 2H), 6.73 (d, J=8.4 Hz, 1H), 6.60 (dd, J=8.4, 2.3 Hz, 1H), 6.56 (d, J=2.3 Hz, 1H), 4.49 (s, 2H), 3.02 (d, J=11.5 Hz, 2H), 2.60 (td, J=11.9, 2.3 Hz, 2H), 2.43-2.32 (m, 1H), 2.18 (s, 3H), 1.92-1.84 (m, 2H), 1.60 (qd, J=12.3, 4.0 Hz, 2H).

### Compound 102

### 1,4-dimethyl-6-((4-(4-methylpiperidin-1-yl)phenyl)amino)-3,4-dihydroquinoxalin-2(1H)-one

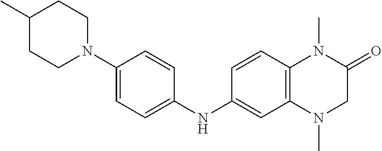 [SMILES: CC1CCN(c2ccc(Nc3ccc4c(c3)N(C)CC(=O)N4C)cc2)CC1]

The title compound 102 (8.5 mg) was prepared in a total yield of 11.9% as a purple solid from 4-(4-methylpiperidin-1-yl)aniline (48 mg, 0.256 mmol) and 6-bromo-1,4-dimethyl-3,4-dihydroquinoxalin-2(1H)-one (50 mg, 0.197 mmol) in 1,4-dioxane (5 mL), Pd₂(dba)₃ (2 mg, 0.002 mmol), X-phos (6 mg, 0.01 mmol), Cs₂CO₃ (97 mg, 0.295 mmol) and 1,4-dioxane (5 mL) according to the procedure for compound 1. ¹H NMR (400 MHz, Methanol-d₄) δ 7.07 (d, J=8.4 Hz, 2H), 7.00 (d, J=8.4 Hz, 3H), 6.62 (d, J=8.4 Hz, 1H), 6.54 (s, 1H), 3.63 (d, J=7.2 Hz, 2H), 3.42 (s, 3H), 3.37 (s, 2H), 3.10 (s, 3H), 2.69 (s, 2H), 2.29 (dq, J=8.4, 4.8, 4.4 Hz, 1H), 2.01 (s, 1H), 1.98 (s, 1H), 1.76 (d, J=4.0 Hz, 1H), 1.73 (d, J=4.0 Hz, 1H), 1.73 (d, J=4.0 Hz, 1H). Mass (m/z): 365.3 [M+H]⁺.

### Compound 103

### 2-(4-methylpiperazin-1-yl)-1-(6-((4-(piperidin-1-yl)phenyl)amino)-3,4-dihydroisoquinolin-2(1H)-yl)ethan-1-one

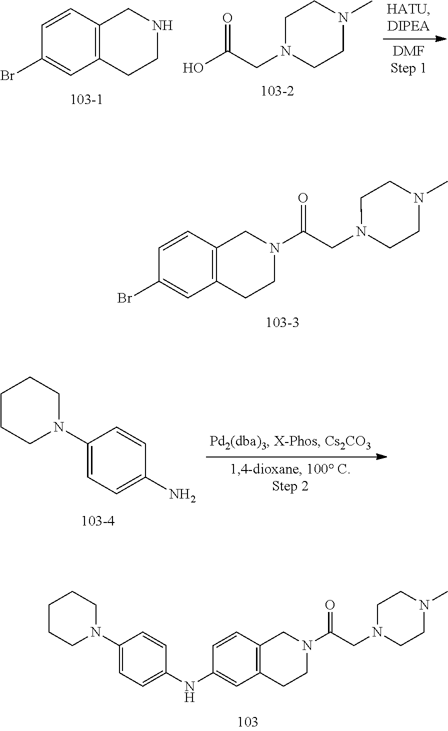

### Step 1. Preparation of 1-(6-bromo-3,4-dihydroisoquinolin-2(1H)-yl)-2-(4-methylpiperazin-1-yl)ethan-1-one (103-3)

To a solution of 6-bromo-1,2,3,4-tetrahydroisoquinoline (212 mg, 1.0 mmol) and 2-(4-methylpiperazin-1-yl)acetic acid (205 mg, 1.3 mmol) in DMF (5 mL) was added DIEA (0.33 mL, 2.0 mmol). Followed by the addition of HATU (501 mg, 1.3 mmol) then the reaction mixture was stirred for 3 hours at r.t. Then 40 ml of water was added. Then the mixture was extracted by DCM (20 mL×3). The combined organic layers were washed with water (20 mL×3), dried over Na₂SO₄ and concentrated under vacuum. The residue was purified by prep-TLC to give the desired product as yellow solid (301 mg, 85.2%). Mass (m/z): 352.1 [M+H]⁺.

### Step 2. Preparation of 2-(4-methylpiperazin-1-yl)-1-(6-((4-(piperidin-1-yl) phenyl)amino)-3,4-dihydroisoquinolin-2(1H)-yl)ethan-1-one (103)

The title compound 103 (18.5 mg) was prepared in a total yield of 82.6% as a yellow solid from 4-(piperidin-1-yl)aniline (11.4 mg, 0.065 mmol), 1-(6-bromo-3,4-dihydroisoquinolin-2(1H)-yl)-2-(4-methylpiperazin-1-yl)e than-1-one (17.6 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.40-6.73 (m, 7H), 4.72 (s, 2H), 3.83 (s, 2H), 3.51-3.39 (m, 4H), 3.06-2.87 (m, 4H), 2.85-2.63 (m, 8H), 2.50 (s, 3H), 1.90-1.76 (m, 4H), 1.71-1.60 (m, 2H). Mass (m/z): 448.3 [M+H]⁺.

### Compound 104

### N-phenyl-1,2,3,4-tetrahydroisoquinolin-6-amine

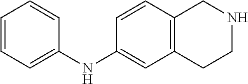 [SMILES: c1ccc(Nc2ccc3c(c2)CCNC3)cc1]

The title compound 104 (8.6 mg) was prepared in a total yield of 76.4% as an orange solid from 6-bromo-1,2,3,4-tetrahydroisoquinoline (10.6 mg, 0.05 mmol), aniline (6.1 mg, 0.065 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.29-7.18 (m, 2H), 7.09-6.79 (m, 6H), 4.10 (s, 2H), 3.29 (t, J=6.4 Hz, 2H), 2.93 (t, J=6.4 Hz, 2H). Mass (m/z): 225.3 [M+H]⁺.

### Compound 105

### 2-ethyl-N-phenyl-1,2,3,4-tetrahydroisoquinolin-6-amine

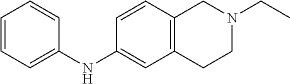 [SMILES: CCN1CCc2cc(Nc3ccccc3)ccc2C1]

The title compound 105 (9.8 mg) was prepared in a total yield of 77.8% as a orange solid from 6-bromo-2-ethyl-1,2,3,4-tetrahydroisoquinoline (12.0 mg, 0.05 mmol), aniline (6.1 mg, 0.065 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.24-7.17 (m, 2H), 7.08-7.02 (m, 2H), 7.00-6.80 (m, 4H), 3.89 (s, 2H), 3.08 (t, J=6.4 Hz, 2H), 2.97 (t, J=6.4 Hz, 2H), 2.91 (q, J=7.2 Hz, 2H), 1.29 (t, J=7.2 Hz, 3H). Mass (m/z): 253.2 [M+H]⁺.

### Compound 106

### 1-(6-(phenylamino)-3,4-dihydroisoquinolin-2(1H)-yl)ethan-1-one

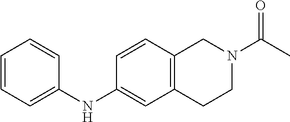 [SMILES: CC(=O)N1CCc2cc(Nc3ccccc3)ccc2C1]

The title compound 106 (9.2 mg) was prepared in a total yield of 68.9% as a orange solid from 1-(6-bromo-3,4-dihydroisoquinolin-2(1H)-yl) ethan-1-one (12.7 mg, 0.05 mmol), aniline (6.1 mg, 0.065 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.23-7.16 (m, 2H), 7.07-6.78 (m, 6H), 4.57 (s, 2H), 3.66 (t, J=6.4 Hz, 2H), 2.79 (t, J=6.4 Hz, 2H), 2.15 (s, 3H). Mass (m/z): 267.2 [M+H]⁺.

### Compound 107

### 2-(4-methylpiperazin-1-yl)-1-(6-(phenylamino)-3,4-dihydroisoquinolin-2(1H)-yl)ethan-1-one

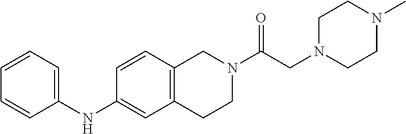 [SMILES: CN1CCN(CC(=O)N2CCc3cc(Nc4ccccc4)ccc3C2)CC1]

The title compound 107 (15.3 mg) was prepared in a total yield of 83.8% as an orange solid from 1-(6-bromo-3,4-dihydroisoquinolin-2(1H)-yl)-2-(4-methylpiperazin-1-yl)ethan-1-one (17.6 mg, 0.05 mmol), aniline (6.1 mg, 0.065 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.25-7.14 (m, 2H), 7.08-6.78 (m, 6H), 4.62 (s, 2H), 3.72 (s, 2H), 3.36 (t, J=6.0 Hz, 2H), 2.84 (t, J=6.0 Hz, 2H), 2.80-2.58 (m, 8H), 2.44 (s, 3H). Mass (m/z): 365.2 [M+H]⁺

### Compound 108

### 1-(6-((3-fluoro-4-(4-(trifluoromethyl)piperidin-1-yl)phenyl)amino)-3,4-dihydroisoquinolin-2(1H)-yl)-2-(4-methylpiperazin-1-yl)ethan-1-one

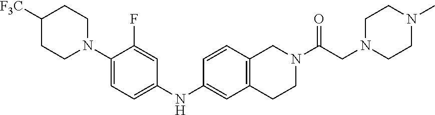 [SMILES: CN1CCN(CC(=O)N2CCc3cc(Nc4ccc(N5CCC(C(F)(F)F)CC5)c(F)c4)ccc3C2)CC1]

The title compound 108 (20.1 mg) was prepared in a total yield of 75.3% as a yellow solid from 3-fluoro-4-(4-(trifluoromethyl)piperidin-1-yl) aniline (17.1 mg, 0.065 mmol), 1-(6-bromo-3,4-dihydroisoquinolin-2(1H)-yl)-2-(4-methylpiperazin-1-yl)ethan-1-one (17.6 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.14-6.72 (m, 6H), 4.63 (s, 2H), 3.76 (s, 2H), 3.41-3.33 (m, 4H), 2.96-2.75 (m, 4H), 2.73-2.55 (m, 8H), 2.41 (s, 3H), 2.25 (m, 1H), 1.98-1.90 (m, 2H), 1.79-1.68 (m, 2H). Mass (m/z): 534.3 [M+H]⁺

### Compound 109

### 1,4-dimethyl-6-((4-(4-(trifluoromethyl)piperidin-1-yl)phenyl)amino)-3,4-dihydroquinoxalin-2(H)-one

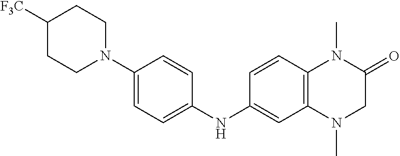 [SMILES: CN1CC(=O)N(C)c2ccc(Nc3ccc(N4CCC(C(F)(F)F)CC4)cc3)cc21]

The title compound 109 (29.5 mg) was prepared in a total yield of 35.8% as a purple solid from 4-(4-(trifluoromethyl)piperidin-1-yl)aniline (62 mg, 0.256 mmol), 6-bromo-1,4-dimethyl-3,4-dihydroquinoxalin-2(1H)-one (50 mg, 0.197 mmol), Pd₂(dba)₃ (2 mg, 0.002 mmol), X-phos (6 mg, 0.01 mmol), Cs₂CO₃ (97 mg, 0.295 mmol) and 1,4-dioxane (5 mL) according to the procedure for compound 1. ¹H NMR (400 MHz, Methanol-d₄) δ 7.07 (d, J=8.4 Hz, 2H), 7.00 (d, J=8.4 Hz, 3H), 6.62 (d, J=8.4 Hz, 1H), 6.54 (s, 1H), 3.63 (d, J=7.2 Hz, 2H), 3.42 (s, 3H), 3.37 (s, 2H), 3.10 (s, 3H), 2.69 (s, 2H), 2.29 (dq, J=8.4, 4.8, 4.4 Hz, 1H), 2.01 (s, 1H), 1.98 (s, 1H), 1.76 (d, J=4.0 Hz, 1H), 1.73 (d, J=4.0 Hz, 1H). Mass (m/z): 419.3 [M+H]⁺.

### Compound 110

### 1,4-dimethyl-6-((4-(4-(trifluoromethyl)piperidin-1-yl)phenyl)amino)-1,4-dihydroquinoxaline-2,3-dione

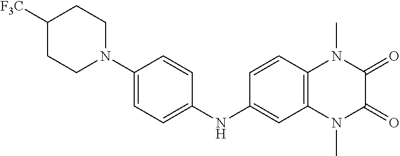 [SMILES: Cn1c(=O)c(=O)n(C)c2cc(Nc3ccc(N4CCC(C(F)(F)F)CC4)cc3)ccc21]

The title compound 110 (8.8 mg) as prepared in a total yield of 43.4% as a yellow powder from 4-(4-(trifluoromethyl)piperidin-1-yl)aniline (80 mg, 0.33 mmol), 6-bromo-1,4-dimethyl-1,4-dihydroquinoxaline-2,3-dione (67 mg, 0.25 mmol), Pd₂(dba)₃ (2.3 mg, 2.5 umol), X-Phos (6.0 mg, 12.5 umol), t-BuOK (36 mg, 0.38 mmol) according to the procedure for compound 1. ¹H NMR (400 MHz, Methanol-d₄) δ 7.30 (d, J=8.8 Hz, 1H), 7.16-7.08 (m, 2H), 7.04-6.94 (m, 4H), 3.72-3.66 (m, 2H), 3.65 (s, 3H), 3.60 (s, 3H), 2.76-2.65 (m, 2H), 2.35-2.25 (m, 1H), 2.03-1.96 (m, 1H), 1.75 (qd, J=12.5, 4.1 Hz, 2H). Mass (m/z): 433.3 [M+H]⁺.

### Compound 111

### 1-methyl-6-((4-(4-(trifluoromethyl)piperidin-1-yl)phenyl)amino) quinoxalin-2(1H)-one

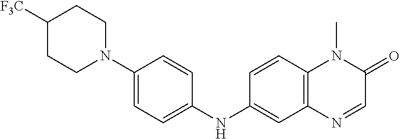 [SMILES: Cn1c(=O)cnc2cc(Nc3ccc(N4CCC(C(F)(F)F)CC4)cc3)ccc21]

The title compound 111 (37.9 mg) was prepared in a total yield of 22.6% as a yellow solid from 4-(4-(trifluoromethyl)piperidin-1-yl)aniline (122 mg, 0.502 mmol), 6-bromo-1-methylquinoxalin-2(1H)-one (100 mg, 0.418 mmol), Pd₂(dba)₃ (4 mg, 0.004 mmol), X-phos (10 mg, 0.02 mmol) and Cs₂CO₃ (206 mg, 0.628 mmol) and 1,4-dioxane (5 mL) according to the procedure for compound 1. ¹H NMR (400 MHz, DMSO-d₆) δ 8.16 (s, 1H), 8.06 (s, 1H), 7.48-7.42 (m, 1H), 7.28 (d, J=7.6 Hz, 2H), 7.06-7.03 (m, 2H), 6.97-6.92 (m, 2H), 3.66 (d, J=12.0 Hz, 2H), 3.57 (s, 3H), 2.65 (td, J=12.0, 2.4 Hz, 2H), 2.43 (ddd, J=12.4, 8.4, 3.6 Hz, 1H), 1.93-1.85 (m, 2H), 1.58 (qd, J=12.4, 4.0 Hz, 2H). Mass (m/z): 403.3 [M+H]⁺.

### Compound 112

### 1,3-dimethyl-6-((4-(4-(trifluoromethyl)piperidin-1-yl)phenyl)amino)-3,4-dihydroquinazolin-2(1H)-one

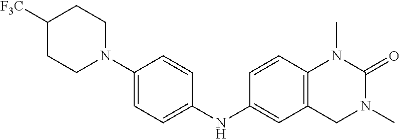 [SMILES: CN1Cc2cc(Nc3ccc(N4CCC(C(F)(F)F)CC4)cc3)ccc2N(C)C1=O]

The title compound 112 (11.3 mg) was prepared in a total yield of 6.9% as a purple solid from 4-(4-(trifluoromethyl)piperidin-1-yl)aniline (115 mg, 0.470 mmol), 6-bromo-1,3-dimethyl-3,4-dihydroquinazolin-2(1H)-one (100 mg, 0.392 mmol), Pd₂(dba)₃ (4 mg, 0.004 mmol), X-phos (10 mg, 0.02 mmol) and Cs₂CO₃ (193 mg, 0.588 mmol) and 1,4-dioxane (5 mL) according to the procedure for compound 1. ¹H NMR (400 MHz, Methanol-d₄) δ 7.07 (d, J=8.4 Hz, 2H), 7.00 (d, J=8.4 Hz, 3H), 6.62 (d, J=8.4 Hz, 1H), 6.54 (s, 1H), 3.63 (d, J=7.2 Hz, 2H), 3.42 (s, 3H), 3.37 (s, 2H), 3.10 (s, 3H), 2.69 (s, 2H), 2.29 (dq, J=8.4, 4.8, 4.4 Hz, 1H), 2.01 (s, 1H), 1.98 (s, 1H), 1.76 (d, J=4.0 Hz, 1H), 1.73 (d, J=4.0 Hz, 1H). Mass (m/z): 419.3 [M+H]⁺.

### Compound 113

### 1,4-dimethyl-6-((4-(4-(trifluoromethyl)piperidin-1-yl)phenyl)amino) quinazolin-2(1H)-one

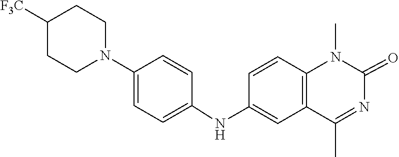 [SMILES: Cc1nc(=O)n(C)c2ccc(Nc3ccc(N4CCC(C(F)(F)F)CC4)cc3)cc12]

The title compound 113 (33.4 mg) was prepared in a total yield of 20.3% as a pink solid from 4-(4-(trifluoromethyl)piperidin-1-yl)aniline (116 mg, 0.474 mmol), 6-bromo-1,4-dimethylquinazolin-2(1H)-one (100 mg, 0.395 mmol), Pd₂(dba)₃ (4 mg, 0.004 mmol), X-phos (10 mg, 0.02 mmol), Cs₂CO₃ (194 mg, 0.592 mmol) and 1,4-dioxane (5 mL) according to the procedure for compound 1. ¹H NMR (400 MHz, DMSO-d₆) δ 10.01 (s, 1H), 8.72 (s, 1H), 7.85 (s, 2H), 7.50 (s, 1H), 7.11 (s, 5H), 3.88-3.65 (m, 6H), 2.92 (d, J=44.4 Hz, 4H), 1.94 (s, 3H), 1.65 (s, 2H). Mass (m/z): 417.3 [M+H]⁺.

### Compound 114

### 4-methyl-N-(4-(4-(trifluoromethyl)piperidin-1-yl)phenyl)-3,4-dihydro-2H-benzo[b][1,4]oxazin-7-amine

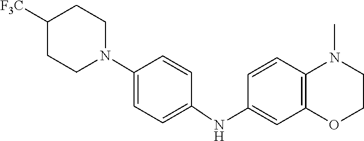 [SMILES: CN1CCOc2cc(Nc3ccc(N4CCC(C(F)(F)F)CC4)cc3)ccc21]

The title compound 114 (46.7 mg) was prepared in a total yield of 54.6% as a green solid from 4-(4-(trifluoromethyl)piperidin-1-yl)aniline (64 mg, 0.263 mmol), 7-bromo-4-methyl-3,4-dihydro-2H-benzo[b][1,4]oxazine (50 mg, 0.219 mmol), Pd₂(dba)₃ (2 mg, 0.002 mmol), X-phos (6 mg, 0.01 mmol), Cs₂CO₃ (108 mg, 0.328 mmol) and 1,4-dioxane (5 mL) according to the procedure for compound 1. ¹H NMR (400 MHz, DMSO-d₆) δ 7.82 (s, 1H), 6.97 (dt, J=10.0, 3.2 Hz, 3H), 6.94-6.87 (m, 2H), 6.63 (dd, J=8.8, 2.4 Hz, 1H), 6.55 (d, J=2.4 Hz, 1H), 4.57 (s, 2H), 3.77 (d, J=12.0, 2H), 0.63 (d, J=11.6 Hz, 2H), 3.23 (s, 3H), 2.63 (td, J=12.4, 2.4 Hz, 2H), 2.43 (dp, J=12.4, 4.0 Hz, 1H), 1.88 (d, J=12.0 Hz, 2H), 1.57 (qd, J=12.4, 4.0 Hz, 2H). Mass (m/z): 392.3 [M+H]⁺.

### Compound 115

### 1-methyl-6-((4-(4-methylpiperidin-1-yl)phenyl)amino)quinoxalin-2(1H)-one

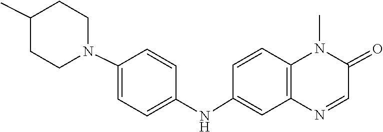 [SMILES: CC1CCN(c2ccc(Nc3ccc4c(c3)ncc(=O)n4C)cc2)CC1]

The title compound 115 (101.2 mg) was prepared in a total yield of 69.5% as a yellow solid from 4-(4-methylpiperidin-1-yl)aniline (95 mg, 0.502 mmol), 6-bromo-1-methylquinoxalin-2(1H)-one (100 mg, 0.418 mmol), Pd₂(dba)₃ (4 mg, 0.004 mmol), X-phos (12 mg, 0.02 mmol), Cs₂CO₃ (206 mg, 0.627 mmol) and 1,4-dioxane (5 mL) according to the procedure for compound 1. ¹H NMR (400 MHz, DMSO-d₆) δ 8.74 (s, 1H), 8.22 (s, 1H), 7.63-7.40 (m, 5H), 7.19 (d, J=8.5 Hz, 2H), 4.71 (s, 4H), 3.60 (s, 3H), 1.90 (d, J=13.8 Hz, 2H), 1.77 (d, J=7.8 Hz, 1H), 1.59 (d, J=12.6 Hz, 2H), 0.99 (d, J=6.3 Hz, 3H). Mass (m/z): 349.3 [M+H]⁺.

### Compound 116

### 1-ethyl-6-((4-(4-(trifluoromethyl)piperidin-1-yl)phenyl)amino) quinoxalin-2(1H)-one

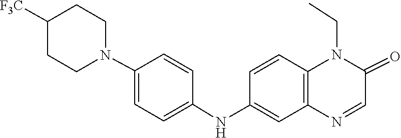 [SMILES: CCn1c(=O)cnc2cc(Nc3ccc(N4CCC(C(F)(F)F)CC4)cc3)ccc21]

The title compound 116 (31.6 mg) was prepared in a total yield of 38.10% as a yellow solid from 4-(4-(trifluoromethyl)piperidin-1-yl)aniline (58 mg, 0.239 mmol), 6-bromo-1-ethylquinoxalin-2(1H)-one (50 mg, 0.199 mmol), Pd₂(dba)₃ (2 mg, 0.002 mmol), X-phos (6 mg, 0.01 mmol), Cs₂CO₃ (98 mg, 0.299 mmol) and 1,4-dioxane (5 mL) according to the procedure for compound 1. ¹H NMR (400 MHz, DMSO-d₆) δ 8.17 (s, 1H), 7.69-6.81 (m, 7H), 4.22 (q, J=7.2 Hz, 4H), 3.07 (s, 2H), 2.62 (d, J=10.0 Hz, 1H), 2.10-1.92 (m, 2H), 1.80 (d, J=36.0 Hz, 2H), 1.23 (t, J=7.2 Hz, 3H). Mass (m/z): 417.3 [M+H]⁺.

### Compound 117

### 1-ethyl-6-((4-(4-methylpiperidin-1-yl)phenylamino) quinoxalin-2(1H)-one

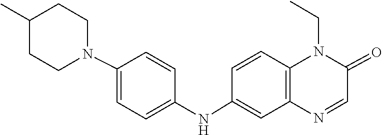 [SMILES: CCn1c(=O)cnc2cc(Nc3ccc(N4CCC(C)CC4)cc3)ccc21]

The title compound 117 (40.2 mg) was prepared in a total yield of 55.8% as a yellow solid from 4-(4-methylpiperidin-1-yl)aniline (45 mg, 0.239 mmol), 6-bromo-1-ethylquinoxalin-2(1H)-one (50 mg, 0.199 mmol), Pd₂(dba)₃ (2 mg, 0.002 mmol), X-phos (6 mg, 0.01 mmol), Cs₂CO₃ (98 mg, 0.299 mmol) and 1,4-dioxane (5 mL) according to the procedure for compound 1. ¹H NMR (400 MHz, DMSO-d₆) δ 8.81-8.54 (m, 1H), 8.20 (s, 1H), 7.72-7.03 (m, 7H), 4.23 (q, J=7.2 Hz, 2H), 3.71-3.60 (m, 4H), 2.00-1.46 (m, 5H), 1.24 (t, J=7.2 Hz, 3H), 0.98 (d, J=6.0 Hz, 3H). Mass (m/z): 363.3 [M+H]⁺.

### Compound 118

### 7-((4-(tert-butyl)phenyl)amino)-4-methyl-2H-chromen-2-one

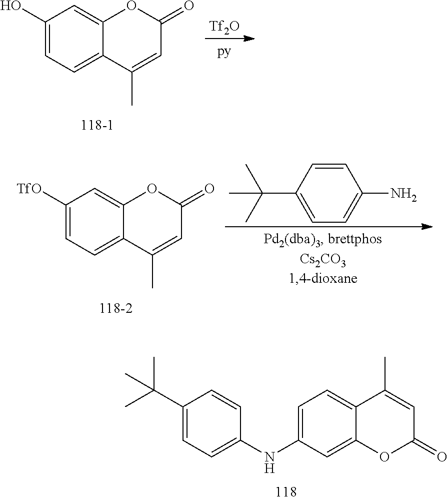

### Step 1. Preparation of 4-methyl-2-oxo-2H-chromen-7-yl trifluoromethanesulfonate (118-2)

To a solution of 7-hydroxy-4-methyl-2H-chromen-2-one (500 mg, 2.84 mmol) in pyridine (10 mL) was added trifluoromethanesulfonic anhydride (881 mg, 3.13 mmol) at 0° C. Then the mixture was stirred at room temperature for 1 hour. The mixture was diluted with water and extracted by EA (25 mL×3). The combined organic layers were washed with brine (15 mL×3), dried over Na₂SO₄ and concentrated to give the crude product which was purified by Column chromatography to give the desired product 600 mg as a yellow solid. Mass (m/z): 309.3 [M+H]⁺

### Step 2. Preparation of 7-((4-(tert-butyl)phenyl)amino)-4-methyl-2H-chromen-2-one (118)

To a solution of 4-methyl-2-oxo-2H-chromen-7-yl trifluoromethanesulfonate (100 mg, 0.324 mmol) and 4-(tert-butyl)aniline (53.2 mg, 0.356 mmol) in dioxane (5 mL) was added Pd₂(dba)₃ (14.8 mg, 0.016 mmol), Brettphos (17.4 mg, 0.0324 mmol) and Cs₂CO₃ (212 mg, 0.648 mmol) under nitrogen atmosphere. Then the mixture was stirred at 100° C. overnight. The mixture was extracted by EA (25 mL×3). The combined organic layers were washed with brine (15 mL×3), dried over Na₂SO₄ and concentrated to give the crude product, which was purified by pre-HPLC to give the desired product as yellow solid (48.8 mg, 49.0%). ¹H NMR (400 MHz, DMSO-d₆) δ 8.78 (s, 1H), 7.53 (d, J=8.8 Hz, 1H), 7.37-7.29 (m, 2H), 7.15-7.07 (m, 2H), 6.92 (dd, J=8.8, 2.3 Hz, 1H), 6.78 (d, J=2.3 Hz, 1H), 6.01 (d, J=1.3 Hz, 1H), 2.31 (d, J=1.2 Hz, 3H), 1.25 (s, 9H). Mass (m/z): 308.4 [M+H]⁺.

### Compound 119

### 4-methyl-7-((4-(4-(trifluoromethyl)piperidin-1-yl)phenyl)amino)-2H-chromen-2-one

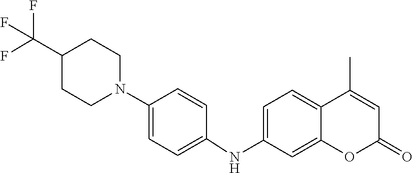 [SMILES: Cc1cc(=O)oc2cc(Nc3ccc(N4CCC(C(F)(F)F)CC4)cc3)ccc12]

The title compound 119 was prepared according to the procedure for compound 118. To a solution of 4-methyl-2-oxo-2H-chromen-7-yl trifluoromethanesulfonate (100 mg, 0.325 mmol) and 4-(4-(trifluoromethyl)piperidin-1-yl)aniline (79.2 mg, 0.325 mmol) in dioxane (5 mL) was added Pd₂(dba)₃ (14.9 mg, 0.0163 mmol), Brettphos (17.5 mg, 0.0325 mmol) and Cs₂CO₃ (212 mg, 0.65 mmol) under nitrogen atmosphere. Then the mixture was stirred at 100° C. overnight. The mixture was extracted by EA (25 mL×3). The combined organic layers were washed with brine (15 mL×3), dried over Na₂SO₄ and concentrated to give the crude product, which was purified by pre-HPLC to give the desired product as yellow solid (34.1 mg, 26.1%). ¹H NMR (400 MHz, DMSO-d₆) δ 8.59 (s, 1H), 7.49 (d, J=8.8 Hz, 1H), 7.10-7.02 (m, 2H), 6.99-6.90 (m, 2H), 6.80 (dd, J=8.8, 2.3 Hz, 1H), 6.64 (d, J=2.2 Hz, 1H), 5.96 (d, J=1.3 Hz, 1H), 3.68 (d, J=12.3 Hz, 2H), 2.65 (td, J=12.4, 2.5 Hz, 2H), 2.30 (d, J=1.2 Hz, 3H), 1.90-1.81 (m, 2H), 1.54 (qd, J=12.5, 4.1 Hz, 2H). Mass (m/z): 403.4 [M+H]⁺.

### Compound 120

### 6-((4-(tert-butyl)phenyl)amino)-2H-chromen-2-one

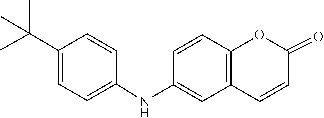 [SMILES: CC(C)(C)c1ccc(Nc2ccc3oc(=O)ccc3c2)cc1]

The title compound 120 (40.2 mg) was prepared according to the procedure for compound 118. To a solution of 2-oxo-2H-chromen-6-yl trifluoromethanesulfonate (100 mg, 0.34 mmol) and 4-(tert-butyl)aniline (55.8 mg, 0.374 mmol) in dioxane (5 mL) was added Pd₂(dba)₃ (15.6 mg, 0.017 mmol), Brettphos (18.3 mg, 0.034 mmol) and Cs₂CO₃ (222 mg, 0.68 mmol) under nitrogen atmosphere. Then the mixture was stirred at 100° C. overnight. The mixture was extracted by EA (25 mL×3). The combined organic layers were washed with brine (15 mL×3), dried over Na₂SO₄ and concentrated to give the crude product, which was purified by pre-HPLC to give the desired product as yellow solid (80 mg, 80%). ¹H NMR (400 MHz, CDCl3) δ 7.60 (d, J=9.5 Hz, 1H), 7.33 (d, J=8.5 Hz, 2H), 7.21 (dd, J=11.1, 8.9 Hz, 2H), 7.13 (s, 1H), 7.02 (t, J=9.7 Hz, 2H), 6.40 (d, J=9.5 Hz, 1H), 1.32 (s, 9H). Mass (m/z): 294.3 [M+H]⁺.

### Compound 121

### 4-(2-aminoethyl)-7-((4-(tert-butyl)phenyl)amino)-2H-chromen-2-one

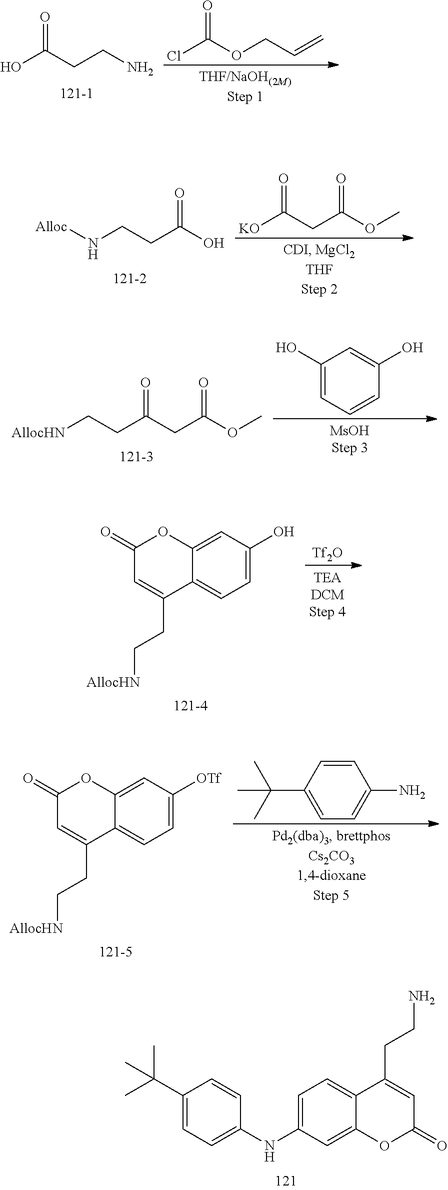

Step 1. Preparation of 3-(((allyloxy)carbonyl)amino)propanoic acid (121-2)

To a solution of 3-aminopropanoic acid (3.0 g, 33.7 mmol) in THF (60 mL)/H₂O (30 mL) was added NaOH (2N) (33.7 mL, 67.4 mmol), allyl carbonochloridate (5.9 mL, 33.7 mmol). Then the mixture was stirred at room temperature overnight. The mixture was diluted with water and extracted by DCM (25 mL×3). The combined organic layers were washed with brine (15 mL×3), dried over Na₂SO₄ and concentrated to give the crude product which was purified by Column chromatography to give the desired product 3.2 g as colorless oil. Mass (m/z): 174.2 [M+H]⁺.

### Step 2. Preparation of methyl 5-(((allyloxy)carbonyl)amino)-3-oxopentanoate (121-3)

To a solution of 3-(((allyloxy)carbonyl)amino)propanoic acid (2.0 g, 11.6 mmol) in THF (50 mL) was added CDI (2.26 g, 13.9 mmol) under argon atmosphere, the mixture was stirred at room temperature for 2 hours. Then a mixture of MgCl₂ (1.1 g, 11.6 mmol) and potassium 3-methoxy-3-oxopropanoate (2.71 g, 17.3 mmol) was added. The mixture was stirred at room temperature overnight. Then it was diluted with water and extracted by EA (25 mL×3). The combined organic layers were washed with brine (15 mL×3), dried over Na₂SO₄ and concentrated to give the crude product which was purified by Column chromatography to give the desired product 1.86 g as colorless oil. Mass (m/z): 230.2 [M+H]⁺.

### Step 3. Preparation of allyl (2-(7-hydroxy-2-oxo-2H-chromen-4-yl)ethyl) carbamate (121-4)

To a solution of methyl 5-(((allyloxy)carbonyl)amino)-3-oxopentanoate (1.86 g, 8.12 mmol) in MsOH (20 mL) was added resorcinol (893 g, 8.12 mmol) under argon atmosphere at 0° C., the mixture was stirred at 0° C. for 3 hours. Then saturated NaHSO₄ solution was added. The mixture was stirred from turbidity to clarification. Then it was diluted with water and extracted by EA (25 mL×3). The combined organic layers were washed with brine (15 mL×3), dried over Na₂SO₄ and concentrated to give the crude product which was purified by Column chromatography to give the desired product 1.0 g as yellow solid. Mass (m/z): 290.3 [M+H]⁺.

### Step 4. Preparation of 4-(2-(((allyloxy)carbonyl)amino)ethyl)-2-oxo-2H-chromen-7-yl trifluoromethanesulfonate (121-5)

To a solution of allyl (2-(7-hydroxy-2-oxo-2H-chromen-4-yl)ethyl) carbamate (1.0 g, 3.46 mmol) in DCM (20 mL) was added trifluoromethanesulfonic anhydride (1.07 g, 3.8 mmol), TEA (700 mg, 6.92 mmol) at 0° C. Then the mixture was stirred at room temperature for 3 hours. The mixture was diluted with water and extracted by DCM (25 mL×3). The combined organic layers were washed with brine (15 mL×3), dried over Na₂SO₄ and concentrated to give the crude product which was purified by Column chromatography to give the desired product 600 mg as yellow solid. Mass (m/z): 422.4 [M+H]⁺.

### Step 5. Preparation of 4-(2-aminoethyl)-7-((4-(tert-butyl)phenyl)amino)-2H-chromen-2-one (121)

To a solution of 4-(2-(((allyloxy)carbonyl)amino)ethyl)-2-oxo-2H-chromen-7-yl trifluoromethanesulfonate (100 mg, 0.238 mmol) and 4-(tert-butyl)aniline (35.4 mg, 0.238 mmol) in dioxane (5 mL) was added Pd₂(dba)₃ (10.9 mg, 0.012 mmol), Brettphos (12.8 mg, 0.0238 mmol) and Cs₂CO₃ (117 mg, 0.357 mmol) under nitrogen atmosphere. Then the mixture was stirred at 100° C. for 2 hours. The mixture was extracted by EA (25 mL×3). The combined organic layers were washed with brine (15 mL×3), dried over Na₂SO₄ and concentrated to give the crude product, which was purified by pre-HPLC to give the desired product as yellow solid (4.7 mg, 5.9%). ¹H NMR (400 MHz, DMSO-d₆) δ 8.81 (s, 1H), 8.32 (s, 1H), 7.60 (d, J=8.8 Hz, 1H), 7.38-7.29 (m, 2H), 7.15-7.07 (m, 2H), 6.91 (dd, J=8.8, 2.3 Hz, 1H), 6.80 (d, J=2.3 Hz, 1H), 6.00 (s, 1H), 2.94-2.79 (m, 4H), 1.25 (s, 9H). Mass (m/z): 337.4 [M+H]⁺.

### Compound 122

### 4-(2-aminoethyl)-7-((4-(4-(trifluoromethyl)piperidin-1-yl)phenyl)amino)-2H-chromen-2-one

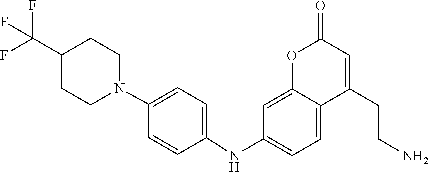 [SMILES: NCCc1cc(=O)oc2cc(Nc3ccc(N4CCC(C(F)(F)F)CC4)cc3)ccc12]

To a solution of 4-(2-(((allyloxy)carbonyl)amino)ethyl)-2-oxo-2H-chromen-7-yl trifluoromethanesulfonate (150 mg, 0.356 mmol) and 4-(4-(trifluoromethyl)piperidin-1-yl)aniline (130 mg, 0.534 mmol) in dioxane (5 mL) was added Pd₂(dba)₃ (16.3 mg, 0.018 mmol), Brettphos (19.1 mg, 0.0356 mmol) and Cs₂CO₃ (174 mg, 0.534 mmol) under nitrogen atmosphere. Then the mixture was stirred at 100° C. for 2 hours. The mixture was extracted by EA (25 mL×3). The combined organic layers were washed with brine (15 mL×3), dried over Na₂SO₄ and concentrated to give the crude product, which was purified by pre-HPLC to give the desired product as yellow solid (21.0 mg, 13.7%). ¹H NMR (400 MHz, DMSO-d₆) δ 8.59 (s, 1H), 7.56 (d, J=8.8 Hz, 1H), 7.06 (d, J=8.9 Hz, 2H), 6.98-6.91 (m, 2H), 6.80 (dd, J=8.8, 2.4 Hz, 1H), 6.64 (d, J=2.3 Hz, 1H), 5.93 (s, 1H), 3.69 (d, J=12.1 Hz, 2H), 2.81 (dd, J=21.2, 6.4 Hz, 4H), 2.70-2.59 (m, 4H), 1.86 (d, J=12.6 Hz, 2H), 1.61-1.46 (m, 2H). Mass (m/z): 432.4 [M+H]⁺.

### Compound 123

### 6-((4-(4-(trifluoromethyl)piperidin-1-yl)phenyl)amino)-2H-chromen-2-one

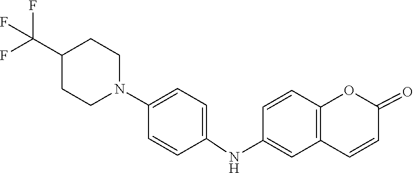 [SMILES: O=c1ccc2cc(Nc3ccc(N4CCC(C(F)(F)F)CC4)cc3)ccc2o1]

To a solution of 2-oxo-2H-chromen-6-yl trifluoromethanesulfonate (100 mg, 0.34 mmol) and 4-(4-(trifluoromethyl)piperidin-1-yl)aniline (91.2 mg, 0.374 mmol) in dioxane (5 mL) was added Pd₂(dba)₃ (15.6 mg, 0.017 mmol), Brettphos (18.3 mg, 0.034 mmol) and Cs₂CO₃ (222 mg, 0.68 mmol) under nitrogen atmosphere. Then the mixture was stirred at 100° C. overnight. The mixture was extracted by EA (25 mL×3). The combined organic layers were washed with brine (15 mL×3), dried over Na₂SO₄ and concentrated to give the crude product, which was purified by pre-HPLC to give the desired product as yellow solid (22.0 mg, 15.2%). ¹H NMR (400 MHz, DMSO-d₆) δ 7.94 (d, J=9.9 Hz, 2H), 7.21 (d, J=8.8 Hz, 1H), 7.16-7.07 (m, 2H), 7.03-6.95 (m, 2H), 6.93-6.85 (m, 2H), 6.37 (d, J=9.5 Hz, 1H), 3.61 (d, J=12.3 Hz, 2H), 2.59 (dd, J=12.3, 2.5 Hz, 2H), 2.43-2.39 (m, 1H), 1.85 (d, J=12.7 Hz, 2H), 1.54 (qd, J=12.5, 4.1 Hz, 2H). Mass (m/z): 389.3 [M+H]⁺.

### Compound 124

### 3-(2-aminoethyl)-7-((4-(tert-butyl)phenyl)amino)-2H-chromen-2-one

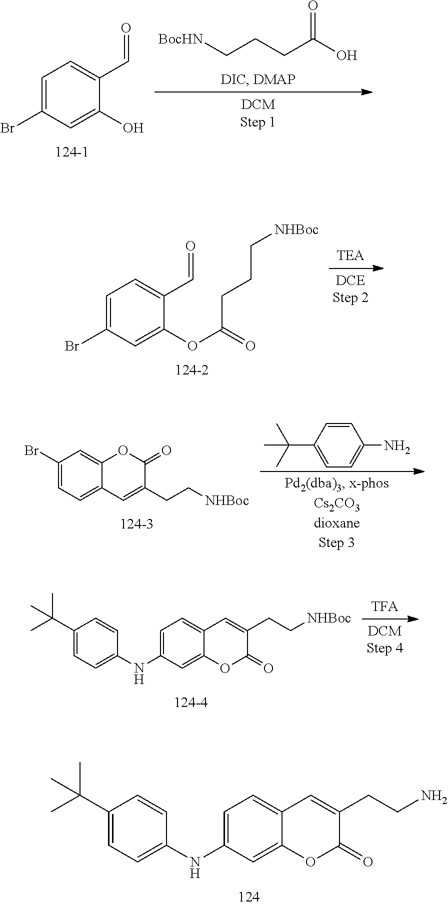

### Step 1. Preparation of 5-bromo-2-formylphenyl 4-((tert-butoxycarbonyl) amino)butanoate (124-2)

To a solution of 4-bromo-2-hydroxybenzaldehyde (3.5 g, 17.4 mmol) in DCM (50 mL) was added 4-((tert-butoxycarbonyl)amino)butanoic acid (4.24 g, 20.9 mmol), DIC (2.85 g, 22.6 mmol) and DMAP (637 mg, 5.22 mmol). Then the mixture was stirred at room temperature overnight. The mixture was diluted with water and extracted by DCM (25 mL×3). The combined organic layers were washed with brine (15 mL×3), dried over Na₂SO₄ and concentrated to give the crude product which was purified by Column chromatography to give the desired product 2.1 g as yellow solid. Mass (m/z): 386.3 [M+H]⁺.

### Step 2. Preparation of tert-butyl (2-(7-bromo-2-oxo-2H-chromen-3-yl)ethyl)carbamate (124-3)

To a solution of 5-bromo-2-formylphenyl 4-((tert-butoxycarbonyl) amino)butanoate (2.1 g, 5.44 mmol) in DCE (20 mL) was added TEA (1.1 g, 10.9 mmol), the mixture was reflux for 3 hours. Then it was diluted with water and extracted by EA (25 mL×3). The combined organic layers were washed with brine (15 mL×3), dried over Na₂SO₄ and concentrated to give the crude product which was purified by Column chromatography to give the desired product 300 mg as white solid. Mass (m/z): 369.3[M+H]⁺.

### Step 3. Preparation of Preparation of tert-butyl (2-(7-((4-(tert-butyl) phenyl)amino)-2-oxo-2H-chromen-3-yl)ethyl)carbamate (124-4)

To a solution of tert-butyl (2-(7-bromo-2-oxo-2H-chromen-3-yl) ethyl)carbamate (100 mg, 0.272 mmol) and 4-(tert-butyl)aniline (57 mg, 0.326 mmol) in dioxane (5 mL) was added Pd₂(dba)₃ (2.5 mg, 0.00272 mmol), X-phos (6.5 mg, 0.0136 mmol) and Cs₂CO₃ (133 mg, 0.408 mmol) under nitrogen atmosphere. Then the mixture was stirred at 100° C. overnight. The mixture was extracted by EA (25 mL×3). The combined organic layers were washed with brine (15 mL×3), dried over Na₂SO₄ and concentrated to give the crude product, which was purified by TLC to give the desired product as yellow solid 50 mg. Mass (m/z): 437.6 [M+H]⁺.

### Step 4. Preparation of 3-(2-aminoethyl)-7-((4-(tert-butyl)phenyl)amino)-2H-chromen-2-one (124)

To a solution of tert-butyl(2-(7-((4-(tert-butyl)phenyl)amino)-2-oxo-2H-chromen-3-yl)ethyl)carbamate (50 mg, 0.114 mmol) in DCM (9 mL) was added 2,2,2-trifluoroacetic acid (3 mL). Then the mixture was concentrated to give the crude product, which was purified by pre-HPLC to give the desired product as yellow solid (12.5 mg, 32.9%). ¹H NMR (400 MHz, DMSO-d₆) δ 8.81 (s, 1H), 8.32 (s, 1H), 7.60 (d, J=8.8 Hz, 1H), 7.38-7.29 (m, 2H), 7.15-7.07 (m, 2H), 6.91 (dd, J=8.8, 2.3 Hz, 1H), 6.80 (d, J=2.3 Hz, 1H), 6.00 (s, 1H), 2.94-2.79 (m, 4H), 1.25 (s, 9H). Mass (m/z): 337.2 [M+H]⁺.

### Compound 125

### 3-(2-aminoethyl)-7-((4-(4-(trifluoromethyl)piperidin-1-yl)phenyl) amino)-2H-chromen-2-one

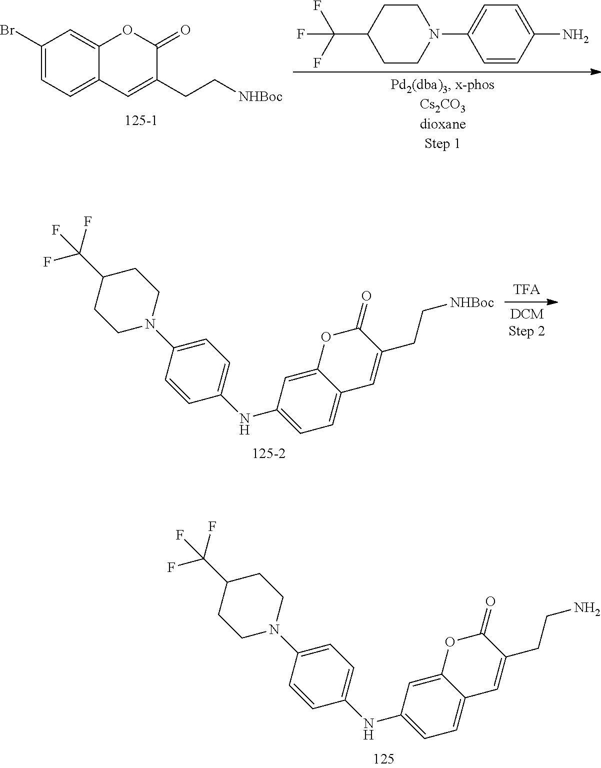 [SMILES: CC(C)(C)OC(=O)NCCc1cc2ccc(Br)cc2oc1=O ; Nc1ccc(N2CCC(C(F)(F)F)CC2)cc1 ; CC(C)(C)OC(=O)NCCc1cc2ccc(Nc3ccc(N4CCC(C(F)(F)F)CC4)cc3)cc2oc1=O ; NCCc1cc2ccc(Nc3ccc(N4CCC(C(F)(F)F)CC4)cc3)cc2oc1=O]

### Step 1. Preparation of tert-butyl(2-(2-oxo-7-((4-(4-(trifluoromethyl)piperidin-1-yl)phenyl)amino) 2H-chromen-3-yl)ethyl)carbamate (125-2)

To a solution of tert-butyl (2-(7-bromo-2-oxo-2H-chromen-3-yl)ethyl)carbamate (85 mg, 0.231 mmol) and 4-(4-(trifluoromethyl)piperidin-1-yl)aniline (67.6 mg, 0.277 mmol) in DMF (5 mL) was added Pd₂(dba)₃ (2.5 mg, 0.00272 mmol), X-phos (6.6 mg, 0.0139 mmol) and Cs₂CO₃ (135 mg, 0.416 mmol) under nitrogen atmosphere. Then the mixture was stirred at 100° C. overnight. The mixture was extracted by EA (25 mL×3). The combined organic layers were washed with brine (15 mL×3), dried over Na₂SO₄ and concentrated to give the crude product, which was purified by TLC to give the desired product as yellow solid (40 mg). Mass (m/z): 532.6 [M+H]⁺.

### Step 2. Preparation of 3-(2-aminoethyl)-7-((4-(4-(trifluoromethyl) piperidin-1-yl)phenyl)amino)-2H-chromen-2-one (125)

To a solution of tert-butyl(2-(2-oxo-7-((4-(4-(trifluoromethyl) piperidin-1-yl)phenyl)amino)-2H-chromen-3-yl)ethyl)carbamate (40 mg, 0.0752 mmol) in DCM (9 mL) was added 2,2,2-trifluoroacetic acid (3 mL). Then the mixture was concentrated to give the crude product, which was purified by pre-HPLC to give the desired product as yellow solid (5 mg, 15.6%). H NMR (400 MHz, DMSO-d₆) δ 8.56 (s, 1H), 8.34 (s, 1H), 7.65 (s, 1H), 7.36 (d, J=8.7 Hz, 1H), 7.05 (d, J=9.0 Hz, 2H), 6.94 (d, J=9.0 Hz, 2H), 6.78 (dd, J=8.6, 2.2 Hz, 1H), 6.65 (d, J=2.2 Hz, 1H), 3.68 (dd, J=12.1, 3.1 Hz, 3H), 2.86 (t, J=7.0 Hz, 2H), 2.77-2.51 (m, 7H), 1.86 (d, J=13.0 Hz, 3H), 1.55 (td, J=12.5, 4.1 Hz, 2H). Mass (m/z): 432.2 [M+H]⁺.

### Compound 126

### 4,6-dimethyl-7-((4-(4-(trifluoromethyl)piperidin-1-yl)phenyl)amino)-2H-chromen-2-one

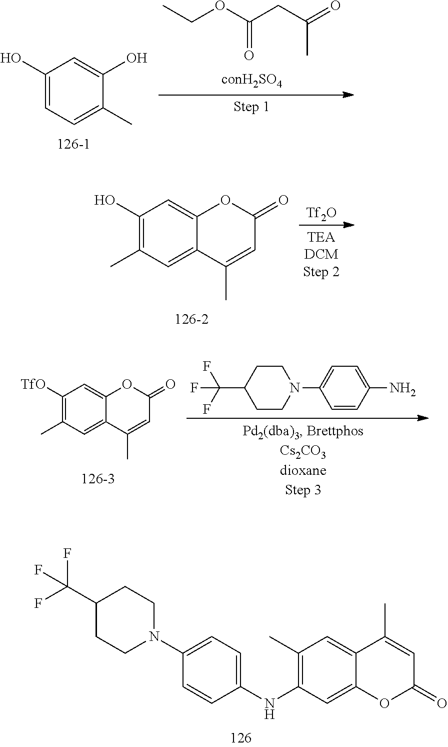

### Step 1. Preparation of 7-hydroxy-4,6-dimethyl-2H-chromen-2-one

To a solution of 4-methylbenzene-1,3-diol (2.0 g, 16.1 mmol) in ethyl 3-oxobutanoate (3 mL) was added con·H₂SO₄ (5 mL) at 0° C. Then the mixture was stirred at 0° C. for 5 hours. The mixture was filtered and washed with water till PH to 7-8 and dried to give the desired product 1.1 g as grey solid. Mass (m/z): 191.2 [M+H]⁺.

### Step 2. Preparation of 4,6-dimethyl-2-oxo-2H-chromen-7-yl trifluoromethanesulfonate

To a solution of 7-hydroxy-4,6-dimethyl-2H-chromen-2-one (200 mg, 1.05 mmol) in DCM (10 mL) was added trifluoromethanesulfonic anhydride (356 mg, 1.26 mmol) and TEA (159 mg, 1.58 mmol) at 0° C. Then the mixture was stirred at room temperature for 3 hours. The mixture was diluted with water and extracted by DCM (25 mL×3). The combined organic layers were washed with brine (15 mL×3), dried over Na₂SO₄ and concentrated to give the crude product which was purified by Column chromatography to give the desired product 200 mg as yellow solid. Mass (m/z): 323.3 [M+H]⁺.

### Step 3. Preparation of 4,6-dimethyl-7-((4-(4-(trifluoromethyl)piperidin-1-yl)phenyl)amino)-2H-chromen-2-one (126)

To a solution of 4,6-dimethyl-2-oxo-2H-chromen-7-yl trifluoromethanesulfonate (87 mg, 0.270 mmol) and 4-(4-(trifluoromethyl) piperidin-1-yl)aniline (60 mg, 0.246 mmol) in dioxane (3 mL) was added Pd₂(dba)₃ (11.3 mg, 0.0123 mmol), Brettphos (13.2 mg, 0.0246 mmol) and Cs₂CO₃ (120 mg, 0.369 mmol) under nitrogen atmosphere. Then the mixture was stirred at 100° C. overnight. The mixture was extracted by EA (25 mL×3). The combined organic layers were washed with brine (15 mL×3), dried over Na₂SO₄ and concentrated to give the crude product, which was purified by pre-HPLC to give the desired product as yellow solid (34.5 mg, 33.8%). ¹H NMR (400 MHz, DMSO-d₆) δ 7.62 (s, 1H), 7.45 (s, 1H), 7.17-7.08 (m, 2H), 7.06-6.92 (m, 2H), 6.57 (s, 1H), 5.98 (d, J=1.3 Hz, 1H), 3.75 (d, J=12.4 Hz, 2H), 2.72 (dd, J=12.3, 2.5 Hz, 2H), 2.35 (d, J=1.2 Hz, 3H), 2.29 (s, 3H), 1.96-1.83 (m, 2H), 1.58 (qd, J=12.5, 4.0 Hz, 2H). Mass (m/z): 417.4 [M+H]⁺.

### Compound 127

### 1-methyl-6-((4-(4-(trifluoromethyl)piperidin-1-yl)phenyl)amino)-3,4-dihydroquinolin-2(1H)-one

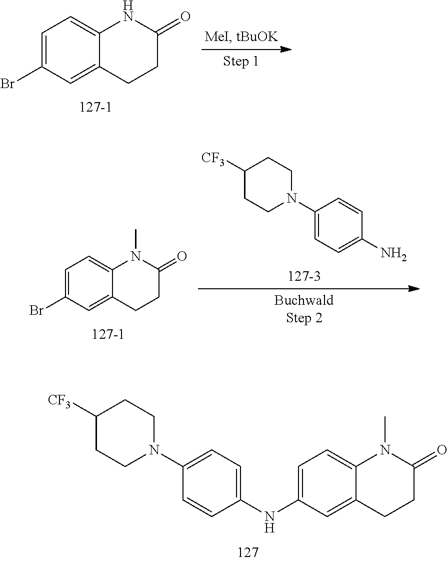 [SMILES: O=C1CCc2cc(Br)ccc2N1 ; CN1C(=O)CCc2cc(Br)ccc21 ; Nc1ccc(N2CCC(C(F)(F)F)CC2)cc1 ; CN1C(=O)CCc2cc(Nc3ccc(N4CCC(C(F)(F)F)CC4)cc3)ccc21]

### Step 1. Preparation of 6-bromo-1-methyl-3,4-dihydroquinolin-2(1H)-one (127-2)

To a solution of 6-bromo-3,4-dihydroquinolin-2(1H)-one 127-1 (500 mg, 2.21 mmol) and t-BuOK (496 mg, 4.42 mmol) in DMF (10 mL) was added Mel (408 mg, 2.88 mmol). Then the mixture was stirred at room temperature for overnight. The mixture was poured into H₂O and extracted with EA (30 mL*3). The organic layer was washed with brine, dried over Na₂SO₄, filtered and concentrated, the residue was purified by silica gel chromatography with EA/PE (20:1 to 5:1) to give 6-bromo-1-methyl-3,4-dihydroquinolin-2(1H)-one 127-2 (493 mg, 93% yield) as a yellow solid. MS (ESI) m/z 239.8, 241.8 [M+H]⁺.

### Step 2. Preparation of 1-methyl-6-((4-(4-(trifluoromethyl) piperidin-1-yl) phenyl)amino)-3,4-dihydroquinolin-2(1H)-one (127)

A mixture solution of 4-(4-(trifluoromethyl)piperidin-1-yl)aniline 127-3 (300 mg, 1.23 mmol), 6-bromo-1-methyl-3,4-dihydroquinolin-2(1H)-one 127-2 (295 mg, 1.23 mmol), Ruphos (115 mg, 0.25 mmol), Pd₂(dba)₃ (112 mg, 0.12 mmol) and Cs₂CO₃ (600 mg, 1.84 mmol) in 1,4-dioxane (10 mL) was stirred at 100° C. under N₂ atmosphere for 12 hrs. The mixture was concentrated and the residue was purified by pre-HPLC to give compound 127 as a yellow solid (25.3 mg, 5%). Mass (m/z): 404.2 [M+H]⁺. ¹H NMR (400 MHz, CD₃OD) δ 6.98 (d, J=9.0 Hz, 2H), 6.92 (dd, J=8.8, 4.2 Hz, 3H), 6.89-6.84 (m, 1H), 6.81 (d, J=2.5 Hz, 1H), 3.57 (dd, J=13.1, 0.6 Hz, 2H), 3.28 (s, 3H), 2.78 (dd, J=8.5, 6.2 Hz, 2H), 2.63 (td, J=12.2, 1.7 Hz, 2H), 2.54 (dd, J=8.5, 6.2 Hz, 2H), 2.33-2.15 (m, 1H), 1.99-1.89 (m, 2H), 1.77-1.64 (m, 2H).

### Compound 128

### 1-methyl-6-((4-(4-(trifluoromethyl)piperidin-1-yl)phenyl)amino)-1,4-dihydro-2H-benzo[d][1,3]oxazin-2-one

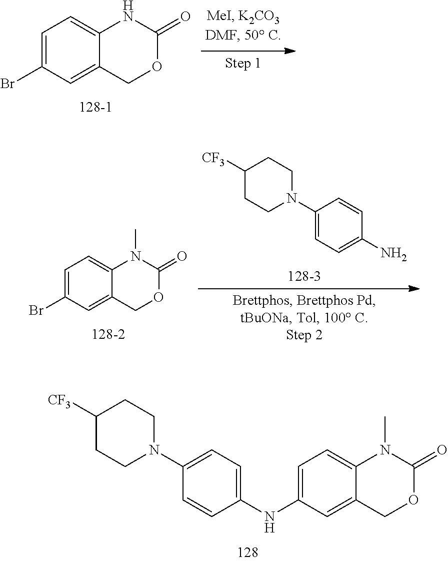 [SMILES: O=C1Nc2ccc(Br)cc2CO1 ; CN1C(=O)OCc2cc(Br)ccc21 ; Nc1ccc(N2CCC(C(F)(F)F)CC2)cc1 ; CN1C(=O)OCc2cc(Nc3ccc(N4CCC(C(F)(F)F)CC4)cc3)ccc21]

### Step 1. Preparation of 6-bromo-1-methyl-1,4-dihydro-2H-benzo[d][1,3]oxazin-2-one (128-2)

To a solution of compound 411-1 (300 mg, 1.316 mmol) in DMF (10 mL) was added K₂CO₃ (363.63 mg, 2.631 mmol) and Mel (280 mg, 1.973 mmol) at 50° C. Then the mixture was stirred at 50° C. for 16 hrs. After cooling to rt, the reaction solution was washed with water, extracted with ethyl acetate (20 mL*3). The organic layers were combined and concentrated under vacuum, the residue was purified through silica gel chromatography (PE:EA=5:1) to give 6-bromo-1-methyl-1,4-dihydro-2H-benzo[d][1,3] oxazin-2-one as white solid (150 mg, 37.68%). Mass (m/z): 377.3 [M+H]⁺.

### Step 2. Preparation of 1-methyl-6-((4-(4-(trifluoromethyl)piperidin-1-yl) phenyl)amino)-1,4-dihydro-2H-benzo[d][1,3]oxazin-2-one (128)

A mixture of 6-bromo-1-methyl-1,4-dihydro-2H-benzo[d][1,3]oxazin-2-one (150 mg, 0.6197 mmol), 4-(4-(trifluoromethyl)piperidin-1-yl) aniline (151.37 mg, 0.6197 mmol), NaOtBu (119.11 mg, 1.2394 mmol), Brettphos (33.26 mg, 0.062 mmol), Brettphos-Pd-G1 (49.44 mg, 0.062 mmol) in toluene (10 mL) was stirred 10 hrs at 100° C. under N₂ atmosphere. After cooling to rt, the reaction solution was washed with water, extracted with ethyl acetate (20 mL*3), the organic layers were combined and concentrated under vacuum, the residue was purified by prep-HPLC(column-Xbridge-C18 150×21.2 mm, 5 um; Mobile phase: ACN-H₂O (0.1% FA), 30%-35%) to afford compound 128 as yellow solid. (12.2 mg, 4.71%). Mass (m/z): 406 [M+H]⁺. ¹H NMR (400 MHz, CD₃OD) δ 7.07-6.93 (m, 6H), 6.86 (d, J=2.1 Hz, 1H), 5.17 (s, 2H), 3.63 (d, J=12.2 Hz, 2H), 3.34 (s, 3H), 2.68 (td, J=12.3, 2.2 Hz, 2H), 2.34-2.25 (m, 1H), 1.99 (d, J=13.3 Hz, 2H), 1.79-1.69 (m, 2H).

### Compound 129

### 7-((4-(4-methoxy-4-(trifluoromethyl)piperidin-1-yl)-2-methylphenyl) amino)-4-methyl-2H-benzo[b][1,4]oxazin-3(4H)-one

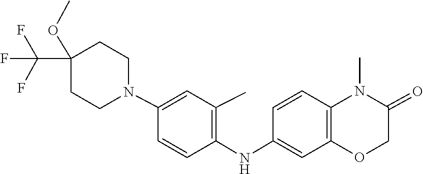 [SMILES: COC1(C(F)(F)F)CCN(c2ccc(Nc3ccc4c(c3)OCC(=O)N4C)c(C)c2)CC1]

To a solution of 7-bromo-4-methyl-2H-benzo[b][1,4]oxazin-3(4H)-one (138 mg, 0.57 mmol) and 4-(4-methoxy-4-(trifluoromethyl) piperidin-1-yl)-2-methylaniline (150 mg, 0.52 mmol) in 1,4-dioxane (2 mL) was added Pd₂(dba)₃ (47 mg, 0.05 mmol), Xphos (49 mg, 0.10 mmol) and Cs₂CO₃ (339 mg, 1.04 mmol) under nitrogen atmosphere. The mixture was stirred at 100° C. for 12 h, the mixture was purified by prep TLC then purified by prep HPLC to give 7-((4-(4-methoxy-4-(trifluoromethyl)piperidin-1-yl)-2-methylphenyl)amino)-4-methyl-2H-be nzo[b][1,4]oxazin-3(4H)-one (57.9 mg, 29.1%) as a red solid. Mass (m/z): 450.4 [M+H]⁺. ¹H NMR (400 MHz, DMSO-d₆) δ 7.21 (s, 1H), 7.07-6.86 (m, 4H), 6.83-6.75 (m, 1H), 6.39-6.32 (m, 1H), 6.27 (d, J=2.5 Hz, 1H), 4.54 (s, 2H), 3.58-3.50 (m, 2H), 3.40 (s, 3H), 3.20 (s, 3H), 2.92-2.73 (m, 2H), 2.49-2.44 (m, 1H), 2.12 (s, 3H), 2.06-1.95 (m, 2H), 1.88-1.76 (m, 2H).

### Compound 130

### 7-((4-(4-hydroxy-4-(trifluoromethyl)piperidin-1-yl)-2-methylphenyl)amino)-4-methyl-2H-benzo[b][1,4]oxazin-3(4H)-one

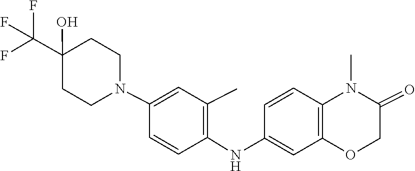 [SMILES: Cc1cc(N2CCC(O)(C(F)(F)F)CC2)ccc1Nc1ccc2c(c1)OCC(=O)N2C]

To a solution of 7-bromo-4-methyl-2H-benzo[b][1,4]oxazin-3(4H)-one (48 mg, 0.20 mmol) and 1-(4-amino-3-methylphenyl)-4-(trifluoromethyl)piperidin-4-ol (50 mg, 0.18 mmol) in 1,4-dioxane (1 mL) was added Pd₂(dba)₃ (17 mg, 0.02 mmol), Xphos (17 mg, 0.04 mmol) and Cs₂CO₃ (119 mg, 0.36 mmol) under nitrogen atmosphere. The mixture was stirred at 100° C. for 12 h, the mixture was purified by prep TLC then purified by prep HPLC to give 7-((4-(4-hydroxy-4-(trifluoromethyl)piperidin-1-yl)-2-methylphenyl)amino)-4-methyl-2H-be nzo[b][1,4]oxazin-3(4H)-one (20.5 mg, 29.4%) as a pink solid. Mass (m/z): 436.4 [M+H]⁺. ¹H NMR (400 MHz, DMSO-d₆) δ 7.20 (s, 1H), 6.98 (d, J=8.6 Hz, 1H), 6.91 (d, J=8.7 Hz, 1H), 6.87 (d, J=2.8 Hz, 1H), 6.82-6.74 (m, 1H), 6.39-6.31 (m, 1H), 6.27 (d, J=2.4 Hz, 1H), 5.95 (s, 1H), 4.53 (s, 2H), 3.57-3.50 (m, 2H), 3.20 (s, 3H), 3.01-2.81 (m, 2H), 2.12 (s, 3H), 1.85-1.68 (m, 4H).

### Compound 131

### 7-((5-methoxy-6-(4-(trifluoromethyl)piperidin-1-yl)pyridin-3-yl)amino)-2H-benzo[b][1,4]oxazin-3(4H)-one

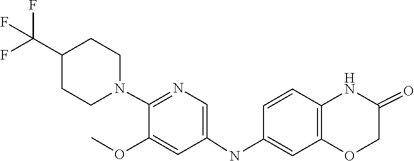

To a solution of 7-bromo-2H-benzo[b][1,4]oxazin-3(4H)-one (66 mg, 0.291 mmol) and 5-methoxy-6-(4-(trifluoromethyl) piperidin-1-yl) pyridin-3-amine (80 mg, 0.291 mmol) in t-BuOH (5 mL) was added Pd₂(dba)₃ (13.3 mg, 0.0146 mmol), Brettphos (15.6 mg, 0.0291 mmol) and Cs₂CO₃ (142 mg, 0.437 mmol) under nitrogen atmosphere. Then the mixture was stirred at 100° C. overnight. The mixture was extracted by EA (25 mL×3). The combined organic layers were washed with brine (15 mL×3), dried over Na₂SO₄ and concentrated to give the crude product, which was purified by pre-HPLC to give the desired product as pink solid (51.9 mg, 42.3%). ¹H NMR (400 MHz, DMSO) δ 10.50 (s, 1H), 7.87 (s, 1H), 7.59 (d, J=2.2 Hz, 1H), 6.95 (t, J=9.7 Hz, 1H), 6.74 (d, J=8.4 Hz, 1H), 6.62-6.57 (m, 1H), 6.55 (d, J=2.3 Hz, 1H), 4.50 (s, 2H), 3.85-3.68 (m, 5H), 2.73-2.56 (m, 2H), 2.43 (s, 1H), 1.84 (d, J=11.0 Hz, 2H), 1.56 (qd, J=12.5, 3.9 Hz, 2H). Mass (m/z): 423.2 [M+H]⁺.

### Compound 132

### 7-((4-(2,6-dimethylmorpholino)-3-methylphenyl)amino)-5-fluoro-4-methyl-2H-benzo[b][1,4]oxazin-3(4H)-one

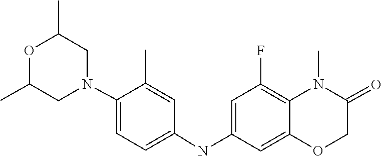

To a solution of 7-bromo-5-fluoro-4-methyl-2H-benzo[b][1,4]oxazin-3(4H)-one (70.7 mg, 0.273 mmol) and 4-(2,6-dimethylmorpholino)-3-methylaniline (60 mg, 0.273 mmol) in t-BuOH (5 mL) was added Pd₂(dba)₃ (12.5 mg, 0.0136 mmol), X-phos (13 mg, 0.0273 mmol) and Cs₂CO₃ (133 mg, 0.41 mmol) under a nitrogen atmosphere. Then the mixture was stirred at 100° C. overnight. The mixture was extracted by EA (25 mL×3). The combined organic layers were washed with brine (15 mL×3), dried over Na₂SO₄ and concentrated to give the crude product, which was purified by pre-HPLC to give the desired product as a pink solid (12.6 mg, 11.6%). ¹H NMR (400 MHz, DMSO) δ 8.17 (s, 1H), 6.96 (d, J=9.4 Hz, 1H), 6.89 (d, J=7.0 Hz, 2H), 6.55-6.37 (m, 2H), 4.56 (s, 2H), 3.77-3.66 (m, 2H), 3.30 (d, J=5.5 Hz, 2H), 2.87 (d, J=10.8 Hz, 2H), 2.37-2.24 (m, 3H), 2.22 (s, 3H), 1.12 (dd, J=14.3, 10.1 Hz, 6H). Mass (m/z): 400.2 [M+H]⁺.

### Compound 133

### N-(4-(2,6-dimethylmorpholino)phenyl)-1-methyl-1H-benzo[d][1,2,3]triazol-6-amine

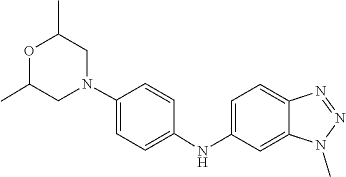 [SMILES: CC1CN(c2ccc(Nc3ccc4nnn(C)c4c3)cc2)CC(C)O1]

To a solution of 4-(2,6-dimethylmorpholin-4-yl) aniline (107 mg, 0.52 mmol), 6-bromo-1-methyl-1,2,3-benzotriazole (121 mg, 0.57 mmol) and t-BuONa (100 mg, 1.1 mmol) in toluene (2 mL) was added BrettPhos Pd G3 (47 mg, 0.052 mmol). The mixture was heated to 100° C. and stirred under nitrogen for 18 h. The resulting mixture was concentrated and the residue was purified by column chromatography (EA:PE=1:1) to give the product (27.5 mg, 15.56%) as a white solid. MS (ESI) m/z: 338 [M+H]⁺. ¹H NMR (400 MHz, DMSO-d₆) δ 8.28 (s, 1H), 7.72 (d, J=9.2 Hz, 1H), 7.14 (d, J=9.2 Hz, 2H), 6.98-6.94 (m, 4H), 4.09 (s, 2H), 3.72-3.68 (m, 2H), 3.51 (d, J=10.4 Hz, 2H), 2.25-2.20 (m, 2H), 1.15 (d, J=6.4 Hz, 6H).

### Compound 134

### 1-hydroxy-5-oxo-N-(4-((4-(4-(trifluoromethyl)piperidin-1-yl)phenyl) amino)benzyl)pyrrolidine-3-carboxamide

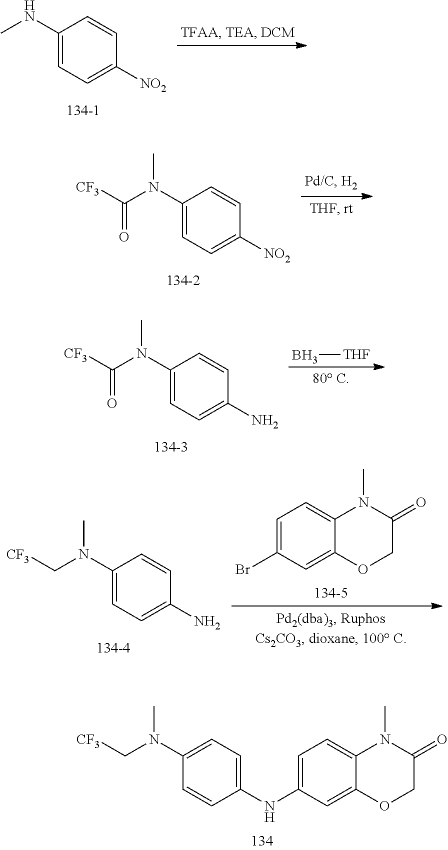

### Step 1. Preparation of 2,2,2-trifluoro-N-methyl-N-(4-nitrophenyl) acetamide (134-2)

A mixture solution of N-methyl-4-nitroaniline (1 g, 6.58 mmol), Trifluoroacetic anhydride (TFAA, 1.52 g, 7.24 mmol), TEA (1.33 g, 13.16 mmol) in DCM (20 mL) was stirred at 25° C. for 12 hrs. The mixture was concentrated and the residue was purified by flash chromatography, eluting with PE:EA=10:1 to 1:1 to give 2,2,2-trifluoro-N-methyl-N-(4-nitrophenyl)acetamide as a yellow oil (1.5 g, 91.2%). Mass (m/z): 249.1 [M+H]⁺.

### Step 2. Preparation of N-(4-aminophenyl)-2,2,2-trifluoro-N-methylacetamide (134-3)

A mixture solution of 2,2,2-trifluoro-N-methyl-N-(4-nitrophenyl) acetamide 134-2 (1.5 g, 6.05 mmol), Pd/C (0.4 g) in THF (20 mL) was stirred under 1 atm of H₂ atmosphere at 25° C. for 2 hrs. After filtration, the filtrate was removed under vacuum and the residue was purified by flash, eluting with PE:EA=10:1 to 1:1 to give N-(4-aminophenyl)-2,2,2-trifluoro-N-methylacetamide 134-3 as yellow solid. (1.2 g, 91.7%). Mass (m/z): 218.07 [M+H]⁺.

### Step 3. Preparation of N¹-methyl-N¹-(2,2,2-trifluoroethyl)benzene-1,4-diamine (417-4)

A solution of N-(4-aminophenyl)-2,2,2-trifluoro-N-methylacetamide 134-3 (0.5 g, 2.29 mmol) in BH₃-THF (5 mL, 1.0 mol/L) and THF (2 mL) was stirred at 80° C. for 12 hrs. Quenched with MeOH (30 mL), concentrated, to the residue was added H₂O (30 mL), extracted with EA (20 mL*3), the organic phase was concentrated under vacuum, the residue was purified by flash, eluting with PE:EA=10:1 to 1:1 to give N¹-methyl-N¹-(2,2,2-trifluoroethyl)benzene-1,4-diamine 134-4 as yellow solid. (0.16 g, 34.1%). Mass (m/z): 205.0 [M+H]⁺.

### Step 4. Preparation of 1-hydroxy-5-oxo-N-(4-((4-(4-(trifluoromethyl) piperidin-1-yl)phenyl)amino)benzyl)pyrrolidine-3-carboxamide (134)

A mixture solution of N¹-methyl-N¹-(2,2,2-trifluoroethyl)benzene-1,4-diamine 134-4 (0.16 g, 0.78 mmol), 7-bromo-4-methyl-2H-benzo[b][1,4]oxazin-3(4H)-one (134-5) (0.2 g, 0.83 mmol), Ruphos (73 mg, 0.16 mmol), Pd₂(dba)₃ (72 mg, 0.08 mmol) and Cs₂CO₃ (0.76 g, 2.34 mmol) in 1,4-dioxane (10 mL) was stirred at 100° C. under N₂ atmosphere for 12 hrs. The mixture was concentrated and the residue was purified by prep-HPLC to give compound 134 as a yellow solid (0.134 g, 47.1%). Mass (m/z): 365.7 [M+H]⁺. ¹H NMR (400 MHz, DMSO-d₆) δ 7.72 (s, 1H), 6.96 (t, J=8.6 Hz, 3H), 6.81 (d, J=8.0 Hz, 2H), 6.57 (dd, J=8.8, 2.4 Hz, 1H), 6.49 (d, J=2.4 Hz, 1H), 4.55 (s, 2H), 4.13 (q, J=9.6 Hz, 2H), 3.22 (s, 3H), 2.96 (s, 3H).

### Compound 135

### 7-((4-(tert-butyl)phenyl)amino)-2H-benzo[b][1,4]oxazin-3(4H)-one

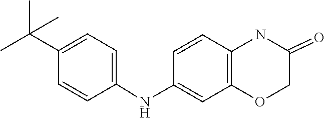

To a solution of 7-bromo-2H-benzo[b][1,4]oxazin-3(4H)-one (333 mg, 1.48 mmol) and 4-(tert-butyl)aniline (60 mg, 0.26 mmol) in 1,4-dioxane (2 mL) was added Pd₂(dba)₃ (122 mg, 0.13 mmol), Brettphos (144 mg, 0.26 mmol) and Cs₂CO₃ (875 mg, 2.68 mmol) under nitrogen atmosphere. The mixture was stirred at 100° C. for 12 h, the mixture was purified by prep TLC then purified by prep HPLC to give 7-((4-(tert-butyl)phenyl)amino)-2H-benzo[b][1,4]oxazin-3(4H)-one (32 mg, 31%) as a yellow solid. Mass (m/z): 297.3 [M+H]⁺. ¹H NMR (400 MHz, DMSO) δ 10.50 (s, 1H), 7.93 (s, 1H), 7.27-7.19 (m, 2H), 6.98-6.90 (m, 2H), 6.75 (d, J=8.4 Hz, 1H), 6.65 (dd, J=8.4, 2.4 Hz, 1H), 6.60 (d, J=2.3 Hz, 1H), 4.50 (s, 2H), 1.24 (s, 9H).

### Compound 136

### 7-((3,5-difluoro-4-(4-(trifluoromethyl)piperidin-1-yl)phenyl)amino)-2H-benzo[b][1,4]oxazin-3(4H)-one

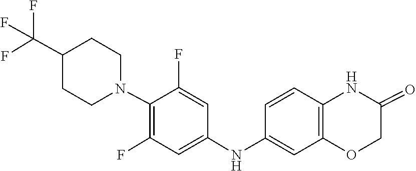 [SMILES: O=C1COc2cc(Nc3cc(F)c(N4CCC(C(F)(F)F)CC4)c(F)c3)ccc2N1]

To a solution of 7-bromo-2H-benzo[b][1,4]oxazin-3(4H)-one (151 mg, 0.67 mmol) and 3,5-difluoro-4-(4-(trifluoromethyl)piperidin-1-yl)aniline (170 mg, 0.61 mmol) in 1,4-dioxane (2 mL) was added Pd₂(dba)₃ (55 mg, 0.06 mmol), Brettphos (65 mg, 0.12 mmol) and Cs₂CO₃ (396 mg, 1.21 mmol) under nitrogen atmosphere. The mixture was stirred at 100° C. for 12 h, the mixture was purified by prep TLC then purified by prep HPLC to give 7-((3,5-difluoro-4-(4-(trifluoromethyl)piperidin-1-yl)phenyl) amino)-2H-benzo[b][1,4]oxazin-3(4H)-one (28 mg, 10.8%) as a white solid. Mass (m/z): 428.3 [M+H]⁺. ¹H NMR (400 MHz, DMSO-d₆) δ 10.61 (s, 1H), 8.31 (s, 1H), 6.82 (d, J=8.4 Hz, 1H), 6.71 (dd, J=8.4, 2.3 Hz, 1H), 6.66 (d, J=2.4 Hz, 1H), 6.58-6.46 (m, 2H), 4.54 (s, 2H), 3.11-2.92 (m, 4H), 2.49-2.35 (m, 1H), 1.88-1.79 (m, 2H), 1.61-1.46 (m, 2H).

### Compound 137

### 1-methyl-6-((4-(4-(trifluoromethyl)piperidin-1-yl)phenyl)amino)-3,4-dihydroquinazolin-2(1H)-one

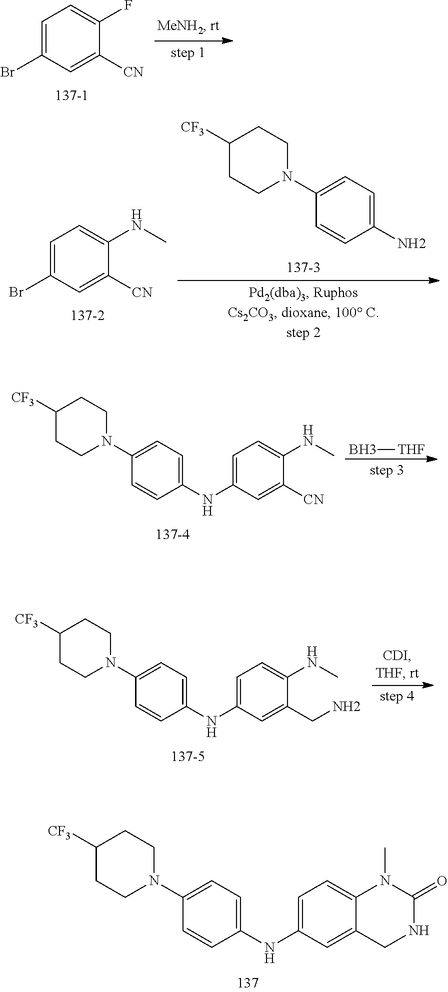

### Step 1. Preparation of 5-bromo-2-(methylamino)benzonitrile (137-2)

A mixture solution of 5-bromo-2-fluorobenzonitrile (5 g, 25 mmol) in MeNH₂ in MeOH (1 mol/L, 50 mL) was stirred at r.t. 2 hrs. Then the solvent was removed under reduced pressure, the residue was diluted with EA (50 mL), washed with water (50 mL×3), dried with Na₂SO₄, filtered and evaporated. The residue was purified by column chromatography to give 5-bromo-2-(methylamino)benzonitrile 137-2 (5 g, 94.76%).

### Step 2. Preparation of 2-(methylamino)-5-((4-(4-(trifluoromethyl)piperidin-1-yl)phenyl)amino)benzonitrile (137-4)

A mixture solution of 5-bromo-2-(methylamino)benzonitrile (200 mg, 0.95 mmol), 4-(4-(trifluoromethyl)piperidin-1-yl)aniline (255 mg, 1.04 mmol), Cs₂CO₃ (926 mg, 2.84 mmol), Ruphos (88 mg, 0.19 mmol) and Pd₂(dba)₃ (87 mg, 0.09 mmol) in dioxane was stirred at 100° C. for 16 hrs. Then the solvent was removed under vacuo, the residue was purified by silica gel column with PE:EA (3:1) to give 2-(methylamino)-5-((4-(4-(trifluoromethyl)piperidin-1-yl)phenyl)amino)benzonitrile 137-4 (300 mg, 84.56%). Mass (m/z): 374.7 [M+H]⁺.

### Step 3. Preparation of 2-(aminomethyl)-N¹-methyl-N4-(4-(4-(trifluoromethyl)piperidin-1-yl)phenyl)benzene-1,4-diamine (137-5)

A solution of 2-(methylamino)-5-((4-(4-(trifluoromethyl)piperidin-1-yl) phenyl)amino)benzonitrile (300 mg, 0.80 mmol) in BH₃ in THF (1M, 20 mL) was stirred at r.t. for 16 hrs. Then the solvent was removed under vacuo, the residue was diluted with EA (20 mL), washed with water (20 mL×3), dried with Na₂SO₄, filtered and evaporated. The residue was purified by column chromatography to give 137-5 (300 mg, 98.93%). Mass (m/z): 378.8 [M+H]⁺.

### Step 4. Preparation of 1-methyl-6-((4-(4-(trifluoromethyl)piperidin-1-yl) phenyl)amino)-3,4-dihydroquinazolin-2(1H)-one (137)

A solution of 2-(aminomethyl)-N¹-methyl-N4-(4-(4-(trifluoromethyl) piperidin-1-yl)phenyl)benzene-1,4-diamine (300 mg, 0.79 mmol) and CDI (141 mg, 0.87 mmol) in THE was stirred at r.t. for 16 hrs. Then the solvent was removed under vacuo, the residue was purified by prep-HPLC to give 1-methyl-6-((4-(4-(trifluoromethyl)piperidin-1-yl)phenyl)amino)-3,4-dihy droquinazolin-2(1H)-one compound 137 (90 mg, 28.07%). Mass (m/z): 404.7 [M+H]⁺. ¹H NMR (400 MHz, CD₃OD) δ 6.89-6.76 (m, 7H), 4.29 (s, 2H), 3.57 (s, 2H), 3.23 (s, 3H), 2.65 (s, 2H), 2.37-2.17 (m, 1H), 1.96 (d, J=12.1 Hz, 2H), 1.72 (dd, J=12.6, 3.5 Hz, 2H).

### Compound 138

### 7-((4-(2,6-dimethylmorpholino)-3-methylphenyl)amino)-2H-benzo[b][1,4]oxazin-3(4H)-one

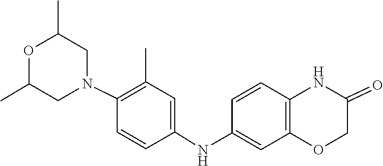 [SMILES: Cc1cc(Nc2ccc3c(c2)OCC(=O)N3)ccc1N1CC(C)OC(C)C1]

To a solution of 7-bromo-2H-benzo[b][1,4]oxazin-3(4H)-one (114 mg, 0.500 mmol) and 4-(2,6-dimethylmorpholino)-3-methylaniline (100 mg, 0.455 mmol) in t-BuOH (5 mL) was added Pd₂(dba)₃ (20.8 mg, 0.0228 mmol), Brettphos (24.4 mg, 0.0455 mmol) and Cs₂CO₃ (223 mg, 0.683 mmol) under nitrogen atmosphere. Then the mixture was stirred at 100° C. for 2 hours. The mixture was extracted by EA (25 mL×3). The combined organic layers were washed with brine (15 mL×3), dried over Na₂SO₄ and concentrated to give the crude product, which was purified by pre-HPLC to give the desired product as white solid (37.2 mg, 22.3%). ¹H NMR (400 MHz, DMSO-d₆) δ 10.48 (s, 1H), 7.79 (s, 1H), 6.90 (d, J=8.2 Hz, 1H), 6.85-6.76 (m, 2H), 6.73 (d, J=8.4 Hz, 1H), 6.62-6.50 (m, 2H), 4.49 (s, 2H), 3.78-3.60 (m, 2H), 2.81 (t, J=16.9 Hz, 2H), 2.37-2.22 (m, 2H), 2.20 (s, 3H), 1.10 (d, J=6.3 Hz, 6H). Mass (m/z): 368.2 [M+H]⁺.

### Compound 139

### 7-((4-(2,6-dimethylmorpholino)phenyl)amino)-2-methyl-2H-benzo[b][1,4]oxazin-3(4H)-one

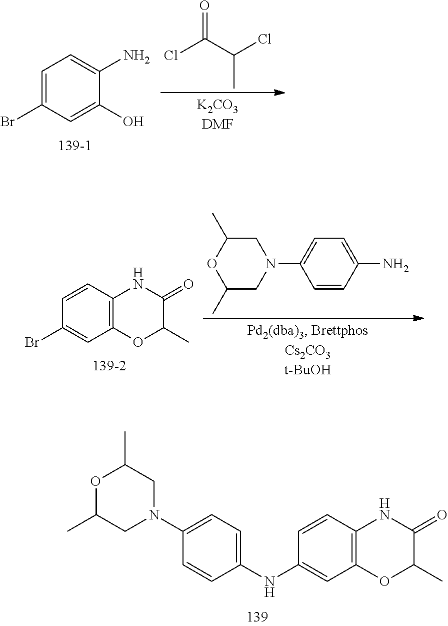

### Step 1. Preparation of 7-bromo-2-methyl-2H-benzo[b][1,4]oxazin-3(4H)-one (139-2)

To a solution of 2-amino-5-bromophenol (5.0 g, 26.6 mmol) in DMF (30 mL) was added K₂CO₃(7.34 g, 53.2 mmol), 2-chloropropanoyl chloride (3.72 g, 29.3 mmol) was added at 0° C. Then the mixture was stirred at r.t. for 0.5 hour then heated at 90° C. for 2 hours. The mixture was filtered and washed with water, dried over Na₂SO₄ and concentrated to give the desired product 4.1 g as grey solid. Mass (m/z): 242.1 [M+H]⁺.

### Step 2. Preparation of 7-((4-(2,6-dimethylmorpholino)phenyl)amino)-2-methyl-2H-benzo[b][1,4]oxazin-3(4H)-one (139)

To a solution of 7-bromo-2-methyl-2H-benzo[b][1,4]oxazin-3(4H)-one (129 mg, 0.534 mmol) and 4-(2,6-dimethylmorpholino)aniline (100 mg, 0.485 mmol) in t-BuOH (5 mL) was added Pd₂(dba)₃ (22.2 mg, 0.0243 mmol), Brettphos (26 mg, 0.0485 mmol) and Cs₂CO₃ (237 mg, 0.728 mmol) under nitrogen atmosphere. Then the mixture was stirred at 100° C. overnight. The mixture was extracted by EA (25 mL×3). The combined organic layers were washed with brine (15 mL×3), dried over Na₂SO₄ and concentrated to give the crude product, which was purified by pre-HPLC to give the desired product as gray solid (41.7 mg, 23.4%). ¹H NMR (400 MHz, DMSO-d₆) δ 10.45 (s, 1H), 7.69 (s, 1H), 6.90 (dd, J=29.6, 9.0 Hz, 4H), 6.71 (d, J=8.4 Hz, 1H), 6.58-6.42 (m, 2H), 4.93-4.87 (m, 1H), 3.67 (tdd, J=25.5, 14.8, 10.7 Hz, 2H), 3.43 (d, J=10.6 Hz, 2H), 2.27-2.10 (m, 2H), 1.58-1.55 (m, 3H), 1.14 (d, J=6.2 Hz, 6H). Mass (m/z): 368.2 [M+H]⁺.

### Compound 140

### 7-((4-(2,6-dimethylmorpholino)phenyl)amino)-2H-benzo[b][1,4]oxazin-3(4H)-one

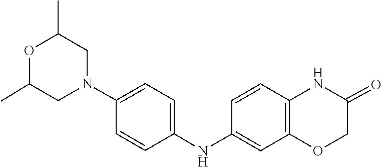 [SMILES: CC1CN(c2ccc(Nc3ccc4c(c3)OCC(=O)N4)cc2)CC(C)O1]

To a solution of 7-bromo-2H-benzo[b][1,4]oxazin-3(4H)-one (110.2 mg, 0.485 mmol) and 4-(2,6-dimethylmorpholino)aniline (100 mg, 0.485 mmol) in t-BuOH (5 mL) was added Pd₂(dba)₃ (22.2 mg, 0.0243 mmol), Brettphos (26 mg, 0.0485 mmol) and Cs₂CO₃ (237 mg, 0.728 mmol) under nitrogen atmosphere. Then the mixture was stirred at 100° C. overnight. The mixture was extracted by EA (25 mL×3). The combined organic layers were washed with brine (15 mL×3), dried over Na₂SO₄ and concentrated to give the crude product, which was purified by pre-HPLC to give the desired product as white solid (50 mg, 29.2%). ¹H NMR (400 MHz, DMSO-d₆) δ 10.45 (s, 1H), 7.69 (s, 1H), 6.90 (dd, J=29.6, 9.0 Hz, 4H), 6.71 (d, J=8.4 Hz, 1H), 6.58-6.42 (m, 2H), 4.48 (s, 2H), 3.67 (tdd, J=25.5, 14.8, 10.7 Hz, 2H), 3.43 (d, J=10.6 Hz, 2H), 2.27-2.10 (m, 2H), 1.14 (d, J=6.2 Hz, 6H). Mass (m/z): 354.2 [M+H]⁺.

### Compound 141 ((4-(2,6-dimethylmorpholino)-2-methylphenyl)amino)-5-fluoro-4-methyl-2H-benzo[b][1,4]oxazin-3(4H)-one

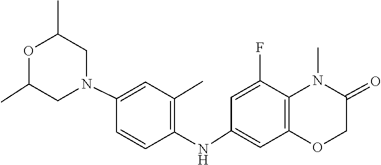 [SMILES: Cc1cc(N2CC(C)OC(C)C2)ccc1Nc1cc(F)c2c(c1)OCC(=O)N2C]

To a solution of 7-bromo-5-fluoro-4-methyl-2H-benzo[b][1,4]oxazin-3(4H)-one (90 mg, 0.347 mmol) and 4-(2,6-dimethylmorpholino)-2-methylaniline (77 mg, 0.347 mmol) in dioxane (5 mL) was added Pd₂(dba)₃ (16 mg, 0.0174 mmol), X-phos (16.6 mg, 0.0347 mmol) and Cs₂CO₃ (170 mg, 0.520 mmol) under nitrogen atmosphere. Then the mixture was stirred at 100° C. overnight. The mixture was extracted by EA (25 mL×3). The combined organic layers were washed with brine (15 mL×3), dried over Na₂SO₄ and concentrated to give the crude product, which was purified by pre-HPLC to give the desired product as pink solid (45 mg, 32.6%). ¹H NMR (400 MHz, DMSO-d₆) δ 7.55 (s, 1H), 6.99 (d, J=8.6 Hz, 1H), 6.89-6.72 (m, 2H), 6.16-6.03 (m, 2H), 4.52 (s, 2H), 3.74-3.63 (m, 2H), 3.53 (d, J=10.6 Hz, 2H), 3.27 (d, J=5.5 Hz, 3H), 2.23 (dd, J=21.8, 10.8 Hz, 2H), 2.10 (d, J=15.7 Hz, 3H), 1.15 (d, J=6.2 Hz, 6H). Mass (m/z): 400.2 [M+H]⁺.

### Compound 142

### 4-methyl-7-((5-methyl-6-(4-(trifluoromethyl)piperidin-1-yl)pyridin-3-yl) amino)-2H-benzo[b][1,4]oxazin-3(4H)-one

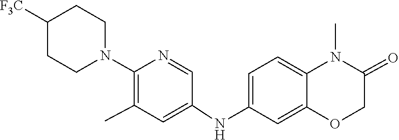 [SMILES: Cc1cc(Nc2ccc3c(c2)OCC(=O)N3C)cnc1N1CCC(C(F)(F)F)CC1]

To a solution of 5-methyl-6-(4-(trifluoromethyl)piperidin-1-yl) pyridin-3-amine (100 mg, 0.39 mmol), 7-bromo-4-methyl-2H-benzo[b][1,4]oxazin-3(4H)-one (112 mg, 0.46 mmol) in toluene (2 mL) was added tBuONa (74 mg, 0.77 mmol) and BrettPhos-Pd-G3 (35 mg, 0.038 mmol). The mixture was heated to 100° C. and stirred under nitrogen overnight. The resulting mixture was concentrated and the residue was purified by prep-HPLC to give compound 425 as a light yellow solid (14 mg, 8.22%). MS (ESI) m/z 421.0 [M+H]⁺. ¹H NMR (400 MHz, CDCl₃) δ 7.98 (d, J=2.4 Hz, 1H), 7.29 (s, 1H), 6.84 (d, J=8.4 Hz, 1H), 6.64 (d, J=2.4 Hz, 1H), 6.62 (s, 1H), 4.59 (s, 2H), 3.45-3.39 (m, 1H), 3.34 (s, 3H), 2.80 (t, J=12.0 Hz, 2H), 2.26 (s, 2H), 2.23-2.16 (m, 1H), 1.99-1.96 (m, 4H), 1.80-1.70 (m, 2H).

### Compound 143

### 7-((6-(2,6-dimethylmorpholino)-5-methylpyridin-3-yl)amino)-4-methyl-2H-benzo[b][1,4]oxazin-3(4H)-one

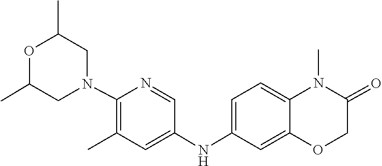 [SMILES: Cc1cc(Nc2ccc3c(c2)OCC(=O)N3C)cnc1N1CC(C)OC(C)C1]

To a solution of 6-(2,6-dimethylmorpholino)-5-methylpyridin-3-amine (90 mg, 0.41 mmol), 7-bromo-4-methyl-2H-benzo[b][1,4]oxazin-3(4H)-one (118 mg, 0.49 mmol) in toluene (2 mL) was added t-BuONa (78 mg, 0.81 mmol) and BrettPhos-Pd-G3 (36.7 mg, 0.041 mmol). The mixture was heated to 100° C. and stirred under nitrogen overnight. The resulting mixture was concentrated and the residue was purified by prep-HPLC to give 143 (14.6 mg, 8.80%) as a light yellow solid. MS (ESI) m/z 421.0 [M+H]⁺. ¹H NMR (400 MHz, CDCl₃) δ 7.98 (d, J=2.4 Hz, 1H), 7.25 (d, J=2.4 Hz, 1H), 6.83 (d, J=8.4 Hz, 1H), 6.64-6.58 (m, 2H), 4.58 (s, 2H), 3.88-3.80 (m, 2H), 3.33 (s, 3H), 3.17 (d, J=12.0 Hz, 2H), 2.60 (t, J=10.2 Hz, 2H), 2.27 (s, 3H), 1.23 (d, J=6.4 Hz, 6H).

### Compound 144

### 7-((4-(2,6-dimethylmorpholino)phenyl)amino)-2-methyl-2H-benzo[b][1,4]oxazin-3(4H)-one

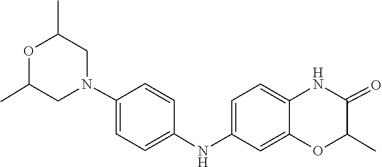 [SMILES: CC1CN(c2ccc(Nc3ccc4c(c3)OC(C)C(=O)N4)cc2)CC(C)O1]

To a solution of 4-(2,6-dimethylmorpholin-4-yl)aniline (100 mg, 0.485 mmol), 7-bromo-2-methyl-2,4-dihydro-1,4-benzoxazin-3-one (129 mg, 0.533 mmol), and t-BuONa (93.2 mg, 0.970 mmol) in toluene (2 mL) was added BrettPhos-Pd-G3 (44.0 mg, 0.0484 mmol). The resulting solution was stirred for 16 h at 100° C. under nitrogen atmosphere. The solution was diluted with water (20 mL) and EA (20 ml), then filtered through celite. The aqueous layer was extracted with EA (3×20 mL). The organic layers were combined, washed with brine, dried over anhydrous Na₂SO₄, filtered, and concentrated under reduced pressure. The residue was purified by pre-TLC (PE/EA=2/1) to provide compound 426 (28.2 mg, 15.43% yield) as a light yellow solid. MS (ESI) m/z 368.0 [M+H]⁺. ¹H NMR (400 MHz, DMSO-d₆) δ 10.38 (s, 1H), 7.68 (s, 1H), 6.98-6.83 (m, 4H), 6.70 (d, J=8.4 Hz, 1H), 6.57-6.45 (m, 2H), 4.56 (q, J=6.8 Hz, 1H), 3.73-3.64 (m, 2H), 3.43 (d, J=11.1 Hz, 2H), 2.18 (t, J=10.9 Hz, 2H), 1.38 (d, J=6.8 Hz, 3H), 1.14 (d, J=6.2 Hz, 6H).

### Compound 145

### 7-((4-(2,6-dimethylmorpholino)-3-methylphenyl)amino)-2H-benzo[b][1,4]oxazin-3(4H)-one

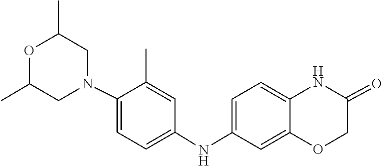 [SMILES: Cc1cc(Nc2ccc3c(c2)OCC(=O)N3)ccc1N1CC(C)OC(C)C1]

To a solution of 7-bromo-2H-benzo[b][1,4]oxazin-3(4H)-one (154 mg, 0.57 mmol) and 4-(2,6-dimethylmorpholino)-3-methylaniline (115 mg, 0.52 mmol) in 1,4-dioxane (2 mL) was added Pd₂(dba)₃ (48 mg, 0.05 mmol), Brettphos (56 mg, 0.10 mmol) and Cs₂CO₃ (341 mg, 1.04 mmol) under nitrogen atmosphere. The mixture was stirred at 100° C. for 12 h, the mixture was purified by prep TLC then purified by prep HPLC to give 7-((4-(2,6-dimethylmorpholino)-3-methylphenyl)amino)-2H-benzo[b][1,4]oxazin-3(4H)-one (54 mg, 28%) as an off-white solid. Mass (m/z): 368.4 [M+H]⁺. ¹H NMR (400 MHz, DMSO-d₆) δ 10.48 (s, 1H), 7.79 (s, 1H), 6.90 (d, J=8.3 Hz, 1H), 6.85-6.77 (m, 2H), 6.73 (d, J=8.3 Hz, 1H), 6.63-6.53 (m, 2H), 4.49 (s, 2H), 3.77-3.65 (m, 2H), 2.87-2.79 (m, 2H), 2.32-2.22 (m, 2H), 2.20 (s, 3H), 1.10 (d, J=6.2 Hz, 6H).

### Compound 146

### 7-((4-(8-oxa-3-azabicyclo[3.2.1]octan-3-yl)-3-methylphenyl)amino)-2H-benzo[b][1,4]oxazin-3(4H)-one

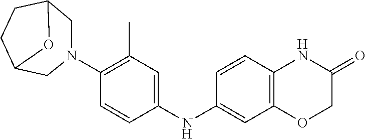

To a solution of 7-bromo-2H-benzo[b][1,4]oxazin-3(4H)-one (115 mg, 0.50 mmol) and 4-(8-oxa-3-azabicyclo[3.2.1]octan-3-yl)-3-methylaniline (100 mg, 0.46 mmol) in 1,4-dioxane (2 mL) was added Pd₂(dba)₃ (42 mg, 0.05 mmol), Brettphos (49 mg, 0.09 mmol) and Cs₂CO₃ (299 mg, 0.92 mmol) under nitrogen atmosphere. The mixture was stirred at 100° C. for 12 h, the mixture was purified by prep TLC then purified by prep HPLC to give 7-((4-(8-oxa-3-azabicyclo[3.2.1]octan-3-yl)-3-methylphenyl)amino)-2H-benzo[b][1,4]oxazin-3(4H)-one (23.5 mg, 14%) as a white solid. Mass (m/z): 366.4 [M+H]⁺. ¹H NMR (400 MHz, DMSO-d₆) δ 10.47 (s, 1H), 7.79 (s, 1H), 6.99-6.92 (m, 1H), 6.84-6.78 (m, 2H), 6.73 (d, J=8.4 Hz, 1H), 6.63-6.54 (m, 2H), 4.49 (s, 2H), 4.30 (dt, J=4.7, 2.2 Hz, 2H), 2.88-2.77 (m, 2H), 2.67-2.59 (m, 2H), 2.22 (s, 3H), 2.10-1.99 (m, 2H), 1.85-1.73 (m, 2H).

### Compound 147

### 7-((4-(2-ethylmorpholino)-2-methylphenyl)amino)-4-methyl-2H-benzo[b][1,4]oxazin-3(4H)-one

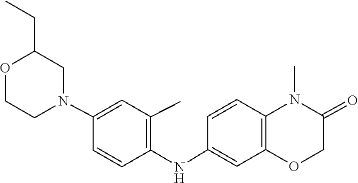 [SMILES: CCC1CN(c2ccc(Nc3ccc4c(c3)OCC(=O)N4C)c(C)c2)CCO1]

To a solution of 7-bromo-4-methyl-2H-benzo[b][1,4]oxazin-3(4H)-one (121 mg, 0.50 mmol) and 4-(2-ethylmorpholino)-2-methylaniline (100 mg, 0.45 mmol) in 1,4-dioxane (2 mL) was added Pd₂(dba)₃ (42 mg, 0.05 mmol), Xphos (43 mg, 0.09 mmol) and Cs₂CO₃ (296 mg, 0.91 mmol) under nitrogen atmosphere. The mixture was stirred at 100° C. for 12 h, the mixture was purified by prep TLC then purified by prep HPLC to give 7-((4-(2-ethylmorpholino)-2-methylphenyl)amino)-4-methyl-2H-benzo[b][1,4]oxazin-3(4H)-one (62 mg, 35.7%) as a grey solid. Mass (m/z): 382.4 [M+H]⁺. ¹H NMR (400 MHz, DMSO) δ 7.21 (s, 1H), 6.99 (d, J=8.6 Hz, 1H), 6.91 (d, J=8.7 Hz, 1H), 6.85 (d, J=2.8 Hz, 1H), 6.76 (dd, J=8.7, 2.9 Hz, 1H), 6.34 (dd, J=8.7, 2.5 Hz, 1H), 6.26 (d, J=2.5 Hz, 1H), 4.53 (s, 2H), 3.97-3.88 (m, 1H), 3.66-3.55 (m, 1H), 3.54-3.46 (m, 1H), 3.46-3.36 (m, 2H), 3.20 (s, 3H), 2.61 (td, J=11.8, 3.4 Hz, 1H), 2.36-2.26 (m, 1H), 2.12 (s, 3H), 1.55-1.42 (m, 2H), 0.95 (t, J=7.5 Hz, 3H).

### Compound 148

### 7-((4-(2-methylmorpholino)phenyl)amino)-2H-benzo[b][1,4]oxazin-3(4H)-one

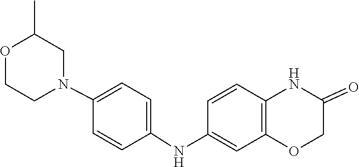 [SMILES: CC1CN(c2ccc(Nc3ccc4c(c3)OCC(=O)N4)cc2)CCO1]

To a solution of 7-bromo-2H-benzo[b][1,4]oxazin-3(4H)-one (129 mg, 0.57 mmol) and 4-(2-methylmorpholino)aniline (100 mg, 0.52 mmol) in 1,4-dioxane (2 mL) was added Pd₂(dba)₃ (47 mg, 0.05 mmol), Brettphos (56 mg, 0.10 mmol) and Cs₂CO₃ (339 mg, 1.04 mmol) under nitrogen atmosphere. The mixture was stirred at 100° C. for 12 h, the mixture was purified by prep TLC then purified by flash chromatography to give 7-((4-(2-methylmorpholino)phenyl)amino)-2H-benzo[b][1,4]oxazin-3(4H)-one (62 mg, 35%) as a light blue solid. Mass (m/z): 340.4 [M+H]⁺. ¹H NMR (400 MHz, DMSO-d₆) δ 10.44 (s, 1H), 7.69 (s, 1H), 6.98-6.90 (m, 2H), 6.90-6.82 (m, 2H), 6.71 (d, J=8.4 Hz, 1H), 6.56-6.47 (m, 2H), 4.48 (s, 2H), 3.93-3.84 (m, 1H), 3.69-3.57 (m, 2H), 3.48-3.39 (m, 1H), 3.38-3.30 (m, 1H), 2.62-2.51 (m, 1H), 2.25 (dd, J=11.7, 10.1 Hz, 1H), 1.14 (d, J=6.2 Hz, 3H).

### Compound 149

### 4-methyl-7-((4-(2-methylmorpholino)phenyl)amino)-2H-benzo[b][1,4]oxazin-3(4H)-one

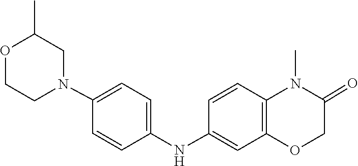 [SMILES: CC1CN(c2ccc(Nc3ccc4c(c3)OCC(=O)N4C)cc2)CCO1]

To a solution of 7-bromo-4-methyl-2H-benzo[b][1,4]oxazin-3(4H)-one (138 mg, 0.57 mmol) and 4-(2-methylmorpholino)aniline (100 mg, 0.52 mmol) in 1,4-dioxane (2 mL) was added Pd₂(dba)₃ (47 mg, 0.05 mmol), Xphos (49 mg, 0.10 mmol) and Cs₂CO₃ (339 mg, 1.04 mmol) under nitrogen atmosphere. The mixture was stirred at 100° C. for 12 h, the mixture was purified by prep TLC then purified by flash chromatography to give 4-methyl-7-((4-(2-methylmorpholino)phenyl)amino)-2H-benzo[b][1,4]oxazin-3(4H)-one (117 mg, 63%) as a light yellow solid. Mass (m/z): 354.4 [M+H]⁺. ¹H NMR (400 MHz, DMSO-d₆) δ 7.80 (s, 1H), 7.01-6.93 (m, 3H), 6.92-6.84 (m, 2H), 6.61 (dd, J=8.7, 2.5 Hz, 1H), 6.53 (d, J=2.4 Hz, 1H), 4.56 (s, 2H), 3.93-3.85 (m, 1H), 3.70-3.57 (m, 2H), 3.49-3.41 (m, 1H), 3.36 (d, J=11.1 Hz, 1H), 3.22 (s, 3H), 2.63-2.52 (m, 1H), 2.31-2.21 (m, 1H), 1.14 (d, J=6.2 Hz, 3H).

### Compound 150

### 7-((5-methyl-6-(4-(trifluoromethyl)piperidin-1-yl)pyridin-3-yl)amino)-2H-benzo[b][1,4]oxazin-3(4H)-one

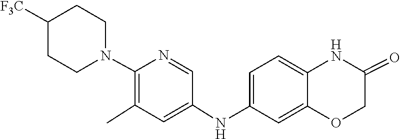 [SMILES: Cc1cc(Nc2ccc3c(c2)OCC(=O)N3)cnc1N1CCC(C(F)(F)F)CC1]

To a solution of 5-methyl-6-(4-(trifluoromethyl)piperidin-1-yl) pyridin-3-amine (100 mg, 0.39 mmol) and 7-bromo-2H-benzo[b][1,4]oxazin-3(4H)-one (105 mg, 0.46 mmol) in toluene (2 mL) was added t-BuONa (74 mg, 0.77 mmol) and BrettPhos-Pd-G3 (35 mg, 0.039 mmol). The mixture was heated to 100° C. and stirred under nitrogen overnight. The resulting mixture was concentrated and the residue was purified by prep-HPLC to give compound 150 as a light yellow solid (12.2 mg, 7.74%). MS (ESI) m/z 407.0 [M+H]⁺. ¹H NMR (400 MHz, DMSO-d₆) δ 10.59 (s, 1H), 8.26 (s, 1H), 7.76 (s, 1H), 7.53 (s, 1H), 6.84-6.61 (m, 3H), 4.53 (s, 2H), 3.90-3.70 (m, 1H), 3.50-3.35 (m, 2H), 2.90-2.82 (m, 2H), 2.27 (s, 3H), 1.97-1.86 (m, 2H), 1.70-1.55 (m, 2H)

### Compound 151

### 7-((6-(2,6-dimethylmorpholino)-5-methylpyridin-3-yl)amino)-2H-benzo[b][1,4]oxazin-3(4H)-one

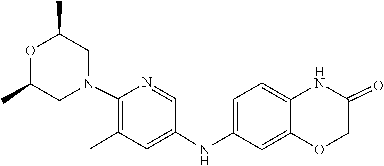 [SMILES: Cc1cc(Nc2ccc3c(c2)OCC(=O)N3)cnc1N1C[C@@H](C)O[C@@H](C)C1]

To a solution of cis-6-(2,6-dimethylmorpholino)-5-methylpyridin-3-amine (90 mg, 0.41 mmol) and 7-bromo-2H-benzo[b][1,4]oxazin-3(4H)-one (112 mg, 0.49 mmol) in toluene (2 mL) was added tBuONa (78 mg, 0.81 mmol) and BrettPhos-Pd-G3 (37 mg, 0.041 mmol). The mixture was heated to 100° C. and stirred under nitrogen overnight. The resulting mixture was concentrated and the residue was purified by prep-HPLC to give 151 as a light yellow solid (8.6 mg, 5.54%). ¹H NMR (400 MHz, DMSO-d⁶) δ 10.50 (s, 1H), 7.88 (s, 1H), 7.86 (d, J=2.8 Hz, 1H), 7.23 (d, J=2.8 Hz, 1H), 6.74 (d, J=8.4 Hz, 1H), 6.61-6.52 (m, 2H), 4.49 (s, 2H), 3.76-3.66 (m, 2H), 3.08 (d, J=11.6 Hz, 2H), 2.40-2.34 (m, 2H), 2.20 (s, 3H), 1.09 (d, J=2.4 Hz, 6H). MS (ESI) m/z 369.0 [M+H]⁺.

### Compound 152

### 7-((6-(2,6-dimethylmorpholino)-5-methylpyridin-3-yl)amino)-2H-benzo[b][1,4]oxazin-3(4H)-one

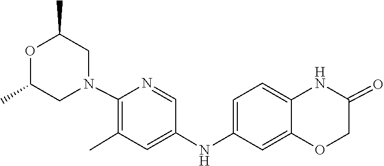 [SMILES: Cc1cc(Nc2ccc3c(c2)OCC(=O)N3)cnc1N1C[C@H](C)O[C@@H](C)C1]

To a solution of trans-6-(2,6-dimethylmorpholino)-5-methylpyridin-3-amine (40 mg, 0.18 mmol) and 7-bromo-2H-benzo[b][1,4]oxazin-3(4H)-one (49 mg, 0.22 mmol) in toluene (2 mL) was added tBuONa (35 mg, 0.36 mmol) and BrettPhos-Pd-G3 (16 mg, 0.018 mmol). The mixture was heated to 100° C. and stirred under nitrogen overnight. The resulting mixture was concentrated and the residue was purified by prep-HPLC to give compound 152 as a light yellow solid (5.0 mg, 3.32%). ¹H NMR (400 MHz, DMSO-d₆) δ 10.50 (s, 1H), 7.88 (s, 1H), 7.86 (d, J=2.8 Hz, 1H), 7.23 (d, J=2.8 Hz, 1H), 6.74 (d, J=8.4 Hz, 1H), 6.61-6.52 (m, 2H), 4.49 (s, 2H), 3.76-3.66 (m, 2H), 3.08 (d, J=11.6 Hz, 2H), 2.40-2.34 (m, 2H), 2.20 (s, 3H), 1.09 (d, J=2.4 Hz, 6H). MS (ESI) m/z 369.0 [M+H]⁺.

### Compound 153

### 7-((5-methyl-6-(4-(trifluoromethyl)piperidin-1-yl)pyridin-3-yl)amino)-2H-benzo[b][1,4]oxazin-3(4H)-one

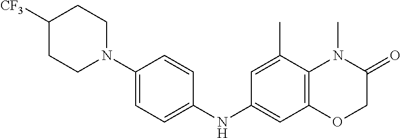 [SMILES: Cc1cc(Nc2ccc(N3CCC(C(F)(F)F)CC3)cc2)cc2c1N(C)C(=O)CO2]

A solution of 7-bromo-4,5-dimethyl-2H-1,4-benzoxazin-3-one (100 mg, 0.35 mmol), 4-[4-(trifluoromethyl)piperidin-1-yl]aniline (86 mg, 0.35 mmol), t-BuONa (75 mg, 0.78 mmol) and BrettPhos-Pd-G3 (35.4 mg, 0.039 mmol) in toluene (2 mL) was heated to 100° C. and stirred under nitrogen for 18 hrs. The resulting mixture was concentrated under reduced pressure and the residue was purified by prep-HPLC (0.05% NH₃) to give compound 153 as a light yellow solid (9.0 mg, 10.93% yield). ¹H NMR (400 MHz, DMSO-d⁶) δ 8.04 (s, 1H), 7.13-7.08 (m, 3H), 7.14-7.06 (m, 3H), 6.96 (d, J=8.4 Hz, 2H), 6.71 (dd, J=8.4, 2.0 Hz, 1H), 6.63 (d, J=2.4 Hz, 1H), 4.88 (t, J=5.6 Hz, 1H), 4.56 (s, 2H), 3.90 (t, J=6.0 Hz, 2H), 3.59-3.49 (m, 4H), 2.74-2.67 (m, 1H), 1.74-1.66 (m, 2H), 1.23-1.17 (m, 2H), 1.12 (d, J=6.4 Hz, 6H). MS (ESI) m/z 397.0 [M+H]⁺.

### Compound 154

### 7-((4-(2,6-dimethylmorpholino)phenyl)amino)-2-ethyl-2H-benzo[b][1,4]oxazin-3(4H)-one

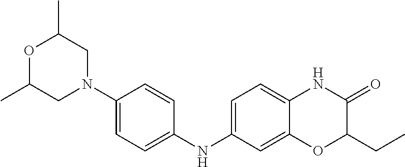 [SMILES: CCC1Oc2cc(Nc3ccc(N4CC(C)OC(C)C4)cc3)ccc2NC1=O]

To a solution of 4-(2,6-dimethylmorpholin-4-yl)aniline (100 mg, 0.485 mmol), 7-bromo-2-methyl-2,4-dihydro-1,4-benzoxazin-3-one (137 mg, 0.533 mmol), and t-BuONa (93.2 mg, 0.970 mmol) in toluene (2 mL) was added BrettPhos-Pd-G3 (44.0 mg, 0.0484 mmol). The resulting solution was stirred for 16 hrs at 100° C. under nitrogen atmosphere. The solution was diluted with water (20 mL) and EA (20 mL), then filtered through celite. The aqueous layer was extracted with EA (3×20 mL). The organic layers were combined, washed with brine, dried over anhydrous Na₂SO₄, filtered, and concentrated under reduced pressure. The residue was purified by pre-TLC (PE/EA=2/1) to provide compound 436 (31.1 mg, 15.64% yield) as a light pink solid. MS (ESI) m/z 382.0 [M+H]⁺. ¹H NMR (400 MHz, DMSO-d₆) δ 10.39 (s, 1H), 7.68 (s, 1H), 6.94 (d, J=9.0 Hz, 2H), 6.87 (d, J=9.0 Hz, 2H), 6.69 (d, J=8.5 Hz, 1H), 6.54-6.48 (m, 2H), 4.40 (dd, J=7.9, 4.5 Hz, 1H), 3.72-3.65 (m, 2H), 3.43 (d, J=10.5 Hz, 2H), 2.18 (t, J=10.2 Hz, 2H), 1.80-1.68 (m, 2H), 1.14 (d, J=6.2 Hz, 6H), 0.96 (t, J=7.4 Hz, 3H).

### Compound 155

### 7-((3,5-dimethyl-4-(4-(trifluoromethyl)piperidin-1-yl)phenyl)amino)-4-methyl-2H-benzo[b][1,4]oxazin-3(4H)-one

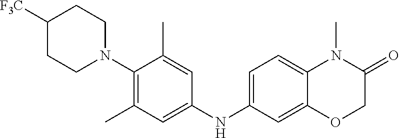 [SMILES: Cc1cc(Nc2ccc3c(c2)OCC(=O)N3C)cc(C)c1N1CCC(C(F)(F)F)CC1]

To a solution of 3,5-dimethyl-4-[4-(trifluoromethyl)piperidin-1-yl]aniline (100 mg, 0.367 mmol), 7-bromo-4-methyl-2H-1,4-benzoxazin-3-one (97.8 mg, 0.404 mmol), and t-BuONa (70.58 mg, 0.734 mmol) in toluene (2 mL) was added BrettPhos-Pd-G3 (33.3 mg, 0.0367 mmol). The resulting solution was stirred for 16 h at 100° C. under nitrogen atmosphere. The solution was diluted with water (20 mL) and EA (20 mL), then filtered through celite. The aqueous layer was extracted with EA (3×20 mL). The organic layers were combined, washed with brine, dried over anhydrous Na₂SO₄, filtered, and concentrated under reduced pressure. The residue was purified by prep-HPLC with CH₃CN/H₂O (0.05% NH₃) to provide compound 155 (8.7 mg, 5.42% yield) as a light pink solid. MS (ESI) m/z 434.0 [M+H]⁺. ¹H NMR (400 MHz, DMSO-d₆) δ 7.89 (s, 1H), 7.00 (d, J=8.7 Hz, 1H), 6.72 (dd, J=8.8, 2.4 Hz, 2H), 6.61 (dd, J=8.8, 2.4 Hz, 2H), 4.58 (s, 2H), 3.23 (s, 3H), 3.15 (t, J=10.8 Hz, 2H), 2.94-2.86 (m, 2H), 2.41-2.32 (m, 1H), 2.19 (d, J=10.5 Hz, 6H), 1.84-1.77 (m, 2H), 1.60-1.49 (m, 2H).

### Compound 156

### 6-fluoro-4-methyl-7-((4-(4-(trifluoromethyl)piperidin-1-yl)phenyl)amino)-2H-benzo[b][1,4]oxazin-3(4H)-one

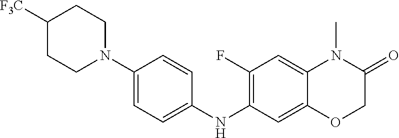 [SMILES: CN1C(=O)COc2cc(Nc3ccc(N4CCC(C(F)(F)F)CC4)cc3)c(F)cc21]

The mixture of 7-bromo-6-fluoro-4-methyl-2H-1,4-benzoxazin-3-one (100 mg, 0.384 mmol), 4-[4-(trifluoromethyl)piperidin-1-yl]aniline (85 mg, 0.346 mmol), t-BuONa (74 mg, 0.769 mmol) and BrettPhos Pd G3 (35.4 mg, 0.039 mmol) in toluene (2 mL) was heated to 100° C. and stirred for 18 h under nitrogen atmosphere. The resulting mixture was concentrated under reduced pressure and the residue was purified by column chromatography (EA:PE=1:1) to give compound 156 (45.5 mg, 26.84% yield) as a white solid. Mass (m/z): 424 [M+H]⁺. ¹H NMR (400 MHz, DMSO-d₆) δ 7.55 (s, 1H), 7.12 (d, J=12.4 Hz, 1H), 6.94-6.88 (m, 4H), 6.67 (d, J=8.0 Hz, 1H), 4.56 (s, 2H), 3.62 (d, J=12.4 Hz, 2H), 3.23 (s, 3H), 2.65-2.62 (m, 2H), 2.45-2.39 (m, 1H), 1.92-1.83 (m, 2H), 1.63-1.50 (m, 2H).

### Compound 157

### 4,6-dimethyl-7-((4-(4-(trifluoromethyl)piperidin-1-yl)cyclohexa-1,5-dien-1-yl)amino)-2H-benzo[b][1,4]oxazin-3(4H)-one

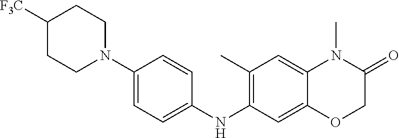 [SMILES: Cc1cc2c(cc1Nc1ccc(N3CCC(C(F)(F)F)CC3)cc1)OCC(=O)N2C]

The mixture of 7-bromo-4,6-dimethyl-2H-1,4-benzoxazin-3-one (100 mg, 0.35 mmol), 4-[4-(trifluoromethyl)piperidin-1-yl]aniline (86 mg, 0.35 mmol), t-BuONa (75 mg, 0.78 mmol) and BrettPhos-Pd-G3 (35.4 mg, 0.039 mmol) in toluene (2 mL) was heated to 100° C. and stirred under nitrogen for 18 hrs. The resulting mixture was concentrated under reduced pressure and the residue was purified by column chromatography (EA:PE=1:1) to give compound 157 (30 mg, 16.85% yield) as a white solid. Mass (m/z): 420 [M+H]⁺. ¹H NMR (400 MHz, DMSO-d₆) δ 6.93 (d, J=10.8 Hz, 2H), 6.86-6.81 (m, 4H), 6.52 (s, 1H), 4.49 (s, 2H), 3.57 (d, J=12 Hz, 2H), 3.17 (s, 3H), 2.65-2.52 (m, 2H), 2.41-2.35 (m, 1H), 2.13 (s, 3H), 1.84 (d, J=12 Hz, 2H), 1.58-1.45 (m, 2H).

### Compound 158

### 6-((4-(4-(trifluoromethyl)piperidin-1-yl)phenyl)amino)-3,4-dihydroquinolin-2(1H)-one

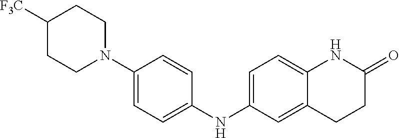 [SMILES: O=C1CCc2cc(Nc3ccc(N4CCC(C(F)(F)F)CC4)cc3)ccc2N1]

The mixture of compound 4-(4-(trifluoromethyl)piperidin-1-yl)aniline (200 mg, 0.82 mmol), 6-bromo-3,4-dihydroquinolin-2(1H)-one (200 mg, 0.88 mmol), t-BuONa (250 mg, 2.60 mmol), and Brettphos-Pd-G3 (75 mg, 0.083 mmol) in toluene (10 mL) was heated to 100° C. and stirred for six hours at N₂ atmosphere. TLC showed the reaction was completed. The resulting mixture was concentrated and the residue was purified through pre-TLC to give compound 158 (60 mg, 18.8% yield) as a white solid. MS (ESI) m/z 389.9 [M+H]⁺. ¹H NMR (400 MHz, CD₃OD) δ 9.85 (s, 1H), 7.58 (s, 1H), 7.00-6.65 (m, 7H), 3.57 (d, J=10.4 Hz, 2H), 3.16 (d, J=5.2 Hz, 1H), 2.82-2.72 (m, 2H), 2.70-2.54 (m, 2H), 2.42-2.35 (m, 2H), 1.92-1.82 (m, 2H), 1.65-1.50 (m, 2H).

### Compound 159

### 7-((4-(2,6-dimethylmorpholino)-3,5-difluorophenyl)amino)-2H-benzo[b][1,4]oxazin-3(4H)-one

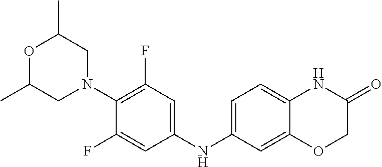 [SMILES: CC1CN(c2c(F)cc(Nc3ccc4c(c3)OCC(=O)N4)cc2F)CC(C)O1]

The titled compound 159 (60 mg, 29.3%) as a white solid was prepared according to the procedure outlined for compound 1. LC-MS (m/z) 390.2 [M+H]⁺. ¹H NMR (400 MHz, DMSO-d₆) δ 10.61 (s, 1H), 8.31 (s, 1H), 6.82 (d, J=8.3 Hz, 1H), 6.71 (dd, J=8.4, 2.3 Hz, 1H), 6.66 (d, J=2.3 Hz, 1H), 6.58-6.46 (m, 2H), 4.54 (s, 2H), 3.65 (dtt, J=8.7, 6.3, 3.7 Hz, 2H), 2.89-2.81 (m, 2H), 2.72-2.62 (m, 2H), 1.06 (d, J=6.3 Hz, 6H).

### Compound 160

### 7-((3,5-difluoro-4-(4-hydroxy-4-(trifluoromethyl)piperidin-1-yl)phenyl) amino)-4-methyl-2H-benzo[b][1,4]oxazin-3(4H)-one

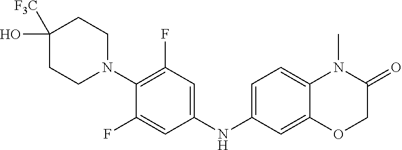 [SMILES: CN1C(=O)COc2cc(Nc3cc(F)c(N4CCC(O)(C(F)(F)F)CC4)c(F)c3)ccc21]

The titled compound 160 (16.3 mg, 36%) as a white solid was prepared according to the procedure outlined for compound 1. LC-MS (m/z) 458.2 [M+H]⁺. ¹H NMR (400 MHz, DMSO-d₆) δ 8.41 (s, 1H), 7.08 (d, J=8.7 Hz, 1H), 6.81 (dd, J=8.7, 2.5 Hz, 1H), 6.71 (d, J=2.4 Hz, 1H), 6.62-6.51 (m, 2H), 5.91 (s, 1H), 4.63 (s, 2H), 3.29 (d, J=12.8 Hz, 2H), 3.25 (s, 3H), 2.90-2.82 (m, 2H), 1.73 (dt, J=22.4, 8.5 Hz, 4H).

### Compound 161

### 7-((3,5-difluoro-4-(4-hydroxy-4-(trifluoromethyl)piperidin-1-yl)phenyl) amino)-4-methyl-2H-benzo[b][1,4]oxazin-3(4H)-one

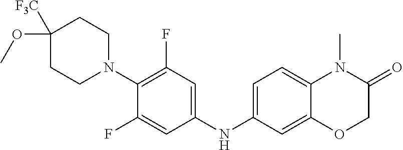 [SMILES: COC1(C(F)(F)F)CCN(c2c(F)cc(Nc3ccc4c(c3)OCC(=O)N4C)cc2F)CC1]

The titled compound 161 (80.6 mg, 30.2%) as a white solid was prepared according to the procedure outlined for compound 1. LC-MS (m/z) 472.2 [M+H]⁺. ¹H NMR (400 MHz, DMSO-d₆) δ 8.41 (s, 1H), 7.08 (d, J=8.7 Hz, 1H), 6.81 (dd, J=8.7, 2.4 Hz, 1H), 6.71 (d, J=2.4 Hz, 1H), 6.63-6.51 (m, 2H), 4.63 (s, 2H), 3.43 (s, 3H), 3.25 (s, 3H), 3.16 (t, J=11.7 Hz, 2H), 2.95-2.87 (m, 2H), 1.97 (dt, J=14.0, 2.4 Hz, 2H), 1.79 (td, J=13.0, 4.7 Hz, 2H).

### Compound 162

### 7-((4-(4-methoxy-4-(trifluoromethyl)piperidin-1-yl)phenyl)amino)-2H-benzo[b][1,4]oxazin-3(4H)-one

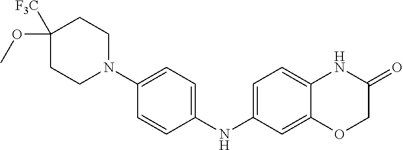 [SMILES: COC1(C(F)(F)F)CCN(c2ccc(Nc3ccc4c(c3)OCC(=O)N4)cc2)CC1]

The titled compound 162 (6 mg, 2.2%) as a saddle brown solid was prepared according to the procedure outlined for compound 1. LC-MS (m/z) 422.2 [M+H]⁺. ¹H NMR (400 MHz, DMSO-d₆) δ 10.45 (s, 1H), 7.71 (s, 1H), 6.97-6.87 (m, 4H), 6.71 (d, J=8.4 Hz, 1H), 6.58-6.48 (m, 2H), 4.48 (s, 2H), 3.45 (d, J=12.3 Hz, 2H), 3.41-3.36 (m, 3H), 2.74 (td, J=12.4, 2.4 Hz, 2H), 1.99 (dd, J=14.2, 2.6 Hz, 2H), 1.83 (td, J=13.1, 4.5 Hz, 2H).

### Compound 163

### 2-(4-methylpiperazin-1-yl)-1-(5-((4-(piperidin-1-yl)phenyl)amino) isoindolin-2-yl)ethan-1-one

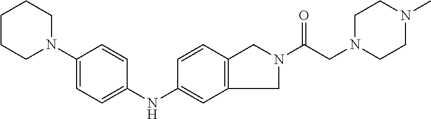 [SMILES: CN1CCN(CC(=O)N2Cc3ccc(Nc4ccc(N5CCCCC5)cc4)cc3C2)CC1]

The title compound 163 (16.8 mg) was prepared in a total yield of 78.0% as an orange solid from 4-(piperidin-1-yl)aniline (11.4 mg, 0.065 mmol), 1-(5-bromoisoindolin-2-yl)-2-(4-methylpiperazin-1-yl)ethan-1-one (16.9 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.08-6.96 (m, 3H), 6.94-6.83 (m, 4H), 4.70 (s, 2H), 4.56 (s, 2H), 3.24 (s, 2H), 3.08-2.86 (m, 4H), 2.76-2.54 (m, 8H), 2.34 (s, 3H), 1.73-1.64 (m, 4H), 1.57-1.48 (m, 2H). Mass (m/z): 434.3 [M+H]⁺.

### Compound 164

### 1-(5-((3-fluoro-4-(4-(trifluoromethyl)piperidin-1-yl)phenyl)amino) isoindolin-2-yl)-2-(4-methylpiperazin-]-yl)ethan-]-one

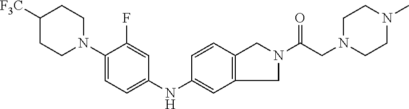 [SMILES: CN1CCN(CC(=O)N2Cc3ccc(Nc4ccc(N5CCC(C(F)(F)F)CC5)c(F)c4)cc3C2)CC1]

The title compound 164 (17.2 mg) was prepared in a total yield of 66.3% as an orange solid from 3-fluoro-4-(4-(trifluoromethyl)piperidin-1-yl) aniline (17.1 mg, 0.065 mmol), 1-(5-bromoisoindolin-2-yl)-2-(4-methylpiperazin-1-yl)ethan-1-one (16.9 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.18-7.10 (m, 1H), 7.00-6.91 (m, 3H), 6.84-6.75 (m, 2H), 4.81 (s, 2H), 4.65 (s, 2H), 3.39-3.32 (m, 4H), 2.86-2.58 (m, 10H), 2.25 (m, 1H), 1.98-1.89 (m, 2H), 1.79-1.66 (m, 2H). Mass (m/z): 520.3 [M+H]⁺.

### Compound 165

### N-(4-(4,4-difluoropiperidin-1-yl)phenyl)isoindolin-5-amine

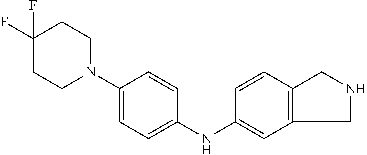 [SMILES: FC1(F)CCN(c2ccc(Nc3ccc4c(c3)CNC4)cc2)CC1]

The title compound 165 (11.8 mg) was prepared in a total yield of 71.7% as a yellow solid from 4-(4,4-difluoropiperidin-1-yl)aniline (13.8 mg, 0.065 mmol), 5-bromoisoindoline (9.9 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.17-6.89 (m, 7H), 4.48 (s, 2H), 4.43 (s, 2H), 3.27-3.21 (m, 4H), 2.14-2.01 (m, 4H). Mass (m/z): 330.2 [M+H]⁺.

### Compound 166

### 1-(5-((4-(4,4-difluoropiperidin-1-yl)phenyl)amino)isoindolin-2-yl) ethan-1-one

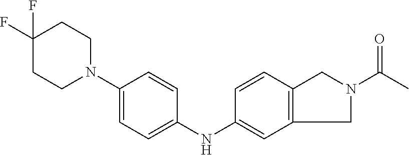 [SMILES: CC(=O)N1Cc2ccc(Nc3ccc(N4CCC(F)(F)CC4)cc3)cc2C1]

The title compound 166 (15.2 mg) was prepared in a total yield of 81.9% as a yellow solid from 4-(4,4-difluoropiperidin-1-yl)aniline (13.8 mg, 0.065 mmol), 1-(5-bromoisoindolin-2-yl)ethan-1-one (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.15-6.78 (m, 7H), 4.75 (s, 2H), 4.61 (s, 2H), 3.26-3.18 (m, 4H), 2.15 (s, 3H), 2.12-1.97 (m, 4H). Mass (m/z): 372.2 [M+H]⁺.

### Compound 167

### N-(4-(4,4-difluoropiperidin-1-yl)phenyl)-2-ethylisoindolin-5-amine

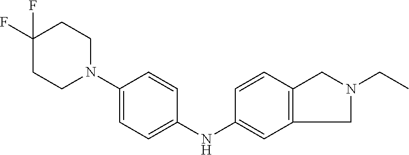 [SMILES: CCN1Cc2ccc(Nc3ccc(N4CCC(F)(F)CC4)cc3)cc2C1]

The title compound 167 (12.8 mg) was prepared in a total yield of 71.7% as a yellow solid from 4-(4,4-difluoropiperidin-1-yl)aniline (13.8 mg, 0.065 mmol), 5-bromo-2-ethylisoindoline (11.3 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.22-6.61 (m, 7H), 4.68 (s, 2H), 4.55 (s, 2H), 3.47 (q, J=7.2 Hz, 2H), 3.27-3.15 (m, 4H), 2.37-2.16 (m, 4H), 1.42 (t, J=7.2 Hz, 3H). Mass (m/z): 358.2 [M+H]⁺.

### Compound 168

### 6-((4-(4,4-difluoropiperidin-1-yl)phenyl)amino)-2-methylisoindolin-1-one

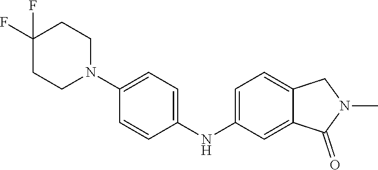 [SMILES: CN1Cc2ccc(Nc3ccc(N4CCC(F)(F)CC4)cc3)cc2C1=O]

The title compound 168 (3.3 mg) was prepared in a total yield of 3.9% as a pale yellow solid from 4-(4,4-difluoropiperidin-1-yl)aniline (50 mg, 0.23 mmol), 6-bromo-2-methylisoindolin-1-one (53 mg, 0.23 mmol), (9,9-dimethyl-9H-xanthene-4,5-diyl)bis(diphenylphosphane) (10.91 mg, 0.019 mmol, 0.08 equivs), [1,1′-Bis(diphenylphosphino) ferrocene]dichloropalladium (II) (6.9 mg, 0.009 mmol, 0.04 equivs) and cesium carbonate (153.5 mg, 0.47 mmol, 2.0 equivs) according to the procedure for compound 1. ¹H NMR (400 MHz, Methanol-d₄) δ 7.36 (dd, J=5.2, 2.9 Hz, 1H), 7.32-7.28 (m, 1H), 7.17-7.13 (m, 1H), 7.08 (d, J=8.7 Hz, 2H), 7.03-6.97 (m, 2H), 4.38 (s, 2H), 3.25 (s 3H), 3.17 (d, J=3.0 Hz, 4H), 2.16-2.00 (m, 4H). ¹⁹F NMR (376 MHz, Methanol-d₄) δ −99.32. LC-MS (m/z) 358.6 [M+H]⁺.

### Compound 169

### 6-((3-fluoro-4-(4-(trifluoromethyl)piperidin-1-yl)phenyl)amino)-2-methylisoindolin-1-one

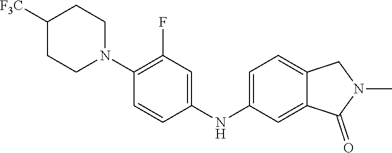 [SMILES: CN1Cc2ccc(Nc3ccc(N4CCC(C(F)(F)F)CC4)c(F)c3)cc2C1=O]

The title compound 169 (4.5 mg) was prepared in a total yield of 5.0% as a pale yellow solid from 3-fluoro-4-(4-(trifluoromethyl)piperidin-1-yl) aniline (50 mg, 0.24 mmol, 1.0 equivs), 6-bromo-2-methylisoindolin-1-one (53 mg, 0.24 mmol, 1.0 equivs), (9,9-dimethyl-9H-xanthene-4,5-diyl) bis(diphenylphosphane) (10.91 mg, 0.019 mmol, 0.08 equivs), dichloro[1,1′-bis(diphenylphosphino)ferrocene]palladium(II) (6.9 mg, 0.009 mmol, 0.04 equivs) and cesium carbonate (92 mg, 0.28 mmol, 1.2 equivs) according to the procedure for compound 1. ¹H NMR (400 MHz, Methanol-d₄) δ 7.74 (dt, J=7.5, 1.0 Hz, 1H), 7.71 (dd, J=5.7, 3.3 Hz, 1H), 7.64-7.58 (m, 2H), 7.55 (ddd, J=7.6, 5.7, 1.0 Hz, 2H), 7.48 (ddd, J=8.3, 7.1, 1.5 Hz, 1H), 4.48 (s, 2H), 3.22 (d, J=2.3 Hz, 1H), 3.19 (s, 3H), 3.17 (d, J=5.1 Hz, 1H), 1.78-1.65 (m, 2H), 1.50-1.40 (m, 2H), 1.01-0.94 (m, 3H). ¹⁹F NMR (376 MHz, Methanol-d₄) δ −75.43 (d, J=8.3 Hz), −76.95, −123.72, −123.75. Mass (m/z): 408.3 [M+H]⁺.

### Compound 170

### 2-methyl-6-((4-(4-methylpiperidin-1-yl)phenyl)amino)isoindolin-1-one

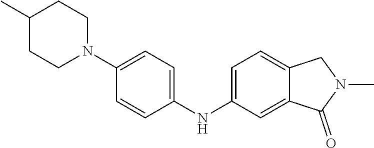

The title compound 170 (21.4 mg) was prepared in a yield of 24.38% as a pale yellow solid from 6-bromo-2-methylisoindolin-1-one (50 mg, 0.22 mmol) and 4-(4-methylpiperidin-1-yl)aniline (42 mg, 0.22 mmol), according to the procedure for compound 1. ¹H NMR (400 MHz, Methanol-d₄) δ 7.75-7.46 (m, 2H), 7.36 (d, J=8.9 Hz, 2H), 7.28-6.95 (m, 3H), 4.40 (s, 2H), 3.67-3.45 (m, 2H), 3.16 (s, 3H), 3.04-2.96 (m, 1H), 1.86 (d, J=11.7 Hz, 2H), 1.64 (q, J=13.2, 11.6 Hz, 2H), 1.56-1.42 (m, 2H), 1.03 (d, J=6.4 Hz, 3H). LC-MS (m/z) 336.2 [M+H]⁺.

### Compound 171

### 2-methyl-6-((4-(4-(trifluoromethyl)piperidin-1-yl)phenyl)amino) isoindolin-1-one

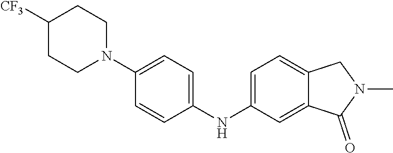 [SMILES: CN1Cc2ccc(Nc3ccc(N4CCC(C(F)(F)F)CC4)cc3)cc2C1=O]

The title compound 171 (12.2 mg) was prepared in a yield of 14.17% as a pale yellow solid from 6-bromo-2-methylisoindolin-1-one (50 mg, 0.22 mmol) and 4-(4-(trifluoromethyl)piperidin-1-yl)aniline (54 mg, 0.22 mmol), according to the procedure for compound 1. ¹H NMR (400 MHz, Methanol-d₄) δ 7.67-7.46 (m, 2H), 7.42-7.28 (m, 2H), 7.24-7.04 (m, 2H), 6.99 (d, J=7.9 Hz, 1H), 4.36 (d, J=7.9 Hz, 2H), 3.65 (d, J=12.3 Hz, 1H), 3.16 (s, 3H), 2.68 (t, J=11.8 Hz, 1H), 2.28 (s, 1H), 1.80-1.65 (m, 2H), 1.46 (h, J=7.3 Hz, 1H), 1.39-1.18 (m, 3H). LC-MS (m/z) 390.3 [M+H]⁺.

### Compound 172

### 5-((4-(4,4-difluoropiperidin-1-yl)phenyl)amino)-2-methylisoindolin-1-one

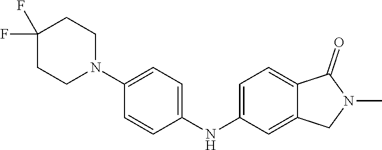 [SMILES: CN1Cc2cc(Nc3ccc(N4CCC(F)(F)CC4)cc3)ccc2C1=O]

The title compound 172 (93.8 mg) was prepared in a yield of 59.33% as a pale yellow powder from 5-bromo-2-methylisoindolin-1-one (100 mg, 0.44 mmol) and 4-(4,4-difluoropiperidin-1-yl)aniline (94 mg, 0.44 mmol), according to the procedure for compound 1. ¹H NMR (400 MHz, Methanol-d₄) δ 7.50 (d, J=8.5 Hz, 1H), 7.14-6.96 (m, 6H), 4.32 (s, 2H), 3.27 (s, 3H), 3.12 (s, 4H), 2.09 (m, 4H). LC-MS (m/z) 358.2 [M+H]⁺.

### Compound 173

### 2-methyl-5-((4-(4-(trifluoromethyl)piperidin-1-yl)phenyl)amino) isoindolin-1-one

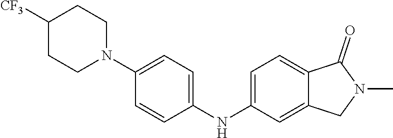 [SMILES: CN1Cc2cc(Nc3ccc(N4CCC(C(F)(F)F)CC4)cc3)ccc2C1=O]

To a solution of 4-(4-(trifluoromethyl)piperidin-1-yl)aniline (108 mg, 0.44 mmol, 1.0 equivs) and 5-bromo-2-methylisoindolin-1-one (100 mg, 0.44 mmol, 1.0 equivs) in 1,4-dioxane (5 mL) was added (9,9-dimethyl-9H-xanthene-4,5-diyl)bis(diphenylphosphane) (20.48 mg, 0.035 mmol, 0.08 equivs), [1,1′-Bis(diphenylphosphino)ferrocene]dichloropalladium (II) (12.95 mg, 0.018 mmol, 0.04 equivs) and cesium carbonate (216.18 mg, 0.66 mmol, 2.0 equivs) respectively under argon atmosphere. The resulting mixture was heated to 110° C. and stirred for overnight at the same temperature. The reaction was diluted with water (10 mL) and extracted with ethyl acetate (5 mL) 3 times. The organic layer was combined and washed with water, sat·NaHCO₃ (aq), and brine respectively. Then dried over MgSO₄, filtered, and concentrated under reduced pressure. The residue was purified by silica gel column chromatography (petroleum ether/AcOEt, 1/4) to give 58.4 mg of compound 1 in a yield of 33.90% as a blue powder. ¹H NMR (400 MHz, Methanol-d₄) δ 7.50 (d, J=8.4 Hz, 1H), 7.11 (d, J=8.5 Hz, 2H), 7.04-6.96 (m, 3H), 6.95-6.90 (m, 1H), 4.33 (s, 2H), 3.68 (d, J=12.4 Hz, 2H), 3.11 (s, 3H), 2.69 (s, 2H), 2.34-2.21 (m, 1H), 2.05-1.89 (m, 2H), 1.79-1.64 (m, 2H). LC-MS (m/z) 390.2 [M+H]⁺.

### Compound 174

### 2-methyl-5-((4-(4-methylpiperidin-1-yl)phenyl)amino)isoindolin-1-one

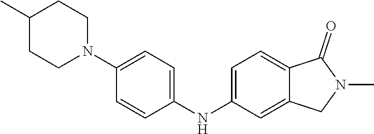

The title compound 174 (37.9 mg) was prepared in a yield of 25.54% as a pale yellow powder from 5-bromo-2-methylisoindolin-1-one (100 mg, 0.44 mmol) and 4-(4-methylpiperidin-1-yl)aniline (84 mg, 0.44 mmol), according to the procedure for compound 1. ¹H NMR (400 MHz, Methanol-d₄) δ 7.49 (d, J=8.4 Hz, 1H), 7.14-7.05 (m, 2H), 7.02-6.95 (m, 3H), 6.92 (dd, J=8.4, 2.1 Hz, 1H), 4.30 (s, 2H), 3.54 (d, J=11.8 Hz, 2H), 3.10 (s, 3H), 2.64 (t, J=12.1 Hz, 2H), 1.82-1.68 (m, 2H), 1.57-1.42 (m, 1H), 1.41-1.30 (m, 2H), 0.99 (d, J=6.3 Hz, 3H). LC-MS (m/z) 336.3 [M+H]⁺.

### Compound 175

### 2-ethyl-N-(4-(4-(trifluoromethyl)piperidin-1-yl)phenyl)isoindolin-5-amine

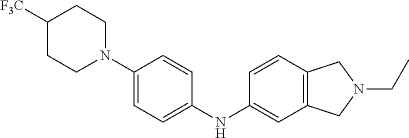 [SMILES: CCN1Cc2ccc(Nc3ccc(N4CCC(C(F)(F)F)CC4)cc3)cc2C1]

The title compound 175 (14.7 mg) was prepared in a total yield of 75.6% as a yellow solid from 4-(4-(trifluoromethyl)piperidin-1-yl)aniline (15.9 mg, 0.065 mmol), 5-bromo-2-ethylisoindoline (11.3 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.07 (d, J=8.0 Hz, 1H), 7.04-7.00 (m, 2H), 6.97-6.93 (m, 2H), 6.90-6.83 (m, 2H), 4.05 (s, 4H), 3.65-3.55 (m, 2H), 2.97 (q, J=7.2 Hz, 2H), 2.70-2.61 (m, 2H), 2.26 (m, 1H), 2.00-1.89 (m, 2H), 1.79-1.64 (m, 2H), 1.25 (t, J=7.2 Hz, 3H). Mass (m/z): 390.3 [M+H]⁺.

### Compound 176

### 2-ethyl-N-(4-(4-methylpiperidin-1-yl)phenyl)isoindolin-5-amine

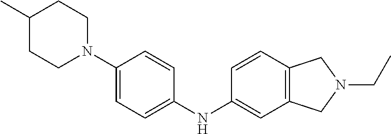 [SMILES: CCN1Cc2ccc(Nc3ccc(N4CCC(C)CC4)cc3)cc2C1]

The title compound 176 (13.9 mg) was prepared in a total yield of 82.9% as a yellow solid from 4-(4-methylpiperidin-1-yl)aniline (12.4 mg, 0.065 mmol), 5-bromo-2-ethylisoindoline (11.3 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.10-6.81 (m, 7H), 4.03 (s, 4H), 3.55-3.43 (m, 2H), 2.96 (q, J=7.2 Hz, 2H), 2.71-2.54 (m, 2H), 1.79-1.71 (m, 2H), 1.56-1.43 (m, 1H), 1.42-1.29 (m, 2H), 1.25 (t, J=7.2 Hz, 3H), 0.99 (d, J=6.4 Hz, 3H). Mass (m/z): 336.2 [M+H]⁺.

### Compound 177

### 2-ethyl-N-(4-(piperidin-1-yl)-3-(trifluoromethyl)phenyl)isoindolin-5-amine

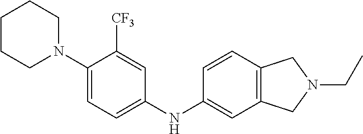 [SMILES: CCN1Cc2ccc(Nc3ccc(N4CCCCC4)c(C(F)(F)F)c3)cc2C1]

The title compound 177 (17.3 mg) was prepared in a total yield of 88.7% as a yellow solid from 4-(piperidin-1-yl)-3-(trifluoromethyl)aniline (15.9 mg, 0.065 mmol), 5-bromo-2-ethylisoindoline (11.3 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.37-7.23 (m, 3H), 7.19 (d, J=8.0 Hz, 1H), 7.07-6.98 (m, 2H), 4.38 (s, 2H), 4.36 (s, 2H), 3.25 (q, J=7.2 Hz, 2H), 2.85-2.70 (m, 4H), 1.71-1.62 (m, 4H), 1.58-1.50 (m, 2H), 1.35 (t, J=7.2 Hz, 3H). Mass (m/z): 390.3 [M+H]⁺.

### Compound 178

### 2-methyl-5-((4-(piperidin-1-yl)-3-(trifluoromethyl)phenyl)amino) isoindolin-1-one

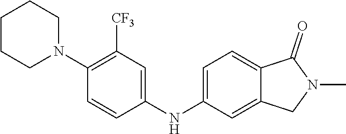 [SMILES: CN1Cc2cc(Nc3ccc(N4CCCCC4)c(C(F)(F)F)c3)ccc2C1=O]

The title compound 178 (58.4 mg) was prepared in a yield of 25.72% as a white powder from 5-bromo-2-methylisoindolin-1-one (100 mg, 0.44 mmol) and 4-(piperidin-1-yl)-3-(trifluoromethyl)aniline (108 mg, 0.44 mmol), according to the procedure for compound 1. ¹H NMR (400 MHz, Methanol-d₄) δ 7.58 (d, J=8.4 Hz, 1H), 7.44-7.39 (m, 2H), 7.37 (t, J=1.5 Hz, 1H), 7.16-7.12 (m, 1H), 7.06 (dd, J=8.4, 2.1 Hz, 1H), 4.40 (s, 2H), 3.14 (s, 3H), 2.87-2.78 (m, 4H), 1.69 (p, J=5.7 Hz, 4H), 1.58 (d, J=6.0 Hz, 2H). LC-MS (m/z) 387.9 [M−H]⁻.

### Compound 179

### 2-(cyclopropylmethyl)-5-((4-(4-(trifluoromethyl)piperidin-1-yl) phenyl)amino)isoindolin-1-one

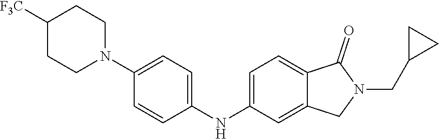 [SMILES: O=C1c2ccc(Nc3ccc(N4CCC(C(F)(F)F)CC4)cc3)cc2CN1CC1CC1]

The title compound 179 (17.2 mg) was prepared in a yield of 9.78% as a pale yellow powder from 5-bromo-2-(cyclopropylmethyl)isoindolin-1-one (100 mg, 0.41 mmol) and 4-(4-(trifluoromethyl)piperidin-1-yl)aniline (120 mg, 0.41 mmol), according to the procedure for compound 1. ¹H NMR (400 MHz, DMSO-d₆) δ 8.31 (s, 1H), 7.41 (d, J=8.3 Hz, 1H), 7.07 (d, J=8.9 Hz, 2H), 6.99 (d, J=2.1 Hz, 1H), 6.98-6.92 (m, 2H), 6.90 (dt, J=8.4, 2.0 Hz, 1H), 4.40 (s, 2H), 3.69 (d, J=12.3 Hz, 2H), 2.71-2.61 (m, 2H), 1.89 (d, J=13.6 Hz, 2H), 1.57 (qd, J=12.4, 4.0 Hz, 2H), 1.24 (s, 2H), 1.04-0.94 (m, 1H), 0.89-0.82 (d, J=7.2 Hz, 1H), 0.51-0.44 (m, 2H), 0.30-0.22 (m, 2H). LC-MS (m/z) 430.3 [M+H]⁺.

### Compound 180

### 2-ethyl-5-((4-(4-(trifluoromethyl)piperidin-1-yl)phenyl)amino) isoindolin-1-one

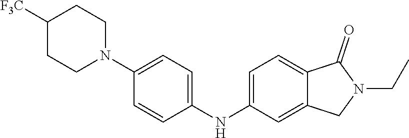 [SMILES: CCN1Cc2cc(Nc3ccc(N4CCC(C(F)(F)F)CC4)cc3)ccc2C1=O]

The title compound 180 (73.1 mg) was prepared in a yield of 44.26% as a pale yellow powder from 4-(4-(trifluoromethyl)piperidin-1-yl)aniline (100 mg, 0.41 mmol) and 5-bromo-2-ethylisoindolin-1-one (98 mg, 0.41 mmol) according to the procedure for compound 1. ¹H NMR (400 MHz, Chloroform-d) δ 7.58 (d, J=8.4 Hz, 1H), 7.49-7.35 (m, 1H), 7.15 (s, 2H), 6.98 (d, J=10.0 Hz, 4H), 4.32 (d, J=4.1 Hz, 2H), 3.69 (d, J=12.3 Hz, 2H), 3.61 (qd, J=7.3, 2.8 Hz, 2H), 3.42-3.33 (m, 1H), 2.71 (t, J=12.3 Hz, 2H), 2.19 (ddt, J=12.6, 8.3, 4.0 Hz, 1H), 2.00 (d, J=13.1 Hz, 2H), 1.87-1.70 (m, 2H), 1.25 (td, J=7.2, 3.1 Hz, 2H). Mass (m/z): 404.2 [M+H]⁺.

### Compound 181

### 2,2-dimethyl-1-(5-((4-(4-(trifluoromethyl)piperidin-1-yl)phenyl)amino) isoindolin-2-yl)propan-1-one

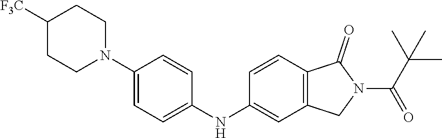 [SMILES: CC(C)(C)C(=O)N1Cc2cc(Nc3ccc(N4CCC(C(F)(F)F)CC4)cc3)ccc2C1=O]

The title compound 181 (17.0 mg) was prepared in a total yield of 76.4% as a yellow solid from 4-(4-(trifluoromethyl)piperidin-1-yl)aniline (15.9 mg, 0.065 mmol), 1-(5-bromoisoindolin-2-yl)-2,2-dimethylpropan-1-one (14.1 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.27-6.75 (m, 7H), 4.96 (s, 2H), 4.67 (s, 2H), 3.83-3.41 (m, 2H), 2.71-2.57 (m, 2H), 2.27 (m, 1H), 2.07-1.89 (m, 2H), 1.81-1.62 (m, 2H), 1.32 (s, 9H). Mass (m/z): 446.3 [M+H]⁺.

### Compound 182

### 5-((4-(4-(trifluoromethyl)piperidin-1-yl)phenyl)amino)isoindolin-1-one

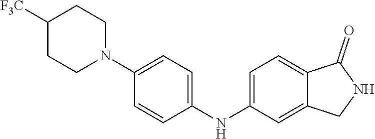 [SMILES: O=C1NCc2cc(Nc3ccc(N4CCC(C(F)(F)F)CC4)cc3)ccc21]

The title compound 182 (1.1 mg) was prepared in a yield of 1.4% as a pale yellow powder from 4-(4-(trifluoromethyl)piperidin-1-yl)aniline (50 mg, 0.20 mmol) and 5-bromoisoindolin-1-one (43 mg, 0.20 mmol) according to the procedure for compound 1. ¹H NMR (400 MHz, Methanol-d₄) δ 7.53 (d, J=8.4 Hz, 1H), 7.15-7.09 (m, 2H), 7.03-6.98 (m, 3H), 6.95 (dd, J=8.3, 2.1 Hz, 1H), 4.32 (s, 2H), 3.69 (d, J=12.4 Hz, 2H), 2.70 (td, J=12.5, 2.4 Hz, 2H), 1.98 (d, J=13.4 Hz, 2H), 1.72 (qd, J=12.4, 4.5 Hz, 3H). Mass (m/z): 376.2 [M+H]⁺.

### Compound 183

### 5-((5-fluoro-6-(piperidin-1-yl)pyridin-3-yl)amino)-2-methylisoindolin-1-one

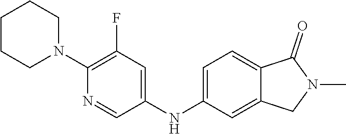 [SMILES: CN1Cc2cc(Nc3cnc(N4CCCCC4)c(F)c3)ccc2C1=O]

The title compound 183 (24.6 mg) was prepared in a yield of 28.22% as a pale yellow powder from 5-fluoro-6-(piperidin-1-yl)pyridin-3-amine (50 mg, 0.26 mmol) and 5-bromo-2-methylisoindolin-1-one (58 mg, 0.26 mmol) according to the procedure for compound 1. ¹H NMR (400 MHz, DMSO-d₆) δ 8.53 (s, 1H), 7.94 (dt, J=2.3, 1.2 Hz, 1H), 7.44 (d, J=8.3 Hz, 1H), 7.42-7.36 (m, 1H), 7.04 (d, J=2.2 Hz, 1H), 6.93 (dt, J=8.3, 1.9 Hz, 1H), 4.31 (s, 2H), 2.99 (s, 3H), 1.66-1.52 (m, 6H). Mass (m/z): 341.7 [M+H]⁺.

### Compound 184

### 2-(cyclopropylmethyl)-5-((4-(4-methylpiperidin-1-yl)phenyl)amino) isoindolin-1-one

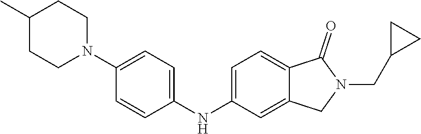 [SMILES: CC1CCN(c2ccc(Nc3ccc4c(c3)CN(CC3CC3)C4=O)cc2)CC1]

The title compound 184 (46.3 mg) was prepared in a yield of 46.92% as a pale yellow powder from 4-(4-methylpiperidin-1-yl)aniline (50 mg, 0.26 mmol) and 5-bromo-2-(cyclopropylmethyl)isoindolin-1-one (58 mg, 0.26 mmol) according to the procedure for compound 1. ¹H NMR (400 MHz, DMSO-d₆) δ 8.28 (s, 1H), 7.41 (d, J=8.3 Hz, 1H), 7.09-7.02 (m, 2H), 6.97 (d, J=2.1 Hz, 1H), 6.94 (s, 1H), 6.92-6.87 (m, 2H), 4.40 (s, 2H), 3.65-3.50 (m, 2H), 3.29 (d, J=7.1 Hz, 2H), 2.60 (t, J=11.9 Hz, 2H), 1.74-1.65 (m, 2H), 1.47 (dq, J=11.0, 6.6, 5.3 Hz, 1H), 1.26 (dd, J=12.7, 9.0 Hz, 3H), 0.95 (d, J=6.5 Hz, 3H), 0.53-0.45 (m, 2H), 0.31-0.22 (m, 2H). Mass (m/z): 376.3 [M+H]⁺.

### Compound 185

### 2-(cyclopropylmethyl)-5-((4-(piperidin-1-yl)phenyl)amino)isoindolin-1-one

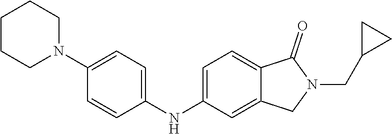 [SMILES: O=C1c2ccc(Nc3ccc(N4CCCCC4)cc3)cc2CN1CC1CC1]

The title compound 185 (72.1 mg) was prepared in a yield of 70.31% as a pale yellow powder from 4-(piperidin-1-yl)aniline (50 mg, 0.28 mmol) and 5-bromo-2-(cyclopropylmethyl)isoindolin-1-one (83 mg, 0.31 mmol) according to the procedure for compound 1. ¹H NMR (400 MHz, DMSO-d₆) δ 7.41 (d, J=8.3 Hz, 1H), 7.09-7.02 (m, 2H), 6.97 (t, J=2.3 Hz, 1H), 6.95-6.86 (m, 3H), 4.39 (s, 2H), 3.29 (d, J=7.1 Hz, 2H), 3.05 (d, J=10.9 Hz, 4H), 1.71-1.57 (m, 4H), 1.56-1.45 (m, 2H), 1.04-0.93 (m, 1H), 0.52-0.42 (m, 2H), 0.29-0.21 (m, 2H). Mass (m/z): 362.1 [M+H]⁺.

### Compound 186

### 1-(5-((4-(4-(trifluoromethyl)piperidin-1-yl)phenyl)amino)isoindolin-2-yl) ethan-1-one

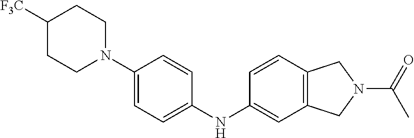 [SMILES: CC(=O)N1Cc2ccc(Nc3ccc(N4CCC(C(F)(F)F)CC4)cc3)cc2C1]

The title compound 186 (16.7 mg) was prepared in a total yield of 82.6% as a yellow solid from 4-(4-(trifluoromethyl)piperidin-1-yl)aniline (15.9 mg, 0.065 mmol), 1-(5-bromoisoindolin-2-yl)ethan-1-one (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.18-6.68 (m, 7H), 4.70 (s, 4H), 3.75-3.58 (m, 2H), 2.79-2.68 (m, 2H), 2.18 (m, 1H), 2.15 (s, 3H), 2.04-1.93 (m, 2H), 1.85-1.70 (m, 2H). Mass (m/z): 404.2 [M+H]⁺.

### Compound 187

### 2-methyl-6-((4-(4-(trifluoromethyl)piperidin-1-yl)phenyl)amino)benzo[d]isoxazol-3(2H)-one

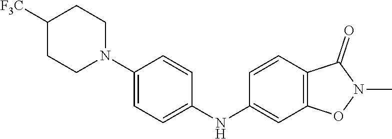 [SMILES: Cn1oc2cc(Nc3ccc(N4CCC(C(F)(F)F)CC4)cc3)ccc2c1=O]

The title compound 187 (16.4 mg) was prepared in a total yield of 83.9% as a yellow solid from 4-(4-(trifluoromethyl)piperidin-1-yl)aniline (15.9 mg, 0.065 mmol), 6-bromo-2-methylbenzo[d]isoxazol-3(2H)-one (11.4 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.56 (d, J=8.8 Hz, 1H), 7.13 (d, J=8.8 Hz, 2H), 6.95 (d, J=8.8 Hz, 2H), 6.66 (d, J=8.8 Hz, 1H), 6.55-6.49 (m, 1H), 3.77-3.69 (m, 2H), 3.54 (s, 3H), 2.77-2.67 (m, 2H), 2.17 (m, 1H), 2.05-1.95 (m, 2H), 1.88-1.70 (m, 2H). Mass (m/z): 392.2 [M+H]⁺.

### Compound 188

### tert-butyl 2-methyl-3-oxo-6-((4-(4-(trifluoromethyl)piperidin-1-yl)phenyl) amino)-2,3-dihydro-1H-indazole-1-carboxylate

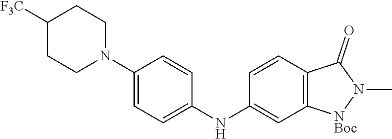 [SMILES: Cn1c(=O)c2ccc(Nc3ccc(N4CCC(C(F)(F)F)CC4)cc3)cc2n1C(=O)OC(C)(C)C]

The title compound 188 (26.4 mg) was prepared in a total yield of 53.8% as a yellow solid from 4-(4-(trifluoromethyl)piperidin-1-yl)aniline (31.8 mg, 0.13 mmol), tert-butyl 6-bromo-2-methyl-3-oxo-2,3-dihydro-1H-indazole-1-carboxylate (32.8 mg, 0.1 mmol), Pd₂(dba)₃ (1.84 mg, 0.002 mmol), X-Phos (4.76 mg, 0.01 mmol) and Cs₂CO₃ (48.8 mg, 0.15 mmol) according to the procedure for compound 1. ¹H NMR (400 MHz, Chloroform-d) δ 7.62 (d, J=8.4 Hz, 1H), 7.21 (s, 1H), 7.16-7.12 (m, 2H), 6.95 (d, J=8.4 Hz, 2H), 6.72 (m, 1H), 3.75-3.68 (m, 2H), 3.53 (s, 3H), 2.77-2.62 (m, 2H), 2.17 (m, 1H), 2.03-1.95 (m, 2H), 1.84-1.74 (m, 2H), 1.53 (s, 9H). Mass (m/z): 491.3 [M+H]⁺.

### Compound 189

### cyclopropyl(1,1-dimethyl-5-((4-(4-(trifluoromethyl)piperidin-1-yl)phenyl) amino)isoindolin-2-yl)methanone

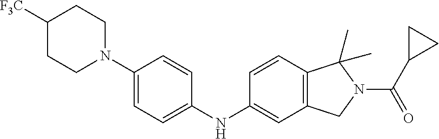 [SMILES: CC1(C)c2ccc(Nc3ccc(N4CCC(C(F)(F)F)CC4)cc3)cc2CN1C(=O)C1CC1]

The title compound 189 (17.7 mg) was prepared in a total yield of 77.3% as a yellow solid from 4-(4-(trifluoromethyl)piperidin-1-yl)aniline (15.9 mg, 0.065 mmol), (5-bromo-1,1-dimethylisoindolin-2-yl) (cyclopropyl) methanone (14.7 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.12-7.00 (m, 3H), 6.96-6.88 (m, 2H), 6.89-6.83 (m, 1H), 6.73 (s, 1H), 4.92 (s, 2H), 3.72-3.62 (m, 2H), 2.73-2.59 (m, 2H), 2.14 (m, 1H), 2.03-1.91 (m, 2H), 1.83-1.75 (m, 3H), 1.70 (s, 6H), 1.06-0.97 (m, 2H), 0.83-0.72 (m, 2H). Mass (m/z): 458.1 [M+H]⁺.

### Compound 190

### cyclopropyl(5-((4-(4-(trifluoromethyl)piperidin-1-yl)phenyl)amino) isoindolin-2-yl)methanone

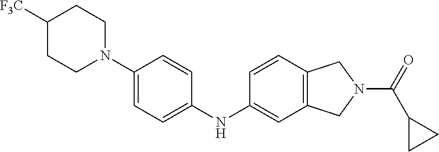 [SMILES: O=C(C1CC1)N1Cc2ccc(Nc3ccc(N4CCC(C(F)(F)F)CC4)cc3)cc2C1]

The title compound 190 (16.5 mg) was prepared in a total yield of 76.7% as a yellow solid from 4-(4-(trifluoromethyl)piperidin-1-yl)aniline (15.9 mg, 0.065 mmol), (5-bromoisoindolin-2-yl)(cyclopropyl)methanone (13.3 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.19-6.72 (m, 7H), 4.91 (s, 2H), 4.72 (s, 2H), 3.73-3.62 (m, 2H), 2.78-2.57 (m, 2H), 2.14 (m, 1H), 2.04-1.93 (m, 2H), 1.86-1.64 (m, 3H), 1.09-1.03 (m, 2H), 0.86-0.80 (m, 2H). Mass (m/z): 430.2 [M+H]⁺.

### Compound 191

### 2-methyl-6-((4-(4-(trifluoromethyl)piperidin-1-yl)phenyl)amino)-1,2-dihydro-3H-indazol-3-one

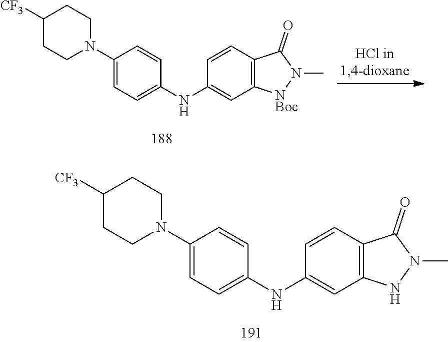 [SMILES: Cn1c(=O)c2ccc(Nc3ccc(N4CCC(C(F)(F)F)CC4)cc3)cc2n1C(=O)OC(C)(C)C ; Cn1[nH]c2cc(Nc3ccc(N4CCC(C(F)(F)F)CC4)cc3)ccc2c1=O]

A solution of tert-butyl 2-methyl-3-oxo-6-((4-(4-(trifluoromethyl) piperidin-1-yl) phenyl)amino)-2,3-dihydro-1H-indazole-1-carboxylate (12.3 mg, 0.025 mmol) in 1 mL of a solution of HCl in 1,4-dioxane was stirred for 30 mins at r.t. and concentrated. 5 ml water was added. The pH of the filtrate was adjusted to 8-9 with sodium carbonate solution. Then the mixture was extracted by DCM (3 mL×3). The combined organic layers were washed with water (5 mL), dried over Na₂SO₄ and concentrated. The residue was purified by perp-TLC (MeOH/DCM=1/10) to afford the desired product as a yellow solid (8.3 mg, 85.2%). ¹H NMR (400 MHz, DMSO-d₆) δ 7.36 (d, J=8.8 Hz, 1H), 7.06 (d, J=8.8 Hz, 2H), 6.96 (d, J=8.8 Hz, 2H), 6.62 (d, J=8.8 Hz, 1H), 6.52 (s, 1H), 3.73-3.65 (m, 2H), 3.22 (s, 3H), 2.73-2.61 (m, 2H), 2.11 (m, 1H), 1.93-1.85 (m, 2H), 1.65-1.51 (m, 2H). Mass (m/z): 391.2 [M+H]⁺.

### Compound 192

### 5-((3,5-difluoro-4-(4-methylpiperidin-1-yl)phenyl)amino)-2-methylisoindolin-1-one

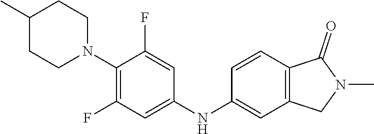

The title compound 192 (37.3 mg) was prepared in a total yield of 45.4% as a white solid from 3,5-difluoro-4-(4-methylpiperidin-1-yl)aniline (65 mg, 0.288 mmol), 5-bromo-2-methylisoindolin-1-one (50 mg, 0.221 mmol), Pd₂(dba)₃ (2 mg, 0.002 mmol), X-phos (6 mg, 0.01 mmol) and Cs₂CO₃ (109 mg, 0.332 mmol) according to the procedure for compound 1. ¹H NMR (400 MHz, DMSO-d₆) δ 7.61-7.50 (m, 1H), 7.46 (d, J=8.4 Hz, 1H), 7.22 (d, J=2.0 Hz, 1H), 7.09 (dd, J=8.4, 2.0 Hz, 1H), 6.82-6.69 (m, 2H), 2.98 (s, 3H), 2.96-2.86 (m, 4H), 1.56 (dd, J=12.8, 3.2 Hz, 2H), 1.37 (dqd, J=13.2, 8.0, 6.8, 2.8 Hz, 1H), 1.18 (tq, J=11.6, 4.8 Hz, 2H), 0.88 (d, J=6.4 Hz, 3H). Mass (m/z): 372.3 [M+H]⁺.

### Compound 193

### 5-((3,5-difluoro-4-(4-methylpiperidin-1-yl)phenyl)amino)-2-ethylisoindolin-1-one

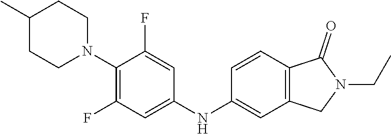 [SMILES: CCN1Cc2cc(Nc3cc(F)c(N4CCC(C)CC4)c(F)c3)ccc2C1=O]

The title compound 193 (27.1 mg) was prepared in a total yield of 33.8% as a white solid from 3,5-difluoro-4-(4-methylpiperidin-1-yl)aniline (61 mg, 0.271 mmol), 5-bromo-2-ethylisoindolin-1-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, DMSO-d₆) δ 8.77 (s, 1H), 7.49 (dd, J=8.0, 1.2 Hz, 1H), 7.21 (d, J=1.6 Hz, 1H), 7.05 (dt, J=8.0, 1.6 Hz, 1H), 6.76-6.66 (m, 2H), 4.36 (s, 2H), 3.54-3.40 (m, 2H), 3.02-2.88 (m, 4H), 1.68-1.55 (m, 2H), 1.48-1.34 (m, 1H), 1.21 (qd, J=11.6, 4.4 Hz, 2H), 1.12 (td, J=7.2, 1.2 Hz, 3H), 0.91 (dd, J=6.4, 1.2 Hz, 3H). Mass (m/z): 386.3 [M+H]⁺.

### Compound 194

### 6-methyl-3-((4-(4-(trifluoromethyl)piperidin-1-yl)phenyl)amino)-5,6-dihydro-7H-pyrrolo[3,4-b]pyridin-7-one

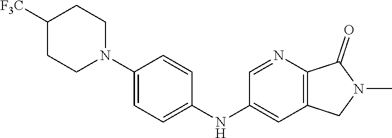 [SMILES: CN1Cc2cc(Nc3ccc(N4CCC(C(F)(F)F)CC4)cc3)cnc2C1=O]

The title compound 194 (13.2 mg) was prepared in a total yield of 67.7% as a yellow solid from 4-(4-(trifluoromethyl)piperidin-1-yl)aniline (15.9 mg, 0.065 mmol), 3-bromo-6-methyl-5,6-dihydro-7H-pyrrolo[3,4-b]pyridin-7-one (11.3 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, DMSO-d₆) δ 8.30 (s, 1H), 7.17 (s, 1H), 7.12 (d, J=8.8 Hz, 2H), 6.96 (d, J=8.8 Hz, 2H), 4.24 (s, 2H), 3.76-3.69 (m, 2H), 3.19 (s, 3H), 2.77-2.67 (m, 2H), 2.19 (m, 1H), 2.04-1.95 (m, 2H), 1.85-1.70 (m, 2H). Mass (m/z): 391.2 [M+H]⁺.

### Compound 195

### 2-ethyl-N-(5-fluoro-6-(piperidin-1-yl)pyridin-3-yl)isoindolin-5-amine

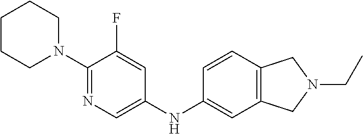 [SMILES: CCN1Cc2ccc(Nc3cnc(N4CCCCC4)c(F)c3)cc2C1]

The title compound 195 (15.0 mg) was prepared in a total yield of 88.2% as a yellow solid from 5-fluoro-6-(piperidin-1-yl)pyridin-3-amine (12.7 mg, 0.065 mmol), 5-bromo-2-ethylisoindoline (11.3 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.84 (d, J=2.0 Hz, 1H), 7.10-6.99 (m, 2H), 6.79 (d, J=8.4 Hz, 1H), 6.74 (s, 1H), 4.06 (s, 2H), 4.02 (s, 2H), 3.33-3.23 (m, 4H), 2.93 (q, J=7.2 Hz, 2H), 1.75-1.65 (m, 4H), 1.65-1.56 (m, 2H), 1.28 (t, J=7.2 Hz, 3H). Mass (m/z): 341.2 [M+H]⁺.

### Compound 196

### 2-(cyclopropylmethyl)-N-(5-fluoro-6-(piperidin-1-yl)pyridin-3-yl) isoindolin-5-amine

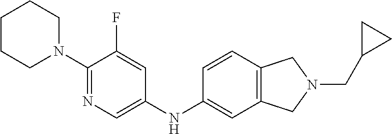 [SMILES: Fc1cc(Nc2ccc3c(c2)CN(CC2CC2)C3)cnc1N1CCCCC1]

The title compound 196 (15.3 mg) was prepared in a total yield of 83.4% as a yellow solid from 5-fluoro-6-(piperidin-1-yl)pyridin-3-amine (12.7 mg, 0.065 mmol), 5-bromo-2-(cyclopropylmethyl)isoindoline (12.6 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.83 (d, J=2.0 Hz, 1H), 7.09-6.94 (m, 2H), 6.80 (d, J=8.4 Hz, 1H), 6.71 (s, 1H), 4.18 (s, 2H), 4.12 (s, 2H), 3.30-3.21 (m, 4H), 2.82 (d, J=6.8 Hz, 2H), 1.71-1.63 (m, 4H), 1.63-1.55 (m, 2H), 1.11 (m, 1H), 0.67-0.58 (m, 2H), 0.33-0.27 (m, 2H). Mass (m/z): 367.3 [M+H]⁺.

### Compound 197

### 2-(tert-butyl)-5-((4-(4-(trifluoromethyl)piperidin-1-yl)phenyl)amino) isoindolin-1-one

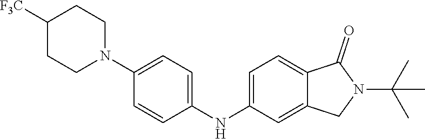 [SMILES: CC(C)(C)N1Cc2cc(Nc3ccc(N4CCC(C(F)(F)F)CC4)cc3)ccc2C1=O]

The title compound 197 (32.6 mg) was prepared in a yield of 18.45% as a pale yellow powder from 4-(4-(trifluoromethyl)piperidin-1-yl)aniline (100 mg, 0.41 mmol) and 5-bromo-2-(tert-butyl)isoindolin-1-one (121 mg, 0.45 mmol) according to the procedure for compound 1. ¹H NMR (400 MHz, Methanol-d₄) δ 7.66-7.58 (m, 1H), 7.45 (d, J=8.4 Hz, 1H), 7.34-7.25 (m, 2H), 7.11 (d, J=8.4 Hz, 2H), 6.95-6.89 (m, 1H), 4.44 (d, J=0.9 Hz, 2H), 3.67 (d, J=12.5 Hz, 2H), 2.76-2.60 (m, 2H), 2.29 (dtq, J=16.7, 8.4, 4.3 Hz, 1H), 2.01-1.93 (m, 2H), 1.74 (td, J=12.6, 4.1 Hz, 2H), 1.53 (s, 9H). Mass (m/z): 432.5 [M+H]⁺.

### Compound 198

### 2-(2-(2-methoxyethoxy)ethyl)-5-((4-(4-(trifluoromethyl)piperidin-1-yl) phenyl)amino)isoindolin-1-one

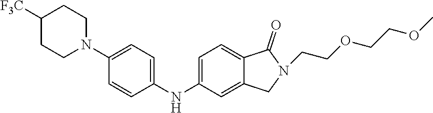 [SMILES: COCCOCCN1Cc2cc(Nc3ccc(N4CCC(C(F)(F)F)CC4)cc3)ccc2C1=O]

The title compound 198 (41.6 mg) was prepared in a yield of 21.28% as a white powder from 4-(4-(trifluoromethyl)piperidin-1-yl)aniline (100 mg, 0.41 mmol) and 5-bromo-2-(2-(2-methoxyethoxy)ethyl)isoindolin-1-one (141 mg, 0.45 mmol) according to the procedure for compound 1. ¹H NMR (400 MHz, Methanol-d₄) δ 7.53 (d, J=8.4 Hz, 1H), 7.16-7.08 (m, 2H), 7.03-6.89 (m, 4H), 4.44 (s, 2H), 3.73-3.69 (m, 4H), 3.69-3.63 (m, 2H), 3.62-3.59 (m, 2H), 3.53-3.49 (m, 2H), 2.68 (s, 2H), 2.27 (tdt, J=12.3, 7.8, 3.9 Hz, 1H), 2.00-1.92 (m, 2H), 1.71 (qd, J=12.6, 4.1 Hz, 2H). Mass (m/z): 478.3 [M+H]⁺.

### Compound 199

### 2-benzyl-5-((4-(4-(trifluoromethyl)piperidin-1-yl)phenyl)amino) isoindolin-1-one

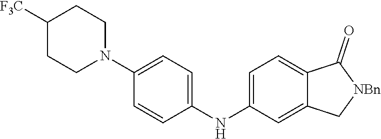 [SMILES: O=C1c2ccc(Nc3ccc(N4CCC(C(F)(F)F)CC4)cc3)cc2CN1Cc1ccccc1]

The title compound 199 (75.2 mg) was prepared in a yield of 39.37% as a white powder from 4-(4-(trifluoromethyl)piperidin-1-yl)aniline (100 mg, 0.41 mmol) and 2-benzyl-5-bromoisoindolin-1-one (123 mg, 0.45 mmol) according to the procedure for compound 1. ¹H NMR (400 MHz, Methanol-d₄) δ 7.57 (d, J=9.0 Hz, 1H), 7.39-7.31 (m, 3H), 7.31-7.25 (m, 3H), 7.14-7.08 (m, 2H), 7.03-6.98 (m, 2H), 6.96 (dq, J=4.6, 2.1 Hz, 2H), 4.74 (s, 2H), 4.23 (s, 2H), 3.72-3.65 (m, 2H), 2.69 (td, J=12.4, 2.5 Hz, 2H), 2.29 (dtd, J=16.1, 8.1, 3.9 Hz, 1H), 1.97 (dt, J=12.9, 3.0 Hz, 2H), 1.72 (qd, J=12.6, 4.2 Hz, 2H). Mass (m/z): 466.3 [M+H]⁺.

### Compound 200

### 2-(3-(dimethylamino)propyl)-5-((4-(4-(trifluoromethyl)piperidin-1-yl) phenyl)amino)isoindolin-1-one

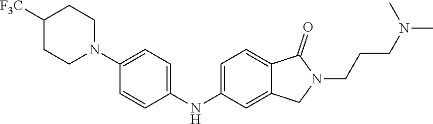 [SMILES: CN(C)CCCN1Cc2cc(Nc3ccc(N4CCC(C(F)(F)F)CC4)cc3)ccc2C1=O]

The title compound 200 (52.9 mg) was prepared in a yield of 28.06% as a white powder from 4-(4-(trifluoromethyl)piperidin-1-yl)aniline (100 mg, 0.41 mmol) and 5-bromo-2-(3-(dimethylamino)propyl)isoindolin-1-one (121 mg, 0.41 mmol) according to the procedure for compound 1. ¹H NMR (400 MHz, Methanol-d₄) δ 7.51 (dd, J=8.4, 0.5 Hz, 1H), 7.14-7.09 (m, 2H), 7.00 (dt, J=4.3, 1.5 Hz, 2H), 6.98 (d, J=2.2 Hz, 1H), 6.95 (dd, J=8.4, 2.0 Hz, 1H), 4.39 (s, 2H), 3.73-3.58 (m, 4H), 2.96-2.86 (m, 2H), 2.71 (s, 8H), 2.27 (dtq, J=17.0, 8.5, 4.2 Hz, 1H), 2.08-1.91 (m, 4H), 1.77-1.63 (m, 2H). Mass (m/z): 461.8 [M+H]⁺.

### Compound 201

### 2-methyl-5-((4-(4-(trifluoromethyl)piperidin-1-yl)phenyl)amino) isoindoline-1,3-dione

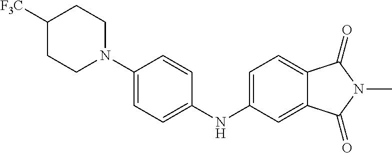 [SMILES: CN1C(=O)c2ccc(Nc3ccc(N4CCC(C(F)(F)F)CC4)cc3)cc2C1=O]

The title compound 201 (16.3 mg) was prepared in a total yield of 80.9% as a yellow solid from 4-(4-(trifluoromethyl)piperidin-1-yl)aniline (15.9 mg, 0.065 mmol), 5-bromo-2-methylisoindoline-1,3-dione (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.45 (m, 1H), 7.15-6.88 (m, 3H), 6.87-6.70 (m, 3H), 3.67-3.43 (m, 2H), 2.94 (s, 3H), 2.65-2.45 (m, 2H), 2.01 (m, 1H), 1.87-1.77 (m, 2H), 1.67-1.52 (m, 2H). Mass (m/z): 404.2 [M+H]⁺.

### Compound 202

### 7-fluoro-2-methyl-5-((4-(4-(trifluoromethyl)piperidin-1-yl)phenyl)amino) isoindolin-1-one

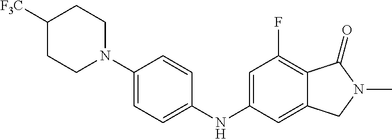 [SMILES: CN1Cc2cc(Nc3ccc(N4CCC(C(F)(F)F)CC4)cc3)cc(F)c2C1=O]

The title compound 202 (17.7 mg) was prepared in a total yield of 86.8% as a yellow solid from 4-(4-(trifluoromethyl)piperidin-1-yl)aniline (15.9 mg, 0.065 mmol), 5-bromo-7-fluoro-2-methylisoindolin-1-one (12.2 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.10 (d, J=8.4 Hz, 2H), 6.94 (d, J=8.4 Hz, 2H), 6.82-6.76 (m, 2H), 4.22 (s, 2H), 3.77-3.66 (m, 2H), 3.09 (s, 3H), 2.78-2.62 (m, 2H), 2.17 (m, 1H), 2.04-1.95 (m, 2H), 1.83-1.71 (m, 2H). Mass (m/z): 408.2 [M+H]⁺.

### Compound 203

### 7-chloro-2-methyl-5-((4-(4-(trifluoromethyl)piperidin-1-yl)phenyl)amino) isoindolin-1-one

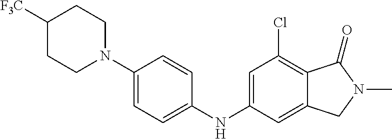 [SMILES: CN1Cc2cc(Nc3ccc(N4CCC(C(F)(F)F)CC4)cc3)cc(Cl)c2C1=O]

The title compound 203 (18.6 mg) was prepared in a total yield of 87.9% as a yellow solid from 4-(4-(trifluoromethyl)piperidin-1-yl)aniline (15.9 mg, 0.065 mmol), 5-bromo-7-chloro-2-methylisoindolin-1-one (13.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.13-7.07 (m, 2H), 6.99-6.92 (m, 2H), 6.84-6.77 (m, 2H), 4.23 (s, 2H), 3.72-3.64 (m, 2H), 3.08 (s, 3H), 2.75-2.63 (m, 2H), 2.19 (m, 1H), 2.01-1.93 (d, J=3.8 Hz, 2H), 1.80-1.66 (m, 2H). Mass (m/z): 424.1 [M+H]⁺.

### Compound 204

### 5-((3-fluoro-4-(4-(trifluoromethyl)piperidin-1-yl)phenyl)amino)-2-methylisoindolin-1-one

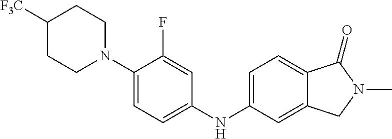 [SMILES: CN1Cc2cc(Nc3ccc(N4CCC(C(F)(F)F)CC4)c(F)c3)ccc2C1=O]

The title compound 204 (16.2 mg) was prepared in a total yield of 79.4% as a yellow solid from 3-fluoro-4-(4-(trifluoromethyl)piperidin-1-yl)aniline (17.0 mg, 0.065 mmol), 5-bromo-2-methylisoindolin-1-one (11.3 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.62 (d, J=8.4 Hz, 1H), 7.02-6.85 (m, 4H), 6.51 (s, 1H), 4.25 (s, 2H), 3.54-3.41 (m, 2H), 3.13 (s, 3H), 2.71-2.62 (m, 2H), 2.14 (m, 1H), 2.01-1.92 (m, 2H), 1.89-1.76 (m, 2H). Mass (m/z): 408.2 [M+H]⁺.

### Compound 205

### N-(tert-butyl)-5-((4-(4-(trifluoromethyl)piperidin-1-yl)phenyl)amino) isoindoline-2-carboxamide

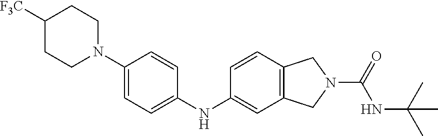 [SMILES: CC(C)(C)NC(=O)N1Cc2ccc(Nc3ccc(N4CCC(C(F)(F)F)CC4)cc3)cc2C1]

The title compound 205 (19.0 mg) was prepared in a total yield of 82.6% as a yellow solid from 4-(4-(trifluoromethyl)piperidin-1-yl)aniline (15.9 mg, 0.065 mmol), 5-bromo-N-(tert-butyl)isoindoline-2-carboxamide (14.9 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.14-6.81 (m, 7H), 4.54 (s, 4H), 3.69-3.57 (m, 2H), 2.74-2.62 (m, 2H), 2.27 (m, 1H), 2.02-1.93 (m, 2H), 1.79-1.67 (m, 2H), 1.39 (s, 9H). Mass (m/z): 461.3 [M+H]⁺.

### Compound 206

### 2-(1-methyl-1H-imidazol-4-yl)-5-((4-(4-(trifluoromethyl)piperidin-1-yl) phenyl)amino)isoindolin-1-one

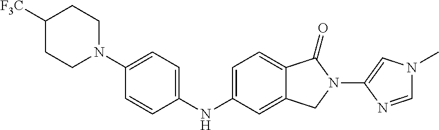 [SMILES: Cn1cnc(N2Cc3cc(Nc4ccc(N5CCC(C(F)(F)F)CC5)cc4)ccc3C2=O)c1]

The title compound 206 (7.1 mg) was prepared in a yield of 7.61% as a yellow powder from 4-(4-(trifluoromethyl)piperidin-1-yl)aniline (50 mg, 0.20 mmol) and 5-bromo-2-(1-methyl-1H-imidazol-4-yl)isoindolin-1-one (60 mg, 0.20 mmol) according to the procedure for compound 1. ¹H NMR (400 MHz, Chloroform-d) δ 7.75-7.68 (m, 1H), 7.52 (s, 1H), 7.40 (tdd, J=8.6, 6.4, 1.7 Hz, 4H), 7.29 (d, J=3.4 Hz, 1H), 7.04 (td, J=7.7, 2.6 Hz, 1H), 7.00-6.95 (m, 1H), 6.69 (ddd, J=15.6, 7.6, 1.6 Hz, 1H), 4.83 (s, 2H), 2.12 (d, J=37.6 Hz, 2H), 2.04 (s, 1H), 1.99 (s, 2H), 1.78 (s, 2H), 1.73 (s, 3H), 1.56 (s, 1H), 1.25 (t, J=7.1 Hz, 1H). Mass (m/z): 456.2 [M+H]⁺.

### Compound 207

### N-(4-(2,6-dimethylmorpholino) phenyl)-1-methyl-1H-benzo[d][1,2,3]triazol-5-amine

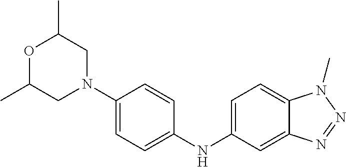 [SMILES: CC1CN(c2ccc(Nc3ccc4c(c3)nnn4C)cc2)CC(C)O1]

To a solution of 4-(2,6-dimethylmorpholin-4-yl)aniline (107 mg, 0.518 mmol), 5-bromo-1-methyl-1,2,3-benzotriazole (109 mg, 0.518 mmol) in toluene (2 mL) was added Cs₂CO₃ (338 mg, 1.04 mmol), Pd₂(dba)₃ (48 mg, 0.052 mmol) and Ru-Phos (48 mg, 0.104 mmol). The reaction mixture was stirred at 100° C. for 18 h under N₂ atmosphere. The resulting mixture was concentrated under reduced pressure. The residue was purified by column chromatography (EA:PE=1:3) to give the target compound (61.8 mg, 34.55%) as a green solid. MS (ESI) m/z: 338 [M+H]⁺.

¹H NMR (400 MHz, DMSO-d6) δ 7.99 (s, 1H), 7.65 (d, J=8.8 Hz, 1H), 7.28 (s, 1H), 7.19 (dd, J=2.0, 8.8 Hz, 1H), 7.06 (d, J=8.8 Hz, 2H), 6.92 (d, J=8.8 Hz, 2H), 4.22 (s, 3H), 3.70-3.68 (m, 2H), 3.47 (d, J=10.4 Hz, 2H), 2.24-2.18 (m, 2H), 1.16 (d, J=6.0 Hz, 6H).

### Compound 208

### 2-(1-methylpiperidin-4-yl)-5-((4-(4-(trifluoromethyl)piperidin-1-yl) phenyl)amino)isoindolin-1-one

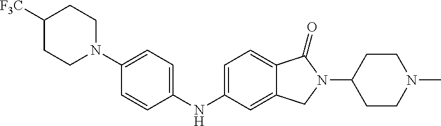 [SMILES: CN1CCC(N2Cc3cc(Nc4ccc(N5CCC(C(F)(F)F)CC5)cc4)ccc3C2=O)CC1]

The title compound 208 (66.5 mg) was prepared in a yield of 34.37% as a white powder from 4-(4-(trifluoromethyl)piperidin-1-yl)aniline (100 mg, 0.41 mmol) and 5-bromo-2-(1-methylpiperidin-4-yl)isoindolin-1-one (127 mg, 0.41 mmol) according to the procedure for compound 1. ¹H NMR (400 MHz, Methanol-d₄) δ 7.50 (d, J=8.4 Hz, 1H), 7.10 (d, J=8.4 Hz, 2H), 7.04-6.86 (m, 4H), 4.39-4.28 (m, 3H), 3.75-3.59 (m, 2H), 3.59-3.47 (m, 2H), 3.13 (td, J=12.9, 2.9 Hz, 2H), 2.85 (s, 3H), 2.64 (s, 2H), 2.34-2.11 (m, 3H), 2.10-1.87 (m, 4H), 1.67 (qd, J=12.6, 4.1 Hz, 2H). Mass (m/z): 473.2 [M+H]⁺.

### Compound 209

### 2-cyclopropyl-5-((4-(4-(trifluoromethyl)piperidin-1-yl)phenyl)amino) isoindolin-1-one

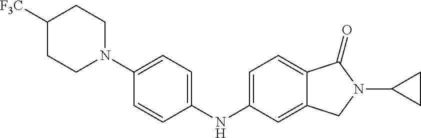 [SMILES: O=C1c2ccc(Nc3ccc(N4CCC(C(F)(F)F)CC4)cc3)cc2CN1C1CC1]

The title compound 209 (67.2 mg) was prepared in a yield of 39.51% as a pale yellow powder from 4-(4-(trifluoromethyl)piperidin-1-yl)aniline (100 mg, 0.41 mmol) and 5-bromo-2-cyclopropylisoindolin-1-one (103 mg, 0.41 mmol) according to the procedure for compound 1. ¹H NMR (400 MHz, DMSO-d₆) δ 8.30 (s, 1H), 7.44-7.34 (m, 1H), 7.08-7.03 (m, 2H), 6.98-6.93 (m, 2H), 6.92 (q, J=1.4, 0.8 Hz, 1H), 6.88 (dt, J=8.3, 2.0 Hz, 1H), 4.22 (s, 2H), 3.69 (d, J=12.5 Hz, 2H), 2.83 (tt, J=6.8, 4.3 Hz, 1H), 2.73-2.60 (m, 2H), 2.46 (d, J=3.2 Hz, 1H), 1.93-1.82 (m, 2H), 1.57 (qd, J=12.5, 4.1 Hz, 2H), 0.79-0.65 (m, 4H). Mass (m/z): 473.2 [M+H]⁺.

### Compound 210

### 2-isopropyl-5-((4-(4-(trifluoromethyl)piperidin-1-yl)phenyl)amino) isoindolin-1-one

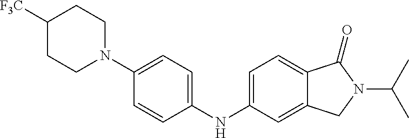 [SMILES: CC(C)N1Cc2cc(Nc3ccc(N4CCC(C(F)(F)F)CC4)cc3)ccc2C1=O]

The title compound 210 (32.4 mg) was prepared in a yield of 18.96% as a pale yellow powder from 4-(4-(trifluoromethyl)piperidin-1-yl)aniline (100 mg, 0.41 mmol) and 5-bromo-2-isopropylisoindolin-1-one (104 mg, 0.41 mmol) according to the procedure for compound 1. ¹H NMR (400 MHz, Methanol-d₄) δ 7.51 (dd, J=8.4, 0.9 Hz, 1H), 7.29 (dt, J=15.2, 7.2 Hz, 2H), 7.14-7.10 (m, 2H), 7.04-6.99 (m, 3H), 4.33 (s, 2H), 3.74-3.63 (m, 2H), 2.74-2.66 (m, 2H), 2.30 (dtt, J=15.8, 7.6, 3.9 Hz, 1H), 1.98 (d, J=12.6 Hz, 2H), 1.74 (td, J=12.5, 4.3 Hz, 3H), 1.28 (dd, J=6.8, 1.1 Hz, 6H). Mass (m/z): 418.7 [M+H]⁺.

### Compound 211

### 5-((4-(4,4-dimethylpiperidin-1 yl)phenyl)amino)-2-methylisoindolin-1-one

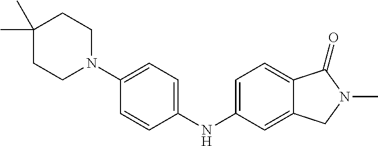

The title compound 211 (35.6 mg) was prepared in a yield of 20.3% as a yellow powder from 4-(4,4-dimethylpiperidin-1-yl)aniline (107 mg, 0.5 mmol), 5-bromo-2-methylisoindolin-1-one (87 mg, 0.38 mmol), Pd(dppf)Cl₂ (5.6 mg, 7.6 umol), Xantphos (8.8 mg, 15.2 umol), Cs₂CO₃ (245 mg, 0.75 mmol) and 1,4-dioxane (3.0 mL) according to the procedure for compound 1. (35.6 mg, 20.3%). ¹H NMR (400 MHz, Methanol-d₄) δ 7.49 (d, J=8.4 Hz, 1H), 7.13-7.08 (m, 2H), 7.02-6.96 (m, 3H), 6.92 (dd, J=8.4, 2.1 Hz, 1H), 4.33 (s, 2H), 3.15-3.05 (m, 7H), 1.57-1.52 (m, 4H), 1.00 (s, 6H). Mass (m/z): 350.3 [M+H]⁺.

### Compound 212

### 7-fluoro-2-methyl-5-((4-(4-(trifluoromethyl)piperidin-1-yl)phenyl)amino) isoindolin-1-one

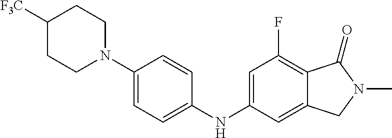 [SMILES: CN1Cc2cc(Nc3ccc(N4CCC(C(F)(F)F)CC4)cc3)cc(F)c2C1=O]

The title compound 212(17.7 mg) was prepared in a total yield of 86.8% as a yellow solid from 4-(4-(trifluoromethyl)piperidin-1-yl)aniline (15.9 mg, 0.065 mmol), 5-bromo-7-fluoro-2-methylisoindolin-1-one (12.2 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.10 (d, J=8.4 Hz, 2H), 6.94 (d, J=8.4 Hz, 2H), 6.82-6.76 (m, 2H), 4.22 (s, 2H), 3.77-3.66 (m, 2H), 3.09 (s, 3H), 2.78-2.62 (m, 2H), 2.17 (m, 1H), 2.04-1.95 (m, 2H), 1.83-1.71 (m, 2H). Mass (m/z): 408.2 [M+H]⁺.

### Compound 213

### 7-chloro-2-methyl-5-((4-(4-(trifluoromethyl)piperidin-1-yl)phenyl) amino)isoindolin-1-one

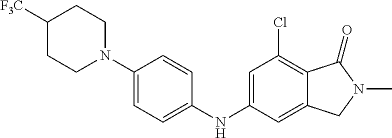 [SMILES: CN1Cc2cc(Nc3ccc(N4CCC(C(F)(F)F)CC4)cc3)cc(Cl)c2C1=O]

The title compound 213 (18.6 mg) was prepared in a total yield of 87.9% as a yellow solid from 4-(4-(trifluoromethyl)piperidin-1-yl)aniline (15.9 mg, 0.065 mmol), 5-bromo-7-chloro-2-methylisoindolin-1-one (13.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.13-7.07 (m, 2H), 6.99-6.92 (m, 2H), 6.84-6.77 (m, 2H), 4.23 (s, 2H), 3.72-3.64 (m, 2H), 3.08 (s, 3H), 2.75-2.63 (m, 2H), 2.19 (m, 1H), 2.01-1.93 (d, J=3.8 Hz, 2H), 1.80-1.66 (m, 2H). Mass (m/z): 424.1 [M+H]⁺.

### Compound 214

### 5-((3-fluoro-4-(4-(trifluoromethyl)piperidin-1-yl)phenyl)amino)-2-methylisoindolin-1-one

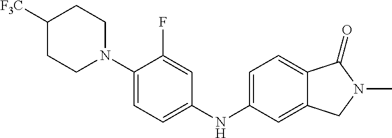 [SMILES: CN1Cc2cc(Nc3ccc(N4CCC(C(F)(F)F)CC4)c(F)c3)ccc2C1=O]

The title compound 214 (16.2 mg) was prepared in a total yield of 79.4% as a yellow solid from 3-fluoro-4-(4-(trifluoromethyl)piperidin-1-yl)aniline (17.0 mg, 0.065 mmol), 5-bromo-2-methylisoindolin-1-one (11.3 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.62 (d, J=8.4 Hz, 1H), 7.02-6.85 (m, 4H), 6.51 (s, 1H), 4.25 (s, 2H), 3.54-3.41 (m, 2H), 3.13 (s, 3H), 2.71-2.62 (m, 2H), 2.14 (m, 1H), 2.01-1.92 (m, 2H), 1.89-1.76 (m, 2H). Mass (m/z): 408.2 [M+H]⁺.

### Compound 215

### 2-(3,3-difluoroallyl)-5-((4-(4-(trifluoromethyl)piperidin-1-yl)phenyl) amino)isoindolin-1-one

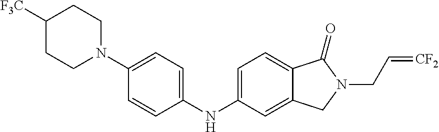

The title compound 215 (23.5 mg) was prepared in a yield of 12.72% as a pale yellow powder from 4-(4-(trifluoromethyl)piperidin-1-yl)aniline (100 mg, 0.41 mmol) and 5-bromo-2-(3,3-difluoroallyl)isoindolin-1-one (118 mg, 0.41 mmol) according to the procedure for compound 1. ¹H NMR (DMSO-d₆) δ: 8.35 (s, 1H), 7.42 (d, J=8.3 Hz, 1H), 7.12-7.02 (m, 2H), 7.01-6.93 (m, 3H), 6.90 (dd, J=8.4, 2.1 Hz, 1H), 4.76 (dtd, J=26.2, 7.7, 2.3 Hz, 1H), 4.30 (s, 2H), 4.07 (dt, J=7.7, 1.9 Hz, 2H), 3.69 (d, J=12.1 Hz, 2H), 2.66 (td, J=12.5, 2.5 Hz, 2H), 2.45 (td, J=12.4, 10.5, 6.3 Hz, 1H), 1.88 (d, J=12.3 Hz, 2H), 1.57 (qd, J=12.5, 4.1 Hz, 2H). 19F NMR (DMSO-d₆) δ : −72.49 (d, J=8.8 Hz), −86.58 (d, J=2.2 Hz), −86.69 (d, J=2.2 Hz), −88.75, −88.84 (d, J=15.8 Hz), −88.93. Mass (m/z): 452.3 [M+H]⁺.

### Compound 216

### tert-butyl 6-((3-fluoro-4-(4-(trifluoromethyl)piperidin-1-yl)phenyl)amino)-2-methyl-3-oxo-2,3-dihydro-1H-indazole-1-carboxylate

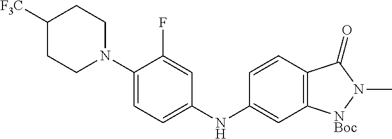 [SMILES: Cn1c(=O)c2ccc(Nc3ccc(N4CCC(C(F)(F)F)CC4)c(F)c3)cc2n1C(=O)OC(C)(C)C]

The title compound 216 (18.4 mg) was prepared in a total yield of 72.3% as a yellow solid from 3-fluoro-4-(4-(trifluoromethyl)piperidin-1-yl)aniline (17.0 mg, 0.065 mmol), tert-butyl 6-bromo-2-methyl-3-oxo-2,3-dihydro-1H-indazole-1-carboxylate (16.4 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.57 (d, J=8.4 Hz, 1H), 7.38 (s, 1H), 7.07-6.95 (m, 3H), 6.85 (d, J=8.4 Hz, 1H), 3.53 (s, 3H), 3.48-3.42 (m, 2H), 2.76-2.66 (m, 2H), 2.24 (m, 1H), 2.02-1.93 (m, 2H), 1.83-1.72 (m, 2H), 1.56 (s, 9H). Mass (m/z): 509.3 [M+H]⁺.

### Compound 217

### 2-methyl-5-((4-(piperidin-1-yl)phenyl)amino)isoindolin-1-one

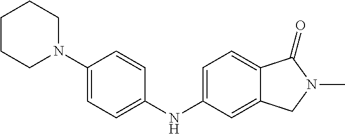 [SMILES: CN1Cc2cc(Nc3ccc(N4CCCCC4)cc3)ccc2C1=O]

The title compound 217 (13.6 mg) was prepared in a total yield of 85.0% as a yellow solid from 4-(piperidin-1-yl)aniline (11.4 mg, 0.065 mmol), 5-bromo-2-methylisoindolin-1-one (11.3 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.53 (d, J=8.4 Hz, 1H), 7.04 (d, J=8.8 Hz, 2H), 6.94-6.76 (m, 4H), 4.15 (s, 2H), 3.67-3.61 (m, 2H), 3.13-3.02 (m, 5H), 1.74-1.65 (m, 4H), 1.59-1.46 (m, 2H). Mass (m/z): 322.2 [M+H]⁺.

### Compound 218

### 1-ethyl-2-methyl-6-((4-(4-(trifluoromethyl)piperidin-1-yl)phenyl)amino)-1,2-dihydro-3H-indazol-3-one

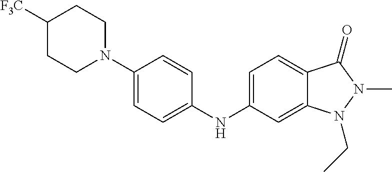 [SMILES: CCn1c2cc(Nc3ccc(N4CCC(C(F)(F)F)CC4)cc3)ccc2c(=O)n1C]

The title compound 218 (17.8 mg) was prepared in a total yield of 85.2% as a yellow solid from 4-(4-(trifluoromethyl)piperidin-1-yl)aniline (15.9 mg, 0.065 mmol), 6-bromo-1-ethyl-2-methyl-1,2-dihydro-3H-indazol-3-one (12.8 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.45 (d, J=8.8 Hz, 1H), 7.06 (d, J=8.8 Hz, 2H), 6.90 (d, J=8.8 Hz, 2H), 6.67 (d, J=8.8 Hz, 1H), 6.59 (s, 1H), 3.72 (q, J=6.8 Hz, 2H), 3.63-3.55 (m, 2H), 3.26 (s, 3H), 2.67-2.46 (m, 2H), 2.17 (m, 1H), 1.94-1.81 (m, 2H), 1.69-1.56 (m, 2H), 0.75 (t, J=6.8 Hz, 3H). Mass (m/z): 419.2 [M+H]⁺.

### Compound 219

### 6-((3-fluoro-4-(4-(trifluoromethyl)piperidin-1-yl)phenyl)amino)-2-methyl-1,2-dihydro-3H-indazol-3-one

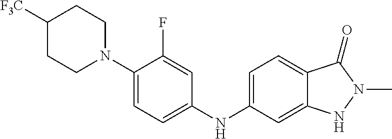 [SMILES: Cn1[nH]c2cc(Nc3ccc(N4CCC(C(F)(F)F)CC4)c(F)c3)ccc2c1=O]

The title compound 219 (8.9 mg) was prepared in a total yield of 87.0% as a yellow solid from tert-butyl 6-((3-fluoro-4-(4-(trifluoromethyl) piperidin-1-yl) phenyl)amino)-2-methyl-3-oxo-2,3-dihydro-1H-indazole-1-carboxylate (12.7 mg, 0.025 mmol) and HCl in 1,4-dioxane according to the procedure for compound 1. ¹H NMR (400 MHz, Methanol-d₄) δ 7.52 (d, J=8.8 Hz, 1H), 7.05-6.88 (m, 3H), 6.79-6.72 (m, 2H), 3.46-3.35 (m, 5H), 2.73-2.64 (m, 2H), 2.26 (m, 1H), 2.01-1.89 (m, 2H), 1.81-1.68 (m, 2H). Mass (m/z): 409.2 [M+H]⁺.

### Compound 220

### 1,3-dimethyl-5-((4-(4-(trifluoromethyl)piperidin-1-yl)phenyl)amino)-1,3-dihydro-2H-benzo[d]imidazol-2-one

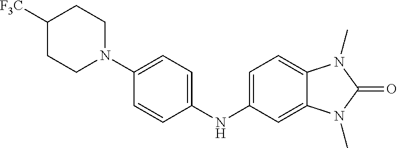 [SMILES: Cn1c(=O)n(C)c2cc(Nc3ccc(N4CCC(C(F)(F)F)CC4)cc3)ccc21]

The title compound 220 (32.9 mg) was prepared in a yield of 19.87% as a pale blue powder from 4-(4-(trifluoromethyl)piperidin-1-yl)aniline (100 mg, 0.41 mmol) and 5-bromo-1,3-dimethyl-1,3-dihydro-2H-benzo[d]imidazol-2-one (99 mg, 0.41 mmol) according to the procedure for compound 1. ¹H NMR (Methanol-d₄) δ 7.13-6.85 (m, 8H), 3.61 (dddd, J=24.5, 12.6, 5.5, 3.1 Hz, 4H), 3.15 (pd, J=6.7, 4.3 Hz, 1H), 2.74-2.60 (m, 3H), 2.32-2.14 (m, 2H), 1.82-1.65 (m, 2H), 1.16-1.05 (m, 2H), 0.96 (t, J=7.3 Hz, 1H). Mass (m/z): 405.4 [M+H]⁺.

### Compound 221

### 2-(4-methylcyclohexyl)-5-((4-(4-(trifluoromethyl)piperidin-1-yl)phenyl) amino)isoindolin-1-one

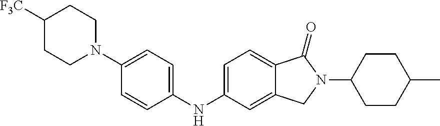 [SMILES: CC1CCC(N2Cc3cc(Nc4ccc(N5CCC(C(F)(F)F)CC5)cc4)ccc3C2=O)CC1]

The title compound 221 (14.6 mg) was prepared in a yield of 7.56% as a pale yellow powder from 4-(4-(trifluoromethyl)piperidin-1-yl)aniline (100 mg, 0.41 mmol) and 5-bromo-2-(4-methylcyclohexyl)isoindolin-1-one (126 mg, 0.41 mmol) according to the procedure for compound 1. ¹H NMR (Methanol-d₄) δ 7.86-6.26 (m, 7H), 4.07 (s, 2H), 1.97 (d, J=17.1 Hz, 3H), 1.87-1.65 (m, 8H), 1.64-1.47 (m, 5H), 1.13 (q, J=13.9, 13.1 Hz, 1H), 1.02 (dd, J=7.2, 2.4 Hz, 2H), 0.98-0.77 (m, 2H). Mass (m/z): 472.3 [M+H]⁺.

### Compound 222

### 2-methyl-5-((4-(3-(trifluoromethyl)pyrrolidin-1-yl)phenyl)amino) isoindolin-1-one

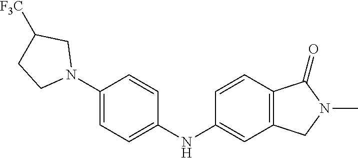 [SMILES: CN1Cc2cc(Nc3ccc(N4CCC(C(F)(F)F)C4)cc3)ccc2C1=O]

The title compound 222 (17.6 mg) was prepared in a total yield of 21.2% as a white solid from 4-(3-(trifluoromethyl)pyrrolidin-1-yl)aniline (66 mg, 0.288 mmol), 5-bromo-2-ethylisoindolin-1-one (50 mg, 0.221 mmol), Pd₂(dba)₃ (2 mg, 0.002 mmol), X-phos (6 mg, 0.01 mmol), Cs₂CO₃ (109 mg, 0.332 mmol) and 1,4-dioxane (5 mL) according to the procedure for compound 1. ¹H NMR (400 MHz, DMSO-d₆) δ 8.18 (s, 1H), 7.34 (d, J=8.4 Hz, 1H), 7.03 (d, J=8.4 Hz, 2H), 6.88-6.75 (m, 2H), 6.61 (d, J=8.4 Hz, 2H), 4.24 (s, 2H), 3.47 (t, J=8.8 Hz, 1H), 3.39-3.33 (m, 1H), 3.32-3.23 (m, 3H), 2.95 (s, 3H), 2.25 (dtd, J=12.8, 7.6, 5.0 Hz, 1H), 2.11-1.99 (m, 1H). Mass (m/z): 376.3 [M+H]⁺.

### Compound 223

### 5-((4-(3,3-dimethylazetidin-1-yl)phenyl)amino)-2-methylisoindolin-1-one

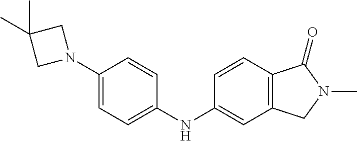

The title compound 223 (21.9 mg) was prepared in a total yield of 31.0% as a white solid from 4-(3,3-dimethylazetidin-1-yl)aniline (51 mg, 0.288 mmol), 5-bromo-2-ethylisoindolin-1-one (50 mg, 0.221 mmol), Pd₂(dba)₃ (2 mg, 0.002 mmol), X-phos (6 mg, 0.01 mmol), Cs₂CO₃ (109 mg, 0.332 mmol) and 1,4-dioxane (5 mL) according to the procedure for compound 1. ¹H NMR (400 MHz, DMSO-d₆) δ 8.21 (s, 1H), 7.34 (d, J=8.4 Hz, 1H), 6.98 (d, J=8.4 Hz, 2H), 6.84-6.76 (m, 2H), 6.41-6.34 (m, 2H), 4.24 (s, 2H), 3.46 (s, 3H), 3.32 (s, 1H), 2.95 (s, 3H), 1.25 (s, 6H). Mass (m/z): 322.3 [M+H]⁺.

### Compound 224

### 1,2-dimethyl-6-((4-(4-(trifluoromethyl)piperidin-1-yl)phenyl)amino)-1,2-dihydro-3H-indazol-3-one

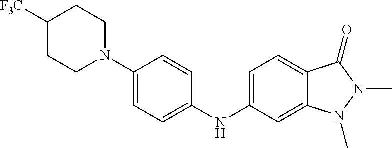 [SMILES: Cn1c(=O)c2ccc(Nc3ccc(N4CCC(C(F)(F)F)CC4)cc3)cc2n1C]

The title compound 224 (13.4 mg) was prepared in a total yield of 66.3% as a yellow solid from 4-(4-(trifluoromethyl)piperidin-1-yl)aniline (15.9 mg, 0.065 mmol), 6-bromo-1,2-dimethyl-1,2-dihydro-3H-indazol-3-one (12.1 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.52 (d, J=8.8 Hz, 1H), 7.24-6.53 (m, 6H), 3.75-3.62 (m, 2H), 3.35 (s, 3H), 3.16 (s, 3H), 2.78-2.63 (s, 2H), 2.18 (m, 1H), 2.03-1.93 (m, 2H), 1.83-1.66 (m, 2H). Mass (m/z): 405.2 [M+H]⁺.

### Compound 225

### 5-((3-chloro-4-(piperidin-1-yl)phenyl)amino)-2-methylisoindolin-1-one

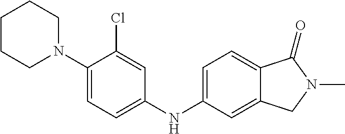 [SMILES: CN1Cc2cc(Nc3ccc(N4CCCCC4)c(Cl)c3)ccc2C1=O]

The title compound 225(12.5 mg) was prepared in a total yield of 70.4% as a yellow solid from 3-chloro-4-(piperidin-1-yl)aniline (13.7 mg, 0.065 mmol), 5-bromo-2-methylisoindolin-1-one (11.3 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.54 (d, J=8.4 Hz, 1H), 7.17 (s 1H), 7.09-6.93 (m, 4H), 4.30 (s, 2H), 3.11 (s, 3H), 2.95-2.87 (m, 4H), 1.77-1.67 (m, 4H), 1.60-1.51 (m, 2H). Mass (m/z): 356.1 [M+H]⁺.

### Compound 226

### 5-((3,5-dimethylphenyl)amino)-2-methylisoindolin-1-one

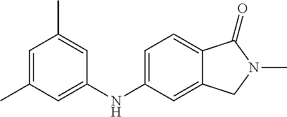

The title compound 226 (33.5 mg) was prepared in a total yield of 37.8% as a yellow solid from 3,5-dimethylaniline (52.2 mg, 0.43 mmol), 5-bromo-2-methylisoindolin-1-one (75 mg, 0.33 mmol), Pd(dppf)Cl₂ (4.8 mg, 6.6 umol), Xantphos (7.6 mg, 13.2 umol), and Cs₂CO₃ (163 mg, 0.50 mmol) according to the procedure for compound 1. ¹H NMR (400 MHz, Methanol-d₄) δ 7.53 (dd, J=8.4, 1.4 Hz, 1H), 7.11 (s, 1H), 7.02 (dt, J=8.4, 1.8 Hz, 1H), 6.80 (s, 2H), 6.64 (s, 1H), 4.37 (s, 2H), 3.13 (d, J=1.4 Hz, 3H), 2.26 (d, J=1.3 Hz, 6H). Mass (m/z): 267.2 [M+H]⁺.

### Compound 227

### 5-((4-(dimethylamino)phenyl)amino)-2-methylisoindolin-1-one

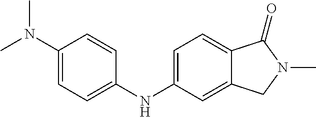

The title compound 227 (41.1 mg) was prepared in a total yield of 43.9% as a yellow solid from N,N-dimethyl-p-phenylenediamine (58.0 mg, 0.43 mmol), 5-bromo-2-methylisoindolin-1-one (75 mg, 0.33 mmol), Pd(dppf)Cl₂ (4.8 mg, 6.6 umol), Xantphos (7.6 mg, 13.2 umol), and Cs₂CO₃ (161 mg, 0.50 mmol) according to the procedure for compound 1. ¹H NMR (400 MHz, Methanol-d₄) δ 7.48 (d, J=8.3 Hz, 1H), 7.11-7.05 (m, 2H), 6.93-6.76 (m, 5H), 4.32 (s, 2H), 3.11 (s, 3H), 2.90 (s, 6H). Mass (m/z): 282.2 [M+H]⁺.

### Compound 228

### 5-([1,1′-biphenyl]-4-ylamino)-2-methylisoindolin-1-one

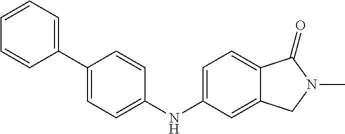 [SMILES: CN1Cc2cc(Nc3ccc(-c4ccccc4)cc3)ccc2C1=O]

The title compound 228 (33.6 mg) was prepared in a total yield of 32.0% as a yellow solid from 4-aminobiphenyl (73.0 mg, 0.43 mmol), 5-bromo-2-methylisoindolin-1-one (75 mg, 0.33 mmol), Pd(dppf)Cl₂ (4.8 mg, 6.6 umol), Xantphos (7.6 mg, 13.2 umol), and Cs₂CO₃ (161 mg, 0.50 mmol) according to the procedure for compound 1. ¹H NMR (400 MHz, Methanol-d₄) δ 7.62-7.54 (m, 5H), 7.44-7.37 (m, 2H), 7.31-7.21 (m, 4H), 7.12 (dd, J=8.3, 2.0 Hz, 1H), 4.40 (s, 2H), 3.15 (s, 3H). Mass (m/z): 315.3 [M+H]⁺.

### Compound 229

### 5-((2,3-dihydrobenzo[b][1,4]dioxin-6-yl)amino)-2-methylisoindolin-1-one

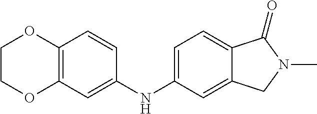 [SMILES: CN1Cc2cc(Nc3ccc4c(c3)OCCO4)ccc2C1=O]

The title compound 229 (47.4 mg) was prepared in a total yield of 32.0% as a yellow solid from 1,4-benzodioxan-6-amine (65.0 mg, 0.43 mmol), 5-bromo-2-methylisoindolin-1-one (75 mg, 0.33 mmol), Pd(dppf)Cl₂ (4.8 mg, 6.6 umol), Xantphos (7.6 mg, 13.2 umol), and Cs₂CO₃ (161 mg, 0.50 mmol) according to the procedure for compound 1. ¹H NMR (400 MHz, Methanol-d₄) δ 7.50 (dd, J=8.3, 0.6 Hz, 1H), 7.00-6.97 (m, 1H), 6.92 (dd, J=8.4, 2.0 Hz, 1H), 6.78 (d, J=8.4 Hz, 1H), 6.69-6.62 (m, 2H), 4.34 (s, 2H), 4.26-4.16 (m, 4H), 3.12 (s, 3H). Mass (m/z): 297.2 [M+H]⁺.

### Compound 230

### 5-((2-chloro-5-methylphenyl)amino)-2-methylisoindolin-1-one

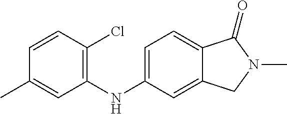

The title compound 230 (38.6 mg) was prepared in a total yield of 41.1% as a yellow solid from 2-Chloro-5-methylaniline (61.0 mg, 0.43 mmol), 5-bromo-2-methylisoindolin-1-one (75 mg, 0.33 mmol), Pd(dppf)Cl₂ (4.8 mg, 6.6 umol), Xantphos (7.6 mg, 13.2 umol), and Cs₂CO₃ (161 mg, 0.50 mmol) according to the procedure for compound 1. ¹H NMR (400 MHz, Methanol-d₄) δ 7.59-7.55 (m, 1H), 7.32-7.26 (m, 1H), 7.22 (s, 1H), 7.07-7.00 (m, 1H), 6.89-6.83 (m, 1H), 4.38 (s, 2H), 3.14 (s, 3H), 2.29 (s, 3H). Mass (m/z): 287.1 [M+H]⁺.

### Compound 231

### 5-((2-methoxy-5-methylphenyl)amino)-2-methylisoindolin-1-one

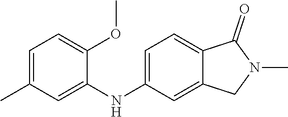

The title compound 231 (30.6 mg) was prepared in a total yield of 32.9% as a yellow solid from 2-Methoxy-5-methylaniline (59.0 mg, 0.43 mmol), 5-bromo-2-methylisoindolin-1-one (75 mg, 0.33 mmol), Pd(dppf)₂Cl₂ (4.8 mg, 6.6 umol), Xantphos (7.6 mg, 13.2 umol), and Cs₂CO₃ (161 mg, 0.50 mmol) according to the procedure for compound 1. ¹H NMR (400 MHz, Methanol-d₄) δ 7.52 (dd, J=7.9, 1.0 Hz, 1H), 7.13 (d, J=2.1 Hz, 1H), 7.05-6.98 (m, 2H), 6.89 (d, J=8.2 Hz, 1H), 6.83-6.79 (m, 1H), 4.34 (s, 2H), 3.81 (s, 3H), 3.12 (s, 3H), 2.26 (s, 3H). Mass (m/z): 283.2 [M+H]⁺.

### Compound 232

### 5-((4-(4-chlorophenoxy)phenyl)amino)-2-methylisoindolin-1-one

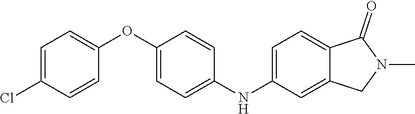 [SMILES: CN1Cc2cc(Nc3ccc(Oc4ccc(Cl)cc4)cc3)ccc2C1=O]

The title compound 232 (26.5 mg) was prepared in a total yield of 21.9% as a yellow solid from 4-(4-Chlorophenoxy)aniline (94.0 mg, 0.43 mmol), 5-bromo-2-methylisoindolin-1-one (75 mg, 0.33 mmol), Pd(dppf)₂Cl₂ (4.8 mg, 6.6 umol), Xantphos (7.6 mg, 13.2 umol), and Cs₂CO₃ (161 mg, 0.50 mmol) according to the procedure for compound 1. ¹H NMR (400 MHz, Methanol-d₄) δ 7.55 (d, J=8.4 Hz, 1H), 7.36-7.28 (m, 2H), 7.24-7.19 (m, 2H), 7.12-7.09 (m, 2H), 7.04-6.92 (m, 5H), 4.37 (s, 2H), 3.13 (s, 3H). Mass (m/z): 365.1 [M+H]⁺.

### Compound 233

### 5-(imidazo[1,2-a]pyridin-6-ylamino)-2-methylisoindolin-1-one

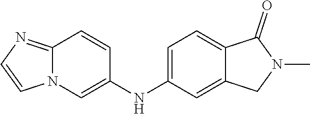 [SMILES: CN1Cc2cc(Nc3ccc4nccn4c3)ccc2C1=O]

The title compound 233 (31.7 mg) was prepared in a total yield of 34.2% as a yellow solid from imidazo[1,2-a]pyridin-6-amine (94.0 mg, 0.43 mmol), 5-bromo-2-methylisoindolin-1-one (75 mg, 0.33 mmol), Pd(dppf)₂Cl₂ (4.8 mg, 6.6 umol), Xantphos (7.6 mg, 13.2 umol), and Cs₂CO₃ (161 mg, 0.50 mmol) according to the procedure for compound 1. ¹H NMR (400 MHz, Methanol-d₄) δ 8.43-8.40 (m, 1H), 7.84 (d, J=1.4 Hz, 1H), 7.64-7.55 (m, 3H), 7.34 (dt, J=9.6, 1.9 Hz, 1H), 7.13-7.08 (m, 1H), 7.07-7.01 (m, 1H), 4.39 (s, 2H), 3.14 (s, 3H). Mass (m/z): 279.2 [M+H]⁺.

### Compound 234

### 5-((3-(dimethylamino)phenyl)amino)-2-methylisoindolin-1-one

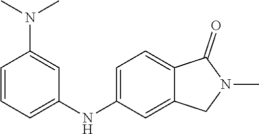 [SMILES: CN1Cc2cc(Nc3cccc(N(C)C)c3)ccc2C1=O]

The title compound 234 (34.6 mg) was prepared in a total yield of 36.9% as a yellow solid from N,N-dimethyl-m-phenylenediamine (58 mg, 0.43 mmol), 5-bromo-2-methylisoindolin-1-one (75 mg, 0.33 mmol), Pd(dppf)Cl₂ (4.8 mg, 6.6 umol), Xantphos (7.6 mg, 13.2 umol), and Cs₂CO₃ (163 mg, 0.50 mmol) according to the procedure for compound 1. ¹H NMR (400 MHz, Methanol-d₄) δ 7.54-7.50 (m, 1H), 7.15-7.09 (m, 2H), 7.05 (dd, J=8.5, 1.9 Hz, 1H), 6.60-6.56 (m, 1H), 6.55 (t, J=2.3 Hz, 1H), 6.47-6.41 (m, 1H), 4.36 (s, 2H), 3.13 (s, 3H), 2.91 (d, J=0.8 Hz, 6H). Mass (m/z): 282.2 [M+H]⁺.

### Compound 235

### 5-((6-(dimethylamino)pyridin-3-yl)amino)-2-methylisoindolin-1-one

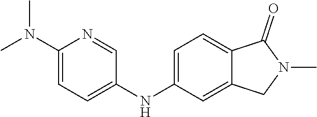

The title compound 235 (29.8 mg) was prepared in a total yield of 31.7% as a yellow solid from N2,N2-dimethylpyridine-2,5-diamine (59 mg, 0.43 mmol), 5-bromo-2-methylisoindolin-1-one (75 mg, 0.33 mmol), Pd(dppf)Cl₂ (4.8 mg, 6.6 umol), Xantphos (7.6 mg, 13.2 umol), and Cs₂CO₃ (163 mg, 0.50 mmol) according to the procedure for compound 1. ¹H NMR (400 MHz, Methanol-d₄) δ 7.94 (dt, J=2.7, 0.7 Hz, 1H), 7.51-7.42 (m, 2H), 6.84-6.79 (m, 2H), 6.71 (dt, J=9.0, 0.7 Hz, 1H), 4.32 (s, 2H), 3.11 (s, 3H), 3.07 (s, 6H). Mass (m/z): 283.2 [M+H]⁺.

### Compound 236

### 5-((4,6-dimethylpyridin-3-yl)amino)-2-methylisoindolin-1-one

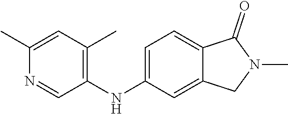

The title compound 236 (21.7 mg) was prepared in a total yield of 24.4% as a yellow solid from 4,6-dimethylpyridin-3-amine (52 mg, 0.43 mmol), 5-bromo-2-methylisoindolin-1-one (75 mg, 0.33 mmol), Pd(dppf)₂Cl₂ (4.8 mg, 6.6 umol), Xantphos (7.6 mg, 13.2 umol), and Cs₂CO₃ (163 mg, 0.50 mmol) according to the procedure for compound 1. ¹H NMR (400 MHz, Methanol-d₄) δ 7.94 (dt, J=2.7, 0.7 Hz, 1H), 7.51-7.42 (m, 2H), 6.84-6.79 (m, 2H), 6.71 (dt, J=9.0, 0.7 Hz, 1H), 4.32 (s, 2H), 3.11 (s, 3H), 3.07 (s, 6H). Mass (m/z): 268.2 [M+H]⁺.

### Compound 237

### 5-((4-(2-methoxyethoxy)phenyl)amino)-2-methylisoindolin-1-one

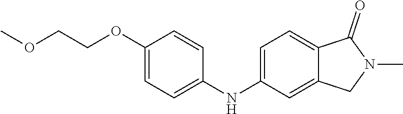

The title compound 237 (51.0 mg) was prepared in a total yield of 49.0% as a yellow solid from 4-(2-methoxyethoxy)aniline (72 mg, 0.43 mmol), 5-bromo-2-methylisoindolin-1-one (75 mg, 0.33 mmol), Pd(dppf)Cl₂ (4.8 mg, 6.6 umol), Xantphos (7.6 mg, 13.2 umol), and Cs₂CO₃ (163 mg, 0.50 mmol) according to the procedure for compound 1. ¹H NMR (400 MHz, Methanol-d₄) δ 7.49 (d, J=8.4 Hz, 1H), 7.15-7.06 (m, 2H), 6.98-6.85 (m, 5H), 4.32 (s, 2H), 4.14-4.04 (m, 2H), 3.76-3.72 (m, 2H), 3.42 (s, 3H), 3.11 (s, 3H). Mass (m/z): 313.3 [M+H]⁺.

### Compound 238

### 2-methyl-5-((4-(pyrrolidin-1-ylmethyl)phenyl)amino)isoindolin-1-one

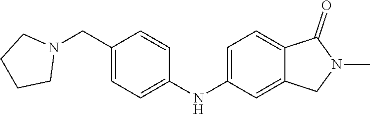 [SMILES: CN1Cc2cc(Nc3ccc(CN4CCCC4)cc3)ccc2C1=O]

The title compound 238 (24.6 mg) was prepared in a total yield of 23.0% as a yellow solid from 4-(Pyrrolidin-1-ylmethyl)aniline (76 mg, 0.43 mmol), 5-bromo-2-methylisoindolin-1-one (75 mg, 0.33 mmol), Pd(dppf)₂Cl₂ (4.8 mg, 6.6 umol), Xantphos (7.6 mg, 13.2 umol), and Cs₂CO₃ (163 mg, 0.50 mmol) according to the procedure for compound 1. ¹H NMR (400 MHz, Methanol-d₄) δ 7.59 (d, J=8.3 Hz, 1H), 7.46-7.41 (m, 2H), 7.27-7.23 (m, 3H), 7.15 (dd, J=8.4, 2.0 Hz, 1H), 4.40 (s, 2H), 4.30 (s, 2H), 3.37-3.31 (m, 4H), 3.14 (s, 3H), 2.15-2.01 (m, 4H). Mass (m/z): 322.3 [M+H]⁺.

### Compound 239

### 2-methyl-5-((4-(2-methylpiperidin-1-yl)phenyl)amino)isoindolin-1-one

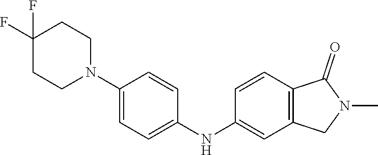 [SMILES: CN1Cc2cc(Nc3ccc(N4CCC(F)(F)CC4)cc3)ccc2C1=O]

The title compound 239 (22.7 mg) was prepared in a total yield of 28.7% as a white solid from 4-(4,4-difluoropiperidin-1-yl)aniline (61 mg, 0.288 mmol), 5-bromo-2-ethylisoindolin-1-one (50 mg, 0.221 mmol), Pd₂(dba)₃ (2 mg, 0.002 mmol), X-phos (6 mg, 0.01 mmol) and Cs₂CO₃ (109 mg, 0.332 mmol) and 1,4-dioxane (5 mL) according to the procedure for compound 1. ¹H NMR (400 MHz, Methanol-d₄) δ 7.63 (d, J=8.4 Hz, 1H), 7.49 (d, J=8.4 Hz, 2H), 7.33 (d, J=8.4 Hz, 2H), 7.28 (d, J=2.0 Hz, 1H), 7.19 (dd, J=8.4, 2.0 Hz, 1H), 4.43 (s, 2H), 3.77 (s, 1H), 3.61 (d, J=6.4 Hz, 2H), 3.16 (s, 3H), 2.09-1.91 (m, 3H), 1.80 (q, J=11.2, 10.0 Hz, 2H). Mass (m/z): 358.3 [M+H]⁺.

### Compound 240

### 2-methyl-5-((4-(2-methylpiperidin-1-yl)phenyl)amino)isoindolin-1-one

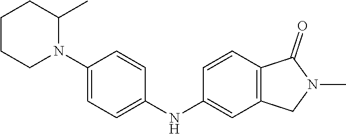 [SMILES: CC1CCCCN1c1ccc(Nc2ccc3c(c2)CN(C)C3=O)cc1]

The title compound 240 (46.4 mg) was prepared in a total yield of 62.7% as a white solid from 4-(2-methylpiperidin-1-yl)aniline (55 mg, 0.288 mmol), 5-bromo-2-ethylisoindolin-1-one (50 mg, 0.221 mmol), Pd₂(dba)₃ (2 mg, 0.002 mmol), X-phos (6 mg, 0.01 mmol), Cs₂CO₃ (109 mg, 0.332 mmol) and 1,4-dioxane (5 mL) according to the procedure for compound 1. ¹H NMR (400 MHz, Methanol-d₄) δ 7.63 (d, J=8.4 Hz, 1H), 7.49 (d, J=8.4 Hz, 2H), 7.33 (d, J=8.4 Hz, 2H), 7.28 (d, J=2.0 Hz, 1H), 7.19 (dd, J=8.4, 2.0 Hz, 1H), 4.43 (s, 2H), 3.77 (s, 1H), 3.61 (d, J=6.4 Hz, 2H), 3.16 (s, 3H), 2.16 (d, J=10.0 Hz, 1H), 2.09-1.91 (m, 3H), 1.80 (q, J=11.2, 10.0 Hz, 2H), 1.11 (d, J=6.4 Hz, 3H). Mass (m/z): 336.3 [M+H]⁺.

### Compound 241

### 2-methyl-5-((4-(3-methylpiperidin-1-yl)phenyl)amino)isoindolin-1-one

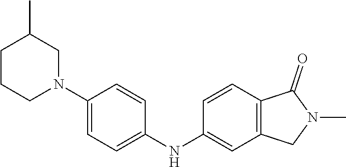 [SMILES: CC1CCCN(c2ccc(Nc3ccc4c(c3)CN(C)C4=O)cc2)C1]

The title compound 241 (32.5 mg) was prepared in a total yield of 43.9% as a white solid from 4-(3-methylpiperidin-1-yl)aniline (55 mg, 0.288 mmol), 5-bromo-2-ethylisoindolin-1-one (50 mg, 0.221 mmol), Pd₂(dba)₃ (2 mg, 0.002 mmol), X-phos (6 mg, 0.01 mmol), Cs₂CO₃ (109 mg, 0.332 mmol) and 1,4-dioxane (5 mL) according to the procedure for compound 1. ¹H NMR (400 MHz, Methanol-d₄) δ 7.63 (d, J=8.4 Hz, 1H), 7.49 (d, J=8.4 Hz, 2H), 7.33 (d, J=8.4 Hz, 2H), 7.28 (d, J=2.0 Hz, 1H), 7.19 (dd, J=8.4, 2.0 Hz, 1H), 4.43 (s, 2H), 3.77 (s, 1H), 3.61 (d, J=6.4 Hz, 2H), 3.16 (s, 3H), 2.16 (d, J=10.0 Hz, 1H), 2.09-1.91 (m, 3H), 1.80 (q, J=11.2, 10.0 Hz, 2H), 1.11 (d, J=6.4 Hz, 3H). Mass (m/z): 336.3 [M+H]⁺.

### Compound 242

### 2-methyl-5-((4-(3-(trifluoromethyl)piperidin-1-yl)phenyl)amino) isoindolin-1-one

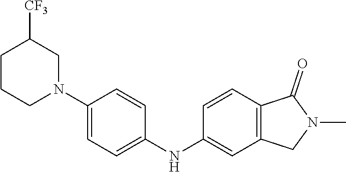 [SMILES: CN1Cc2cc(Nc3ccc(N4CCCC(C(F)(F)F)C4)cc3)ccc2C1=O]

The title compound 242 (16.9 mg) was prepared in a total yield of 19.7% as a white solid from 4-(3-(trifluoromethyl)piperidin-1-yl)aniline (70 mg, 0.288 mmol), 5-bromo-2-ethylisoindolin-1-one (50 mg, 0.221 mmol), Pd₂(dba)₃ (2 mg, 0.002 mmol), X-phos (6 mg, 0.01 mmol), Cs₂CO₃ (109 mg, 0.332 mmol) and 1,4-dioxane (5 mL) according to the procedure for compound 1. ¹H NMR (400 MHz, Methanol-d₄) δ 7.63 (d, J=8.4 Hz, 1H), 7.49 (d, J=8.4 Hz, 2H), 7.33 (d, J=8.4 Hz, 2H), 7.28 (d, J=2.0 Hz, 1H), 7.19 (dd, J=8.4, 2.0 Hz, 1H), 4.43 (s, 2H), 3.77 (s, 1H), 3.61 (d, J=6.4 Hz, 2H), 3.16 (s, 3H), 2.16 (d, J=10.0 Hz, 1H), 2.09-1.91 (m, 3H), 1.80 (q, J=11.2, 10.0 Hz, 2H). Mass (m/z): 390.3 [M+H]⁺.

### Compound 243

### 5-((3-methoxy-4-(4-(trifluoromethyl)piperidin-1-yl)phenyl)amino)-2-methylisoindolin-1-one

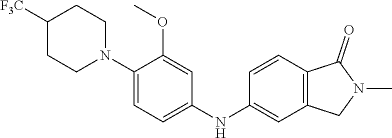 [SMILES: COc1cc(Nc2ccc3c(c2)CN(C)C3=O)ccc1N1CCC(C(F)(F)F)CC1]

The title compound 243 (49.0 mg) was prepared in a total yield of 52.9% as a white solid from 3-methoxy-4-(4-(trifluoromethyl)piperidin-1-yl)aniline (79 mg, 0.288 mmol), 5-bromo-2-ethylisoindolin-1-one (50 mg, 0.221 mmol), Pd₂(dba)₃ (2 mg, 0.002 mmol), X-phos (6 mg, 0.01 mmol), Cs₂CO₃ (109 mg, 0.332 mmol) and 1,4-dioxane (5 mL) according to the procedure for compound 1. ¹H NMR (400 MHz, Chloroform-d) δ 7.55 (d, J=8.0 Hz, 1H), 6.90-6.79 (m, 3H), 6.68 (dd, J=8.4, 2.4 Hz, 1H), 6.62 (d, J=2.4 Hz, 1H), 4.16 (s, 2H), 3.74 (s, 3H), 3.45 (d, J=11.2 Hz, 2H), 3.05 (s, 3H), 2.56-2.42 (m, 2H), 2.07 (tdt, J=11.6, 7.6, 3.6 Hz, 1H), 1.92-1.75 (m, 4H). Mass (m/z): 420.3 [M+H]⁺.

### Compound 244

### 5-((4-(2-azaspiro[3.3]heptan-2-yl)phenyl)amino)-2-methylisoindolin-1-one

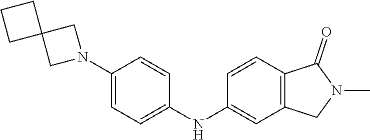 [SMILES: CN1Cc2cc(Nc3ccc(N4CC5(CCC5)C4)cc3)ccc2C1=O]

The title compound 244 (35.5 mg) was prepared in a total yield of 48.2% as a white solid from 4-(2-azaspiro[3.3]heptan-2-yl)aniline (54 mg, 0.288 mmol), 5-bromo-2-ethylisoindolin-1-one (50 mg, 0.221 mmol), Pd₂(dba)₃ (2 mg, 0.002 mmol), X-phos (6 mg, 0.01 mmol), Cs₂CO₃ (109 mg, 0.332 mmol) and 1,4-dioxane (5 mL) according to the procedure for compound 1. ¹H NMR (400 MHz, Chloroform-d) δ 7.57 (d, J=8.4 Hz, 1H), 7.03 (s, 2H), 6.83-6.68 (m, 2H), 6.43 (s, 2H), 5.90 (s, 1H), 4.18 (s, 2H), 3.46 (s, 1H), 3.10 (s, 3H), 2.20 (t, J=7.6 Hz, 4H), 1.93-1.83 (m, 2H). Mass (m/z): 334.3 [M+H]⁺.

### Compound 245

### 5-((4-((1R,4S)-2-azabicyclo[2.2.1]heptan-2-yl)phenyl)amino)-2-methylisoindolin-1-one

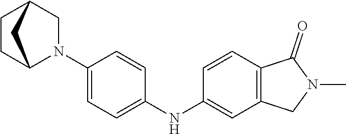 [SMILES: CN1Cc2cc(Nc3ccc(N4C[C@H]5CC[C@@H]4C5)cc3)ccc2C1=O]

The title compound 245 (32.9 mg) was prepared in a total yield of 44.6% as a white solid from 4-((1R,4S)-2-azabicyclo[2.2.1]heptan-2-yl)aniline (54 mg, 0.288 mmol), 5-bromo-2-ethylisoindolin-1-one (50 mg, 0.221 mmol), Pd₂(dba)₃ (2 mg, 0.002 mmol), X-phos (6 mg, 0.01 mmol), Cs₂CO₃ (109 mg, 0.332 mmol) and 1,4-dioxane (5 mL) according to the procedure for compound 1. ¹H NMR (400 MHz, Chloroform-d) δ 7.62-7.52 (m, 1H), 7.03 (s, 2H), 6.73 (s, 3H), 6.52 (s, 1H), 4.17 (s, 3H), 3.47 (s, 1H), 3.10 (s, 3H), 2.59 (s, 1H), 1.85-1.64 (m, 4H), 1.50 (s, 1H), 1.41-1.31 (m, 1H). Mass (m/z): 334.3 [M+H]⁺.

### Compound 246

### 5-((4-(2-azaspiro[3.4]octan-2-yl)phenyl)amino)-2-methylisoindolin-1-one

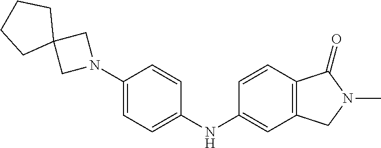 [SMILES: CN1Cc2cc(Nc3ccc(N4CC5(CCCC5)C4)cc3)ccc2C1=O]

The title compound 246 (35.5 mg) was prepared in a total yield of 43.7% as a purple solid from 4-(2-azaspiro[3.4]octan-2-yl)aniline (56 mg, 0.288 mmol), 5-bromo-2-ethylisoindolin-1-one (50 mg, 0.221 mmol), Pd₂(dba)₃ (2 mg, 0.002 mmol), X-phos (6 mg, 0.01 mmol), Cs₂CO₃ (109 mg, 0.332 mmol) and 1,4-dioxane (5 mL) according to the procedure for compound 1. ¹H NMR (400 MHz, Chloroform-d) δ 7.57 (d, J=8.4 Hz, 1H), 7.03 (s, 2H), 6.83-6.68 (m, 2H), 6.43 (s, 2H), 5.90 (s, 1H), 4.18 (s, 2H), 3.46 (s, 1H), 3.10 (s, 3H), 2.56-2.42 (m, 2H), 2.20 (t, J=7.6 Hz, 4H), 1.93-1.83 (m, 2H). Mass (m/z): 348.3 [M+H]⁺.

### Compound 247

### 5-((4-(4,4-dimethylpiperidin-1-yl)phenyl)amino)-2-(1-methylpiperidin-4-yl)isoindolin-1-one

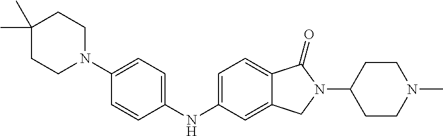

The title compound 247 (24.8 mg) was prepared in a yield of 35.45% as a pale yellow powder from 4-(4,4-dimethylpiperidin-1-yl)aniline (32 mg, 0.16 mmol) and 5-bromo-2-(1-methylpiperidin-4-yl)isoindolin-1-one (50 mg, 0.16 mmol) according to the procedure for compound 1. ¹H NMR (Methanol-d₄) δ 7.51 (d, J=8.4 Hz, 1H), 7.11 (s, 2H), 6.97 (d, J=29.5 Hz, 4H), 4.37 (s, 2H), 4.30 (ddt, J=11.9, 8.1, 4.2 Hz, 2H), 3.52 (dt, J=12.4, 2.5 Hz, 2H), 3.22-3.00 (m, 5H), 2.82 (s, 3H), 2.16 (qd, J=13.1, 4.0 Hz, 2H), 2.08-1.96 (m, 2H), 1.55 (t, J=5.7 Hz, 4H), 1.00 (s, 6H). Mass (m/z): 433.5 [M+H]⁺.

### Compound 248

### 2-((1-methyl-1H-imidazol-5-yl)methyl)-5-((4-(4-(trifluoromethyl) piperidin-1-yl)phenyl)amino)isoindolin-1-one

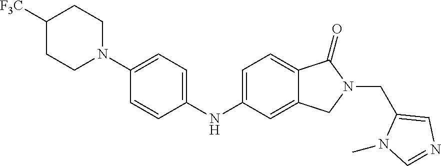 [SMILES: Cn1cncc1CN1Cc2cc(Nc3ccc(N4CCC(C(F)(F)F)CC4)cc3)ccc2C1=O]

The title compound 248 (24.5 mg) was prepared in a yield of 25.49% as a pale yellow powder from 4-(4-(trifluoromethyl)piperidin-1-yl)aniline (50 mg, 0.20 mmol) and 5-bromo-2-((1-methyl-1H-imidazol-5-yl) methyl)isoindolin-1-one (63 mg, 0.20 mmol) according to the procedure for compound 1. ¹H NMR (Methanol-d₄) δ 7.94 (s, 1H), 7.56 (d, J=8.4 Hz, 1H), 7.18-7.09 (m, 3H), 7.05-6.94 (m, 4H), 4.83 (s, 2H), 4.26 (s, 2H), 3.74 (s, 5H), 2.71 (t, J=12.0 Hz, 2H), 2.35 (dtd, J=12.3, 8.4, 3.8 Hz, 1H), 1.98 (d, J=13.1 Hz, 2H), 1.71 (qd, J=12.5, 4.1 Hz, 2H). Mass (m/z): 470.3 [M+H]⁺.

### Compound 249

### 5-((4-(4-methylpiperidin-1-yl)phenyl)amino)-2-(1-methylpiperidin-4-yl) isoindolin-1-one

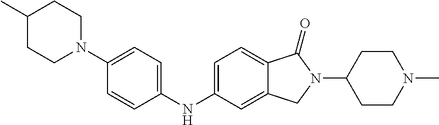

The title compound 249 (19.6 mg) was prepared in a yield of 28.96% as a pale yellow powder from 4-(4-methylpiperidin-1-yl)aniline (30 mg, 0.16 mmol) and 5-bromo-2-(1-methylpiperidin-4-yl)isoindolin-1-one (50 mg, 0.16 mmol) according to the procedure for compound 1. ¹H NMR (Methanol-d₄) δ: 7.51 (d, J=8.4 Hz, 1H), 7.14-7.08 (m, 2H), 7.05-6.97 (m, 3H), 6.94 (dd, J=8.5, 2.0 Hz, 1H), 4.37 (s, 2H), 4.28 (ddt, J=11.9, 8.3, 4.0 Hz, 1H), 3.55 (d, J=12.0 Hz, 2H), 3.45 (d, J=12.6 Hz, 2H), 2.96 (td, J=12.6, 2.9 Hz, 2H), 2.76 (s, 3H), 2.65 (s, 2H), 2.13 (qd, J=12.8, 3.7 Hz, 2H), 2.05-1.94 (m, 2H), 1.76 (dd, J=13.0, 3.0 Hz, 2H), 1.61-1.42 (m, 1H), 1.44-1.25 (m, 3H), 0.99 (d, J=6.4 Hz, 3H). Mass (m/z): 419.3 [M+H]⁺.

### Compound 250

### 1,2-dimethyl-6-((4-(4-methylpiperidin-1-yl)phenyl)amino)-1,2-dihydro-3H-indazol-3-one

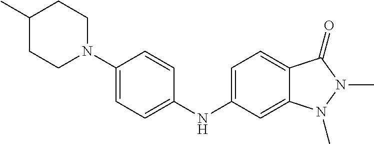

The title compound 250 (15.6 mg) was prepared in a total yield of 89.1% as a yellow solid from 4-(4-methylpiperidin-1-yl)aniline (12.4 mg, 0.065 mmol), 6-bromo-1,2-dimethyl-1,2-dihydro-3H-indazol-3-one (12.1 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (300 MHz, Chloroform-d) δ 7.46 (m, 1H), 7.15-6.45 (m, 6H), 3.83 (s, 3H), 3.63-3.45 (m, 2H), 3.05 (s, 3H), 2.76-2.48 (m, 2H), 1.76-1.28 (m, 5H), 0.90 (d, J=5.7 Hz, 3H). Mass (m/z): 351.2 [M+H]⁺.

### Compound 251

### 2-methyl-6-((4-(4-methylpiperidin-1-yl)phenyl)amino)-1,2-dihydro-3H-ind azol-3-one

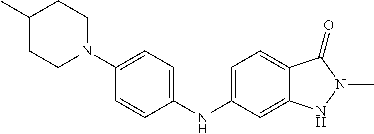

The title compound 251 (30.7 mg) was prepared in a total yield of 91.1% as a yellow solid from tert-butyl 2-methyl-6-((4-(4-methylpiperidin-1-yl)phenyl)amino)-3-oxo-2,3-dihydro-1H-indazole-1-carboxylate (43.6 mg, 0.1 mmol) and HCl in 1,4-dioxane according to the procedure for compound 1. ¹H NMR (400 MHz, Methanol-d₄) δ 7.71-7.59 (m, 3H), 7.41-7.33 (m, 2H), 7.01-6.94 (m, 2H), 3.72-3.61 (m, 7H), 2.13-2.01 (m, 2H), 1.91 (m, 1H), 1.83-1.72 (m, 2H), 1.10 (d, J=6.4 Hz, 3H). Mass (m/z): 337.2 [M+H]⁺.

### Compound 252

### 2-ethyl-6-((4-(4-(trifluoromethyl)piperidin-1-yl)phenyl)amino)-1,2-dihydro-3H-indazol-3-one

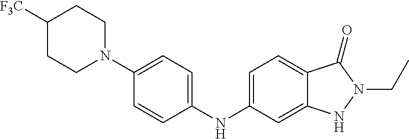 [SMILES: CCn1[nH]c2cc(Nc3ccc(N4CCC(C(F)(F)F)CC4)cc3)ccc2c1=O]

The title compound 252 (18.3 mg) was prepared in a total yield of 90.6% as a yellow solid from tert-butyl 2-ethyl-3-oxo-6-((4-(4-(trifluoromethyl)piperidin-1-yl)phenyl)amino)-2,3-dihydro-1H-indazole-1-carboxylate (25.2 mg, 0.05 mmol) and HCl in 1,4-dioxane according to the procedure for compound 1. ¹H NMR (400 MHz, Methanol-d₄) δ 7.68-7.60 (m, 3H), 7.38-7.33 (m, 2H), 7.00-6.90 (m, 2H), 3.98 (q, J=7.2 Hz, 2H), 3.85-3.65 (m, 4H), 2.80 (m, 1H), 2.34-2.26 (m, 2H), 2.23-2.11 (m, 2H), 1.37 (t, J=7.2 Hz, 3H). Mass (m/z): 405.2 [M+H]⁺.

### Compound 253

### 1-(4-chloro-6-((4-(4-methylpiperidin-1-yl)phenyl)amino)isoindolin-2-yl)-2,2,2-trifluoroethan-1-one

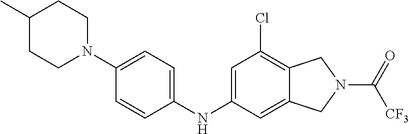 [SMILES: CC1CCN(c2ccc(Nc3cc(Cl)c4c(c3)CN(C(=O)C(F)(F)F)C4)cc2)CC1]

The title compound 253 (19.1 mg) was prepared in a total yield of 87.4% as a red solid from 4-(4-methylpiperidin-1-yl)aniline (12.4 mg, 0.065 mmol), 1-(6-bromo-4-chloroisoindolin-2-yl)-2,2,2-trifluoroethan-1-one (16.4 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.33-6.78 (m, 6H), 4.90 (s, 2H), 4.78 (s, 2H), 3.74-3.65 (m, 2H), 2.79-2.67 (m, 2H), 1.91-1.70 (m, 2H), 1.55 (m, 1H), 1.47-1.36 (m, 2H), 1.02 (d, J=6.4 Hz, 3H). Mass (m/z): 438.1 [M+H]⁺.

### Compound 254

### 5-((4-(2,5-dimethylpyrrolidin-1-yl)phenyl)amino)-1,3-dimethyl-1,3-dihydro-2H-benzo[d]imidazol-2-one

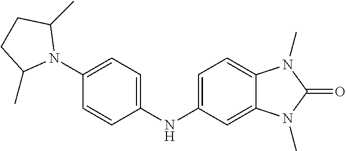 [SMILES: CC1CCC(C)N1c1ccc(Nc2ccc3c(c2)n(C)c(=O)n3C)cc1]

The title compound 254 (13.4 mg) was prepared in a total yield of 76.4% as a white solid from 4-(2,5-dimethylpyrrolidin-1-yl)aniline (12.4 mg, 0.065 mmol), 5-bromo-2-methylisoindoline-1,3-dione (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.61-6.57 (m, 7H), 3.55-3.51 (m, 2H), 3.45 (s, 3H), 3.37 (s, 3H), 2.16-1.67 (m, 4H), 1.15 (d, J=6.4 Hz, 6H). Mass (m/z): 351.2 [M+H]⁺.

### Compound 255

### 2,2,2-trifluoro-1-(6-((4-(4-methylpiperidin-1-yl)phenyl)amino)-4-nitroisoindolin-2-yl)ethan-1-one

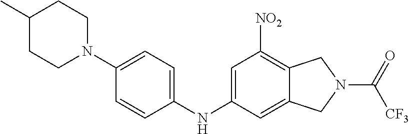 [SMILES: CC1CCN(c2ccc(Nc3cc4c(c([N+](=O)[O-])c3)CN(C(=O)C(F)(F)F)C4)cc2)CC1]

The title compound 255 (18.7 mg) was prepared in a total yield of 83.5% as a red solid from 4-(4-methylpiperidin-1-yl)aniline (12.4 mg, 0.065 mmol), 1-(6-bromo-4-nitroisoindolin-2-yl)-2,2,2-trifluoroethan-1-one (16.9 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.65-6.81 (m, 6H), 4.94 (s, 2H), 4.79 (s, 2H), 3.51-3.40 (m, 2H), 2.72-2.66 (m, 2H), 1.79-1.68 (m, 2H), 1.48 (m, 1H), 1.43-1.31 (m, 2H), 0.99 (d, J=6.4 Hz, 3H). Mass (m/z): 449.3 [M+H]⁺.

### Compound 256

### 6-((4-(2,5-dimethylpyrrolidin-1-yl)phenyl)amino)-3-methylbenzo[d]oxazol-2(3H)-one

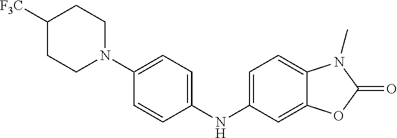 [SMILES: Cn1c(=O)oc2cc(Nc3ccc(N4CCC(C(F)(F)F)CC4)cc3)ccc21]

The title compound 256 (11.3 mg) was prepared in a total yield of 66.7% as a white solid from 4-(4-(trifluoromethyl)piperidin-1-yl)aniline (15.9 mg, 0.065 mmol), 6-bromo-3-methylbenzo[d]oxazol-2(3H)-one (11.4 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.21-6.78 (m, 7H), 3.68-3.57 (m, 2H), 3.35 (s, 3H), 2.75-2.57 (m, 2H), 2.27 (m, 1H), 2.03-1.93 (m, 2H), 1.83-1.67 (m, 2H). Mass (m/z): 392.1 [M+H]⁺.

### Compound 257

### 2-methyl-6-((4-(piperidin-1-yl)phenyl)amino)-1,2-dihydro-3H-indazol-3-one

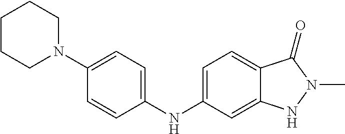 [SMILES: Cn1[nH]c2cc(Nc3ccc(N4CCCCC4)cc3)ccc2c1=O]

The title compound 257 (28.7 mg) was prepared in a total yield of 88.9% as a white solid from tert-butyl 2-methyl-3-oxo-6-((4-(piperidin-1-yl) phenyl)amino)-2,3-dihydro-1H-indazole-1-carboxylate (42.2 mg, 0.1 mmol) and HCl according to the procedure for compound 1. ¹H NMR (400 MHz, Methanol-d₄) δ 7.61-6.81 (m, 7H), 3.26 (s, 3H), 2.96-2.85 (m, 4H), 1.93-1.74 (m, 4H), 1.68-1.57 (m, 2H). Mass (m/z): 323.2 [M+H]⁺.

### Compound 258

### 2-(tetrahydro-2H-pyran-4-yl)-5-((4-(4-(trifluoromethyl)piperidin-1-yl) phenyl)amino)isoindolin-1-one

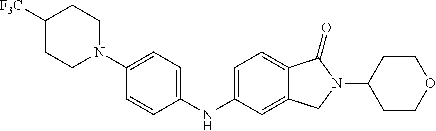 [SMILES: O=C1c2ccc(Nc3ccc(N4CCC(C(F)(F)F)CC4)cc3)cc2CN1C1CCOCC1]

The title compound 258 (10.4 mg) was prepared in a yield of 11.06% as a pale yellow powder from 4-(4-(trifluoromethyl)piperidin-1-yl)aniline (50 mg, 0.20 mmol) and 5-bromo-2-(tetrahydro-2H-pyran-4-yl)isoindolin-1-one (60 mg, 0.20 mmol) according to the procedure for compound 1. ¹H NMR (DMSO-d₆) δ 8.30 (s, 1H), 7.41 (d, J=8.4 Hz, 1H), 7.05 (d, J=8.9 Hz, 2H), 6.95 (dd, J=9.0, 2.5 Hz, 3H), 6.89 (dd, J=8.3, 2.0 Hz, 1H), 4.30 (s, 2H), 4.24-4.12 (m, 2H), 3.95-3.87 (m, 2H), 3.68 (d, J=12.3 Hz, 2H), 3.42 (t, J=11.6 Hz, 2H), 2.62 (s, 2H), 1.88 (d, J=13.2 Hz, 2H), 1.82-1.68 (m, 2H), 1.64-1.51 (m, 4H). Mass (m/z): 460.3 [M+H]⁺.

### Compound 259

### 6-((4-(2,5-dimethylpyrrolidin-1-yl)phenyl)amino)-2-methyl-1,2-dihydro-3H-indazol-3-one

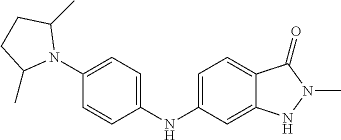 [SMILES: CC1CCC(C)N1c1ccc(Nc2ccc3c(=O)n(C)[nH]c3c2)cc1]

The title compound 259 (30.7 mg) was prepared in a total yield of 91.1% as a white solid from tert-butyl 6-((4-(2,5-dimethylpyrrolidin-1-yl) phenyl)amino)-2-methyl-3-oxo-2,3-dihydro-1H-indazole-1-carboxylate (43.6 mg, 0.1 mmol) and HCl according to the procedure for compound 1. ¹H NMR (400 MHz, Methanol-d₄) δ 7.77-7.18 (m, 5H), 6.89-6.78 (m, 2H), 4.04-3.83 (m, 2H), 3.37 (s, 3H), 2.37-2.31 (m, 2H), 1.98-1.89 (m, 2H), 1.25 (d, J=6.4 Hz, 6H). Mass (m/z): 337.2 [M+H]⁺.

### Compound 260

### 5-((4-(ethyl(pentyl)amino)phenyl)amino)-1,3-dimethyl-1,3-dihydro-2H-benzo[d]imidazol-2-one

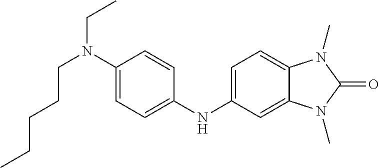 [SMILES: CCCCCN(CC)c1ccc(Nc2ccc3c(c2)n(C)c(=O)n3C)cc1]

The title compound 260 (11.3 mg) was prepared in a total yield of 12.3% as a yellow solid from N¹-ethyl-N¹-pentylbenzene-1,4-diamine (67 mg, 0.33 mmol), 5-bromo-1,3-dimethyl-2,3-dihydro-1H-1,3-benzodiazol-2-one (60 mg, 0.25 mmol), Pd(dppf)Cl₂ (3.7 mg, 5 umol), Xantphos (5.8 mg, 10.0 umol), and Cs₂CO₃ (122 mg, 0.38 mmol) according to the procedure for compound 1. ¹H NMR (400 MHz, DMSO-d₆) δ 7.72-6.81 (m, 8H), 3.65-3.29 (m, 10H), 1.49-1.15 (m, 4H), 1.07-0.99 (m, 2H), 0.93-0.77 (m, 6H). Mass (m/z): 367.3 [M+H]⁺.

### Compound 261

### 5-((4-(tert-butyl)phenyl)amino)-1,3-dimethyl-1,3-dihydro-2H-benzo[d]imidazol-2-one

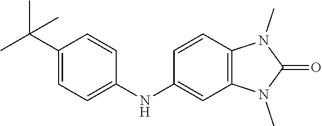 [SMILES: Cn1c(=O)n(C)c2cc(Nc3ccc(C(C)(C)C)cc3)ccc21]

The title compound 261 (39.9 mg) was prepared in a total yield of 31.0% as a white solid from 4-(tert-butyl)aniline (80 mg, 0.542 mmol), 5-bromo-1,3-dimethyl-1,3-dihydro-2H-benzo[d]imidazol-2-one (100 mg, 0.417 mmol), Pd₂(dba)₃ (4 mg, 0.004 mmol), X-phos (12 mg, 0.02 mmol), Cs₂CO₃ (205 mg, 0.625 mmol) and 1,4-dioxane (5 mL) according to the procedure for compound 1. ¹H NMR (400 MHz, DMSO-d₆) δ 7.92 (s, 1H), 7.20 (d, J=8.4 Hz, 2H), 7.00 (d, J=8.4 Hz, 1H), 6.94 (d, J=8.4 Hz, 2H), 6.87 (d, J=2.0 Hz, 1H), 6.78 (dd, J=8.4, 2.0 Hz, 1H), 3.28 (d, J=7.6 Hz, 6H), 1.24 (s, 9H). Mass (m/z): 310.3 [M+H]⁺.

### Compound 262

### 5-((4-(ethyl(3-methoxypropyl)amino)phenyl)amino)-1,3-dimethyl-1,3-dihydro-2H-benzo[d]imidazol-2-one

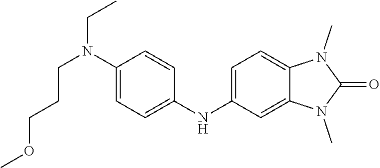 [SMILES: CCN(CCCOC)c1ccc(Nc2ccc3c(c2)n(C)c(=O)n3C)cc1]

The title compound 262 (8.3 mg) was prepared in a total yield of 6.8% as a gray powder from N¹-ethyl-N¹-(3-methoxypropyl)benzene-1,4-diamine (89 mg, 0.43 mmol), 5-bromo-1,3-dimethyl-2,3-dihydro-1H-1,3-benzodiazol-2-one (80 mg, 0.33 mmol), Pd(dppf)Cl₂ (4.8 mg, 6.6 umol), Xantphos (7.6 mg, 13.2 umol), and Cs₂CO₃ (163 mg, 0.50 mmol) according to the procedure for compound 1. ¹H NMR (400 MHz, DMSO-d₆) δ 7.75-6.69 (m, 8H), 3.65-3.29 (m, 15H), 1.10-1.02 (m, 2H), 0.93-0.77 (m, 3H). Mass (m/z): 369.2 [M+H]⁺.

### Compound 263

### 5-((2-fluoro-3-methyl-4-(4-methylpiperidin-1-yl)phenyl)amino)-1,3-dimethyl-1,3-dihydro-2H-benzo[d]imidazol-2-one

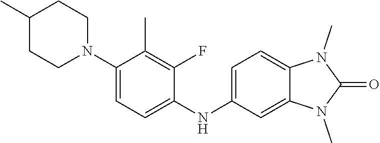 [SMILES: Cc1c(N2CCC(C)CC2)ccc(Nc2ccc3c(c2)n(C)c(=O)n3C)c1F]

The title compound 263 (21.4 mg) was prepared in a total yield of 16.9% as a pink powder from 2-fluoro-3-methyl-4-(4-methylpiperidin-1-yl)aniline (95 mg, 0.43 mmol), 5-bromo-1,3-dimethyl-2,3-dihydro-1H-1,3-benzodiazol-2-one (80 mg, 0.33 mmol), Pd(dppf)Cl₂ (4.8 mg, 6.6 umol), Xantphos (7.6 mg, 13.2 umol), and Cs₂CO₃ (163 mg, 0.50 mmol) according to the procedure for compound 1. ¹H NMR (301 MHz, Methanol-d₄) δ 7.27-6.91 (m, 5H), 3.69-3.49 (m, 4H), 3.42 (s, 3H), 3.39 (s, 3H), 2.43 (s, 3H), 2.10-1.97 (m, 2H), 1.89-1.71 (m, 3H), 1.09 (d, J=5.9 Hz, 3H). Mass (m/z): 383.3 [M+H]⁺.

### Compound 264

### 5-((5-fluoro-2-methyl-4-(4-methylpiperidin-1-yl)phenyl)amino)-1,3-dimethyl-1,3-dihydro-2H-benzo[d]imidazol-2-one

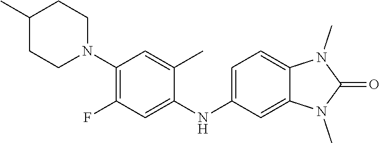 [SMILES: Cc1cc(N2CCC(C)CC2)c(F)cc1Nc1ccc2c(c1)n(C)c(=O)n2C]

The title compound 264 (12.1 mg) was prepared in a total yield of 10.0% as a light pink powder from 5-fluoro-2-methyl-4-(4-methylpiperidin-1-yl)aniline (95 mg, 0.43 mmol), 5-bromo-1,3-dimethyl-2,3-dihydro-1H-1,3-benzodiazol-2-one (80 mg, 0.33 mmol), Pd(dppf)Cl₂ (4.8 mg, 6.6 umol), Xantphos (7.6 mg, 13.2 umol), and Cs₂CO₃ (163 mg, 0.50 mmol) according to the procedure for compound 1. ¹H NMR (300 MHz, Methanol-d₄) δ 6.99 (d, J=8.2 Hz, 1H), 6.89 (d, J=9.6 Hz, 1H), 6.79-6.67 (m, 3H), 3.39 (s, 3H), 3.35 (s, 3H), 3.27 (s, 2H), 2.65 (t, J=11.3 Hz, 2H), 2.19 (s, 3H), 1.79-1.71 (m, 2H), 1.50-1.37 (m, 3H), 1.00 (d, J=6.0 Hz, 3H). Mass (m/z): 383.3 [M+H]⁺.

### Compound 265

### 5-((5-chloro-2-methyl-4-(4-methylpiperidin-1-yl)phenyl)amino)-1,3-dimethyl-1,3-dihydro-2H-benzo[d]imidazol-2-one

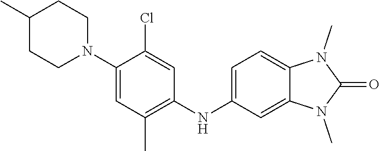 [SMILES: Cc1cc(N2CCC(C)CC2)c(Cl)cc1Nc1ccc2c(c1)n(C)c(=O)n2C]

The title compound 265 (28.9 mg) was prepared in a total yield of 21.8% as a white powder from 5-chloro-2-methyl-4-(4-methylpiperidin-1-yl) aniline (102 mg, 0.43 mmol), 5-bromo-1,3-dimethyl-2,3-dihydro-1H-1,3-benzodiazol-2-one (80 mg, 0.33 mmol), Pd(dppf)Cl₂ (4.8 mg, 6.6 umol), Xantphos (7.6 mg, 13.2 umol), and Cs₂CO₃ (163 mg, 0.50 mmol) according to the procedure for compound 1. ¹H NMR (300 MHz, Methanol-d₄) δ 7.14-6.94 (m, 3H), 6.72-6.65 (m, 2H), 3.39 (s, 3H), 3.34 (s, 3H), 3.24 (d, J=11.4 Hz, 2H), 2.62 (t, J=11.4 Hz, 2H), 2.20 (s, 3H), 1.77-1.70 (m, 2H), 1.49-1.37 (m, 3H), 1.01 (d, J=5.9 Hz, 4H). Mass (m/z): 399.3 [M+H]⁺.

### Compound 266

### 1,3-dimethyl-5-((1-methyl-1H-indol-5-yl)amino)-1,3-dihydro-2H-benzo[d]imidazol-2-one

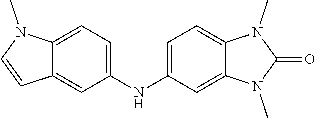 [SMILES: Cn1ccc2cc(Nc3ccc4c(c3)n(C)c(=O)n4C)ccc21]

The title compound 266 (28.9 mg) was prepared in a total yield of 21.8% as a white powder from 1-methyl-1H-indol-5-amine (63 mg, 0.43 mmol), 5-bromo-1,3-dimethyl-2,3-dihydro-1H-1,3-benzodiazol-2-one (80 mg, 0.33 mmol), Pd(dppf)Cl₂ (4.8 mg, 6.6 umol), Xantphos (7.6 mg, 13.2 umol), and Cs₂CO₃ (163 mg, 0.50 mmol) according to the procedure for compound 1. ¹H NMR (400 MHz, Methanol-d₄) δ 7.31 (d, J=7.0 Hz, 1H), 7.30 (s, 1H), 7.12 (d, J=3.0 Hz, 1H), 7.02 (dd, J=8.6, 2.2 Hz, 1H), 6.97 (d, J=8.4 Hz, 1H), 6.80 (dd, J=8.3, 2.1 Hz, 1H), 6.76 (d, J=2.1 Hz, 1H), 6.33 (dd, J=3.0, 0.8 Hz, 1H), 3.80 (s, 3H), 3.39 (s, 3H), 3.34 (s, 3H). Mass (m/z): 307.3 [M+H]⁺.

### Compound 267

### 7-chloro-2-methyl-N-(4-(4-methylpiperidin-1-yl)phenyl)isoindolin-5-amine

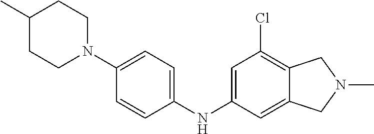

The title compound 267 (12.6 mg) was prepared in a total yield of 70.8% as a dim gray powder from 4-(4-methylpiperidin-1-yl)aniline (12.4 mg, 0.065 mmol), 6-bromo-4-chloro-2-methylisoindoline (12.3 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.29 (s, 1H), 7.14-6.86 (m, 5H), 4.30 (s, 2H), 4.24 (s, 2H), 3.65-3.53 (m, 2H), 2.88 (s, 3H), 2.73-2.59 (m, 2H), 1.85-1.73 (m, 2H), 1.52 (m, 1H), 1.44-1.27 (m, 2H), 1.00 (d, J=6.4 Hz, 3H). Mass (m/z): 356.2 [M+H]⁺.

### Compound 268

### 5-((4-(diethylamino)phenyl)amino)-1,3-dimethyl-1,3-dihydro-2H-benzo[d]imidazol-2-one

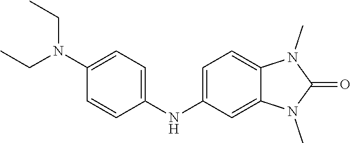 [SMILES: CCN(CC)c1ccc(Nc2ccc3c(c2)n(C)c(=O)n3C)cc1]

The title compound 268 (14.1 mg) was prepared in a yield of 14.28% as a pale yellow powder from N¹,N¹-diethylbenzene-1,4-diamine (50 mg, 0.30 mmol) and 5-bromo-1,3-dimethyl-1,3-dihydro-2H-benzo[d]imidazol-2-one (73 mg, 0.30 mmol) according to the procedure for compound 1. ¹H NMR (DMSO-d₆) δ 8.41 (s, 1H), 7.61 (s, 1H), 7.40 (s, 1H), 7.06 (s, 2H), 6.97 (s, 1H), 6.52 (s, 1H), 3.53 (m, 4H), 1.24 (s, 6H), 1.17 (d, J=12.0 Hz, 6H). Mass (m/z): 325.6 [M+H]⁺.

### Compound 269

### 5-((3-methoxy-4-(4-(trifluoromethyl)piperidin-1-yl)phenyl)amino)-1,3-dimethyl-1,3-dihydro-2H-benzo[d]imidazol-2-one

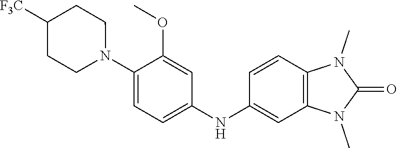 [SMILES: COc1cc(Nc2ccc3c(c2)n(C)c(=O)n3C)ccc1N1CCC(C(F)(F)F)CC1]

The title compound 269 (27.1 mg) was prepared in a total yield of 15.0% as a white solid from 3-methoxy-4-(4-(trifluoromethyl)piperidin-1-yl)aniline (148 mg, 0.542 mmol), 5-bromo-1,3-dimethyl-1,3-dihydro-2H-benzo[d]imidazol-2-one (100 mg, 0.417 mmol), Pd₂(dba)₃ (4 mg, 0.004 mmol), X-phos (12 mg, 0.02 mmol), Cs₂CO₃ (205 mg, 0.625 mmol) and 1,4-dioxane (5 mL) according to the procedure for compound 1. ¹H NMR (400 MHz, Chloroform-d) δ 7.55 (d, J=8.0 Hz, 1H), 6.90-6.79 (m, 3H), 6.68 (dd, J=8.4, 2.4 Hz, 1H), 6.62 (d, J=2.4 Hz, 1H), 3.89 (d, J=7.6 Hz, 6H), 3.74 (s, 3H), 3.45 (d, J=11.2 Hz, 2H), 2.56-2.42 (m, 2H), 2.07 (tdt, J=11.6, 7.6, 3.6 Hz, 1H), 1.92-1.75 (m, 4H). Mass (m/z): 435.3 [M+H]⁺.

### Compound 270

### 1-methyl-N-(4-(4-methylpiperidin-1-yl)phenyl)-1H-benzo[d]imidazol-6-amine

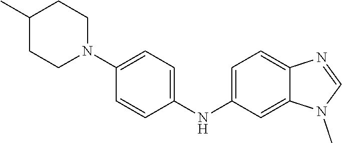 [SMILES: CC1CCN(c2ccc(Nc3ccc4ncn(C)c4c3)cc2)CC1]

The title compound 270 (19.4 mg) was prepared in a total yield of 12.1% as a gray solid from 4-(4-methylpiperidin-1-yl)aniline (124 mg, 0.65 mmol), 6-bromo-1-methyl-1H-1,3-benzodiazole (105 mg, 0.50 mmol), Pd(dppf)Cl₂ (7.3 mg, 10 umol), Xantphos (11.6 mg, 20 umol), and Cs₂CO₃ (245 mg, 0.75 mmol) according to the procedure for compound 1. ¹H NMR (400 MHz, DMSO-d₆) δ 8.21 (s, 1H), 7.89 (s, 1H), 7.58 (d, J=8.8 Hz, 1H), 7.08-7.00 (m, 3H), 6.98-6.82 (m, 3H), 3.77 (s, 3H), 3.55-3.48 (m, 2H), 2.61-2.52 (m, 2H), 1.73-1.65 (m, 2H), 1.53-1.38 (m, 1H), 1.31-1.20 (m, 2H), 0.95 (d, J=6.5 Hz, 3H). Mass (m/z): 321.3 [M+H]⁺.

### Compound 271

### 1,3-dimethyl-5-((4-(pyrrolidin-1-yl)phenyl)amino)-1,3-dihydro-2H-benzo[d]imidazol-2-one

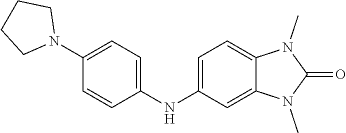 [SMILES: Cn1c(=O)n(C)c2cc(Nc3ccc(N4CCCC4)cc3)ccc21]

The title compound 271 (19.4 mg) was prepared in a total yield of 12.1% as a gray solid from 4-(pyrrolidin-1-yl)aniline (73 mg, 0.45 mmol), 5-bromo-1,3-dimethyl-2,3-dihydro-1H-1,3-benzodiazol-2-one (80 mg, 0.33 mmol), Pd(dppf)₂Cl₂ (4.8 mg, 6.6 umol), Xantphos (7.6 mg, 13.2 umol), and Cs₂CO₃ (163 mg, 0.50 mmol) according to the procedure for compound 1. ¹H NMR (400 MHz, Methanol-d₄) δ 7.86-6.01 (m, 8H), 3.56-3.33 (m, 8H), 3.30-3.14 (m, 2H), 2.11-1.89 (m, 4H). Mass (m/z): 323.2 [M+H]⁺.

### Compound 272

### 5-((2-fluoro-6-methoxy-4-(4-methylpiperidin-1-yl)phenyl)amino)-1,3-dimethyl-1,3-dihydro-2H-benzo[d]imidazol-2-one

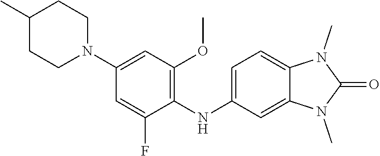 [SMILES: COc1cc(N2CCC(C)CC2)cc(F)c1Nc1ccc2c(c1)n(C)c(=O)n2C]

The title compound 272 (2.4 mg) was prepared in a total yield of 2.1% as a gray solid from 2-fluoro-6-methoxy-4-(4-methylpiperidin-1-yl)aniline (90 mg, 0.38 mmol), 5-bromo-1,3-dimethyl-2,3-dihydro-1H-1,3-benzodiazol-2-one (70 mg, 0.29 mmol), Pd(dppf)Cl₂ (4.2 mg, 5.8 umol), Xantphos (6.7 mg, 11.6 umol), and Cs₂CO₃ (142 mg, 0.44 mmol) according to the procedure for compound 1. ¹H NMR (400 MHz, Methanol-d₄) δ 7.18-6.84 (m, 3H), 6.73-6.45 (m, 2H), 4.00-3.89 (m, 2H), 3.49-3.36 (m, 5H), 3.35 (s, 3H), 3.34 (s, 3H), 2.13-1.98 (m, 2H), 1.88-1.76 (m, 1H), 1.68-1.57 (m, 2H), 1.10 (d, J=6.3 Hz, 3H). Mass (m/z): 399.3 [M+H]⁺.

### Compound 273

### 3-methyl-6-((4-(4-methylpiperidin-1-yl)phenyl)amino) benzo[d]oxazol-2(3H)-one

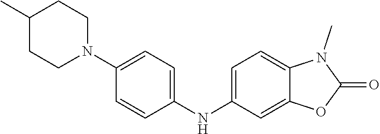 [SMILES: CC1CCN(c2ccc(Nc3ccc4c(c3)oc(=O)n4C)cc2)CC1]

The title compound 273 (12.6 mg) was prepared in a total yield of 70.8% as a dim gray powder from 4-(4-methylpiperidin-1-yl)aniline (12.4 mg, 0.065 mmol), 6-bromo-3-methylbenzo[d]oxazol-2(3H)-one (11.4 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.58-6.64 (m, 7H), 3.53-3.37 (m, 2H), 3.26 (s, 3H), 2.77-2.63 (m, 2H), 1.87-1.72 (m, 2H), 1.55 (m, 1H), 1.46-1.29 (m, 2H), 1.01 (d, J=6.4 Hz, 3H). Mass (m/z): 338.3 [M+H]⁺.

### Compound 274

### 5-((3,5-difluoro-4-(4-methylpiperidin-1-yl)phenyl)amino)-1,3-dimethyl-1,3-dihydro-2H-benzo[d]imidazol-2-one

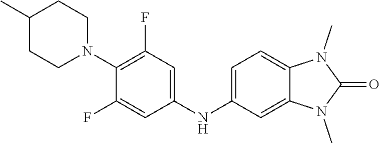 [SMILES: CC1CCN(c2c(F)cc(Nc3ccc4c(c3)n(C)c(=O)n4C)cc2F)CC1]

The title compound 274 (15.2 mg) was prepared in a total yield of 78.6% as a violet solid from 3,5-difluoro-4-(4-methylpiperidin-1-yl)aniline (14.7 mg, 0.065 mmol), 5-bromo-2-methylisoindoline-1,3-dione (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.01-6.65 (m, 5H), 3.65-3.56 (m, 2H), 3.39 (s, 3H), 3.35 (s, 3H), 2.76-2.61 (m, 2H), 1.81-1.73 (m, 2H), 1.54 (m, 1H), 1.48-1.35 (m, 3H), 1.01 (d, J=6.4 Hz, 3H). Mass (m/z): 387.2 [M+H]⁺.

### Compound 275

### 5-((3-fluoro-2-methyl-4-(4-methylpiperidin-1-yl)phenyl)amino)-1,3-dimethyl-1,3-dihydro-2H-benzo[d]imidazol-2-one

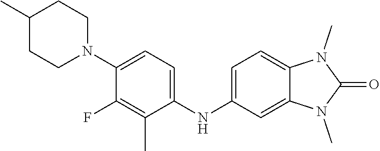 [SMILES: Cc1c(Nc2ccc3c(c2)n(C)c(=O)n3C)ccc(N2CCC(C)CC2)c1F]

The title compound 275 (15.2 mg) was prepared in a total yield of 78.6% as a violet solid from 3-fluoro-2-methyl-4-(4-methylpiperidin-1-yl)aniline (14.4 mg, 0.065 mmol), 5-bromo-2-methylisoindoline-1,3-dione (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 6.97 (d, J=8.4 Hz, 1H), 6.87-6.82 (m, 2H), 6.69-6.64 (m, 2H), 3.67-3.55 (m, 2H), 3.38 (s, 3H), 3.33 (s, 3H), 2.70-2.59 (m, 2H), 2.14 (s, 3H), 1.80-1.72 (m, 2H), 1.58-1.35 (m, 3H), 1.01 (d, J=6.4 Hz, 3H). Mass (m/z): 383.2 [M+H]⁺.

### Compound 276

### 1,3-dimethyl-5-((6-(4-methylpiperidin-1-yl)pyridin-3-yl)amino)-1,3-dihydro-2H-benzo[d]imidazol-2-one

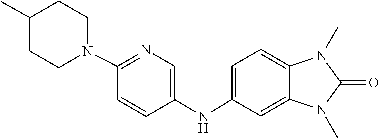 [SMILES: CC1CCN(c2ccc(Nc3ccc4c(c3)n(C)c(=O)n4C)cn2)CC1]

The title compound 276 (14.3 mg) was prepared in a total yield of 81.3% as a white solid from 6-(4-methylpiperidin-1-yl)pyridin-3-amine (12.4 mg, 0.065 mmol), 5-bromo-2-methylisoindoline-1,3-dione (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.51 (d, J=2.0 Hz, 1H), 7.34 (d, J=8.4 Hz, 1H), 6.94-6.58 (m, 4H), 4.14-4.05 (m, 2H), 3.31 (s, 3H), 3.26 (s, 3H), 2.72-2.64 (m, 2H), 1.71-1.64 (m, 2H), 1.58 (m, 1H), 1.52-1.43 (m, 2H), 1.11 (d, J=6.4 Hz, 3H). Mass (m/z): 352.2 [M+H]⁺.

### Compound 277

### 5-((3,5-bis(dimethylamino)phenyl)amino)-1,3-dimethyl-1,3-dihydro-2H-benzo[d]imidazol-2-one

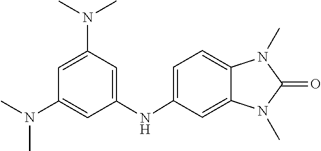 [SMILES: CN(C)c1cc(Nc2ccc3c(c2)n(C)c(=O)n3C)cc(N(C)C)c1]

The title compound 277 (13.3 mg) was prepared in a total yield of 78.5% as a gray solid from N¹,N¹,N3,N3-tetramethylbenzene-1,3,5-triamine (11.6 mg, 0.065 mmol), 5-bromo-2-methylisoindoline-1,3-dione (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.44-6.57 (m, 6H), 3.39 (s, 3H), 3.36 (s, 3H), 3.22 (s, 12H). Mass (m/z): 340.2 [M+H]⁺.

### Compound 278

### 5-((2,6-dimethyl-4-(4-methylpiperidin-1-yl)phenyl)amino)-1,3-dimethyl-1,3-dihydro-2H-benzo[d]imidazol-2-one

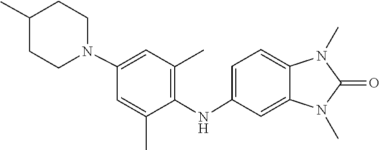 [SMILES: Cc1cc(N2CCC(C)CC2)cc(C)c1Nc1ccc2c(c1)n(C)c(=O)n2C]

The title compound 278 (12.4 mg) was prepared in a total yield of 65.6% as a gray solid from 2,6-dimethyl-4-(4-methylpiperidin-1-yl)aniline (14.2 mg, 0.065 mmol), 5-bromo-2-methylisoindoline-1,3-dione (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.33-7.05 (m, 2H), 6.83 (s, 1H), 6.26 (s, 1H), 6.05 (s, 1H), 3.66-3.54 (m, 4H), 3.23 (s, 3H), 3.19 (s, 3H), 2.14 (s, 6H), 1.97-1.86 (m, 2H), 1.70 (m, 1H), 1.49-1.39 (m, 2H), 0.98 (d, J=6.4 Hz, 3H). Mass (m/z): 379.3 [M+H]⁺.

### Compound 279

### 5-((4-(2,6-dimethylmorpholino)phenyl)amino)-1,3-dimethyl-1,3-dihydro-2H-benzo[d]imidazol-2-one

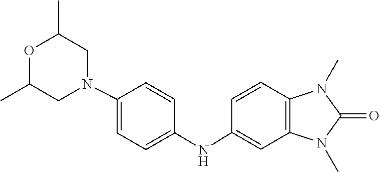 [SMILES: CC1CN(c2ccc(Nc3ccc4c(c3)n(C)c(=O)n4C)cc2)CC(C)O1]

The title compound 279 (9.8 mg) was prepared in a total yield of 12.9% as a blue solid from 4-(2,6-dimethylmorpholino)aniline (56 mg, 0.271 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, Methanol-d₄) δ 7.07-6.76 (m, 7H), 3.82 (ddt, J=12.8, 6.4, 3.2 Hz, 2H), 3.44 (s, 2H), 3.40 (s, 3H), 3.36 (s, 3H), 2.38-2.21 (m, 2H), 1.24 (d, J=6.4 Hz, 6H). Mass (m/z): 367.3 [M+H]⁺.

### Compound 280

### 1,3-dimethyl-5-((2-methyl-4-(4-methylpiperidin-1-yl)phenyl)amino)-1,3-dihydro-2H-benzo[d]imidazol-2-one

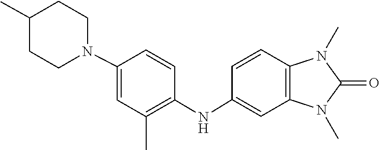 [SMILES: Cc1cc(N2CCC(C)CC2)ccc1Nc1ccc2c(c1)n(C)c(=O)n2C]

The title compound 280 (9.2 mg) was prepared in a total yield of 12.1% as a blue solid from 2-methyl-4-(4-methylpiperidin-1-yl)aniline (55 mg, 0.271 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, Methanol-d₄) δ 7.00-6.88 (m, 3H), 6.59-6.49 (m, 2H), 3.38 (s, 3H), 3.34 (s, 3H), 3.03 (d, J=11.8 Hz, 2H), 2.79 (t, J=12.4 Hz, 1H), 2.63 (t, J=11.6 Hz, 2H), 2.29 (s, 3H), 1.01 (dd, J=14.8, 6.4 Hz, 4H), 1.02 (d, J=6.0 Hz, 3H). Mass (m/z): 365.3 [M+H]⁺.

### Compound 281

### 5-((2,3-dimethyl-4-(4-methylpiperidin-1-yl)phenyl)amino)-1,3-dimethyl-1,3-dihydro-2H-benzo[d]imidazol-2-one

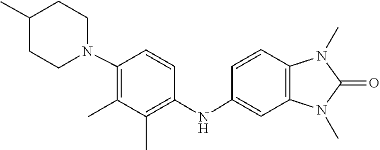 [SMILES: Cc1c(Nc2ccc3c(c2)n(C)c(=O)n3C)ccc(N2CCC(C)CC2)c1C]

The title compound 281 (5.0 mg) was prepared in a total yield of 6.4% as a yellow solid from 2,3-dimethyl-4-(4-methylpiperidin-1-yl)aniline (59 mg, 0.271 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, Methanol-d₄) δ 7.00-6.88 (m, 3H), 6.59-6.49 (m, 2H), 3.38 (s, 3H), 3.34 (s, 3H), 3.03 (d, J=11.8 Hz, 2H), 2.79 (t, J=12.4 Hz, 1H), 2.63 (t, J=11.6 Hz, 2H), 2.29 (s, 3H), 2.16 (S, 3H), 1.01 (dd, J=14.8, 6.4 Hz, 4H), 1.02 (d, J=6.0 Hz, 3H). Mass (m/z): 379.3 [M+H]⁺.

### Compound 282

### 1,3-dimethyl-5-((3-methyl-4-(4-methylpiperidin-1-yl)phenyl)amino)-1,3-dihydro-2H-benzo[d]imidazol-2-one

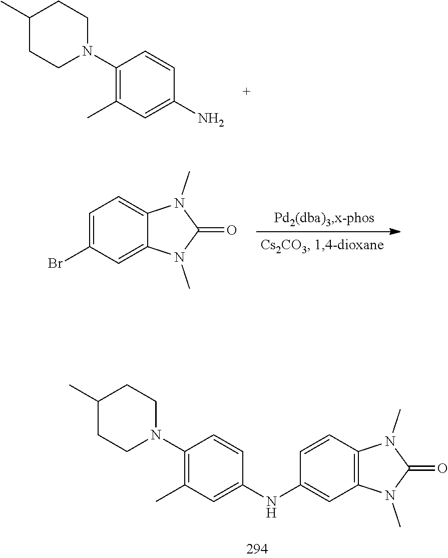 [SMILES: Cc1cc(N)ccc1N1CCC(C)CC1 ; Cn1c(=O)n(C)c2cc(Br)ccc21 ; Cc1cc(Nc2ccc3c(c2)n(C)c(=O)n3C)ccc1N1CCC(C)CC1]

The title compound 282 (8.7 mg) was prepared in a total yield of 11.5% as a yellow solid from 3-methyl-4-(4-methylpiperidin-1-yl)aniline (55 mg, 0.271 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, Methanol-d₄) δ 7.00-6.88 (m, 3H), 6.59-6.49 (m, 2H), 3.38 (s, 3H), 3.34 (s, 3H), 3.03 (d, J=11.8 Hz, 2H), 2.79 (t, J=12.4 Hz, 1H), 2.63 (t, J=11.6 Hz, 2H), 2.16 (S, 3H), 1.01 (dd, J=14.8, 6.4 Hz, 4H), 1.02 (d, J=6.0 Hz, 3H). Mass (m/z): 365.3 [M+H]⁺.

### Compound 283

### 5-((3-fluoro-4-(4-methylpiperidin-1-yl)phenyl)amino)-1,3-dimethyl-1,3-dihydro-2H-benzo[d]imidazol-2-one

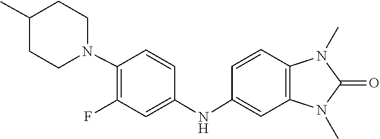 [SMILES: CC1CCN(c2ccc(Nc3ccc4c(c3)n(C)c(=O)n4C)cc2F)CC1]

The title compound 283 (3.9 mg) was prepared in a total yield of 5.1% as a yellow solid from 3-fluoro-4-(4-methylpiperidin-1-yl)aniline (56 mg, 0.271 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, Methanol-d₄) δ 7.07-7.03 (m, 1H), 6.98 (t, J=9.3 Hz, 1H), 6.90 (dd, J=6.4, 2.4 Hz, 2H), 6.79-6.72 (m, 2H), 3.42 (s, 3H), 3.39 (s, 3H), 2.66 (t, J=11.2 Hz, 2H), 2.21 (t, J=7.6 Hz, 1H), 2.05 (d, J=6.0 Hz, 1H), 1.76 (d, J=12.4 Hz, 2H), 1.48-1.39 (m, 3H), 1.02 (d, J=6.0 Hz, 3H). Mass (m/z): 369.3 [M+H]⁺.

### Compound 284

### 5-((4-(dimethylamino)phenyl)amino)-1,3-dimethyl-1,3-dihydro-2H-benzo[d]imidazol-2-one

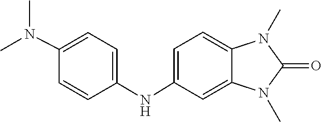 [SMILES: CN(C)c1ccc(Nc2ccc3c(c2)n(C)c(=O)n3C)cc1]

The title compound 284 (12.1 mg) was prepared in a yield of 11.12% as a gray powder from N¹,N¹-dimethylbenzene-1,4-diamine (50 mg, 0.37 mmol) and 5-bromo-1,3-dimethyl-1,3-dihydro-2H-benzo[d]imidazol-2-one (88 mg, 0.37 mmol) according to the procedure for compound 1. ¹H NMR (400 MHz, DMSO-d₆) δ 7.42 (s, 1H), 6.93 (s, 3H), 6.66 (s, 5H), 3.22 (s, 6H), 2.81 (s, 6H). Mass (m/z): 297.5 [M+H]⁺.

### Compound 285

### 1-methyl-N-(4-(4-methylpiperidin-1-yl)phenyl)-1H-benzo[d]imidazol-5-amine

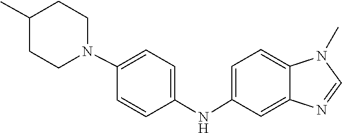 [SMILES: CC1CCN(c2ccc(Nc3ccc4c(c3)ncn4C)cc2)CC1]

The title compound 285 (14.0 mg) was prepared in a yield of 16.63% as a violet powder from 4-(4-methylpiperidin-1-yl)aniline (50 mg, 0.26 mmol) and 5-bromo-1-methyl-1H-benzo[d]imidazole (55 mg, 0.26 mmol) according to the procedure for compound 1. ¹H NMR (DMSO-d₆) δ 8.08 (s, 1H), 7.84 (s, 1H), 7.51 (s, 1H), 6.95 (d, J=58.4 Hz, 6H), 3.74 (s, 3H), 3.50 (s, 2H), 2.56 (s, 1H), 1.69 (s, 2H), 1.45 (s, 1H), 1.24 (s, 3H), 0.94 (d, J=6.4 Hz, 3H). Mass (m/z): 321.6 [M+H]⁺.

### Compound 286

### 3-methyl-5-((4-(4-methylpiperidin-1-yl)phenyl)amino)benzo[d]oxazol-2(3H)-one

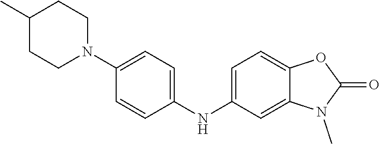 [SMILES: CC1CCN(c2ccc(Nc3ccc4oc(=O)n(C)c4c3)cc2)CC1]

The title compound 286 (5.1 mg) was prepared in a yield of 5.75% as a wheat powder from 4-(4-methylpiperidin-1-yl)aniline (50 mg, 0.26 mmol) and 5-bromo-3-methylbenzo[d]oxazol-2(3H)-one (60 mg, 0.26 mmol) according to the procedure for compound 1. ¹H NMR (DMSO-d₆) δ 7.80 (s, 1H), 7.11 (d, J=8.7 Hz, 1H), 7.04-6.80 (m, 4H), 6.73 (s, 1H), 6.61 (d, J=8.5 Hz, 1H), 3.51 (d, J=11.8 Hz, 2H), 3.26 (s, 3H), 2.59 (s, 1H), 1.69 (d, J=12.6 Hz, 2H), 1.45 (s, 1H), 1.25 (d, J=9.0 Hz, 3H), 0.94 (d, J=6.5 Hz, 3H). Mass (m/z): 338.3 [M+H]⁺.

### Compound 287

### 1,3-dimethyl-5-((4-(4-methylpiperidin-1-yl)phenyl)amino)-1,3-dihydro-2H-benzo[d]imidazol-2-one

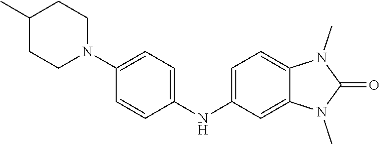 [SMILES: CC1CCN(c2ccc(Nc3ccc4c(c3)n(C)c(=O)n4C)cc2)CC1]

The title compound 287 (13.9 mg) was prepared in a total yield of 79.2% as a yellow solid from 4-(4-methylpiperidin-1-yl)aniline (12.4 mg, 0.065 mmol), 5-bromo-2-methylisoindoline-1,3-dione (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, DMSO-d₆) δ 7.46 (d, J=8.4 Hz, 2H), 7.08 (d, J=8.4 Hz, 1H), 7.01 (d, J=8.4 Hz, 2H), 6.93 (s, 1H), 6.84 (d, J=8.4 Hz, 1H), 3.60-3.42 (m, 4H), 3.32 (s, 3H), 3.29 (s, 3H), 1.96-1.84 (m, 2H), 1.76 (m, 1H), 1.65-1.48 (m, 2H), 0.98 (d, J=6.4 Hz, 3H). Mass (m/z): 351.3 [M+H]⁺.

### Compound 288

### 1,3-dimethyl-5-((4-methyl-3,4-dihydro-2H-benzo[b][1,4]oxazin-7-yl) amino)-1,3-dihydro-2H-benzo[d]imidazol-2-one

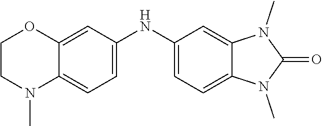 [SMILES: CN1CCOc2cc(Nc3ccc4c(c3)n(C)c(=O)n4C)ccc21]

The title compound 288 (30.5 mg) was prepared in a total yield of 37.7% as a gray solid from 5-amino-1,3-dimethyl-1,3-dihydro-2H-benzo[d]imidazol-2-one (89 mg, 0.50 mmol), 7-bromo-4-methyl-3,4-dihydro-2H-benzo[b][1,4]oxazine (57 mg, 0.25 mmol), Pd₂(dba)₃ (2.3 mg, 2.5 umol), X-Phos (6.0 mg, 12.5 umol), and t-BuONa (36 mg, 0.38 mmol) according to the procedure for compound 1. ¹H NMR (400 MHz, DMSO-d₆) δ 7.50 (s, 1H), 7.01-6.90 (m, 1H), 6.77-6.59 (m, 3H), 6.54 (s, 1H), 6.42 (s, 1H), 4.28-4.15 (m, 2H), 3.27 (s, 3H), 3.25 (s, 3H), 3.17-3.04 (m, 2H), 2.75 (s, 3H). Mass (m/z): 325.2 [M+H]⁺.

### Compound 289

### 1,3-dimethyl-5-((1-methylindolin-5-yl)amino)-1,3-dihydro-2H-benzo[d]imidazol-2-one

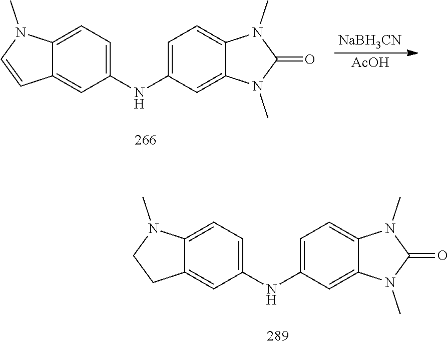

To a solution of 1,3-dimethyl-5-((1-methyl-1H-indol-5-yl)amino)-1,3-dihydro-2H-benzo[d]imidazol-2-one (70 mg, 0.23 mmol) in AcOH (2.0 mL) was added NaBH₃CN (29 mg, 0.46 mmol). Then the mixture was stirred overnight at r.t. 5 mL of water was added. The PH of the filtration was adjusted to 8-9 with sodium carbonate solution. Then the mixture was extracted by DCM (15 mL×3). The combined organic layers were washed with water (10 mL), dried over Na₂SO₄ and concentrated. The residue was purified by perp-HPLC to afford the desired product as a blue solid. ¹H NMR (400 MHz, DMSO-d₆) δ 7.49 (s, 1H), 7.06-6.98 (m, 1H), 6.77-6.59 (m, 3H), 6.54 (s, 1H), 4.28-4.15 (m, 2H), 3.34 (s, 3H), 3.27 (s, 3H), 3.25 (s, 3H), 3.17-3.04 (m, 2H). Mass (m/z): 309.2 [M+H]⁺.

### Compound 290

### 5-((4-ethynylphenyl)amino)-1,3-dimethyl-1,3-dihydro-2H-benzo[d]imidazol-2-one

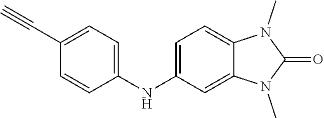 [SMILES: C#Cc1ccc(Nc2ccc3c(c2)n(C)c(=O)n3C)cc1]

The title compound 290(6.6 mg) was prepared in a total yield of 7.2% as a gray solid from 4-ethynylaniline (90 mg, 0.38 mmol), 5-bromo-1,3-dimethyl-2,3-dihydro-1H-1,3-benzodiazol-2-one (70 mg, 0.29 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 7.29-7.21 (m, 4H), 7.12 (d, J=8.0 Hz, 1H), 6.71-6.65 (m, 2H), 3.43 (d, J=1.4 Hz, 6H). Mass (m/z): 278.2 [M+H]⁺.

### Compound 291

### 1-imino-2-methyl-N-(4-(4-(trifluoromethyl)piperidin-1-yl)phenyl) isoindolin-5-amine

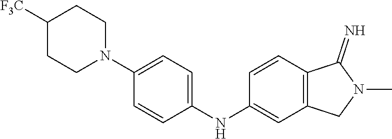 [SMILES: CN1Cc2cc(Nc3ccc(N4CCC(C(F)(F)F)CC4)cc3)ccc2C1=N]

The title compound 291 (3.3 mg) was prepared in a total yield of 3.4% as a gray solid from 4-(4-(trifluoromethyl)piperidin-1-yl)aniline (122 mg, 0.50 mmol), 5-bromo-2-methylisoindolin-1-imine (56 mg, 0.25 mmol), Pd₂(dba)₃ (2.3 mg, 2.5 umol), X-Phos (5.9 mg, 12.5 umol), and t-BuONa (36 mg, 0.38 mmol) according to the procedure for compound 1. ¹H NMR (400 MHz, Methanol-d₄) δ 7.69-7.59 (m, 1H), 7.12-6.82 (m, 6H), 4.60 (s, 2H), 3.74-3.56 (m, 2H), 3.24 (s, 3H), 2.74-2.59 (m, 2H), 2.30-2.20 (m, 1H), 1.94-1.85 (m, 2H), 1.67-1.57 (m, 2H). Mass (m/z): 389.3 [M+H]⁺.

### Compound 292

### 1,3-dimethyl-5-((4-(pyrrolidine-1-carbonyl)phenyl)amino)-1,3-dihydro-2H-benzo[d]imidazol-2-one

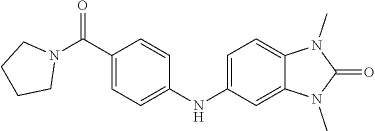 [SMILES: Cn1c(=O)n(C)c2cc(Nc3ccc(C(=O)N4CCCC4)cc3)ccc21]

The title compound 292 (14.5 mg) was prepared in a total yield of 20.7% as a light green solid from 5-amino-1,3-dimethyl-1,3-dihydro-2H-benzo[d]imidazol-2-one (71 mg, 0.40 mmol), (4-bromophenyl) (pyrrolidin-1-yl)methanone (63 mg, 0.25 mmol), Pd₂(dba)₃ (1.8 mg, 2 umol), X-Phos (4.8 mg, 10.0 umol), and t-BuONa (29 mg, 0.30 mmol) according to the procedure for compound 1. ¹H NMR (400 MHz, Methanol-d₄) δ 7.36-7.27 (m, 2H), 6.98 (d, J=8.8 Hz, 1H), 6.89 (dt, J=6.9, 2.2 Hz, 4H), 3.52-3.44 (m, 4H), 3.31 (s, 3H), 3.29 (s, 3H), 1.94-1.74 (m, 4H). Mass (m/z): 351.2 [M+H]⁺.

### Compound 293

### 1,3,3-trimethyl-5-((4-(4-(trifluoromethyl)piperidin-1-yl)phenyl) amino)indolin-2-one

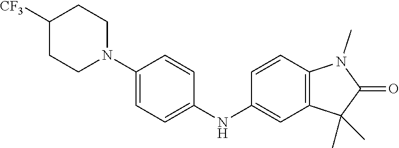 [SMILES: CN1C(=O)C(C)(C)c2cc(Nc3ccc(N4CCC(C(F)(F)F)CC4)cc3)ccc21]

The title compound 293 (11.1 mg) was prepared in a total yield of 9.0% as a light gray powder from 4-(4-(trifluoromethyl)piperidin-1-yl)aniline (122 mg, 0.50 mmol), 5-bromo-1,3,3-trimethylindolin-2-one (84 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 6.94-6.82 (m, 6H), 6.76 (d, J=8.2 Hz, 1H), 3.56-3.44 (m, 2H), 3.09 (s, 3H), 2.63-2.49 (m, 2H), 2.25-2.10 (m, 1H), 1.95-1.83 (m, 2H), 1.63 (qd, J=12.5, 4.1 Hz, 3H), 1.22 (s, 6H). Mass (m/z): 418.2 [M+H]⁺.

### Compound 294

### 1-imino-2-methyl-N-(4-(4-methylpiperidin-1-yl)phenyl)isoindolin-5-amine

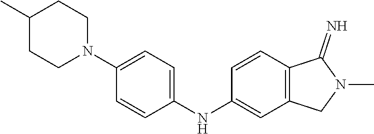

The title compound 294 (11.1 mg) was prepared in a total yield of 10.0% as a yellow powder from 4-(4-methylpiperidin-1-yl)aniline (95 mg, 0.50 mmol), 5-bromo-2-methylisoindolin-1-imine (75 mg, 0.33 mmol), Pd₂(dba)₃ (3.0 mg, 3.3 umol), X-Phos (7.8 mg, 16.5 umol), and t-BuONa (48 mg, 0.5 mmol) according to the procedure for compound 1. ¹H NMR (400 MHz, Methanol-d₄) δ 7.62 (d, J=8.6 Hz, 1H), 7.05-7.00 (m, 2H), 6.95-6.85 (m, 4H), 4.59 (s, 2H), 3.50 (dt, J=12.7, 3.3 Hz, 2H), 2.58 (td, J=12.1, 2.6 Hz, 2H), 1.73-1.64 (m, 2H), 1.48-1.38 (m, 1H), 1.26 (qd, J=12.1, 3.9 Hz, 2H), 0.90 (d, J=6.5 Hz, 3H). Mass (m/z): 335.3 [M+H]⁺

### Compound 295

### 1,3-dimethyl-5-((4-(pyrrolidin-1-ylmethyl)phenyl)amino)-1,3-dihydro-2H-benzo[d]imidazol-2-one

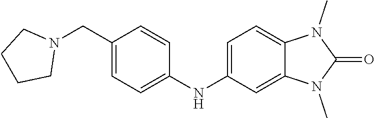 [SMILES: Cn1c(=O)n(C)c2cc(Nc3ccc(CN4CCCC4)cc3)ccc21]

The title compound 295 (31.3 mg) was prepared in a total yield of 37.2% as a Rosy Brown green solid from 1-(4-bromobenzyl)pyrrolidine (71 mg, 0.40 mmol), 5-amino-1,3-dimethyl-1,3-dihydro-2H-benzo[d]imidazol-2-one (60 mg, 0.25 mmol), Pd₂(dba)₃ (1.8 mg, 2 umol), X-Phos (4.8 mg, 10.0 umol), and t-BuONa (29 mg, 0.30 mmol) according to the procedure for compound 1. ¹H NMR (400 MHz, Methanol-d₄) δ 7.17-7.11 (m, 2H), 6.98-6.88 (m, 3H), 6.86-6.81 (m, 2H), 3.86 (s, 2H), 3.30 (s, 3H), 3.27 (s, 3H), 2.95-2.85 (m, 4H), 1.91-1.83 (m, 4H). Mass (m/z): 337.2 [M+H]⁺.

### Compound 296

### 5-((3-methoxy-4-(pentyloxy)phenyl)amino)-1,3-dimethyl-1,3-dihydro-2H-benzo[d]imidazol-2-one

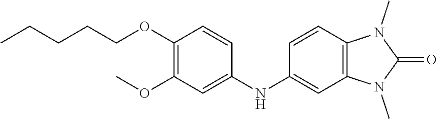 [SMILES: CCCCCOc1ccc(Nc2ccc3c(c2)n(C)c(=O)n3C)cc1OC]

The title compound 296 (7.4 mg) was prepared in a total yield of 9.6% as a yellow solid from 3-methoxy-4-(pentyloxy)aniline (56 mg, 0.271 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol) and Cs₂CO₃ (102 mg, 0.312 mmol) and 1,4-dioxane (5 mL) according to the procedure for compound 1. ¹H NMR (400 MHz, DMSO-d₆) δ 7.71 (s, 1H), 6.99 (d, J=8.3 Hz, 1H), 6.81 (dd, J=5.3, 3.3 Hz, 2H), 6.73 (dd, J=8.3, 2.1 Hz, 1H), 6.66 (d, J=2.6 Hz, 1H), 6.53 (dd, J=8.5, 2.5 Hz, 1H), 3.85 (t, J=6.6 Hz, 2H), 3.70 (s, 2H), 3.32 (s, 3H), 3.28 (s, 3H), 3.26 (s, 3H), 1.67 (p, J=6.8 Hz, 2H), 1.42-1.31 (m, 4H), 0.90 (t, J=7.0 Hz, 3H). Mass (m/z): 370.3 [M+H]⁺.

### Compound 297

### 5-((4-(4,4-difluoropiperidin-1-yl)phenyl)amino)-1,3-dimethyl-1,3-dihydro-2H-benzo[d]imidazol-2-one

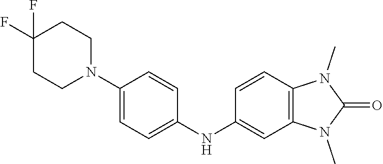 [SMILES: Cn1c(=O)n(C)c2cc(Nc3ccc(N4CCC(F)(F)CC4)cc3)ccc21]

The title compound 297 (10.4 mg) was prepared in a total yield of 13.4% as a blue solid from 4-(4,4-difluoropiperidin-1-yl)aniline (58 mg, 0.271 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, DMSO-d₆) δ 6.89-6.79 (m, 5H), 6.68 (d, J=2.0 Hz, 1H), 6.62 (dd, J=8.4, 2.0 Hz, 1H), 3.21 (s, 3H), 3.18 (s, 3H), 3.12 (d, J=10.0 Hz, 2H), 2.85-2.93 (m, 2H), 1.92 (dd, J=10.4, 5.2 Hz, 2H), 1.75 (d, J=10.4 Hz, 2H). Mass (m/z): 373.3 [M+H]⁺.

### Compound 298

### 5-((3-chloro-4-(4-methylpiperidin-1-yl)phenyl)amino)-1,3-dimethyl-1,3-dihydro-2H-benzo[d]imidazol-2-one

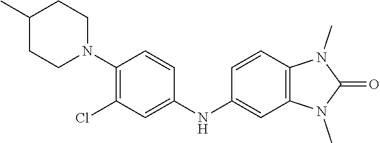 [SMILES: CC1CCN(c2ccc(Nc3ccc4c(c3)n(C)c(=O)n4C)cc2Cl)CC1]

The title compound 298 (15.2 mg) was prepared in a total yield of 19.0% as a white solid from 3-chloro-4-(4-methylpiperidin-1-yl)aniline (60 mg, 0.271 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, DMSO-d₆) δ 7.92 (s, 1H), 6.96 (t, J=8.8 Hz, 2H), 6.90 (d, J=2.4 Hz, 1H), 6.84 (dd, J=8.8, 2.6 Hz, 1H), 6.78 (d, J=2.0 Hz, 1H), 6.71 (dd, J=8.4, 2.0 Hz, 1H), 3.26 (s, 1H), 3.23 (s, 3H), 3.21 (s, 3H), 3.01 (d, J=11.6 Hz, 2H), 2.48 (d, J=2.4 Hz, 1H), 1.66-1.56 (m, 2H), 1.42-1.32 (m, 1H), 1.22 (dd, J=12.0, 3.6 Hz, 2H), 0.88 (d, J=6.4 Hz, 3H). Mass (m/z): 386.3 [M+H]⁺.

### Compound 299

### 5-((4-(4-fluoropiperidin-1-yl)phenyl)amino)-1,3-dimethyl-1,3-dihydro-2H-benzo[d]imidazol-2-one

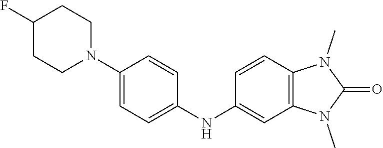 [SMILES: Cn1c(=O)n(C)c2cc(Nc3ccc(N4CCC(F)CC4)cc3)ccc21]

The title compound 299 (10.3 mg) was prepared in a total yield of 14.1% as a white solid from 4-(4-fluoropiperidin-1-yl)aniline (49 mg, 0.271 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, DMSO-d₆) δ 6.89-6.79 (m, 5H), 6.68 (d, J=2.0 Hz, 1H), 6.62 (dd, J=8.4, 2.0 Hz, 1H), 3.21 (s, 3H), 3.18 (s, 3H), 3.12 (d, J=10.0 Hz, 2H), 2.91 (td, J=8.4, 7.6, 4.0 Hz, 3H), 1.92 (dd, J=10.4, 5.2 Hz, 2H), 1.75 (d, J=10.4 Hz, 2H). Mass (m/z): 355.3 [M+H]⁺.

### Compound 300

### 1-methyl-N-(4-(4-(trifluoromethyl)piperidin-1-yl)phenyl)-1H-benzo[d][1,2,3]triazol-5-amine

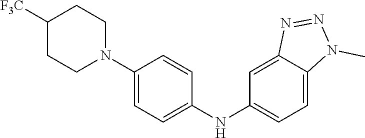 [SMILES: Cn1nnc2cc(Nc3ccc(N4CCC(C(F)(F)F)CC4)cc3)ccc21]

The title compound 300 (19.7 mg) was prepared in a total yield of 22.2% as a white solid from 4-(4-(trifluoromethyl)piperidin-1-yl)aniline (69 mg, 0.283 mmol), 5-bromo-1-methyl-1H-benzo[d][1,2,3]triazole (50 mg, 0.236 mmol), Pd₂(dba)₃ (2 mg, 0.002 mmol), X-phos (6 mg, 0.01 mmol), Cs₂CO₃ (116 mg, 0.354 mmol) and 1,4-dioxane (5 mL) according to the procedure for compound 1. ¹H NMR (400 MHz, DMSO-d₆) δ 8.00 (s, 1H), 7.59 (d, J=8.8 Hz, 1H), 7.23 (d, J=2.0 Hz, 1H), 7.15 (dd, J=8.8, 2.0 Hz, 1H), 7.03-6.97 (m, 2H), 6.90-6.84 (m, 2H), 4.15 (s, 3H), 3.59 (d, J=12.0 Hz, 2H), 2.58 (td, J=12.4, 2.6 Hz, 2H), 1.82 (d, J=12.4 Hz, 2H), 1.52 (qd, J=12.4, 4.0 Hz, 2H). Mass (m/z): 376.3 [M+H]⁺.

### Compound 301

### 4-methoxy-2-methyl-5-((4-(4-(trifluoromethyl)piperidin-1-yl)phenyl) amino)isoindolin-1-one

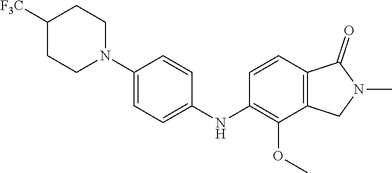 [SMILES: COc1c(Nc2ccc(N3CCC(C(F)(F)F)CC3)cc2)ccc2c1CN(C)C2=O]

The title compound 301 (44.6 mg) was prepared in a total yield of 54.5% as a white solid from 4-(4-(trifluoromethyl)piperidin-1-yl)aniline (57 mg, 0.234 mmol), 5-bromo-4-methoxy-2-methylisoindolin-1-one (50 mg, 0.195 mmol), Pd₂(dba)₃ (2 mg, 0.002 mmol), X-phos (6 mg, 0.01 mmol), Cs₂CO₃ (96 mg, 0.293 mmol) and 1,4-dioxane (5 mL) according to the procedure for compound 1. ¹H NMR (400 MHz, DMSO-d₆) δ 7.50 (s, 1H), 7.10 (d, J=8.0 Hz, 1H), 7.04-7.00 (m, 2H), 6.93-6.85 (m, 3H), 4.49 (s, 2H), 3.81 (s, 3H), 3.62 (d, J=12.4 Hz, 2H), 2.95 (s, 3H), 2.60 (td, J=12.4, 2.4 Hz, 2H), 1.86-1.76 (m, 2H), 1.50 (qd, J=12.4, 4.0 Hz, 2H). Mass (m/z): 420.3 [M+H]⁺.

### Compound 302

### 2-imino-1,3-dimethyl-N-(4-(4-(trifluoromethyl)piperidin-1-yl)phenyl)-2,3-dihydro-1H-benzo[d]imidazol-5-amine

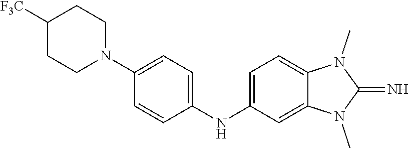 [SMILES: Cn1c(=N)n(C)c2cc(Nc3ccc(N4CCC(C(F)(F)F)CC4)cc3)ccc21]

The title compound 302 (16.2 mg) was prepared in a total yield of 80.2% as a yellow solid from 4-(4-(trifluoromethyl)piperidin-1-yl)aniline (15.9 mg, 0.065 mmol), 5-bromo-1,3-dimethyl-1,3-dihydro-2H-benzo[d]imidazol-2-imine (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.35-6.71 (m, 7H), 3.76-3.66 (m, 2H), 3.59 (s, 3H), 3.56 (s, 3H), 2.79-2.61 (m, 2H), 2.11 (m, 1H), 1.99-1.86 (m, 2H), 1.75-1.69 (m, 2H). Mass (m/z): 404.2 [M+H]⁺.

### Compound 303

### 5-((3-(dimethylamino)-4-(piperidin-1-yl)phenyl)amino)-2-methylisoindolin-1-one

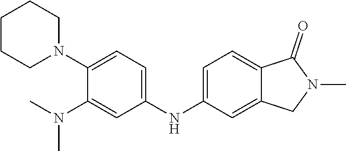 [SMILES: CN1Cc2cc(Nc3ccc(N4CCCCC4)c(N(C)C)c3)ccc2C1=O]

The title compound 303 (14.5 mg) was prepared in a total yield of 79.7% as a white solid from N¹,N¹-dimethyl-6-(piperidin-1-yl) benzene-1,3-diamine (14.2 mg, 0.065 mmol), 5-bromo-2-methylisoindolin-1-one (11.3 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.67-7.57 (m, 2H), 7.41 (d, J=2.4 Hz, 1H), 7.31-7.23 (m, 2H), 7.18 (dd, J=8.4, 2.4 Hz, 1H), 4.44 (s, 2H), 3.25-3.18 (m, 4H), 3.16 (s, 3H), 3.03 (s, 6H), 1.74-1.63 (m, 4H), 1.59-1.51 (m, 2H). Mass (m/z): 365.2 [M+H]⁺.

### Compound 304

### 6-((3-(dimethylamino)-4-(piperidin-1-yl)phenyl)amino)-2-methyl-1,2-dihydro-3H-indazol-3-one

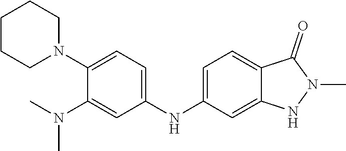 [SMILES: CN(C)c1cc(Nc2ccc3c(=O)n(C)[nH]c3c2)ccc1N1CCCCC1]

The title compound 304 (15.3 mg) was prepared in a total yield of 83.8% as a light pink solid from tert-butyl 6-((3-(dimethylamino)-4-(piperidin-1-yl)phenyl)amino)-2-methyl-3-oxo-2,3-dihydro-1H-indazole-1-carboxylate (23.3 mg, 0.05 mmol) and 3 ml HCl (4.0 M) in 1,4-dioxane according to the procedure for compound 203. ¹H NMR (400 MHz, Methanol-d₄) δ 7.80-7.26 (m, 5H), 6.97 (s, 1H), 3.25 (s, 3H), 3.20-3.09 (m, 4H), 3.02 (s, 6H), 1.86-1.68 (m, 4H), 1.56-1.34 (m, 2H). Mass (m/z): 366.2 [M+H]⁺.

### Compound 305

### 5-((4-(1H-imidazol-1-yl)phenyl)amino)-1,3-dimethyl-1,3-dihydro-2H-benzo[d]imidazol-2-one

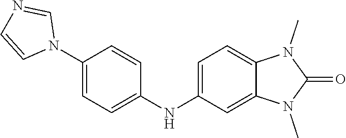 [SMILES: Cn1c(=O)n(C)c2cc(Nc3ccc(-n4ccnc4)cc3)ccc21]

The title compound 305 (42.0 mg) was prepared in a total yield of 39.3% as a light blue powder from 4-(1H-Imidazol-1-yl)aniline (67 mg, 0.43 mmol), 5-bromo-1,3-dimethyl-2,3-dihydro-1H-1,3-benzodiazol-2-one (80 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 9.20 (s, 1H), 7.84 (t, J=1.8 Hz, 1H), 7.62 (t, J=1.7 Hz, 1H), 7.42-7.32 (m, 2H), 7.06-6.98 (m, 3H), 6.94-6.86 (m, 2H), 3.32 (s, 3H), 3.29 (s, 3H). Mass (m/z): 320.3 [M+H]⁺.

### Compound 306

### ethyl 4-((1,3-dimethyl-2-oxo-2,3-dihydro-1H-benzo[d]imidazol-5-yl) amino)benzoate

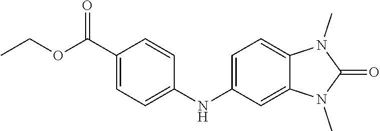 [SMILES: CCOC(=O)c1ccc(Nc2ccc3c(c2)n(C)c(=O)n3C)cc1]

The title compound 306 (32.7 mg) was prepared in a total yield of 30.0% as a light yellow powder from 4-(ethoxycarbonyl)phenylamine (71 mg, 0.43 mmol), 5-bromo-1,3-dimethyl-2,3-dihydro-1H-1,3-benzodiazol-2-one (80 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 7.85-7.79 (m, 2H), 7.16-7.07 (m, 1H), 7.03-6.90 (m, 4H), 3.43 (s, 3H), 3.40 (s, 3H), 1.37 (t, J=7.1 Hz, 3H). Mass (m/z): 326.2 [M+H]⁺.

### Compound 307

### 5-((2,3-dihydrobenzo[b][1,4]dioxin-6-yl)amino)-1,3-dimethyl-1,3-dihydro-2H-benzo[d]imidazol-2-one

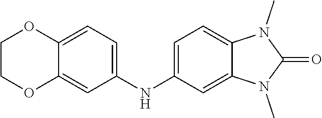 [SMILES: Cn1c(=O)n(C)c2cc(Nc3ccc4c(c3)OCCO4)ccc21]

The title compound 307 (34.9 mg) was prepared in a total yield of 33.7% as a light yellow powder from 1,4-benzodioxan-6-amine (85 mg, 0.43 mmol), 5-bromo-1,3-dimethyl-2,3-dihydro-1H-1,3-benzodiazol-2-one (80 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 7.00 (d, J=8.6 Hz, 1H), 6.84-6.77 (m, 2H), 6.72 (d, J=8.5 Hz, 1H), 6.60-6.51 (m, 2H), 4.24-4.16 (m, 4H), 3.40 (s, 3H), 3.37 (s, 3H). Mass (m/z): 312.2 [M+H]⁺.

### Compound 308

### 5-((3-(dimethylamino)phenyl)amino)-1,3-dimethyl-1,3-dihydro-2H-benzo[d]imidazol-2-one

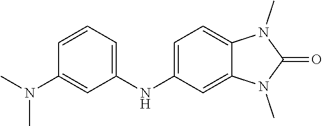 [SMILES: CN(C)c1cccc(Nc2ccc3c(c2)n(C)c(=O)n3C)c1]

The title compound 308 (12.6 mg) was prepared in a total yield of 12.8% as a light yellow powder from N,N-dimethyl-m-phenylenediamine (59 mg, 0.43 mmol), 5-bromo-1,3-dimethyl-2,3-dihydro-1H-1,3-benzodiazol-2-one (80 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 6.98-6.87 (m, 2H), 6.84-6.77 (m, 2H), 6.40-6.30 (m, 2H), 6.24-6.17 (m, 1H), 3.29 (s, 3H), 3.25 (s, 3H), 2.77 (s, 6H). Mass (m/z): 297.3 [M+H]⁺.

### Compound 309

### 5-((4-ethoxyphenyl)amino)-1,3-dimethyl-1,3-dihydro-2H-benzo[d]imidazol-2-one

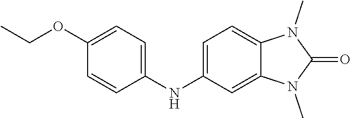 [SMILES: CCOc1ccc(Nc2ccc3c(c2)n(C)c(=O)n3C)cc1]

The title compound 309 (36.5 mg) was prepared in a total yield of 36.9% as a light blue powder from 4-ethoxybenzenamine (59 mg, 0.43 mmol), 5-bromo-1,3-dimethyl-2,3-dihydro-1H-1,3-benzodiazol-2-one (80 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 6.96-6.82 (m, 3H), 6.76-6.58 (m, 4H), 3.89 (q, J=7.0 Hz, 2H), 3.27 (s, 3H), 3.23 (s, 3H), 1.27 (t, J=7.0 Hz, 3H). Mass (m/z): 298.2 [M+H]⁺.

### Compound 310

### 5-(imidazo[1,2-a]pyridin-6-ylamino)-1,3-dimethyl-1,3-dihydro-2H-benzo[d]imidazol-2-one

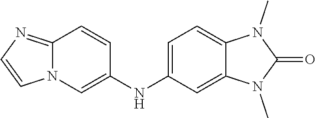 [SMILES: Cn1c(=O)n(C)c2cc(Nc3ccc4nccn4c3)ccc21]

The title compound 310 (33.9 mg) was prepared in a total yield of 34.7% as a yellow powder from H-imidazo[1,2-a]pyridin-6-amine (57 mg, 0.43 mmol), 5-bromo-1,3-dimethyl-2,3-dihydro-1H-1,3-benzodiazol-2-one (80 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 8.16 (dd, J=2.2, 0.9 Hz, 1H), 7.84 (d, J=1.9 Hz, 1H), 7.67 (d, J=1.9 Hz, 1H), 7.59 (d, J=9.6 Hz, 1H), 7.49 (dd, J=9.7, 2.2 Hz, 1H), 7.04 (d, J=8.3 Hz, 1H), 6.94 (d, J=2.0 Hz, 1H), 6.91 (dd, J=8.3, 2.1 Hz, 1H), 3.33 (s, 3H), 3.32 (s, 3H). Mass (m/z): 294.2 [M+H]⁺.

### Compound 311

### 1,3,3-trimethyl-N-(4-(4-methylpiperidin-1-yl)phenyl)indolin-5-amine

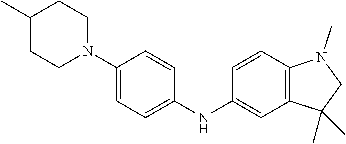

The title compound 311 (28.1 mg) was prepared in a total yield of 24.1% as a light green powder from 4-(4-methylpiperidin-1-yl)aniline (82 mg, 0.43 mmol), 5-bromo-1,3,3-trimethylindoline (80 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 8.15-5.87 (m, 7H), 3.73-3.28 (m, 2H), 3.34 (s, 3H), 2.82-2.43 (m, 2H), 1.70-1.63 (m, 2H), 1.31-1.07 (m, 8H), 0.90 (d, J=6.1 Hz, 6H), 0.83-0.74 (m, 2H). Mass (m/z): 350.4 [M+H]⁺.

### Compound 312

### 1,3-dimethyl-5-((3,4,5-trimethoxyphenyl)amino)-1,3-dihydro-2H-benzo[d]imidazol-2-one

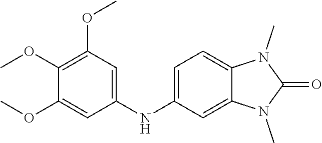 [SMILES: COc1cc(Nc2ccc3c(c2)n(C)c(=O)n3C)cc(OC)c1OC]

The title compound 312 (34.4 mg) was prepared in a total yield of 30.2% as a white powder from 3,4,5-trimethoxyaniline (79 mg, 0.43 mmol), 5-bromo-1,3-dimethyl-2,3-dihydro-1H-1,3-benzodiazol-2-one (80 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 6.97-6.90 (m, 1H), 6.83-6.78 (m, 2H), 6.22 (s, 2H), 3.66 (s, 6H), 3.60 (s, 3H), 3.29 (s, 3H), 3.26 (s, 3H). Mass (m/z): 344.2 [M+H]⁺.

### Compound 313

### 2-methyl-1-(methylimino)-N-(4-(4-(trifluoromethyl)piperidin-1-yl) phenyl)isoindolin-5-amine

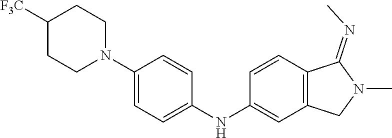 [SMILES: C/N=C1\c2ccc(Nc3ccc(N4CCC(C(F)(F)F)CC4)cc3)cc2CN1C]

The title compound 313 (9.4 mg) was prepared in a total yield of 11.2% as a white solid from 4-(4-(trifluoromethyl)piperidin-1-yl)aniline (61 mg, 0.251 mmol), 5-bromo-N,2-dimethylisoindolin-1-imine (50 mg, 0.209 mmol), Pd₂(dba)₃ (2 mg, 0.002 mmol), X-phos (6 mg, 0.01 mmol), Cs₂CO₃ (103 mg, 0.314 mmol) and 1,4-dioxane (5 mL) according to the procedure for compound 1. ¹H NMR (400 MHz, DMSO-d₆) δ 9.55 (s, 1H), 8.59 (s, 1H), 7.85 (d, J=8.4 Hz, 1H), 7.14-7.05 (m, 4H), 6.99 (d, J=8.8 Hz, 2H), 3.73 (d, J=12.4 Hz, 2H), 3.08 (s, 3H), 2.73-2.66 (m, 2H), 1.90 (d, J=12.8 Hz, 2H), 1.58 (qd, J=12.4, 4.0 Hz, 3H), 1.24 (s, 2H). Mass (m/z): 403.3 [M+H]⁺.

### Compound 314

### 1-methyl-N-(4-(4-(trifluoromethyl)piperidin-1-yl)phenyl)-1H-benzo[d][1,2,3]triazol-6-amine

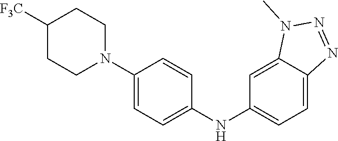 [SMILES: Cn1nnc2ccc(Nc3ccc(N4CCC(C(F)(F)F)CC4)cc3)cc21]

The title compound 314 (8.5 mg) was prepared in a total yield of 9.6% as a white solid from 4-(4-(trifluoromethyl)piperidin-1-yl)aniline (69 mg, 0.283 mmol), 6-bromo-1-methyl-1H-benzo[d][1,2,3]triazole (50 mg, 0.236 mmol), Pd₂(dba)₃ (2 mg, 0.002 mmol), X-phos (6 mg, 0.01 mmol), Cs₂CO₃ (116 mg, 0.354 mmol) and 1,4-dioxane (5 mL) according to the procedure for compound 1. ¹H NMR (400 MHz, DMSO-d₆) δ 8.31 (s, 1H), 7.77 (d, J=9.6 Hz, 1H), 7.18-7.13 (m, 2H), 7.03-6.90 (m, 4H), 4.10 (s, 3H), 3.70 (d, J=12.4 Hz, 2H), 2.68 (d, J=23.6 Hz, 2H), 1.90 (d, J=12.4 Hz, 2H), 1.59 (qd, J=12.4, 4.0 Hz, 2H). Mass (m/z): 376.3 [M+H]⁺.

### Compound 315

### 6-((4-((1R,4S)-2-azabicyclo[2.2.1]heptan-2-yl)phenyl)amino)-3-methylbenzo[d] oxazol-2(3H)-one

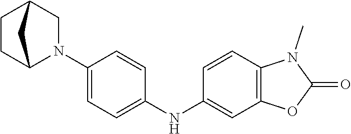 [SMILES: Cn1c(=O)oc2cc(Nc3ccc(N4C[C@H]5CC[C@@H]4C5)cc3)ccc21]

The title compound 315 (26.0 mg) was prepared in a total yield of 35.4% as a white solid from 4-((1R,4S)-2-azabicyclo[2.2.1]heptan-2-yl)aniline (50 mg, 0.263 mmol), 6-bromo-3-methylbenzo[d]oxazol-2(3H)-one (50 mg, 0.219 mmol), Pd₂(dba)₃ (2 mg, 0.002 mmol), X-phos (6 mg, 0.01 mmol), Cs₂CO₃ (108 mg, 0.329 mmol) and 1,4-dioxane (5 mL) according to the procedure for compound 1. ¹H NMR (400 MHz, DMSO-d₆) δ 7.63 (s, 1H), 7.00 (d, J=8.4 Hz, 1H), 6.93 (d, J=8.0 Hz, 2H), 6.76 (s, 1H), 6.67 (d, J=8.4 Hz, 1H), 6.48 (d, J=8.4 Hz, 2H), 4.09 (s, 1H), 2.62 (d, J=8.0 Hz, 1H), 2.54 (s, 1H), 1.69 (d, J=9.2 Hz, 1H), 1.61 (s, 2H), 1.46 (d, J=9.2 Hz, 1H), 1.25 (d, J=12.0 Hz, 2H). Mass (m/z): 336.3 [M+H]⁺.

### Compound 316

### 6-((4-(2-azaspiro[3.4]octan-2-yl)phenyl)amino)-3-methy/benzo[d]oxazol-2′3H-one

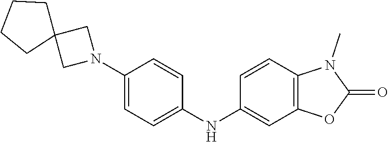 [SMILES: Cn1c(=O)oc2cc(Nc3ccc(N4CC5(CCCC5)C4)cc3)ccc21]

The title compound 316 (8.3 mg) was prepared in a total yield of 10.8% as a white solid from 4-(2-azaspiro[3.4]octan-2-yl)aniline (53 mg, 0.263 mmol), 6-bromo-3-methylbenzo[d]oxazol-2(3H)-one (50 mg, 0.219 mmol), Pd₂(dba)₃ (2 mg, 0.002 mmol), X-phos (6 mg, 0.01 mmol), Cs₂CO₃ (108 mg, 0.329 mmol) and 1,4-dioxane (5 mL) according to the procedure for compound 1. ¹H NMR (400 MHz, DMSO-d₆) δ 7.63 (s, 1H), 6.95 (d, J=8.4 Hz, 1H), 6.89-6.84 (m, 2H), 6.72 (d, J=2.0 Hz, 1H), 6.63 (dd, J=8.4, 2.1 Hz, 1H), 6.32 (d, J=8.8 Hz, 2H), 3.54 (s, 3H), 3.21 (s, 2H), 1.71 (q, J=6.0, 4.4 Hz, 4H), 1.55-1.48 (m, 4H). Mass (m/z): 350.3 [M+H]⁺.

### Compound 317

### 6-((4-(2,6-dimethylmorpholino)phenyl)amino)-3-methylbenzo[d]oxazol-2(3H)-one

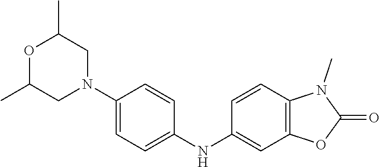 [SMILES: CC1CN(c2ccc(Nc3ccc4c(c3)oc(=O)n4C)cc2)CC(C)O1]

The title compound 317 (19.4 mg) was prepared in a total yield of 25.1% as a white solid from 4-(2,6-dimethylmorpholino)aniline (54 mg, 0.263 mmol), 6-bromo-3-methylbenzo[d]oxazol-2(3H)-one (50 mg, 0.219 mmol), Pd₂(dba)₃ (2 mg, 0.002 mmol), X-phos (6 mg, 0.01 mmol), Cs₂CO₃ (108 mg, 0.329 mmol) and 1,4-dioxane (5 mL) according to the procedure for compound 1. ¹H NMR (400 MHz, DMSO-d₆) δ 7.85 (s, 1H), 7.06 (d, J=8.4 Hz, 1H), 6.99-6.94 (m, 2H), 6.91-6.86 (m, 3H), 6.79 (dd, J=8.4, 2.0 Hz, 1H), 3.69 (ddt, J=12.4, 6.4, 3.2 Hz, 2H), 3.44 (d, J=11.2 Hz, 2H), 2.23-2.15 (m, 2H), 1.14 (d, J=6.4 Hz, 6H). Mass (m/z): 354.3 [M+H]⁺.

### Compound 318

### 5-((4-(2-methoxyethoxy)phenyl)amino)-1,3-dimethyl-1,3-dihydro-2H-benzo[d] imidazol-2-one

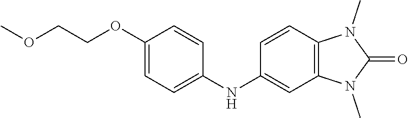 [SMILES: COCCOc1ccc(Nc2ccc3c(c2)n(C)c(=O)n3C)cc1]

The title compound 318 (18.4 mg) was prepared in a total yield of 27.0% as a white solid from 4-(2-methoxyethoxy)aniline (42 mg, 0.250 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, DMSO-d₆) δ 7.69 (s, 1H), 6.97 (dd, J=8.8, 2.4 Hz, 3H), 6.86-6.81 (m, 2H), 6.75 (d, J=2.0 Hz, 1H), 6.70 (dd, J=8.4, 2.1 Hz, 1H), 4.04-3.99 (m, 2H), 3.66-3.60 (m, 2H), 3.31 (s, 3H), 3.28 (s, 3H), 3.25 (s, 3H). Mass (m/z): 328.3 [M+H]⁺.

### Compound 319

### 5-((2-methoxypyrimidin-5-yl)amino)-1,3-dimethyl-1,3-dihydro-2H-benzo[d] imidazole-2-one

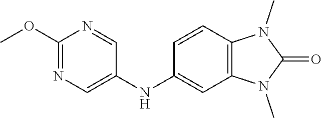 [SMILES: COc1ncc(Nc2ccc3c(c2)n(C)c(=O)n3C)cn1]

The title compound 319 (9.9 mg) was prepared in a total yield of 16.7% as a white solid from 2-methoxypyrimidin-5-amine (31 mg, 0.250 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, DMSO-d₆) δ 8.38 (s, 2H), 7.95 (s, 1H), 7.02 (d, J=8.4 Hz, 1H), 6.83 (d, J=2.0 Hz, 1H), 6.72 (dd, J=8.4, 2.0 Hz, 1H), 3.86 (s, 3H), 3.29 (d, J=4.4 Hz, 6H). Mass (m/z): 286.3 [M+H]⁺.

### Compound 320

### 5-((6-methoxypyridin-3-yl)amino)-1,3-dimethyl-1,3-dihydro-2H-benzo[d] imidazol-2-one

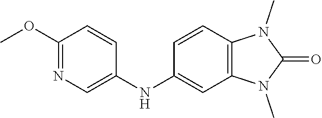 [SMILES: COc1ccc(Nc2ccc3c(c2)n(C)c(=O)n3C)cn1]

The title compound 320 (34.5 mg) was prepared in a total yield of 58.4% as a white solid from 6-methoxypyridin-3-amine (31 mg, 0.250 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), Xphos (6 mg, 0.01 mmol), Cs₂CO₃ (102 mg, 0.312 mmol) and 1,4-dioxane (5 mL) according to the procedure for compound 1. ¹H NMR (400 MHz, DMSO-d₆) δ 7.93 (d, J=2.8 Hz, 1H), 7.82 (s, 1H), 7.45 (dd, J=8.8, 2.8 Hz, 1H), 6.99 (d, J=8.4 Hz, 1H), 6.78-6.72 (m, 2H), 6.69 (dd, J=8.4, 2.0 Hz, 1H), 3.79 (s, 3H), 3.27 (d, J=7.6 Hz, 6H). Mass (m/z): 285.3 [M+H]⁺.

### Compound 321

### 1-methyl-N-(4-(4-methylpiperidin-1-yl)phenyl)-1H-benzo[d][1,2,3]triazol-6-amine

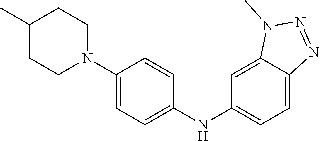 [SMILES: CC1CCN(c2ccc(Nc3ccc4nnn(C)c4c3)cc2)CC1]

The title compound 321 (55.1 mg) was prepared in a total yield of 72.6% as a white solid from 4-(4-methylpiperidin-1-yl)aniline (54 mg, 0.283 mmol), 6-bromo-1-methyl-1H-benzo[d][1,2,3]triazole (50 mg, 0.236 mmol), Pd₂(dba)₃ (2 mg, 0.002 mmol), X-phos (6 mg, 0.01 mmol), Cs₂CO₃ (116 mg, 0.354 mmol) and 1,4-dioxane (5 mL) according to the procedure for compound 1. ¹H NMR (400 MHz, DMSO-d₆) δ 8.36-8.29 (m, 1H), 7.75 (d, J=8.8 Hz, 1H), 7.15-7.10 (m, 2H), 7.01-6.97 (m, 1H), 6.97-6.91 (m, 3H), 4.09 (s, 3H), 3.57 (dt, J=12.4, 3.6 Hz, 2H), 2.60 (td, J=12.0, 2.6 Hz, 2H), 1.74-1.65 (m, 2H), 1.53-1.41 (m, 1H), 1.31-1.18 (m, 2H), 0.95 (d, J=6.4 Hz, 3H). Mass (m/z): 322.3 [M+H]⁺.

### Compound 322

### 1-methyl-N-(4-(4-methylpiperidin-1-yl)phenyl)-1H-benzo[d][1,2,3]triazol-6-amine

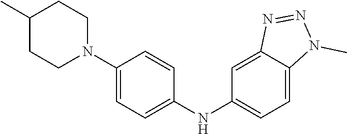

The title compound 322 (24.8 mg) was prepared in a total yield of 32.7% as a white solid from 4-(4-methylpiperidin-1-yl)aniline (54 mg, 0.283 mmol), 5-bromo-1-methyl-1H-benzo[d][1,2,3]triazole (50 mg, 0.236 mmol), Pd₂(dba)₃ (2 mg, 0.002 mmol), X-phos (6 mg, 0.01 mmol), Cs₂CO₃ (116 mg, 0.354 mmol) and 1,4-dioxane (5 mL) according to the procedure for compound 1. ¹H NMR (400 MHz, DMSO-d₆) δ 8.04 (s, 1H), 7.65 (d, J=8.8 Hz, 1H), 7.36-7.31 (m, 1H), 7.28 (d, J=2.0 Hz, 1H), 7.09-7.02 (m, 2H), 6.94-6.89 (m, 2H), 4.22 (s, 3H), 3.53 (dt, J=12.4, 3.2 Hz, 2H), 2.57 (td, J=12.0, 2.4 Hz, 2H), 1.69 (d, J=13.6 Hz, 2H), 1.45 (ddt, J=10.4, 7.2, 3.6 Hz, 1H), 1.25 (dd, J=12.4, 3.6 Hz, 2H), 0.94 (d, J=6.4 Hz, 3H). Mass (m/z): 322.3 [M+H]⁺.

### Compound 323

### 2-methyl-N-(4-(4-(trifluoromethyl)piperidin-1-yl)phenyl)benzo[d]oxazol-6-amine

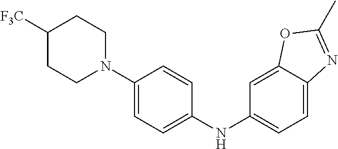 [SMILES: Cc1nc2ccc(Nc3ccc(N4CCC(C(F)(F)F)CC4)cc3)cc2o1]

The title compound 323 (61.6 mg) was prepared in a total yield of 64.1% as a white solid from 4-(4-(trifluoromethyl)piperidin-1-yl)aniline (69 mg, 0.283 mmol), 6-bromo-2-methylbenzo[d]oxazole (50 mg, 0.256 mmol), Pd₂(dba)₃ (2 mg, 0.002 mmol), X-phos (6 mg, 0.01 mmol) and Cs₂CO₃ (126 mg, 0.384 mmol) and 1,4-dioxane (5 mL) according to the procedure for compound 1. ¹H NMR (400 MHz, DMSO-d₆) δ 8.03 (dt, J=6.8, 2.8 Hz, 1H), 7.34 (d, J=8.4 Hz, 1H), 7.03 (d, J=2.0 Hz, 1H), 7.00-6.94 (m, 2H), 6.88-6.80 (m, 3H), 3.57 (d, J=12.4 Hz, 2H), 2.56 (td, J=12.4, 2.4 Hz, 2H), 2.45 (s, 3H), 2.36 (ddq, J=12.4, 8.8, 3.6 Hz, 1H), 1.86-1.76 (m, 2H), 1.51 (qd, J=12.4, 4.0 Hz, 2H). Mass (m/z): 376.3 [M+H]⁺.

### Compound 324

### 6-((4-(3,3-dimethylazetidin-1-yl)phenyl)amino)-3-methylbenzo[d]oxazol-2(3H)-one

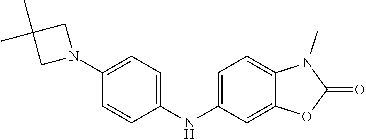 [SMILES: Cn1c(=O)oc2cc(Nc3ccc(N4CC(C)(C)C4)cc3)ccc21]

The title compound 324 (13.1 mg) was prepared in a total yield of 80.9% as a yellow solid from 4-(3,3-dimethylazetidin-1-yl)aniline (11.4 mg, 0.065 mmol), 6-bromo-3-methylbenzo[d]oxazol-2(3H)-one (11.4 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.14-6.58 (m, 7H), 3.64 (s, 4H), 3.37 (s, 3H), 1.25 (s, 6H). Mass (m/z): 324.1 [M+H]⁺.

### Compound 325

### 6-((4-(2,5-dimethylpyrrolidin-1-yl)phenyl)amino)-3-methylbenzo[d]oxazol-2(3H)-one

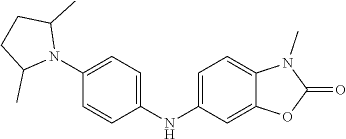 [SMILES: CC1CCC(C)N1c1ccc(Nc2ccc3c(c2)oc(=O)n3C)cc1]

The title compound 325 (11.3 mg) was prepared in a total yield of 66.7% as a white solid from 4-(2,5-dimethylpyrrolidin-1-yl)aniline (12.4 mg, 0.065 mmol), 6-bromo-3-methylbenzo[d]oxazol-2(3H)-one (11.4 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.05-6.87 (m, 3H), 6.78-6.63 (m, 2H), 6.54-6.43 (m, 2H), 3.53-3.46 (m, 2H), 3.34 (s, 3H), 2.11-1.65 (m, 4H), 1.14 (d, J=6.4 Hz, 6H). Mass (m/z): 338.2 [M+H]⁺.

### Compound 326

### 2,7-dimethyl-5-((4-(4-(trifluoromethyl)piperidin-1-yl)phenyl)amino) isoindolin-1-one

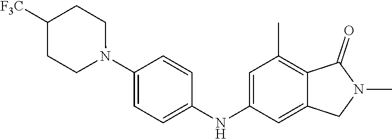 [SMILES: Cc1cc(Nc2ccc(N3CCC(C(F)(F)F)CC3)cc2)cc2c1C(=O)N(C)C2]

The title compound 326 (16.7 mg) was prepared in a total yield of 82.6% as a light yellow solid from 4-(4-(trifluoromethyl)piperidin-1-yl)aniline (15.9 mg, 0.065 mmol), 5-bromo-2,7-dimethylisoindolin-1-one (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.34-6.75 (m, 6H), 4.29 (s, 2H), 3.76-3.61 (m, 2H), 3.11 (s, 3H), 2.79-2.66 (m, 2H), 2.53 (s, 3H), 2.15 (m, 1H), 2.06-1.93 (m, 2H), 1.79-1.66 (m, 2H). Mass (m/z): 404.2 [M+H]⁺.

### Compound 327

### 6-((4-(2-azaspiro[3.3]heptan-2-yl)phenyl)amino)-3-methylbenzo[d]oxazol-2(3H)-one

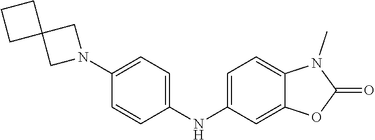 [SMILES: Cn1c(=O)oc2cc(Nc3ccc(N4CC5(CCC5)C4)cc3)ccc21]

The title compound 327 (15.8 mg) was prepared in a total yield of 78.2% as a wheat solid from 4-(2-azaspiro[3.3]heptan-2-yl)aniline (12.2 mg, 0.065 mmol), 6-bromo-3-methylbenzo[d]oxazol-2(3H)-one (11.4 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.41-6.74 (m, 6H), 3.71 (s, 4H), 3.15 (s, 3H), 2.19-2.11 (m, 4H), 1.89-1.77 (m, 2H). Mass (m/z): 404.2 [M+H]⁺.

### Compound 328

### 1,3-diethyl-5-((4-(4-methylpiperidin-1-yl)phenyl)amino)-1,3-dihydro-2H-benzo[d]imidazol-2-one

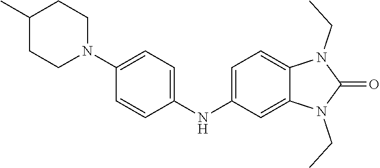 [SMILES: CCn1c(=O)n(CC)c2cc(Nc3ccc(N4CCC(C)CC4)cc3)ccc21]

The title compound 328 (1.2 mg) was prepared in a yield of 1.21% as a grey solid from 4-(4-methylpiperidin-1-yl)aniline (50 mg, 0.26 mmol) and 5-bromo-1,3-diethyl-1,3-dihydro-2H-benzo[d]imidazol-2-one (70 mg, 0.26 mmol) according to the procedure for compound 1. ¹H NMR (DMSO-d₆) δ 7.61 (s, 1H), 7.21 (s, 1H), 7.01-6.72 (m, 4H), 6.65 (s, 1H), 3.79 (s, 2H), 1.99 (q, J=6.9, 6.2 Hz, 5H), 1.65 (m, 1H), 1.45 (s, 3H), 1.19-1.13 (m, 5H), 0.94 (d, J=6.4 Hz, 2H), 0.84 (d, J=7.2 Hz, 4H). Mass (m/z): 379.3 [M+H]⁺.

### Compound 329

### 1-methyl-N-(4-(4-methylpiperidin-1-yl)phenyl)-1H-indol-5-amine

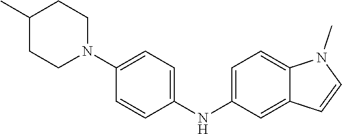 [SMILES: CC1CCN(c2ccc(Nc3ccc4c(ccn4C)c3)cc2)CC1]

The title compound 329 (1.5 mg) was prepared in a yield of 1.79% as a grey solid from 4-(4-methylpiperidin-1-yl)aniline (50 mg, 0.26 mmol) and 5-bromo-1-methyl-1H-indole (55 mg, 0.26 mmol) according to the procedure for compound 1. ¹H NMR (DMSO-d₆) δ 7.20 (s, 5H), 7.02-6.82(m, 3), 6.64 (s, 2H), 2.10-1.80 (m, 5H), 1.44 (s, 3H), 1.14 (s, 3H), 0.83 (d, J=6.8 Hz, 4H). Mass (m/z): 320.4 [M+H]⁺.

### Compound 330

### 1-methyl-N-(4-(4-methylpiperidin-1-yl)phenyl)-1H-indol-6-amine

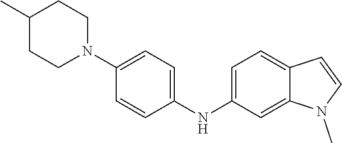 [SMILES: CC1CCN(c2ccc(Nc3ccc4ccn(C)c4c3)cc2)CC1]

The title compound 330 (2.4 mg) was prepared in a yield of 2.86% as a grey powder from 4-(4-methylpiperidin-1-yl)aniline (50 mg, 0.26 mmol) and 6-bromo-1-methyl-1H-indole (55 mg, 0.26 mmol) according to the procedure for compound 1. ¹H NMR (DMSO-d₆) δ 7.62-7.34 (m, 4H), 7.21-6.86 (m, 3H), 6.65 (s, 2H), 2.67 (s, 4H), 1.99 (q, J=7.0, 6.4 Hz, 3H), 1.66 (s, 2H), 1.45 (s, 2H), 1.14 (s, 1H), 0.84 (d, J=7.1 Hz, 3H). Mass (m/z): 320.6 [M+H]⁺.

### Compound 331

### 1,3-dimethyl-5-((1-methyl-1H-benzo[d]imidazol-5-yl)amino)-1,3-dihydro-2H-benzo[d]imidazol-2-one (847)

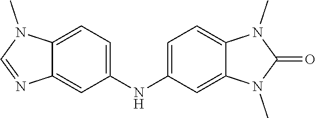 [SMILES: Cn1cnc2cc(Nc3ccc4c(c3)n(C)c(=O)n4C)ccc21]

The title compound 331 (8.3 mg) was prepared in a yield of 11.4% as a white powder from 5-amino-1,3-dimethyl-1,3-dihydro-2H-benzo[d]imidazol-2-one (50 mg, 0.24 mmol) and 5-bromo-1-methyl-1H-benzo[d]imidazole (42 mg, 0.24 mmol) according to the procedure for compound 1. ¹H NMR (400 MHz, DMSO-d₆) δ 8.06 (s, 1H), 7.81 (s, 1H), 7.42 (d, J=8.6 Hz, 1H), 7.27 (s, 1H), 7.07-6.96 (m, 2H), 6.81 (d, J=1.9 Hz, 1H), 6.76 (d, J=8.3 Hz, 1H), 3.79 (s, 3H), 3.29 (s, 3H), 3.25 (s, 3H). Mass (m/z): 308.4 [M+H]⁺.

### Compound 332

### 5-((4-(4-hydroxy-4-(trifluoromethyl)piperidin-1-yl)phenyl)amino)-1,3-dimethyl-1,3-dihydro-2H-benzo[d]imidazol-2-one

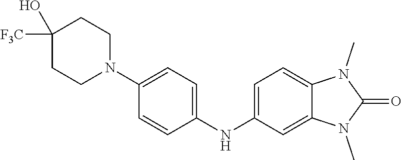 [SMILES: Cn1c(=O)n(C)c2cc(Nc3ccc(N4CCC(O)(C(F)(F)F)CC4)cc3)ccc21]

The title compound 332 (5.6 mg) was prepared in a yield of 6.42% as a grey powder from 1-(4-aminophenyl)-4-(trifluoromethyl)piperidin-4-ol (50 mg, 0.19 mmol) and 5-bromo-1,3-dimethyl-1,3-dihydro-2H-benzo[d]imidazol-2-one (54 mg, 0.21 mmol) according to the procedure for compound 1. ¹H NMR (301 MHz, DMSO-d₆) δ 7.64 (s, 1H), 7.01-6.83 (m, 5H), 6.75 (d, J=2.0 Hz, 1H), 6.72-6.64 (m, 1H), 5.92 (s, 1H), 3.26 (dd, J=7.2, 1.5 Hz, 6H), 2.85 (t, J=11.5 Hz, 3H), 1.88-1.61 (m, 5H). Mass (m/z): 421.6 [M+H]⁺.

### Compound 333

### 6-((4-(4-hydroxy-4-(trifluoromethyl)piperidin-1-yl)phenyl)amino)-3-methylbenzo[d]oxazol-2(3H)-one

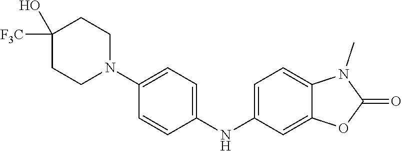 [SMILES: Cn1c(=O)oc2cc(Nc3ccc(N4CCC(O)(C(F)(F)F)CC4)cc3)ccc21]

The title compound 333 (2.4 mg) was prepared in a yield of 3.07% as a pale yellow powder from 1-(4-aminophenyl)-4-(trifluoromethyl)piperidin-4-ol (50 mg, 0.19 mmol) and 6-bromo-3-methylbenzo[d]oxazol-2(3H)-one (48 mg, 0.21 mmol) according to the procedure for compound 1. ¹H NMR (DMSO-d₆) δ 7.81 (s, 1H), 7.21 (s, 1H), 7.05 (d, J=8.4 Hz, 1H), 7.00-6.86 (m, 3H), 6.78 (d, J=9.0 Hz, 1H), 6.66 (s, 1H), 5.93 (s, 1H), 2.88 (d, J=11.3 Hz, 2H), 2.00 (q, J=7.0, 6.3 Hz, 3H), 1.73 (s, 2H), 1.45 (s, 2H), 0.84 (d, J=7.0 Hz, 2H). Mass (m/z): 408.6 [M+H]⁺.

### Compound 334

### 3-(2-methoxyethyl)-1-methyl-5-((4-(4-(trifluoromethyl)piperidin-1-yl) phenyl)amino)-1,3-dihydro-2H-benzo[d]imidazol-2-one

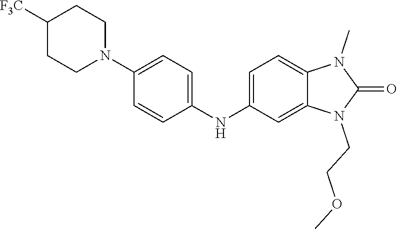 [SMILES: COCCn1c(=O)n(C)c2ccc(Nc3ccc(N4CCC(C(F)(F)F)CC4)cc3)cc21]

The title compound 334 (44.0 mg) was prepared in a total yield of 29.5% as a dark gray powder from 4-(4-(trifluoromethyl)piperidin-1-yl)aniline (79.3 mg, 0.33 mmol), 5-bromo-3-(2-methoxyethyl)-1-methyl-1,3-dihydro-2H-benzo[d]imidazol-2-one (71 mg, 0.25 mmol), Pd₂(dba)₃ (2.3 mg, 2.5 umol), X-Phos (6.0 mg, 12.5 umol), and t-BuONa (36.8 mg, 0.38 mmol) according to the procedure for compound 1. ¹H NMR (400 MHz, Methanol-d₄) δ 7.06-6.91 (m, 6H), 6.84-6.78 (m, 1H), 4.01 (t, J=5.3 Hz, 2H), 3.66 (t, J=5.3 Hz, 3H), 3.63-3.55 (m, 2H), 3.39 (s, 3H), 3.33 (s, 3H), 2.83-2.53 (m, 2H), 2.36-2.22 (m, 2H), 2.01-1.92 (m, 2H), 1.74 (qd, J=12.5, 4.1 Hz, 3H). Mass (m/z): 449.3 [M+H]⁺.

### Compound 335

### 1,3,3-trimethyl-6-((4-(4-(trifluoromethyl)piperidin-1-yl)phenyl)amino) indolin-2-one

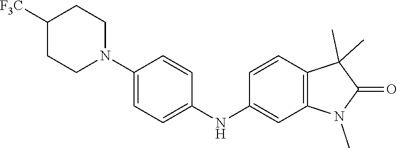 [SMILES: CN1C(=O)C(C)(C)c2ccc(Nc3ccc(N4CCC(C(F)(F)F)CC4)cc3)cc21]

The title compound 335 (51.1 mg) was prepared in a total yield of 36.8% as a light gray powder from 4-(4-(trifluoromethyl)piperidin-1-yl)aniline (122 mg, 0.50 mmol), 6-bromo-1,3,3-trimethylindolin-2-one (84 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 8.09-5.93 (m, 7H), 3.16 (s, 3H), 2.53-2.31 (m, 1H), 2.21-1.66 (m, 4H), 1.31 (s, 6H). Mass (m/z): 418.3 [M+H]⁺.

### Compound 336

### 1-methyl-N-(4-(4-(trifluoromethyl)piperidin-1-yl)phenyl)-1H-benzo[d]imidazol-5-amine

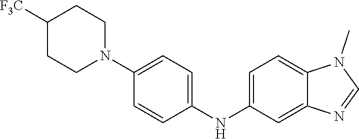 [SMILES: Cn1cnc2cc(Nc3ccc(N4CCC(C(F)(F)F)CC4)cc3)ccc21]

The title compound 336 (35.1 mg) was prepared in a total yield of 28.1% as a yellow powder from 4-(4-(trifluoromethyl)piperidin-1-yl)aniline (105 mg, 0.43 mmol), 5-bromo-1-methyl-1H-benzo[d]imidazole (70 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 8.02 (s, 1H), 7.40 (d, J=8.7 Hz, 1H), 7.32-7.20 (m, 1H), 7.13-6.75 (m, 5H), 3.85 (s, 3H), 3.69-3.49 (m, 2H), 2.79-2.48 (m, 2H), 2.35-2.16 (m, 2H), 2.01-1.87 (m, 2H), 1.80-1.65 (m, 2H). Mass (m/z): 375.3 [M+H]⁺.

### Compound 337

### N-(4-(4-(trifluoromethyl)piperidin-1-yl)phenyl)benzofuran-5-amine

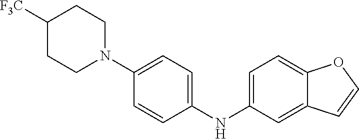 [SMILES: FC(F)(F)C1CCN(c2ccc(Nc3ccc4occc4c3)cc2)CC1]

The title compound 337 (54.6 mg) was prepared in a total yield of 45.5% as a yellow powder from 4-(4-(trifluoromethyl)piperidin-1-yl)aniline (105 mg, 0.43 mmol), 5-bromobenzofuran (66 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 7.64 (d, J=2.2 Hz, 1H), 7.47-6.75 (m, 7H), 6.68 (d, J=1.6 Hz, 1H), 3.86-3.40 (m, 2H), 3.04-2.16 (m, 3H), 2.04-1.91 (m, 2H), 1.81-1.61 (m, 2H). Mass (m/z): 361.2 [M+H]⁺.

### Compound 338

### 1,3,3-trimethyl-N-(4-(4-methylpiperidin-1-yl)phenyl)indolin-6-amine

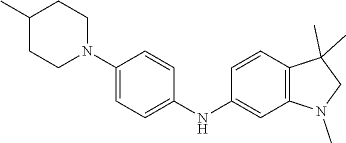

The title compound 338 (43.2 mg) was prepared in a total yield of 45.5% as a yellow powder from 4-(4-methylpiperidin-1-yl)aniline (82 mg, 0.43 mmol), 6-bromo-1,3,3-trimethylindoline (80 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 6.92-6.86 (m, 2H), 6.85-6.80 (m, 2H), 6.69 (s, 1H), 6.09 (s, 1H), 3.38-3.31 (m, 2H), 2.90 (s, 2H), 2.56 (s, 3H), 2.54-2.44 (m, 2H), 1.70-1.59 (m, 2H), 1.42-1.21 (m, 3H), 1.14 (s, 6H), 0.88 (d, J=6.3 Hz, 3H). Mass (m/z): 350.3 [M+H]⁺.

### Compound 339

### 2-methyl-N-(4-(4-methylpiperidin-1-yl)phenyl)benzo[d]oxazol-6-amine

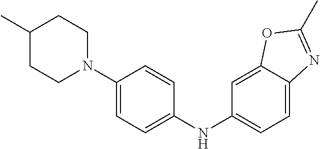

The title compound 339 (69.4 mg) was prepared in a total yield of 84.3% as a white solid from 4-(4-methylpiperidin-1-yl)aniline (54 mg, 0.283 mmol), 6-bromo-2-methylbenzo[d]oxazole (50 mg, 0.256 mmol), Pd₂(dba)₃ (2 mg, 0.002 mmol), X-phos (6 mg, 0.01 mmol), Cs₂CO₃ (126 mg, 0.384 mmol) and 1,4-dioxane (5 mL) according to the procedure for compound 1. ¹H NMR (400 MHz, DMSO-d₆) δ 8.05 (s, 1H), 7.40 (d, J=8.4 Hz, 1H), 7.11-6.83 (m, 6H), 3.52 (s, 2H), 3.36 (s, 3H), 2.55 (s, 2H), 1.68 (s, 2H), 1.46 (s, 1H), 1.24 (d, J=9.2 Hz, 2H), 0.94 (d, J=6.4 Hz, 3H). Mass (m/z): 322.3 [M+H]⁺.

### Compound 340

### 1,3-dimethyl-2-(methylimino)-N-(4-(4-(trifluoromethyl)piperidin-1-yl) phenyl)-2,3-dihydro-1H-benzo[d]imidazol-5-amine

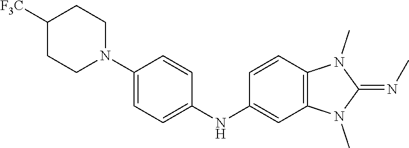 [SMILES: C/N=c1\n(C)c2ccc(Nc3ccc(N4CCC(C(F)(F)F)CC4)cc3)cc2n1C]

The title compound 340 (2.2 mg) was prepared in a total yield of 2.7% as a white solid from 4-(4-(trifluoromethyl)piperidin-1-yl)aniline (57 mg, 0.236 mmol), 5-bromo-N,1,3-trimethyl-1,3-dihydro-2H-benzo[d]imidazol-2-imine (50 mg, 0.197 mmol), Pd₂(dba)₃ (2 mg, 0.002 mmol), X-phos (6 mg, 0.01 mmol), Cs₂CO₃ (97 mg, 0.296 mmol) and 1,4-dioxane (5 mL) according to the procedure for compound 1. ¹H NMR (400 MHz, DMSO-d₆) δ 6.89-6.79 (m, 5H), 6.68 (d, J=2.0 Hz, 1H), 6.62 (dd, J=8.4, 2.0 Hz, 1H), 3.21 (s, 3H), 3.18 (s, 3H), 3.12 (d, J=10.0 Hz, 2H), 2.91 (td, J=8.4, 7.6, 4.0 Hz, 3H), 1.92 (dd, J=10.4, 5.2 Hz, 2H), 1.75 (d, J=10.4 Hz, 2H), 1.02 (d, J=6.0 Hz, 3H). Mass (m/z): 418.3 [M+H]⁺.

### Compound 341

### 5-((4-(4-hydroxy-4-methylpiperidin-1-yl)phenyl)amino)-1,3-dimethyl-1,3-dihydro-2H-benzo[d]imidazol-2-one

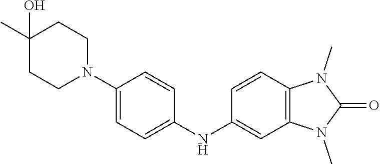 [SMILES: Cn1c(=O)n(C)c2cc(Nc3ccc(N4CCC(C)(O)CC4)cc3)ccc21]

The title compound 341 (12.1 mg) was prepared in a total yield of 15.9% as a green solid from 1-(4-aminophenyl)-4-methylpiperidin-4-ol (51 mg, 0.250 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, DMSO-d₆) δ 7.65-7.59 (m, 1H), 6.98-6.91 (m, 3H), 6.87-6.82 (m, 2H), 6.73 (d, J=2.0 Hz, 1H), 6.68 (dd, J=8.4, 2.0 Hz, 1H), 4.27 (q, J=1.6 Hz, 1H), 3.26 (d, J=10.4 Hz, 6H), 3.10 (dt, J=12.0, 4.4 Hz, 2H), 3.01 (dt, J=12.4, 6.4 Hz, 2H), 1.57 (dd, J=6.4, 4.4 Hz, 4H), 1.15 (s, 3H). Mass (m/z): 367.3 [M+H]⁺.

### Compound 342

### 1,3-dimethyl-5-(thiophen-3-ylamino)-1,3-dihydro-2H-benzo[d]imidazol-2-one

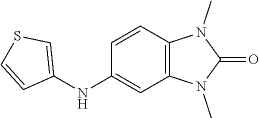 [SMILES: Cn1c(=O)n(C)c2cc(Nc3ccsc3)ccc21]

The title compound 342 (6.4 mg) was prepared in a total yield of 11.9% as a brown solid from thiophen-3-amine (34 mg, 0.250 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, DMSO-d6) δ 7.93 (d, J=2.8 Hz, 1H), 7.82 (s, 1H), 7.45 (dd, J=8.8, 2.8 Hz, 1H), 6.99 (d, J=8.4 Hz, 1H), 6.78-6.72 (m, 2H), 6.69 (dd, J=8.4, 2.0 Hz, 1H), 3.27 (d, J=7.6 Hz, 6H). Mass (m/z): 260.3 [M+H]⁺.

### Compound 343

### 1,3-dimethyl-6-((4-(4-(trifluoromethyl)piperidin-1-yl)phenyl)amino)-1,3-dihydro-2H-imidazo[4,5-b]pyridin-2-one

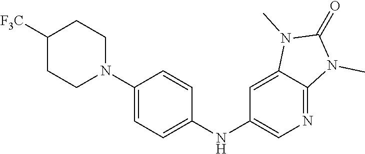 [SMILES: Cn1c(=O)n(C)c2ncc(Nc3ccc(N4CCC(C(F)(F)F)CC4)cc3)cc21]

The title compound 343 (7.0 mg) was prepared in a total yield of 8.3% as a green solid from 4-(4-(trifluoromethyl)piperidin-1-yl)aniline (60 mg, 0.248 mmol), 5-bromo-1,3-dimethyl-1,3-dihydro-2H-benzo[d]imidazol-2-one (50 mg, 0.207 mmol), Pd₂(dba)₃ (2 mg, 0.002 mmol), X-phos (6 mg, 0.01 mmol), Cs₂CO₃ (102 mg, 0.310 mmol) and 1,4-dioxane (5 mL) according to the procedure for compound 1. ¹H NMR (400 MHz, DMSO-d₆) δ 7.72 (s, 2H), 7.25-6.80 (m, 5H), 3.47 (s, 3H), 3.30 (s, 6H), 2.73 (s, 1H), 1.91 (s, 2H), 1.63 (s, 2H). Mass (m/z): 406.3 [M+H]⁺.

### Compound 344

### 6-((1,3-dimethyl-2-oxo-2,3-dihydro-1H-benzo[d]imidazol-5-yl)amino)-3-methylbenzo[d]oxazol-2(3H)-one

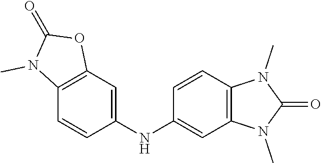 [SMILES: Cn1c(=O)oc2cc(Nc3ccc4c(c3)n(C)c(=O)n4C)ccc21]

The title compound 344 (11.3 mg) was prepared in a total yield of 16.8% as a white solid from 6-amino-3-methylbenzo[d]oxazol-2(3H)-one (41 mg, 0.250 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, DMSO-d₆) δ 7.89 (s, 1H), 7.01 (d, J=8.4 Hz, 1H), 6.96 (d, J=8.4 Hz, 1H), 6.90 (d, J=2.0 Hz, 1H), 6.78 (dd, J=8.0, 2.1 Hz, 2H), 6.70 (dd, J=8.0, 2.0 Hz, 1H), 3.25-3.22 (m, 6H), 3.20 (s, 3H). Mass (m/z): 325.3 [M+H]⁺.

### Compound 345

### 2-imino-1,3-dimethyl-N-(4-(4-methylpiperidin-1-yl)phenyl)-2,3-dihydro-1H-benzo[d]imidazol-5-amine

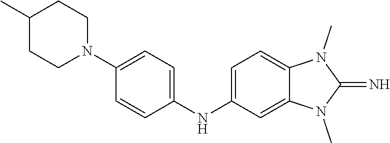 [SMILES: CC1CCN(c2ccc(Nc3ccc4c(c3)n(C)c(=N)n4C)cc2)CC1]

The title compound 345 (13.9 mg) was prepared in a total yield of 79.2% as a light blue solid from 4-(4-methylpiperidin-1-yl)aniline (12.4 mg, 0.065 mmol), 5-bromo-1,3-dimethyl-1,3-dihydro-2H-benzo[d]imidazol-2-imine (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.47-6.79 (m, 7H), 3.78-3.67 (m, 2H), 3.64 (s, 3H), 3.59 (s, 3H), 2.83-2.54 (m, 2H), 1.97-1.68 (m, 2H), 1.53 (m, 1H), 1.47-1.36 (m, 2H), 1.01 (d, J=6.4 Hz, 3H). Mass (m/z): 350.2 [M+H]⁺.

### Compound 346

### 4-methyl-8-((4-(4-(trifluoromethyl)piperidin-1-yl)phenyl)amino)-3,4-dihydrobenzo[f][1,4]oxazepin-5(2H)-one

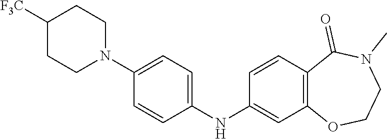 [SMILES: CN1CCOc2cc(Nc3ccc(N4CCC(C(F)(F)F)CC4)cc3)ccc2C1=O]

The title compound 346 (18.3 mg) was prepared in a total yield of 87.1% as a wheat solid from 4-(4-(trifluoromethyl)piperidin-1-yl)aniline (15.9 mg, 0.065 mmol), 8-bromo-4-methyl-3,4-dihydrobenzo[f][1,4]oxazepin-5(2H)-one (12.8 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Chloroform-d) δ 7.74 (d, J=8.4 Hz, 1H), 7.08 (d, J=8.4 Hz, 2H), 6.91 (d, J=8.4 Hz, 2H), 6.59 (dd, J=8.4, 2.4 Hz, 1H), 6.43 (d, J=2.4 Hz, 1H), 4.33 (t, J=4.8 Hz, 2H), 3.74-3.63 (m, 2H), 3.53 (t, J=4.8 Hz, 2H), 3.17 (s, 3H), 2.81-2.57 (m, 2H), 2.15 (m, 1H), 2.02-1.92 (m, 2H), 1.85-1.68 (m, 2H). Mass (m/z): 420.3 [M+H]⁺.

### Compound 347

### 1-methyl-N-(4-(4-(trifluoromethyl)piperidin-1-yl)phenyl)-1H-indol-5-amine

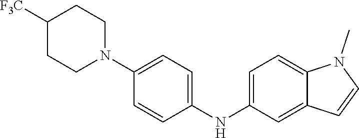 [SMILES: Cn1ccc2cc(Nc3ccc(N4CCC(C(F)(F)F)CC4)cc3)ccc21]

The title compound 347 (23.9 mg) was prepared in a yield of 31.27% as a pale gray powder from 4-(4-(trifluoromethyl)piperidin-1-yl)aniline (50 mg, 0.20 mmol) and 5-bromo-1-methyl-1H-indole (47 mg, 0.23 mmol) according to the procedure for compound 1. ¹H NMR (DMSO-d₆) δ 7.57-6.42 (br, 9H), 4.62-3.21 (br, 8H), 2.64-1.41 (br, 4H). Mass (m/z): 374.2 [M+H]⁺.

### Compound 348

### 1-methyl-N-(4-(4-(trifluoromethyl)piperidin-1-yl)phenyl)-1H-indol-6-amine

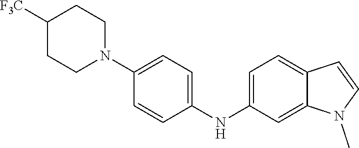 [SMILES: Cn1ccc2ccc(Nc3ccc(N4CCC(C(F)(F)F)CC4)cc3)cc21]

The title compound 348 (38.0 mg) was prepared in a yield of 49.71% as a white powder from 4-(4-(trifluoromethyl)piperidin-1-yl)aniline (50 mg, 0.20 mmol) and 6-bromo-1-methyl-1H-indole (47 mg, 0.22 mmol) according to the procedure for compound 1. ¹H NMR (DMSO-d₆) δ 7.86-6.63 (br, 9H), 4.51-3.04 (br, 8H), 2.36-1.52 (br, 4H). Mass (m/z): 374.1 [M+H]⁺.

### Compound 349

### 3-methyl-5-((4-(4-(trifluoromethyl)piperidin-1-yl)phenyl)amino)benzo[d]oxazol-2(3)-one

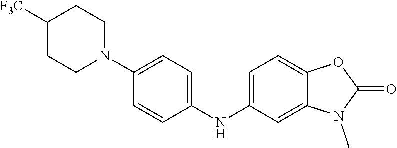 [SMILES: Cn1c(=O)oc2ccc(Nc3ccc(N4CCC(C(F)(F)F)CC4)cc3)cc21]

The title compound 349 (8.8 mg) was prepared in a yield of 10.25% as a dim gray powder from 4-(4-(trifluoromethyl)piperidin-1-yl)aniline (50 mg, 0.22 mmol) and 5-bromo-3-methylbenzo[d]oxazol-2(3H)-one (59 mg, 0.22 mmol) according to the procedure for compound 1. ¹H NMR (400 MHz, DMSO-d₆) δ 7.84 (s, 1H), 7.12 (d, J=8.6 Hz, 1H), 7.03-6.95 (m, 2H), 6.94-6.87 (m, 2H), 6.75 (d, J=2.1 Hz, 1H), 6.63 (dt, J=8.6, 1.9 Hz, 1H), 3.62 (d, J=12.4 Hz, 2H), 3.26 (s, 3H), 2.63 (td, J=12.3, 2.5 Hz, 2H), 1.93-1.79 (m, 2H), 1.57 (qd, J=12.4, 4.2 Hz, 3H). Mass (m/z): 392.6 [M+H]⁺.

### Compound 350

### 1-methyl-N-(4-(4-(trifluoromethyl)piperidin-1-yl)phenyl)-1H-benzo[d]imidazol-6-amine

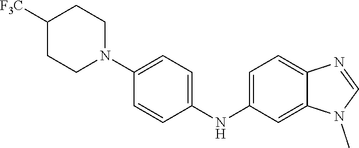 [SMILES: Cn1cnc2ccc(Nc3ccc(N4CCC(C(F)(F)F)CC4)cc3)cc21]

The title compound 350 (35.6 mg) was prepared in a total yield of 28.5% as a yellow powder from 4-(4-(trifluoromethyl)piperidin-1-yl)aniline (82 mg, 0.43 mmol), 6-bromo-1-methyl-1H-benzo[d]imidazole (70 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, DMSO-d₆) δ 8.26 (s, 1H), 7.96 (s, 1H), 7.60 (d, J=8.7 Hz, 1H), 7.09-7.02 (m, 3H), 6.97-6.88 (m, 3H), 3.79 (d, J=1.6 Hz, 3H), 3.67-3.60 (m, 2H), 2.64 (td, J=12.4, 2.5 Hz, 2H), 2.47-2.40 (m, 1H), 1.93-1.84 (m, 2H), 1.58 (qd, J=12.5, 4.1 Hz, 2H). Mass (m/z): 375.3 [M+H]⁺.

### Compound 351

### N-(4-(4-(trifluoromethyl)piperidin-1-yl)phenyl)benzofuran-6-amine

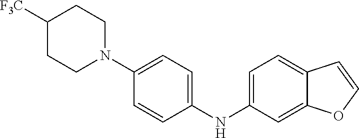 [SMILES: FC(F)(F)C1CCN(c2ccc(Nc3ccc4ccoc4c3)cc2)CC1]

The title compound 351 (10.3 mg) was prepared in a total yield of 8.6% as a yellow powder from 4-(4-(trifluoromethyl)piperidin-1-yl)aniline (105 mg, 0.43 mmol), 6-bromobenzofuran (66 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 7.55 (d, J=2.2 Hz, 1H), 7.39 (d, J=8.4 Hz, 1H), 7.09 (d, J=8.9 Hz, 3H), 6.99 (d, J=8.4 Hz, 2H), 6.91 (d, J=8.4 Hz, 1H), 6.70 (dd, J=2.2, 1.0 Hz, 1H), 3.67-3.54 (m, 2H), 2.78-2.59 (m, 2H), 2.38-2.23 (m, 2H), 2.03-1.91 (m, 2H), 1.75 (qd, J=12.6, 4.1 Hz, 2H). Mass (m/z): 361.2 [M+H]⁺.

### Compound 352

### 1-methyl-N5-(4-(4-methylpiperidin-1-yl)phenyl)-1H-indazole-3,5-diamine

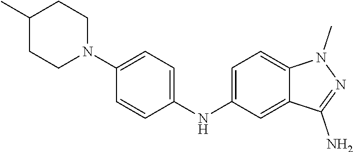 [SMILES: CC1CCN(c2ccc(Nc3ccc4c(c3)c(N)nn4C)cc2)CC1]

The title compound 352 (9.9 mg) was prepared in a total yield of 8.9% as a yellow powder from 4-(4-methylpiperidin-1-yl)aniline (81.7 mg, 0.43 mmol), 5-bromo-1-methyl-1H-indazol-3-amine (75 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 7.38-6.52 (m, 7H), 3.76 (s, 3H), 3.61-3.35 (m, 2H), 2.98-2.43 (m, 2H), 1.87-1.71 (m, 2H), 1.58-1.35 (m, 3H), 1.01 (d, J=6.2 Hz, 3H). Mass (m/z): 336.4 [M+H]⁺.

### Compound 353

### 1-methyl-5-((4-(4-methylpiperidin-1-yl)phenyl)amino)-1H-indole-3-carboxylic acid

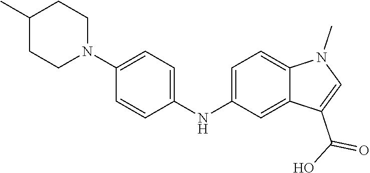 [SMILES: CC1CCN(c2ccc(Nc3ccc4c(c3)c(C(=O)O)cn4C)cc2)CC1]

The title compound 353 (11.1 mg) was prepared in a total yield of 9.2% as a yellow powder from 4-(4-methylpiperidin-1-yl)aniline (81.7 mg, 0.43 mmol), 5-bromo-1-methyl-1H-indole-3-carboxylic acid (85 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 8.18-6.47 (m, 8H), 4.17-3.61 (m, 7H), 1.75-1.45 (m, 5H), 1.02 (d, J=6.1 Hz, 3H). Mass (m/z): 364.3 [M+H]⁺.

### Compound 354

### 1-(2-methoxyethyl)-3-methyl-5-((4-(4-(trifluoromethyl)piperidin-1-yl) phenyl)amino)-1,3-dihydro-2H-benzo[d]imidazol-2-one

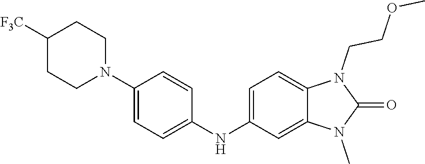 [SMILES: COCCn1c(=O)n(C)c2cc(Nc3ccc(N4CCC(C(F)(F)F)CC4)cc3)ccc21]

The title compound 354 (59.1 mg) was prepared in a total yield of 40.0% as a gray powder from 4-(4-(trifluoromethyl)piperidin-1-yl)aniline (105 mg, 0.43 mmol), 5-bromo-1-(2-methoxyethyl)-3-methyl-1,3-dihydro-2H-benzo[d]imidazol-2-one (94.7 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 8.10-6.03 (m, 7H), 4.03 (t, J=5.4 Hz, 2H), 3.87-3.42 (m, 4H), 3.33-3.31 (m, 5H), 2.34-2.21 (m, 1H), 2.05-1.91 (m, 2H), 1.80-1.65 (m, 2H). Mass (m/z): 449.3 [M+H]⁺.

### Compound 355

### 1-(2-(dimethylamino) ethyl)-3-methyl-5-((4-(4-(trifluoromethyl)piperidin-1-yl)phenyl)amino)-1,3-dihydro-2H-benzo[d]imidazol-2-one

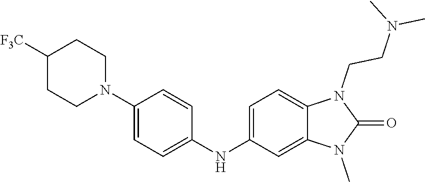 [SMILES: CN(C)CCn1c(=O)n(C)c2cc(Nc3ccc(N4CCC(C(F)(F)F)CC4)cc3)ccc21]

The title compound 355 (7.0 mg) was prepared in a total yield of 4.6% as a gray powder from 4-(4-(trifluoromethyl)piperidin-1-yl)aniline (80 mg, 0.43 mmol), 5-bromo-1-(2-(dimethylamino)ethyl)-3-methyl-1,3-dihydro-2H-benzo[d]imidazol-2-one (74.5 mg, 0.25 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 7.27-6.47 (m, 7H), 3.99 (t, J=6.5 Hz, 2H), 3.64-3.36 (m, 2H), 3.24 (s, 3H), 2.87 (t, J=6.5 Hz, 2H), 2.76-2.46 (m, 2H), 2.45 (s, 6H), 2.24-2.12 (m, 1H), 1.94-1.82 (m, 2H), 1.62 (q, J=12.5, 11.9 Hz, 2H). Mass (m/z): 462.4 [M+H]⁺.

### Compound 356

### N-(1-methyl-5-((4-(4-methylpiperidin-1-yl)phenyl)amino)-1H-indazol-3-yl) acetamide

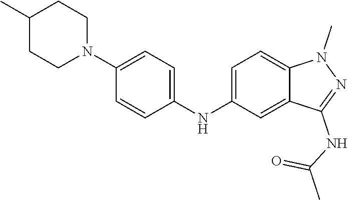 [SMILES: CC(=O)Nc1nn(C)c2ccc(Nc3ccc(N4CCC(C)CC4)cc3)cc12]

The title compound 356 (4.0 mg) was prepared in a total yield of 3.2% as a gray powder from 4-(4-methylpiperidin-1-yl)aniline (82 mg, 0.43 mmol), N-(5-bromo-1-methyl-1H-indazol-3-yl)acetamide (74.5 mg, 0.25 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 7.84-6.71 (m, 7H), 4.24-3.43 (m, 7H), 2.20 (s, 3H), 2.07-2.00 (m, 1H), 1.93-1.40 (m, 7H), 1.06 (d, J=6.0 Hz, 4H). Mass (m/z): 378.3 [M+H]⁺.

### Compound 357

### N-(4-(4-(trifluoromethyl)piperidin-1-yl)phenyl)-1H-benzo[d]imidazol-6-amine

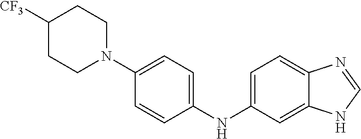 [SMILES: FC(F)(F)C1CCN(c2ccc(Nc3ccc4nc[nH]c4c3)cc2)CC1]

To a solution of tert-butyl 6-((4-(4-(trifluoromethyl)piperidin-1-yl)phenyl) amino)-1H-benzo[d]imidazole-1-carboxylate (153 mg, 0.33 mmol) in EtOH (5 mL) was added a solution of HCl in 1,4-dioxane (5 mL). Then the solution was stirred for 30 mins at r.t. and concentrated. 10 ml water was added. The PH of the filtration was adjusted to 8-9 with sodium carbonate solution. Then the mixture was extracted by DCM (15 mL×3). The combined organic layers were washed with water (10 mL), dried over Na₂SO₄ and concentrated. The residue was purified by perp-TLC (MeOH/DCM=1/10) to afford the desired product as a light gray solid. (6.1 mg, 5.0%). ¹H NMR (400 MHz, Methanol-d₄) δ 8.56 (s, 1H), 8.04-6.31 (m, 7H), 4.01-3.51 (m, 2H), 3.11-2.57 (m, 2H), 2.41-2.27 (m, 1H), 2.13-1.65 (m, 4H). Mass (m/z): 361.2 [M+H]⁺.

### Compound 358

### N-(4-(4-(trifluoromethyl)piperidin-1-yl)phenyl)benzo[d]isoxazol-6-amine

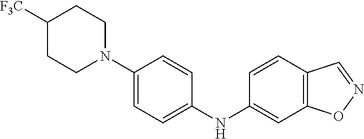 [SMILES: FC(F)(F)C1CCN(c2ccc(Nc3ccc4cnoc4c3)cc2)CC1]

The title compound 358 (19.1 mg) was prepared in a total yield of 21.0% as a white solid from 4-(4-(trifluoromethyl)piperidin-1-yl)aniline (73 mg, 0.303 mmol), 6-bromobenzo[d]isoxazole (50 mg, 0.252 mmol), Pd₂(dba)₃ (2 mg, 0.002 mmol), X-phos (6 mg, 0.01 mmol), Cs₂CO₃ (124 mg, 0.374 mmol) and 1,4-dioxane (5 mL) according to the procedure for compound 1. ¹H NMR (400 MHz, DMSO-d₆) δ 10.64 (s, 1H), 8.46 (s, 1H), 7.25 (d, J=8.4 Hz, 1H), 7.06-7.00 (m, 2H), 6.95 (d, J=8.8 Hz, 2H), 6.45 (d, J=2.0 Hz, 1H), 6.34 (dd, J=8.4, 2.1 Hz, 1H), 3.71 (d, J=12.0 Hz, 2H), 2.67 (td, J=12.4, 2.4 Hz, 2H), 2.44 (ddd, J=15.2, 7.6, 3.6 Hz, 1H), 1.94-1.84 (m, 2H), 1.57 (qd, J=12.4, 4.0 Hz, 2H). Mass (m/z): 362.3 [M+H]⁺.

### Compound 359

### N-(4-(4-(trifluoromethyl)piperidin-1-yl)phenyl)benzo[d]oxazol-5-amine

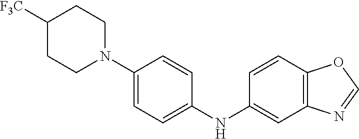 [SMILES: FC(F)(F)C1CCN(c2ccc(Nc3ccc4ocnc4c3)cc2)CC1]

The title compound 359 (9.3 mg) was prepared in a total yield of 10.2% as a white solid from 4-(4-(trifluoromethyl)piperidin-1-yl)aniline (73 mg, 0.303 mmol), 5-bromobenzo[d]oxazole (50 mg, 0.252 mmol), Pd₂(dba)₃ (2 mg, 0.002 mmol), X-phos (6 mg, 0.01 mmol), Cs₂CO₃ (124 mg, 0.374 mmol) and 1,4-dioxane (5 mL) according to the procedure for compound 1. ¹H NMR (400 MHz, DMSO-d₆) δ 8.59 (s, 1H), 7.90 (s, 1H), 7.56 (d, J=8.8 Hz, 1H), 7.23 (d, J=2.0 Hz, 1H), 7.04-6.99 (m, 3H), 6.96-6.90 (m, 2H), 3.63 (d, J=12.4 Hz, 2H), 2.63 (td, J=12.4, 2.4 Hz, 2H), 2.43 (dtd, J=12.4, 8.4, 3.6 Hz, 1H), 1.93-1.84 (m, 2H), 1.58 (qd, J=12.4, 4.0 Hz, 2H). Mass (m/z): 362.3 [M+H]⁺.

### Compound 360

### N-(4-(4-(trifluoromethyl)piperidin-1-yl)phenyl)benzo[d]oxazol-6-amine

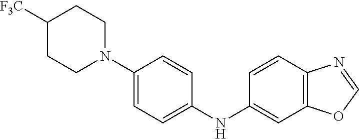 [SMILES: FC(F)(F)C1CCN(c2ccc(Nc3ccc4ncoc4c3)cc2)CC1]

The title compound 360 (27.3 mg) was prepared in a total yield of 30.0% as a white solid from 4-(4-(trifluoromethyl)piperidin-1-yl)aniline (73 mg, 0.303 mmol), 6-bromobenzo[d]oxazole (50 mg, 0.252 mmol), Pd₂(dba)₃ (2 mg, 0.002 mmol), X-phos (6 mg, 0.01 mmol) and Cs₂CO₃ (124 mg, 0.374 mmol) and 1,4-dioxane (5 mL) according to the procedure for compound 1. ¹H NMR (400 MHz, DMSO-d₆) δ 8.38 (s, 1H), 8.08 (s, 1H), 7.48 (d, J=8.8 Hz, 1H), 7.09 (d, J=2.0 Hz, 1H), 7.04-6.96 (m, 2H), 6.88 (td, J=8.8, 2.0 Hz, 3H), 3.59 (d, J=11.6 Hz, 2H), 2.57 (td, J=12.4, 2.4 Hz, 2H), 2.36 (dq, J=12.4, 3.6 Hz, 1H), 1.88-1.76 (m, 2H), 1.51 (qd, J=12.4, 4.0 Hz, 2H). Mass (m/z): 362.3 [M+H]⁺.

### Compound 361

### methyl 3-((4-((1,3-dimethyl-2-oxo-2,3-dihydro-1H-benzo[d]imidazol-5-yl) amino)phenyl)(ethyl)amino)propanoate

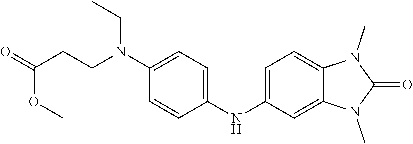 [SMILES: CCN(CCC(=O)OC)c1ccc(Nc2ccc3c(c2)n(C)c(=O)n3C)cc1]

The title compound 361 (10.3 mg) was prepared in a total yield of 53.9% as a white solid from methyl 3-((4-aminophenyl)(ethyl)amino)propanoate (14.4 mg, 0.065 mmol), 5-bromo-1,3-dimethyl-1,3-dihydro-2H-benzo[d]imidazol-2-one (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.35-6.70 (m, 7H), 3.67 (s, 3H), 3.62 (s, 3H), 3.41-3.32 (m, 4H), 3.29 (s, 3H), 2.58-2.46 (m, 2H), 1.08 (t, J=6.8 Hz, 3H). Mass (m/z): 383.2 [M+H]⁺.

### Compound 362

### 1,3-dimethyl-5-((6-(4-(trifluoromethyl)piperidin-1-yl)pyridin-3-yl)amino)-1,3-dihydro-2H-benzo[d]imidazol-2-one

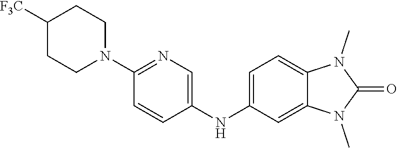 [SMILES: Cn1c(=O)n(C)c2cc(Nc3ccc(N4CCC(C(F)(F)F)CC4)nc3)ccc21]

The title compound 362 (13.7 mg) was prepared in a total yield of 67.6% as a yellow solid from 6-(4-(trifluoromethyl)piperidin-1-yl)pyridin-3-amine (15.9 mg, 0.065 mmol), 5-bromo-2-methylisoindoline-1,3-dione (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.96 (d, J=2.4 Hz, 1H), 7.43 (dd, J=8.8, 2.4 Hz, 1H), 6.97 (d, J=8.8 Hz, 1H), 6.84 (d, J=8.8 Hz, 1H), 6.77-6.70 (m, 2H), 4.25-4.17 (m, 2H), 3.37 (s, 3H), 3.34 (s, 3H), 2.86-2.73 (m, 2H), 2.38 (m, 1H), 1.99-1.88 (m, 2H), 1.67-1.53 (m, 2H). Mass (m/z): 406.2 [M+H]⁺.

### Compound 363

### 1-methyl-5-((4-(4-(trifluoromethyl)piperidin-1-yl)phenyl)amino)-1,3-dihydro-2H-benzo[d]imidazol-2-one

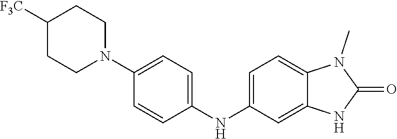 [SMILES: Cn1c(=O)[nH]c2cc(Nc3ccc(N4CCC(C(F)(F)F)CC4)cc3)ccc21]

The title compound 363 (16.5 mg) was prepared in a total yield of 84.4% as a yellow solid from 4-(4-(trifluoromethyl)piperidin-1-yl)aniline (15.9 mg, 0.065 mmol), 5-bromo-1-methyl-1,3-dihydro-2H-benzo[d]imidazol-2-one (11.3 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.46-6.65 (m, 7H), 3.71-3.65 (m, 2H), 3.58 (s, 3H), 2.78-2.66 (m, 2H), 2.13 (m, 1H), 2.01-1.87 (m, 2H), 1.76-1.67 (m, 2H). Mass (m/z): 391.2 [M+H]⁺.

### Compound 364

### 1-methyl-N-(4-(4-(trifluoromethyl)piperidin-1-yl)phenyl)-1H-indazol

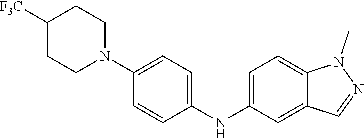 [SMILES: Cn1ncc2cc(Nc3ccc(N4CCC(C(F)(F)F)CC4)cc3)ccc21]

The title compound 364 (89.9 mg) was prepared in a total yield of 71.9% as a light yellow solid from 4-(4-(trifluoromethyl)piperidin-1-yl)aniline (105 mg, 0.43 mmol), 5-bromo-1-methyl-1H-indazole (70 mg, 0.33 mmol), Pd₂(dba)₃ (3.0 mg, 3.3 umol), X-Phos (7.8 mg, 16.5 umol), and Cs₂CO₃ (163 mg, 0.50 mmol) according to the procedure for compound 1. ¹H NMR (400 MHz, Methanol-d₄) δ 7.80 (s, 1H), 7.47-6.83 (m, 7H), 4.02 (s, 3H), 3.76-3.41 (m, 2H), 3.22-2.34 (m, 2H), 2.33-2.20 (m, 2H), 2.02-1.93 (m, 2H), 1.73 (qd, J=12.7, 3.9 Hz, 2H). Mass (m/z): 375.3 [M+H]⁺.

### Compound 365

### N-ethyl-1-methyl-5-((4-(4-methylpiperidin-1-yl)phenyl)amino)-1H-indole-3-carboxamide

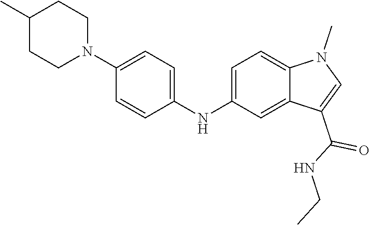 [SMILES: CCNC(=O)c1cn(C)c2ccc(Nc3ccc(N4CCC(C)CC4)cc3)cc12]

The title compound 365 (12.0 mg) was prepared in a total yield of 9.2% as a gray solid from 4-(4-methylpiperidin-1-yl)aniline (81.7 mg, 0.43 mmol), 5-bromo-N-ethyl-1-methyl-1H-indole-3-carboxamide (93.3 mg, 0.33 mmol), Pd₂(dba)₃ (3.0 mg, 3.3 umol), X-Phos (7.8 mg, 16.5 umol), and Cs₂CO₃ (163 mg, 0.50 mmol) according to the procedure for compound 1. ¹H NMR (400 MHz, Methanol-d₄) δ 7.71 (s, 1H), 7.55-6.6 (m, 7H), 4.00-3.75 (m, 4H), 3.39 (q, J=7.2 Hz, 2H), 3.33 (s, 3H), 1.88-1.34 (m, 5H), 1.22 (t, J=7.2 Hz, 3H), 1.02 (d, J=6.1 Hz, 3H). Mass (m/z): 391.3 [M+H]⁺.

### Compound 366

### N-(4-(4-(trifluoromethyl)piperidin-1-yl)phenyl)-1H-indazol-6-amine

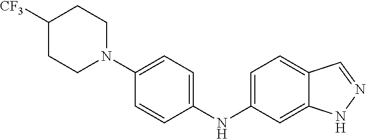 [SMILES: FC(F)(F)C1CCN(c2ccc(Nc3ccc4cn[nH]c4c3)cc2)CC1]

To a solution of tert-butyl 6-((4-(4-(trifluoromethyl)piperidin-1-yl) phenyl)amino)-1H-indazole-1-carboxylate (45 mg, 0.10 mmol) in EtOH (5 mL) was added a solution of HCl in 1,4-dioxane (5 mL). Then the solution was stirred for 30 mins at r.t. and concentrated. 10 ml water was added. The pH of the filtration was adjusted to 8-9 with sodium carbonate solution. Then the mixture was extracted by DCM (15 mL×3). The combined organic layers were washed with water (10 mL), dried over Na₂SO₄ and concentrated. The residue was purified by perp-TLC (MeOH/DCM=1/10) to afford the desired product as a light gray solid. (23.0 mg, 63.8%). ¹H NMR (400 MHz, Methanol-d₄) δ 8.56 (s, 1H), 8.04-6.31 (m, 7H), 4.01-3.51 (m, 2H), 3.11-2.57 (m, 2H), 2.41-2.27 (m, 1H), 2.13-1.65 (m, 4H). ¹H NMR (400 MHz, Methanol-d₄) δ 8.56 (s, 1H), 7.87 (d, J=9.0 Hz, 1H), 7.78-7.70 (m, 2H), 7.50-7.41 (m, 2H), 7.23-7.14 (m, 1H), 3.85-3.77 (m, 4H), 2.91-2.78 (m, 1H), 2.36-2.20 (m, 4H). Mass (m/z): 361.2 [M+H]⁺.

### Compound 367

### 3-methyl-1-(2-(2-oxopyrrolidin-1-yl)ethyl)-5-((4-(4-(trifluoromethyl) piperidin-1-yl)phenyl)amino)-1,3-dihydro-2H-benzo[d]imidazol-2-one

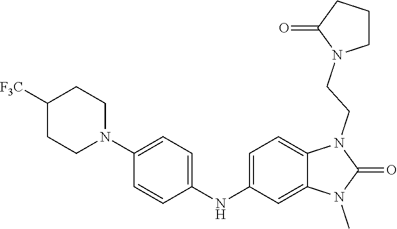 [SMILES: Cn1c(=O)n(CCN2CCCC2=O)c2ccc(Nc3ccc(N4CCC(C(F)(F)F)CC4)cc3)cc21]

The title compound 367 (22.1 mg) was prepared in a total yield of 87.9% as a yellow solid from 4-(4-(trifluoromethyl)piperidin-1-yl)aniline (15.9 mg, 0.065 mmol), 5-bromo-3-methyl-1-(2-(2-oxopyrrolidin-1-yl) ethyl)-1,3-dihydro-2H-benzo[d]imidazol-2-one (16.9 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, Methanol-d₄) δ 7.35-6.54 (m, 7H), 4.07-3.96 (m, 2H), 3.59 (t, J=6.8 Hz, 2H), 3.43 (t, J=6.8 Hz, 2H), 3.33 (s, 3H), 2.81-2.65 (m, 2H), 2.21 (m, 1H), 2.19 (t, J=8.1 Hz, 2H), 2.05-1.86 (m, 4H), 1.82-1.62 (m, 2H). Mass (m/z): 502.3 [M+H]⁺

### Compound 368

### 1,3-dimethyl-5-((4-(2-methylpiperidin-1-yl)phenyl)amino)-1,3-dihydro-2H-benzo[d]imidazol-2-one

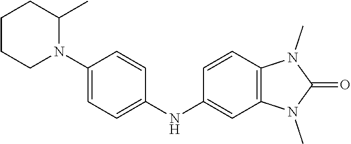 [SMILES: CC1CCCCN1c1ccc(Nc2ccc3c(c2)n(C)c(=O)n3C)cc1]

The title compound 368 (31.9 mg) was prepared in a total yield of 43.8% as a green solid from 4-(2-methylpiperidin-1-yl)aniline (48 mg, 0.250 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, DMSO-d₆) δ 7.78 (s, 1H), 7.14-6.63 (m, 7H), 3.50 (s, 1H), 3.27 (s, 6H), 3.00 (s, 2H), 1.89-1.33 (m, 6H), 0.88 (s, 3H). Mass (m/z): 321.3 [M+H]⁺.

### Compound 369

### 5-((4-((2-(2-methoxyethoxy)ethyl)(methyl)amino)phenyl)amino)-1,3-dimethyl-1,3-dihydro-2H-benzo[d]imidazol-2-one

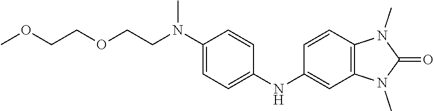 [SMILES: COCCOCCN(C)c1ccc(Nc2ccc3c(c2)n(C)c(=O)n3C)cc1]

The title compound 369 (41.8 mg) was prepared in a total yield of 52.3% as a brown solid from N¹-(2-(2-methoxyethoxy)ethyl)-N¹-methylbenzene-1,4-diamine (56 mg, 0.250 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, DMSO-d₆) δ 7.45 (s, 1H), 6.94 (t, J=9.2 Hz, 3H), 6.72-6.56 (m, 4H), 3.57-3.49 (m, 4H), 3.45-3.38 (m, 4H), 3.24 (d, J=3.2 Hz, 6H), 2.86 (s, 3H). Mass (m/z): 385.3 [M+H]⁺.

### Compound 370

### 1-methyl-N⁵-(4-(4-(trifluoromethyl)piperidin-1-yl)phenyl)-1H-benzo[d]imidazole-2,5-diamine

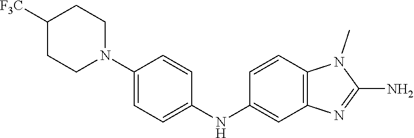 [SMILES: Cn1c(N)nc2cc(Nc3ccc(N4CCC(C(F)(F)F)CC4)cc3)ccc21]

The title compound 370 (2.0 mg) was prepared in a total yield of 1.5% as a light pink solid from 4-(4-(trifluoromethyl)piperidin-1-yl)aniline (105 mg, 0.43 mmol), 5-bromo-1-methyl-1H-benzo[d]imidazol-2-amine (75 mg, 0.33 mmol), Pd₂(dba)₃ (3.0 mg, 3.3 umol), X-Phos (7.8 mg, 16.5 umol), and t-BuONa (48 mg, 0.50 mmol) according to the procedure for compound 1. ¹H NMR (400 MHz, Methanol-d₄) δ 7.13-6.52 (m, 7H), 3.73-3.44 (m, 5H), 2.85-2.49 (m, 2H), 2.34-2.21 (m, 1H), 2.02-1.91 (m, 2H), 1.80-1.67 (m, 2H). Mass (m/z): 390.3 [M+H]⁺.

### Compound 371

### 1,3-dimethyl-5-((5-methyl-6-(4-(trifluoromethyl)piperidin-1-yl)pyridin-3-yl)amino)-1,3-dihydro-2H-benzo[d]imidazol-2-one

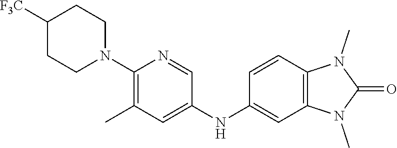 [SMILES: Cc1cc(Nc2ccc3c(c2)n(C)c(=O)n3C)cnc1N1CCC(C(F)(F)F)CC1]

The title compound 371 (33.4 mg) was prepared in a total yield of 31.9% as a light yellow powder from 5-methyl-6-(4-(trifluoromethyl) piperidin-1-yl)pyridin-3-amine (84 mg, 0.33 mmol), 5-bromo-1,3-dimethyl-1H-benzo[d]imidazol-2(3H)-one (60 mg, 0.25 mmol), Pd₂(dba)₃ (2.0 mg, 2.5 umol), X-Phos (6.0 mg, 12.5 umol), and t-BuONa (36 mg, 0.38 mmol) according to the procedure for compound 1. ¹H NMR (400 MHz, Methanol-d₄) δ 7.88 (d, J=2.8 Hz, 1H), 7.31 (dd, J=2.8, 0.9 Hz, 1H), 7.08-7.02 (m, 1H), 6.89-6.83 (m, 2H), 3.41 (s, 3H), 3.38 (s, 3H), 3.37-3.34 (m, 2H), 2.79 (td, J=12.4, 2.3 Hz, 2H), 2.37-2.29 (m, 1H), 2.28 (s, 3H), 2.01-1.94 (m, 2H), 1.69-1.57 (m, 2H). Mass (m/z): 420.3 [M+H]⁺.

### Compound 372

### 5-((5-methoxy-6-(4-(trifluoromethyl)piperidin-1-yl)pyridin-3-yl)amino)-1,3-dimethyl-1,3-dihydro-2H-benzo[d]imidazol-2-one

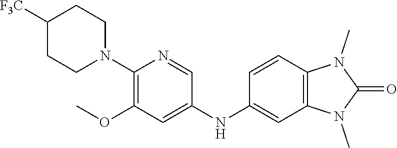 [SMILES: COc1cc(Nc2ccc3c(c2)n(C)c(=O)n3C)cnc1N1CCC(C(F)(F)F)CC1]

The title compound 372 (21.7 mg) was prepared in a total yield of 19.9% as a light gray powder from 5-methoxy-6-(4-(trifluoromethyl) piperidin-1-yl)pyridin-3-amine (84 mg, 0.33 mmol), 5-bromo-1,3-dimethyl-2,3-dihydro-1H-1,3-benzodiazol-2-one (60 mg, 0.25 mmol), Pd₂(dba)₃ (2.0 mg, 2.5 umol), X-Phos (6.0 mg, 12.5 umol), and t-BuONa (36 mg, 0.38 mmol) according to the procedure for compound 1. ¹H NMR (400 MHz, Methanol-d₄) δ 7.61 (d, J=2.3 Hz, 1H), 7.06 (d, J=5.7 Hz, 1H), 7.05 (s, 1H), 6.91-6.86 (m, 2H), 3.85 (s, 3H), 3.82-3.73 (m, 2H), 3.41 (s, 3H), 3.39 (s, 3H), 2.72 (td, J=12.5, 2.4 Hz, 2H), 2.36-2.25 (m, 1H), 1.98-1.89 (m, 2H), 1.69-1.57 (m, 2H). Mass (m/z): 436.3 [M+H]⁺.

### Compound 373

### 1,3-dimethyl-5-((2-methyl-6-(4-(trifluoromethyl)piperidin-1-yl)pyridin-3-yl)amino)-1,3-dihydro-2H-benzo[d]imidazol-2-one

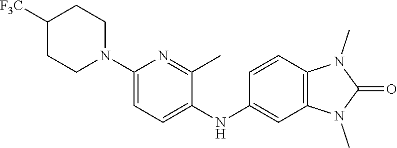 [SMILES: Cc1nc(N2CCC(C(F)(F)F)CC2)ccc1Nc1ccc2c(c1)n(C)c(=O)n2C]

The title compound 373 (54.4 mg) was prepared in a total yield of 51.8% as a light yellow powder from 2-methyl-6-(4-(trifluoromethyl) piperidin-1-yl)pyridin-3-amine (84 mg, 0.33 mmol), 5-bromo-1,3-dimethyl-2,3-dihydro-1H-1,3-benzodiazol-2-one (60 mg, 0.25 mmol), Pd₂(dba)₃ (2.3 mg, 2.5 umol), X-Phos (6.0 mg, 12.5 umol), and t-BuONa (36 mg, 0.38 mmol) according to the procedure for compound 1. ¹H NMR (400 MHz, Methanol-d₄) δ 7.39 (d, J=8.7 Hz, 1H), 6.94 (d, J=8.4 Hz, 1H), 6.68 (d, J=8.8 Hz, 1H), 6.52 (dd, J=8.4, 2.1 Hz, 1H), 6.47 (d, J=2.1 Hz, 1H), 4.35 (d, J=12.9 Hz, 2H), 3.38 (s, 3H), 2.81 (t, J=12.7 Hz, 2H), 2.47-2.35 (m, 1H), 2.33 (s, 3H), 2.00-1.89 (m, 1H), 1.69-1.57 (m, 2H). Mass (m/z): 420.3 [M+H]⁺.

### Compound 374

### 2-((1,3-dimethyl-2-oxo-2,3-dihydro-1H-benzo[d]imidazol-5-yl)amino)-5-(4-(trifluoromethyl)piperidin-1-yl)benzonitrile

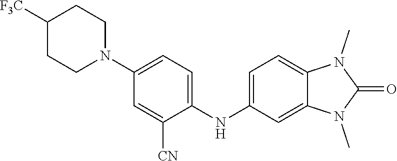 [SMILES: Cn1c(=O)n(C)c2cc(Nc3ccc(N4CCC(C(F)(F)F)CC4)cc3C#N)ccc21]

The title compound 374 (45.4 mg) was prepared in a yield of 56.93% as a white powder from 2-amino-5-(4-(trifluoromethyl)piperidin-1-yl) benzonitrile (50 mg, 0.19 mmol) and 5-bromo-1,3-dimethyl-1,3-dihydro-2H-benzo[d]imidazol-2-one (49 mg, 0.20 mmol) according to the procedure for compound 1. ¹H NMR (Methanol-d₄) δ 7.20 (dd, J=9.1, 2.9 Hz, 1H), 7.16-7.13 (m, 1H), 7.10 (d, J=9.1 Hz, 1H), 7.06 (d, J=8.9 Hz, 1H), 6.90-6.86 (m, 2H), 3.63 (d, J=11.9 Hz, 2H), 3.41 (s, 3H), 3.37 (s, 3H), 2.68 (td, J=12.3, 2.5 Hz, 2H), 2.29 (ddp, J=12.4, 7.9, 4.0 Hz, 1H), 1.98 (d, J=12.9 Hz, 2H), 1.78-1.62 (m, 2H). Mass (m/z): 430.5 [M+H]⁺.

### Compound 375

### 5-((2-methoxy-4-(4-(trifluoromethyl)piperidin-1-yl)phenyl)amino)-1,3-dimethyl-1,3-dihydro-2H-benzo[d]imidazol-2-one

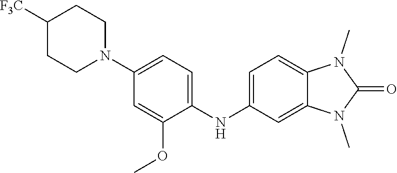 [SMILES: COc1cc(N2CCC(C(F)(F)F)CC2)ccc1Nc1ccc2c(c1)n(C)c(=O)n2C]

The title compound 375 (36.7 mg) was prepared in a yield of 46.34% as a white powder from 2-methoxy-4-(4-(trifluoromethyl)piperidin-1-yl)aniline (50 mg, 0.18 mmol) and 5-bromo-1,3-dimethyl-1,3-dihydro-2H-benzo[d]imidazol-2-one (48 mg, 0.20 mmol) according to the procedure for compound 1. ¹H NMR (Methanol-d₄) δ: 7.30-6.28 (m, 6H), 3.87 (s, 5H), 3.41-3.36 (m, 3H), 2.21 (s, 2H), 2.11-1.85 (m, 4H), 1.66 (d, J=48.8 Hz, 4H). Mass (m/z): 435.2 [M+H]⁺.

### Compound 376

### 1,3-dimethyl-5-((2-(4-methylpiperidin-1-yl)pyrimidin-5-yl)amino)-1,3-dihydro-2H-benzo[d]imidazol-2-one

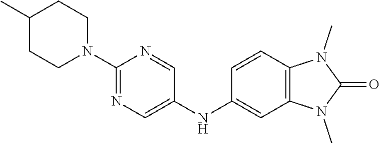 [SMILES: CC1CCN(c2ncc(Nc3ccc4c(c3)n(C)c(=O)n4C)cn2)CC1]

The title compound 376 (10.1 mg) was prepared in a total yield of 13.8% as a white solid from 2-(4-methylpiperidin-1-yl)pyrimidin-5-amine (48 mg, 0.250 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, DMSO-d₆) δ 8.23 (s, 2H), 7.49 (s, 1H), 6.94 (d, J=8.4 Hz, 1H), 6.61 (d, J=2.0 Hz, 1H), 6.54 (dd, J=8.4, 2.0 Hz, 1H), 4.55 (dt, J=12.4, 3.2 Hz, 2H), 3.25 (d, J=7.2 Hz, 6H), 2.82 (td, J=12.8, 2.4 Hz, 2H), 1.70-1.60 (m, 3H), 1.12-1.00 (m, 2H), 0.93 (d, J=6.4 Hz, 3H). Mass (m/z): 353.3 [M+H]⁺.

### Compound 377

### 5-((2-methoxy-6-(4-(trifluoromethyl)piperidin-1-yl)pyridin-3-yl)amino)-1,3-dimethyl-1,3-dihydro-2H-benzo[d]imidazol-2-one

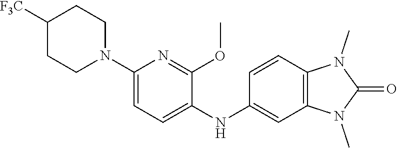 [SMILES: COc1nc(N2CCC(C(F)(F)F)CC2)ccc1Nc1ccc2c(c1)n(C)c(=O)n2C]

The title compound 377 (40.6 mg) was prepared in a total yield of 37.3% as a purple powder from 2-methoxy-6-(4-(trifluoromethyl) piperidin-1-yl) pyridin-3-amine (79 mg, 0.29 mmol), 5-bromo-1,3-dimethyl-2,3-dihydro-1H-1,3-benzodiazol-2-one (60 mg, 0.25 mmol), Pd₂(dba)₃ (2.3 mg, 2.5 umol), X-Phos (6.0 mg, 12.5 umol), and t-BuONa (36 mg, 0.38 mmol) according to the procedure for compound 1. ¹H NMR (400 MHz, Methanol-d₄) δ 7.38 (d, J=8.3 Hz, 1H), 6.96 (d, J=8.4 Hz, 1H), 6.71 (d, J=8.4 Hz, 1H), 6.67 (d, J=2.1 Hz, 1H), 6.31 (d, J=8.3 Hz, 1H), 4.38-4.32 (m, 2H), 3.93 (s, 3H), 3.38 (s, 3H), 3.34 (s, 3H), 2.79 (t, J=12.8 Hz, 2H), 2.44-2.35 (m, 1H), 1.98-1.92 (m, 2H), 1.69-1.57 (m, 2H). Mass (m/z): 436.3 [M+H]⁺.

### Compound 378

### 5-((5-fluoro-6-(4-(trifluoromethyl)piperidin-1-yl)pyridin-3-yl)amino)-1,3-dimethyl-1,3-dihydro-2H-benzo[d]imidazol-2-one

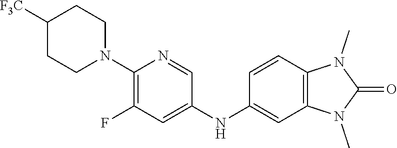 [SMILES: Cn1c(=O)n(C)c2cc(Nc3cnc(N4CCC(C(F)(F)F)CC4)c(F)c3)ccc21]

The title compound 378 (40.6 mg) was prepared in a total yield of 42.0% as a brown powder from 5-fluoro-6-(4-(trifluoromethyl)piperidin-1-yl) pyridin-3-amine (75.6 mg, 0.29 mmol), 5-bromo-1,3-dimethyl-2,3-dihydro-1H-1,3-benzodiazol-2-one (60 mg, 0.25 mmol), Pd₂(dba)₃ (2.3 mg, 2.5 umol), X-Phos (6.0 mg, 12.5 umol), and t-BuONa (36 mg, 0.38 mmol) according to the procedure for compound 1. ¹H NMR (400 MHz, Methanol-d₄) δ 7.80 (d, J=2.1 Hz, 1H), 7.18 (dd, J=13.9, 2.4 Hz, 1H), 7.06-7.01 (m, 1H), 6.89-6.83 (m, 2H), 3.85-3.77 (m, 2H), 3.40 (s, 3H), 3.38 (s, 3H), 2.83 (td, J=12.6, 2.4 Hz, 2H), 2.39-2.26 (m, 1H), 1.99-1.89 (m, 2H), 1.78-1.65 (m, 2H). Mass (m/z): 424.3 [M+H]⁺.

### Compound 379

### 1,3-dimethyl-5-((4-methyl-6-(4-(trifluoromethyl)piperidin-1-yl)pyridin-3-yl)amino)-1,3-dihydro-2H-benzo[d]imidazol-2-one

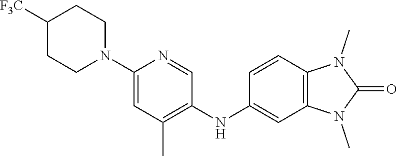 [SMILES: Cc1cc(N2CCC(C(F)(F)F)CC2)ncc1Nc1ccc2c(c1)n(C)c(=O)n2C]

The title compound 379 (49.7 mg) was prepared in a total yield of 47.3% as a peach puff powder from 4-methyl-6-(4-(trifluoromethyl) piperidin-1-yl)pyridin-3-amine (75.0 mg, 0.29 mmol), 5-bromo-1,3-dimethyl-2,3-dihydro-1H-1,3-benzodiazol-2-one (60 mg, 0.25 mmol), Pd₂(dba)₃ (2.3 mg, 2.5 umol), X-Phos (6.0 mg, 12.5 umol), and t-BuONa (36 mg, 0.38 mmol) according to the procedure for compound 1. ¹H NMR (400 MHz, Methanol-d₄) δ 7.92 (s, 1H), 6.95 (d, J=8.4 Hz, 1H), 6.82 (s, 1H), 6.51 (dd, J=8.4, 2.1 Hz, 1H), 6.48 (d, J=2.1 Hz, 1H), 4.34-4.24 (m, 2H), 3.38 (s, 3H), 3.32 (s, 3H), 2.90-2.81 (m, 2H), 2.48-2.36 (m, 1H), 2.21 (s, 3H), 2.00-1.92 (m, 2H), 1.68-1.55 (m, 2H). Mass (m/z): 420.3 [M+H]⁺.

### Compound 380

### 5-((4-methoxy-6-(4-(trifluoromethyl)piperidin-1-yl)pyridin-3-yl)amino)-1,3-dimethyl-1,3-dihydro-2H-benzo[d]imidazol-2-one

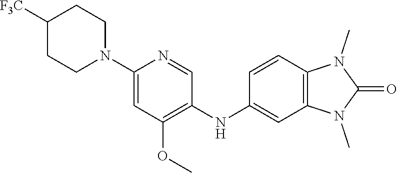 [SMILES: COc1cc(N2CCC(C(F)(F)F)CC2)ncc1Nc1ccc2c(c1)n(C)c(=O)n2C]

The title compound 380 (27.0 mg) was prepared in a total yield of 21.6% as a light yellow powder from 4-methoxy-6-(4-(trifluoromethyl) piperidin-1-yl)pyridin-3-amine (69.0 mg, 0.29 mmol), 5-bromo-1,3-dimethyl-2,3-dihydro-1H-1,3-benzodiazol-2-one (60 mg, 0.25 mmol), Pd₂(dba)₃ (2.3 mg, 2.5 umol), X-Phos (6.0 mg, 12.5 umol), and t-BuONa (36 mg, 0.38 mmol) according to the procedure for compound 1. ¹H NMR (400 MHz, Methanol-d₄) δ 7.85 (s, 1H), 6.98 (d, J=8.3 Hz, 1H), 6.71 (dd, J=8.3, 2.1 Hz, 1H), 6.68 (d, J=2.1 Hz, 1H), 6.52 (d, J=6.9 Hz, 1H), 4.32-4.24 (m, 2H), 3.92 (s, 3H), 3.39 (s, 3H), 3.35 (s, 3H), 2.92-2.85 (m, 2H), 2.48-2.40 (m, 1H), 2.02-1.99 (m, 2H), 1.67-1.61 (m, 2H). Mass (m/z): 436.3 [M+H]⁺.

### Compound 381

### 5-((3-methoxy-4-(4-(trifluoromethyl)piperidin-1-yl)phenyl)amino)-1,3-dimethyl-1,3-dihydro-2H-benzo[d]imidazol-2-one

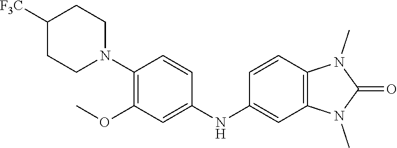 [SMILES: COc1cc(Nc2ccc3c(c2)n(C)c(=O)n3C)ccc1N1CCC(C(F)(F)F)CC1]

The title compound 381 (37.8 mg) was prepared in a total yield of 41.8% as a yellow solid from 3-methoxy-4-(4-(trifluoromethyl)piperidin-1-yl)aniline (65 mg, 0.250 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (300 MHz, DMSO-d₆) δ 7.10-6.46 (m, 6H), 3.82 (s, 3H), 3.48 (s, 3H), 3.22 (s, 6H), 2.72 (s, 1H), 2.12-1.81 (m, 5H). Mass (m/z): 435.3 [M+H]⁺.

### Compound 382

### 1,3-dimethyl-5-(phenylamino)-1,3-dihydro-2H-benzo[d]imidazol-2-one

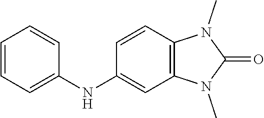 [SMILES: Cn1c(=O)n(C)c2cc(Nc3ccccc3)ccc21]

The title compound 382 (41.7 mg) was prepared in a total yield of 79.1% as a yellow solid from aniline (22 mg, 0.250 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, DMSO-d₆) δ 7.96 (s, 1H), 7.20-7.14 (m, 2H), 7.03 (d, J=8.4 Hz, 1H), 7.00-6.95 (m, 2H), 6.89 (d, J=2.0 Hz, 1H), 6.81 (dd, J=8.4, 2.0 Hz, 1H), 6.72 (tt, J=7.2, 1.1 Hz, 1H), 3.29 (d, J=7.6 Hz, 6H). Mass (m/z): 254.3 [M+H]⁺.

### Compound 383

### 1-methyl-5-((4-(4-(trifluoromethyl)piperidin-1-yl)phenyl)amino)-1H-indole-3-carboxamide

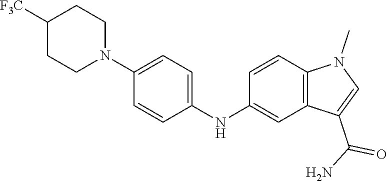 [SMILES: Cn1cc(C(N)=O)c2cc(Nc3ccc(N4CCC(C(F)(F)F)CC4)cc3)ccc21]

The title compound 383 (3.7 mg) was prepared in a yield of 4.5% as a white powder from 4-(4-(trifluoromethyl)piperidin-1-yl)aniline (50 mg, 0.20 mmol) and 5-bromo-1-methyl-1H-indole-3-carboxamide (48 mg, 0.20 mmol) according to the procedure for compound 1. ¹H NMR (DMSO-d₆) δ 7.93 (s, 2H), 7.34 (s, 2H), 6.81 (d, J=119.4 Hz, 4H), 3.92 (s, 4H), 3.16 (s, 1H), 2.72-2.63 (m, 1H), 2.32 (p, J=1.9 Hz, 1H), 1.99 (q, J=6.9, 6.3 Hz, 3H), 1.42 (s, 1H), 0.90-0.79 (m, 1H). Mass (m/z): 417.4 [M+H]⁺.

### Compound 384

### 5-((4-(4-methoxy-4-(trifluoromethyl)piperidin-1-yl)phenyl)amino)-1,3-dimethyl-1,3-dihydro-2H-benzo[d]imidazol-2-one

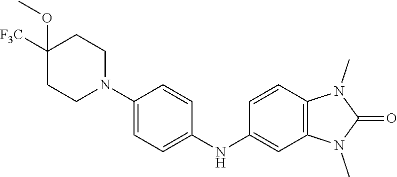 [SMILES: COC1(C(F)(F)F)CCN(c2ccc(Nc3ccc4c(c3)n(C)c(=O)n4C)cc2)CC1]

The title compound 384 (30.2 mg) was prepared in a total yield of 33.4% as a yellow solid from 4-(4-methoxy-4-(trifluoromethyl)piperidin-1-yl) aniline (68 mg, 0.250 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, Pyridine-d₅) δ 8.90 (s, 2H), 8.60 (s, 3H), 8.46 (s, 1H), 8.37 (d, J=8.4 Hz, 1H), 4.95 (s, 2H), 4.86 (q, J=1.2 Hz, 3H), 4.78 (s, 3H), 4.72 (s, 3H), 4.40 (s, 2H), 3.54-3.37 (m, 4H). Mass (m/z): 435.3 [M+H]⁺.

### Compound 385

### 5-((4-(4-(fluoromethyl)piperidin-1-yl)phenyl)amino)-1,3-dimethyl-1,3-dihydro-2H-benzo[d]imidazol-2-one

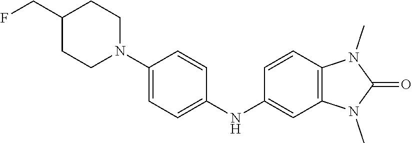 [SMILES: Cn1c(=O)n(C)c2cc(Nc3ccc(N4CCC(CF)CC4)cc3)ccc21]

The title compound 385 (19.4 mg) was prepared in a total yield of 25.3% as a blue solid from 4-(4-(fluoromethyl)piperidin-1-yl)aniline (52 mg, 0.250 mmol), 5-bromo-1,3-dimethyl-1,3-dihydro-2H-benzo[d]imidazol-2-one (50 mg, 0.208 mmol), Pd₂(dba)₃ (2 mg, 0.002 mmol), X-phos (6 mg, 0.01 mmol), Cs₂CO₃ (102 mg, 0.312 mmol) and 1,4-dioxane (5 mL) according to the procedure for compound 1. ¹H NMR (400 MHz, DMSO-d₆) δ 7.83 (s, 1H), 7.12 (d, J=8.4 Hz, 1H), 6.99 (d, J=8.4 Hz, 2H), 6.89 (d, J=8.4 Hz, 2H), 6.74 (s, 1H), 6.63 (d, J=8.8 Hz, 1H), 4.40 (d, J=6.0 Hz, 1H), 4.28 (d, J=5.6 Hz, 1H), 3.57 (d, J=12.0 Hz, 2H), 3.24 (d, J=3.2 Hz, 6H), 2.60 (t, J=12.0 Hz, 2H), 1.74 (d, J=13.2 Hz, 3H), 1.36 (d, J=12.4 Hz, 2H). Mass (m/z): 369.3 [M+H]⁺.

### Compound 386

### 6-((4-(4-methoxy-4-(trifluoromethyl)piperidin-1-yl)phenyl)amino)-3-methylbenzo[d]oxazol-2(3H)-one

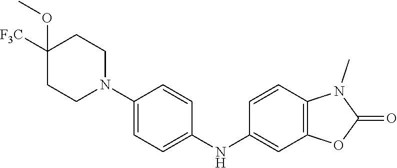 [SMILES: COC1(C(F)(F)F)CCN(c2ccc(Nc3ccc4c(c3)oc(=O)n4C)cc2)CC1]

The title compound 386 (23.6 mg) was prepared in a total yield of 25.6% as a yellow solid from 4-(4-methoxy-4-(trifluoromethyl)piperidin-1-yl) aniline (72 mg, 0.263 mmol), 6-bromo-3-methylbenzo[d]oxazol-2(3H)-one (50 mg, 0.219 mmol), Pd₂(dba)₃ (2 mg, 0.002 mmol), X-phos (6 mg, 0.01 mmol), Cs₂CO₃ (129 mg, 0.394 mmol) and 1,4-dioxane (5 mL) according to the procedure for compound 1. ¹H NMR (400 MHz, DMSO-d₆) δ 7.83 (s, 1H), 7.06 (d, J=8.4 Hz, 1H), 6.99-6.88 (m, 5H), 6.79 (dd, J=8.4, 2.0 Hz, 1H), 3.46 (d, J=12.4 Hz, 2H), 3.39 (d, J=1.2 Hz, 3H), 3.29 (s, 3H), 2.75 (td, J=12.4, 2.4 Hz, 2H), 2.03-1.95 (m, 2H), 1.83 (td, J=13.2, 4.4 Hz, 2H). Mass (m/z): 422.3 [M+H]⁺.

### Compound 387

### 6-((4-(4-(fluoromethyl)piperidin-1-yl)phenyl)amino)-3-methylbenzo[d]oxazol-2(3H)-one

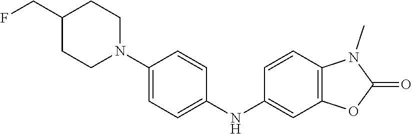 [SMILES: Cn1c(=O)oc2cc(Nc3ccc(N4CCC(CF)CC4)cc3)ccc21]

The title compound 387 (29.7 mg) was prepared in a total yield of 38.2% as a white solid from 4-(4-(fluoromethyl)piperidin-1-yl)aniline (55 mg, 0.263 mmol), 6-bromo-3-methylbenzo[d]oxazol-2(3H)-one (50 mg, 0.219 mmol), Pd₂(dba)₃ (2 mg, 0.002 mmol), X-phos (6 mg, 0.01 mmol), Cs₂CO₃ (129 mg, 0.394 mmol) and 1,4-dioxane (5 mL) according to the procedure for compound 1. ¹H NMR (400 MHz, DMSO-d₆) δ 7.83 (s, 1H), 7.12 (d, J=8.4 Hz, 1H), 6.99 (d, J=8.4 Hz, 2H), 6.89 (d, J=8.4 Hz, 2H), 6.74 (s, 1H), 6.63 (d, J=8.8 Hz, 1H), 4.40 (d, J=6.0 Hz, 1H), 4.28 (d, J=5.6 Hz, 1H), 3.57 (d, J=11.6 Hz, 2H), 3.27 (s, 3H), 2.60 (t, J=12.0 Hz, 2H), 1.74 (d, J=13.2 Hz, 3H), 1.36 (d, J=12.4 Hz, 2H). Mass (m/z): 356.3 [M+H]⁺.

### Compound 388

### 5-((5-fluoro-6-(4-(trifluoromethyl)piperidin-1-yl)pyridin-3-yl)amino)-3-methylbenzo[d]oxazol-2(3H)-one

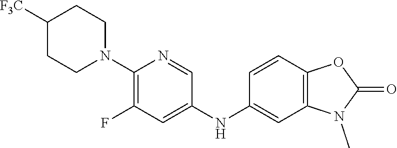 [SMILES: Cn1c(=O)oc2ccc(Nc3cnc(N4CCC(C(F)(F)F)CC4)c(F)c3)cc21]

The title compound 388 (5.3 mg) was prepared in a total yield of 5.2% as a light yellow powder from 5-fluoro-6-(4-(trifluoromethyl)piperidin-1-yl)pyridin-3-amine (87.0 mg, 0.33 mmol), 5-bromo-3-methylbenzo[d]oxazol-2(3H)-one (57 mg, 0.25 mmol), Pd₂(dba)₃ (2.3 mg, 2.5 umol), X-Phos (6.0 mg, 12.5 umol), and Cs₂CO₃ (122 mg, 0.38 mmol) according to the procedure for compound 1. ¹H NMR (400 MHz, DMSO-d₆) δ 8.19 (s, 1H), 7.89 (dd, J=2.4, 1.0 Hz, 1H), 7.30 (dd, J=14.2, 2.4 Hz, 1H), 7.19 (d, J=8.6 Hz, 1H), 6.89 (d, J=2.3 Hz, 1H), 6.71 (dd, J=8.6, 2.3 Hz, 1H), 3.81-3.76 (m, 2H), 3.31 (s, 3H), 2.86-2.75 (m, 2H), 2.59-2.52 (m, 1H), 1.92-1.84 (m, 2H), 1.63-1.51 (m, 2H). Mass (m/z): 411.2 [M+H]⁺.

### Compound 389

### 3-methyl-5-((4-(piperidin-1-yl)phenyl)amino)benzo[d]oxazol-2(3H)-one

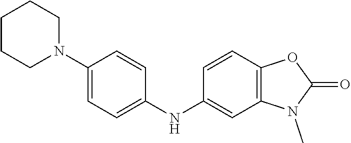 [SMILES: Cn1c(=O)oc2ccc(Nc3ccc(N4CCCCC4)cc3)cc21]

The title compound 389 (56.0 mg) was prepared in a total yield of 79.1% as a white solid from 4-(piperidin-1-yl)aniline (46 mg, 0.263 mmol), 5-bromo-3-methylbenzo[d]oxazol-2(3H)-one (50 mg, 0.219 mmol), Pd₂(dba)₃ (2 mg, 0.002 mmol), X-phos (6 mg, 0.01 mmol), Cs₂CO₃ (129 mg, 0.394 mmol) and 1,4-dioxane (5 mL) according to the procedure for compound 1. ¹H NMR (400 MHz, DMSO-d₆) δ 7.77 (s, 1H), 7.04 (d, J=8.4 Hz, 1H), 6.96-6.88 (m, 2H), 6.80 (d, J=8.0 Hz, 2H), 6.67 (s, 1H), 6.55 (d, J=8.8 Hz, 1H), 3.19 (s, 3H), 2.94 (s, 4H), 1.55 (s, 4H), 1.43 (s, 2H). Mass (m/z): 324.3 [M+H]⁺.

### Compound 390

### 5-((4-(4-methoxy-4-(trifluoromethyl)piperidin-1-yl)phenyl)amino)-3-methylbenzo[d]oxazol-2(3H)-one

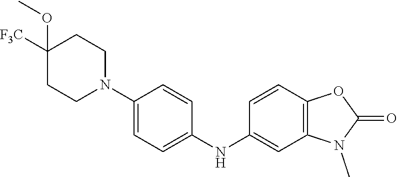 [SMILES: COC1(C(F)(F)F)CCN(c2ccc(Nc3ccc4oc(=O)n(C)c4c3)cc2)CC1]

The title compound 390 (52.0 mg) was prepared in a total yield of 56.3% as a white solid from 4-(4-methoxy-4-(trifluoromethyl)piperidin-1-yl)aniline (72 mg, 0.263 mmol), 5-bromo-3-methylbenzo[d]oxazol-2(3H)-one (50 mg, 0.219 mmol), Pd₂(dba)₃ (2 mg, 0.002 mmol), X-phos (6 mg, 0.01 mmol), Cs₂CO₃ (129 mg, 0.394 mmol) and 1,4-dioxane (5 mL) according to the procedure for compound 1. ¹H NMR (400 MHz, DMSO-d₆) δ 7.87 (s, 1H), 7.13 (d, J=8.4 Hz, 1H), 7.04-6.89 (m, 4H), 6.76 (s, 1H), 6.64 (d, J=8.8 Hz, 1H), 3.47 (d, J=12.0 Hz, 2H), 3.40 (d, J=1.2 Hz, 3H), 3.27 (s, 3H), 2.78 (d, J=10.4 Hz, 2H), 2.01 (d, J=13.6 Hz, 2H), 1.86 (d, J=13.6 Hz, 2H). Mass (m/z): 422.3 [M+H]⁺.

### Compound 391

### 5-((4-(4-(fluoromethyl)piperidin-1-yl)phenyl)amino)-3-methylbenzo[d]oxazol-2(3H)-one

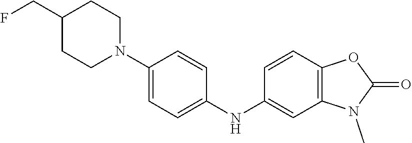 [SMILES: Cn1c(=O)oc2ccc(Nc3ccc(N4CCC(CF)CC4)cc3)cc21]

The title compound 391 (25.9 mg) was prepared in a total yield of 33.3% as a white solid from 4-(4-(fluoromethyl)piperidin-1-yl)aniline (55 mg, 0.263 mmol), 5-bromo-3-methylbenzo[d]oxazol-2(3H)-one (50 mg, 0.219 mmol), Pd₂(dba)₃ (2 mg, 0.002 mmol), X-phos (6 mg, 0.01 mmol), Cs₂CO₃ (129 mg, 0.394 mmol) and 1,4-dioxane (5 mL) according to the procedure for compound 1. ¹H NMR (400 MHz, DMSO-d₆) δ 7.83 (s, 1H), 7.12 (d, J=8.4 Hz, 1H), 6.99 (d, J=8.4 Hz, 2H), 6.89 (d, J=8.4 Hz, 2H), 6.74 (s, 1H), 6.63 (d, J=8.8 Hz, 1H), 4.40 (d, J=6.0 Hz, 1H), 4.28 (d, J=5.6 Hz, 1H), 3.57 (d, J=11.6 Hz, 2H), 3.27 (s, 3H), 2.60 (t, J=12.0 Hz, 2H), 1.74 (d, J=13.2 Hz, 3H), 1.36 (d, J=12.4 Hz, 2H). Mass (m/z): 356.3 [M+H]⁺.

### Compound 392

### 3-ethyl-5-((4-(4-(trifluoromethyl)piperidin-1-yl)phenyl)amino) benzo[d]oxazol-2(3H)-one

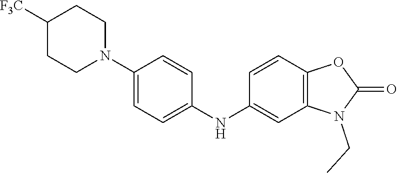 [SMILES: CCn1c(=O)oc2ccc(Nc3ccc(N4CCC(C(F)(F)F)CC4)cc3)cc21]

The title compound 392 (75.1 mg) was prepared in a total yield of 89.4% as a white solid from 4-(4-(trifluoromethyl)piperidin-1-yl)aniline (60 mg, 0.248 mmol), 5-bromo-3-ethylbenzo[d]oxazol-2(3H)-one (50 mg, 0.207 mmol), Pd₂(dba)₃ (2 mg, 0.002 mmol), X-phos (6 mg, 0.01 mmol), Cs₂CO₃ (122 mg, 0.372 mmol) and 1,4-dioxane (5 mL) according to the procedure for compound 1. ¹H NMR (400 MHz, DMSO-d₆) δ 7.92 (t, J=4.4 Hz, 1H), 7.13 (d, J=8.4 Hz, 1H), 7.02-6.97 (m, 2H), 6.93-6.88 (m, 2H), 6.81 (d, J=2.0 Hz, 1H), 6.65 (dd, J=8.4, 2.0 Hz, 1H), 3.78 (q, J=7.2 Hz, 2H), 3.62 (d, J=12.0 Hz, 2H), 2.63 (td, J=12.4, 2.4 Hz, 2H), 2.43 (ddq, J=12.4, 8.8, 4.0 Hz, 1H), 1.93-1.84 (m, 2H), 1.57 (qd, J=12.4, 4.0 Hz, 2H), 1.22 (t, J=7.2 Hz, 3H). Mass (m/z): 406.3 [M+H]⁺.

### Compound 393

### 3-(2-methoxyethyl)-5-((4-(4-(trifluoromethyl)piperidin-1-yl)phenyl) amino benzo[d]oxazol-2(3H)-one

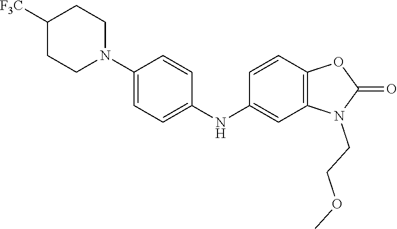 [SMILES: COCCn1c(=O)oc2ccc(Nc3ccc(N4CCC(C(F)(F)F)CC4)cc3)cc21]

The title compound 393 (58.0 mg) was prepared in a total yield of 72.5% as a white solid from 4-(4-(trifluoromethyl)piperidin-1-yl)aniline (54 mg, 0.221 mmol), 5-bromo-3-(2-methoxyethyl)benzo[d]oxazol-2(3H)-one (50 mg, 0.184 mmol), Pd₂(dba)₃ (2 mg, 0.002 mmol), X-phos (6 mg, 0.01 mmol), Cs₂CO₃ (91 mg, 0.276 mmol) and 1,4-dioxane (5 mL) according to the procedure for compound 1. ¹H NMR (400 MHz, DMSO-d₆) δ 7.92 (s, 1H), 7.13 (d, J=8.4 Hz, 1H), 7.02-6.96 (m, 2H), 6.93-6.84 (m, 3H), 6.64 (dd, J=8.8, 2.0 Hz, 1H), 3.91 (t, J=5.2 Hz, 2H), 3.65-3.56 (m, 4H), 3.25 (s, 3H), 2.63 (t, J=12.0 Hz, 2H), 2.47-2.37 (m, 1H), 1.88 (d, J=12.4 Hz, 2H), 1.58 (d, J=12.8 Hz, 2H). Mass (m/z): 436.3 [M+H]⁺.

### Compound 394

### 5-((4-cyclohexylphenyl)amino)-1,3-dimethyl-1,3-dihydro-2H-benzo[d]imidazol-2-one

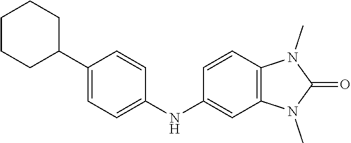 [SMILES: Cn1c(=O)n(C)c2cc(Nc3ccc(C4CCCCC4)cc3)ccc21]

The title compound 394 (49.7 mg) was prepared in a total yield of 71.6% as a yellow solid from 4-cyclohexylaniline (44 mg, 0.248 mmol), 5-bromo-1,3-dimethyl-1,3-dihydro-2H-benzo[d]imidazol-2-one (50 mg, 0.207 mmol), Pd₂(dba)₃ (2 mg, 0.002 mmol), X-phos (6 mg, 0.01 mmol), Cs₂CO₃ (102 mg, 0.310 mmol) and 1,4-dioxane (5 mL) according to the procedure for compound 1. ¹H NMR (400 MHz, DMSO-d₆) δ 7.86 (s, 1H), 7.05-6.99 (m, 3H), 6.95-6.89 (m, 2H), 6.85 (d, J=2.1 Hz, 1H), 6.78 (dd, J=8.3, 2.1 Hz, 1H), 3.28 (d, J=7.9 Hz, 6H), 2.42-2.42 (m, 1H), 1.82-1.66 (m, 5H), 1.42-1.28 (m, 4H), 1.22 (s, 1H). Mass (m/z): 336.3 [M+H]⁺.

### Compound 395

### 4-methoxy-1-methyl-N-(4-(4-(trifluoromethyl)piperidin-1-yl)phenyl)-1H-indol-6-amine

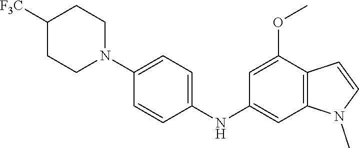 [SMILES: COc1cc(Nc2ccc(N3CCC(C(F)(F)F)CC3)cc2)cc2c1ccn2C]

The title compound 395 (17.1 mg) was prepared in a total yield of 84.8% as a yellow solid from 4-(4-(trifluoromethyl)piperidin-1-yl)aniline (15.9 mg, 0.065 mmol), 6-bromo-4-methoxy-1-methyl-1H-indole (12.0 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, DMSO-d₆) δ 7.10-6.78 (m, 5H), 6.58 (s, 1H), 6.26 (d, J=2.8 Hz, 2H), 3.80 (s, 3H), 3.60 (s, 3H), 3.58-3.51 (m, 2H), 2.69-2.52 (m, 2H), 2.38 (m, 1H), 1.94-1.79 (m, 2H), 1.66-1.49 (m, 2H). Mass (m/z): 404.2 [M+H]⁺.

### Compound 396

### N2-(cyclopropylmethyl)-1-methyl-N6-(4-(4-(trifluoromethyl)piperidin-1-yl) phenyl)-1H-indole-2,6-diamine

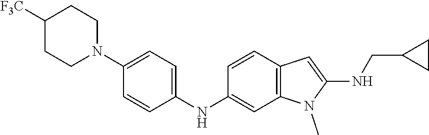 [SMILES: Cn1c(NCC2CC2)cc2ccc(Nc3ccc(N4CCC(C(F)(F)F)CC4)cc3)cc21]

The title compound 396 (17.1 mg) was prepared in a total yield of 84.8% as a yellow solid from 4-(4-(trifluoromethyl)piperidin-1-yl)aniline (15.9 mg, 0.065 mmol), 6-bromo-N-(cyclopropylmethyl)-1-methyl-1H-indol-2-amine (13.9 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, DMSO-d₆) δ 7.43-6.58 (m, 7H), 3.75-3.45 (m, 5H), 3.40-3.22 (m, 2H), 2.80-2.54 (m, 2H), 2.41 (m, 1H), 1.98-1.82 (m, 2H), 1.69-1.47 (m, 2H), 1.29-1.14 (m, 1H), 0.57-0.46 (m, 2H), 0.43-0.24 (m, 2H). Mass (m/z): 443.2 [M+H]⁺

### Compound 397

### N2,N2-bis(cyclopropylmethyl)-1-methyl-N6-(4-(4-(trifluoromethyl) piperidin-1-yl)phenyl)-1H-indole-2,6-diamine

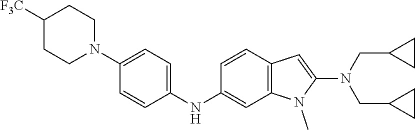 [SMILES: Cn1c(N(CC2CC2)CC2CC2)cc2ccc(Nc3ccc(N4CCC(C(F)(F)F)CC4)cc3)cc21]

The title compound 397 (20.3 mg) was prepared in a total yield of 81.8% as a yellow solid from 4-(4-(trifluoromethyl)piperidin-1-yl)aniline (15.9 mg, 0.065 mmol), 6-bromo-N,N-bis(cyclopropylmethyl)-1-methyl-1H-indol-2-amine (16.7 mg, 0.05 mmol), Pd₂(dba)₃ (0.92 mg, 0.001 mmol), X-Phos (2.38 mg, 0.005 mmol) and Cs₂CO₃ (24.4 mg, 0.075 mmol) according to the procedure for compound 1. ¹H NMR (400 MHz, DMSO-d₆) δ 7.40-6.49 (m, 8H), 3.70-3.42 (m, 5H), 3.16 (d, J=6.8 Hz, 4H), 2.79-2.55 (m, 2H), 2.37 (m, 1H), 1.95-1.81 (m, 2H), 1.68-1.47 (m, 2H), 1.10-0.94 (m, 2H), 0.50-0.32 (m, 4H), 0.19-0.10 (m, 4H). Mass (m/z): 497.3 [M+H]⁺.

### Compound 398

### 5-((4-(4-(trifluoromethyl)piperidin-1-yl)phenyl)amino)-1H-indazole-1-carboxamide

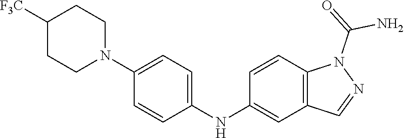 [SMILES: NC(=O)n1ncc2cc(Nc3ccc(N4CCC(C(F)(F)F)CC4)cc3)ccc21]

The title compound 398 (8.2 mg) was prepared in a yield of 9.9% as a white powder from 4-(4-(trifluoromethyl)piperidin-1-yl)aniline (50 mg, 0.20 mmol) and 5-bromo-1H-indazole-1-carboxamide (49 mg, 0.20 mmol) according to the procedure for compound 1. ¹H NMR (DMSO-d₆) δ 8.13 (d, J=0.8 Hz, 1H), 8.06 (d, J=9.0 Hz, 1H), 7.89 (s, 1H), 7.58 (d, J=45.8 Hz, 2H), 7.28-7.23 (m, 1H), 7.18 (dd, J=9.0, 2.2 Hz, 1H), 7.02 (d, J=8.9 Hz, 2H), 6.92 (d, J=8.5 Hz, 2H), 3.63 (d, J=12.2 Hz, 1H), 2.67 (q, J=1.9 Hz, 2H), 2.32 (d, J=1.9 Hz, 1H), 1.87 (s, 2H), 1.58 (dd, J=12.2, 4.0 Hz, 2H). Mass (m/z): 404.6 [M+H]⁺.

### Compound 399

### N3,N3,1-trimethyl-N4-(4-(4-(trifluoromethyl)piperidin-1-yl)phenyl)-1H-indole-3,4-diamine

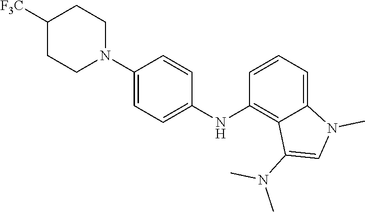 [SMILES: CN(C)c1cn(C)c2cccc(Nc3ccc(N4CCC(C(F)(F)F)CC4)cc3)c12]

The title compound 399 (34.3 mg) was prepared in a total yield of 41.6% as a white solid from 4-(4-(trifluoromethyl)piperidin-1-yl)aniline (58 mg, 0.237 mmol), 4-bromo-N,N,1-trimethyl-1H-indol-3-amine (50 mg, 0.198 mmol), Pd₂(dba)₃ (2 mg, 0.002 mmol), X-phos (6 mg, 0.01 mmol), Cs₂CO₃ (97 mg, 0.297 mmol) and 1,4-dioxane (5 mL) according to the procedure for compound 1. ¹H NMR (400 MHz, DMSO-d₆) δ 7.69 (s, 1H), 7.31-7.01 (m, 6H), 6.90 (s, 1H), 3.78 (s, 3H), 3.65 (d, J=12.0 Hz, 2H), 3.13 (s, 6H), 2.62 (s, 1H), 2.02 (d, J=13.6 Hz, 2H), 1.77 (d, J=12.4 Hz, 2H). Mass (m/z): 417.3 [M+H]⁺.

### Compound 400

### 7-methoxy-3-methyl-5-((4-(4-(trifluoromethyl)piperidin-1-yl)phenyl) amino)benzo[d]oxazol-2(3H)-one

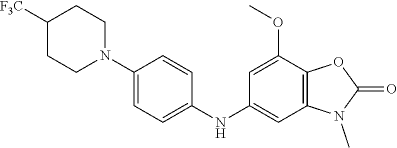 [SMILES: COc1cc(Nc2ccc(N3CCC(C(F)(F)F)CC3)cc2)cc2c1oc(=O)n2C]

The title compound 400 (49.1 mg) was prepared in a total yield of 60.1% as a white solid from 4-(4-(trifluoromethyl)piperidin-1-yl)aniline (57 mg, 0.232 mmol), 5-bromo-7-methoxy-3-methylbenzo[d]oxazol-2(3H)-one (50 mg, 0.194 mmol), Pd₂(dba)₃ (2 mg, 0.002 mmol), X-phos (6 mg, 0.01 mmol), Cs₂CO₃ (95 mg, 0.291 mmol) and 1,4-dioxane (5 mL) according to the procedure for compound 1. ¹H NMR (400 MHz, DMSO-d₆) δ 7.94 (s, 1H), 7.05-6.99 (m, 2H), 6.94-6.88 (m, 2H), 6.37 (s, 2H), 3.83 (s, 3H), 3.64 (d, J=12.4 Hz, 2H), 3.24 (s, 3H), 2.63 (dd, J=13.2, 10.7 Hz, 2H), 2.42 (td, J=8.8, 4.0 Hz, 1H), 1.93-1.82 (m, 2H), 1.57 (qd, J=12.4, 4.0 Hz, 2H). Mass (m/z): 422.3 [M+H]⁺.

### Compound 401

### N2-ethyl-1-methyl-N5-(4-(4-(trifluoromethyl)piperidin-1-yl)phenyl)-1H-benzo[d]imidazole-2,5-diamine

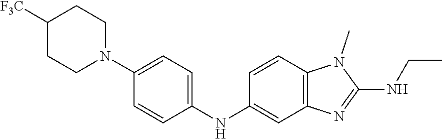 [SMILES: CCNc1nc2cc(Nc3ccc(N4CCC(C(F)(F)F)CC4)cc3)ccc2n1C]

The title compound 401 (23.6 mg) was prepared in a yield of 28.7% as a white powder from 4-(4-(trifluoromethyl)piperidin-1-yl)aniline (50 mg, 0.20 mmol) and 5-bromo-N-ethyl-1-methyl-1H-benzo[d]imidazol-2-amine (48 mg, 0.20 mmol) according to the procedure for compound 1. ¹H NMR (DMSO-d₆) δ 12.75 (s, 1H), 8.66 (s, 1H), 8.15 (s, 1H), 7.17 (d, J=87.5 Hz, 6H), 4.22 (s, 5H), 3.59 (d, J=40.2 Hz, 7H), 1.96 (s, 1H), 1.69 (s, 1H), 1.27 (t, J=7.2 Hz, 3H). Mass (m/z): 418.6 [M+H]⁺.

### Compound 402

### N2,N2-diethyl-1-methyl-N6-(4-(4-(trifluoromethyl)piperidin-1-yl)phenyl)-1H-benzo[d]imidazole-2,6-diamine

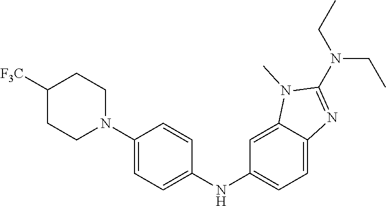 [SMILES: CCN(CC)c1nc2ccc(Nc3ccc(N4CCC(C(F)(F)F)CC4)cc3)cc2n1C]

The title compound 402 (28.5 mg) was prepared in a total yield of 36.1% as a white solid from 4-(4-(trifluoromethyl)piperidin-1-yl)aniline (52 mg, 0.213 mmol), 6-bromo-N,N-diethyl-1-methyl-1H-benzo[d]imidazol-2-amine (50 mg, 0.177 mmol), Pd₂(dba)₃ (2 mg, 0.002 mmol), X-phos (6 mg, 0.01 mmol), Cs₂CO₃ (87 mg, 0.265 mmol) and 1,4-dioxane (5 mL) according to the procedure for compound 1. ¹H NMR (400 MHz, Pyridine-d₅) δ 7.76 (s, 1H), 7.05 (s, 2H), 3.56 (s, 2H), 3.36 (s, 3H), 3.33-3.20 (m, 4H), 2.52 (s, 2H), 2.12 (s, 1H), 1.78 (s, 2H), 1.66 (d, J=12.8 Hz, 2H), 1.24 (d, J=6.4 Hz, 1H), 1.09 (t, J=7.2 Hz, 6H). Mass (m/z): 446.3 [M+H]⁺.

### Compound 403

### 1-methyl-6-((4-(4-(trifluoromethyl)piperidin-1-yl)phenyl)amino)-1H-indole-3-carboxamide

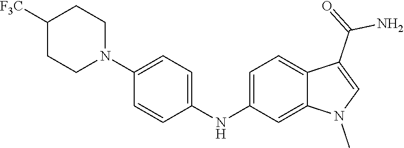 [SMILES: Cn1cc(C(N)=O)c2ccc(Nc3ccc(N4CCC(C(F)(F)F)CC4)cc3)cc21]

The title compound 403 (9.6 mg) was prepared in a total yield of 11.5% as a light yellow powder from 4-(4-(trifluoromethyl)piperidin-1-yl)aniline (63 mg, 0.26 mmol, 6-bromo-1-methyl-1H-indole-3-carboxamide (50 mg, 0.20 mmol), Pd₂(dba)₃ (2.3 mg, 2.5 umol), X-Phos (6.0 mg, 12.5 umol), and Cs₂CO₃ (122 mg, 0.38 mmol) according to the procedure for compound 1. ¹H NMR (400 MHz, DMSO-d₆) δ 7.91 (d, J=8.5 Hz, 1H), 7.76 (d, J=7.3 Hz, 2H), 7.22 (s, 1H), 7.05-7.01 (m, 2H), 6.96 (d, J=1.9 Hz, 1H), 6.92-6.86 (m, 2H), 6.82 (dd, J=8.7, 2.0 Hz, 1H), 6.68 (s, 1H), 3.67 (s, 3H), 3.64-3.58 (m, 2H), 2.69-2.59 (m, 2H), 2.46-2.38 (m, 1H), 1.92-1.85 (m, 2H), 1.64-1.50 (m, 2H). Mass (m/z): 417.2 [M+H]⁺.

### Compound 404

### 1,3-dimethyl-5-((4-(trifluoromethyl)phenyl)amino)-1,3-dihydro-2H-benzo [d]imidazol-2-one

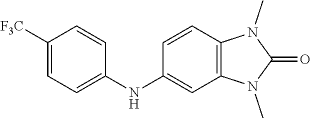 [SMILES: Cn1c(=O)n(C)c2cc(Nc3ccc(C(F)(F)F)cc3)ccc21]

The title compound 404 (90.3 mg) was prepared in a total yield of 67.4% as a white solid from 4-(trifluoromethyl)aniline (80 mg, 0.450 mmol), 5-bromo-1,3-dimethyl-1,3-dihydro-2H-benzo[d]imidazol-2-one (100 mg, 0.417 mmol), Pd₂(dba)₃ (4 mg, 0.004 mmol), X-phos (12 mg, 0.02 mmol), Cs₂CO₃ (200 mg, 0.625 mmol) and 1,4-dioxane (5 mL) according to the procedure for compound 1. ¹H NMR (400 MHz, DMSO-d₆) δ 8.65 (s, 1H), 7.46 (d, J=8.4 Hz, 2H), 7.11 (d, J=8.0 Hz, 1H), 7.08-6.98 (m, 3H), 6.90 (dd, J=8.4, 2.0 Hz, 1H), 3.31 (d, J=6.8 Hz, 6H). Mass (m/z): 322.3 [M+H]⁺.

### Compound 405

### 1-(2-(dimethylamino)ethyl)-5-((4-(4,4-dimethylcyclohexyl)phenyl)amino)-3-methyl-1,3-dihydro-2H-benzo[d]imidazol-2-one

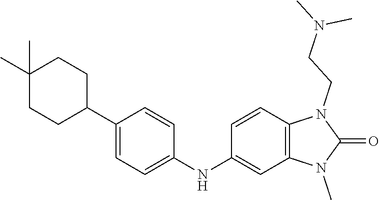 [SMILES: CN(C)CCn1c(=O)n(C)c2cc(Nc3ccc(C4CCC(C)(C)CC4)cc3)ccc21]

The title compound 405 (11.0 mg) was prepared in a total yield of 19.4% as a green solid from 4-(4,4-dimethylcyclohexyl)aniline (30 mg, 0.148 mmol), 5-bromo-1-(2-(dimethylamino)ethyl)-3-methyl-1,3-dihydro-2H-benzo[d]imidazol-2-one (40 mg, 0.135 mmol), Pd₂(dba)₃ (2 mg, 0.002 mmol), X-phos (6 mg, 0.01 mmol), Cs₂CO₃ (66 mg, 0.202 mmol) and 1,4-dioxane (5 mL) according to the procedure for compound 1. ¹H NMR (400 MHz, DMSO-d₆) δ 9.44 (s, 1H), 7.90 (s, 1H), 7.15 (d, J=8.4 Hz, 1H), 7.08 (d, J=8.4 Hz, 2H), 6.97-6.89 (m, 3H), 6.79 (dd, J=8.4, 2.0 Hz, 1H), 4.18 (t, J=6.0 Hz, 2H), 3.29 (s, 3H), 2.89 (s, 6H), 2.29 (dt, J=10.4, 5.2 Hz, 1H), 1.61-1.38 (m, 6H), 1.28 (ddd, J=23.2, 11.6, 4.4 Hz, 3H), 0.95 (d, J=9.6 Hz, 6H). Mass (m/z): 421.3 [M+H]⁺.

### Compound 406

### 6-((4-(4,4-dimethylcyclohexyl)phenyl)amino)-3-methylbenzo[d]oxazol-2(3H)-one

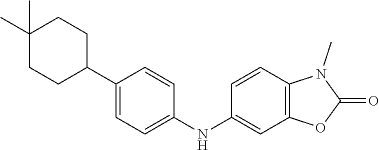 [SMILES: Cn1c(=O)oc2cc(Nc3ccc(C4CCC(C)(C)CC4)cc3)ccc21]

The title compound 406 (6.8 mg) was prepared in a total yield of 11.1% as a white solid from 4-(4,4-dimethylcyclohexyl)aniline (39 mg, 0.193 mmol), 6-bromo-3-methylbenzo[d]oxazol-2(3H)-one (40 mg, 0.175 mmol), Pd₂(dba)₃ (2 mg, 0.002 mmol), X-phos (6 mg, 0.01 mmol), Cs₂CO₃ (84 mg, 0.262 mmol) and 1,4-dioxane (5 mL) according to the procedure for compound 1. ¹H NMR (400 MHz, DMSO-d₆) δ 8.01 (s, 1H), 7.10 (dd, J=8.4, 2.0 Hz, 3H), 7.00 (d, J=2.0 Hz, 1H), 6.96-6.92 (m, 2H), 6.89 (dd, J=8.4, 2.0 Hz, 1H), 3.30 (s, 3H), 2.31 (tt, J=10.4, 5.2 Hz, 1H), 1.60-1.54 (m, 3H), 1.48-1.41 (m, 2H), 1.35-1.21 (m, 3H), 0.95 (d, J=10.0 Hz, 6H). Mass (m/z): 351.3 [M+H]⁺.

### Compound 407

### 5-((4-(4,4-dimethylcyclohexyl)phenyl)amino)-1,3-dimethyl-1,3-dihydro-2H-benzo[d]imidazol-2-one

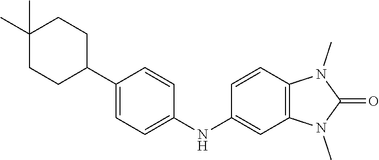 [SMILES: Cn1c(=O)n(C)c2cc(Nc3ccc(C4CCC(C)(C)CC4)cc3)ccc21]

The title compound 407 (4.4 mg) was prepared in a total yield of 7.2% as a green solid from 4-(4,4-dimethylcyclohexyl)aniline (37 mg, 0.183 mmol), 5-bromo-1,3-dimethyl-1,3-dihydro-2H-benzo[d]imidazol-2-one (40 mg, 0.167 mmol), Pd₂(dba)₃ (2 mg, 0.002 mmol), X-phos (6 mg, 0.01 mmol), Cs₂CO₃ (80 mg, 0.250 mmol) and 1,4-dioxane (5 mL) according to the procedure for compound 1. ¹H NMR (400 MHz, DMSO-d₆) δ 7.83 (s, 1H), 7.09-7.03 (m, 2H), 7.00 (d, J=8.4 Hz, 1H), 6.94-6.89 (m, 2H), 6.85 (d, J=2.0 Hz, 1H), 6.77 (dd, J=8.4, 2.0 Hz, 1H), 3.28 (d, J=8.0 Hz, 6H), 2.28 (dt, J=10.4, 5.2 Hz, 1H), 1.61-1.54 (m, 3H), 1.44 (d, J=12.8 Hz, 2H), 1.29 (td, J=12.8, 6.0 Hz, 3H), 0.94 (d, J=10.0 Hz, 6H). Mass (m/z): 364.3 [M+H]⁺.

### Compound 408

### 6-(4-(4,4-dimethylcyclohexyl)phenoxy)benzo[d]thiazol-2-amine

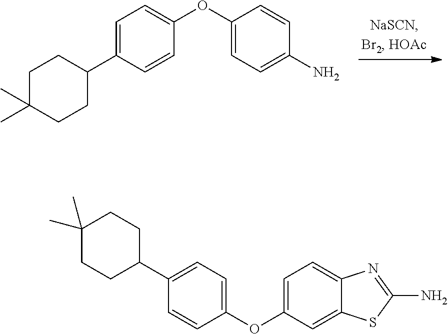 [SMILES: CC1(C)CCC(c2ccc(Oc3ccc(N)cc3)cc2)CC1 ; CC1(C)CCC(c2ccc(Oc3ccc4nc(N)sc4c3)cc2)CC1]

A solution of 4-(4-(4,4-dimethylcyclohexyl)phenoxy)aniline (50 mg, 0.17 mmol), NaSCN (16.5 mg, 0.20 mmol) and Br₂ (32 mg, 0.20 mmol) in HOAc (1 mL) was stirred at r.t. for 18 hours. After filtration, the filtrate was concentrated under vacuum. The residue was purified by prep-HPLC (column-Xbridge-C18 150×19 mm, 5 um; Mobile phase: ACN-H₂O (0.1% FA), 25%-40%) to afford 408 (22.3 mg) as a white solid. Mass (m/z): 353.2 [M+H]⁺. ¹H NMR (400 MHz, DMSO-d⁶) δ 7.43 (d, J=2.6 Hz, 1H), 7.34 (d, J=8.8 Hz, 1H), 7.22-7.19 (m, 2H), 6.96 (dd, J=8.8, 2.5 Hz, 1H), 6.87-6.84 (m, 2H), 2.36 (td, J=10.6, 4.9 Hz, 1H), 1.54 (dt, J=16.4, 5.9 Hz, 4H), 1.41 (d, J=12.4 Hz, 2H), 1.31-1.24 (m, 2H), 0.91 (d, J=10.0 Hz, 6H).

### Compound 409

### N⁶-(4-(4,4-dimethylcyclohexyl)phenyl)benzo[d]thiazole-2,6-diamine

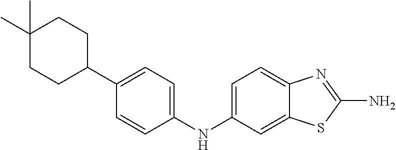 [SMILES: CC1(C)CCC(c2ccc(Nc3ccc4nc(N)sc4c3)cc2)CC1]

A solution of N¹-(4-(4,4-dimethylcyclohexyl)phenyl)benzene-1,4-diamine (60 mg, 0.20 mmol), NaSCN (20.0 mg, 0.24 mmol) and Br₂ (39.0 mg, 0.24 mmol) in MeOH (2 mL) was stirred at r.t. for 18 hours. The solids were filtered, the filtrate was concentrated under vacuum. The residue was purified by prep-HPLC (column-Xbridge-C18 150×19 mm, 5 um; Mobile phase: ACN-H₂O (0.1% FA), 25%-40%) to afford compound 409 (5.0 mg) as a white solid. Mass (m/z): 352.2 [M+H]⁺. ¹H NMR (400 MHz, CD₃OD) δ 7.29 (d, J=2.2 Hz, 1H), 7.24 (d, J=8.6 Hz, 1H), 7.07 (d, J=8.4 Hz, 2H), 6.98 (dd, J=8.6, 2.4 Hz, 1H), 6.95 (d, J=8.6 Hz, 2H), 2.38-2.31 (m, 1H), 1.64 (d, J=3.0 Hz, 2H), 1.50 (d, J=12.0 Hz, 2H), 1.33 (dd, J=20.0, 6.8 Hz, 4H), 1.00 (s, 3H), 0.95 (s, 3H).

### Compound 410

### N-(2-methyl-4-(4-(trifluoromethyl)piperidin-1-yl)phenyl) isoindolin-5-amine

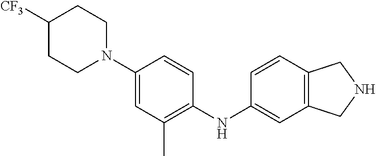 [SMILES: Cc1cc(N2CCC(C(F)(F)F)CC2)ccc1Nc1ccc2c(c1)CNC2]

To a solution of 2-methyl-4-(4-(trifluoromethyl)piperidin-1-yl)aniline (72 mg, 0.278 mmol) and 5-bromoisoindoline (50 mg, 0.253 mmol) in 1,4-dioxane (5 mL) was added BrettPhos Pd G3 (23 mg, 0.025 mmol), BrettPhos (27 mg, 0.051 mmol) and Cs₂CO₃ (247 mg, 0.759 mmol), then the mixture was stirred at 110° C. for 16 h. The mixture was extracted by EA (25 mL×3). The combined organic layers were washed with brine (15 mL×3), dried over Na₂SO₄ and concentrated to give the crude product, which was purified by TLC (MeOH/DCM=1/5) to give the desired product as a yellow solid (14.2 mg, 14.9%). ¹H NMR (400 MHz, DMSO-d₆) δ 6.97 (t, J=8.0 Hz, 2H), 6.84 (s, 1H), 6.75 (d, J=9.2 Hz, 1H), 6.51 (d, J=6.4 Hz, 2H), 3.92 (d, J=6.0 Hz, 3H), 3.68 (d, J=12.4 Hz, 2H), 2.66 (q, J=12.4 Hz, 3H), 2.44 (d, J=10.0 Hz, 1H), 2.12 (s, 3H), 1.88 (d, J=12.4 Hz, 2H), 1.56 (q, J=12.4 Hz, 2H). Mass (m/z): 376.3 [M+H]⁺.

### Compound 411

### 1,3-dimethyl-5-((4-(tert-pentyl)phenyl)amino)-1,3-dihydro-2H-benzo[d]imidazol-2-one

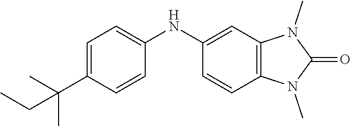 [SMILES: CCC(C)(C)c1ccc(Nc2ccc3c(c2)n(C)c(=O)n3C)cc1]

To a solution of 5-bromo-1,3-dimethyl-1,3-dihydro-2H-benzo[d]imidazol-2-one (134 mg, 0.558 mmol) and 4-(tert-pentyl)aniline (100 mg, 0.613 mmol) in dioxane (5 mL) was added Pd₂(dba)₃ (25.5 mg, 0.028 mmol), X-phos (26.6 mg, 0.0558 mmol) and Cs₂CO₃ (364 mg, 1.12 mmol) under nitrogen atmosphere. Then the mixture was stirred at 100° C. overnight. The mixture was extracted by EA (25 mL×3). The combined organic layers were washed with brine (15 mL×3), dried over Na₂SO₄ and concentrated to give the crude product, which was purified by pre-HPLC to give the desired product as a white solid (20.3 mg, 24.0%). H NMR (300 MHz, DMSO-d₆) δ 7.82 (s, 1H), 7.13 (d, J=8.6 Hz, 2H), 6.99 (d, J=8.3 Hz, 1H), 6.92 (d, J=8.6 Hz, 2H), 6.85 (d, J=2.0 Hz, 1H), 6.77 (dd, J=8.3, 2.0 Hz, 1H), 3.28 (d, J=4.9 Hz, 6H), 1.55 (t, J=7.4 Hz, 2H), 1.20 (s, 6H), 0.64 (t, J=7.4 Hz, 3H). Mass (m/z): 324.2 [M+H]⁺

### Compound 412

### 3-(5-((4-(4-(trifluoromethyl)piperidin-1-yl)phenyl)amino)-1H-indazol-1-yl)propanamide

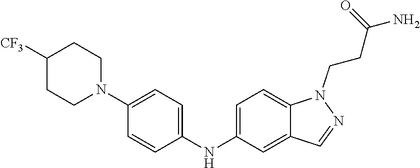 [SMILES: NC(=O)CCn1ncc2cc(Nc3ccc(N4CCC(C(F)(F)F)CC4)cc3)ccc21]

The title compound 412 (37.7 mg) was prepared in a total yield of 36.5% as a purple powder from 4-(4-(trifluoromethyl)piperidin-1-yl)aniline (70 mg, 0.29 mmol), 3-(5-bromo-1H-indazol-1-yl)propanamide (63 mg, 0.24 mmol), Pd₂(dba)₃ (2.3 mg, 2.5 umol), X-Phos (6.0 mg, 12.5 umol), and Cs₂CO₃ (122 mg, 0.38 mmol) according to the procedure for compound 1. ¹H NMR (400 MHz, Methanol-d₄) δ 7.81 (s, 1H), 7.55 (s, 2H), 7.32-6.68 (m, 7H), 4.63 (t, J=6.8 Hz, 2H), 3.75-3.48 (m, 2H), 2.79 (t, J=6.8 Hz, 2H), 2.74-2.47 (m, 2H), 2.35-2.22 (m, 1H), 2.04-1.88 (m, 2H), 1.80-1.64 (m, 2H). Mass (m/z): 432.1 [M+H]⁺.

### Compound 413

### 3-(5-((4-(4-(trifluoromethyl)piperidin-1-yl)phenyl)amino)-2H-indazol-2-yl)propanamide

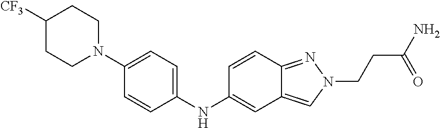 [SMILES: NC(=O)CCn1cc2cc(Nc3ccc(N4CCC(C(F)(F)F)CC4)cc3)ccc2n1]

The title compound 413 (16.5 mg) was prepared in a total yield of 16.0% as a purple powder from 4-(4-(trifluoromethyl)piperidin-1-yl)aniline (70 mg, 0.29 mmol), 3-(5-bromo-2H-indazol-2-yl)propanamide (63 mg, 0.24 mmol), Pd₂(dba)₃ (2.3 mg, 2.5 umol), X-Phos (6.0 mg, 12.5 umol), and Cs₂CO₃ (122 mg, 0.38 mmol) according to the procedure for compound 1. ¹H NMR (400 MHz, Methanol-d₄) δ 7.91 (s, 2H), 7.46 (d, J=9.4 Hz, 1H), 7.30-6.71 (m, 7H), 4.63 (t, J=6.8 Hz, 2H), 3.75-3.48 (m, 2H), 2.89 (t, J=6.8 Hz, 2H), 2.80-2.61 (m, 2H), 2.35-2.22 (m, 1H), 2.04-1.88 (m, 2H), 1.80-1.64 (m, 2H). Mass (m/z): 432.1 [M+H]⁺.

### Compound 414

### 1-(3-(methylamino)propyl)-N-(4-(4-(trifluoromethyl)piperidin-1-yl) phenyl)-1H-indazol-5-amine

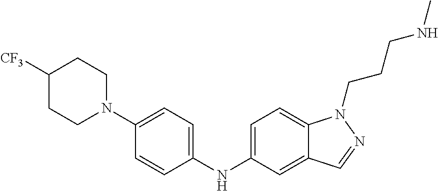 [SMILES: CNCCCn1ncc2cc(Nc3ccc(N4CCC(C(F)(F)F)CC4)cc3)ccc21]

To a solution of tert-butyl (3-(5-((4-(4-(trifluoromethyl)piperidin-1-yl) phenyl)amino)-1H-indazol-1-yl)propyl)carbamate (116 mg, 0.22 mol) in THF (10 mL) was added LiAlH₄ (17 mg, 0.45 mol) at 0° C., and the mixture was refluxed for 2 h. After cooling to 0° C., water (17 μL), 10% NaOH (34 μL) and water (51 μL) were added, and the mixture was stirred for 3 min at room temperature. The solid was filtered and the filtered gray cake was washed with THF (10 mL×2); then the combined filtrates were dried over Na₂SO₄ and concentrated. The residue was purified by prep-TLC (MeOH/DCM=1/5) to give the desired products as a white solid. (28.5 mg, 29.5%). ¹H NMR (400 MHz, DMSO-D₆) δ 8.67 (s, 1H), 7.83 (s, 1H), 7.68 (s, 1H), 7.53 (d, J=8.9 Hz, 1H), 7.19 (s, 1H), 7.08 (dd, J=9.1, 2.0 Hz, 1H), 6.93 (d, J=8.7 Hz, 2H), 6.86 (d, J=8.6 Hz, 2H), 4.40 (t, J=6.7 Hz, 2H), 3.56 (d, J=11.7 Hz, 2H), 2.91-2.79 (m, 2H), 2.62-2.55 (m, 2H), 2.39 (s, 1H), 2.10 (p, J=6.9 Hz, 2H), 1.85 (d, J=12.5 Hz, 2H), 1.67-1.44 (m, 2H). Mass (m/z): 432.3 [M+H]⁺.

### Compound 415

### 2-(3-(methylamino)propyl)-N-(4-(4-(trifluoromethyl)piperidin-1-yl) phenyl)-2H-indazol-5-amine

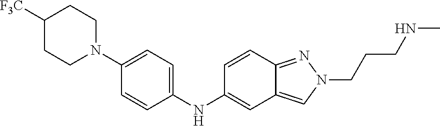 [SMILES: CNCCCn1cc2cc(Nc3ccc(N4CCC(C(F)(F)F)CC4)cc3)ccc2n1]

The title compound 415 (129 mg) was prepared in a total yield of 54.6% as a yellow powder from tert-butyl (3-(5-((4-(4-(trifluoromethyl)piperidin-1-yl)phenyl)amino)-2H-indazol-2-yl)propyl)carbamate (176 mg, 0.5 mmol) and LiAlH₄ (38 mg, 0.45 mol) according to the procedure for compound 414. ¹H NMR (400 MHz, DMSO-d₆) δ 8.92 (s, 2H), 8.06 (s, 1H), 7.67 (s, 1H), 7.44 (d, J=9.2 Hz, 1H), 7.05-6.88 (m, 5H), 4.42 (t, J=6.7 Hz, 2H), 3.57 (d, J=12.0 Hz, 2H), 2.92-2.75 (m, 2H), 2.62-2.54 (m, 2H), 2.45-2.32 (m, 1H) 2.27-2.14 (m, 2H), 1.92-1.80 (m, 2H), 1.62-1.47 (m, 2H). Mass (m/z): 432.3 [M+H]⁺.

### Compound 416

### tert-butyl(3-(5-((4-(2,6-dimethylmorpholino)phenyl)amino)-2-oxobenzo[d]oxazol-3(2H)-yl)propyl)((methyl)carbamate

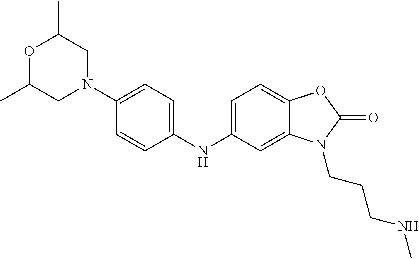

TFA (1 mL) was added to a solution of tert-butyl (3-(5-((4-(2,6-dimethylmorpholino)phenyl)amino)-2-oxobenzo[d]oxazol-3(2H)-yl)pr opyl)(methyl)carbamate (130 mg, 0.25 mmol) in dichloromethane (5 mL). The mixture was stirred at room temperature for 2 h. The reaction mixture was concentrated under vacuum. The residue was diluted with a saturated solution of NaHCO₃ (30 mL) and extracted with dichloromethane (3×50 mL). The combined organic phases were dried and concentrated. The residue was purified by Prep-HPLC with the following conditions: Column: Spherical C18 40-60 um, 40 g; Mobile phase A: water (0.5% NH₄HCO₃); Mobile phase B: acetonitrile or ACN; Flow rate: 45 mL/min; Gradient: 30% B-65% B in 20 min; Detector: 254 nm. The fractions containing desired product were collected at 55% B and concentrated under reduced pressure to give the titled compound 416 (51 mg, 48.8%) as a grey solid. LC-MS (m/z) 411.2 [M+H]⁺. ¹H NMR (400 MHz, DMSO-d₆) δ 8.66 (s, 1H), 7.87 (s, 1H), 7.15 (d, J=8.6 Hz, 1H), 7.03-6.97 (m, 2H), 6.93-6.84 (m, 3H), 6.64 (dd, J=8.6, 2.2 Hz, 1H), 3.84 (t, J=6.7 Hz, 2H), 3.69 (dtt, J=12.6, 6.4, 3.7 Hz, 2H), 3.48-3.40 (m, 2H), 3.31 (s, 3H), 2.95-2.86 (m, 2H), 2.19 (dd, J=11.8, 10.2 Hz, 2H), 1.99 (p, J=6.9 Hz, 2H), 1.14 (d, J=6.2 Hz, 6H).

### Compound 417

### 1-(2-(2-oxo-5-((4-(4-(trifluoromethyl)piperidin-1-yl)phenyl)amino) benzo[d]oxazol-3(2H)-yl)ethyl)urea

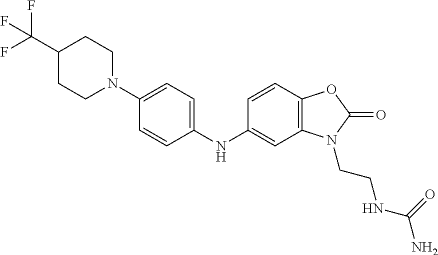 [SMILES: NC(=O)NCCn1c(=O)oc2ccc(Nc3ccc(N4CCC(C(F)(F)F)CC4)cc3)cc21]

To a solution of 3-(2-aminoethyl)-5-((4-(4-(trifluoromethyl) piperidin-1-yl)phenyl)amino)benzo[d]oxazol-2(3H)-one (crude) in DMSO (6 mL) was added phenyl carbamate (8 mg, 0.0577 mmol), TEA (7.5 mg, 0.0722 mmol). Then the mixture was stirred at 60° C. for 2 hours. The mixture was diluted with water and extracted by EA (25 mL×3). The combined organic layers were washed with brine (15 mL×3), dried over Na₂SO₄ and concentrated to give the crude product, which was purified by pre-HPLC to give the desired product as a blue solid (17.2 mg, 77%). ¹H NMR (400 MHz, DMSO-d₆) δ 7.79 (d, J=11.5 Hz, 1H), 7.08 (d, J=8.6 Hz, 1H), 6.99-6.92 (m, 2H), 6.89-6.84 (m, 2H), 6.76 (t, J=9.0 Hz, 1H), 6.58 (dt, J=11.3, 5.7 Hz, 1H), 6.17-6.03 (m, 1H), 5.41 (d, J=35.3 Hz, 2H), 3.76-3.65 (m, 2H), 3.56 (t, J=15.9 Hz, 2H), 3.21 (dd, J=15.1, 9.1 Hz, 2H), 1.85 (d, J=12.1 Hz, 3H), 1.63-1.47 (m, 3H). Mass (m/z): 464.2 [M+H]⁺.

### Compound 418

### 3-(3-(methylamino)propyl)-5-((4-(4-(trifluoromethyl)piperidin-1-yl) phenyl)amino)benzo[d]oxazol-2(3H)-one

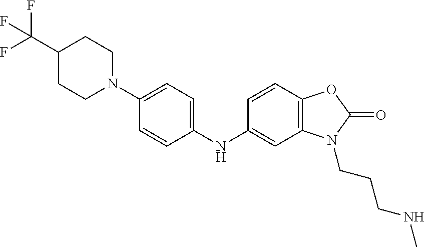 [SMILES: CNCCCn1c(=O)oc2ccc(Nc3ccc(N4CCC(C(F)(F)F)CC4)cc3)cc21]

To a solution of tert-butyl methyl(3-(2-oxo-5-((4-(4-(trifluoromethyl) piperidin-1-yl)phenyl)amino)benzo[d]oxazol-3(2H)-yl)propyl)carbamate (crude) in DCM (6 mL) was added TFA (2 mL). The mixture was stirred at r.t. for 0.5 hour. Then the mixture was concentrated to give the crude product, which was purified by pre-HPLC to give the desired product as a purple solid (13.9 mg, 12.6%). ¹H NMR (400 MHz, DMSO-d₆) δ 7.87 (s, 1H), 7.11 (d, J=8.6 Hz, 1H), 6.96 (d, J=9.0 Hz, 2H), 6.89-6.80 (m, 3H), 6.62 (dd, J=8.7, 2.2 Hz, 1H), 3.81 (t, J=6.8 Hz, 2H), 3.69-3.51 (m, 2H), 2.94-2.77 (m, 2H), 2.76-2.56 (m, 3H), 2.43 (s, 3H), 2.06-1.73 (m, 4H), 1.54 (qd, J=12.4, 4.0 Hz, 2H). Mass (m/z): 449.2 [M+H]⁺.

### Compound 419

### 1-(2-(5-((4-(4-(trifluoromethyl)piperidin-1-yl)phenyl)amino)-2H-indazol-2-yl)ethyl)urea

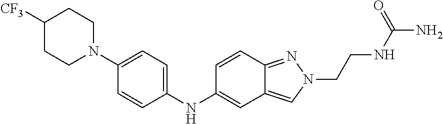 [SMILES: NC(=O)NCCn1cc2cc(Nc3ccc(N4CCC(C(F)(F)F)CC4)cc3)ccc2n1]

To a solution of 2-(2-aminoethyl)-N-(4-(4-(trifluoromethyl)piperidin-1-yl)phenyl)-2H-indazol-5-amine (66 mg, 0.15 mmol) and TEA (45 mg, 0.45 mmol) in DMSO (1 mL) was added phenyl carbamate (21 mg, 0.15 mmol). Then the mixture was stirred for 1 hour at 60° C. After cooling to r.t., 10 mL of water was added. The resulting solution was extracted with 3×10 mL of ethyl acetate. The organic layers were combined, washed with water (3×15 mL), dried and concentrated under vacuum. The residue was purified by prep-TLC (MeOH/DCM=1/10) to afford the desired product as a yellow solid (10.0 mg, 15.0%). ¹H NMR (400 MHz, DMSO-d₆) δ 7.96 (d, J=1.0 Hz, 1H), 7.61 (s, 1H), 7.47-7.40 (m, 1H), 7.05-7.02 (m, 1H), 6.97-6.93 (m, 2H), 6.88-6.84 (m, 1H), 5.96 (t, J=5.9 Hz, 1H), 5.48 (s, 2H), 4.30 (t, J=6.0 Hz, 2H), 3.57 (d, J=12.3 Hz, 2H), 3.45 (q, J=6.0 Hz, 2H), 2.63-2.56 (m, 2H), 2.44-2.33 (m, 1H), 1.88-1.80 (m, 2H), 1.62-1.47 (m, 1H). Mass (m/z): 447.2 [M+H]⁺.

### Compound 420

### 5-((4-(2,6-dimethylmorpholino)phenyl)amino)-3-methylbenzo[d]oxazol-2(3H)-one

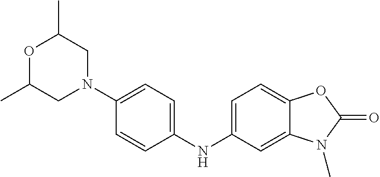 [SMILES: CC1CN(c2ccc(Nc3ccc4oc(=O)n(C)c4c3)cc2)CC(C)O1]

The titled compound 420 (62.3 mg, 30.9%) was obtained as an off-white solid according to the procedure outlined for compound 1. LC-MS (m/z) 354.2 [M+H]⁺. ¹H NMR (400 MHz, DMSO-d₆) δ 7.83 (s, 1H), 7.12 (d, J=8.6 Hz, 1H), 7.04-6.96 (m, 2H), 6.94-6.84 (m, 2H), 6.74 (d, J=2.3 Hz, 1H), 6.62 (dd, J=8.6, 2.3 Hz, 1H), 3.69 (dqd, J=12.4, 6.2, 2.2 Hz, 2H), 3.45 (dt, J=10.8, 2.0 Hz, 2H), 3.26 (s, 3H), 2.19 (dd, J=11.8, 10.2 Hz, 2H), 1.14 (d, J=6.2 Hz, 6H).

### Compound 421

### 1-(2-(5-((4-(4-(trifluoromethyl)piperidin-1-yl)phenyl)amino)-1H-indazol-1-yl)ethyl)urea

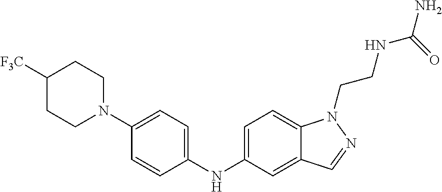 [SMILES: NC(=O)NCCn1ncc2cc(Nc3ccc(N4CCC(C(F)(F)F)CC4)cc3)ccc21]

The title compound 421 (19.9 mg) was prepared in a total yield of 29.9% as a light yellow powder from 1-(2-aminoethyl)-N-(4-(4-(trifluoromethyl) piperidin-1-yl)phenyl)-1H-indazol-5-amine (66 mg, 0.15 mmol), TEA (48 mg, 0.45 mmol) and phenyl carbamate (21 mg, 0.15 mmol) according to the procedure for compound 1. ¹H NMR (400 MHz, Methanol-d₄) δ 7.84 (s, 1H), 7.50-6.65 (m, 8H), 4.44 (t, J=6.0 Hz, 2H), 3.75-3.52 (m, 4H), 2.78-2.53 (m, 2H), 2.34-2.20 (m, 2H), 2.05-1.92 (m, 2H), 1.82-1.66 (m, 2H). Mass (m/z): 447.3 [M+H]⁺.

### Compound 422

### N-(4-(2,6-dimethylmorpholino)phenyl)-1-methyl-1H-indazol-5-amine

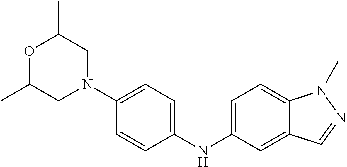 [SMILES: CC1CN(c2ccc(Nc3ccc4c(cnn4C)c3)cc2)CC(C)O1]

The titled compound 422 (89.6 mg, 30.9%) as a gray solid was prepared according to the procedure outlined for compound 1. LC-MS (m/z) 337.2 [M+H]⁺. ¹H NMR (400 MHz, DMSO-d₆) δ 8.07 (s, 2H), 7.51 (s, 1H), 7.18 (s, 3H), 7.02 (s, 3H), 4.10 (s, 3H), 3.84 (s, 2H), 3.48 (s, 2H), 2.75-2.60 (m, 2H), 1.17 (d, J=6.2 Hz, 6H).

### Compound 423

### N-(4-(2,6-dimethylmorpholino)phenyl)-2-methyl-2H-indazol-5-amine

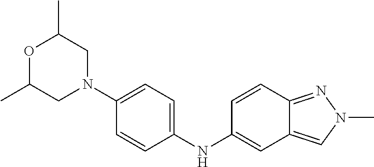

The titled compound 423 (68.1 mg, 30.9%) as a light yellow solid was prepared according to the procedure outlined for compound 1. LC-MS (m/z) 337.2 [M+H]⁺. ¹H NMR (400 MHz, DMSO-d₆) δ 8.09 (s, 2H), 7.50 (s, 1H), 7.20 (s, 3H), 7.02 (s, 3H), 4.05 (s, 3H), 3.81 (s, 2H), 3.45 (s, 2H), 2.74-2.60 (m, 2H), 1.17 (d, J=6.2 Hz, 6H).

### Compound 424

### 3-(2-(dimethylamino)ethyl)-6-((4-(2,6-dimethylmorpholino)phenyl) amino)benzo[d]oxazol-2(3H))-one

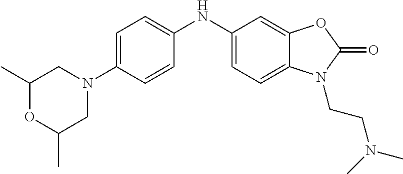

To a solution of 5-bromo-3-(2-(dimethylamino)ethyl)benzo[d]oxazol-2(3H)-one (96.5 mg, 0.339 mmol) and 4-(2,6-dimethylmorpholino)aniline (70 mg, 0.339 mmol) in dioxane (5 mL) was added Pd₂(dba)₃ (15.6 mg, 0.017 mmol), X-phos (16.2 mg, 0.0339 mmol) and Cs₂CO₃ (166.2 mg, 0.51 mmol) under nitrogen atmosphere. Then the mixture was stirred at 100° C. overnight. The mixture was extracted by EA (25 mL×3). The combined organic layers were washed with brine (15 mL×3), dried over Na₂SO₄ and concentrated to give the crude product, which was purified by pre-HPLC to give the desired product as a pink solid (35.2 mg, 25.3%). ¹H NMR (400 MHz, DMSO-d₆) δ 7.81 (s, 1H), 6.99 (dd, J=15.6, 8.9 Hz, 3H), 6.88 (d, J=9.0 Hz, 2H), 6.61 (dd, J=8.7, 2.4 Hz, 1H), 6.53 (d, J=2.4 Hz, 1H), 5.76 (s, 1H), 4.54 (s, 2H), 3.93 (t, J=6.9 Hz, 2H), 3.75-3.62 (m, 3H), 3.45 (d, J=10.6 Hz, 2H), 2.39 (t, J=6.9 Hz, 2H), 2.23-2.12 (m, 7H), 2.06 (s, 1H), 1.15 (t, J=5.7 Hz, 6H). Mass (m/z): 411.2 [M+H]⁺.

### Compound 425

### 5-((4-(2,6-dimethylmorpholino)phenyl)amino)-1-methyl-1H-indole-3-carboxamide

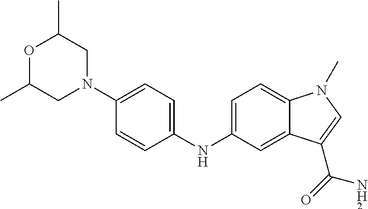 [SMILES: CC1CN(c2ccc(Nc3ccc4c(c3)c(C(N)=O)cn4C)cc2)CC(C)O1]

To a solution of 5-((4-(2,6-dimethylmorpholino)phenyl)amino)-1-methyl-1H-indole-3-carboxylic acid (120 mg, 0.317 mmol) in DMF (5 mL) was added HATU (301 mg, 0.793 mmol), NH₄Cl (33.9 mg, 0.633 mmol) and TEA (128 mg, 1.27 mmol). The mixture was stirred at 80° C. for 2 hours. Then the mixture was diluted with water and extracted by EA (25 mL×3). The combined organic layers were washed with brine (15 mL×3), dried over Na₂SO₄ and concentrated to give the crude product, which was purified by pre-HPLC to give the desired product as green solid (9.2 mg, 7.7%). ¹H NMR (400 MHz, DMSO-d₆) δ 7.92-7.76 (m, 2H), 7.57 (s, 1H), 7.32 (d, J=8.8 Hz, 1H), 6.97-6.88 (m, 3H), 6.84 (d, J=9.0 Hz, 2H), 3.75 (s, 3H), 3.72-3.63 (m, 2H), 3.45-3.36 (m, 2H), 2.17 (t, J=11.0 Hz, 2H), 1.14 (d, J=6.2 Hz, 6H). Mass (m/z): 379.2 [M+H]⁺.

### Compound 426

### 1-(2-(dimethylamino)ethyl)-5-((4-(2,6-dimethylmorpholino)phenyl) amino)-3-methyl-1,3-dihydro-2H-benzo[d]imidazol-2-one

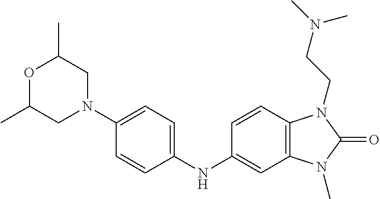 [SMILES: CC1CN(c2ccc(Nc3ccc4c(c3)n(C)c(=O)n4CCN(C)C)cc2)CC(C)O1]

The titled compound 426 (16.7 mg, 8%) as a purple solid was prepared from 4-(2,6-dimethylmorpholino)aniline (100 mg, 0.50 mmol), 5-bromo-1-(2-(dimethylamino)ethyl)-3-methyl-1H-benzo[d]imidazol-2(3H )-one (150 mg, 0.51 mmol), 1,4-dioxane (2 mL), Pd₂(dba)₃ (46 mg, 0.05 mmol), Xphos (48 mg, 0.10 mmol) and Cs₂CO₃ (329 mg, 1.01 mmol) according to the procedure outlined for compound 1. ¹H NMR (400 MHz, DMSO-d₆) δ 7.61 (s, 2H), 7.00 (d, J=8.3 Hz, 2H), 6.98-6.92 (m, 4H), 6.89-6.81 (m, 4H), 6.73 (d, J=2.1 Hz, 2H), 6.66 (dd, J=8.4, 2.1 Hz, 2H), 3.85 (t, J=6.6 Hz, 4H), 3.75-3.63 (m, 4H), 3.45-3.37 (m, 4H), 3.24 (s, 6H), 2.48 (s, 1H), 2.20 (s, 1H), 2.17 (s, 14H), 2.15 (s, 1H), 1.14 (d, J=6.2 Hz, 12H). Mass (m/z): 424.4 [M+H]⁺.

### Compound 427

### 5-((4-(2,6-dimethylmorpholino)phenyl)amino)-3-(2-(methylamino)ethyl) benzo[d]oxazol-2(3H)-one

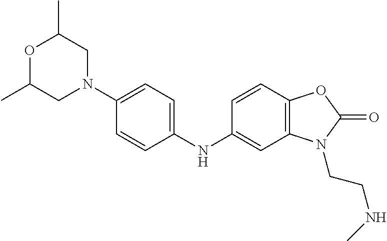 [SMILES: CNCCn1c(=O)oc2ccc(Nc3ccc(N4CC(C)OC(C)C4)cc3)cc21]

To a solution of tert-butyl (2-(5-((4-(2,6-dimethylmorpholino)phenyl) amino)-2-oxobenzo[d]oxazol-3(2H)-yl)ethyl)(methyl)carbamate (crude) in DCM (6 mL) was added TFA (2 mL). The mixture was stirred at r.t. for 0.5 hour. Then the mixture was concentrated to give the crude product, which was purified by pre-HPLC to give the desired product as gray solid (7.4 mg, 4.8%). ¹H NMR (400 MHz, DMSO-d₆) δ 7.57 (s, 1H), 6.97-6.87 (m, 4H), 6.86-6.75 (m, 3H), 6.58 (s, 1H), 3.77-3.60 (m, 7H), 3.39 (dd, J=18.6, 9.6 Hz, 3H), 2.52 (d, J=2.8 Hz, 2H), 2.20-2.10 (m, 2H), 1.13 (d, J=6.2 Hz, 6H). Mass (m/z): 397.2 [M+H]⁺.

### Compound 428

### 5-((4-(2,6-dimethylmorpholino)phenyl)amino)-1,3,3-trimethylindolin-2-one

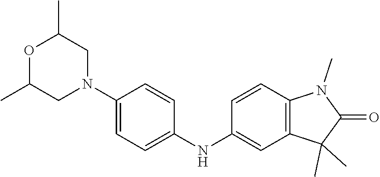 [SMILES: CC1CN(c2ccc(Nc3ccc4c(c3)C(C)(C)C(=O)N4C)cc2)CC(C)O1]

To a solution of 5-bromo-1,3,3-trimethylindolin-2-one (135 mg, 0.53 mmol) and 4-(2,6-dimethylmorpholino)aniline (100 mg, 0.49 mmol) in 1,4-dioxane (2 mL) was added Pd₂(dba)₃ (44 mg, 0.05 mmol), Xphos (46 mg, 0.09 mmol) and Cs₂CO₃ (317 mg, 0.97 mmol) under nitrogen atmosphere. The mixture was stirred at 100° C. for 12 h, the mixture was purified by prep HPLC to give 5-((4-(2,6-dimethylmorpholino) phenyl)amino)-1,3,3-trimethylindolin-2-one (71 mg, 38%) as a light purple solid. Mass (m/z): 380.4 [M+H]⁺. ¹H NMR (400 MHz, DMSO-d₆) δ 7.57 (s, 1H), 6.95-6.91 (m, 1H), 6.89-6.85 (m, 2H), 6.83-6.79 (m, 4H), 3.65 (s, 2H), 3.41-3.34 (m, 2H), 2.16-2.11 (m, 2H), 1.20 (s, 6H), 1.10 (d, J=6.2 Hz, 6H).

### Compound 429

### 3-(2-(dimethylamino)ethyl)-5-((4-(4-(trifluoromethyl)piperidin-1-yl) phenyl)amino)benzo[d]oxazol-2(3H)-one

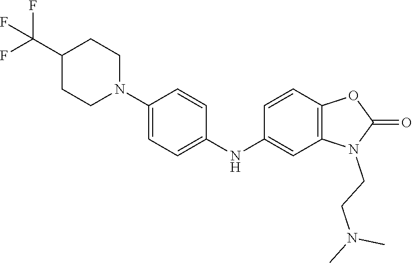 [SMILES: CN(C)CCn1c(=O)oc2ccc(Nc3ccc(N4CCC(C(F)(F)F)CC4)cc3)cc21]

To a solution of 5-bromo-3-(2-(dimethylamino)ethyl)benzo[d]oxazol-2(3H)-one (70 mg, 0.25 mmol) and 5-bromo-2-methylisoindoline (66 mg, 0.27 mmol) in 1,4-dioxane (2 mL) was added Pd₂(dba)₃ (22 mg, 0.02 mmol), Xphos (23 mg, 0.04 mmol) and Cs₂CO₃ (161 mg, 0.50 mmol) under nitrogen atmosphere. The mixture was stirred at 100° C. for 12 h and the mixture was purified by flash chromatography to give 3-(2-(dimethylamino)ethyl)-5-((4-(4-(trifluoromethyl)piperidin-1-yl)pheny 1)amino)benzo[d]oxazol-2(3H)-one (25 mg, 22%) as a grey solid. ¹H NMR (400 MHz, DMSO) δ 7.85 (s, 1H), 7.12 (d, J=8.6 Hz, 1H), 7.02-6.94 (m, 2H), 6.94-6.86 (m, 2H), 6.83 (d, J=2.2 Hz, 1H), 6.63 (dd, J=8.6, 2.3 Hz, 1H), 3.83 (t, J=6.2 Hz, 2H), 3.62 (d, J=12.0 Hz, 2H), 2.68-2.57 (m, 2H), 2.55-2.52 (m, 2H), 2.47-2.43 (m, 1H), 2.17 (s, 6H), 1.93-1.83 (m, 2H), 1.64-1.50 (m, 2H). Mass (m/z): 449.4 [M+H]⁺.

### Compound 430

### N-(4-(2,6-dimethylmorpholino)phenyl)-2-methylisoindolin-5-amine

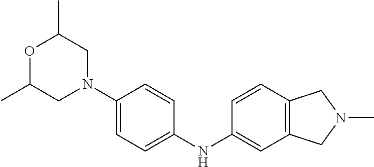

To a solution of 4-(2,6-dimethylmorpholino)aniline (100 mg, 0.48 mmol) and 5-bromo-2-methylisoindoline (113 mg, 0.53 mmol) in 1,4-dioxane (2 mL) was added Pd₂(dba)₃ (44 mg, 0.05 mmol), Xphos (46 mg, 0.10 mmol) and Cs₂CO₃ (316 mg, 0.97 mmol) under nitrogen atmosphere. The mixture was stirred at 100° C. for 12 h and the mixture was purified by flash chromatography to give N-(4-(2,6-dimethylmorpholino)phenyl)-2-methylisoindolin-5-amine (32 mg, 19%) as a light blue solid. ¹H NMR (400 MHz, DMSO) δ 8.04 (s, 1H), 7.26-7.09 (m, 1H), 7.09-6.98 (m, 2H), 6.98-6.75 (m, 3H), 4.65-4.61 (m, 2H), 4.35-4.26 (m, 2H), 3.74-3.69 (m, 2H), 2.98-2.93 (m, 3H), 2.24-2.20 (m, 2H), 1.15 (d, J=6.2 Hz, 6H). LC-MS (m/z): 338.2 [M+H]⁺.

### Compound 431

### N-(4-(2,6-dimethylmorpholino)phenyl)isoindolin-5-amine

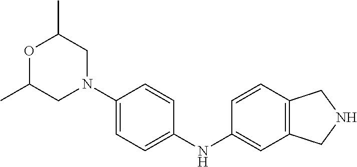 [SMILES: CC1CN(c2ccc(Nc3ccc4c(c3)CNC4)cc2)CC(C)O1]

To a solution of 5-bromoisoindoline (105 mg, 0.53 mmol) and 4-(2,6-dimethylmorpholino)aniline (100 mg, 0.48 mmol) in 1,4-dioxane (2 mL) was added Brettphos Pd G3 (44 mg, 0.04 mmol), Brettphos (52 mg, 0.10 mmol) and Cs₂CO₃ (316 mg, 0.97 mmol) under nitrogen atmosphere. The mixture was stirred at 100° C. for 12 h, the mixture was purified by prep TLC then purified by flash chromatography to give N-(4-(2,6-dimethylmorpholino)phenyl)isoindolin-5-amine (86 mg, 54%) as a light yellow solid. Mass (m/z): 324.4 [M+H]⁺. ¹H NMR (400 MHz, DMSO-d₆) δ 7.70 (s, 1H), 7.08-6.99 (m, 1H), 6.98-6.90 (m, 2H), 6.88-6.81 (m, 2H), 6.81-6.70 (m, 2H), 4.43 (d, J=7.7 Hz, 1H), 4.01 (d, J=5.4 Hz, 3H), 3.71-3.59 (m, 2H), 3.44-3.35 (m, 2H), 2.19-2.09 (m, 2H), 1.10 (d, J=6.3 Hz, 6H).

### Compound 432

### 6-((4-(2,6-dimethylmorpholino)phenyl)amino)-2-methyl-1H-indazol-3(2H)-one

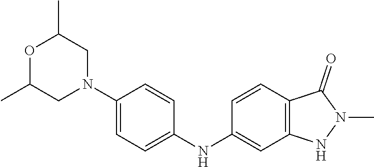

To a solution of tert-butyl 6-((4-(2,6-dimethylmorpholino)phenyl)amino)-2-methyl-3-oxo-2,3-dihydro-1H-indazole-1-carboxylate (100 mg, 0.22 mmol) in DCM (3 mL) was added 2,2,2-trifluoroacetic acid (1 mL). The mixture was stirred at r.t. for 1 h, the mixture was concentrated and purified by flash chromatography to give 6-((4-(2,6-dimethylmorpholino) phenyl)amino)-2-methyl-1H-indazol-3(2H)-one (32 mg, 41%) as a white solid. Mass (m/z): 353.4 [M+H]⁺. ¹H NMR (400 MHz, DMSO-d₆) δ 9.63 (s, 1H), 8.20 (s, 1H), 7.32 (d, J=8.6 Hz, 1H), 7.07-6.99 (m, 2H), 6.94-6.86 (m, 2H), 6.57 (dd, J=8.6, 1.9 Hz, 1H), 6.46 (d, J=1.8 Hz, 1H), 3.72-3.60 (m, 2H), 3.51-3.43 (m, 2H), 3.18 (s, 3H), 2.23-2.13 (m, 2H), 1.12 (d, J=6.2 Hz, 6H).

### Compound 433

### 5-((4-(4-(trifluoromethyl)piperidin-1-yl)phenyl)amino) benzo[d]oxazol-2(3H)-one

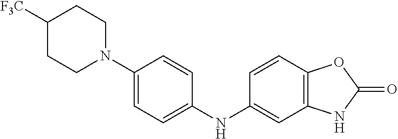 [SMILES: O=c1[nH]c2cc(Nc3ccc(N4CCC(C(F)(F)F)CC4)cc3)ccc2o1]

The title compound 433 (3.8 mg) was prepared in a total yield of 4.3% as a white solid from 4-(4-(trifluoromethyl)piperidin-1-yl)aniline (64 mg, 0.259 mmol), 5-bromobenzo[d]oxazol-2(3H)-one (50 mg, 0.236 mmol), Pd₂(dba)₃ (2 mg, 0.002 mmol), X-phos (6 mg, 0.01 mmol), Cs₂CO₃ (115 mg, 0.354 mmol) and 1,4-dioxane (5 mL) according to the procedure for compound 1. ¹H NMR (400 MHz, DMSO-d₆) δ 7.24 (s, 1H), 6.88-6.78 (m, 3H), 6.65 (d, J=8.0 Hz, 1H), 6.45 (d, J=2.4 Hz, 1H), 6.25 (dd, J=8.0, 2.0 Hz, 1H), 3.53 (d, J=12.0 Hz, 2H), 2.61-2.54 (m, 2H), 2.38 (dt, J=8.8, 4.8 Hz, 1H), 1.87 (d, J=12.0 Hz, 2H), 1.62-1.54 (m, 2H). Mass (m/z): 378.3 [M+H]⁺.

### Compound 434

### N-(4-(2,6-dimethylmorpholino)phenyl)-1-methyl-1H-benzo[d]imidazol-5-amine

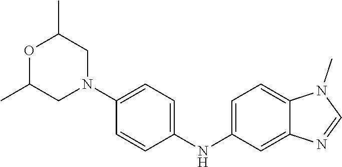 [SMILES: CC1CN(c2ccc(Nc3ccc4c(c3)ncn4C)cc2)CC(C)O1]

A mixture solution of 4-(2,6-dimethylmorpholino)aniline (0.2 g, 0.97 mmol), 5-bromo-1-methyl-1H-benzo[d]imidazole (0.22 g, 1.07 mmol), Pd₂(dba)₃ (88 mg, 0.1 mmol), Ruphos (89 mg, 0.2 mmol), Cs₂CO₃ (0.95 g, 0.3 mmol) and 1,4-dioxane (10 mL) was stirred at 100° C. under N₂ atmosphere for 12 hrs. The mixture was concentrated and the residue was purified by prep-HPLC to give 434 as a yellow solid (0.11 g, 34.0%). Mass (m/z): 336.8 [M+H]⁺. ¹H NMR (400 MHz, DMSO-d₆) δ 7.98 (s, 1H), 7.60 (s, 1H), 7.34 (d, J=8.4 Hz, 1H), 7.15 (d, J=2.0 Hz, 1H), 6.94-6.90 (m, 3H), 6.86-6.78 (m, 2H), 3.73 (s, 3H), 3.69-3.62 (m, 2H), 3.37 (d, J=10.4 Hz, 2H), 2.14 (dd, J=11.6, 10.4 Hz, 2H), 1.10 (d, J=6.0 Hz, 6H).

### Compound 435

### N-(4-(2,6-dimethylmorpholino)phenyl)-1-methyl-1H-benzo[d]imidazol-5-amine

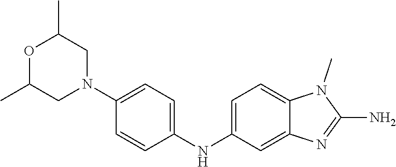 [SMILES: CC1CN(c2ccc(Nc3ccc4c(c3)nc(N)n4C)cc2)CC(C)O1]

A mixture solution of tert-butyl (5-bromo-1-methyl-1H-benzo[d]imidazol-2-yl)(tert-butoxycarbonyl)carbamate (0.26 g, 0.61 mmol), 4-(2,6-dimethylmorpholino)aniline (0.25 g, 1.22 mmol), Pd₂(dba)₃ (56 mg, 0.06 mmol), Ruphos (57 mg, 0.12 mmol), Cs₂CO₃ (0.6 g, 1.83 mmol) and dioxane (10 mL) was stirred at 100° C. under N₂ atmosphere for 12 hrs. The mixture was concentrated and the residue was purified by prep-HPLC to give compound 435 as a yellow solid (18.3 mg, 8.5%). Mass (m/z): 351.8 [M+H]⁺. ¹H NMR (400 MHz, DMSO-d₆) δ 7.33 (s, 1H), 7.06-6.72 (m, 6H), 6.59 (s, 1H), 6.26 (s, 2H), 3.69 (s, 2H), 3.44 (s, 5H), 2.17 (d, J=10.4 Hz, 2H), 1.14 (s, 6H).

### Compound 436

### 5-((4-(2,6-dimethylmorpholino)phenyl)amino)-3-methyl-1-(2-(methylamino)ethyl)-1H-benzo[d]imidazol-2(3H)-one

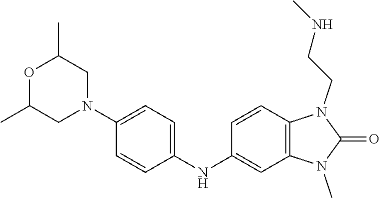 [SMILES: CNCCn1c(=O)n(C)c2cc(Nc3ccc(N4CC(C)OC(C)C4)cc3)ccc21]

To a solution of tert-butyl (2-(5-((4-(2,6-dimethylmorpholino) phenyl) amino)-3-methyl-2-oxo-2,3-dihydro-1H-benzo[d]imidazol-1-yl)ethyl) (methyl)carbamate (100 mg, 0.19 mmol) in DCM (3 mL) was added 2,2,2-trifluoroacetic acid (1 mL). The mixture was stirred at r.t. for 1 h, the mixture was concentrated and purified by prep HPLC to give 5-((4-(2,6-dimethylmorpholino)phenyl)amino)-3-methyl-1-(2-(methylamin o)ethyl)-1H-benzo[d]imidazol-2(3H)-one (23 mg, 28%) as a purple solid. Mass (m/z): 410.4 [M+H]⁺. ¹H NMR (400 MHz, DMSO-d₆) δ 7.61 (s, 1H), 7.01 (d, J=8.3 Hz, 1H), 6.98-6.91 (m, 2H), 6.89-6.81 (m, 2H), 6.73 (d, J=2.1 Hz, 1H), 6.66 (dd, J=8.3, 2.1 Hz, 1H), 3.84 (t, J=6.4 Hz, 2H), 3.75-3.63 (m, 2H), 3.45-3.37 (m, 2H), 3.24 (s, 3H), 2.75 (t, J=6.5 Hz, 2H), 2.29 (s, 3H), 2.22-2.12 (m, 2H), 1.14 (d, J=6.2 Hz, 6H).

### Compound 437

### N-(4-(2,6-dimethylmorpholino)-2-methylphenyl)-1-methyl-1H-indazol-5-amine

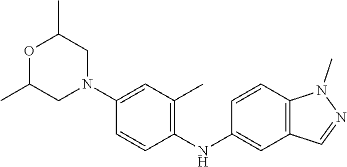 [SMILES: Cc1cc(N2CC(C)OC(C)C2)ccc1Nc1ccc2c(cnn2C)c1]

To a solution of 5-bromo-1-methyl-1H-indazole (105 mg, 0.5 mmol) and 4-(2,6-dimethylmorpholino)-2-methylaniline (100 mg, 0.45 mmol) in 1,4-dioxane (2 mL) was added Pd₂(dba)₃ (41 mg, 0.04 mmol), Xphos (43 mg, 0.09 mmol) and Cs₂CO₃ (296 mg, 0.91 mmol) under nitrogen atmosphere. The mixture was stirred at 100° C. for 12 h, the mixture was purified by flash chromatography to give N-(4-(2,6-dimethylmorpholino)-2-methylphenyl)-1-methyl-1H-indazol-5-amine (15 mg, 9%) as a light brown solid. Mass (m/z): 351.4 [M+H]⁺. ¹H NMR (400 MHz, DMSO-d₆) δ 7.73 (d, J=1.0 Hz, 1H), 7.44 (d, J=8.9 Hz, 1H), 7.05-6.98 (m, 2H), 6.97 (d, J=8.7 Hz, 1H), 6.84 (d, J=2.8 Hz, 1H), 6.77-6.70 (m, 2H), 3.96 (s, 3H), 3.75-3.63 (m, 2H), 3.52-3.44 (m, 2H), 2.25-2.16 (m, 2H), 2.14 (s, 3H), 1.15 (d, J=6.2 Hz, 6H).

### Compound 438

### 5-((4-(2,6-dimethylmorpholino)-2-methylphenyl)amino)-1,3,3-trimethylindolin-2-one

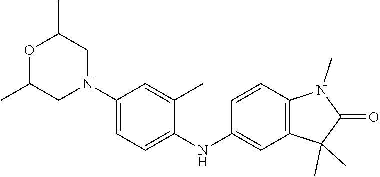 [SMILES: Cc1cc(N2CC(C)OC(C)C2)ccc1Nc1ccc2c(c1)C(C)(C)C(=O)N2C]

To a solution of 5-bromo-1,3,3-trimethylindolin-2-one (126 mg, 0.5 mmol) and 4-(2,6-dimethylmorpholino)-2-methylaniline (100 mg, 0.45 mmol) in 1,4-dioxane (2 mL) was added Pd₂(dba)₃ (41 mg, 0.04 mmol), Xphos (43 mg, 0.09 mmol) and Cs₂CO₃ (296 mg, 0.91 mmol) under nitrogen atmosphere. The mixture was stirred at 100° C. for 12 h, the mixture was purified by flash chromatography to give 5-((4-(2,6-dimethylmorpholino)-2-methylphenyl)amino)-1,3,3-trimethylindolin-2-one (48 mg, 26.8%) as a light pink solid. Mass (m/z): 395.4 [M+H]⁺. ¹H NMR (400 MHz, DMSO-d₆) δ 6.99-6.92 (m, 2H), 6.84-6.75 (m, 3H), 6.71 (dd, J=8.6, 2.9 Hz, 1H), 6.56 (dd, J=8.3, 2.2 Hz, 1H), 3.74-3.62 (m, 2H), 3.52-3.43 (m, 2H), 3.07 (s, 3H), 2.24-2.15 (m, 2H), 2.13 (s, 3H), 1.21 (s, 6H), 1.14 (d, J=6.2 Hz, 6H).

### Compound 439

### N-(4-(2,6-dimethylmorpholino)-2-methylphenyl)-2-methylisoindolin-5-amine

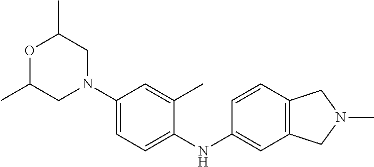

To a solution of 4-(2,6-dimethylmorpholino)-2-methylaniline (100 mg, 0.45 mmol) and 5-bromo-2-methylisoindoline (86 mg, 0.40 mmol) in 1,4-dioxane (2 mL) was added Pd₂(dba)₃ (41 mg, 0.05 mmol), Xphos (43 mg, 0.09 mmol) and Cs₂CO₃ (296 mg, 0.91 mmol) under nitrogen atmosphere. The mixture was stirred at 100° C. for 12 h, the mixture was purified by flash chromatography to give N-(4-(2,6-dimethylmorpholino)-2-methylphenyl)-2-methylisoindolin-5-amine (15 mg, 9%) as a grey solid. ¹H NMR (400 MHz, DMSO-d₆) δ 7.50 (s, 1H), 7.09 (d, J=8.3 Hz, 1H), 6.98 (d, J=8.5 Hz, 1H), 6.90 (s, 1H), 6.81 (s, 1H), 6.64-6.58 (m, 1H), 6.53 (s, 1H), 4.63-4.55 (m, 2H), 4.31-4.21 (m, 2H), 3.73-3.66 (m, 2H), 3.54-3.46 (m, 2H), 2.93 (d, J=4.8 Hz, 3H), 2.28-2.24 (m, 2H), 2.10 (s, 3H), 1.14 (d, J=6.2 Hz, 6H).

### Compound 440

### N-(4-(2,6-dimethylmorpholino)-2-methylphenyl)isoindolin-5-amine

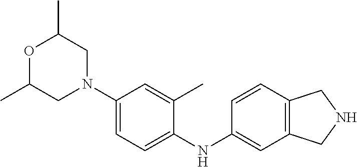 [SMILES: Cc1cc(N2CC(C)OC(C)C2)ccc1Nc1ccc2c(c1)CNC2]

To a solution of 5-bromoisoindoline (99 mg, 0.50 mmol) and 4-(2,6-dimethylmorpholino)-2-methylaniline (100 mg, 0.45 mmol) in 1,4-dioxane (2 mL) was added Brettphos Pd G3 (41 mg, 0.05 mmol), Brettphos (49 mg, 0.09 mmol) and Cs₂CO₃ (296 mg, 0.91 mmol) under a nitrogen atmosphere. The mixture was stirred at 100° C. for 12 h, the mixture was purified by prep TLC then purified by flash chromatography to give N-(4-(2,6-dimethylmorpholino)-2-methylphenyl)isoindolin-5-amine (71 mg, 48%) as a grey solid. Mass (m/z): 338.4 [M+H]⁺. ¹H NMR (400 MHz, DMSO) δ 9.64 (s, 1H), 7.45 (s, 1H), 7.08 (d, J=8.3 Hz, 1H), 6.97 (d, J=8.5 Hz, 1H), 6.86 (s, 1H), 6.77 (s, 1H), 6.61 (d, J=8.3 Hz, 1H), 6.51 (s, 1H), 4.31 (d, J=5.6 Hz, 4H), 3.70-3.66 (m, 2H), 3.50 (d, J=11.6 Hz, 2H), 2.24-2.19 (m, 2H), 2.08 (d, J=14.6 Hz, 3H), 1.13 (d, J=6.2 Hz, 6H).

### Compound 441

### 5-((4-(2,6-dimethylmorpholino)phenyl)amino)benzo[d]oxazol-2(3H)-one

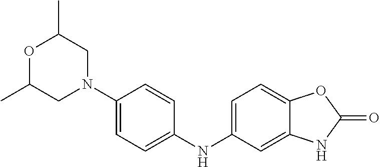 [SMILES: CC1CN(c2ccc(Nc3ccc4oc(=O)[nH]c4c3)cc2)CC(C)O1]

To a solution of 5-bromobenzo[d]oxazol-2(3H)-one (200 mg, 0.93 mmol) and 4-(2,6-dimethylmorpholino)aniline (193 mg, 0.93 mmol) in t-BuOH (5 mL) was added Pd₂(dba)₃ (42 mg, 0.05 mmol), Brettphos (50.1 mg, 0.09 mmol) and Cs₂CO₃ (609 mg, 1.87 mmol) under nitrogen atmosphere. The mixture was stirred at 100° C. for overnight. The reaction mixture was diluted with water (20 mL) and extracted with ethyl acetate (3×50 mL). The combined organic phases were dried and concentrated. The residue was purified by Prep-TLC (Petroleum ether:Ethyl acetate=1:5) and further purified by Prep-HPLC with the following conditions: Column: Spherical C18 40-60 um, 40 g; Mobile phase A: water (0.5% NH₄HCO₃); Mobile phase B: acetonitrile or ACN; Flow rate: 50 mL/min; Gradient: 20% B-65% B in 20 min; Detector: 254 nm. The fractions containing desired product were collected at 60% B and concentrated under reduced pressure to give the titled compound 441 (34.2 mg, 10.8%) as a white solid. LC-MS (m/z) 340.2 [M+H]⁺. ¹H NMR (400 MHz, DMSO-d₆) δ 11.31 (s, 1H), 7.74 (s, 1H), 7.06 (d, J=8.5 Hz, 1H), 7.01-6.93 (m, 2H), 6.92-6.84 (m, 2H), 6.63-6.53 (m, 2H), 3.69 (dqd, J=12.5, 6.1, 2.2 Hz, 2H), 3.44 (dt, J=10.7, 2.0 Hz, 2H), 2.19 (dd, J=11.8, 10.2 Hz, 2H), 1.14 (d, J=6.2 Hz, 6H).

### Compound 442

### 6-((4-(2,6-dimethylmorpholino)phenyl)amino)-1-methyl-1H-indole-3-carboxamide

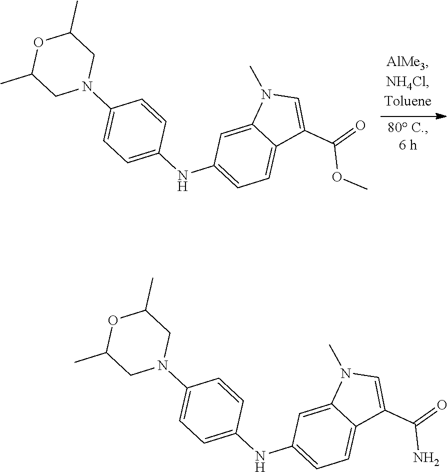

To a suspension of NH₄Cl in toluene at 0° C. was treated with AlMe₃, and the mixture was stirred at room temperature for 10 min. Methyl 6-((4-(2,6-dimethylmorpholino)phenyl)amino)-1-methyl-1H-indole-3-carb oxylate (85 mg, 0.22 mmol) in toluene (5 mL) was added to the above mixture. The mixture was heated to 80° C. and stirred for 6 h. The reaction mixture was cooled and quenched with water (10 mL) and extracted with ethyl acetate (3×20 mL). The combined organic phases were dried and concentrated. The residue was purified by Prep-TLC (Petroleum ether:Ethyl acetate=1:10) to give the titled compound 442 (44.4 mg, 54.3%) as a white solid. LC-MS (m/z) 379.2 [M+H]⁺. ¹H NMR (400 MHz, DMSO-d₆) δ 8.04-7.6 (m, 3H), 7.75-6.52 (m, 8H), 3.74 (s, 3H), 3.69 (dqd, J=12.5, 6.1, 2.2 Hz, 2H), 3.44 (dt, J=10.7, 2.0 Hz, 2H), 2.19 (dd, J=11.8, 10.2 Hz, 2H), 1.14 (d, J=6.2 Hz, 6H).

### Compound 443

### N-(4-(2,6-dimethylmorpholino)-2-methylphenyl)-1H-indazol-5-amine

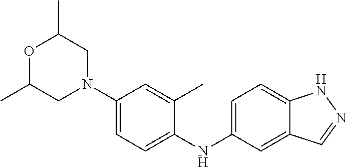 [SMILES: Cc1cc(N2CC(C)OC(C)C2)ccc1Nc1ccc2[nH]ncc2c1]

A mixture of tert-butyl 5-((4-(2,6-dimethylmorpholino)-2-methylphenyl) amino)-1H-indazole-1-carboxylate (110 mg, 0.25 mmol) in HCl (4M in dioxane, 5 mL) was stirred at room temperature for 2 h. The reaction mixture was concentrated under vacuum. The residue was diluted with a saturated solution of NaHCO₃ (30 mL) and extracted with dichloromethane (3×50 mL). The combined organic phases were dried and concentrated. The residue was purified by Prep-HPLC with the following conditions: Column: Spherical C18 40-60 um, 40 g; Mobile phase A: water (0.5% NH₄HCO₃); Mobile phase B: acetonitrile or ACN; Flow rate: 45 mL/min; Gradient: 20% B-65% B in 20 min; Detector: 254 nm. The fractions containing desired product were collected at 45% B and concentrated under reduced pressure to give the titled compound 443 (11.7 mg, 13.8%) as a white solid. LC-MS (m/z) 337.2 [M+H]⁺. ¹H NMR (400 MHz, DMSO-d₆) δ 11.31 (s, 1H), 7.74 (s, 1H), 7.06 (d, J=8.5 Hz, 1H), 7.01-6.93 (m, 2H), 6.92-6.84 (m, 2H), 6.63-6.53 (m, 2H), 3.83-3.61 (m, 2H), 3.44 (dt, J=10.7, 2.0 Hz, 2H), 3.33-3.29 (m, 2H), 2.19 (s, 3H), 1.14 (d, J=6.2 Hz, 6H).

### Compound 444

### 5-((4-(2,6-dimethylmorpholino)-2-methylphenyl)amino)-1-methyl-1H-indole-3-carboxamide

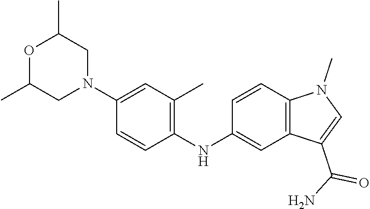 [SMILES: Cc1cc(N2CC(C)OC(C)C2)ccc1Nc1ccc2c(c1)c(C(N)=O)cn2C]

To a solution of 5-((4-(2,6-dimethylmorpholino)-2-methylphenyl) amino)-1-methyl-1H-indole-3-carboxylic acid (50 mg, 0.127 mmol) in DMF (5 mL) was added HATU (58 mg, 0.152 mmol), NH₄Cl (8.1 mg, 0.152 mmol) and TEA (19.2 mg, 0.191 mmol). The mixture was stirred at 80° C. for 2 hours. Then the mixture was diluted with water and extracted by EA (25 mL×3). The combined organic layers were washed with brine (15 mL×3), dried over Na₂SO₄ and concentrated to give the crude product, which was purified by pre-HPLC to give the desired product as pink solid (3 mg, 6.1%). ¹H NMR (400 MHz, DMSO-d₆) δ 7.83 (s, 1H), 7.54 (d, J=2.0 Hz, 1H), 7.26 (d, J=8.8 Hz, 1H), 6.96 (d, J=8.6 Hz, 1H), 6.89-6.64 (m, 4H), 3.83-3.60 (m, 5H), 3.46 (d, J=10.6 Hz, 2H), 2.25-2.08 (m, 4H), 1.14 (d, J=6.2 Hz, 6H). Mass (m/z): 393.2 [M+H]⁺.

### Compound 445

### N-(4-(2,6-dimethylmorpholino)-2-methylphenyl)-2-methyl-2H-indazol-5-amine

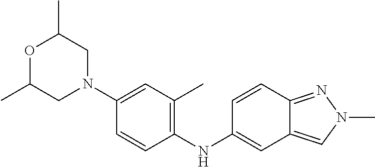

To a solution of 5-bromo-2-methyl-2H-indazole (105 mg, 0.5 mmol) and 4-(2,6-dimethylmorpholino)-2-methylaniline (100 mg, 0.45 mmol) in 1,4-dioxane (2 mL) was added Pd₂(dba)₃ (41 mg, 0.04 mmol), Xphos (43 mg, 0.09 mmol) and Cs₂CO₃ (296 mg, 0.91 mmol) under nitrogen atmosphere. The mixture was stirred at 100° C. for 12 h, the mixture was purified by flash chromatography to give N-(4-(2,6-dimethylmorpholino)-2-methylphenyl)-2-methyl-2H-indazol-5-amine (24.5 mg, 15%) as a grey solid. Mass (m/z): 351.4 [M+H]⁺. ¹H NMR (400 MHz, DMSO-d₆) δ 7.90 (d, J=0.9 Hz, 1H), 7.41 (d, J=9.1 Hz, 1H), 7.02-6.96 (m, 2H), 6.93 (dd, J=9.2, 2.2 Hz, 1H), 6.84 (d, J=2.8 Hz, 1H), 6.74 (dd, J=8.6, 2.9 Hz, 1H), 6.50 (d, J=2.0 Hz, 1H), 4.04 (s, 3H), 3.75-3.63 (m, 2H), 3.53-3.45 (m, 2H), 2.26-2.16 (m, 2H), 2.14 (s, 3H), 1.15 (d, J=6.2 Hz, 6H).

### Compound 446

### 5-((4-(2,6-dimethylmorpholino)-2-methylphenyl)amino)-3-methylbenzo[d]oxazol-2(3H)-one

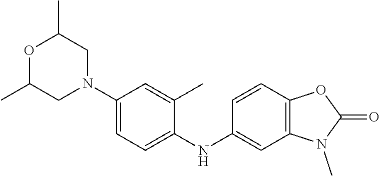 [SMILES: Cc1cc(N2CC(C)OC(C)C2)ccc1Nc1ccc2oc(=O)n(C)c2c1]

To a solution of 4-(2,6-dimethylmorpholino)-2-methylaniline (100 mg, 0.45 mmol) and 5-bromo-3-methylbenzo[d]oxazol-2(3H)-one (113 mg, 0.5 mmol) in 1,4-dioxane (2 mL) was added Pd₂(dba)₃ (41 mg, 0.05 mmol), Xphos (43 mg, 0.09 mmol) and Cs₂CO₃ (296 mg, 0.91 mmol) under nitrogen atmosphere. The mixture was stirred at 100° C. for 12 h, the mixture was purified by prep TLC to give 5-((4-(2,6-dimethylmorpholino)-2-methylphenyl)amino)-3-methylbenzo[d]oxazol-2(3H)-one (69 mg, 41%) as a light blue solid. Mass (m/z): 368.4 [M+H]⁺. ¹H NMR (400 MHz, DMSO-d₆) δ 7.26 (s, 1H), 7.03 (dd, J=16.8, 8.6 Hz, 2H), 6.85 (d, J=2.8 Hz, 1H), 6.75 (dd, J=8.6, 2.9 Hz, 1H), 6.44 (d, J=2.2 Hz, 1H), 6.35 (dd, J=8.6, 2.3 Hz, 1H), 3.74-3.62 (m, 2H), 3.55-3.47 (m, 2H), 3.22 (s, 3H), 2.26-2.16 (m, 2H), 2.14 (s, 3H), 1.15 (d, J=6.2 Hz, 6H).

### Compound 447

### N-(4-(2,6-dimethylmorpholino)-2-methylphenyl)-1-methyl-1H-benzo[d]imidazol-5-amine

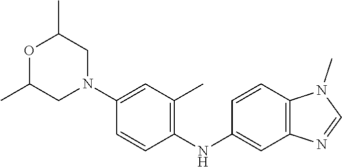 [SMILES: Cc1cc(N2CC(C)OC(C)C2)ccc1Nc1ccc2c(c1)ncn2C]

To a solution of 5-bromo-1-methyl-1H-benzo[d]imidazole (105 mg, 0.5 mmol) and 4-(2,6-dimethylmorpholino)-2-methylaniline (100 mg, 0.45 mmol) in 1,4-dioxane (2 mL) was added Pd₂(dba)₃ (41 mg, 0.04 mmol), Xphos (43 mg, 0.09 mmol) and Cs₂CO₃ (296 mg, 0.91 mmol) under nitrogen atmosphere. The mixture was stirred at 100° C. for 12 h, the mixture was purified by flash chromatography to give N-(4-(2,6-dimethylmorpholino)-2-methylphenyl)-1-methyl-1H-benzo[d]imidazol-5-a mine (45 mg, 28%) as a pink solid. Mass (m/z): 351.4 [M+H]⁺. ¹H NMR (400 MHz, DMSO) δ 7.96 (s, 1H), 7.32 (d, J=9.1 Hz, 1H), 6.98 (d, J=8.6 Hz, 1H), 6.94 (s, 1H), 6.86-6.78 (m, 3H), 6.72 (dd, J=8.7, 2.9 Hz, 1H), 3.75 (s, 3H), 3.73-3.63 (m, 2H), 3.52-3.44 (m, 2H), 2.25-2.16 (m, 2H), 2.15 (s, 3H), 1.15 (d, J=6.2 Hz, 6H).

### Compound 448

### 7-({2-chloro-4-[4-(trifluoromethyl)piperidin-1-yl]phenyl}amino)-2,4-dihydro-1,4-benzoxazin-3-one

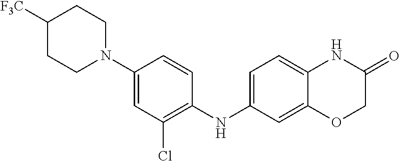 [SMILES: O=C1COc2cc(Nc3ccc(N4CCC(C(F)(F)F)CC4)cc3Cl)ccc2N1]

The titled compound 448 (6.8 mg, 4.38%) as a light-yellow solid was prepared according to the procedure outlined for compound 1. ¹H NMR (400 MHz, DMSO-d₆) δ 10.45 (s, 1H), 7.34-7.27 (m, 1H), 7.26-7.00 (m, 2H), 6.91 (s, 1H), 6.69 (s, 1H), 6.41 (s, 2H), 4.47 (s, 2H), 3.80-3.66 (m, 2H), 3.51 (br, 1H), 2.76-2.62 (m, 2H), 1.86 (s, 2H), 1.56 (s, 2H). Mass (m/z): 425.9 [M+H]⁺.

### Compound 449

### 6-((4-(2,6-dimethylmorpholino)phenyl)amino)-1,3,3-trimethylindolin-2-one

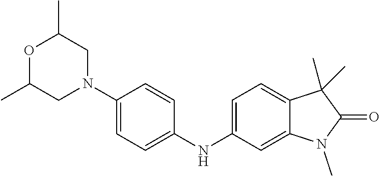 [SMILES: CC1CN(c2ccc(Nc3ccc4c(c3)N(C)C(=O)C4(C)C)cc2)CC(C)O1]

The titled compound 449 (3.8 mg, 2.06%) as a light-brown solid was prepared according to the procedure outlined for compound 1. ¹H NMR (400 MHz, DMSO-d₆) δ 7.86 (s, 1H), 7.06 (d, J=8.0 Hz, 1H), 7.01 (d, J=8.8 Hz, 2H), 6.88 (d, J=9.2 Hz, 2H), 6.56-6.49 (m, 2H), 3.72-3.64 (m, 2H), 3.45 (d, J=10.4 Hz, 2H), 3.05 (s, 3H), 2.22-2.15 (m, 2H), 1.22 (s, 6H), 1.14 (d, J=6.4 Hz, 6H). Mass (m/z): 380.0 [M+H]⁺.

### Compound 450

### 7-((4-(4-(trifluoromethyl)piperidin-1-yl)phenyl)amino)-2H-pyrido[3,2-b][1,4]oxazin-3(4H)-one

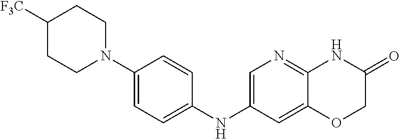 [SMILES: O=C1COc2cc(Nc3ccc(N4CCC(C(F)(F)F)CC4)cc3)cnc2N1]

The titled compound 450 (3.5 mg, 2.15%) as a light-purple solid was prepared according to the procedure outlined for compound 1. ¹H NMR (400 MHz, DMSO-d₆) δ 7.85 (s, 1H), 7.59 (d, J=2.4 Hz, 1H), 7.00-6.87 (m, 5H), 4.57 (s, 2H), 3.62 (d, J=12.4 Hz, 2H), 2.62 (t, J=11.2 Hz, 2H), 2.45-2.39 (m, 1H), 1.88 (d, J=12.8 Hz, 2H), 1.61-1.53 (m, 2H). Mass (m/z): 392.9 [M+H]⁺.

### Compound 451

### 7-((2-chloro-4-(4-(trifluoromethyl)piperidin-1-yl)phenyl)amino)-4-methyl-2H-benzo[b][1,4]oxazin-3(4H)-one

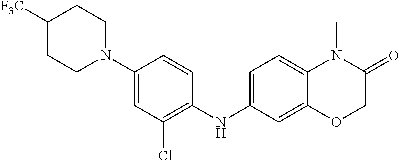 [SMILES: CN1C(=O)COc2cc(Nc3ccc(N4CCC(C(F)(F)F)CC4)cc3Cl)ccc21]

The titled compound 451 (19.4 mg, 12.1%) as a light-purple solid was prepared according to the procedure outlined for compound 1. ¹H NMR (400 MHz, DMSO-d₆) δ 7.45 (s, 1H), 7.14 (d, J=8.8 Hz, 1H), 7.04 (d, J=2.8 Hz, 1H), 6.97-6.89 (m, 2H), 6.48 (dd, J=8.8, 2.4 Hz, 1H), 6.42 (d, J=2.4 Hz, 1H), 4.56 (s, 2H), 3.73 (d, J=12.0 Hz, 2H), 3.22 (s, 3H), 2.69 (dd, J=12.4, 10.4 Hz, 2H), 1.88 (d, J=12.4 Hz, 2H), 1.54 (dt, J=12.4, 8.8 Hz, 2H). Mass (m/z): 439.9 [M+H]⁺.

### Compound 452

### 4-methyl-7-((4-methyl-6-(4-(trifluoromethyl)piperidin-1-yl)pyridin-3-yl) amino)-2H-benzo[b][1,4]oxazin-3(4H)-one

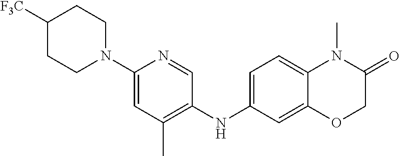 [SMILES: Cc1cc(N2CCC(C(F)(F)F)CC2)ncc1Nc1ccc2c(c1)OCC(=O)N2C]

The titled compound 452 (54.9 mg, 32.20%) as a white solid was prepared according to the procedure outlined for compound 1. ¹H NMR (400 MHz, DMSO-d₆) δ 7.87 (s, 1H), 7.29 (s, 1H), 6.91 (d, J=8.8 Hz, 1H), 6.82 (s, 1H), 6.24 (dd, J=8.8, 2.4 Hz, 1H), 6.18 (d, J=2.4 Hz, 1H), 4.54 (s, 2H), 4.35 (d, J=13.2 Hz, 2H), 3.20 (s, 3H), 2.80 (t, J=12.0 Hz, 2H), 2.66-2.54 (m, 1H), 2.09 (s, 3H), 1.85 (d, J=11.2 Hz, 2H), 1.46-1.21 (m, 2H). Mass (m/z): 421.0 [M+H]⁺.

### Compound 453

### 7-((4-methyl-6-(4-(trifluoromethyl)piperidin-1-yl)pyridin-3-yl)amino)-2H-benzo[b][1,4]oxazin-3(4H)-one

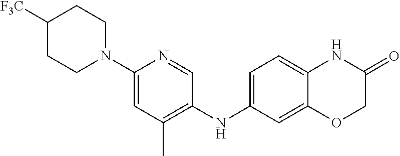 [SMILES: Cc1cc(N2CCC(C(F)(F)F)CC2)ncc1Nc1ccc2c(c1)OCC(=O)N2]

The titled compound 453 (23.5 mg, 14.23%) as a white solid was prepared according to the procedure outlined for compound 1. ¹H NMR (400 MHz, DMSO-d₆) δ 10.38 (s, 1H), 7.85 (s, 1H), 7.16 (s, 1H), 6.80 (s, 1H), 6.65 (d, J=8.0 Hz, 1H), 6.16 (d, J=8.4 Hz, 2H), 4.45 (s, 2H), 4.34 (d, J=12.4 Hz, 2H), 2.79 (t, J=12.4 Hz, 2H), 2.53 (s, 1H), 2.08 (s, 3H), 1.85 (d, J=11.6 Hz, 2H), 1.43 (d, J=11.6 Hz, 2H). Mass (m/z): 407.0 [M+H]⁺.

### Compound 454

### 7-((3-fluoro-2-methyl-4-(4-(trifluoromethyl)piperidin-1-yl)phenyl) amino)-2H-benzo[b][1,4]oxazin-3(4H)-one

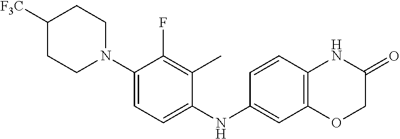 [SMILES: Cc1c(Nc2ccc3c(c2)OCC(=O)N3)ccc(N2CCC(C(F)(F)F)CC2)c1F]

The titled compound 454 (21.4 mg, 13.95%) as a white solid was prepared according to the procedure outlined for compound 1. ¹H NMR (400 MHz, DMSO-d₆) δ 10.46 (s, 1H), 7.31 (s, 1H), 6.91-6.78 (m, 2H), 6.70 (d, J=8.0 Hz, 1H), 6.43-6.36 (m, 2H), 4.48 (s, 2H), 3.31 (s, 1H), 2.65 (t, J=11.2 Hz, 2H), 2.49-2.35 (m, 2H), 2.06 (t, J=6.0 Hz, 3H), 1.89 (d, J=11.6 Hz, 2H), 1.65-1.52 (m, 2H). Mass (m/z): 424.0 [M+H]⁺.

### Compound 455

### 7-((4-(2,6-dimethylmorpholino)-3-methylphenyl)amino)-4-(2-hydroxyethyl)-2H-benzo[b][1,4]oxazin-3(4H)-one

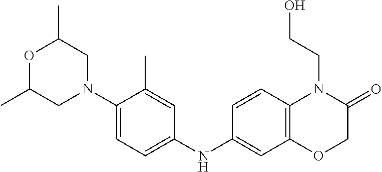 [SMILES: Cc1cc(Nc2ccc3c(c2)OCC(=O)N3CCO)ccc1N1CC(C)OC(C)C1]

The titled compound 455 (12.1 mg, 29.63%) as a light-purple solid was prepared according to the procedure outlined for compound 1. ¹H NMR (400 MHz, DMSO-d₆) δ 7.90 (s, 1H), 7.10 (d, J=8.8 Hz, 1H), 6.92 (d, J=8.4 Hz, 1H), 6.87-6.82 (m, 2H), 6.67 (dd, J=8.8, 2.4 Hz, 1H), 6.59 (d, J=2.4 Hz, 1H), 4.87 (t, J=5.6 Hz, 1H), 4.55 (s, 2H), 3.90 (t, J=6.4 Hz, 2H), 3.75-3.68 (m, 2H), 3.56 (q, J=6.4 Hz, 2H), 2.84 (d, J=10.8 Hz, 2H), 2.28 (dd, J=11.6, 10.0 Hz, 2H), 2.21 (s, 3H), 1.10 (d, J=6.4 Hz, 6H). Mass (m/z): 412.0 [M+H]⁺.

### Compound 456

### 7-((4-(2,6-dimethylmorpholino)-2-methylphenyl)amino)-4-(2-hydroxyethyl)-2H-benzo[b][1,4]oxazin-3(4H)-one

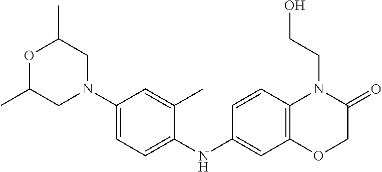 [SMILES: Cc1cc(N2CC(C)OC(C)C2)ccc1Nc1ccc2c(c1)OCC(=O)N2CCO]

The titled compound 456 (36.0 mg, 57.33%) as a light-purple solid was prepared according to the procedure outlined for compound 1. ¹H NMR (400 MHz, DMSO-d₆) δ 7.17 (s, 1H), 6.96 (dd, J=13.6, 8.8 Hz, 2H), 6.80 (d, J=2.8 Hz, 1H), 6.71 (dd, J=8.8, 2.8 Hz, 1H), 6.30 (dd, J=8.8, 2.4 Hz, 1H), 6.21 (d, J=2.4 Hz, 1H), 6.04 (s, 1H), 4.47 (s, 2H), 3.83 (t, J=6.4 Hz, 2H), 3.67-3.60 (m, 2H), 3.53-3.45 (m, 4H), 2.20-2.14 (m, 2H), 2.08 (s, 3H), 1.11 (d, J=6.4 Hz, 6H). Mass (m/z): 412.0 [M+H]⁺.

### Compound 457

### 7-((4-(2,6-dimethylmorpholino)-2-methylphenyl)amino)-4-methyl-2H-pyrido[3,2-b][1,4]oxazin-3(4H)-one

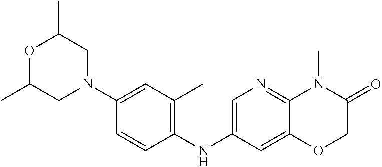 [SMILES: Cc1cc(N2CC(C)OC(C)C2)ccc1Nc1cnc2c(c1)OCC(=O)N2C]

The titled compound 457 (126 mg, 48.6%) as a yellow solid was prepared according to the procedure outlined for compound 1. ¹H NMR (400 MHz, DMSO-d₆) δ 7.47 (d, J=2.4 Hz, 1H), 7.38 (s, 1H), 6.99 (d, J=8.8 Hz, 1H), 6.85 (d, J=2.8 Hz, 1H), 6.76 (dd, J=8.8, 2.8 Hz, 1H), 6.60 (d, J=2.4 Hz, 1H), 4.66 (s, 2H), 3.69-3.65 (m, 2H), 3.51 (d, J=10.4 Hz, 2H), 3.26 (s, 3H), 2.23-2.20 (m, 2H), 2.14 (s, 3H), 1.15 (d, J=6.4 Hz, 6H). Mass (m/z): 383.0 [M+H]⁺.

### Compound 458

### N-(4-(2,6-dimethylmorpholino) phenyl)-1-methyl-1H-benzo[d][1,2,3]triazol-5-amine

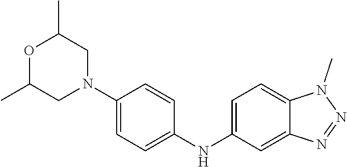 [SMILES: CC1CN(c2ccc(Nc3ccc4c(c3)nnn4C)cc2)CC(C)O1]

The titled compound 458 (61.8 mg, 34.55%) as a green solid was prepared according to the procedure outlined for compound 1. ¹H NMR (400 MHz, DMSO-d₆) δ 7.99 (s, 1H), 7.66-7.64 (m, 1H), 7.28 (s, 2H), 7.20-7.18 (m, 1H), 7.08-7.05 (d, J=12 Hz, 2H), 6.94-6.91 (m, 2H), 4.22 (s, 3H), 3.70-3.68 (m, 2H), 3.48-3.46 (m, 2H), 2.24-2.21 (m, 2H), 1.16-1.14 (m, 6H). Mass (m/z): 338.0 [M+H]⁺.

### Compound 459

### 7-((5-fluoro-6-(4-(trifluoromethyl)piperidin-1-yl)pyridin-3-yl)amino)-2H-benzo[b][1,4]oxazin-3(4H)-one

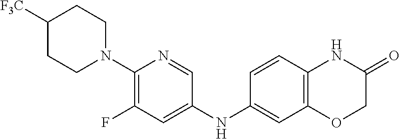 [SMILES: O=C1COc2cc(Nc3cnc(N4CCC(C(F)(F)F)CC4)c(F)c3)ccc2N1]

The titled compound 459 (70.6 mg, 45.31%) as a yellow solid was prepared according to the procedure outlined for compound 1. ¹H NMR (400 MHz, DMSO-d₆) δ 10.53 (s, 1H), 8.06 (s, 1H), 7.81 (d, J=1.6 Hz, 1H), 7.23 (dd, J=14.4, 2.4 Hz, 1H), 6.77 (d, J=8.4 Hz, 1H), 6.62 (dd, J=8.4, 2.4 Hz, 1H), 6.57 (d, J=2.4 Hz, 1H), 4.51 (s, 2H), 3.76 (d, J=12.8 Hz, 2H), 3.34-3.33 (m, 1H), 2.80 (t, J=11.6 Hz, 2H), 1.87 (d, J=11.6 Hz, 2H), 1.56 (dd, J=12.8, 3.6 Hz, 2H). Mass (m/z): 411.0 [M+H]⁺.

### Compound 460

### 7-((2-methyl-4-(4-(trifluoromethyl)piperidin-1-yl)phenyl)amino)-2H-pyrido[3,2-b][1,4]oxazin-3(4H)-one

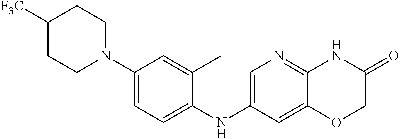 [SMILES: Cc1cc(N2CCC(C(F)(F)F)CC2)ccc1Nc1cnc2c(c1)OCC(=O)N2]

The titled compound 460 (24.7 mg, 14.51%) as a light-purple solid was prepared according to the procedure outlined for compound 1. ¹H NMR (400 MHz, DMSO-d₆) δ 7.37 (d, J=2.4 Hz, 1H), 7.26 (s, 1H), 6.98 (d, J=8.8 Hz, 1H), 6.86 (d, J=2.8 Hz, 1H), 6.77 (dd, J=8.8, 2.8 Hz, 1H), 6.60 (d, J=2.4 Hz, 1H), 4.54 (s, 2H), 3.70 (d, J=12.4 Hz, 2H), 2.65 (dd, J=12.4, 10.4 Hz, 2H), 2.48-2.39 (m, 1H), 2.13 (s, 3H), 1.88 (d, J=12.0 Hz, 2H), 1.62-1.52 (m, 2H). Mass (m/z): 407.0 [M+H]⁺.

### Compound 461

### 7-((4-(2,6-dimethylmorpholino)-3-methylphenyl)amino)-4-(2-hydroxyethyl)-2H-benzo[b][1,4]oxazin-3(4H)-one

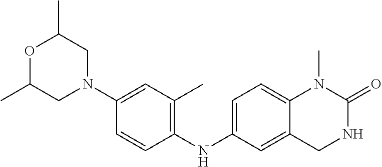 [SMILES: Cc1cc(N2CC(C)OC(C)C2)ccc1Nc1ccc2c(c1)CNC(=O)N2C]

The titled compound 461 (3.3 mg, 10.02% yield) as a grey solid was prepared according to the procedure outlined for compound 1. ¹H NMR (400 MHz, DMSO-d₆) δ 6.99-6.94 (m, 2H), 6.85-6.80 (m, 2H), 6.75-6.69 (m, 2H), 6.57 (dd, J=8.8, 2.8 Hz, 1H), 6.50 (d, J=2.4 Hz, 1H), 4.14 (s, 2H), 3.72-3.65 (m, 2H), 3.48 (d, J=10.4 Hz, 2H), 3.09 (s, 3H), 2.22-2.16 (m, 2H), 2.12 (s, 3H), 1.14 (d, J=6.4 Hz, 6H). Mass (m/z): 412.0 [M+H]⁺.

### Compound 462

### 7-((4-(2,6-dimethylmorpholino)-2-methylphenyl)amino)-2H-pyrido[3,2-b][1,4]oxazin-3(4H)-one

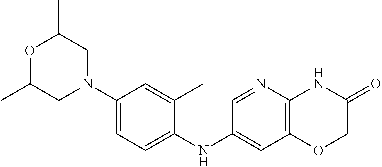 [SMILES: Cc1cc(N2CC(C)OC(C)C2)ccc1Nc1cnc2c(c1)OCC(=O)N2]

The titled compound 462 (26.0 mg, 15.33%) as a light purple solid was prepared according to the procedure outlined for compound 1. ¹H NMR (400 MHz, DMSO-d₆) δ 7.37 (d, J=2.4 Hz, 1H), 7.26 (s, 1H), 6.98 (d, J=8.8 Hz, 1H), 6.86 (d, J=2.8 Hz, 1H), 6.77 (dd, J=8.8, 2.8 Hz, 1H), 6.60 (d, J=2.4 Hz, 1H), 4.54 (s, 2H), 3.70 (d, J=12.4 Hz, 2H), 2.65 (dd, J=12.4, 10.4 Hz, 2H), 2.48-2.39 (m, 1H), 2.13 (s, 3H), 1.88 (d, J=12.0 Hz, 2H), 1.62-1.52 (m, 2H). Mass (m/z): 369.0 [M+H]⁺.

### Compound 463

### 7-{[4-(2,6-dimethylmorpholin-4-yl)phenyl]amino}-4,5-dimethyl-2H-1,4-benzoxazin-3-one

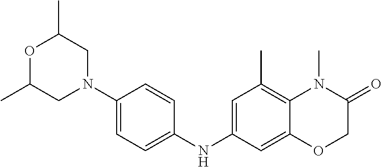 [SMILES: Cc1cc(Nc2ccc(N3CC(C)OC(C)C3)cc2)cc2c1N(C)C(=O)CO2]

The titled compound 463 (4.0 mg, 1.82%) as a light-yellow solid was prepared according to the procedure outlined for compound 1. ¹H NMR (400 MHz, DMSO-d₆) δ 7.79 (s, 1H), 6.93 (dd, J=34.4, 8.8 Hz, 4H), 6.43 (d, J=16.8 Hz, 2H), 4.40 (s, 2H), 3.76-3.61 (m, 2H), 3.45 (d, J=11.2 Hz, 2H), 3.26 (s, 3H), 2.29 (s, 3H), 2.19 (t, J=11.2 Hz, 2H), 1.14 (d, J=6.4 Hz, 6H). Mass (m/z): 382.0 [M+H]⁺.

### Compound 464

### 7-({3-methoxy-4-[4-(trifluoromethyl)piperidin-1-yl]phenyl}amino)-4-methyl-2H-pyrido[3,2-b][1,4]oxazin-3-one

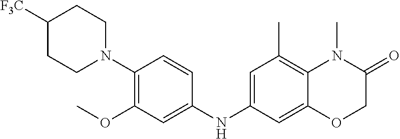 [SMILES: COc1cc(Nc2cc(C)c3c(c2)OCC(=O)N3C)ccc1N1CCC(C(F)(F)F)CC1]

The titled compound 464 (12.5 mg, 7.73%) as a light-yellow solid was prepared according to the procedure outlined for compound 1. ¹H NMR (400 MHz, DMSO-d₆) δ 8.09 (s, 1H), 7.78 (d, J=2.4 Hz, 1H), 7.02 (d, J=2.4 Hz, 1H), 6.82 (d, J=8.4 Hz, 1H), 6.65-6.56 (m, 2H), 4.70 (s, 2H), 3.75 (s, 3H), 3.29 (s, 4H), 2.59-2.51 (m, 3H), 2.43-2.32 (m, 1H), 1.86 (d, J=11.2 Hz, 2H), 1.59 (dd, J=12.4, 3.6 Hz, 2H).Mass (m/z): 437.0 [M+H]⁺.

### Compound 465

### 7-((3-methoxy-4-(4-(trifluoromethyl)piperidin-1-yl)phenyl)amino)-2H-pyrido[3,2-b][1,4]oxazin-3(4H)-one

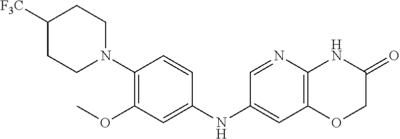 [SMILES: COc1cc(Nc2cnc3c(c2)OCC(=O)N3)ccc1N1CCC(C(F)(F)F)CC1]

The titled compound 465 (7.8 mg, 4.94%) as a light-brown solid was prepared according to the procedure outlined for compound 1. ¹H NMR (400 MHz, DMSO-d₆) δ 11.20-10.78 (m, 1H), 7.99 (s, 1H), 7.67 (d, J=2.4 Hz, 1H), 6.99 (d, J=2.4 Hz, 1H), 6.81 (d, J=8.4 Hz, 1H), 6.65-6.49 (m, 2H), 4.58 (s, 2H), 3.74 (s, 3H), 3.30 (s, 2H), 2.54 (d, J=12.0 Hz, 2H), 2.37 (dd, J=12.4, 8.8 Hz, 1H), 1.86 (d, J=11.2 Hz, 2H), 1.59 (qd, J=12.4, 3.6 Hz, 2H). Mass (m/z): 422.9 [M+H]⁺.

### Compound 466

### 7-((2-fluoro-4-(4-(trifluoromethyl)piperidin-1-yl)phenyl)amino)-2H-benzo[b][1,4]oxazin-3(4H)-one

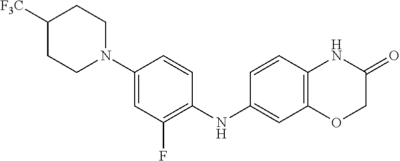 [SMILES: O=C1COc2cc(Nc3ccc(N4CCC(C(F)(F)F)CC4)cc3F)ccc2N1]

The titled compound 466 ((4.7 mg, 3.02%) as a white solid was prepared according to the procedure outlined for compound 1. ¹H NMR (400 MHz, CDCl₃) δ 7.71 (s, 1H), 7.16 (s, 1H), 6.73 (s, 1H), 6.67 (d, J=8.4 Hz, 1H), 6.61 (s, 1H), 6.56 (s, 1H), 4.58 (s, 2H), 3.67 (d, J=10.0 Hz, 2H), 2.74 (s, 2H), 2.19 (s, 1H), 2.01 (s, 2H), 1.82 (s, 2H). Mass (m/z): 410.0 [M+H]⁺.

### Compound 467

### 5-((2-methyl-4-(4-(trifluoromethyl)piperidin-1-yl)phenyl)amino) benzo[d]oxazol-2(3H)-one

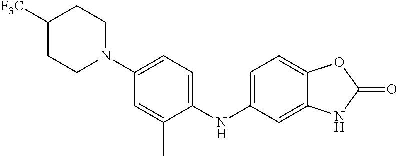 [SMILES: Cc1cc(N2CCC(C(F)(F)F)CC2)ccc1Nc1ccc2oc(=O)[nH]c2c1]

The titled compound 467 (12.0 mg, 7.9%) as a yellow solid was prepared according to the procedure outlined for compound 1. ¹H NMR (400 MHz, CD₃OD) δ 7.02 (d, J=8.8 Hz, 1H), 6.94 (d, J=8.8 Hz, 1H), 6.88 (d, J=2.4 Hz, 1H), 6.78-6.80 (m, 1H), 6.50-6.32 (m, 2H), 3.66 (d, J=12.0 Hz, 2H), 2.65-2.68 (m, 2H), 2.25-2.27 (m, 1H), 2.17 (d, J=10.0 Hz, 3H), 1.97-2.00 (m, 2H), 1.75-1.64 (m, 2H). Mass (m/z): 391.7 [M+H]⁺.

### Compound 468

### 7-((2-methyl-4-(4-(trifluoromethyl)piperidin-1-yl)phenyl)amino)-3-oxo-3,4-dihydro-2H-benzo[b][1,4]oxazine-5-carbonitrile

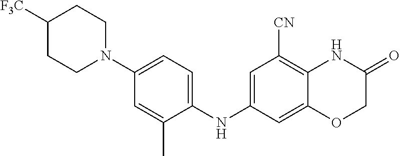 [SMILES: Cc1cc(N2CCC(C(F)(F)F)CC2)ccc1Nc1cc(C#N)c2c(c1)OCC(=O)N2]

The titled compound 468 (12.0 mg, 7.9%) as a yellow solid was prepared according to the procedure outlined for compound 1. ¹H NMR (400 MHz, DMSO-d₆) δ 10.69 (s, 1H), 7.55 (s, 1H), 6.99 (d, J=8.8 Hz, 1H), 6.89 (d, J=2.8 Hz, 1H), 6.80 (dd, J=8.8, 2.8 Hz, 1H), 6.53 (d, J=2.4 Hz, 1H), 6.42 (d, J=2.4 Hz, 1H), 4.54 (s, 2H), 3.74 (d, J=12.8 Hz, 2H), 2.67 (dd, J=12.4, 10.4 Hz, 2H), 2.45 (dd, J=12.4, 8.8 Hz, 1H), 2.10 (s, 3H), 1.91-1.85 (m, 2H), 1.60-1.50 (m, 2H). Mass (m/z): 431.0 [M+H]⁺.

### Compound 469

### 6-amino-4-methyl-7-((4-(4-(trifluoromethyl)piperidin-1-yl)phenyl) amino)-2H-benzo[b][1,4]oxazin-3(4H)-one

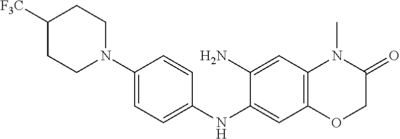 [SMILES: CN1C(=O)COc2cc(Nc3ccc(N4CCC(C(F)(F)F)CC4)cc3)c(N)cc21]

The titled compound 468 (6 mg, 3.6%) as a yellow solid was prepared according to the procedure outlined for compound 1. ¹H NMR (400 MHz, DMSO-d₆) δ 6.92-6.78 (m, 3H), 6.71 (d, J=6.8 Hz, 2H), 6.56 (s, 2H), 4.47 (s, 2H), 3.60-3.50 (m, 2H), 3.21 (s, 3H), 2.79-2.58 (m, 2H), 2.48-2.34 (m, 1H), 1.88 (d, J=12.4 Hz, 2H), 1.67-1.48 (m, 2H). Mass (m/z): 420.9 [M+H]⁺.

### Compound 470

### 7-((2-methyl-4-(piperidin-1-yl)phenyl)amino)-2H-benzo[b][1,4]oxazin-3(4H)-one

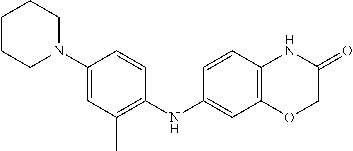 [SMILES: Cc1cc(N2CCCCC2)ccc1Nc1ccc2c(c1)OCC(=O)N2]

The titled compound 470 (32.9 mg, 20.1%) as a blue solid was prepared according to the procedure outlined for compound 1. ¹H NMR (400 MHz, DMSO-d₆) δ 12.57 (s, 1H), 10.38 (s, 1H), 7.08-6.90 (m, 2H), 6.85-6.57 (m, 3H), 6.32-6.20 (m, 1H), 4.46 (s, 2H), 2.12 (s, 3H), 1.61 (s, 4H), 1.28-1.21 (m, 4H), 0.85 (t, J=6.4 Hz, 2H). Mass (m/z): 338.3 [M+H]⁺.

### Compound 471

### 7-((6-((2R,6R)-2-ethyl-6-methylmorpholino)-2-methylpyridin-3-yl)amino)-2H-benzo[b][1,4]oxazin-3(4H)-one

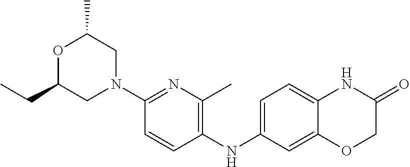 [SMILES: CC[C@@H]1CN(c2ccc(Nc3ccc4c(c3)OCC(=O)N4)c(C)n2)C[C@@H](C)O1]

The titled compound 471 (35.0 mg, 48.2%) as a light-yellow solid was prepared according to the procedure outlined for compound 1. ¹H NMR (400 MHz, DMSO-d₆) δ 7.33 (d, J=8.8 Hz, 1H), 6.68 (d, J=8.4 Hz, 1H), 6.60 (d, J=8.8 Hz, 1H), 6.28-6.18 (m, 2H), 4.47 (s, 2H), 4.09-4.01 (m, 1H), 3.87-3.75 (m, 1H), 3.70-3.61 (m, 1H), 3.53-3.44 (m, 1H), 3.39-3.33 (m, 1H), 3.14-3.04 (m, 1H), 2.28 (s, 3H), 1.81-1.69 (m, 1H), 1.63-1.52 (m, 1H), 1.25 (d, J=6.4 Hz, 3H), 0.98 (t, J=7.2 Hz, 3H). Mass (m/z): 383.2 [M+H]⁺.

### Compound 472

### 2-ethyl-4-methyl-7-((4-(tetrahydro-2H-pyran-4-yl)phenyl)amino)-2H-benzo[b][1,4]oxazin-3(4H)-one

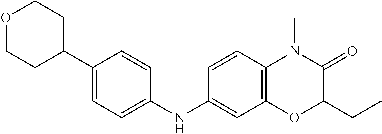 [SMILES: CCC1Oc2cc(Nc3ccc(C4CCOCC4)cc3)ccc2N(C)C1=O]

The titled compound 472 (46.0 mg, 34%) as a white solid was prepared according to the procedure outlined for compound 1. ¹H NMR (400 MHz, CD₃OD) δ 7.13 (d, J=8.0 Hz, 2H), 7.02 (d, J=8.0 Hz, 2H), 6.97 (d, J=8.4 Hz, 1H), 6.74 (dd, J=8.4, 2.0 Hz, 1H), 6.68 (d, J=2.4 Hz, 1H), 4.49-4.39 (m, 1H), 4.03 (d, J=11.2 Hz, 2H), 3.61-3.50 (m, 2H), 2.82-2.66 (m, 1H), 1.91-1.71 (m, 6H), 1.05 (t, J=7.2 Hz, 3H). Mass (m/z): 367.5 [M+H]⁺.

### Compound 473

### 5-methyl-7-((2-methyl-4-(4-(trifluoromethyl)piperidin-1-yl)phenyl) amino)-2H-benzo[b][1,4]oxazin-3(4H)-one

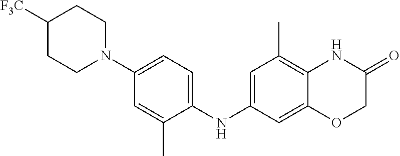 [SMILES: Cc1cc(N2CCC(C(F)(F)F)CC2)ccc1Nc1cc(C)c2c(c1)OCC(=O)N2]

The titled compound 473 (50.8 mg, 34%) as a purple solid was prepared according to the procedure outlined for compound 1. ¹H NMR (400 MHz, DMSO-d₆) δ 9.92 (s, 1H), 7.00 (s, 1H), 6.96 (d, J=8.8 Hz, 1H), 6.83 (d, J=2.8 Hz, 1H), 6.75 (dd, J=8.8, 2.8 Hz, 1H), 6.15 (s, 1H), 6.10 (s, 1H), 4.39 (s, 2H), 3.68 (d, J=12.0 Hz, 2H), 2.65 (t, J=11.6 Hz, 2H), 2.46-2.38 (m, 1H), 2.10 (s, 6H), 1.88 (d, J=11.6 Hz, 2H), 1.63-1.48 (m, 2H). Mass (m/z): 420.5[M+H]⁺.

### Compound 474

### 7-((6-(4-(trifluoromethyl)piperidin-1-yl)pyridin-3-yl)amino)-2H-benzo[b][1,4]oxazin-3(4H)-one

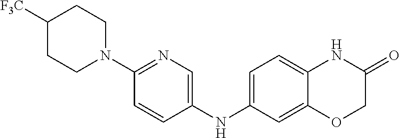 [SMILES: O=C1COc2cc(Nc3ccc(N4CCC(C(F)(F)F)CC4)nc3)ccc2N1]

The titled compound 474 (70.3 mg, 43%) as an off-white solid was prepared according to the procedure outlined for compound 1. ¹H NMR (400 MHz, DMSO-d₆) δ 10.41 (s, 1H), 7.93 (d, J=2.8 Hz, 1H), 7.61 (s, 1H), 7.34 (dd, J=9.2, 2.8 Hz, 1H), 6.84 (d, J=9.2 Hz, 1H), 6.70 (d, J=8.4 Hz, 1H), 6.49-6.39 (m, 2H), 4.47 (s, 2H), 4.27 (d, J=13.2 Hz, 2H), 2.76 (t, J=12.4 Hz, 2H), 2.61-2.54 (m, 1H), 1.85 (d, J=12.8 Hz, 2H), 1.52-1.37 (m, 2H). Mass (m/z): 393.4 [M+H]⁺.

### Compound 475

### 7-((2-(4-(trifluoromethyl)piperidin-1-yl)pyrimidin-5-yl)amino)-2H-benzo[b][1,4]oxazin-3(4H)-one

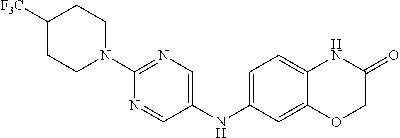 [SMILES: O=C1COc2cc(Nc3cnc(N4CCC(C(F)(F)F)CC4)nc3)ccc2N1]

The titled compound 475 (58.5 mg, 36%) as an off-white solid was prepared according to the procedure outlined for compound 1. ¹H NMR (400 MHz, DMSO-d₆) δ 10.42 (s, 1H), 8.22 (s, 2H), 7.57 (s, 1H), 6.70 (d, J=8.4 Hz, 1H), 6.44-6.35 (m, 2H), 4.72-4.64 (m, 2H), 4.47 (s, 2H), 2.94-2.82 (m, 2H), 2.59-2.55 (m, 1H), 1.87 (d, J=12.0 Hz, 2H), 1.46-1.31 (m, 2H). Mass (m/z): 394.4 [M+H]⁺.

### Compound 476

### 7-((2-(4-(trifluoromethyl)piperidin-1-yl)pyrimidin-5-yl)amino)-2H-benzo[b][1,4]oxazin-3(4H)-one

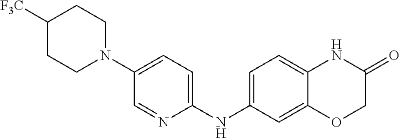 [SMILES: O=C1COc2cc(Nc3ccc(N4CCC(C(F)(F)F)CC4)cn3)ccc2N1]

The titled compound 476 (10.5 mg, 6%) as an off-white solid was prepared according to the procedure outlined for compound 1. ¹H NMR (400 MHz, DMSO-d₆) δ 10.42 (s, 1H), 8.22 (s, 2H), 7.57 (s, 1H), 6.70 (d, J=8.4 Hz, 1H), 6.44-6.35 (m, 2H), 4.72-4.64 (m, 2H), 4.47 (s, 2H), 2.94-2.82 (m, 2H), 2.59-2.55 (m, 1H), 1.87 (d, J=12.0 Hz, 2H), 1.46-1.31 (m, 2H). Mass (m/z): 393.4 [M+H]⁺.

### Compound 477

### 4,6-dimethyl-7-((2-methyl-4-(4-(trifluoromethyl)piperidin-1-yl)phenyl) amino)-2H-benzo[b][1,4]oxazin-3(4H)-one

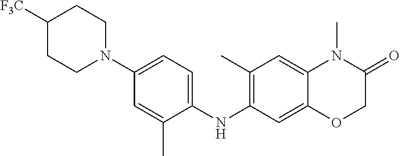 [SMILES: Cc1cc(N2CCC(C(F)(F)F)CC2)ccc1Nc1cc2c(cc1C)N(C)C(=O)CO2]

The titled compound 477 (67.7 mg, 44.7%) as an off-white solid was prepared according to the procedure outlined for compound 1. ¹H NMR (400 MHz, DMSO-d₆) δ 6.92-6.84 (m, 2H), 6.84-6.72 (m, 2H), 6.44 (s, 1H), 5.93 (s, 1H), 4.46 (s, 2H), 3.70 (d, J=12.4 Hz, 2H), 3.22 (s, 3H), 2.66 (t, J=12.0 Hz, 2H), 2.47-2.38 (m, 2H) 2.17 (s, 3H), 2.08 (s, 3H), 1.88 (d, J=13.2 Hz, 2H), 1.64-1.49 (m, 2H). Mass (m/z): 434.5 [M+H]⁺.

### Compound 478

### 7-((3-methoxy-4-(4-(trifluoromethyl)piperidin-1-yl)phenyl)amino)-4-methyl-2H-benzo[b][1,4]oxazin-3(4H)-one

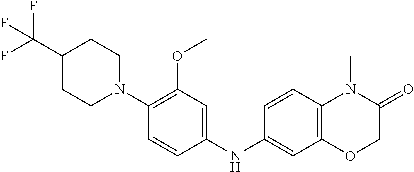 [SMILES: COc1cc(Nc2ccc3c(c2)OCC(=O)N3C)ccc1N1CCC(C(F)(F)F)CC1]

The titled compound 478 (23.3 mg, 14.7%) as a gray solid was prepared according to the procedure outlined for compound 1. ¹H NMR (400 MHz, DMSO-d₆) δ 7.97 (s, 1H), 7.62 (d, J=2.3 Hz, 1H), 7.04-6.95 (m, 2H), 6.69 (dd, J=8.8, 2.4 Hz, 1H), 6.58 (d, J=2.4 Hz, 1H), 4.58 (s, 2H), 3.85-3.78 (m, 2H), 3.77 (s, 3H), 3.23 (s, 3H), 2.66 (d, J=24.0 Hz, 2H), 1.85 (d, J=12.0 Hz, 2H), 1.57 (qd, J=12.4, 3.6 Hz, 2H). Mass (m/z): 436.2 [M+H]⁺.

### Compound 479

### 7-((4-(2-methyltetrahydro-2H-pyran-4-yl)phenyl)amino)-2H-benzo[b][1,4]oxazin-3(4H)-one

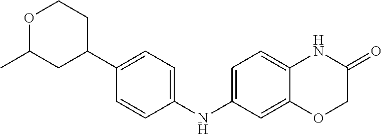 [SMILES: CC1CC(c2ccc(Nc3ccc4c(c3)OCC(=O)N4)cc2)CCO1]

The titled compound 479 (26.2 mg, 11.4%) as a white solid was prepared according to the procedure outlined for compound 1. ¹H NMR (400 MHz, DMSO-d₆) δ 10.50 (s, 1H), 7.94 (s, 1H), 7.13-7.05 (m, 2H), 7.03-6.88 (m, 2H), 6.75 (d, J=8.4 Hz, 1H), 6.69-6.55 (m, 2H), 4.50 (s, 2H), 4.03-3.85 (m, 1H), 3.54-3.41 (m, 2H), 2.66 (dd, J=24.0, 3.6 Hz, 1H), 1.82-1.45 (m, 31H), 1.25 (td, J=12.4, 10.8 Hz, 1H), 1.12 (d, J=6.0 Hz, 3H). Mass (m/z): 339.2 [M+H]⁺.

### Compound 480

### 5-fluoro-7-((2-methyl-4-(4-(trifluoromethyl)piperidin-1-yl)phenyl) amino)-2H-benzo[b][1,4]oxazin-3(4H)-one

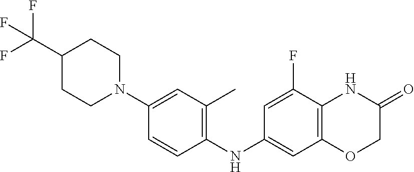 [SMILES: Cc1cc(N2CCC(C(F)(F)F)CC2)ccc1Nc1cc(F)c2c(c1)OCC(=O)N2]

The titled compound 480 (25 mg, 5.9%) as a white solid was prepared according to the procedure outlined for compound 1. ¹H NMR (400 MHz, DMSO-d₆) δ 10.50 (s, 1H), 7.34 (s, 1H), 6.98 (d, J=8.4 Hz, 1H), 6.86 (d, J=2.8 Hz, 1H), 6.78 (dd, J=8.8, 2.8 Hz, 1H), 6.06 (d, J=11.2 Hz, 2H), 4.48 (s, 2H), 3.72 (d, J=12.4 Hz, 2H), 2.67 (t, J=12.0 Hz, 3H), 2.51-2.49 (m, 1H), 2.10 (s, 3H), 1.88 (d, J=12.0 Hz, 2H), 1.56 (qd, J=12.4, 4.0 Hz, 2H). Mass (m/z): 424.2 [M+H]⁺.

### Compound 481

### 5-fluoro-7-((3-methyl-4-(4-(trifluoromethyl)piperidin-1-yl)phenyl) amino)-2H-benzo[b][1,4]oxazin-3(4H)-one

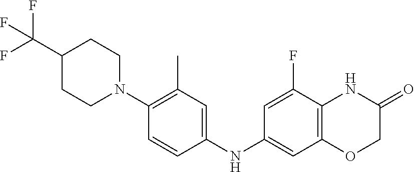 [SMILES: Cc1cc(Nc2cc(F)c3c(c2)OCC(=O)N3)ccc1N1CCC(C(F)(F)F)CC1]

The titled compound 481 (34.7 mg, 8.2%) as a white solid was prepared according to the procedure outlined for compound 1. ¹H NMR (400 MHz, DMSO-d₆) δ 10.60 (s, 1H), 7.98 (s, 1H), 6.95 (d, J=8.8 Hz, 1H), 6.86 (d, J=7.2 Hz, 2H), 6.44 (d, J=12.4 Hz, 1H), 6.38 (s, 1H), 4.53 (s, 2H), 3.05 (d, J=11.6 Hz, 2H), 2.63 (d, J=11.6 Hz, 2H), 2.51-2.49 (m, 1H), 2.20 (s, 3H), 1.97-1.80 (m, 2H), 1.60 (qd, J=12.4, 4.0 Hz, 2H). Mass (m/z): 424.2 [M+H]⁺.

### Compound 482

### 2-methyl-7-((2-methyl-4-(4-(trifluoromethyl)piperidin-1-yl)phenyl) amino)-2H-benzo[b][1,4]oxazin-3(4H)-one

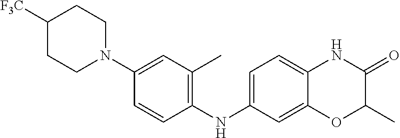 [SMILES: Cc1cc(N2CCC(C(F)(F)F)CC2)ccc1Nc1ccc2c(c1)OC(C)C(=O)N2]

The titled compound 482 (27.2 mg, 13.1%) as a white solid was prepared according to the procedure outlined for compound 1. ¹H NMR (400 MHz, DMSO-d₆) δ 10.29 (s, 1H), 7.05 (s, 1H), 6.96 (d, J=8.8 Hz, 1H), 6.84 (d, J=2.8 Hz, 1H), 6.75 (dd, J=8.0, 2.8 Hz, 1H), 6.65 (d, J=8.4 Hz, 1H), 6.29 (d, J=8.4 Hz, 1H), 6.23 (s, 1H), 4.52 (q, J=6.8 Hz, 1H), 3.68 (d, J=12.0 Hz, 2H), 2.65 (t, J=12.0 Hz, 2H), 2.47-2.37 (m, 1H), 2.11 (s, 3H), 1.88 (d, J=12.4 Hz, 2H), 1.61-1.51 (m, 2H), 1.36 (d, J=6.8 Hz, 3H). Mass (m/z): 420.2 [M+H]⁺.

### Compound 483

### 2-ethyl-7-((2-methyl-4-(4-(trifluoromethyl)piperidin-1-yl)phenyl) amino)-2H-benzo[b][1,4]oxazin-3(4H)-one

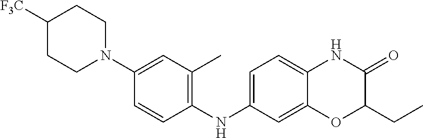 [SMILES: CCC1Oc2cc(Nc3ccc(N4CCC(C(F)(F)F)CC4)cc3C)ccc2NC1=O]

The titled compound 483 (12 mg, 5.9%) as a white solid was prepared according to the procedure outlined for 1. ¹H NMR (400 MHz, DMSO-d₆) δ 10.29 (s, 1H), 7.05 (s, 1H), 6.97 (d, J=8.8 Hz, 1H), 6.85 (d, J=2.8 Hz, 1H), 6.75 (d, J=8.0 Hz, 1H), 6.66 (d, J=8.4 Hz, 1H), 6.29 (d, J=8.4 Hz, 1H), 6.23 (s, 1H), 4.36 (q, J=8.0 Hz, 1H), 3.68 (d, J=12.0 Hz, 2H), 2.63 (t, J=12.4 Hz, 2H), 2.47-2.37 (m, 1H), 2.11 (s, 3H), 1.88 (d, J=12.8 Hz, 2H), 1.78-1.67 (m, 2H), 1.61-1.50 (m, 2H), 0.95 (t, J=7.2 Hz, 3H). Mass (m/z): 434.2 [M+H]⁺.

### Compound 484

### 7-((4-((2R,4R)-2-methyltetrahydro-2H-pyran-4-yl)phenyl)amino)-2H-benzo[b][1,4]oxazin-3(4H)-one

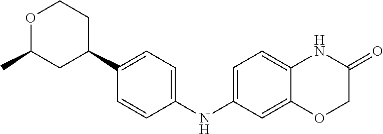 [SMILES: C[C@@H]1C[C@H](c2ccc(Nc3ccc4c(c3)OCC(=O)N4)cc2)CCO1]

The titled compound 484 (26.2 mg, 11.4%) as a white solid was prepared according to the procedure outlined for 1. ¹H NMR (400 MHz, DMSO-d₆) δ 10.50 (s, 1H), 7.94 (s, 1H), 7.13-7.05 (m, 2H), 7.03-6.88 (m, 2H), 6.75 (d, J=8.4 Hz, 1H), 6.69-6.55 (m, 2H), 4.50 (s, 2H), 4.03-3.85 (m, 1H), 3.54-3.41 (m, 2H), 2.66 (dd, J=24.0, 3.6 Hz, 1H), 1.82-1.45 (m, 3H), 1.25 (td, J=12.8, 10.8 Hz, 1H), 1.12 (d, J=6.0 Hz, 3H). Mass (m/z): 339.2 [M+H]⁺.

### Compound 485

### 7-((3-methoxy-4-(4-(trifluoromethyl)piperidin-1-yl)phenyl)amino)-2H-benzo[b][1,4]oxazin-3(4H)-one

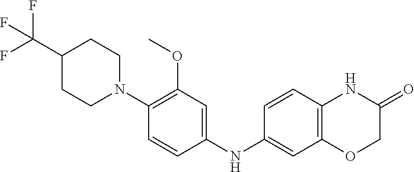 [SMILES: COc1cc(Nc2ccc3c(c2)OCC(=O)N3)ccc1N1CCC(C(F)(F)F)CC1]

The titled compound 485 (32 mg, 20.7%) as a white solid was prepared according to the procedure outlined for compound 1. ¹H NMR (400 MHz, DMSO-d₆) δ 10.46 (s, 1H), 7.81 (s, 1H), 6.76 (dd, J=19.6, 8.4 Hz, 2H), 6.66-6.49 (m, 4H), 4.49 (s, 2H), 3.73 (s, 3H), 3.28 (s, 2H), 2.55-2.53 (m, 2H), 2.51-2.49 (m, 1H), 1.85 (d, J=12.8 Hz, 2H), 1.59 (qd, J=12.0, 40 Hz, 2H). Mass (m/z): 424.4 [M+H]⁺.

### Compound 486

### 7-((3-fluoro-4-(4-(trifluoromethyl)piperidin-1-yl)phenyl)amino)-2H-benzo[b][1,4]oxazin-3(4H)-one

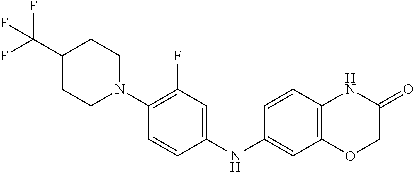 [SMILES: O=C1COc2cc(Nc3ccc(N4CCC(C(F)(F)F)CC4)c(F)c3)ccc2N1]

The titled compound 486 (18.1 mg, 11.6%) as a pink solid was prepared according to the procedure outlined for compound 1. ¹H NMR (400 MHz, DMSO-d₆) δ 10.52 (s, 1H), 8.00 (s, 1H), 6.94 (t, J=9.2 Hz, 1H), 6.82-6.70 (m, 3H), 6.68-6.55 (m, 2H), 4.51 (s, 2H), 3.27 (d, J=12.0 Hz, 2H), 2.71-2.60 (m, 2H), 2.44-2.36 (m, 1H), 1.88 (d, J=12.4 Hz, 2H), 1.70-1.51 (m, 2H). Mass (m/z): 410.4 [M+H]⁺.

### Compound 487

### 7-((2,3-dimethyl-4-(4-(trifluoromethyl)piperidin-1-yl)phenyl)amino)-2H-benzo[b][1,4]oxazin-3(4H)-one

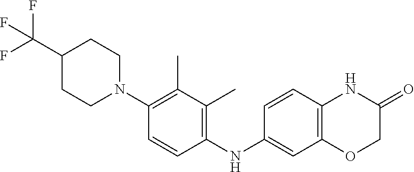 [SMILES: Cc1c(Nc2ccc3c(c2)OCC(=O)N3)ccc(N2CCC(C(F)(F)F)CC2)c1C]

The titled compound 487 (10 mg, 6.5%) as a pink solid was prepared according to the procedure outlined for compound 1. ¹H NMR (400 MHz, DMSO-d₆) δ 10.38 (s, 1H), 7.21 (s, 1H), 6.98-6.81 (m, 2H), 6.66 (d, J=8.4 Hz, 1H), 6.33-6.16 (m, 2H), 4.44 (s, 2H), 3.03 (d, J=11.6 Hz, 2H), 2.63 (d, J=11.6 Hz, 3H), 2.19 (s, 3H), 2.05 (s, 3H), 1.89 (d, J=12.4 Hz, 2H), 1.64 (tt, J=14.8, 8.0 Hz, 2H). Mass (m/z): 420.2 [M+H]⁺.

### Compound 488

### 7-((5-fluoro-2-methyl-4-(4-(trifluoromethyl)piperidin-1-yl)phenyl) amino)-2H-benzo[b][1,4]oxazin-3(4H)-one

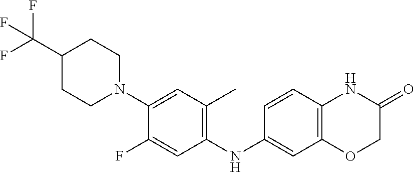 [SMILES: Cc1cc(N2CCC(C(F)(F)F)CC2)c(F)cc1Nc1ccc2c(c1)OCC(=O)N2]

The titled compound 488 (24 mg, 15.7%) as a pink solid was prepared according to the procedure outlined for compound 1. ¹H NMR (400 MHz, DMSO-d₆) δ 10.47 (s, 1H), 7.22 (s, 1H), 6.94-6.68 (m, 3H), 6.53-6.35 (m, 2H), 4.49 (s, 2H), 3.29 (d, J=11.6 Hz, 2H), 2.74-2.59 (m, 2H), 2.42-2.39 (m, 1H), 2.11 (s, 3H), 1.89 (d, J=12.4 Hz, 2H), 1.60 (q, J=12.4, 11.6 Hz, 2H). Mass (m/z): 424.2 [M+H]⁺.

### Compound 489

### 7-((2-methyl-6-(4-(trifluoromethyl)piperidin-1-yl)pyridin-3-yl) amino)-2H-benzo[b][1,4]oxazin-3(4H)-one

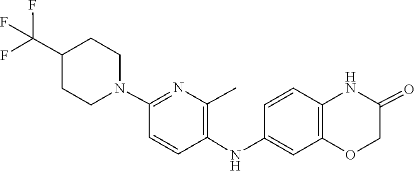 [SMILES: Cc1nc(N2CCC(C(F)(F)F)CC2)ccc1Nc1ccc2c(c1)OCC(=O)N2]

The titled compound 489 (26.7 mg, 17%) as a yellow solid was prepared according to the procedure outlined for compound 1. ¹H NMR (400 MHz, DMSO-d₆) δ 10.38 (s, 1H), 7.28 (d, J=8.8 Hz, 1H), 7.13 (s, 1H), 6.67 (t, J=9.6 Hz, 2H), 6.19 (d, J=9.6 Hz, 2H), 4.45 (s, 2H), 4.34 (d, J=13.2 Hz, 2H), 2.76 (t, J=12.8 Hz, 2H), 2.42-2.39 (m, 1H), 2.21 (s, 3H), 1.86 (d, J=12.8 Hz, 2H), 1.43 (qd, J=12.4, 3.6 Hz, 2H). Mass (m/z): 407.2 [M+H]⁺.

### Compound 490

### 4-methyl-7-((2-methyl-4-(4-methylpiperidin-1-yl)phenyl)amino)-2H-benzo[b][1,4]oxazin-3(4H)-one

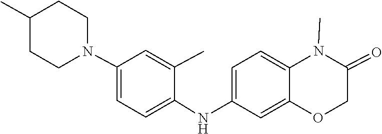 [SMILES: Cc1cc(N2CCC(C)CC2)ccc1Nc1ccc2c(c1)OCC(=O)N2C]

The titled compound 490 (32.9 mg, 18.35%) as a white solid was prepared according to the procedure outlined for compound 1. ¹H NMR (400 MHz, DMSO-d₆) δ 7.18 (s, 1H), 6.96 (d, J=8.8 Hz, 1H), 6.90 (d, J=8.8 Hz, 1H), 6.82 (d, J=2.8 Hz, 1H), 6.73 (dd, J=8.8, 2.4 Hz, 1H), 6.33 (dd, J=8.8, 2.4 Hz, 1H), 6.25 (d, J=2.4 Hz, 1H), 4.53 (s, 2H), 3.57 (d, J=12.4 Hz, 2H), 3.20 (s, 3H), 2.58 (t, J=11.2 Hz, 2H), 2.09 (d, J=11.2 Hz, 3H), 1.68 (d, J=11.6 Hz, 2H), 1.53-1.37 (m, 1H), 1.23 (qd, J=12.4, 4.0 Hz, 2H), 0.94 (d, J=6.8 Hz, 3H). Mass (m/z): 365.9 [M+H]⁺.

### Compound 491

### 7-((2-methyl-4-(4-methylpiperidin-1-yl)phenyl)amino)-2H-benzo[b][1,4]oxazin-3(4H)-one

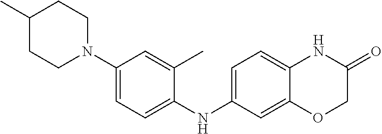 [SMILES: Cc1cc(N2CCC(C)CC2)ccc1Nc1ccc2c(c1)OCC(=O)N2]

The titled compound 491 (7.8 mg, 4.52%) as a white solid was prepared according to the procedure outlined for compound 1. ¹H NMR (400 MHz, CDCl₃) δ 7.95 (s, 1H), 7.05 (d, J=8.0 Hz, 1H), 6.81 (d, J=20.0 Hz, 2H), 6.61 (d, J=8.0 Hz, 1H), 6.36 (s, 2H), 5.12 (s, 1H), 4.55 (s, 2H), 3.60 (d, J=11.2 Hz, 2H), 2.67 (s, 2H), 2.19 (s, 3H), 1.75 (d, J=12.4 Hz, 2H), 1.58 (s, 1H), 1.36 (d, J=11.2 Hz, 2H), 0.99 (d, J=6.4 Hz, 3H). Mass (m/z): 352.1 [M+H]⁺.

### Compound 492

### 2-methyl-3-((4-methyl-3-oxo-3,4-dihydro-2H-benzo[b][1,4]oxazin-7-yl) amino)-6-(4-(trifluoromethyl)piperidin-1-yl)benzonitrile

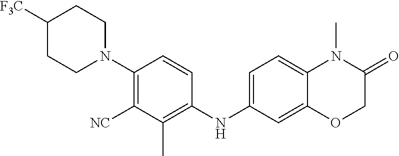 [SMILES: Cc1c(Nc2ccc3c(c2)OCC(=O)N3C)ccc(N2CCC(C(F)(F)F)CC2)c1C#N]

The titled compound 492 (74.9 mg, 59.73%) as a white solid was prepared according to the procedure outlined for compound 1. ¹H NMR (400 MHz, CDCl₃) δ 7.31-7.26 (m, 1H), 6.94-6.86 (m, 1H), 6.83 (d, J=8.6 Hz, 1H), 6.46 (dd, J=11.8, 2.9 Hz, 2H), 4.57 (s, 2H), 3.55 (d, J=11.8 Hz, 2H), 3.32 (s, 3H), 2.75 (d, J=11.4 Hz, 2H), 2.42 (s, 3H), 2.23-2.09 (m, 1H), 2.04-1.86 (m, 4H). Mass (m/z): 444.8 [M+H]⁺.

### Compound 493

### N-(4-((2R,6S)-2,6-dimethylmorpholino)-2-methylphenyl)-1H-benzo[d] imidazol-5-amine

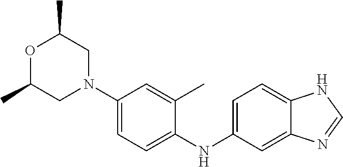 [SMILES: Cc1cc(N2C[C@@H](C)O[C@@H](C)C2)ccc1Nc1ccc2[nH]cnc2c1]

The titled compound 493 (35.6 mg, 56%) as a grey solid was prepared according to the procedure outlined for compound 1. ¹H NMR (400 MHz, DMSO-d₆) δ 11.95 (s, 1H), 7.94 (s, 1H), 7.36 (d, J=8.8 Hz, 1H), 7.06-6.99 (m, 2H), 6.84 (d, J=2.4 Hz, 1H), 6.77-6.70 (m, 2H), 6.67 (s, 1H), 3.69 (m, 2H), 3.49 (d, J=10.4 Hz, 2H), 2.25-2.17 (m, 2H), 2.15 (s, 3H), 1.15 (d, J=6.4 Hz, 6H). Mass (m/z): 337 [M+H]⁺.

### Compound 494

### 6-((4-((2R,6S)-2,6-dimethylmorpholino)-2-methylphenyl)amino)-3,4-dihydroquinazolin-2(1H)-one

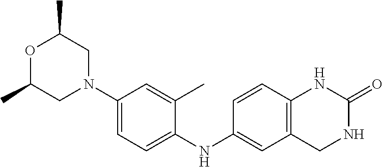 [SMILES: Cc1cc(N2C[C@@H](C)O[C@@H](C)C2)ccc1Nc1ccc2c(c1)CNC(=O)N2]

The titled compound 494 (4.2 mg, 1.72%) as a light-yellow solid was prepared according to the procedure outlined for compound 461. ¹H NMR (400 MHz, DMSO-d₆) δ 8.69 (s, 1H), 6.92 (d, J=8.8 Hz, 1H), 6.84 (s, 1H), 6.79 (d, J=2.8 Hz, 1H), 6.70 (dd, J=8.8, 2.8 Hz, 1H), 6.57 (d, J=8.4 Hz, 2H), 6.50 (dd, J=8.4, 2.4 Hz, 1H), 6.44 (s, 1H), 4.18 (s, 2H), 3.71-3.64 (m, 2H), 3.46 (d, J=10.4 Hz, 2H), 2.21-2.15 (m, 2H), 2.11 (s, 3H), 1.14 (d, J=6.2 Hz, 6H). Mass (m/z): 366.9 [M+H]⁺.

### Compound 495

### 6-hydroxy-4-methyl-7-({4-[4-(trifluoromethyl)piperidin-1-yl]phenyl}amino)-2H-1,4-benzoxazin-3-one

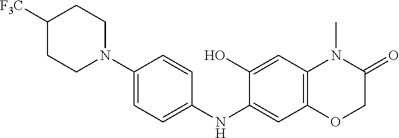 [SMILES: CN1C(=O)COc2cc(Nc3ccc(N4CCC(C(F)(F)F)CC4)cc3)c(O)cc21]

The titled compound 495 (15 mg, 7.6%) as a yellow solid was prepared according to the procedure outlined for compound 1. ¹H NMR (400 MHz, DMSO-d₆) δ 9.44 (s, 1H), 7.72 (s, 1H), 7.39 (s, 2H), 6.93 (d, J=7.6 Hz, 2H), 6.73 (d, J=33.2 Hz, 2H), 4.55 (s, 2H), 3.63 (s, 4H), 3.23 (s, 3H), 2.76 (d, J=23.6 Hz, 1H), 2.12 (s, 2H), 1.94 (d, J=18.0 Hz, 2H). Mass (m/z): 421.8[M+H]⁺.

### Compound 496

### 6-((2-methyl-4-(4-(trifluoromethyl)piperidin-1-yl)phenyl)amino) benzo[d]oxazol-2(3H)-one

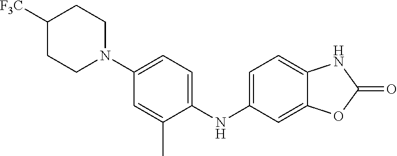 [SMILES: Cc1cc(N2CCC(C(F)(F)F)CC2)ccc1Nc1ccc2[nH]c(=O)oc2c1]

The titled compound 496 (15 mg, 4.9%) as a yellow solid was prepared according to the procedure outlined for compound 1. ¹H NMR (400 MHz, CD₃OD) δ 7.03 (d, J=8.8 Hz, 1H), 6.91 (d, J=2.4 Hz, 1H), 6.82-6.84 (m, 2H), 6.60-6.52 (m, 2H), 3.68 (d, J=12.4 Hz, 2H), 2.67-2.69 (m, 2H), 2.34-2.23 (m, 1H), 2.17 (d, J=5.2 Hz, 3H), 2.03-1.94 (m, 2H), 1.77-1.66 (m, 2H). Mass (m/z): 392.2 [M+H]⁺.

### Compound 497

### N-(2-methyl-4-(4-(trifluoromethyl)piperidin-1-yl)phenyl)-1H-indazol-5-amine

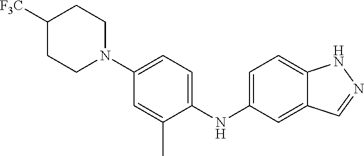 [SMILES: Cc1cc(N2CCC(C(F)(F)F)CC2)ccc1Nc1ccc2[nH]ncc2c1]

The titled compound 497 (41.8 mg, 46.22%) as a yellow solid was prepared according to the procedure outlined for compound 1. ¹H NMR (400 MHz, CD₃OD) δ 7.79 (s, 1H), 7.40 (d, J=8.9 Hz, 1H), 7.10 (dd, J=8.8, 2.0 Hz, 1H), 7.05 (d, J=8.8 Hz, 1H), 6.93 (s, 2H), 6.83 (s, 1H), 3.67 (d, J=12.4 Hz, 2H), 2.74-2.63 (m, 2H), 2.38-2.24 (m, 1H), 2.23 (s, 3H), 1.99 (d, J=12.8 Hz, 2H), 1.75 (qd, J=12.8, 4.0 Hz, 2H). Mass (m/z): 374.7 [M+H]⁺.

### Compound 498

### 7-((6-(2-ethylmorpholino)-2-methylpyridin-3-yl)amino)-2H-benzo[b][1,4]oxazin-3(4H)-one

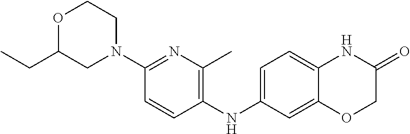 [SMILES: CCC1CN(c2ccc(Nc3ccc4c(c3)OCC(=O)N4)c(C)n2)CCO1]

The titled compound 498 (36.9 mg, 51.3%) as a light-yellow solid was prepared according to the procedure outlined for compound 1. ¹H NMR (400 MHz, DMSO-d₆) δ 10.36 (s, 1H), 7.29 (d, J=8.4 Hz, 1H), 7.13 (s, 1H), 6.65 (d, J=8.4 Hz, 2H), 6.26-6.11 (m, 2H), 4.45 (s, 2H), 4.04 (d, J=12.4 Hz, 1H), 3.99-3.89 (m, 2H), 3.60-3.47 (m, 1H), 3.42-3.33 (m, 1H), 2.80-2.66 (m, 1H), 2.43 (t, J=11.6 Hz, 1H), 2.22 (s, 3H), 1.56-1.46 (m, 2H), 0.94 (t, J=8.4, 6.4 Hz, 3H). Mass (m/z): 369.2 [M+H]⁺.

### Compound 499

### 7-((6-((2S,6R)-2,6-diethylmorpholino)-2-methylpyridin-3-yl) amino)-2H-benzo[b][1,4]oxazin-3(4H)-one

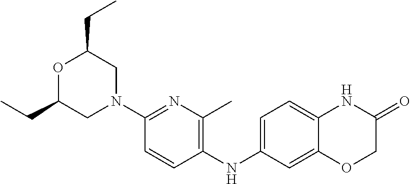 [SMILES: CC[C@@H]1CN(c2ccc(Nc3ccc4c(c3)OCC(=O)N4)c(C)n2)C[C@H](CC)O1]

The titled compound 499 (34.5 mg, 50.6%) as a white solid was prepared according to the procedure outlined for compound 1. ¹H NMR (400 MHz, DMSO-d₆) δ 10.36 (s, 1H), 7.28 (d, J=8.8 Hz, 1H), 7.12 (s, 1H), 6.66 (d, J=8.0 Hz, 2H), 6.23-6.11 (m, 2H), 4.44 (s, 2H), 4.06 (d, J=12.4 Hz, 2H), 3.43-3.34 (m, 2H), 2.34 (t, J=11.2 Hz, 2H), 2.21 (s, 3H), 1.58-1.45 (m, 4H), 0.96 (t, J=6.4 Hz, 6H). Mass (m/z): 397.3 [M+H]⁺.

### Compound 500

### 7-((6-(2,2-dimethylmorpholino)-2-methylpyridin-3-yl)amino)-2H-benzo[b][1,4]oxazin-3(4H)-one

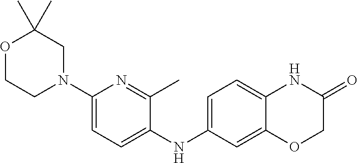 [SMILES: Cc1nc(N2CCOC(C)(C)C2)ccc1Nc1ccc2c(c1)OCC(=O)N2]

The titled compound 500 (5.9 mg, 3.54%) as a yellow solid was prepared according to the procedure outlined for compound 1. ¹H NMR (400 MHz, DMSO-d₆) δ 10.31 (s, 1H), 7.23 (d, J=8.8 Hz, 1H), 7.07 (s, 1H), 6.60 (d, J=8.4 Hz, 2H), 6.13 (d, J=8.8 Hz, 2H), 4.39 (s, 2H), 3.66 (t, J=5.2 Hz, 2H), 3.18 (s, 2H), 2.15 (s, 3H), 1.15 (s, 6H). Mass (m/z): 369.6 [M+H]⁺.

### Compound 501

### 7-((4-(diethylamino)-2-methylphenyl)amino)-2H-benzo[b][1,4]oxazin-3(4H)-one

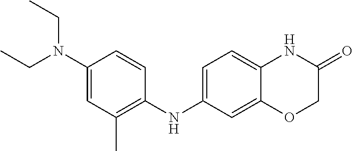 [SMILES: CCN(CC)c1ccc(Nc2ccc3c(c2)OCC(=O)N3)c(C)c1]

The titled compound 501 (33.4 mg, 22.04%) as a white solid was prepared according to the procedure outlined for compound 1. ¹H NMR (400 MHz, DMSO-d₆) δ 10.32 (s, 1H), 6.96 (s, 1H), 6.89 (d, J=8.8 Hz, 1H), 6.62 (d, J=8.8 Hz, 1H), 6.54 (d, J=3.2 Hz, 1H), 6.48 (dd, J=8.8, 2.8 Hz, 1H), 6.18 (dd, J=8.4, 2.4 Hz, 1H), 6.14 (d, J=2.4 Hz, 1H), 4.42 (d, J=2.0 Hz, 2H), 3.27 (t, J=7.2 Hz, 4H), 2.08 (s, 3H), 1.07 (t, J=7.2 Hz, 6H). Mass (m/z): 326.3 [M+H]⁺.

### Compound 502

### 7-((2-methyl-4-(3-(trifluoromethyl)pyrrolidin-1-yl)phenyl)amino)-2H-benzo[b][1,4]oxazin-3(4H)-one

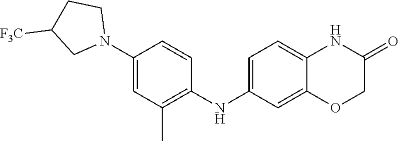 [SMILES: Cc1cc(N2CCC(C(F)(F)F)C2)ccc1Nc1ccc2c(c1)OCC(=O)N2]

The titled compound 502 (3.9 mg, 4.35%) as a white solid was prepared according to the procedure outlined for compound 1. ¹H NMR (400 MHz, DMSO-d₆) δ 10.33 (s, 1H), 7.03 (s, 1H), 6.93 (d, J=8.4 Hz, 1H), 6.62 (d, J=8.5 Hz, 1H), 6.52 (s, 1H), 6.44 (d, J=8.3 Hz, 1H), 6.18 (d, J=8.5 Hz, 1H), 6.14 (s, 1H), 4.43 (s, 2H), 3.49 (t, J=8.5 Hz, 1H), 3.26 (d, J=8.0 Hz, 4H), 2.25 (d, J=6.3 Hz, 1H), 2.10 (s, 4H). Mass (m/z): 437.7 [M+H]⁺. Mass (m/z): 392.7 [M+H]⁺.

### Compound 503

### 7-((6-(4-methoxy-4-(trifluoromethyl)piperidin-1-yl)-2-methylpyridin-3-yl) amino)-2H-benzo[b][1,4]oxazin-3(4H)-one

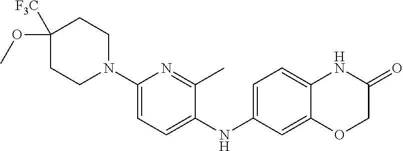 [SMILES: COC1(C(F)(F)F)CCN(c2ccc(Nc3ccc4c(c3)OCC(=O)N4)c(C)n2)CC1]

The titled compound 503 (10.7 mg, 12.78%) as a white solid was prepared according to the procedure outlined for compound 1. ¹H NMR (400 MHz, DMSO-d₆) δ 10.37 (s, 1H), 7.29 (d, J=8.7 Hz, 1H), 7.13 (s, 1H), 6.70 (d, J=8.7 Hz, 1H), 6.66 (d, J=8.2 Hz, 1H), 6.19 (d, J=10.7 Hz, 2H), 4.45 (s, 2H), 4.18 (d, J=13.1 Hz, 2H), 3.41 (s, 3H), 2.89 (t, J=12.8 Hz, 2H), 2.22 (s, 3H), 1.98 (d, J=13.7 Hz, 2H), 1.70 (td, J=13.3, 4.5 Hz, 2H). Mass (m/z): 437.7 [M+H]⁺.

### Compound 504

### 7-((2-methyl-6-(4-(trifluoromethyl)piperidin-1-yl)pyridin-3-yl)amino)-2H-benzo[b][1,4]oxazin-3(4H)-one

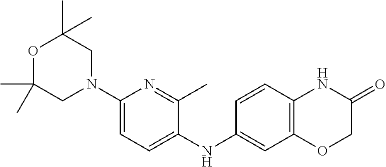 [SMILES: Cc1nc(N2CC(C)(C)OC(C)(C)C2)ccc1Nc1ccc2c(c1)OCC(=O)N2]

The titled compound 504 (18.9 mg, 15.5%) as a yellow solid was prepared according to the procedure outlined for compound 1. ¹H NMR (400 MHz, DMSO-d₆) δ 10.38 (s, 1H), 7.27 (d, J=8.8 Hz, 1H), 7.12 (s, 1H), 6.65 (t, J=8.0 Hz, 2H), 6.18 (d, J=10.0 Hz, 2H), 4.44 (s, 2H), 2.20 (s, 3H), 1.19 (s, 12H). Mass (m/z): 397.3 [M+H]⁺.

### Compound 505

### 7-((2-methyl-6-(4-(trifluoromethyl)piperidin-1-yl)pyridin-3-yl)amino)-2H-benzo[b][1,4]oxazin-3(4H)-one

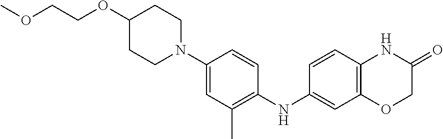 [SMILES: COCCOC1CCN(c2ccc(Nc3ccc4c(c3)OCC(=O)N4)c(C)c2)CC1]

The titled compound 505 (9.2 mg, 9.4%) as a yellow solid was prepared according to the procedure outlined for compound 1. ¹H NMR (400 MHz, DMSO-d₆) δ 10.39 (s, 1H), 7.09 (s, 1H), 6.94 (d, J=8.4 Hz, 1H), 6.81 (s, 1H), 6.75-6.63 m, 3H), 6.29-6.21 (m, 2H), 4.44 (s, 2H), 3.56 (t, J=5.2 Hz, 2H), 3.44 (t, J=4.8 Hz, 4H), 3.40 (s, 1H), 3.25 (s, 3H), 2.79 (t, J=11.2 Hz, 2H), 2.10 (s, 3H), 1.94-1.89 (m, 2H), 1.45-1.53 (q, J=6.6, 3.6 Hz, 2H). Mass (m/z): 412.2 [M+H]⁺.

### Compound 506

### 4-methyl-7-((2-methyl-4-(piperidin-1-yl)phenyl)amino)-2H-benzo[b][1,4]oxazin-3(4H)-oneyl)phenyl)amino)-2H-benzo[b][1,4]oxazin-3(4H)-one

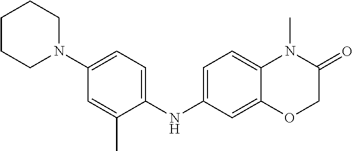 [SMILES: Cc1cc(N2CCCCC2)ccc1Nc1ccc2c(c1)OCC(=O)N2C]

The titled compound 506 (46.7 mg, 31.5%) as a yellow solid was prepared according to the procedure outlined for compound 1. ¹H NMR (400 MHz, CD₃OD) δ 7.03 (d, J=8.8 Hz, 1H), 6.94-6.87 (m, 2H), 6.82 (d, J=8.8 Hz, 1H), 6.40 (d, J=8.8 Hz, 1H), 6.30 (s, 1H), 4.50 (s, 2H), 3.30 (s, 3H), 3.11-3.04 (m, 4H), 2.16 (s, 3H), 1.78-1.68 (m, 4H), 1.63-1.54 (m, 3H). Mass (m/z): 352.5 [M+H]⁺.

### Compound 507

### 7-((4-(4-methylpiperidin-1-yl)phenyl)amino)-2H-benzo[b][1,4]oxazin-3(4H)-one

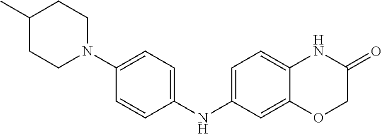 [SMILES: CC1CCN(c2ccc(Nc3ccc4c(c3)OCC(=O)N4)cc2)CC1]

The titled compound 507 (34.1 mg, 19.5%) as a purple solid was prepared according to the procedure outlined for compound 1. ¹H NMR (400 MHz, DMSO-d₆) δ 10.43 (s, 1H), 7.65 (s, 1H), 6.95-6.81 (m, 4H), 6.70 (dd, J=8.4, 2.4 Hz, 1H), 6.55-6.46 (m, 2H), 4.47 (s, 2H), 3.53-3.45 (m, 2H), 2.59-2.52 (m, 2H), 1.68 (d, J=12.8 Hz, 2H), 1.49-1.39 (m, 1H), 1.32-1.17 (m, 2H), 0.97-0.90 (m, 3H). Mass (m/z): 337.5 [M+H]⁺.

### Compound 508

### 7-((4-(4-fluoropiperidin-1-yl)phenyl)amino)-2H-benzo[b][1,4]oxazin-3(4H)-one

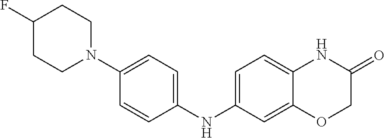 [SMILES: O=C1COc2cc(Nc3ccc(N4CCC(F)CC4)cc3)ccc2N1]

The titled compound 508 (42.5 mg, 24%) as a grey solid was prepared according to the procedure outlined for compound 1. ¹H NMR (400 MHz, CD₃OD) δ 7.02-6.91 (m, 4H), 6.75-6.68 (m, 1H), 6.60-6.53 (m, 2H), 4.83-4.60 (m, 1H), 4.49 (s, 2H), 3.27-3.20 (m, 2H), 3.06-3.01 (m, 2H), 2.08-1.96 (m, 2H), 1.96-1.89 (m, 2H). Mass (m/z): 342.5 [M+H]⁺.

### Compound 509

### 7-((4-(4-hydroxy-4-(trifluoromethyl)piperidin-1-yl)-2-methylphenyl) amino)-2H-benzo[b][1,4]oxazin-3(4H)-one

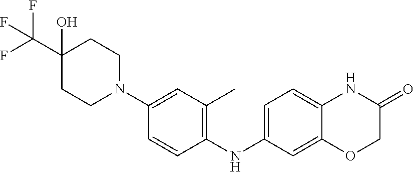 [SMILES: Cc1cc(N2CCC(O)(C(F)(F)F)CC2)ccc1Nc1ccc2c(c1)OCC(=O)N2]

The titled compound 509 (38 mg, 23.5%) as an off-white solid was prepared according to the procedure outlined for compound 1. ¹H NMR (400 MHz, CD₃OD) δ 7.03 (d, J=8.8 Hz, 1H), 6.91 (s, 1H), 6.83 (d, J=8.8 Hz, 1H), 6.67 (d, J=8.4 Hz, 1H), 6.33 (d, J=8.8 Hz, 1H), 6.29 (s, 1H), 4.47 (s, 2H), 3.49 (d, J=12.0 Hz, 2H), 2.98 (t, J=12.4 Hz, 2H), 2.17 (s, 3H), 2.02-1.90 (m, 2H), 1.81 (d, J=13.2 Hz, 2H). Mass (m/z): 422.5 [M+H]⁺.

### Compound 510

### 7-((4-(4-hydroxy-4-(trifluoromethyl)piperidin-1-yl)phenyl)amino)-2H-benzo[b][1,4]oxazin-3(4H)-one

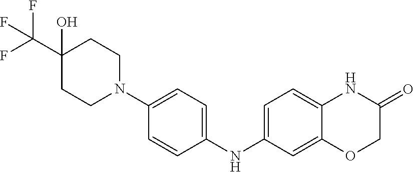 [SMILES: O=C1COc2cc(Nc3ccc(N4CCC(O)(C(F)(F)F)CC4)cc3)ccc2N1]

The titled compound 510 (56.7 mg, 36%) as an off-white solid was prepared according to the procedure outlined for compound 1. ¹H NMR (400 MHz, CD₃OD) δ 7.00-6.96 (m, 4H), 6.75-6.69 (m, 1H), 6.60-6.56 (m, 2H), 4.49 (s, 2H), 3.45-3.38 (m, 2H), 2.98-2.94 (m, 2H), 2.05-1.91 (m, 2H), 1.86-1.78 (m, 2H). Mass (m/z): 408.5 [M+H]⁺.

### Compound 511

### 7-((4-(4-hydroxy-4-(trifluoromethyl)piperidin-1-yl)-2-methylphenyl) amino)-5-methyl-2H-benzo[b][1,4]oxazin-3(4H)-one

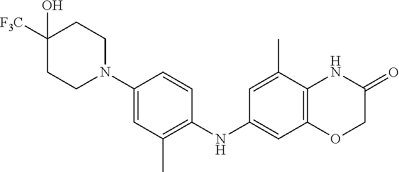 [SMILES: Cc1cc(N2CCC(O)(C(F)(F)F)CC2)ccc1Nc1cc(C)c2c(c1)OCC(=O)N2]

The titled compound 511 (50.4 mg, 30%) as a purple solid was prepared according to the procedure outlined for compound 1. ¹H NMR (400 MHz, DMSO-d₆) δ 9.94 (s, 1H), 7.01 (s, 1H), 6.96 (d, J=8.0 Hz, 1H), 6.85 (s, 1H), 6.76 (d, J=8.8 Hz, 1H), 6.15 (s, 1H), 6.09 (s, 1H), 5.94 (s, 1H), 4.40 (s, 2H), 3.52 (d, J=12.0 Hz, 2H), 2.89 (t, J=12.0 Hz, 2H), 2.10 (s, 6H), 1.88-1.63 (m, 4H). Mass (m/z): 437.5 [M+H]⁺.

### Compound 512

### 6-methyl-7-((2-methyl-4-(4-(trifluoromethyl)piperidin-1-yl)phenyl) amino)-2H-benzo[b][1,4]oxazin-3(4H)-one

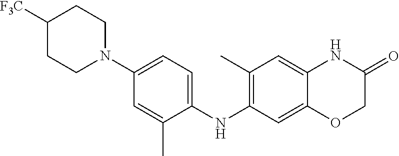 [SMILES: Cc1cc(N2CCC(C(F)(F)F)CC2)ccc1Nc1cc2c(cc1C)NC(=O)CO2]

The titled compound 512 (4.6 mg, 2.8%) as a grey solid was prepared according to the procedure outlined for compound 1. ¹H NMR (400 MHz, CDCl₃) δ 7.01-6.64 (m, 3H), 6.53 (s, 1H), 6.17 (s, 1H), 4.36 (s, 2H), 3.64-3.51 (m, 2H), 3.29-3.21 (m, 1H), 2.71-2.49 (m, 2H), 2.08 (s, 6H), 1.99-1.85 (m, 2H), 1.81-1.62 (m, 2H). Mass (m/z): 420.5 [M+H]⁺.

### Compound 513

### 5-((2-methyl-4-(4-(trifluoromethyl)piperidin-1-yl)phenyl)amino)-1,3-dihydro-2H-benzo[d]imidazol-2-one

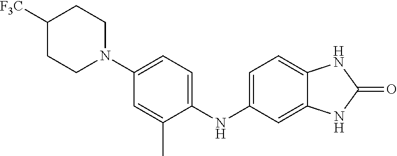 [SMILES: Cc1cc(N2CCC(C(F)(F)F)CC2)ccc1Nc1ccc2[nH]c(=O)[nH]c2c1]

The titled compound 513 (3.6 mg, 0.48%) as a purple solid was prepared according to the procedure outlined for compound 1. ¹H NMR (400 MHz, DMSO-d₆) δ 10.21 (s, 2H), 6.94 (d, J=8.8 Hz, 1H), 6.83 (d, J=3.6 Hz, 2H), 6.75-6.67 (m, 2H), 6.36 (dd, J=8.4, 2.0 Hz, 1H), 6.31 (d, J=2.0 Hz, 1H), 3.65 (d, J=12.0 Hz, 2H), 2.63 (dd, J=12.0, 10.4 Hz, 2H), 2.44 (dd, J=8.8, 3.6 Hz, 1H), 2.12 (s, 3H), 1.88 (d, J=12.4 Hz, 2H), 1.56 (m, J=12.4, 8.4 Hz, 2H). Mass (m/z): 390.8 [M+H]⁺.

### Compound 514

### 3,3-dimethyl-5-((2-methyl-4-(4-(trifluoromethyl)piperidin-1-yl)phenyl) amino)indolin-2-one

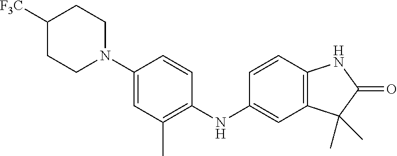 [SMILES: Cc1cc(N2CCC(C(F)(F)F)CC2)ccc1Nc1ccc2c(c1)C(C)(C)C(=O)N2]

The titled compound 514 (4.8 mg, 0.99%) as a purple solid was prepared according to the procedure outlined for compound 1. ¹H NMR (400 MHz, DMSO-d₆) δ 10.03 (s, 1H), 6.92 (d, J=8.8 Hz, 1H), 6.87 (s, 1H), 6.82 (d, J=2.8 Hz, 1H), 6.76-6.69 (m, 2H), 6.64 (d, J=8.0 Hz, 1H), 6.52 (dd, J=8.4, 2.0 Hz, 1H), 3.64 (d, J=12.4 Hz, 2H), 2.62 (dd, J=12.0, 10.0 Hz, 2H), 2.43 (d, J=8.8 Hz, 1H), 2.12 (s, 3H), 1.88 (d, J=12.8 Hz, 2H), 1.61-1.51 (m, 2H), 1.20 (s, 6H). Mass (m/z): 417.8 [M+H]⁺.

### Compound 515

### 7-((2-methyl-4-(4-(trifluoromethyl)piperidin-1-yl)phenyl)amino)-2H-benzo[b][1,4]oxazin-3(4H)-one

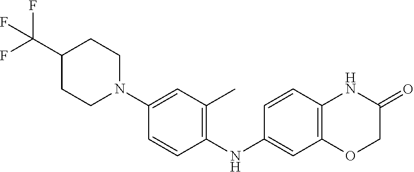 [SMILES: Cc1cc(N2CCC(C(F)(F)F)CC2)ccc1Nc1ccc2c(c1)OCC(=O)N2]

The titled compound 515 (14.4 mg, 9.2%) as a blue solid was prepared according to the procedure outlined for compound 1. ¹H NMR (400 MHz, DMSO-d₆) δ 10.44 (s, 1H), 7.11-6.91 (m, 2H), 6.87-6.64 (m, 3H), 6.36-6.15 (m, 2H), 4.41 (d, J=2.4 Hz, 2H), 3.68 (d, J=12.0 Hz, 2H), 2.64 (s, 2H), 2.42-2.39 (m, 1H), 2.11 (s, 3H), 1.88 (d, J=12.4 Hz, 2H), 1.56 (q, J=12.8 Hz, 2H). Mass (m/z): 406.2 [M+H]⁺.

### Compound 516

### 7-((2-methoxy-4-(4-(trifluoromethyl)piperidin-1-yl)phenyl)amino)-2H-benzo[b][1,4]oxazin-3(4H)-one

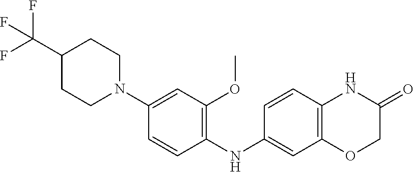 [SMILES: COc1cc(N2CCC(C(F)(F)F)CC2)ccc1Nc1ccc2c(c1)OCC(=O)N2]

The titled compound 516 (26.3 mg, 17%) as a white solid was prepared according to the procedure outlined for compound 1. ¹H NMR (400 MHz, DMSO-d₆) δ 10.39 (s, 1H), 7.05-6.89 (m, 2H), 6.65 (d, J=9.2 Hz, 2H), 6.44 (dd, J=15.2, 6.4 Hz, 3H), 4.45 (d, J=2.4 Hz, 2H), 3.76 (d, J=2.4 Hz, 3H), 3.70 (d, J=12.4 Hz, 2H), 2.67 (d, J=9.2 Hz, 2H), 2.51-2.49 (m, 1H), 1.89 (d, J=12.4 Hz, 2H), 1.58 (q, J=11.6, 11.2 Hz, 2H). Mass (m/z): 424.4 [M+H]⁺.

### Compound 517

### 7-(4-(piperidin-1-yl)phenylamino)-2H-benzo[b][1,4]oxazin-3(4H)-one

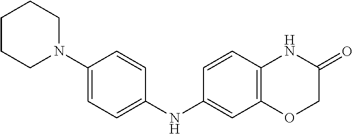 [SMILES: O=C1COc2cc(Nc3ccc(N4CCCCC4)cc3)ccc2N1]

The titled compound 517 (48.5 mg, 22.8%) as a white solid was prepared according to the procedure outlined for compound 1. ¹H NMR (400 MHz, DMSO-d₆) δ 10.43 (s, 1H), 6.93-6.91 (m, 2H), 6.86-6.84 (m, 2H), 6.71 (d, J=8.0 Hz, 1H), 6.53-6.49 (m, 2H), 4.74 (s, 2H), 3.02-2.96 (m, 4H), 1.65-1.58 (m, 4H), 1.53-1.45 (m, 2H). Mass (m/z): 324.1 [M+H]⁺.

### Compound 518

### 7-((4-(4-fluoropiperidin-1-yl)-2-methylphenyl)amino)-2H-benzo[b][1,4]oxazin-3(4H)-one

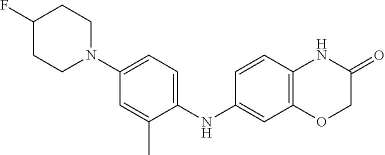 [SMILES: Cc1cc(N2CCC(F)CC2)ccc1Nc1ccc2c(c1)OCC(=O)N2]

The titled compound 518 (55.4 mg, 23.7%) as a yellow solid was prepared according to the procedure outlined for compound 1. ¹H NMR (400 MHz, DMSO-d₆) δ 10.37 (s, 1H), 6.97 (d, J=8.8 Hz, 1H), 6.84 (s, 1H), 6.76 (d, J=8.8 Hz, 1H), 6.66 (d, J=8.4 Hz, 1H), 6.28-6.24 (m, 2H), 4.91-4.71 (m, 1H), 4.44 (s, 2H), 3.31-3.22 (m, 2H), 3.08-2.98 (m, 2H), 2.11 (s, 3H), 2.04-1.89 (m, 2H), 1.85-1.72 (m, 2H). Mass (m/z): 356.1 [M+H]⁺.

### Compound 519

### 7-((4-(3,5-dimethyl-4-(2,2,2-trifluoroethyl)piperazin-1-yl)-2-methylphenyl)amino)-2H-benzo[b][1,4]oxazin-3(4H)-one

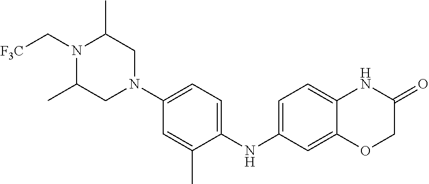 [SMILES: Cc1cc(N2CC(C)N(CC(F)(F)F)C(C)C2)ccc1Nc1ccc2c(c1)OCC(=O)N2]

The titled compound 519 (12.4 mg, 10.5%) as a yellow solid was prepared according to the procedure outlined for compound 1. ¹H NMR (400 MHz, DMSO-d₆) δ 10.37 (s, 1H), 7.07 (s, 1H), 6.95 (d, J=8.8 Hz, 1H), 6.81 (s, 1H), 6.73 (d, J=8.8 Hz, 1H), 6.65 (d, J=8.4 Hz, 1H), 6.31-6.17 (m, 2H), 4.44 (s, 2H), 3.47 (d, J=11.6 Hz, 2H), 3.38 (t, J=10.4 Hz, 2H), 2.82 (t, J=8.4 Hz, 2H), 2.33 (t, J=11.2 Hz, 2H), 2.11 (s, 3H), 1.10 (d, J=6.4 Hz, 6H). Mass (m/z): 449.2 [M+H]⁺.

### Compound 520

### 7-((2-methyl-6-(2,2,6-trimethylmorpholino)pyridin-3-yl)amino)-2H-benzo[b][1,4]oxazin-3(4H)-one

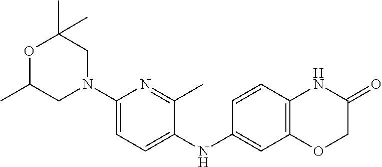 [SMILES: Cc1nc(N2CC(C)OC(C)(C)C2)ccc1Nc1ccc2c(c1)OCC(=O)N2]

The titled compound 520 (12.4 mg, 10.5%) as a grey solid was prepared according to the procedure outlined for compound 1. ¹H NMR (400 MHz, DMSO-d₆) δ 10.36 (s, 1H), 7.27 (d, J=8.8 Hz, 1H), 7.11 (s, 1H), 6.66 (d, J=2.0 Hz, 1H), 6.65-6.62 (m, 1H), 6.20-6.14 (m, 2H), 4.44 (s, 2H), 4.13 (d, J=12.0 Hz, 1H), 3.93 (d, J=12.4 Hz, 1H), 3.89-3.80 (m, 1H), 2.48-2.43 (m, 1H), 2.32-2.23 (m, 1H), 2.20 (s, 3H), 1.21 (s, 3H), 1.18 (s, 3H), 1.09 (d, J=6.0 Hz, 3H). Mass (m/z): 383.1 [M+H]⁺.

### Compound 521

### 7-((2-methyl-6-(4-(2,2,2-trifluoroethyl)piperazin-1-yl)pyridin-3-yl) amino)-2H-benzo[b][1,4]oxazin-3(4H)-one

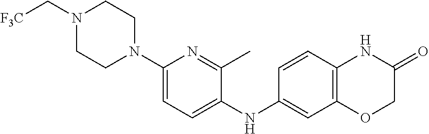 [SMILES: Cc1nc(N2CCN(CC(F)(F)F)CC2)ccc1Nc1ccc2c(c1)OCC(=O)N2]

The titled compound 521 (28.1 mg, 18.3%) as a white solid was prepared according to the procedure outlined for compound 1. ¹H NMR (400 MHz, DMSO-d₆) δ 10.36 (s, 1H), 7.28 (d, J=8.8 Hz, 1H), 7.13 (s, 1H), 6.65 (dd, J=8.4, 5.6 Hz, 2H), 6.24-6.11 (m, 2H), 4.44 (s, 2H), 3.43 (t, J=5.2 Hz, 4H), 3.22 (q, J=10.4 Hz, 2H), 2.70 (t, J=5.2 Hz, 4H), 2.21 (s, 3H). Mass (m/z): 422.3[M+H]⁺.

### Compound 522

### 7-((6-((2S,6R)-2,6-dimethylmorpholino)-2-ethylpyridin-3-yl)amino)-2H-benzo[b][1,4]oxazin-3(4H)-one

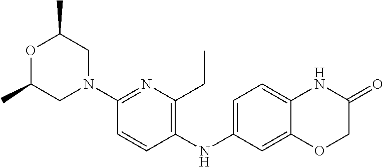 [SMILES: CCc1nc(N2C[C@@H](C)O[C@@H](C)C2)ccc1Nc1ccc2c(c1)OCC(=O)N2]

The titled compound 522 (35.3 mg, 45.6%) as a white solid was prepared according to the procedure outlined for compound 1. ¹H NMR (400 MHz, DMSO-d₆) δ 10.36 (s, 1H), 7.28 (dd, J=8.8, 2.4 Hz, 1H), 7.10 (s, 1H), 6.70-6.57 (m, 2H), 6.21-6.09 (m, 2H), 4.49-4.40 (m, 2H), 4.08 (d, J=12.4 Hz, 2H), 3.68-3.51 (m, 2H), 2.61-2.53 (m, 2H), 2.33 (t, J=11.6 Hz, 2H), 1.20-1.07 (m, 9H). Mass (m/z): 383.3 [M+H]⁺.

### Compound 523

### 7-((4-(4-(trifluoromethyl)piperidin-1-yl)phenyl)amino)-2H-benzo[b][1,4]oxazin-3(4H)-one

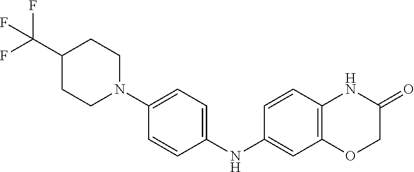 [SMILES: O=C1COc2cc(Nc3ccc(N4CCC(C(F)(F)F)CC4)cc3)ccc2N1]

The titled compound 523 (29 mg, 18.1%) as a yellow solid was prepared according to the procedure outlined for compound 1. ¹H NMR (400 MHz, DMSO-d₆) δ 10.51 (s, 1H), 7.66 (s, 1H), 7.00-6.84 (m, 4H), 6.77 (d, J=8.8 Hz, 1H), 6.58 (d, J=3.6 Hz, 1H), 6.48 (d, J=8.8 Hz, 1H), 4.45 (d, J=2.4 Hz, 2H), 3.60 (d, J=12.0 Hz, 2H), 2.61 (s, 3H), 1.88 (d, J=12.4 Hz, 2H), 1.57 (q, J=12.8 Hz, 2H). Mass (m/z): 392.2 [M+H]⁺.

### Compound 524

### 7-((4-(4,4-difluoropiperidin-1-yl)phenyl)amino)-2H-benzo[b][1,4]oxazin-3(4H)-one

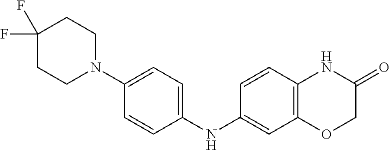 [SMILES: O=C1COc2cc(Nc3ccc(N4CCC(F)(F)CC4)cc3)ccc2N1]

The titled compound 524 (11.7 mg, 6.9%) as a pink solid was prepared according to the procedure outlined for compound 1. ¹H NMR (400 MHz, DMSO-d₆) δ 10.45 (s, 1H), 7.73 (s, 1H), 6.93 (s, 3H), 6.71 (d, J=8.4 Hz, 1H), 6.54 (d, J=16.0 Hz, 2H), 4.48 (d, J=2.4 Hz, 2H), 3.18 (d, J=6.0 Hz, 4H), 2.06 (p, J=8.8 Hz, 4H). Mass (m/z): 360.1 [M+H]⁺.

### Compound 525

### 7-((4-(4,4-difluoropiperidin-1-yl)-2-methylphenyl)amino)-2H-benzo[b][1,4]oxazin-3(4H)-one

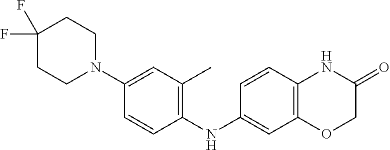 [SMILES: Cc1cc(N2CCC(F)(F)CC2)ccc1Nc1ccc2c(c1)OCC(=O)N2]

The titled compound 525 (14.4 mg, 8.7%) as pink oil was prepared according to the procedure outlined for compound 1. ¹H NMR (400 MHz, DMSO-d₆) δ 10.38 (s, 1H), 7.09 (s, 1H), 6.97 (dd, J=8.8, 2.4 Hz, 1H), 6.88 (s, 1H), 6.78 (d, J=8.8 Hz, 1H), 6.66 (dd, J=8.4, 2.4 Hz, 1H), 6.36-6.19 (m, 2H), 4.45 (d, J=2.4 Hz, 2H), 3.26-3.24 (m, 4H), 2.11 (d, J=2.4 Hz, 3H), 2.04 (dq, J=12.8, 6.8, 5.2 Hz, 4H). Mass (m/z): 374.2 [M+H]⁺

### Compound 526

### N5-(2-methyl-4-(4-(trifluoromethyl)piperidin-1-yl)phenyl)-1H-benzo[d]imidazole-2,5-diamine

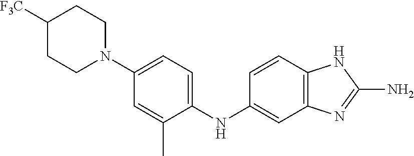 [SMILES: Cc1cc(N2CCC(C(F)(F)F)CC2)ccc1Nc1ccc2[nH]c(N)nc2c1]

The titled compound 526 (21.1 mg 91.8%) as a black solid was prepared according to the procedure outlined for compound 1. ¹H NMR (400 MHz, DMSO-d₆) δ 10.52 (s, 1H), 6.91-6.89 (m, 2H), 6.80 (d, J=2.4, 1H), 6.69-6.67 (m, 1H), 6.58 (d, J=1.6, 2H), 6.44 (s, 1H), 5.92 (s, 2H), 3.60 (d, J=12.0, 2H), 2.60 (t, J=12.0, 2H), 2.42-2.37 (m, 1H), 2.15 (d, J=4.4, 3H), 1.87 (d, J=12.0, 2H), 1.62-1.51 (m, 2H). Mass (m/z): 389.7 [M+H]⁺.

### Compound 527

### 3,3-dimethyl-6-((2-methyl-4-(4-(trifluoromethyl)piperidin-1-yl) phenyl)amino)indolin-2-one

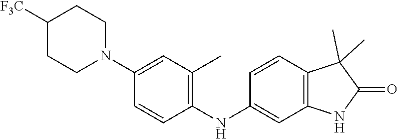 [SMILES: Cc1cc(N2CCC(C(F)(F)F)CC2)ccc1Nc1ccc2c(c1)NC(=O)C2(C)C]

The titled compound 527 (15.3 mg, 4.73%) as a purple solid was prepared according to the procedure outlined for compound 1. ¹H NMR (400 MHz, DMSO-d₆) δ 10.03 (s, 1H), 7.19 (s, 1H), 6.98 (dd, J=23.6, 8.4 Hz, 2H), 6.86 (d, J=2.8 Hz, 1H), 6.77 (dd, J=8.8, 2.8 Hz, 1H), 6.24 (dd, J=8.0, 2.0 Hz, 1H), 6.16 (d, J=2.0 Hz, 1H), 3.70 (d, J=12.4 Hz, 2H), 2.65 (dd, J=12.0, 10.4 Hz, 2H), 2.47-2.40 (m, 1H), 2.13 (s, 3H), 1.89 (d, J=12.0 Hz, 2H), 1.56 (m, J=12.4, 8.8 Hz, 2H), 1.18 (s, 6H). Mass (m/z): 417.7 [M+H]⁺.

### Compound 528

### N-(4-(2,6-dimethylmorpholino)-2-methylphenyl)-1,2,3,4-tetrahydroisoquinolin-6-amine

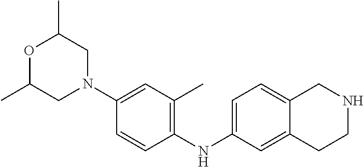 [SMILES: Cc1cc(N2CC(C)OC(C)C2)ccc1Nc1ccc2c(c1)CCNC2]

The titled compound 528 (25.1 mg, 15.7%) as a yellow solid was prepared according to the procedure outlined for compound 1. ¹H NMR (400 MHz, DMSO-d₆) δ 7.00-6.91 (m, 2H), 6.81 (s, 1H), 6.72 (d, J=8.4 Hz, 2H), 6.43 (d, J=8.4 Hz, 1H), 6.33 (s, 1H), 3.72-3.62 (m, 4H), 3.48 (d, J=11.6 Hz, 2H), 3.33-3.32 (m, 2H), 2.86 (s, 2H), 2.19 (t, J=11.2 Hz, 2H), 2.11 (s, 3H), 1.17-1.11 (m, 6H). Mass (m/z): 352.5 [M+H]⁺.

### Compound 529

### N-(2-methyl-4-(2-methylmorpholino)phenyl)-1,2,3,4-tetrahydroisoquinolin-6-amine

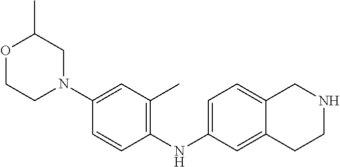 [SMILES: Cc1cc(N2CCOC(C)C2)ccc1Nc1ccc2c(c1)CCNC2]

The titled compound 529 (15 mg, 9.7%) as a yellow solid was prepared according to the procedure outlined for compound 1. ¹H NMR (400 MHz, DMSO-d₆) δ 6.99-6.92 (m, 2H), 6.81 (s, 1H), 6.73 (d, J=8.4 Hz, 2H), 6.44 (d, J=8.4 Hz, 1H), 6.34 (s, 1H), 3.89 (d, J=11.2 Hz, 1H), 3.70 (s, 2H), 3.68-3.56 (m, 2H), 3.49 (d, J=11.6 Hz, 1H), 3.39 (d, J=12.0 Hz, 1H), 2.90-2.83 (m, 2H), 2.70-2.58 (m, 2H), 2.35-2.23 (m, 2H), 2.11 (s, 3H), 1.14 (d, J=6.4 Hz, 3H). Mass (m/z): 338.5 [M+H]⁺.

### Compound 530

### 8-((4-(4-(trifluoromethyl)piperidin-1-yl)phenyl)amino)-2,3-dihydrobenzo[b][1,4]oxazepin-4(5H)-one

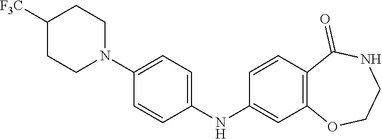 [SMILES: O=C1NCCOc2cc(Nc3ccc(N4CCC(C(F)(F)F)CC4)cc3)ccc21]

The titled compound 530 (26.7 mg, 17%) as an off-white solid was prepared according to the procedure outlined for compound 1. ¹H NMR (400 MHz, DMSO-d₆) δ 8.25 (s, 1H), 7.96-7.89 (m, 1H), 7.67 (d, J=8.8 Hz, 1H), 7.07-7.00 (m, 2H), 6.98-6.91 (m, 2H), 6.57 (d, J=8.8 Hz, 1H), 6.35 (s, 1H), 4.21 (d, J=4.8 Hz, 2H), 3.69 (d, J=12.0 Hz, 2H), 3.33 (d, J=2.0 Hz, 2H), 2.66 (t, J=12.0 Hz, 2H), 2.47-2.36 (m, 1H), 1.88 (d, J=12.4 Hz, 2H), 1.64-1.49 (m, 2H). Mass (m/z): 406.5 [M+H]⁺.

### Compound 531

### 8-((2-methyl-4-(4-(trifluoromethyl)piperidin-1-yl)phenyl)amino)-3,4-dihydrobenzo[f][1,4]oxazepin-5(2H)-one

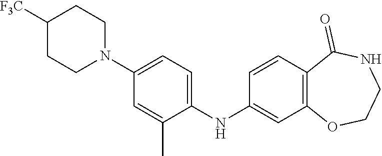 [SMILES: Cc1cc(N2CCC(C(F)(F)F)CC2)ccc1Nc1ccc2c(c1)OCCNC2=O]

The titled compound 531 (18 mg, 10%) as a grey solid was prepared according to the procedure outlined for compound 1. ¹H NMR (400 MHz, DMSO-d₆) δ 7.86 (s, 1H), 7.76 (s, 1H), 7.63 (d, J=8.8 Hz, 1H), 7.01 (d, J=8.4 Hz, 1H), 6.88 (s, 1H), 6.80 (d, J=8.8 Hz, 1H), 6.34 (d, J=8.8 Hz, 1H), 6.00 (s, 1H), 4.18 (d, J=5.2 Hz, 2H), 3.75 (d, J=12.0 Hz, 2H), 3.28 (s, 2H), 2.73-2.62 (m, 2H), 2.41-2.37 (m, 1H), 2.11 (s, 3H), 1.88 (d, J=12.4 Hz, 2H), 1.63-1.49 (m, 2H). Mass (m/z): 420.5 [M+H]⁺.

### Compound 532

### 4-methyl-7-(methyl(4-(4-(trifluoromethyl)piperidin-1-yl)phenyl)amino)-2H-benzo[b][1,4]oxazin-3(4H)-one

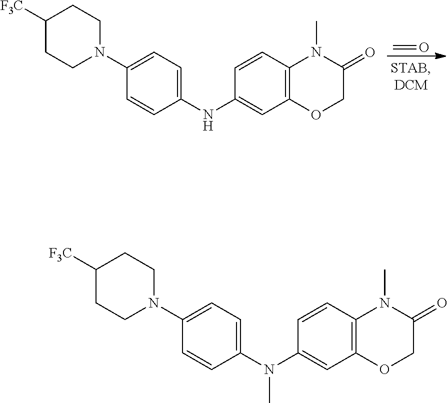

To a solution of 4-methyl-7-((4-(4-(trifluoromethyl)piperidin-1-yl)phenyl) amino)-2H-benzo[b][1,4]oxazin-3(4H)-one (100 mg, 0.24 mmol) in DCM (2 mL) was added formaldehyde (15 mg, 0.49 mmol) and sodium triacetoxyhydroborate (104 mg, 0.49 mmol). The mixture was stirred for 2 hs and concentrated under vacuum. The residue was purified by C18 column to give 4-methyl-7-(methyl(4-(4-(trifluoromethyl)piperidin-1-yl) phenyl)amino)-2H-benzo[b][1,4]oxazin-3(4H)-one (22.5 mg, 21.7%) as an off-white solid. ¹H NMR (400 MHz, DMSO-d₆) δ 7.03-6.92 (m, 5H), 6.43 (d, J=8.8 Hz, 1H), 6.37 (s, 1H), 4.56 (s, 2H), 3.73 (d, J=12.4 Hz, 2H), 3.22 (s, 3H), 3.15 (s, 3H), 2.74-2.63 (m, 2H), 2.45-2.40 (m, 1H), 1.89 (d, J=12.8 Hz, 2H), 1.63-1.49 (m, 2H). Mass (m/z): 420.5 [M+H]⁺.

### Compound 533

### 5-(4-methoxybenzyl)-8-((2-methyl-4-(4-(trifluoromethyl)piperidin-1-yl) phenyl)amino)-2,3-dihydrobenzo[b][1,4]oxazepin-4(5H)-one

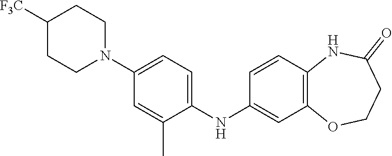 [SMILES: Cc1cc(N2CCC(C(F)(F)F)CC2)ccc1Nc1ccc2c(c1)OCCC(=O)N2]

The titled compound 533 (7.5 mg, 19%) as a grey solid was prepared according to the procedure outlined for compound 1. ¹H NMR (400 MHz, DMSO-d₆) δ 9.34 (s, 1H), 7.17 (s, 1H), 6.98 (d, J=9.2 Hz, 1H), 6.85 (s, 1H), 6.82-6.72 (m, 2H), 6.36 (d, J=8.8 Hz, 1H), 6.26 (s, 1H), 4.35-4.28 (m, 2H), 3.69 (d, J=12.4 Hz, 2H), 2.67-2.63 (m, 2H), 2.44-2.37 (m, 1H), 2.11 (s, 3H), 1.88 (d, J=12.8 Hz, 2H), 1.63-1.49 (m, 2H). Mass (m/z): 420.5 [M+H]⁺.

### Compound 534

### 8-((4-(4-(trifluoromethyl)piperidin-1-yl)phenyl)amino)-2,3-dihydrobenzo[b][1,4]oxazepin-4(5H)-one

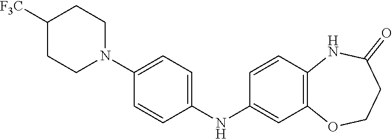 [SMILES: O=C1CCOc2cc(Nc3ccc(N4CCC(C(F)(F)F)CC4)cc3)ccc2N1]

The titled compound 534 (8.8 mg, 22%) as a grey solid was prepared according to the procedure outlined for compound 1. ¹H NMR (400 MHz, DMSO-d₆) δ 9.41 (s, 1H), 7.78 (s, 1H), 6.96 (d, J=8.4 Hz, 2H), 6.90 (d, J=8.4 Hz, 2H), 6.84 (d, J=8.6 Hz, 1H), 6.60 (d, J=8.7 Hz, 1H), 6.55 (s, 1H), 4.34 (t, J=6.2 Hz, 2H), 3.62 (d, J=12.1 Hz, 2H), 2.68-2.58 (m, 4H), 2.43-2.36 (m, 1H), 1.88 (d, J=12.7 Hz, 2H), 1.66-1.49 (m, 2H). Mass (m/z): 406.5 [M+H]⁺.

### Compound 535

### 6-((2-methyl-4-(4-(trifluoromethyl)piperidin-1-yl)phenyl)amino)-3,4-dihydroquinazolin-2(1H)-one

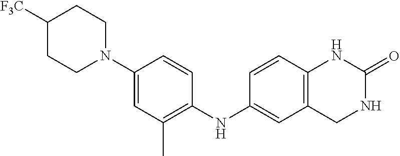 [SMILES: Cc1cc(N2CCC(C(F)(F)F)CC2)ccc1Nc1ccc2c(c1)CNC(=O)N2]

The titled compound 535 (22.0 mg, 15.28%) as a white solid was prepared according to the procedure outlined for compound 1. ¹H NMR (400 MHz, DMSO-d₆) δ 8.70 (s, 1H), 6.92 (d, J=8.8 Hz, 1H), 6.86-6.77 (m, 2H), 6.71 (dd, J=8.8, 2.8 Hz, 1H), 6.58 (d, J=8.4 Hz, 2H), 6.52 (dd, J=8.4, 2.4 Hz, 1H), 6.46 (d, J=2.0 Hz, 1H), 4.19 (s, 2H), 3.64 (d, J=12.4 Hz, 2H), 2.68-2.57 (m, 2H), 2.45-2.36 (m, 1H), 2.11 (s, 3H), 1.87 (d, J=12.0 Hz, 2H), 1.61-1.50 (m, 2H). Mass (m/z): 404.8 [M+H]⁺.

### Compound 536

### 5-N-[4-(2,6-dimethylmorpholin-4-yl)-2-methylphenyl]-1-methyl-1,3-benzodiazole-2,5-diamine

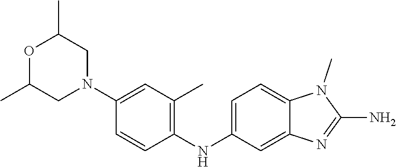 [SMILES: Cc1cc(N2CC(C)OC(C)C2)ccc1Nc1ccc2c(c1)nc(N)n2C]

The titled compound 536 (2.6 mg, 0.66%) as a yellow solid was prepared according to the procedure outlined for compound 1. ¹H NMR (400 MHz, DMSO-d₆) δ 6.91 (d, J=8.4 Hz, 2H), 6.79 (d, J=2.4 Hz, 1H), 6.71-6.60 (m, 2H), 6.56 (d, J=1.2 Hz, 1H), 6.44 (dd, J=8.4, 1.6 Hz, 1H), 6.36 (s, 2H), 3.73-3.63 (m, 2H), 3.43 (d, J=10.0 Hz, 5H), 2.22-2.10 (m, 5H), 1.14 (d, J=6.4 Hz, 6H). Mass (m/z): 366.0 [M+H]⁺.

### Compound 537

### 2-methyl-3-((3-oxo-3,4-dihydro-2H-benzo[b][1,4]oxazin-7-yl)amino)-6-(4-(trifluoromethyl)piperidin-1-yl)benzonitrile

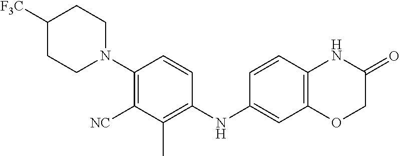 [SMILES: Cc1c(Nc2ccc3c(c2)OCC(=O)N3)ccc(N2CCC(C(F)(F)F)CC2)c1C#N]

The titled compound 537 (29.3 mg, 17.03%) as a white solid was prepared according to the procedure outlined for compound 1. ¹H NMR (400 MHz, CDCl₃) δ 8.17 (s, 1H), 6.90 (d, J=8.8 Hz, 1H), 6.68 (d, J=8.4 Hz, 1H), 6.42 (dd, J=11.6, 3.2 Hz, 2H), 4.57 (s, 2H), 3.54 (d, J=12.0 Hz, 2H), 2.78 (t, J=11.2 Hz, 2H), 2.41 (s, 3H), 2.15 (ddd, J=12.0, 8.0, 4.0 Hz, 1H), 2.04-1.86 (m, 4H). Mass (m/z): 430.8 [M+H]⁺.

### Compound 538

### 4-methyl-7-((3-methyl-4-(4-(2,2,2-trifluoroethyl)piperazin-1-yl)phenyl) amino)-2H-benzo[b][1,4]oxazin-3(4H)-one

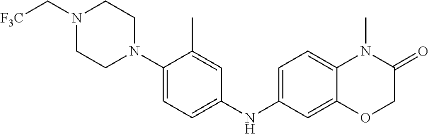 [SMILES: Cc1cc(Nc2ccc3c(c2)OCC(=O)N3C)ccc1N1CCN(CC(F)(F)F)CC1]

The titled compound 538 (10.0 mg, 12.34%) as a pink solid was prepared according to the procedure outlined for compound 1. ¹H NMR (400 MHz, CDCl₃) δ 7.17 (s, 1H), 6.94 (d, J=8.8 Hz, 1H), 6.87 (d, J=8.8 Hz, 1H), 6.80 (d, J=2.8 Hz, 1H), 6.70 (dd, J=8.8, 2.8 Hz, 1H), 6.31 (dd, J=8.8, 2.4 Hz, 1H), 6.23 (d, J=2.4 Hz, 1H), 4.49 (s, 2H), 3.25-3.18 (m, 2H), 3.16 (s, 3H), 3.08-3.01 (m, 4H), 2.75-2.68 (m, 4H), 2.08 (s, 3H). Mass (m/z): 434.9 [M+H]⁺.

### Compound 539

### 6-((2-methyl-4-(4-(trifluoromethyl)piperidin-1-yl)phenyl)amino)-1,4-dihydro-2H-benzo[d][1,3]oxazin-2-one

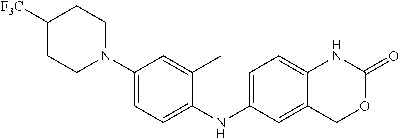 [SMILES: Cc1cc(N2CCC(C(F)(F)F)CC2)ccc1Nc1ccc2c(c1)COC(=O)N2]

The titled compound 539 (5 mg, 3.2%) as a white solid was prepared according to the procedure outlined for compound 1. ¹H NMR (400 MHz, DMSO-d₆) δ 9.78 (s, 1H), 7.03 (s, 1H), 6.91 (d, J=8.8 Hz, 1H), 6.80 (d, J=2.8 Hz, 1H), 6.70 (dd, J=8.8, 2.8 Hz, 1H), 6.64 (d, J=8.8 Hz, 1H), 6.56 (dd, J=8.8, 2.4 Hz, 1H), 6.46 (d, J=2.4 Hz, 1H), 5.10 (s, 2H), 3.64 (d, J=12.4 Hz, 2H), 2.60 (td, J=12.4, 2.0 Hz, 2H), 2.08 (s, 3H), 1.84 (d, J=11.6 Hz, 2H), 1.52 (dd, J=12.4, 3.6 Hz, 2H), 1.20 (s, 1H). Mass (m/z): 405.9 [M+H]⁺.

### Compound 540

### 7-((4-(4-hydroxy-4-methylpiperidin-1-yl)phenyl)amino)-2H-benzo[b][1,4]oxazin-3(4H)-one

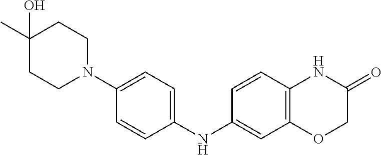 [SMILES: CC1(O)CCN(c2ccc(Nc3ccc4c(c3)OCC(=O)N4)cc2)CC1]

The titled compound 540 (46.8 mg, 30.2%) as a grey solid was prepared according to the procedure outlined for compound 1. ¹H NMR (400 MHz, DMSO-d₆) δ 10.43 (s, 1H), 7.66 (s, 1H), 7.11-6.31 (m, 7H), 4.47 (d, J=2.4 Hz, 2H), 4.26 (s, 1H), 3.28-2.75 (m, 4H), 1.57 (s, 4H), 1.15 (s, 3H). Mass (m/z): 354.1 [M+H]⁺.

### Compound 541

### 7-((4-(4-hydroxy-4-methylpiperidin-1-yl)-2-methylphenyl)amino)-2H-benzo[b][1,4]oxazin-3(4H)-on

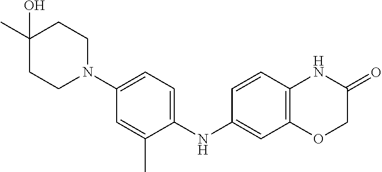 [SMILES: Cc1cc(N2CCC(C)(O)CC2)ccc1Nc1ccc2c(c1)OCC(=O)N2]

The titled compound 541 (17.6 mg, 10.9%) as a grey solid was prepared according to the procedure outlined for compound 1. ¹H NMR (400 MHz, DMSO-d₆) δ 10.37 (s, 1H), 7.26-6.46 (m, 5H), 6.43-5.99 (m, 2H), 4.44 (s, 2H), 4.26 (s, 1H), 3.12 (m, 4H), 2.10 (s, 3H), 1.56 (s, 4H), 1.15 (s, 3H). Mass (m/z): 368.1 [M+H]⁺.

### Compound 542

### 7-((4-(4-(2-hydroxypropan-2-yl)piperidin-1-yl)-2-methylphenyl)amino)-2H-benzo[b][1,4]oxazin-3(4H)-one

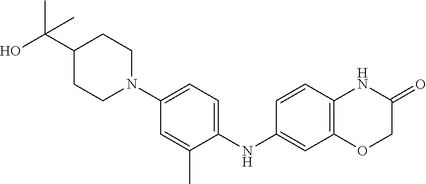 [SMILES: Cc1cc(N2CCC(C(C)(C)O)CC2)ccc1Nc1ccc2c(c1)OCC(=O)N2]

The titled compound 542 (23.8 mg, 15.0%) as a grey solid was prepared according to the procedure outlined for compound 1. ¹H NMR (400 MHz, DMSO-d₆) δ 10.37 (s, 1H), 7.06 (s, 1H), 6.94 (d, J=8.4 Hz, 1H), 6.81 (s, 1H), 6.72 (d, J=8.8 Hz, 1H), 6.65 (d, J=8.4 Hz, 1H), 6.29-6.20 (m, 2H), 4.44 (s, 2H), 4.13 (s, 1H), 3.66 (d, J=11.6 Hz, 2H), 2.53 (m, 2H), 2.10 (s, 3H), 1.77 (d, J=11.2 Hz, 2H), 1.44-1.21 (m, 3H), 1.06 (s, 6H). Mass (m/z): 396.2 [M+H]⁺.

### Compound 543

### 7-((4-(4-(2-hydroxypropan-2-yl)piperidin-1-yl)phenyl)amino)-2H-benzo[b][1,4]oxazin-3(4H)-on

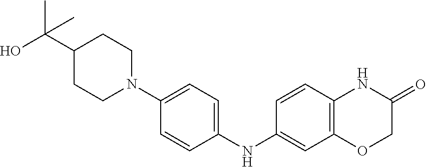 [SMILES: CC(C)(O)C1CCN(c2ccc(Nc3ccc4c(c3)OCC(=O)N4)cc2)CC1]

The titled compound 543 (43.5 mg, 26.8%) as a grey solid was prepared according to the procedure outlined for compound 1. ¹H NMR (400 MHz, DMSO-d₆) δ 10.45 (s, 1H), 7.92-7.43 (m, 1H), 7.18-6.18 (m, 7H), 4.48 (s, 2H), 4.13 (s, 1H), 3.59 (s, 2H), 2.45-2.22 (m, 2H), 1.95-1.56 (m, 2H), 1.43-1.22 (m, 3H), 1.06 (s, 6H). Mass (m/z): 382.2 [M+H]⁺.

### Compound 544

### N-(2-methyl-4-(4-(trifluoromethyl)piperidin-1-yl)phenyl)isoindolin-5-amine

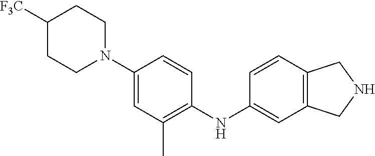 [SMILES: Cc1cc(N2CCC(C(F)(F)F)CC2)ccc1Nc1ccc2c(c1)CNC2]

The titled compound 544 (14.2 mg, 13.2%) as a yellow solid was prepared according to the procedure outlined for compound 1. ¹H NMR (400 MHz, DMSO-d₆) δ 6.97 (t, J=8.0 Hz, 2H), 6.84 (s, 1H), 6.75 (d, J=9.2 Hz, 1H), 6.51 (d, J=6.4 Hz, 2H), 3.92 (d, J=6.0 Hz, 3H), 3.68 (d, J=12.4 Hz, 2H), 2.66 (q, J=12.4 Hz, 3H), 2.44 (d, J=10.0 Hz, 1H), 2.12 (s, 3H), 1.88 (d, J=12.4 Hz, 2H), 1.56 (q, J=12.4 Hz, 2H). Mass (m/z): 376.3 [M+H]⁺.

### Compound 545

### 5-((2-methyl-4-(4-(trifluoromethyl)piperidin-1-yl)phenyl)amino) isoindoline-2-carboxamide

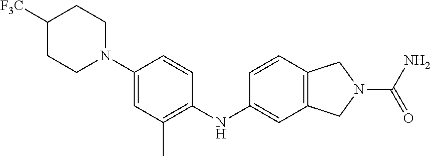 [SMILES: Cc1cc(N2CCC(C(F)(F)F)CC2)ccc1Nc1ccc2c(c1)CN(C(N)=O)C2]

The titled compound 545 (22.1 mg, 18.7%) as a white solid was prepared according to the procedure outlined for compound 1. ¹H NMR (400 MHz, DMSO-d₆) δ 7.24 (s, 1H), 7.01 (t, J=8.0 Hz, 2H), 6.86 (s, 1H), 6.77 (d, J=8.8 Hz, 1H), 6.59 (d, J=8.4 Hz, 1H), 6.52 (s, 1H), 5.87 (s, 2H), 4.42 (s, 4H), 3.70 (d, J=12.4 Hz, 2H), 2.66 (t, J=12.0 Hz, 2H), 2.44 (s, 1H), 2.12 (s, 3H), 1.88 (d, J=12.8 Hz, 2H), 1.56 (q, J=12.4 Hz, 2H). Mass (m/z): 419.3 [M+H]⁺.

### Compound 546

### 7-((5-methoxy-2-methyl-4-(4-(trifluoromethyl)piperidin-1-yl)phenyl) amino)-2H-benzo[b][1,4]oxazin-3(4H)-one

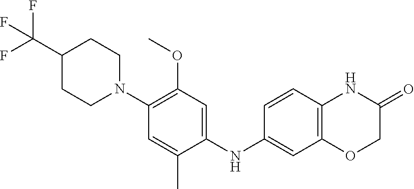 [SMILES: COc1cc(Nc2ccc3c(c2)OCC(=O)N3)c(C)cc1N1CCC(C(F)(F)F)CC1]

The titled compound 546 (25.6 mg, 21.2%) as a pink solid was prepared according to the procedure outlined for compound 1. ¹H NMR (400 MHz, DMSO-d₆) δ 10.41 (s, 1H), 7.18 (s, 1H), 6.70 (d, J=23.2 Hz, 3H), 6.42-6.29 (m, 2H), 4.46 (d, J=2.4 Hz, 2H), 3.68 (d, J=2.4 Hz, 3H), 3.38 (s, 2H), 2.60-2.55 (m, 3H), 2.06 (d, J=2.4 Hz, 3H), 1.87 (d, J=12.4 Hz, 2H), 1.61 (dd, J=14.0, 10.4 Hz, 2H). Mass (m/z): 436.2 [M+H]⁺.

### Compound 547

### 7-((5-methoxy-2-methyl-4-(4-(trifluoromethyl)piperidin-1-yl)phenyl) amino)-2H-pyrido[3,2-b][1,4]oxazin-3(4H)-one

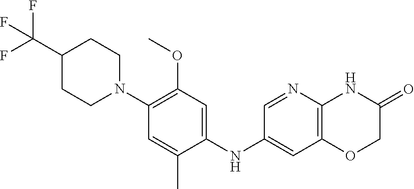 [SMILES: COc1cc(Nc2cnc3c(c2)OCC(=O)N3)c(C)cc1N1CCC(C(F)(F)F)CC1]

The titled compound 547 (18.3 mg, 15.1%) as a pink solid was prepared according to the procedure outlined for compound 1. ¹H NMR (400 MHz, DMSO-d₆) δ 10.91 (s, 1H), 7.45 (s, 1H), 7.34 (s, 1H), 6.75 (s, 1H), 6.72-6.60 (m, 2H), 4.55 (d, J=2.4 Hz, 2H), 3.69 (d, J=2.4 Hz, 3H), 3.37 (d, J=13.6 Hz, 2H), 2.58-2.55 (m, 2H), 2.43-2.39 (m, 1H), 2.08 (d, J=2.4 Hz, 3H), 1.87 (d, J=12.4 Hz, 2H), 1.59 (q, J=12.4 Hz, 2H). Mass (m/z): 437.2 [M+H]⁺.

### Compound 548

### 7-((5-methoxy-2-methyl-4-(4-(trifluoromethyl)piperidin-1-yl)phenyl) amino)-5-methyl-2H-benzo[b][1,4]oxazin-3(4H)-one

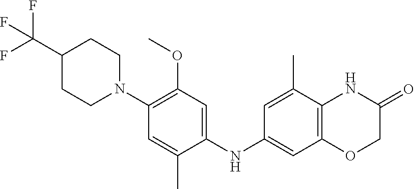 [SMILES: COc1cc(Nc2cc(C)c3c(c2)OCC(=O)N3)c(C)cc1N1CCC(C(F)(F)F)CC1]

The titled compound 548 (24 mg, 19.2%) as a pink solid was prepared according to the procedure outlined for compound 1. ¹H NMR (400 MHz, DMSO-d₆) δ 9.97 (s, 1H), 7.13 (s, 1H), 6.70 (d, J=23.6 Hz, 2H), 6.20 (d, J=24.4 Hz, 2H), 4.41 (s, 2H), 3.68 (d, J=2.4 Hz, 3H), 3.37 (d, J=10.0 Hz, 2H), 2.58-2.55 (m, 2H), 2.43-2.39 (m, 1H), 2.12 (s, 3H), 2.05 (s, 3H), 1.87 (d, J=12.4 Hz, 2H), 1.59 (q, J=12.4 Hz, 2H). Mass (m/z): 450.2 [M+H]⁺.

### Compound 549

### 7-((4-(7-azabicyclo[2.2.1]heptan-7-yl)phenyl)amino)-2H-benzo[b][1,4]oxazin-3(4H)-one

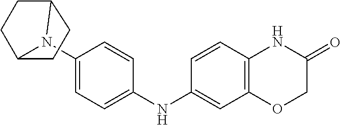 [SMILES: O=C1COc2cc(Nc3ccc(N4C5CCC4CC5)cc3)ccc2N1]

The titled compound 549 (6.8 mg, 4.8%) as a pink solid was prepared according to the procedure outlined for compound 1. ¹H NMR (400 MHz, DMSO-d₆) δ 10.42 (s, 1H), 7.59 (s, 1H), 6.95-6.77 (m, 4H), 6.69 (d, J=8.4 Hz, 1H), 6.55-6.40 (m, 2H), 4.47 (d, J=2.8 Hz, 2H), 4.13 (s, 2H), 1.64 (d, J=7.6 Hz, 4H), 1.37 (d, J=7.6 Hz, 4H). Mass (m/z): 336.2 [M+H]⁺.

### Compound 550

### 6-((4-(4-hydroxy-4-(trifluoromethyl)piperidin-1-yl)phenyl)amino)-2H-benzo[b][1,4]oxazin-3(4H)-one

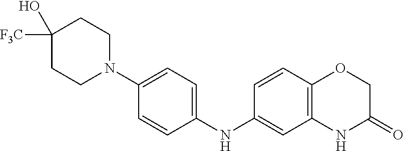 [SMILES: O=C1COc2ccc(Nc3ccc(N4CCC(O)(C(F)(F)F)CC4)cc3)cc2N1]

The titled compound 550 (17.3 mg, 19.4%) as a pink solid was prepared according to the procedure outlined for compound 1. ¹H NMR (400 MHz, DMSO-d₆) δ 10.44 (s, 1H), 7.03 (s, 1H), 7.00-6.90 (m, 1H), 6.85 (s, 1H), 6.80-6.65 (m, 2H), 6.34-6.18 (m, 2H), 5.94 (s, 1H), 4.41 (d, J=2.4 Hz, 2H), 3.51 (d, J=12.0 Hz, 2H), 2.88 (t, J=12.0 Hz, 2H), 2.11 (d, J=2.4 Hz, 3H), 1.89-1.63 (m, 4H). Mass (m/z): 408.2 [M+H]⁺.

### Compound 551

### 1-methyl-6-((2-methyl-4-(4-(trifluoromethyl)piperidin-1-yl)phenyl) amino)-1H-indole-3-carboxamide

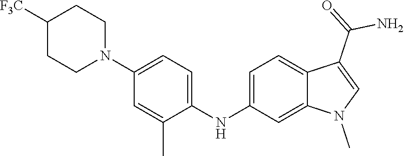 [SMILES: Cc1cc(N2CCC(C(F)(F)F)CC2)ccc1Nc1ccc2c(C(N)=O)cn(C)c2c1]

The titled compound 551 (18 mg, 10.8%) as a black solid was prepared according to the procedure outlined for compound 1. ¹H NMR (400 MHz, DMSO-d₆) δ 7.86 (d, J=8.8 Hz, 1H), 7.71 (s, 1H), 7.10-7.05 (m, 2H), 6.86 (d, J=2.8 Hz, 1H), 6.77-6.74 (m, 1H), 6.71-6.68 (m, 1H), 6.60 (d, J=1.6 Hz, 1H), 3.69 (d, J=12.4 Hz, 2H), 3.61 (s, 3H), 3.31 (m, 1H), 2.63 (d, J=10.4 Hz, 2H), 2.16 (s, 3H), 1.89 (d, J=12.4 Hz, 2H), 1.57 (dd, J=12.4, 4.0 Hz, 2H). Mass (m/z): 430.8 [M+H]⁺.

### Compound 552

### (S)-7-((2-methyl-4-(3-methyl-4-(2,2,2-trifluoroacetyl)piperazin-1-yl) phenyl)amino)-2H-benzo[b][1,4]oxazin-3(4H)-one

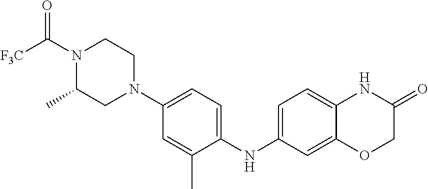 [SMILES: Cc1cc(N2CCN(C(=O)C(F)(F)F)[C@@H](C)C2)ccc1Nc1ccc2c(c1)OCC(=O)N2]

The titled compound 552 (8 mg, 5%) as a pink solid was prepared according to the procedure outlined for compound 1. ¹H NMR (400 MHz, DMSO-d₆) δ 10.38 (s, 1H), 7.11 (s, 1H), 6.99 (d, J=8.8 Hz, 1H), 6.84 (s, 1H), 6.78-6.71 (m, 1H), 6.66 (d, J=8.4 Hz, 1H), 6.32-6.24 (m, 2H), 4.63-4.58 (m, 1H), 4.45 (s, 2H), 4.28-4.20 (m, 1H), 3.79 (d, J=13.6 Hz, 1H), 3.68-3.53 (m, 2H), 3.53-3.47 (m, 1H), 2.93-2.77 (m, 1H), 2.12 (s, 3H), 1.49-1.30 (m, 3H). Mass (m/z): 449.5 [M+H]⁺.

### Compound 553

### (S)-7-((4-(3-methyl-4-(2,2,2-trifluoroacetyl)piperazin-1-yl)phenyl) amino)-2H-benzo[b][1,4]oxazin-3(4H)-one

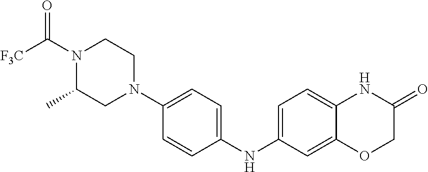 [SMILES: C[C@H]1CN(c2ccc(Nc3ccc4c(c3)OCC(=O)N4)cc2)CCN1C(=O)C(F)(F)F]

The titled compound 553 (24.4 mg, 16.9%) as a grey solid was prepared according to the procedure outlined for compound 1. ¹H NMR (400 MHz, DMSO-d₆) δ 10.45 (s, 1H), 7.75 (s, 1H), 6.98-6.92 (m, 2H), 6.88 (d, J=8.4 Hz, 2H), 6.71 (d, J=8.4 Hz, 1H), 6.58-6.49 (m, 2H), 4.63-4.58 (m, 1H), 4.48 (s, 2H), 4.28-4.20 (m, 1H), 3.82-3.75 (m, 1H), 3.67-3.40 (m, 3H), 2.84-2.77 (m, 1H), 1.53-1.30 (m, 3H). Mass (m/z): 435.5 [M+H]⁺.

### Compound 554

### 7-((4-(4-fluoropiperidin-1-yl)phenyl)amino)-2H-pyrido[3,2-b][1,4]oxazin-3(4H)-one

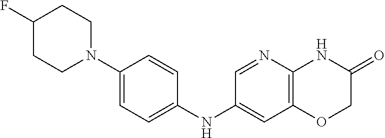 [SMILES: O=C1COc2cc(Nc3ccc(N4CCC(F)CC4)cc3)cnc2N1]

The titled compound 554 (23.9 mg, 13.5%) as a purple solid was prepared according to the procedure outlined for compound 1. ¹H NMR (400 MHz, DMSO-d₆) δ 10.98 (s, 1H), 7.86 (s, 1H), 7.60 (s, 1H), 6.96-6.88 (m, 5H), 4.97-4.68 (m, 1H), 4.57 (s, 2H), 3.26-3.22 (m, 2H), 3.04-3.00 (m, 2H), 2.06-1.90 (m, 2H), 1.86-1.78 (m, 2H). Mass (m/z): 343.5 [M+H]⁺.

### Compound 555

### 8-((4-(4-hydroxy-4-(trifluoromethyl)piperidin-1-yl)-2-methylphenyl) amino)-2,3-dihydrobenzo[b][1,4]oxazepin-4(5H)-one

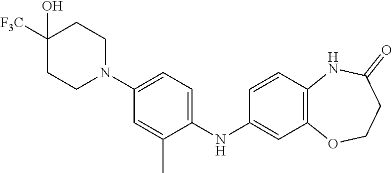 [SMILES: Cc1cc(N2CCC(O)(C(F)(F)F)CC2)ccc1Nc1ccc2c(c1)OCCC(=O)N2]

The titled compound 555 (16.6 mg, 42%) as an off-white solid was prepared according to the procedure outlined for compound 1. ¹H NMR (400 MHz, DMSO-d₆) δ 9.33 (s, 1H), 7.17 (s, 1H), 6.98 (d, J=8.4 Hz, 1H), 6.86 (s, 1H), 6.79 (d, J=7.6 Hz, 2H), 6.36 (d, J=8.8 Hz, 1H), 6.26 (s, 1H), 5.94 (s, 1H), 4.32 (t, J=6.4 Hz, 2H), 3.53 (d, J=12.0 Hz, 2H), 2.89 (t, J=12.0 Hz, 2H), 2.11 (s, 3H), 1.84-1.68 (m, 4H). Mass (m/z): 436.5 [M+H]⁺.

### Compound 556

### 8-((4-(4-hydroxy-4-(trifluoromethyl)piperidin-1-yl)phenyl)amino)-2,3-dihydrobenzo[b][1,4]oxazepin-4(5H)-one

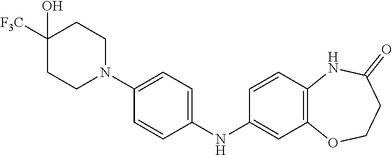 [SMILES: O=C1CCOc2cc(Nc3ccc(N4CCC(O)(C(F)(F)F)CC4)cc3)ccc2N1]

The titled compound 556 (11.7 mg, 30%) as a grey solid was prepared according to the procedure outlined for compound 1. ¹H NMR (400 MHz, DMSO-d₆) δ 9.41 (s, 1H), 7.77 (s, 1H), 6.96 (d, J=8.4 Hz, 2H), 6.91 (d, J=8.4 Hz, 2H), 6.84 (d, J=8.4 Hz, 1H), 6.63-6.52 (m, 2H), 5.93 (s, 1H), 4.34 (t, J=6.4 Hz, 2H), 3.45 (d, J=12.0 Hz, 2H), 2.87 (t, J=12.0 Hz, 2H), 2.62-2.58 (m, 2H), 1.89-1.70 (m, 4H). Mass (m/z): 422.5 [M+H]⁺.

### Compound 557

### 8-((6-(2-ethylmorpholino)-2-methylpyridin-3-yl)amino)-2,3-dihydrobenzo[b][1,4]oxazepin-4(5H)-one

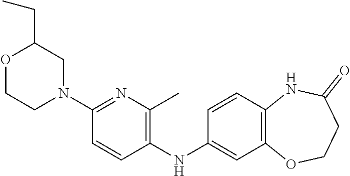 [SMILES: CCC1CN(c2ccc(Nc3ccc4c(c3)OCCC(=O)N4)c(C)n2)CCO1]

The titled compound 557 (10 mg, 24%) as a white solid was prepared according to the procedure outlined for compound 1. ¹H NMR (400 MHz, DMSO-d₆) δ 9.34 (s, 1H), 7.32 (d, J=8.8 Hz, 1H), 7.25 (s, 1H), 6.79 (d, J=8.8 Hz, 1H), 6.67 (d, J=8.8 Hz, 1H), 6.29 (d, J=8.8 Hz, 1H), 6.18 (s, 1H), 4.32 (t, J=5.2 Hz, 2H), 4.05 (d, J=12.4 Hz, 1H), 3.95 (t, J=10.8 Hz, 2H), 3.54 (t, J=11.6 Hz, 1H), 2.76-2.72 (m, 1H), 2.62-2.58 (m, 1H), 2.48-2.37 (m, 3H), 2.22 (s, 3H), 1.50 (p, J=8.0 Hz, 2H), 0.95 (t, J=7.6 Hz, 3H). Mass (m/z): 383.5 [M+H]⁺.

### Compound 558

### 7-(4-(4-fluoropiperidin-1-yl)-2-methylphenylamino)-2H-pyrido[3,2-b][1,4]oxazin-3(4H)-one

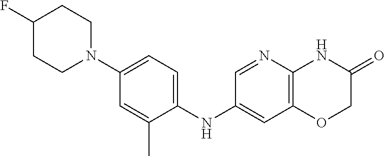 [SMILES: Cc1cc(N2CCC(F)CC2)ccc1Nc1cnc2c(c1)OCC(=O)N2]

The titled compound 558 (11 mg, 6.47%) as a yellow solid was prepared according to the procedure outlined for compound 1. ¹H NMR (400 MHz, DMSO-d₆) δ 10.89 (s, 1H), 7.37 (s, 1H), 7.26 (s, 1H), 6.97 (d, J=8.8 Hz, 1H), 6.87 (s, 1H), 6.77 (d, J=8.8 Hz, 1H), 6.60 (s, 1H), 4.95-4.78 (m, 1H), 4.54 (s, 2H), 3.29-3.27 (m, 2H), 3.11-2.98 (m, 2H), 2.13 (s, 3H), 2.05-1.88 (m, 2H), 1.85-1.70 (m, 2H). Mass (m/z): 357.1 [M+H]⁺.

### Compound 559

### 7-((4-(4-(2-methoxypropan-2-yl)piperidin-1-yl)-2-methylphenyl) amino)-2H-benzo[b][1,4]oxazin-3(4H)-one

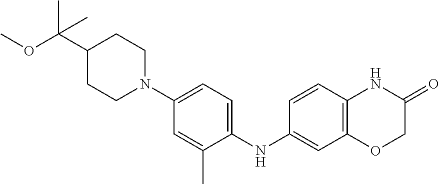 [SMILES: COC(C)(C)C1CCN(c2ccc(Nc3ccc4c(c3)OCC(=O)N4)c(C)c2)CC1]

The titled compound 559 (37.8 mg, 24.2%) as an off-white solid was prepared according to the procedure outlined for compound 1. ¹H NMR (400 MHz, DMSO-d₆) δ 10.37 (s, 1H), 7.05 (s, 1H), 6.94 (d, J=8.8 Hz, 1H), 6.80 (s, 1H), 6.72 (d, J=8.8 Hz, 1H), 6.65 (d, J=8.4 Hz, 1H), 6.29-6.20 (m, 2H), 4.44 (s, 2H), 3.65 (d, J=11.6 Hz, 2H), 3.10 (s, 3H), 2.10 (s, 3H), 1.69 (d, J=12.4 Hz, 2H), 1.55-1.44 (m, 1H), 1.41-1.27 (m, 2H), 1.06 (s, 6H). Mass (m/z): 410.2 [M+H]⁺.

### Compound 560

### 7-((4-(4-(2-methoxypropan-2-yl)piperidin-1-yl)phenyl)amino)-2H-benzo[b][1,4]oxazin-3(4H)-one

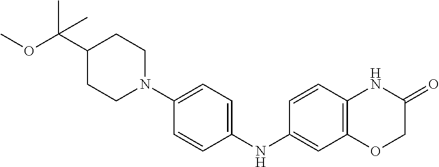 [SMILES: COC(C)(C)C1CCN(c2ccc(Nc3ccc4c(c3)OCC(=O)N4)cc2)CC1]

The titled compound 560 (13 mg, 8.2%) as a grey solid was prepared according to the procedure outlined for compound 1. ¹H NMR (400 MHz, DMSO-d₆) δ 10.44 (s, 1H), 7.66 (s, 1H), 6.95-6.88 (m, 2H), 6.85 (d, J=7.2 Hz, 2H), 6.73-6.67 (m, 1H), 6.55-6.47 (m, 2H), 4.47 (d, J=2.4 Hz, 2H), 3.58 (d, J=11.6 Hz, 2H), 3.09 (d, J=2.4 Hz, 3H), 2.48-2.42 (m, 2H), 1.69 (d, J=12.4 Hz, 2H), 1.54-1.44 (m, 1H), 1.35 (q, J=12.4 Hz, 2H), 1.10 (s, 6H). Mass (m/z): 396.2 [M+H]⁺.

### Compound 561

### 7-((4-(2-methyl-6-(trifluoromethyl)morpholino)phenyl)amino)-2H-benzo[b][1,4]oxazin-3(4H)-one

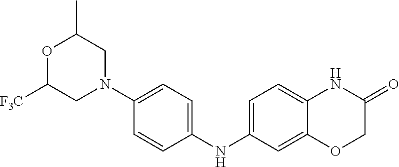 [SMILES: CC1CN(c2ccc(Nc3ccc4c(c3)OCC(=O)N4)cc2)CC(C(F)(F)F)O1]

The titled compound 561 (44 mg, 16.4%) as a white solid was prepared according to the procedure outlined for compound 1. ¹H NMR (400 MHz, DMSO-d₆) δ 10.46 (s, 1H), 7.76 (s, 1H), 6.96-6.86 (m, 4H), 6.72 (d, J=8.4 Hz, 1H), 6.57-6.46 (m, 2H), 4.48 (s, 2H), 4.29 (d, J=68.4 Hz, 1H), 3.87 (s, 1H), 3.58-3.46 (m, 1H), 3.23-3.06 (m, 2H), 2.82-2.75 (m, 1H), 1.24 (dd, J=23.2, 6.4 Hz, 3H). Mass (m/z): 408.2 [M+H]⁺.

### Compound 562

### 7-((2-methyl-4-(2-methyl-6-(trifluoromethyl)morpholino)phenyl)amino)-2H-benzo[b][1,4]oxazin-3(4H)-one

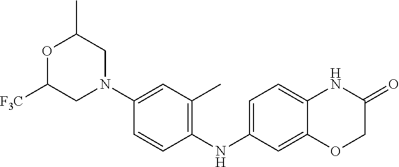 [SMILES: Cc1cc(N2CC(C)OC(C(F)(F)F)C2)ccc1Nc1ccc2c(c1)OCC(=O)N2]

The titled compound 562 (40 mg, 14.4%) as a light-yellow solid was prepared according to the procedure outlined for compound 1. ¹H NMR (400 MHz, DMSO-d₆) δ 10.38 (s, 1H), 7.11 (s, 1H), 6.99 (d, J=8.0 Hz, 1H), 6.87 (d, J=19.6 Hz, 1H), 6.82-6.72 (m, 1H), 6.65 (d, J=8.0 Hz, 1H), 6.32-6.22 (m, 2H), 4.48 (s, 2H), 4.29 (d, J=68.4 Hz, 1H), 3.87 (s, 1H), 3.58-3.46 (m, 1H), 3.23-3.06 (m, 2H), 2.82-2.75 (m, 1H), 2.12 (s, 3H), 1.24 (dd, J=23.2, 6.4 Hz, 3H). Mass (m/z): 422.2 [M+H]⁺.

### Compound 563

### 7-((2-methyl-4-(pyrrolidin-1-yl)phenyl)amino)-2H-benzo[b][1,4]oxazin-3(4H)-one

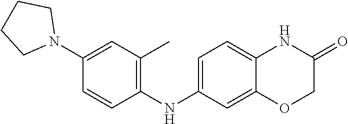 [SMILES: Cc1cc(N2CCCC2)ccc1Nc1ccc2c(c1)OCC(=O)N2]

The titled compound 563 (14.8 mg, 13.7%) as a grey solid was prepared according to the procedure outlined for compound 1. ¹H NMR (400 MHz, DMSO-d₆) δ 10.31 (s, 1H), 6.98 (s, 1H), 6.90 (d, J=8.4 Hz, 1H), 6.61 (d, J=8.4 Hz, 1H), 6.42 (d, J=2.8 Hz, 1H), 6.36 (dd, J=8.4, 2.8 Hz, 1H), 6.15 (dd, J=8.4, 2.4 Hz, 1H), 6.11 (d, J=2.4 Hz, 1H), 4.42 (s, 2H), 3.19 (q, J=6.0, 4.8 Hz, 4H), 2.08 (s, 3H), 1.98-1.88 (m, 4H). Mass (m/z): 324.2 [M+H]⁺.

### Compound 564

### 5-methoxy-7-((2-methyl-4-(4-(trifluoromethyl)piperidin-1-yl)phenyl) amino)-2H-benzo[b][1,4]oxazin-3(4H)-one

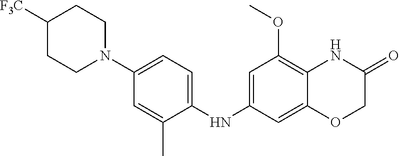 [SMILES: COc1cc(Nc2ccc(N3CCC(C(F)(F)F)CC3)cc2C)cc2c1NC(=O)CO2]

The titled compound 564 (10.2 mg, 5.99%) as a light purple solid was prepared according to the procedure outlined for compound 1. ¹H NMR (400 MHz, DMSO-d₆) δ 9.80 (s, 1H), 7.14 (s, 1H), 7.00 (d, J=8.8 Hz, 1H), 6.84 (d, J=2.8 Hz, 1H), 6.76 (dd, J=8.8, 2.8 Hz, 1H), 6.10 (d, J=2.0 Hz, 1H), 5.82 (d, J=2.0 Hz, 1H), 4.39 (s, 2H), 3.77-3.63 (m, 5H), 2.65 (dd, J=12.4, 10.0 Hz, 2H), 2.45 (dd, J=12.0, 3.6 Hz, 1H), 2.12 (s, 3H), 1.88 (d, J=12.4 Hz, 2H), 1.62-1.50 (m, 2H). Mass (m/z): 435.8 [M+H]⁺.

### Compound 565

### 5-methoxy-4-methyl-7-((2-methyl-4-(4-(trifluoromethyl)piperidin-1-yl) phenyl)amino)-2H-benzo[b][1,4]oxazin-3(4H)-one

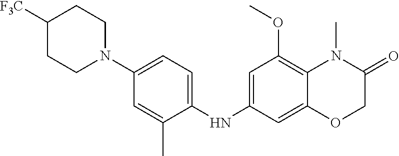 [SMILES: COc1cc(Nc2ccc(N3CCC(C(F)(F)F)CC3)cc2C)cc2c1N(C)C(=O)CO2]

The titled compound 565 (3.7 mg, 2.04%) as a dark purple solid was prepared according to the procedure outlined for compound 1. ¹H NMR (400 MHz, DMSO-d₆) δ 7.33 (s, 1H), 7.02 (d, J=8.8 Hz, 1H), 6.86 (d, J=2.8 Hz, 1H), 6.77 (dd, J=8.8, 2.8 Hz, 1H), 6.17 (d, J=2.4 Hz, 1H), 5.85 (d, J=2.4 Hz, 1H), 4.38 (s, 2H), 3.71 (d, J=14.4 Hz, 5H), 3.25 (s, 3H), 2.70-2.60 (m, 2H), 2.47-2.40 (m, 1H), 2.13 (s, 3H), 1.88 (d, J=12.0 Hz, 2H), 1.60-1.50 (m, 2H). Mass (m/z): 449.8 [M+H]⁺.

### Compound 566

### 3-methyl-6-({2-methyl-4-[4-(trifluoromethyl)piperidin-1-yl]phenyl}amino)-1,4-dihydroquinazolin-2-one

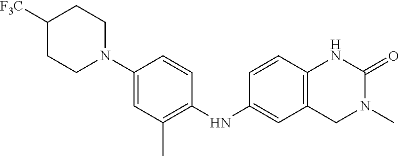 [SMILES: Cc1cc(N2CCC(C(F)(F)F)CC2)ccc1Nc1ccc2c(c1)CN(C)C(=O)N2]

The titled compound 566 (14.3 mg, 7%) as a light yellow solid was prepared according to the procedure outlined for compound 1. ¹H NMR (400 MHz, DMSO-d₆) δ 8.88 (s, 1H), 6.92 (d, J=8.8 Hz, 1H), 6.87 (s, 1H), 6.82 (d, J=2.4 Hz, 1H), 6.71 (dd, J=8.8, 2.8 Hz, 1H), 6.55 (dt, J=8.4, 5.2 Hz, 2H), 6.45 (s, 1H), 4.26 (s, 2H), 3.65 (d, J=12.4 Hz, 2H), 2.81 (s, 3H), 2.62 (t, J=11.2 Hz, 2H), 2.47-2.37 (m, 1H), 2.11 (s, 3H), 1.87 (d, J=11.6 Hz, 2H), 1.56 (tt, J=12.4, 6.4 Hz, 2H). Mass (m/z): 418.9 [M+H]⁺.

### Compound 567

### tert-butyl 6-((2-methyl-4-(4-(trifluoromethyl) piperidin-1-yl) phenyl)amino)-3H-imidazo[4,5-b] pyridine-3-carboxylate

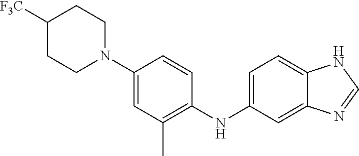 [SMILES: Cc1cc(N2CCC(C(F)(F)F)CC2)ccc1Nc1ccc2[nH]cnc2c1]

The titled compound 567 (32.1 mg, 40%) as a grey solid was prepared according to the procedure outlined for compound 1. ¹H NMR (400 MHz, DMSO-d₆) δ 8.38 (s, 1H), 7.44 (d, J=8.8 Hz, 1H), 7.28 (s, 1H), 7.03 (d, J=8.4 Hz, 1H), 6.89 (s, 1H), 6.81 (dd, J=14.0, 9.2 Hz, 1H), 6.69 (s, 1H), 3.71 (d, J=11.6 Hz, 2H), 2.67 (t, J=11.6 Hz, 2H), 2.50-2.33 (m, 1H), 2.15 (s, 3H), 1.89 (d, J=12.0 Hz, 2H), 1.58 (dd, J=12.4, 2.8 Hz, 2H). Mass (m/z): 375 [M+H]⁺.

### Compound 568

### 8-methyl-7-((4-(4-(trifluoromethyl) piperidin-1-yl)phenyl) amino)-2H-benzo[b] [1,4] oxazin-3(4H)-one

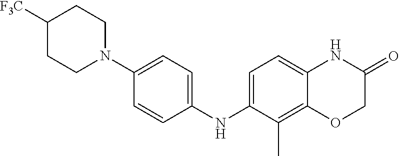 [SMILES: Cc1c(Nc2ccc(N3CCC(C(F)(F)F)CC3)cc2)ccc2c1OCC(=O)N2]

The titled compound 568 (38.9 mg, 22.66%) as a dark purple solid was prepared according to the procedure outlined for compound 1. ¹H NMR (400 MHz, DMSO-d₆) δ 10.48 (s, 1H), 6.99 (s, 1H), 6.74 (m, 6H), 4.54 (s, 2H), 3.55 (d, J=11.2 Hz, 2H), 2.60 (m, 2H), 2.40 (s, 1H), 2.05 (d, J=20.4 Hz, 3H), 1.87 (d, J=10.4 Hz, 2H), 1.57 (d, J=10.4 Hz, 2H). Mass (m/z): 406 [M+H]⁺.

### Compound 569

### 7-((2-methyl-4-(4-(2,2,2-trifluoroethyl) piperazin-1-yl)phenyl) amino)-2H-benzo[b][1,4] oxazin-3(4H)-one

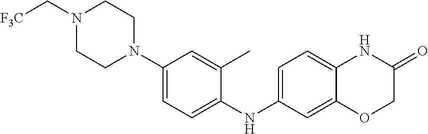 [SMILES: Cc1cc(N2CCN(CC(F)(F)F)CC2)ccc1Nc1ccc2c(c1)OCC(=O)N2]

The titled compound 569 (69.2 mg, 29%) as a purple solid was prepared according to the procedure outlined for compound 1. ¹H NMR (400 MHz, DMSO-d₆) δ 10.38 (s, 1H), 7.08 (s, 1H), 6.96 (d, J=8.8 Hz, 1H), 6.81 (d, J=2.4 Hz, 1H), 6.72 (dd, J=8.8, 2.4 Hz, 1H), 6.65 (d, J=8.4 Hz, 1H), 6.31-6.21 (m, 2H), 4.45 (s, 2H), 3.23 (q, J=10.0 Hz, 2H), 3.12-3.03 (m, 4H), 2.78-2.72 (m, 4H), 2.11 (s, 3H). Mass (m/z): 421 [M+H]⁺.

### Compound 570

### 2-(hydroxymethyl)-4-methyl-7-((2-methyl-4-(4-(trifluoromethyl) piperidin-1-yl)phenyl)amino)-2H-benzo[b][1,4]oxazin-3(4H)-one

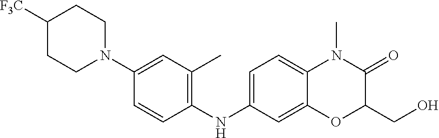 [SMILES: Cc1cc(N2CCC(C(F)(F)F)CC2)ccc1Nc1ccc2c(c1)OC(CO)C(=O)N2C]

The titled compound 570 (2.1 mg, 3.44%) as a white solid was prepared according to the procedure outlined for compound 1. ¹H NMR (400 MHz, DMSO-d₆) δ 7.16-7.13 (m, 1H), 6.97-6.94 (m, 1H), 6.84-6.81 (m, 2H), 6.75-6.71 (m, 1H), 6.31-6.27 (m, 1H), 6.23-6.22 (m, 1H), 5.01-4.93 (m, 1H), 4.53-4.50 (m, 1H), 3.72-3.63 (m, 4H), 3.15 (s, 3H), 2.65-2.58 (m, 2H), 2.44-2.37 (m, 1H), 2.08 (s, 3H), 1.84 (d, J=11.6 Hz, 2H), 1.57-1.48 (m, 2H). Mass (m/z): 449.8 [M+H]⁺.

### Compound 571

### 5-((2-methyl-4-(4-(trifluoromethyl)piperidin-1-yl)phenyl)amino)-1H-indole-3-carboxamide

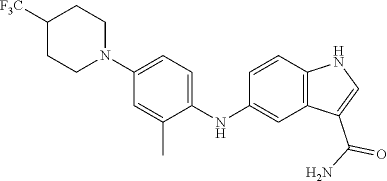 [SMILES: Cc1cc(N2CCC(C(F)(F)F)CC2)ccc1Nc1ccc2[nH]cc(C(N)=O)c2c1]

The titled compound 571 (18 mg, 10.8%) as a black solid was prepared according to the procedure outlined for compound 1. ¹H NMR (400 MHz, DMSO-d₆) δ 11.20 (d, J=2.4 Hz, 1H), 7.85 (d, J=3.2 Hz, 1H), 7.53 (d, J=2.0 Hz, 1H), 7.17 (d, J=8.8 Hz, 1H), 6.91 (d, J=8.8 Hz, 1H), 6.80-6.76 (m, 2H), 6.71-6.64 (m, 2H), 3.60 (d, J=12.0 Hz, 2H), 2.65-2.54 (m, 2H), 2.38 (dd, J=8.0, 3.6 Hz, 1H), 2.12 (s, 3H), 1.84 (d, J=12.0 Hz, 2H), 1.54 (dd, J=12.4, 3.6 Hz, 2H). Mass (m/z): 416.7 [M+H]⁺.

### Compound 572

### 7-(4-(3-fluoroazetidin-1-yl)-2-methylphenylamino)-2H-benzo[b][1,4]oxazin-3(4H)-one

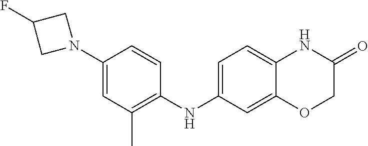 [SMILES: Cc1cc(N2CC(F)C2)ccc1Nc1ccc2c(c1)OCC(=O)N2]

The titled compound 572 (23.5 mg, 17.0%) as a white solid was prepared according to the procedure outlined for compound 1. ¹H NMR (400 MHz, DMSO-d₆) δ 10.36 (s, 1H), 7.05 (s, 1H), 6.94 (d, J=8.4 Hz, 1H), 6.64 (d, J=8.4 Hz, 1H), 6.38 (s, 1H), 6.31 (d, J=8.8 Hz, 1H), 6.25-6.11 (m, 2H), 5.58-5.34 (m, 1H), 4.44 (s, 2H), 4.19-4.05 (m, 2H), 3.88-3.75 (m, 2H), 2.09 (s, 3H). Mass (m/z): 328.2 [M+H]⁺.

### Compound 573

### 6-{[6-(2-ethylmorpholin-4-yl)-2-methylpyridin-3-yl]amino}-3,4-dihydro-1H-quinazolin-2-one

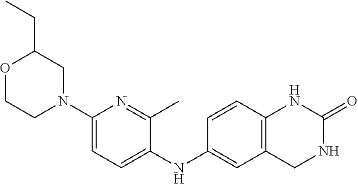 [SMILES: CCC1CN(c2ccc(Nc3ccc4c(c3)CNC(=O)N4)c(C)n2)CCO1]

The titled compound 573 (26.6 mg, 5.05%) as a light-yellow solid was prepared according to the procedure outlined for compound 461. ¹H NMR (400 MHz, DMSO-d₆) δ 8.68 (s, 1H), 7.27 (d, J=8.8 Hz, 1H), 6.93 (s, 1H), 6.63 (d, J=8.8 Hz, 1H), 6.58 (d, J=8.4 Hz, 2H), 6.44 (dd, J=8.4, 2.4 Hz, 1H), 6.35 (d, J=1.6 Hz, 1H), 4.18 (s, 2H), 4.02 (d, J=12.0 Hz, 1H), 3.93 (d, J=10.8 Hz, 2H), 3.54 (dd, J=11.6, 9.2 Hz, 1H), 3.37 (s, 1H), 2.76-2.67 (m, 1H), 2.40 (dd, J=12.4, 10.4 Hz, 1H), 2.22 (s, 3H), 1.49 (dd, J=14.4, 7.2 Hz, 2H), 0.95 (t, J=7.6 Hz, 3H). Mass (m/z): 367.9 [M+H]⁺.

### Compound 574

### 8-methyl-7-((2-methyl-4-(4-(trifluoromethyl) piperidin-1-yl) phenyl)amino)-2H-benzo[b][1,4]oxazin-3(4H)-one

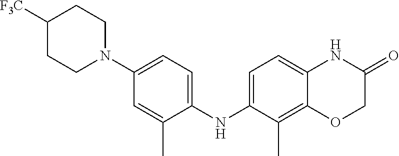 [SMILES: Cc1cc(N2CCC(C(F)(F)F)CC2)ccc1Nc1ccc2c(c1C)OCC(=O)N2]

The titled compound 574 (20.0 mg, 10.4%) as a yellow solid was prepared according to the procedure outlined for compound 1. ¹H NMR (400 MHz, DMSO-d₆) δ 7.16-7.13 (m, 1H), 6.97-6.94 (m, 1H), 6.84-6.81 (m, 2H), 6.75-6.71 (m, 1H), 6.31-6.27 (m, 1H), 6.23-6.22 (m, 1H), 5.01-4.93 (m, 1H), 4.53-4.50 (m, 1H), 3.72-3.63 (m, 4H), 3.15 (s, 3H), 2.63 (m, 2H), 2.44-2.37 (m, 1H), 2.08 (s, 3H), 1.84 (d, J=11.6 Hz, 2H), 1.57-1.48 (m, 2H). Mass (m/z): 420 [M+H]⁺.

### Compound 575

### 7-((2-methyl-4-(4-methylpiperidin-1-yl) phenyl) amino)-2H-pyrido[3,2-b][1,4]oxazin-3(4H)-one

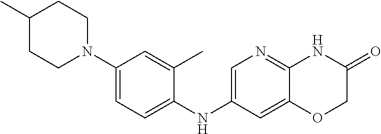 [SMILES: Cc1cc(N2CCC(C)CC2)ccc1Nc1cnc2c(c1)OCC(=O)N2]

The titled compound 575 (31.2 mg, 33.80%) as a grey solid was prepared according to the procedure outlined for compound 1. ¹H NMR (400 MHz, DMSO-d₆) δ 10.85 (s, 1H), 7.32 (d, J=2.4 Hz, 1H), 7.20 (s, 1H), 6.91 (d, J=8.8 Hz, 1H), 6.79 (d, J=2.8 Hz, 1H), 6.69 (dd, J=8.8, 2.8 Hz, 1H), 6.54 (d, J=2.4 Hz, 1H), 4.50 (s, 2H), 3.54 (d, J=12.4 Hz, 2H), 2.54 (d, J=2.4 Hz, 2H), 2.08 (s, 3H), 1.64 (d, J=12.4 Hz, 2H), 1.45-1.39 (m, 1H), 1.19 (dd, J=12.0, 3.6 Hz, 2H), 0.90 (d, J=6.4 Hz, 3H). Mass (m/z): 353 [M+H]⁺.

### Compound 576

### 7-((4-(3,3-difluoroazetidin-1-yl)-2-methylphenyl)amino)-2H-benzo[b][1,4]oxazin-3(4H)-one

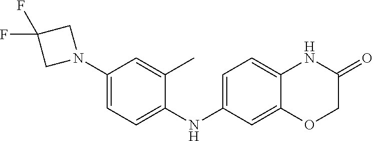 [SMILES: Cc1cc(N2CC(F)(F)C2)ccc1Nc1ccc2c(c1)OCC(=O)N2]

The titled compound 576 (4.6 mg, 1.45%) as an off-white solid was prepared according to the procedure outlined for compound 1. ¹H NMR (400 MHz, DMSO-d₆) δ 10.37 (s, 1H), 7.10 (s, 1H), 6.98 (d, J=8.4 Hz, 1H), 6.65 (d, J=8.4 Hz, 1H), 6.46 (d, J=2.4 Hz, 1H), 6.38 (dd, J=8.4, 2.8 Hz, 1H), 6.26-6.18 (m, 2H), 4.44 (s, 2H), 4.21 (t, J=12.4 Hz, 4H), 2.10 (s, 3H). Mass (m/z): 345.8 [M+H]⁺.

### Compound 577

### 7-((2-methyl-4-(3-methylazetidin-1-yl)phenyl)amino)-2H-benzo[b][1,4]oxazin-3(4H)-one

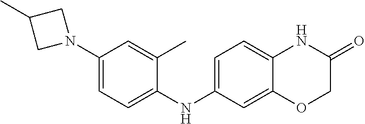 [SMILES: Cc1cc(N2CC(C)C2)ccc1Nc1ccc2c(c1)OCC(=O)N2]

The titled compound 577 (3.3 mg, 1.15%) as a white solid was prepared according to the procedure outlined for compound 1. ¹H NMR (400 MHz, DMSO-d₆) δ 10.34 (s, 1H), 7.01 (s, 1H), 6.90 (d, J=8.4 Hz, 1H), 6.62 (d, J=8.4 Hz, 1H), 6.28 (d, J=2.4 Hz, 1H), 6.23-6.16 (m, 2H), 6.13 (d, J=2.4 Hz, 1H), 4.43 (s, 2H), 3.90 (t, J=7.2 Hz, 2H), 3.31 (d, J=6.8 Hz, 1H), 2.74 (dd, J=13.6, 6.4 Hz, 1H), 2.06 (s, 3H), 1.22 (d, J=6.8 Hz, 3H). Mass (m/z): 323.9 [M+H]⁺.

### Compound 578

### 7-((4-(3-methoxyazetidin-1-yl)-2-methylphenyl)amino)-2H-benzo[b][1,4]oxazin-3(4H)-one

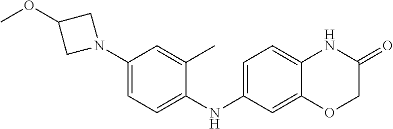 [SMILES: COC1CN(c2ccc(Nc3ccc4c(c3)OCC(=O)N4)c(C)c2)C1]

The titled compound 578 (2.1 mg, 1.07%) as a white solid was prepared according to the procedure outlined for compound 1. ¹H NMR (400 MHz, DMSO-d₆) δ 10.34 (s, 1H), 7.03 (s, 1H), 6.91 (d, J=8.4 Hz, 1H), 6.63 (d, J=8.4 Hz, 1H), 6.33 (d, J=2.4 Hz, 1H), 6.26 (dd, J=8.4, 2.8 Hz, 1H), 6.21-6.12 (m, 2H), 4.43 (s, 2H), 4.31-4.26 (m, 1H), 4.05-3.96 (m, 2H), 3.53 (dd, J=8.4, 4.4 Hz, 2H), 3.23 (s, 3H), 2.07 (s, 3H). Mass (m/z): 339.8 [M+H]⁺.

### Compound 579

### 2-(5-((2-methyl-4-(4-(trifluoromethyl)piperidin-1-yl)phenyl)amino) isoindolin-2-yl)acetamide

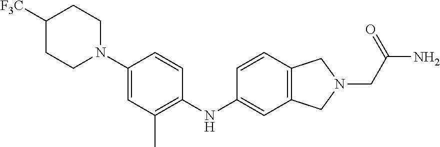 [SMILES: Cc1cc(N2CCC(C(F)(F)F)CC2)ccc1Nc1ccc2c(c1)CN(CC(N)=O)C2]

The titled compound 579 (2.3 mg, 3.5%) as a white solid was prepared according to the procedure outlined for compound 1. ¹H NMR (400 MHz, DMSO-d₆) δ 7.28 (s, 1H), 7.09 (d, J=7.6 Hz, 2H), 6.96 (t, J=9.2 Hz, 2H), 6.84 (s, 1H), 6.75 (d, J=8.8 Hz, 1H), 6.51 (d, J=5.6 Hz, 2H), 3.83 (s, 4H), 3.69 (d, J=12.4 Hz, 2H), 3.22 (s, 2H), 2.71-2.63 (m, 3H), 2.11 (s, 3H), 1.88 (d, J=12.4 Hz, 2H), 1.56 (d, J=12.4 Hz, 2H). Mass (m/z): 433.2 [M+H]⁺.

### Compound 580

### (S)-7-((2-methyl-4-(3-methyl-4-(2,2,2-trifluoroethyl)piperazin-1-yl) phenyl)amino)-2H-benzo[b][1,4]oxazin-3(4H)-one

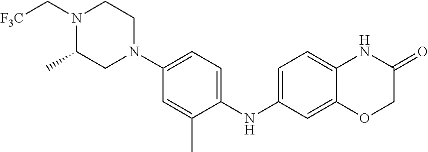 [SMILES: Cc1cc(N2CCN(CC(F)(F)F)[C@@H](C)C2)ccc1Nc1ccc2c(c1)OCC(=O)N2]

The titled compound 580 (23.7 mg, 15%) as a grey solid was prepared according to the procedure outlined for compound 1. ¹H NMR (400 MHz, DMSO-d₆) δ 10.37 (s, 1H), 7.07 (s, 1H), 6.95 (d, J=8.4 Hz, 1H), 6.81 (s, 1H), 6.72 (d, J=8.8 Hz, 1H), 6.65 (d, J=8.4 Hz, 1H), 6.30-6.21 (m, 2H), 4.44 (s, 2H), 3.49-3.34 (m, 2H), 3.20-3.06 (m, 1H), 3.03-2.95 (m, 1H), 2.80-2.68 (m, 3H), 2.48-2.42 (m, 2H), 2.11 (s, 3H), 1.12-1.06 (m, 3H). Mass (m/z): 435.5 [M+H]⁺.

### Compound 581

### (S)-7-((4-(3-methyl-4-(2,2,2-trifluoroethyl)piperazin-1-yl)phenyl) amino)-2H-benzo[b][1,4]oxazin-3(4H)-one

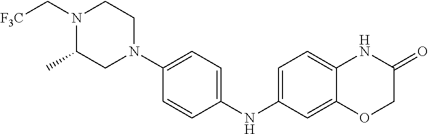 [SMILES: C[C@H]1CN(c2ccc(Nc3ccc4c(c3)OCC(=O)N4)cc2)CCN1CC(F)(F)F]

The titled compound 581 (42.7 mg, 22.7%) as a light brown solid was prepared according to the procedure outlined for compound 1. ¹H NMR (400 MHz, DMSO-d₆) δ 10.44 (s, 1H), 7.68 (s, 1H), 6.93 (d, J=8.4 Hz, 2H), 6.85 (d, J=8.4 Hz, 2H), 6.70 (d, J=8.4 Hz, 1H), 6.56-6.47 (m, 2H), 4.47 (s, 2H), 3.50-3.37 (m, 1H), 3.31-3.21 (m, 2H), 3.17-3.06 (m, 1H), 3.02-2.95 (m, 1H), 2.77-2.71 (m, 3H), 2.48-2.41 (m, 2H), 1.11-1.05 (m, 3H). Mass (m/z): 421.5 [M+H]⁺.

### Compound 582

### 7-((4-(4-(trifluoromethyl)piperidin-1-yl)phenyl)amino)-2H-benzo[b][1,4]thiazin-3(4H)-one

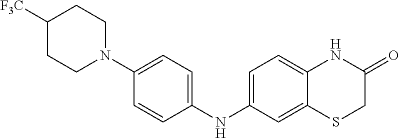 [SMILES: O=C1CSc2cc(Nc3ccc(N4CCC(C(F)(F)F)CC4)cc3)ccc2N1]

The titled compound 582 (21.6 mg, 12.9%) as an off-white solid was prepared according to the procedure outlined for compound 1. ¹H NMR (400 MHz, DMSO-d₆) δ 10.29 (s, 1H), 7.74 (s, 1H), 6.97-6.86 (m, 4H), 6.84-6.78 (m, 2H), 6.78-6.72 (m, 1H), 3.62 (d, J=12.0 Hz, 2H), 3.39 (s, 2H), 2.64-2.57 (m, 2H), 2.44-2.37 (m, 1H), 1.88 (d, J=12.8 Hz, 2H), 1.65-1.49 (m, 2H). Mass (m/z): 408.5 [M+H]⁺.

### Compound 583

### 7-((2-methyl-4-(3-(trifluoromethyl)azetidin-1-yl)phenyl)amino)-2H-benzo[b][1,4]oxazin-3(4H)-one

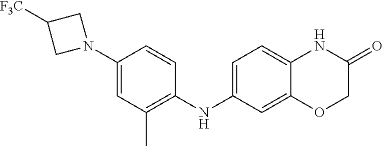 [SMILES: Cc1cc(N2CC(C(F)(F)F)C2)ccc1Nc1ccc2c(c1)OCC(=O)N2]

The titled compound 583 (19.1 mg, 11.6%) as a white solid was prepared according to the procedure outlined for compound 1. ¹H NMR (400 MHz, DMSO-d₆) δ 10.35 (s, 1H), 7.06 (s, 1H), 6.95 (d, J=8.4 Hz, 1H), 6.63 (d, J=8.4 Hz, 1H), 6.38 (s, 1H), 6.31 (d, J=8.8 Hz, 1H), 6.24-6.14 (m, 2H), 4.44 (s, 2H), 4.00 (t, J=8.4 Hz, 2H), 3.80 (t, J=6.8 Hz, 2H), 3.70-3.66 (m, 1H), 2.09 (s, 3H). Mass (m/z): 378.5 [M+H]⁺.

### Compound 584

### 6-((2-methyl-4-(3-(trifluoromethyl)pyrrolidin-1-yl)phenyl)amino)-2H-benzo[b][1,4]oxazin-3(4H)-one

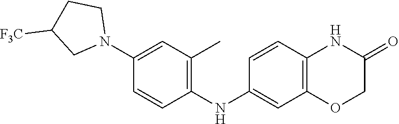 [SMILES: Cc1cc(N2CCC(C(F)(F)F)C2)ccc1Nc1ccc2c(c1)OCC(=O)N2]

The titled compound 584 (6.8 mg, 6.1%) as brown oil was prepared according to the procedure outlined for compound 1. ¹H NMR (400 MHz, DMSO-d₆) δ 10.40 (s, 1H), 7.07-6.89 (m, 2H), 6.69 (d, J=8.8 Hz, 1H), 6.52 (s, 1H), 6.44 (d, J=8.4 Hz, 1H), 6.25-6.08 (m, 2H), 4.39 (s, 2H), 3.47 (d, J=9.2 Hz, 1H), 3.26 (d, J=8.5 Hz, 2H), 2.15-2.0 (m, 4H), 1.23 (s, 2H). Mass (m/z): 392.4 [M+H]⁺.

### Compound 585

### 7-((4-(azetidin-1-yl)-2-methylphenyl)amino)-2H-benzo[b][1,4]oxazin-3(4H)-one

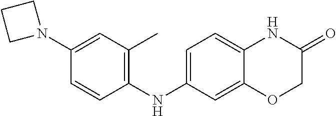 [SMILES: Cc1cc(N2CCC2)ccc1Nc1ccc2c(c1)OCC(=O)N2]

The titled compound 585 (22.6 mg, 14.8%) as pink oil was prepared according to the procedure outlined for compound 1. ¹H NMR (400 MHz, DMSO-d₆) δ 10.34 (s, 1H), 7.01 (s, 1H), 6.90 (d, J=8.0 Hz, 1H), 6.63 (d, J=8.0 Hz, 1H), 6.29 (s, 1H), 6.26-6.06 (m, 3H), 4.43 (d, J=2.8 Hz, 2H), 3.75 (t, J=7.6 Hz, 4H), 2.27 (t, J=7.6 Hz, 2H), 2.07 (s, 3H). Mass (m/z): 310.2 [M+H]⁺.

### Compound 586

### 8-((4-(azetidin-1-yl)-2-methylphenyl)amino)-2,3-dihydrobenzo[b][1,4]oxazepin-4(5H)-one

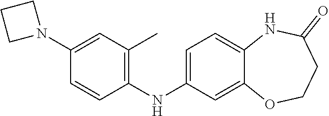 [SMILES: Cc1cc(N2CCC2)ccc1Nc1ccc2c(c1)OCCC(=O)N2]

The titled compound 586 (19.3 mg, 58.5%) as pink oil was prepared according to the procedure outlined for compound 1. ¹H NMR (400 MHz, DMSO-d₆) δ 9.30 (s, 1H), 7.11 (d, J=2.8 Hz, 1H), 6.92 (dd, J=8.4, 2.4 Hz, 1H), 6.76 (dd, J=8.8, 2.4 Hz, 1H), 6.35-6.19 (m, 3H), 6.16 (s, 1H), 4.31 (q, J=5.2 Hz, 2H), 3.83-3.67 (m, 4H), 2.56 (s, 2H), 2.27 (t, J=7.6 Hz, 2H), 2.07 (d, J=2.8 Hz, 3H). Mass (m/z): 324.2 [M+H]⁺.

### Compound 587

### 7-((2-methyl-4-(3-(trifluoromethyl)pyrrolidin-1-yl)phenyl)amino)-2H-benzo[b][1,4]oxazin-3(4H)-one

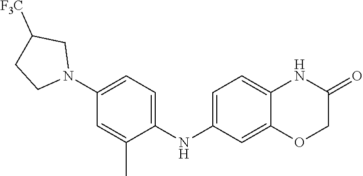 [SMILES: Cc1cc(N2CCC(C(F)(F)F)C2)ccc1Nc1ccc2c(c1)OCC(=O)N2]

The titled compound 587 (33.5 mg, 6.9%) as an off-white solid was prepared according to the procedure outlined for compound 1. ¹H NMR (400 MHz, DMSO-d₆) δ 10.34 (s, 1H), 7.03 (s, 1H), 6.97-6.90 (m, 1H), 6.66-6.59 (m, 1H), 6.52 (s, 1H), 6.44 (d, J=8.8 Hz, 1H), 6.21-6.12 (m, 2H), 4.43 (s, 2H), 3.53-3.44 (m, 1H), 3.40-3.35 (m, 2H), 3.32-3.23 (m, 2H), 2.30-2.25 (m, 1H), 2.14-2.01 (m, 4H). Mass (m/z): 392.4 [M+H]⁺.

### Compound 588

### 7-((2-methyl-4-(3-(trifluoromethyl)pyrrolidin-1-yl)phenyl)amino)-2H-benzo[b][1,4]oxazin-3(4H)-one

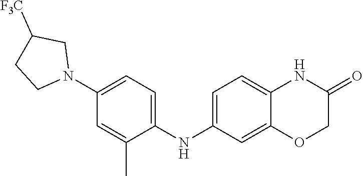 [SMILES: Cc1cc(N2CCC(C(F)(F)F)C2)ccc1Nc1ccc2c(c1)OCC(=O)N2]

The titled compound 588 (24.4 mg, 5%) as a purple solid was prepared according to the procedure outlined for compound 1. ¹H NMR (400 MHz, DMSO-d₆) δ 10.34 (s, 1H), 7.03 (s, 1H), 6.97-6.90 (m, 1H), 6.66-6.59 (m, 1H), 6.52 (s, 1H), 6.44 (d, J=8.8 Hz, 1H), 6.21-6.12 (m, 2H), 4.43 (s, 2H), 3.53-3.44 (m, 1H), 3.40-3.35 (m, 2H), 3.32-3.23 (m, 1H), 2.28 (d, J=12.0 Hz, 1H), 2.16-2.01 (m, 4H). Mass (m/z): 392.4 [M+H]⁺.

### Compound 589

### 7-((2-methyl-4-(3-(trifluoromethoxy)pyrrolidin-1-yl)phenyl)amino)-2H-benzo[b][1,4]oxazin-3(4H)-one

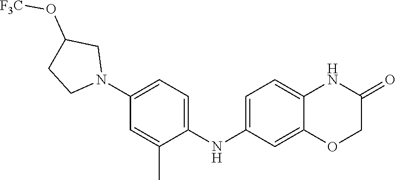 [SMILES: Cc1cc(N2CCC(OC(F)(F)F)C2)ccc1Nc1ccc2c(c1)OCC(=O)N2]

The titled compound 589 (8.7 mg, 5.5%) as an off-white solid was prepared according to the procedure outlined for compound 1. ¹H NMR (400 MHz, DMSO-d₆) δ 10.33 (s, 1H), 7.03 (s, 1H), 6.97-6.90 (m, 1H), 6.66-6.58 (m, 1H), 6.48 (s, 1H), 6.41 (d, J=8.8 Hz, 1H), 6.20-6.11 (m, 2H), 5.22 (s, 1H), 4.43 (s, 2H), 3.62-3.54 (m, 1H), 3.44-3.34 (m, 2H), 2.43-2.27 (m, 2H), 2.26-2.16 (m, 1H), 2.10 (s, 3H). Mass (m/z): 408.5 [M+H]⁺.

### Compound 590

### 5-(4-methoxybenzyl)-8-((2-methyl-4-(3-(trifluoromethyl)azetidin-1-yl) phenyl)amino)-2,3-dihydrobenzo[b][1,4]oxazepin-4(5H)-one

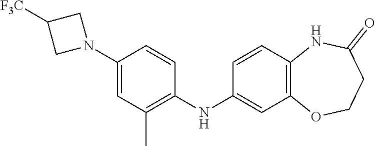 [SMILES: Cc1cc(N2CC(C(F)(F)F)C2)ccc1Nc1ccc2c(c1)OCCC(=O)N2]

The titled compound 590 (11 mg, 28%) as a purple solid was prepared according to the procedure outlined for compound 1. ¹H NMR (400 MHz, DMSO-d₆) δ 9.31 (s, 1H), 7.15 (s, 1H), 7.00-6.93 (m, 1H), 6.80-6.73 (m, 1H), 6.39 (s, 1H), 6.35-6.26 (m, 2H), 6.18 (s, 1H), 4.35-4.27 (m, 2H), 4.05-3.96 (m, 2H), 3.86-3.75 (m, 2H), 3.71-3.66 (m, 1H), 2.62-2.54 (m, 2H), 2.09 (s, 3H). Mass (m/z): 392.5 [M+H]⁺.

### Compound 591

### 8-((2-methyl-4-(3-(trifluoromethoxy)pyrrolidin-1-yl)phenyl)amino)-2,3-dihydrobenzo[b][1,4]oxazepin-4(5H)-one

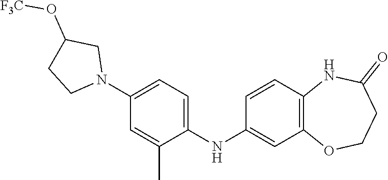 [SMILES: Cc1cc(N2CCC(OC(F)(F)F)C2)ccc1Nc1ccc2c(c1)OCCC(=O)N2]

The titled compound 591 (3.1 mg, 7.9%) as a light brown solid was prepared according to the procedure outlined for compound 1. ¹H NMR (400 MHz, CD₃OD) δ 6.99 (d, J=8.4 Hz, 1H), 6.76 (d, J=8.4 Hz, 1H), 6.51 (s, 1H), 6.45 (d, J=8.4 Hz, 1H), 6.33 (d, J=8.4 Hz, 1H), 6.24 (s, 1H), 5.12 (s, 1H), 4.41 (s, 2H), 3.64-3.60 (m, 1H), 3.47-3.40 (m, 3H), 2.69-2.61 (m, 2H), 2.33-2.29 (m, 2H), 2.16 (s, 3H). Mass (m/z): 422.5 [M+H]⁺.

### Compound 592

### 2-(aminomethyl)-7-((2-methyl-4-(4-(trifluoromethyl)piperidin-1-yl) phenyl)amino)-2H-benzo[b][1,4]oxazin-3(4H)-one

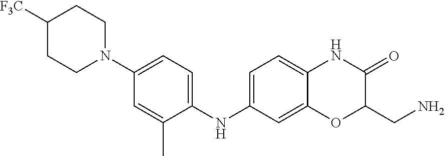 [SMILES: Cc1cc(N2CCC(C(F)(F)F)CC2)ccc1Nc1ccc2c(c1)OC(CN)C(=O)N2]

The titled compound 592 (10.9 mg, 26.8%) as a light brown solid was prepared according to the procedure outlined for compound 1. ¹H NMR (400 MHz, DMSO-d₆) δ 10.35 (s, 1H), 7.07 (s, 1H), 6.97 (d, J=8.8 Hz, 1H), 6.84 (s, 1H), 6.75 (d, J=8.8 Hz, 1H), 6.63 (d, J=8.4 Hz, 1H), 6.27 (d, J=11.2 Hz, 2H), 4.38-4.33 (m, 1H), 3.68 (d, J=12.0 Hz, 2H), 2.90 (s, 2H), 2.66-2.59 (m, 2H), 2.44-2.36 (m, 1H), 2.11 (s, 3H), 1.88 (d, J=12.8 Hz, 2H), 1.63-1.49 (m, 2H). Mass (m/z): 435.5 [M+H]⁺.

### Compound 593

### 7-((2-methyl-4-(4-(trifluoromethyl)piperidin-1-yl)phenyl)amino)-2H-benzo[b][1,4]thiazin-3(4H)-one

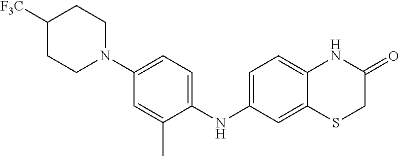 [SMILES: Cc1cc(N2CCC(C(F)(F)F)CC2)ccc1Nc1ccc2c(c1)SCC(=O)N2]

The titled compound 593 (28.7 mg, 15%) as a white solid was prepared according to the procedure outlined for compound 1. ¹H NMR (400 MHz, DMSO-d₆) δ 10.22 (s, 1H), 7.14 (s, 1H), 6.99-6.92 (m, 1H), 6.85 (s, 1H), 6.79-6.71 (m, 2H), 6.55-6.47 (m, 2H), 3.69 (d, J=12.0 Hz, 2H), 3.37 (s, 2H), 2.63 (d, J=12.4 Hz, 2H), 2.48-2.36 (m, 1H), 2.11 (d, J=2.8 Hz, 3H), 1.88 (d, J=12.4 Hz, 2H), 1.63-1.49 (m, 2H). Mass (m/z): 422.5 [M+H]⁺.

### Compound 594

### 5-methyl-8-((2-methyl-4-(4-(trifluoromethyl)piperidin-1-yl)phenyl) amino)-2,3-dihydrobenzo[b][1,4]oxazepin-4(5H)-one

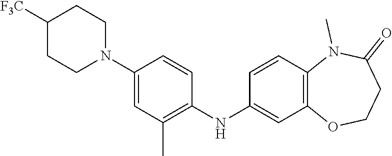 [SMILES: Cc1cc(N2CCC(C(F)(F)F)CC2)ccc1Nc1ccc2c(c1)OCCC(=O)N2C]

The titled compound 594 (25.8 mg, 18%) as a white solid was prepared according to the procedure outlined for compound 1. ¹H NMR (400 MHz, DMSO-d₆) δ 7.39 (s, 1H), 7.09 (d, J=8.4 Hz, 1H), 7.02 (d, J=8.4 Hz, 1H), 6.87 (s, 1H), 6.78 (d, J=8.4 Hz, 1H), 6.49 (d, J=8.4 Hz, 1H), 6.32 (s, 1H), 4.41-4.34 (m, 2H), 3.72 (d, J=12.0 Hz, 2H), 3.33 (s, 3H), 2.72-2.61 (m, 2H), 2.46-2.38 (m, 1H), 2.13 (s, 3H), 1.88 (d, J=12.8 Hz, 2H), 1.63-1.49 (m, 2H). Mass (m/z): 434.5 [M+H]⁺.

### Compound 595

### 7-((4-(3-fluoro-3-methylazetidin-1-yl)-2-methylphenyl)amino)-2H-benzo[b][1,4]oxazin-3(4H)-one

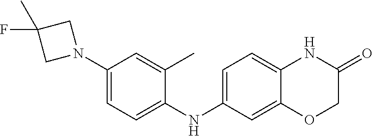 [SMILES: Cc1cc(N2CC(C)(F)C2)ccc1Nc1ccc2c(c1)OCC(=O)N2]

The titled compound 595 (37.7 mg, 13%) as a purple solid was prepared according to the procedure outlined for compound 1. ¹H NMR (400 MHz, DMSO-d₆) δ 10.35 (s, 1H), 7.05 (s, 1H), 6.94 (d, J=8.4 Hz, 1H), 6.63 (d, J=8.4 Hz, 1H), 6.37 (d, J=2.4 Hz, 1H), 6.30 (dd, J=8.4, 2.8 Hz, 1H), 6.21 (dd, J=8.4, 2.4 Hz, 1H), 6.16 (d, J=2.4 Hz, 1H), 4.44 (s, 2H), 3.86 (d, J=19.2 Hz, 4H), 2.08 (s, 3H), 1.62 (d, J=22.4 Hz, 3H). Mass (m/z): 342 [M+H]⁺.

### Compound 596

### 7-((4-(4-methylpiperidin-1-yl)phenyl)amino)-2H-pyrido[3,2-b][1,4]oxazin-3(4H)-one

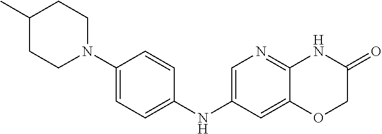 [SMILES: CC1CCN(c2ccc(Nc3cnc4c(c3)OCC(=O)N4)cc2)CC1]

The titled compound 596 (22.5 mg, 15.19%) as a green solid was prepared according to the procedure outlined for compound 1. ¹H NMR (400 MHz, DMSO-d₆) δ 10.93 (s, 1H), 7.77 (s, 1H), 7.54 (d, J=2.4 Hz, 1H), 6.90 (d, J=8.8 Hz, 2H), 6.86-6.83 (m, 3H), 4.53 (s, 2H), 3.47 (d, J=12.4 Hz, 2H), 2.58-2.48 (m, 2H), 1.65 (d, J=12.8 Hz, 2H), 1.45-1.38 (m, 1H), 1.20 (dd, J=11.6, 3.2 Hz, 2H), 0.90 (d, J=6.4 Hz, 3H). Mass (m/z): 339 [M+H]⁺.

### Compound 597

### 7-((4-(3,3-difluoropyrrolidin-1-yl)-2-methylphenyl)amino)-2H-benzo[b][1,4]oxazin-3(4H)-one

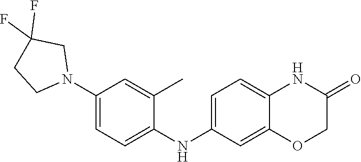 [SMILES: Cc1cc(N2CCC(F)(F)C2)ccc1Nc1ccc2c(c1)OCC(=O)N2]

The titled compound 597 (53.4 mg, 24.23%) as a grey solid was prepared according to the procedure outlined for compound 1. ¹H NMR (400 MHz, DMSO-d₆) δ 10.35 (s, 1H), 7.06 (s, 1H), 6.96 (d, J=8.4 Hz, 1H), 6.63 (d, J=8.4 Hz, 1H), 6.53 (d, J=2.4 Hz, 1H), 6.45 (dd, J=8.4, 2.8 Hz, 1H), 6.22-6.15 (m, 2H), 4.43 (s, 2H), 3.65 (t, J=13.6 Hz, 2H), 3.43 (t, J=7.2 Hz, 2H), 2.54 (d, J=7.6 Hz, 1H), 2.48 (s, 1H), 2.11 (s, 3H). Mass (m/z): 360 [M+H]⁺.

### Compound 598

### 3-amino-6-((2-methyl-4-(4-(trifluoromethyl)piperidin-1-yl)phenyl)amino)-3,4-dihydroquinolin-2(1H)-one

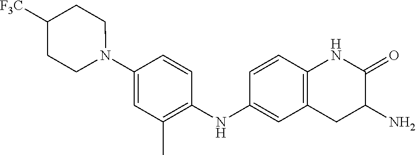 [SMILES: Cc1cc(N2CCC(C(F)(F)F)CC2)ccc1Nc1ccc2c(c1)CC(N)C(=O)N2]

The titled compound 598 (4.3 mg, 8.56%) as a white solid was prepared according to the procedure outlined for compound 1. ¹H NMR (400 MHz, DMSO-d₆) δ 10.13 (s, 1H), 6.99 (s, 1H), 6.92 (d, J=8.8 Hz, 1H), 6.80 (d, J=2.8 Hz, 1H), 6.70 (dd, J=8.8, 2.8 Hz, 1H), 6.63 (d, J=8.4 Hz, 1H), 6.53-6.47 (m, 2H), 5.96 (s, 1H), 3.73 (dd, J=13.6, 6.6 Hz, 1H), 3.64 (d, J=12.4 Hz, 2H), 2.88-2.72 (m, 2H), 2.64-2.57 (m, 2H), 2.45-2.35 (m, 1H), 2.08 (s, 3H), 1.84 (d, J=12.4 Hz, 2H), 1.60-1.45 (m, 2H). Mass (m/z): 418.8 [M+H]⁺.

### Compound 599

### 7-{[4-(3-fluoropyrrolidin-1-yl)-2-methylphenyl]amino}-2,4-dihydro-1,4-benzoxazin-3-one

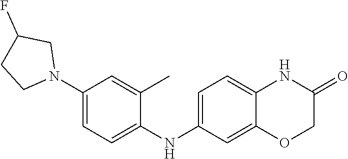 [SMILES: Cc1cc(N2CCC(F)C2)ccc1Nc1ccc2c(c1)OCC(=O)N2]

The titled compound 599 (7 mg, 3.89%) as a white solid was prepared according to the procedure outlined for compound 1. ¹H NMR (400 MHz, DMSO-d₆) δ 10.33 (s, 1H), 7.02 (s, 1H), 6.93 (d, J=8.4 Hz, 1H), 6.62 (d, J=8.4 Hz, 1H), 6.47 (d, J=2.4 Hz, 1H), 6.39 (dd, J=8.4, 2.4 Hz, 1H), 6.21-6.10 (m, 2H), 4.43 (s, 2H), 3.49 (m, 3H), 3.29 (d, J=9.2 Hz, 2H), 2.29-2.13 (m, 2H), 2.08 (d, J=8.0 Hz, 3H). Mass (m/z): 341.9 [M+H]⁺.

### Compound 600

### N-(3-oxo-7-((4-(4-(trifluoromethyl)piperidin-1-yl)phenyl)amino)-2,3-dihydro-4H-benzo[b][1,4]oxazin-4-yl)pivalamide

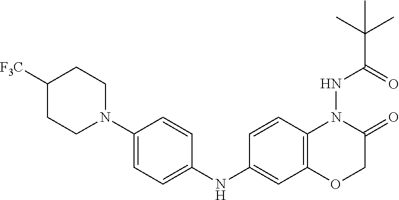 [SMILES: CC(C)(C)C(=O)NN1C(=O)COc2cc(Nc3ccc(N4CCC(C(F)(F)F)CC4)cc3)ccc21]

The titled compound 600 (9.6 mg, 4.3%) as a white solid was prepared according to the procedure outlined for compound 1. ¹H NMR (400 MHz, DMSO-d₆) δ 10.19 (d, J=2.8 Hz, 1H), 7.83 (s, 1H), 6.96 (d, J=9.6 Hz, 2H), 6.89 (d, J=8.4 Hz, 2H), 6.71 (d, J=9.2 Hz, 1H), 6.63-6.52 (m, 2H), 4.77-4.65 (m, 2H), 3.62 (d, J=12.0 Hz, 2H), 2.62 (s, 2H), 2.46-2.38 (m, 1H), 1.88 (d, J=12.8 Hz, 2H), 1.57 (q, J=12.8 Hz, 2H), 1.23 (d, J=3.2 Hz, 9H). Mass (m/z): 491.2 [M+H]⁺.

### Compound 601

### (S)-8-(4-(4-(trifluoromethyl)piperidin-1-yl)-2-methylphenylamino)-3-amino-2,3-dihydrobenzo[b][1,4]oxazepin-4(5H)-one

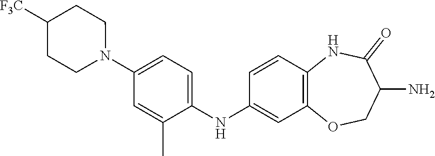 [SMILES: Cc1cc(N2CCC(C(F)(F)F)CC2)ccc1Nc1ccc2c(c1)OCC(N)C(=O)N2]

The titled compound 601 (19.5 mg, 39.8%) as a yellow solid was prepared according to the procedure outlined for compound 1. ¹H NMR (400 MHz, DMSO-d₆) δ 9.48 (s, 1H), 7.24 (s, 1H), 6.99 (d, J=8.8, 1H), 6.85 (s, 1H), 6.82-6.73 (m, 2H), 6.39 (d, J=8.8 Hz, 1H), 6.31 (s, 1H), 4.24-4.15 (m, 1H), 3.92 (t, J=10.8 Hz, 1H), 3.70 (d, J=12.4 Hz, 2H), 3.58 (t, J=9.2 Hz, 1H), 2.65 (t, J=12.4 Hz, 3H), 2.48-2.39 (m, 2H), 2.11 (s, 3H), 1.88 (d, J=12.8 Hz, 2H), 1.72 (s, 2H), 1.64-1.48 (m, 2H). Mass (m/z): 435.2 [M+H]⁺.

### Compound 602

### 2-(hydroxymethyl)-7-((2-methyl-4-(4-(trifluoromethyl)piperidin-1-yl) phenyl)amino)-2H-benzo[b][1,4]oxazin-3(4H)-one

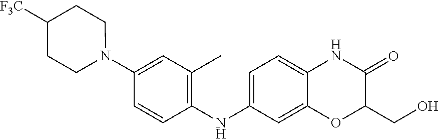 [SMILES: Cc1cc(N2CCC(C(F)(F)F)CC2)ccc1Nc1ccc2c(c1)OC(CO)C(=O)N2]

The titled compound 602 (2.7 mg, 1.54%) as an off-white solid was prepared according to the procedure outlined for compound 1. ¹H NMR (400 MHz, DMSO-d₆) δ 10.35 (s, 1H), 7.05 (s, 1H), 6.97 (d, J=8.8 Hz, 1H), 6.84 (d, J=2.4 Hz, 1H), 6.75 (dd, J=8.8, 2.8 Hz, 1H), 6.61 (d, J=8.4 Hz, 1H), 6.24 (dt, J=6.8, 2.4 Hz, 2H), 5.01 (t, J=5.6 Hz, 1H), 4.49 (dd, J=5.2, 3.2 Hz, 1H), 3.79-3.64 (m, 4H), 2.64 (td, J=12.0, 2.0 Hz, 2H), 2.47-2.40 (m, 1H), 2.11 (s, 3H), 1.88 (d, J=11.2 Hz, 2H), 1.61-1.50 (m, 2H). Mass (m/z): 435.8 [M+H]⁺.

### Compound 603

### N-[6-(2-ethylmorpholin-4-yl)-2-methylpyridin-3-yl]-1-methyl-1,3-benzodiazol-5-amine

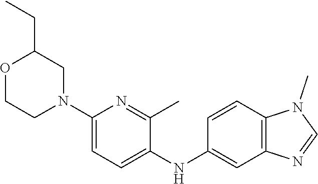 [SMILES: CCC1CN(c2ccc(Nc3ccc4c(c3)ncn4C)c(C)n2)CCO1]

The titled compound 603 (8.7 mg, 4.22%) as a light brown was prepared according to the procedure outlined for compound 1. ¹H NMR (400 MHz, DMSO-d₆) δ 8.31 (s, 1H), 7.42 (d, J=8.8 Hz, 1H), 7.35 (d, J=8.8 Hz, 1H), 7.25 (s, 1H), 6.82 (dd, J=8.8, 1.6 Hz, 1H), 6.68 (dd, J=5.2, 3.2 Hz, 2H), 4.05 (d, J=12.4 Hz, 1H), 4.00-3.91 (m, 2H), 3.81 (s, 3H), 3.55 (td, J=11.6, 2.4 Hz, 1H), 3.40-3.32 (m, 1H), 2.75 (d, J=3.2 Hz, 1H), 2.44 (dd, J=12.4, 10.4 Hz, 1H), 2.24 (s, 3H), 1.57-1.41 (m, 2H), 0.95 (t, J=7.6 Hz, 3H). Mass (m/z): 351.9 [M+H]⁺.

### Compound 604

### 7-((2-methyl-4-(3-methylpyrrolidin-1-yl)phenyl)amino)-2H-benzo[b][1,4]oxazin-3(4H)-one

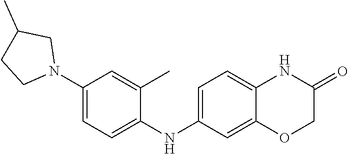 [SMILES: Cc1cc(N2CCC(C)C2)ccc1Nc1ccc2c(c1)OCC(=O)N2]

The titled compound 604 (19.0 mg, 10.35%) as a dark blue solid was prepared according to the procedure outlined for compound 1. ¹H NMR (400 MHz, DMSO-d₆) δ 10.38 (s, 1H), 6.94 (s, 1H), 6.89 (d, J=8.4 Hz, 1H), 6.67 (d, J=8.4 Hz, 1H), 6.39 (d, J=2.4 Hz, 1H), 6.33 (dd, J=8.4, 2.8 Hz, 1H), 6.15 (d, J=2.4 Hz, 1H), 6.11 (dd, J=8.8, 2.4 Hz, 1H), 4.38 (s, 2H), 3.38 (d, J=8.8 Hz, 2H), 3.28-3.20 (m, 2H), 2.80-2.73 (m, 1H), 2.38-2.30 (m, 1H), 2.08 (s, 3H), 1.57 (dd, J=12.0, 8.4 Hz, 1H), 1.08 (d, J=6.8 Hz, 3H). Mass (m/z): 338 [M+H]⁺.

### Compound 605

### N-(6-((2S,6R)-2,6-dimethylmorpholino)-2-methylpyridin-3-yl)-1-methyl-1H-benzo[d] imidazol-5-amine

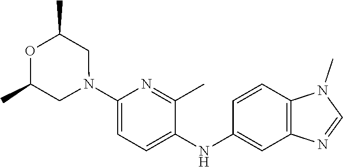 [SMILES: Cc1nc(N2C[C@@H](C)O[C@@H](C)C2)ccc1Nc1ccc2c(c1)ncn2C]

The titled compound 605 (81.3 mg, 51.03%) as an off-white solid was prepared according to the procedure outlined for compound 1. ¹H NMR (400 MHz, DMSO-d₆) δ 7.93 (s, 1H), 7.29 (d, J=8.6 Hz, 2H), 6.98 (s, 1H), 6.73-6.70 (m, 1H), 6.68 (d, J=1.8 Hz, 1H), 6.62 (d, J=8.8 Hz, 1H), 4.01 (dd, J=12.7, 1.9 Hz, 2H), 3.72 (s, 3H), 3.64-3.56 (m, 2H), 2.28 (dd, J=12.6, 10.5 Hz, 2H), 2.22 (s, 3H), 1.14 (s, 3H), 1.12 (s, 3H). Mass (m/z): 352 [M+H]⁺.

### Compound 606

### N-(2-methyl-4-(4-(trifluoromethyl)piperidin-1-yl)phenyl) imidazo[1,2-a]pyridin-7-amine

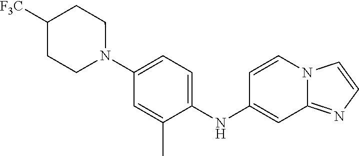 [SMILES: Cc1cc(N2CCC(C(F)(F)F)CC2)ccc1Nc1ccn2ccnc2c1]

The titled compound 606 (13.4 mg, 110%) as a brown solid was prepared according to the procedure outlined for compound 1. ¹H NMR (400 MHz, DMSO-d₆) δ 8.22 (d, J=7.2 Hz, 1H), 7.67 (s, 1H), 7.56 (s, 1H), 7.19 (s, 1H), 7.06 (d, J=8.4 Hz, 1H), 6.92 (s, 1H), 6.83 (d, J=8.8 Hz, 1H), 6.52 (d, J=7.2 Hz, 1H), 6.09 (s, 1H), 3.76 (d, J=12.4 Hz, 2H), 2.72-2.68 (m, 1H), 2.47-2.38 (m, 1H), 2.14 (s, 3H), 1.89 (d, J=12.8 Hz, 2H), 1.64-1.50 (m, 2H). Mass (m/z): 375.5 [M+H]⁺.

### Compound 607

### 2-(aminomethyl)-4-methyl-7-((2-methyl-4-(4-(trifluoromethyl)piperidin-1-yl)phenyl)amino)-2H-benzo[b][1,4]oxazin-3(4H)-one

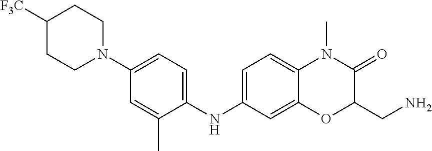 [SMILES: Cc1cc(N2CCC(C(F)(F)F)CC2)ccc1Nc1ccc2c(c1)OC(CN)C(=O)N2C]

The titled compound 607 (4.6 mg, 11%) as an off-white solid was prepared according to the procedure outlined for compound 1. ¹H NMR (400 MHz, DMSO-d₆) δ 7.20 (s, 1H), 6.99 (d, J=9.2 Hz, 1H), 6.92-6.83 (m, 2H), 6.77 (d, J=8.8 Hz, 1H), 6.35 (d, J=8.8 Hz, 1H), 6.29 (s, 1H), 4.43-4.37 (m, 1H), 3.70 (d, J=12.0 Hz, 2H), 3.19 (s, 3H), 2.92-2.88 (m, 1H), 2.64-2.56 (m, 2H), 2.43-2.35 (m, 1H), 2.12 (s, 3H), 1.88 (d, J=12.8 Hz, 2H), 1.63-1.49 (m, 2H). Mass (m/z): 449.5 [M+H]⁺.

### Compound 608

### 8-((2-methyl-4-(4-(trifluoromethyl)piperidin-1-yl)phenyl)amino)-4,5-dihydrobenzo[f][1,4]oxazepin-3(2H)-one

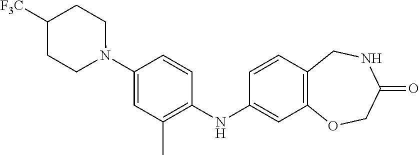 [SMILES: Cc1cc(N2CCC(C(F)(F)F)CC2)ccc1Nc1ccc2c(c1)OCC(=O)NC2]

The titled compound 608 (12.5 mg, 32%) as an off-white solid was prepared according to the procedure outlined for compound 1. ¹H NMR (400 MHz, DMSO-d₆) δ 8.26 (s, 1H), 7.35 (s, 1H), 7.03-6.92 (m, 2H), 6.86 (s, 1H), 6.77 (d, J=8.8 Hz, 1H), 6.30 (d, J=8.4 Hz, 1H), 6.23 (s, 1H), 4.44 (s, 2H), 4.11 (s, 2H), 3.71 (d, J=12.0 Hz, 2H), 2.71-2.61 (m, 2H), 2.47-2.38 (m, 1H), 2.11 (s, 3H), 1.88 (d, J=12.8 Hz, 2H), 1.63-1.48 (m, 2H). Mass (m/z): 420.5 [M+H]⁺.

### Compound 609

### N-(2-methyl-4-(4-(trifluoromethyl)piperidin-1-yl)phenyl)-3-((methylamino) methyl)imidazo[1,2-a]pyridin-7-amine

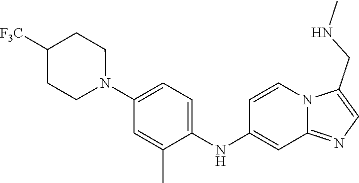 [SMILES: CNCc1cnc2cc(Nc3ccc(N4CCC(C(F)(F)F)CC4)cc3C)ccn12]

The titled compound 609 (16 mg, 24%) as an off-white solid was prepared according to the procedure outlined for compound 1. ¹H NMR (400 MHz, DMSO-d₆) δ 8.13 (d, J=7.6 Hz, 1H), 7.68-7.61 (m, 1H), 7.09-7.02 (m, 2H), 6.91 (s, 1H), 6.83 (d, J=8.8 Hz, 1H), 6.55 (d, J=7.2 Hz, 1H), 6.09 (s, 1H), 3.84 (s, 2H), 3.76 (d, J=12.4 Hz, 2H), 2.72-2.67 (m, 2H), 2.36-2.29 (m, 1H), 2.24 (s, 3H), 2.14 (s, 3H), 1.89 (d, J=12.4 Hz, 2H), 1.64-1.50 (m, 2H). Mass (m/z): 418.5 [M+H]⁺.

### Compound 610

### 8-((4-(3-fluoroazetidin-1-yl)-2-methylphenyl)amino)-2,3-dihydrobenzo[b][1,4]oxazepin-4(5H)-one

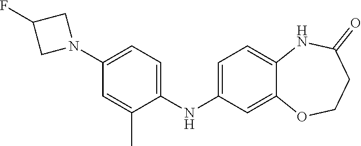 [SMILES: Cc1cc(N2CC(F)C2)ccc1Nc1ccc2c(c1)OCCC(=O)N2]

The titled compound 610 (3.3 mg, 10.6%) as an off-white solid was prepared according to the procedure outlined for compound 1. ¹H NMR (400 MHz, DMSO-d₆) δ 9.31 (s, 1H), 7.15 (s, 1H), 6.96 (d, J=8.4 Hz, 1H), 6.77 (d, J=8.6 Hz, 1H), 6.43-6.25 (m, 3H), 6.18 (s, 1H), 5.64-5.33 (m, 1H), 4.34-4.27 (m, 2H), 4.20-4.05 (m, 2H), 3.88-3.75 (m, 2H), 2.74-2.53 (m, 2H), 2.09 (s, 3H). Mass (m/z): 342.1 [M+H]⁺.

### Compound 611

### (S)-3-amino-8-((4-(3-fluoroazetidin-1-yl)-2-methylphenyl)amino)-2,3-dihydrobenzo[b][1,4]oxazepin-4(5H)-one

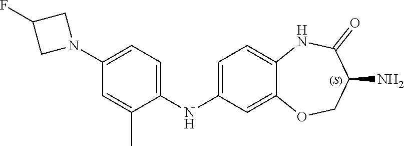

The titled compound 611 (4.4 mg, 11.10%) as an off-white solid was prepared according to the procedure outlined for compound 1. ¹H NMR (400 MHz, DMSO-d₆) δ 9.51 (s, 1H), 7.23 (s, 1H), 6.97 (d, J=8.4 Hz, 1H), 6.77 (d, J=8.8 Hz, 1H), 6.43-6.20 (m, 4H), 5.57-5.35 (m, 1H), 4.30-4.07 (m, 3H), 4.00-3.75 (m, 3H), 3.68-3.60 (m, 1H), 2.81-2.58 (m, 2H), 2.09 (s, 3H). Mass (m/z): 357.1 [M+H]⁺.

### Compound 612

### N-(2-methyl-4-(4-(trifluoromethyl)piperidin-1-yl)phenyl) pyrazolo[1,5-a]pyridin-5-amine

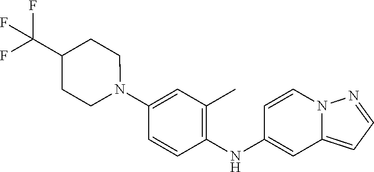 [SMILES: Cc1cc(N2CCC(C(F)(F)F)CC2)ccc1Nc1ccn2nccc2c1]

The titled compound 612 (14.5 mg, 12.5%) as a grey solid was prepared according to the procedure outlined for compound 1. ¹H NMR (400 MHz, DMSO-d₆) δ 8.35 (d, J=7.6 Hz, 1H), 7.68 (d, J=3.8 Hz, 2H), 7.12-7.02 (m, 1H), 6.91 (s, 1H), 6.82 (d, J=8.7 Hz, 1H), 6.48 (d, J=7.5 Hz, 1H), 6.19 (s, 1H), 6.04 (s, 1H), 3.76 (d, J=12.2 Hz, 2H), 2.71 (d, J=12.5 Hz, 2H), 2.44-2.42 (m, 1H), 2.14 (d, J=2.9 Hz, 3H), 1.89 (d, J=12.6 Hz, 2H), 1.57 (q, J=12.7 Hz, 2H). Mass (m/z): 375.2 [M+H]⁺.

### Compound 613

### 3-amino-8-((2-methyl-4-(3-(trifluoromethyl)pyrrolidin-1-yl)phenyl) amino)-2,3-dihydrobenzo[b][1,4]oxazepin-4(5H)-one

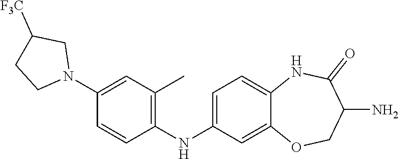 [SMILES: Cc1cc(N2CCC(C(F)(F)F)C2)ccc1Nc1ccc2c(c1)OCC(N)C(=O)N2]

The titled compound 613 (14.6 mg, 44.5%) as a yellow solid was prepared according to the procedure outlined for compound 1. ¹H NMR (400 MHz, DMSO-d₆) δ 9.44 (s, 1H), 7.20 (s, 1H), 6.96 (d, J=8.4 Hz, 1H), 6.75 (d, J=8.4 Hz, 1H), 6.53 (s, 1H), 6.46 (d, J=8.8 Hz, 1H), 6.31 (d, J=8.8 Hz, 1H), 6.22 (s, 1H), 4.18 (t, J=8.8 Hz, 1H), 3.91 (t, J=10.8 Hz, 1H), 3.54 (dt, J=30.8, 8.4 Hz, 2H), 3.28 (t, J=6.8 Hz, 2H), 2.27 (s, 1H), 2.11 (s, 3H), 2.23-2.19 (m, 1H), 1.73 (s, 2H). Mass (m/z): 421.4 [M+H]⁺.

### Compound 614

### 8-((2-methyl-4-(3-(trifluoromethoxy)azetidin-1-yl)phenyl)amino)-2,3-dihydrobenzo[b][1,4]oxazepin-4(5H)-one

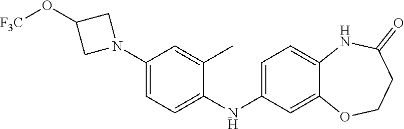 [SMILES: Cc1cc(N2CC(OC(F)(F)F)C2)ccc1Nc1ccc2c(c1)OCCC(=O)N2]

The titled compound 614 (3.0 mg, 9.7%) as a pink solid was prepared according to the procedure outlined for compound 1. ¹H NMR (400 MHz, DMSO-d₆) δ 9.31 (s, 1H), 7.16 (s, 1H), 6.96 (d, J=8.4 Hz, 1H), 6.77 (d, J=8.8 Hz, 1H), 6.40 (s, 1H), 6.32 (t, J=9.6 Hz, 2H), 6.19 (s, 1H), 5.27 (s, 1H), 4.30 (d, J=7.6 Hz, 2H), 4.18 (d, J=7.6 Hz, 2H), 3.84 (s, 2H), 2.09 (s, 3H). Mass (m/z): 408.2 [M+H]⁺.

### Compound 615

### 6-methyl-8-(4-(trifluoromethyl)piperidin-1-yl)pyrazolo[4,3-b]carbazole-1(5H)-carboxamide

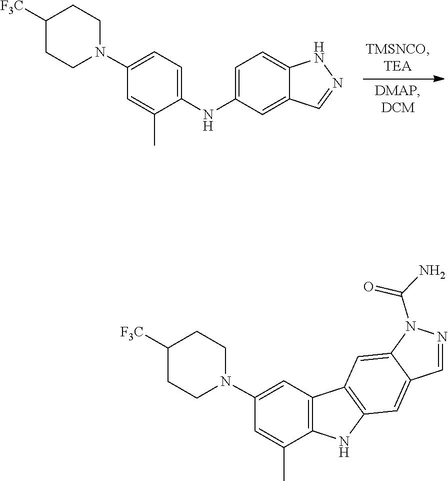 [SMILES: Cc1cc(N2CCC(C(F)(F)F)CC2)ccc1Nc1ccc2[nH]ncc2c1 ; Cc1cc(N2CCC(C(F)(F)F)CC2)cc2c1[nH]c1cc3cnn(C(N)=O)c3cc12]

To a solution of N-(2-methyl-4-(4-(trifluoromethyl)piperidin-1-yl)phenyl)-1H-indazol-5-amine (200 mg, 0.53 mmol) in DCM (5 mL) was added TEA (108 mg, 1.06 mmol), DMAP (65 mg, 0.53 mmol) and TMSNCO (61 mg, 0.53 mmol) at room temperature. The reaction mixture was heated to 25° C. for 8 h under nitrogen atmosphere. After the reaction was completed, the filtrate was concentrated. The residue was purified by silica gel flash column chromatography (PE/EtOAc=10/1) to afford 6-methyl-8-(4-(trifluoromethyl)piperidin-1-yl)pyrazolo[4,3-b]carbazole-1(5H)-carboxa mide (10.6 mg, 4.8% yield) as a yellow solid. ¹H NMR (400 MHz, CD₃OD) δ 8.12 (s, 1H), 7.53-7.48 (m, 3H), 7.23(d, J=7.6 Hz, 1H), 6.79 (s, 1H), 3.92-3.87 (m, 2H), 3.51-3.40 (m, 1H), 2.70-2.54 (m, 1H), 2.32-2.12 (m, 5H), 1.26-1.21 (m, 2H). Mass (m/z): 415.7 [M+H]⁺.

### Compound 616

### 7-((4-(3-methoxy-3-(trifluoromethyl)pyrrolidin-1-yl)-2-methylphenyl) amino)-2H-benzo[b][1,4]oxazin-3(4H)-one

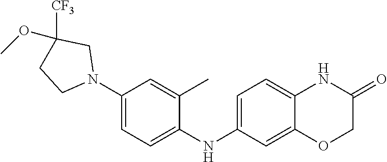 [SMILES: COC1(C(F)(F)F)CCN(c2ccc(Nc3ccc4c(c3)OCC(=O)N4)c(C)c2)C1]

The titled compound 616 (5.2 mg, 2.51%) as a light purple solid was prepared according to the procedure outlined for compound 1. ¹H NMR (400 MHz, DMSO-d₆) δ 10.36 (s, 1H), 7.06 (s, 1H), 6.94 (d, J=8.4 Hz, 1H), 6.63 (d, J=8.4 Hz, 1H), 6.54 (d, J=2.8 Hz, 1H), 6.46 (dd, J=8.8, 2.8 Hz, 1H), 6.22-6.12 (m, 2H), 4.43 (s, 2H), 3.57 (d, J=11.6 Hz, 1H), 3.49 (d, J=11.2 Hz, 1H), 3.45-3.36 (m, 5H), 2.43-2.35 (m, 1H), 2.31 (dd, J=10.8, 5.2 Hz, 1H), 2.09 (d, J=9.6 Hz, 3H). Mass (m/z): 421.8 [M+H]⁺.

### Compound 617

### 7-((4-(3-hydroxy-3-(trifluoromethyl)pyrrolidin-1-yl)-2-methylphenyl) amino)-2H-benzo[b][1,4]oxazin-3(4H)-one

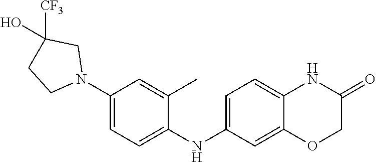 [SMILES: Cc1cc(N2CCC(O)(C(F)(F)F)C2)ccc1Nc1ccc2c(c1)OCC(=O)N2]

The titled compound 617 (44.5 mg, 18.05%) as a dark grey solid was prepared according to the procedure outlined for compound 1. ¹H NMR (400 MHz, DMSO-d₆) δ 10.35 (s, 1H), 7.04 (s, 1H), 6.94 (d, J=8.4 Hz, 1H), 6.62 (d, J=8.4 Hz, 1H), 6.48 (d, J=2.8 Hz, 1H), 6.44-6.37 (m, 2H), 6.20-6.12 (m, 2H), 4.43 (s, 2H), 3.59 (d, J=10.8 Hz, 1H), 3.48-3.36 (m, 2H), 3.29 (d, J=10.8 Hz, 1H), 2.29-2.22 (m, 1H), 2.13-2.06 (m, 4H). Mass (m/z): 407.8 [M+H]⁺.

### Compound 618

### 7-{[4-(3-methoxypyrrolidin-1-yl)-2-methylphenyl]amino}-2,4-dihydro-1,4-benzoxazin-3-one

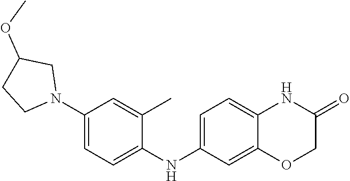 [SMILES: COC1CCN(c2ccc(Nc3ccc4c(c3)OCC(=O)N4)c(C)c2)C1]

The titled compound 618 (3.6 mg, 1.6%) as a light brown solid was prepared according to the procedure outlined for compound 1. ¹H NMR (400 MHz, DMSO-d₆) δ 10.33 (s, 1H), 7.01 (s, 1H), 6.91 (d, J=8.4 Hz, 1H), 6.61 (d, J=8.4 Hz, 1H), 6.43 (d, J=2.4 Hz, 1H), 6.36 (dd, J=8.4, 2.4 Hz, 1H), 6.20-6.08 (m, 2H), 4.42 (s, 2H), 3.39 (dd, J=10.8, 5.2 Hz, 2H), 3.26 (s, 3H), 3.22 (dd, J=16.0, 8.8 Hz, 3H), 2.09 (s, 3H), 2.05 (dd, J=10.0, 5.2 Hz, 2H). Mass (m/z): 353.9 [M+H]⁺.

### Compound 619

### 5-fluoro-7-({4-[4-(trifluoromethyl)piperidin-1-yl]phenyl}amino)-2,4-dihydro-1,4-benzoxazin-3-one

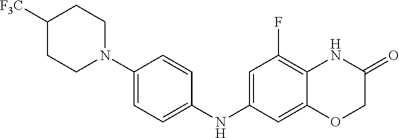 [SMILES: O=C1COc2cc(Nc3ccc(N4CCC(C(F)(F)F)CC4)cc3)cc(F)c2N1]

The titled compound 619 (5.1 mg, 3.08%) as a light brown solid was prepared according to the procedure outlined for compound 1. ¹H NMR (400 MHz, DMSO-d₆) δ 10.60 (s, 1H), 7.92 (s, 1H), 6.94 (dd, J=21.2, 9.2 Hz, 4H), 6.43-6.23 (m, 2H), 4.51 (s, 2H), 3.65 (d, J=12.4 Hz, 2H), 2.63 (dd, J=12.4, 10.4 Hz, 2H), 2.47-2.38 (m, 1H), 1.88 (d, J=12.4 Hz, 2H), 1.64-1.49 (m, 2H). Mass (m/z): 409.9 [M+H]⁺.

### Compound 620

### 3-(2-methoxyethyl)-1-methyl-5-((2-methyl-4-(3-(trifluoromethyl) pyrrolidin-1-yl)phenyl)amino)-1,3-dihydro-2H-benzo[d]imidazol-2-one

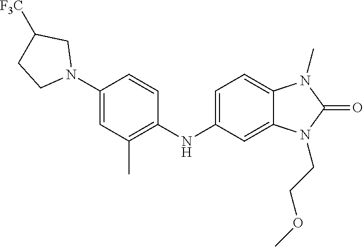 [SMILES: COCCn1c(=O)n(C)c2ccc(Nc3ccc(N4CCC(C(F)(F)F)C4)cc3C)cc21]

The titled compound 620 (79.2 mg, 28.7%) as a light purple solid was prepared according to the procedure outlined for compound 1. ¹H NMR (400 MHz, DMSO-d₆) δ 6.97 (d, J=8.0 Hz, 1H), 6.91 (s, 1H), 6.86 (d, J=8.0 Hz, 1H), 6.53 (s, 1H), 6.48 (s, 1H), 6.44 (d, J=8.4 Hz, 1H), 6.35 (d, J=8.0 Hz, 1H), 3.84 (s, 2H), 3.55-3.44 (m, 3H), 3.33 (s, 2H), 3.29 (s, 2H), 3.24 (s, 3H), 3.20 (s, 3H), 2.26 (s, 1H), 2.13 (s, 3H), 2.07 (s, 1H). Mass (m/z): 449 [M+H]⁺.

### Compound 621

### 7-((2-methyl-4-(3-(trifluoromethoxy)azetidin-1-yl)phenyl)amino)-2H-benzo[b][1,4]oxazin-3(4H)-one

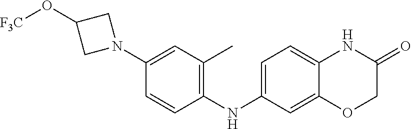 [SMILES: Cc1cc(N2CC(OC(F)(F)F)C2)ccc1Nc1ccc2c(c1)OCC(=O)N2]

The titled compound 621 (4.6 mg, 5.8%) as a grey solid was prepared according to the procedure outlined for compound 1. ¹H NMR (400 MHz, DMSO-d₆) δ 10.35 (s, 1H), 7.06 (s, 1H), 6.94 (d, J=8.4 Hz, 1H), 6.64 (d, J=8.4 Hz, 1H), 6.39 (s, 1H), 6.32 (d, J=8.4 Hz, 1H), 6.25-6.11 (m, 2H), 5.27 (s, 1H), 4.43 (d, J=3.2 Hz, 2H), 4.18 (t, J=7.6 Hz, 2H), 3.81 (d, J=8.4 Hz, 2H), 2.09 (d, J=3.2 Hz, 3H). Mass (m/z): 394.3 [M+H]⁺.

### Compound 622

### 8-((4-(3,3-difluoropyrrolidin-1-yl)-2-methylphenyl)amino)-2,3-dihydrobenzo[b][1,4]oxazepin-4(5H)-one

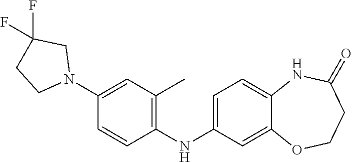 [SMILES: Cc1cc(N2CCC(F)(F)C2)ccc1Nc1ccc2c(c1)OCCC(=O)N2]

The titled compound 622 (10.1 mg, 26.8%) as a pink solid was prepared according to the procedure outlined for compound 1. ¹H NMR (400 MHz, DMSO-d₆) δ 9.30 (s, 1H), 7.16 (s, 1H), 6.98 (d, J=8.4 Hz, 1H), 6.77 (d, J=8.4 Hz, 1H), 6.53 (s, 1H), 6.46 (d, J=8.8 Hz, 1H), 6.29 (d, J=8.8 Hz, 1H), 6.18 (s, 1H), 4.30 (d, J=7.2 Hz, 2H), 3.65 (t, J=13.6 Hz, 2H), 3.43 (d, J=7.6 Hz, 2H), 2.61-2.56 (m, 4H), 2.11 (s, 3H). Mass (m/z): 374.4 [M+H]⁺.

### Compound 623

### 8-((2-methyl-4-(3-(trifluoromethoxy)azetidin-1-yl)phenyl)amino)-2,3-dihydrobenzo[b][1,4]oxazepin-4(5H)-one

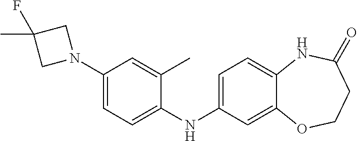 [SMILES: Cc1cc(N2CC(C)(F)C2)ccc1Nc1ccc2c(c1)OCCC(=O)N2]

The titled compound 623 (4.8 mg, 16.1%) as a red solid was prepared according to the procedure outlined for compound 1. ¹H NMR (400 MHz, DMSO-d₆) δ 9.31 (s, 1H), 7.14 (s, 1H), 6.95 (d, J=9.6 Hz, 1H), 6.83-6.71 (m, 1H), 6.38 (s, 1H), 6.31 (d, J=7.2 Hz, 2H), 6.18 (s, 1H), 4.31 (q, J=5.2 Hz, 2H), 3.87 (dd, J=19.2, 3.2 Hz, 4H), 2.09 (d, J=3.2 Hz, 3H), 1.72-1.52 (m, 3H). Mass (m/z): 356.4 [M+H]⁺.

### Compound 624

### N-(2-methyl-4-(4-(trifluoromethyl)piperidin-1-yl)phenyl) imidazo[1,2-a]pyridin-6-amine

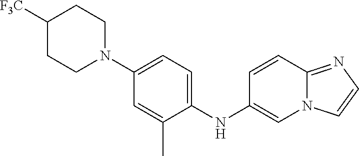 [SMILES: Cc1cc(N2CCC(C(F)(F)F)CC2)ccc1Nc1ccc2nccn2c1]

The titled compound 624 (18.8 mg, 15%) as a grey solid was prepared according to the procedure outlined for compound 1. ¹H NMR (400 MHz, DMSO-d₆) δ 7.75 (s, 1H), 7.58 (d, J=2.0 Hz, 1H), 7.44-7.34 (m, 2H), 7.03 (s, 1H), 7.02-6.94 (m, 2H), 6.88 (d, J=2.8 Hz, 1H), 6.81-6.74 (m, 1H), 3.70 (d, J=12.4 Hz, 2H), 2.71-2.60 (m, 2H), 2.48-2.43 (m, 1H), 2.17 (s, 3H), 1.93-1.84 (m, 2H), 1.64-1.49 (m, 2H). Mass (m/z): 375.5 [M+H]⁺.

### Compound 625

### 5-((2-methyl-4-(4-(trifluoromethyl)piperidin-1-yl)phenyl)amino) pyrazolo[1,5-a]pyridine-3-carboxamide

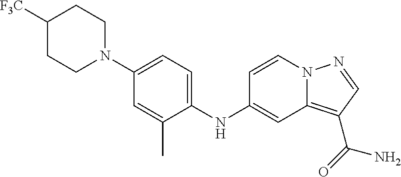 [SMILES: Cc1cc(N2CCC(C(F)(F)F)CC2)ccc1Nc1ccn2ncc(C(N)=O)c2c1]

The titled compound 625 (16.1 mg, 34%) as an off-white solid was prepared according to the procedure outlined for compound 1. ¹H NMR (400 MHz, DMSO-d₆) δ 8.39 (d, J=7.6 Hz, 1H), 8.24 (s, 1H), 8.04 (s, 1H), 7.32-7.28 (m, 1H), 7.08 (d, J=8.8 Hz, 1H), 6.94 (dd, J=11.2, 2.8 Hz, 2H), 6.84 (dd, J=8.8, 2.8 Hz, 1H), 6.67-6.62 (m, 1H), 6.56 (dd, J=7.6, 2.4 Hz, 1H), 3.79 (d, J=12.4 Hz, 2H), 2.77-2.66 (m, 2H), 2.45-2.38 (m, 1H), 2.14 (s, 3H), 1.89 (d, J=12.4 Hz, 2H), 1.65-1.50 (m, 2H). Mass (m/z): 420.5 [M+H]⁺.

### Compound 626

### 8-((4-(3,3-difluoroazetidin-1-yl)-2-methylphenyl)amino)-2,3-dihydrobenzo[b][1,4]oxazepin-4(5H)-one

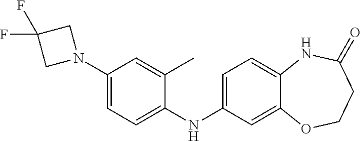 [SMILES: Cc1cc(N2CC(F)(F)C2)ccc1Nc1ccc2c(c1)OCCC(=O)N2]

The titled compound 626 (10.5 mg, 28.1%) as a red solid was prepared according to the procedure outlined for compound 1. ¹H NMR (400 MHz, DMSO-d₆) δ 9.33 (s, 1H), 7.19 (s, 1H), 7.00 (d, J=8.4 Hz, 1H), 6.78 (d, J=8.4 Hz, 1H), 6.47 (d, J=2.8 Hz, 1H), 6.39 (dd, J=8.4, 2.8 Hz, 1H), 6.33 (dd, J=8.4, 2.8 Hz, 1H), 6.21 (d, J=2.4 Hz, 1H), 4.31 (t, J=6.0 Hz, 2H), 4.22 (t, J=12.4 Hz, 4H), 2.57 (t, J=6.0 Hz, 2H), 2.11 (s, 3H). Mass (m/z): 360.4 [M+H]⁺.

### Compound 627

### 3-amino-8-((4-(3,3-difluoroazetidin-1-yl)-2-methylphenyl)amino)-2,3-dihydrobenzo[b][1,4]oxazepin-4(5H)-one

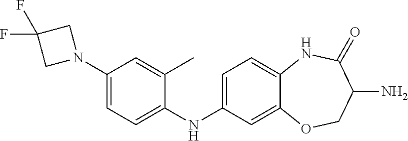 [SMILES: Cc1cc(N2CC(F)(F)C2)ccc1Nc1ccc2c(c1)OCC(N)C(=O)N2]

The titled compound 627 (8.1 mg, 32.3%) as a yellow solid was prepared according to the procedure outlined for compound 1. ¹H NMR (400 MHz, DMSO-d₆) δ 9.47 (s, 1H), 7.26 (s, 1H), 7.01 (d, J=8.4 Hz, 1H), 6.77 (d, J=8.4 Hz, 1H), 6.48 (d, J=2.8 Hz, 1H), 6.38 (ddd, J=18.0, 8.4, 2.8 Hz, 2H), 6.27 (d, J=2.4 Hz, 1H), 4.32-4.16 (m, 4H), 3.92 (t, J=10.8 Hz, 1H), 3.58 (dd, J=11.2, 6.4 Hz, 1H), 2.11 (s, 3H), 1.79 (s, 1H). Mass (m/z): 375.2 [M+H]⁺.

### Compound 628

### 8-((2-methyl-4-(3-(trifluoromethyl)pyrrolidin-1-yl)phenyl)amino)-2,3-dihydrobenzo[b][1,4]oxazepin-4(5H)-one

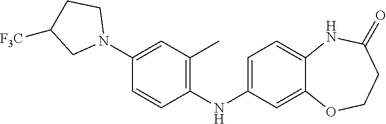 [SMILES: Cc1cc(N2CCC(C(F)(F)F)C2)ccc1Nc1ccc2c(c1)OCCC(=O)N2]

The titled compound 628 (22.3 mg, 34.4%) as a grey solid was prepared according to the procedure outlined for compound 1. ¹H NMR (400 MHz, DMSO-d₆) δ 9.30 (s, 1H), 7.13 (s, 1H), 6.95 (d, J=8.4 Hz, 1H), 6.76 (d, J=8.8 Hz, 1H), 6.53 (d, J=2.8 Hz, 1H), 6.45 (dd, J=8.8, 2.8 Hz, 1H), 6.28 (dd, J=8.8, 2.6 Hz, 1H), 6.16 (d, J=2.4 Hz, 1H), 4.31 (t, J=6.0 Hz, 2H), 3.50 (t, J=8.8 Hz, 1H), 3.41-3.34 (m, 2H), 3.31-3.23 (m, 2H), 2.56 (t, J=6.0 Hz, 2H), 2.31-2.21 (m, 1H), 2.10 (s, 3H), 2.09-2.02 (m, 1H). Mass (m/z): 406.2 [M+H]⁺.

### Compound 629

### 8-((4-(4,4-difluoropiperidin-1-yl)-2-methylphenyl)amino)-2,3-dihydrobenzo[b][1,4]oxazepin-4(5H)-one

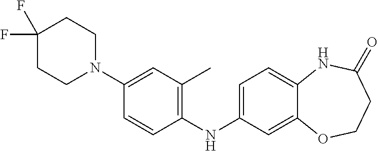 [SMILES: Cc1cc(N2CCC(F)(F)CC2)ccc1Nc1ccc2c(c1)OCCC(=O)N2]

The titled compound 629 (17 mg, 20.6%) as a grey solid was prepared according to the procedure outlined for compound 1. ¹H NMR (400 MHz, DMSO-d₆) δ 9.34 (s, 1H), 7.19 (s, 1H), 6.99 (d, J=8.8 Hz, 1H), 6.89 (d, J=2.8 Hz, 1H), 6.80 (dd, J=8.8, 3.6 Hz, 2H), 6.37 (dd, J=8.8, 2.4 Hz, 1H), 6.27 (d, J=2.4 Hz, 1H), 4.32 (t, J=6.0 Hz, 2H), 3.29-3.19 (m, 4H), 2.57 (t, J=6.0 Hz, 2H), 2.12 (s, 3H), 2.10-2.00 (m, 4H). Mass (m/z): 388.2 [M+H]⁺.

### Compound 630

### 6-((2-methyl-4-(4-(trifluoromethyl)piperidin-1-yl)phenyl)amino) imidazo[1,2-a]pyridine-3-carboxamide

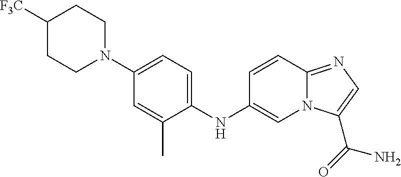 [SMILES: Cc1cc(N2CCC(C(F)(F)F)CC2)ccc1Nc1ccc2ncc(C(N)=O)n2c1]

The titled compound 630 (10 mg, 21%) as an grey solid was prepared according to the procedure outlined for compound 1. ¹H NMR (400 MHz, DMSO-d₆) δ 8.92 (d, J=2.0 Hz, 1H), 8.32 (s, 1H), 8.03-7.83 (m, 1H), 7.66 (d, J=9.6 Hz, 1H), 7.60 (s, 1H), 7.48-7.27 (m, 2H), 7.02 (d, J=8.8 Hz, 1H), 6.98-6.87 (m, 1H), 6.81 (dd, J=8.8, 2.8 Hz, 1H), 3.74 (d, J=12.4 Hz, 2H), 2.71 (t, J=11.6 Hz, 2H), 2.46-2.37 (m, 1H), 2.17 (s, 3H), 1.89 (d, J=11.6 Hz, 2H), 1.67-1.48 (m, 2H). Mass (m/z): 420.5 [M+H]⁺.

### Compound 631

### 4-methyl-8-((2-methyl-4-(4-(trifluoromethyl)piperidin-1-yl)phenyl) amino)-4,5-dihydrobenzo[f][1,4]oxazepin-3(2H)-one

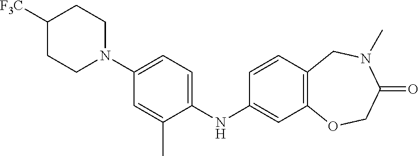 [SMILES: Cc1cc(N2CCC(C(F)(F)F)CC2)ccc1Nc1ccc2c(c1)OCC(=O)N(C)C2]

The titled compound 631 (56 mg, 33%) as an off-white solid was prepared according to the procedure outlined for compound 1. ¹H NMR (400 MHz, DMSO-d₆) δ 7.33 (s, 1H), 7.01-6.90 (m, 2H), 6.86 (d, J=2.8 Hz, 1H), 6.77 (dd, J=8.8, 2.8 Hz, 1H), 6.25 (dd, J=8.2, 2.3 Hz, 1H), 6.13 (d, J=2.4 Hz, 1H), 4.61 (s, 2H), 4.45 (s, 2H), 3.71 (d, J=12.0 Hz, 2H), 2.95 (s, 3H), 2.72-2.60 (m, 2H), 2.47-2.43 (m, 1H), 2.10 (s, 3H), 1.88 (d, J=12.0 Hz, 2H), 1.63-1.48 (m, 2H). Mass (m/z): 434.5 [M+H]⁺.

### Compound 632

### 7-({4-[3-hydroxy-3-(trifluoromethyl)azetidin-1-yl]-2-methylphenyl}amino)-2,4-dihydro-1,4-benzoxazin-3-one

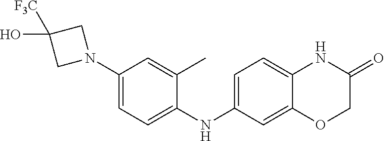 [SMILES: Cc1cc(N2CC(O)(C(F)(F)F)C2)ccc1Nc1ccc2c(c1)OCC(=O)N2]

The titled compound 632 (1.6 mg, 0.64%) as a light brown solid was prepared according to the procedure outlined for compound 1. ¹H NMR (400 MHz, DMSO-d₆) δ 10.37 (s, 1H), 7.25 (s, 1H), 7.07 (s, 1H), 6.95 (d, J=8.4 Hz, 1H), 6.64 (d, J=8.4 Hz, 1H), 6.41 (d, J=2.4 Hz, 1H), 6.33 (dd, J=8.4, 2.4 Hz, 1H), 6.21 (dd, J=8.4, 2.4 Hz, 1H), 6.17 (d, J=2.0 Hz, 1H), 4.44 (s, 2H), 4.10 (d, J=8.8 Hz, 2H), 3.76 (d, J=8.8 Hz, 2H), 2.09 (s, 3H). Mass (m/z): 393.8 [M+H]⁺.

### Compound 633

### 7-({4-[3-methoxy-3-(trifluoromethyl)azetidin-1-yl]-2-methylphenyl}amino)-2,4-dihydro-1,4-benzoxazin-3-one

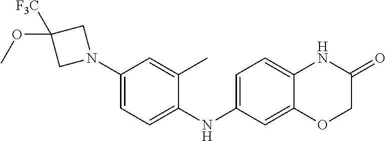 [SMILES: COC1(C(F)(F)F)CN(c2ccc(Nc3ccc4c(c3)OCC(=O)N4)c(C)c2)C1]

The titled compound 633 (4.0 mg, 1.82%) as a light brown solid was prepared according to the procedure outlined for compound 1. ¹H NMR (400 MHz, DMSO-d₆) δ 10.36 (s, 1H), 7.08 (s, 1H), 6.96 (d, J=8.4 Hz, 1H), 6.64 (d, J=8.4 Hz, 1H), 6.43 (d, J=2.4 Hz, 1H), 6.36 (dd, J=8.4, 2.4 Hz, 1H), 6.24-6.16 (m, 2H), 4.44 (s, 2H), 4.05-3.98 (m, 4H), 3.47 (s, 3H), 2.10 (s, 3H). Mass (m/z): 407.8 [M+H]⁺.

### Compound 634

### 7-((4-(tert-butylamino)-2-methylphenyl)amino)-2H-benzo[b][1,4]oxazin-3(4H)-one

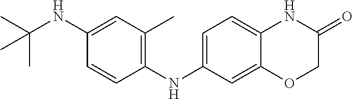 [SMILES: Cc1cc(NC(C)(C)C)ccc1Nc1ccc2c(c1)OCC(=O)N2]

The titled compound 634 (15.4 mg, 10.8%) as a grey solid was prepared according to the procedure outlined for compound 1. ¹H NMR (400 MHz, DMSO-d₆) δ 10.29 (s, 1H), 6.92 (s, 1H), 6.81 (d, J=8.4 Hz, 1H), 6.65-6.59 (m, 2H), 6.55 (dd, J=8.4, 2.8 Hz, 1H), 6.20 (dd, J=8.4, 2.4 Hz, 1H), 6.15 (d, J=2.4 Hz, 1H), 4.66 (s, 1H), 4.43 (s, 2H), 2.04 (s, 3H), 1.26 (s, 9H). Mass (m/z): 326.2 [M+H]⁺.

### Compound 635

### 7-((4-(ethylamino)-2-methylphenyl)amino)-2H-benzo[b][1,4]oxazin-3(4H)-one

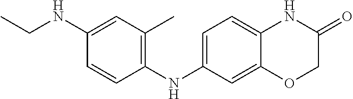 [SMILES: CCNc1ccc(Nc2ccc3c(c2)OCC(=O)N3)c(C)c1]

The titled compound 635 (20.4 mg, 15.6%) as a grey solid was prepared according to the procedure outlined for compound 1. ¹H NMR (400 MHz, DMSO-d₆) δ 10.28 (s, 1H), 6.90 (s, 1H), 6.82 (d, J=8.4 Hz, 1H), 6.61 (d, J=8.4 Hz, 1H), 6.44 (d, J=2.4 Hz, 1H), 6.37 (dd, J=8.4, 2.8 Hz, 1H), 6.20-6.04 (m, 2H), 5.24 (s, 1H), 4.42 (s, 2H), 3.00 (q, J=7.2 Hz, 2H), 2.03 (s, 3H), 1.15 (t, J=7.2 Hz, 3H). Mass (m/z): 298.2 [M+H]⁺.

### Compound 636

### 7-((2-methyl-4-(3,3,4,4-tetrafluoropyrrolidin-1-yl)phenyl)amino)-2H-benzo[b][1,4]oxazin-3(4H)-on

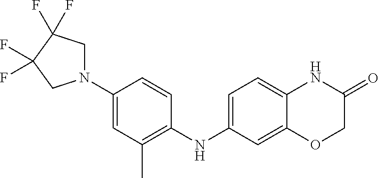 [SMILES: Cc1cc(N2CC(F)(F)C(F)(F)C2)ccc1Nc1ccc2c(c1)OCC(=O)N2]

The titled compound 636 (16.3 mg, 15.1%) as a grey solid was prepared according to the procedure outlined for compound 1. ¹H NMR (400 MHz, DMSO-d₆) δ 10.37 (s, 1H), 7.12 (s, 1H), 7.00 (d, J=8.8 Hz, 1H), 6.70-6.61 (m, 2H), 6.56-6.50 (m, 1H), 6.27-6.17 (m, 2H), 4.44 (s, 2H), 4.03-3.88 (m, 4H), 2.12 (s, 3H). Mass (m/z): 396.2 [M+H]⁺.

### Compound 637

### N-(4-((2S,6R)-2,6-dimethylmorpholino)-2-methylphenyl)-2-methyl-2H-indazol-6-amine

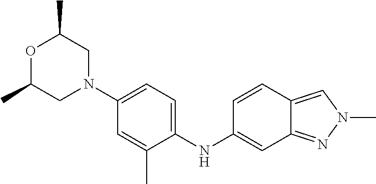

The titled compound 637 (78.8 mg, 50.0%) as a grey solid was prepared according to the procedure outlined for compound 1. ¹H NMR (400 MHz, DMSO-d₆) δ 8.04 (s, 1H), 7.43 (d, J=8.8 Hz, 1H), 7.18 (s, 1H), 7.04 (d, J=8.8 Hz, 1H), 6.86 (d, J=2.8 Hz, 1H), 6.79-6.75 (m, 1H), 6.72-6.68 (m, 1H), 6.36-6.31 (m, 1H), 3.99 (s, 3H), 3.74-3.64 (m, 2H), 3.55-3.50 (m, 2H), 2.27-2.19 (m, 2H), 2.15 (s, 3H), 1.15 (d, J=6.4 Hz, 6H). Mass (m/z): 351.1 [M+H]⁺.

### Compound 638

### N-(4-((2S,6R)-2,6-dimethylmorpholino)-2-methylphenyl)-1-methyl-1H-indazol-6-amine

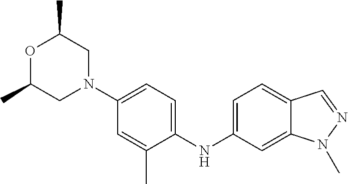 [SMILES: Cc1cc(N2C[C@@H](C)O[C@@H](C)C2)ccc1Nc1ccc2cnn(C)c2c1]

The titled compound 638 (78.5 mg, 49.8%) as a grey solid was prepared according to the procedure outlined for compound 1. ¹H NMR (400 MHz, DMSO-d₆) δ 7.74 (s, 1H), 7.53-7.37 (m, 2H), 7.10 (d, J=8.8 Hz, 1H), 6.88 (d, J=2.8 Hz, 1H), 6.83-6.76 (m, 1H), 6.71-6.64 (m, 1H), 6.37 (s, 1H), 3.77 (s, 3H), 3.73-3.63 (m, 2H), 3.60-3.50 (m, 2H), 2.28-2.20 (m, 2H), 2.16 (s, 3H), 1.16 (d, J=6.4 Hz, 6H). Mass (m/z): 351.2 [M+H]⁺.

### Compound 639

### 7-((4-(3-methoxy-3-methylazetidin-1-yl)-2-methylphenyl) amino)-2H-benzo[b][1,4] oxazin-3(4H)-one

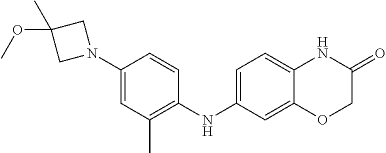 [SMILES: COC1(C)CN(c2ccc(Nc3ccc4c(c3)OCC(=O)N4)c(C)c2)C1]

The titled compound 639 (10.2 mg, 5.65%) as a purple solid was prepared according to the procedure outlined for compound 1. ¹H NMR (400 MHz, DMSO-d₆) δ 10.34 (s, 1H), 7.03 (s, 1H), 6.93 (s, 1H), 6.64 (s, 1H), 6.26-6.28 (s, 1H), 6.19-6.20 (s, 1H), 6.17-6.18 (s, 1H), 6.14-6.15 (s, 1H), 4.43 (m, 2H), 3.62-3.65 (m, 4H), 3.18 (m, 3H), 2.07 (m, 3H), 1.47 (m, 3H). Mass (m/z): 354.2 [M+H]⁺.

### Compound 640

### 7-((4-(3-isopropoxyazetidin-1-yl)-2-methylphenyl)amino)-2H-benzo[b][1,4]oxazin-3(4H)-one

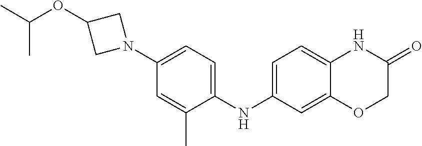 [SMILES: Cc1cc(N2CC(OC(C)C)C2)ccc1Nc1ccc2c(c1)OCC(=O)N2]

The titled compound 640 (7.1 mg, 2.88%) as a yellow solid was prepared according to the procedure outlined for compound 1. ¹H NMR (400 MHz, DMSO-d₆) δ 10.34 (s, 1H), 7.02 (s, 1H), 6.91 (d, J=8.4 Hz, 1H), 6.63 (d, J=8.4 Hz, 1H), 6.33-6.32 (m, 1H), 6.27-6.24 (m, 1H), 6.20-6.17 (m, 1H), 6.15-6.14 (m, 1H), 4.52-4.37 (m, 3H), 4.06 (t, J=7.2 Hz, 2H), 3.66-3.60 (m, 1H), 3.48-3.45 (m, 2H), 2.07 (s, 3H), 1.10 (d, J=6.0 Hz, 6H). Mass (m/z): 368.2 [M+H]⁺.

### Compound 641

### 7-((4-(3-azabicyclo[3.1.0]hexan-3-yl)-2-methylphenyl) amino)-2H-benzo[b][1,4]oxazin-3(4H)-one

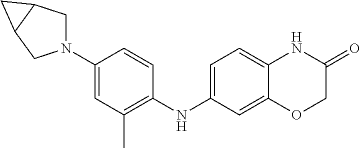 [SMILES: Cc1cc(N2CC3CC3C2)ccc1Nc1ccc2c(c1)OCC(=O)N2]

The titled compound 641 (5 mg, 2%) as a white solid was prepared according to the procedure outlined for compound 1. ¹H NMR (400 MHz, DMSO-d₆) δ 10.34 (s, 1H), 7.01 (s, 1H), 6.89 (d, J=8.4 Hz, 1H), 6.63 (d, J=8.4 Hz, 1H), 6.38 (s, 2H), 6.18-6.11 (m, 2H), 4.42 (s, 2H), 3.46 (d, J=9.2 Hz, 2H), 3.11 (d, J=8.4 Hz, 2H), 2.07 (s, 3H), 1.68-1.63 (m, 2H), 0.68 (td, J=7.6, 4.0 Hz, 1H), 0.27 (d, J=4.0 Hz, 1H). Mass (m/z): 336.2 [M+H]⁺.

### Compound 642

### 7-((4-(2-methyl-4-(trifluoromethyl)piperidin-1-yl)phenyl)amino)-2H-benzo[b][1,4]oxazin-3(4H)-one

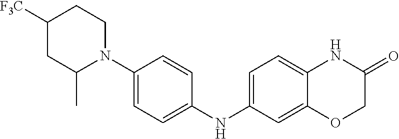 [SMILES: CC1CC(C(F)(F)F)CCN1c1ccc(Nc2ccc3c(c2)OCC(=O)N3)cc1]

The titled compound 642 (6.5 mg, 8.3%) as a purple solid was prepared according to the procedure outlined for compound 1. ¹H NMR (400 MHz, DMSO-d6) δ 10.50 (s, 1H), 7.93 (s, 1H), 7.06-6.87 (m, 4H), 6.76 (d, J=8.4 Hz, 1H), 6.69-6.52 (m, 2H), 4.50 (s, 2H), 3.02 (d, J=11.6 Hz, 1H), 2.84 (t, J=7.6 Hz, 1H), 2.78-2.68 (m, 1H), 1.96-1.76 (m, 2H), 1.56 (tt, J=12.6, 6.4 Hz, 1H), 1.35-1.12 (m, 2H), 0.84 (d, J=6.0 Hz, 3H). Mass (m/z): 406.2 [M+H]⁺.

### Compound 643

### 7-((2-methyl-4-(4-(trifluoromethyl)piperidin-1-yl)phenyl)amino)-1,3,4,5-tetrahydro-2H-benzo[b][1,4]diazepin-2-one

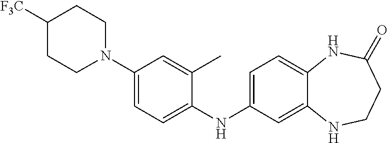 [SMILES: Cc1cc(N2CCC(C(F)(F)F)CC2)ccc1Nc1ccc2c(c1)NCCC(=O)N2]

The titled compound 643 (2.5 mg, 12.3%) as a blue solid was prepared according to the procedure outlined for compound 1. ¹H NMR (400 MHz, DMSO-d6) δ 6.90-6.77 (m, 2H), 6.67 (dd, J=8.8, 3.2 Hz, 1H), 6.61 (d, J=8.0 Hz, 2H), 6.56-6.44 (m, 2H), 4.87 (s, 2H), 3.65-3.53 (m, 3H), 2.97 (t, J=4.4 Hz, 2H), 2.61-2.59 (m, 2H), 2.12 (s, 3H), 1.87 (d, J=12.4 Hz, 2H), 1.61-1.51 (m, 2H). Mass (m/z): 419.2 [M+H]⁺.

### Compound 644

### 7-((2-methyl-4-(4-(trifluoromethyl)piperidin-1-yl)phenyl)amino)-3,4-dihydro-1H-benzo[e][1,4]diazepine-2,5-dione

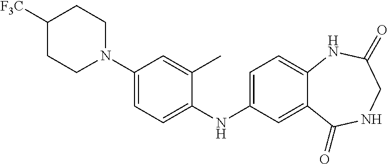 [SMILES: Cc1cc(N2CCC(C(F)(F)F)CC2)ccc1Nc1ccc2c(c1)C(=O)NCC(=O)N2]

The titled compound 644 (12.3 mg, 14.7%) as a yellow solid was prepared according to the procedure outlined for compound 1. ¹H NMR (400 MHz, DMSO-d6) δ 10.12 (s, 1H), 8.48 (t, J=5.6 Hz, 1H), 7.40 (s, 2H), 7.27 (s, 2H), 7.15 (s, 2H), 6.97 (d, J=8.4 Hz, 1H), 3.65 (s, 2H), 3.56 (d, J=5.6 Hz, 4H), 2.21 (s, 3H), 2.00 (d, J=11.2 Hz, 2H). Mass (m/z): 433.2 [M+H]⁺.

### Compound 645

### 8-[4-[3-methoxy-3-(trifluoromethyl)azetidin-1-yl]-2-methyl-anilino]-3,5-dihydro-2H-1,5-benzoxazepin-4-one

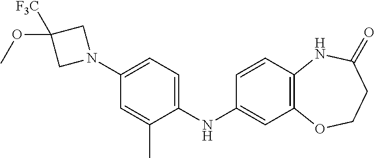 [SMILES: COC1(C(F)(F)F)CN(c2ccc(Nc3ccc4c(c3)OCCC(=O)N4)c(C)c2)C1]

The titled compound 645 (52 mg, 82%) as a light brown solid was prepared according to the procedure outlined for compound 1. ¹H NMR (400 MHz, DMSO-d₆) δ 9.32 (s, 1H), 7.17 (s, 1H), 6.98 (d, J=8.4 Hz, 1H), 6.78 (d, J=8.4 Hz, 1H), 6.44 (d, J=2.8 Hz, 1H), 6.37 (dd, J=8.4, 2.8 Hz, 1H), 6.31 (dd, J=8.8, 2.8 Hz, 1H), 6.20 (d, J=2.4 Hz, 1H), 4.31 (t, J=6.0 Hz, 2H), 4.07-3.97 (m, 4H), 3.48 (s, 3H), 2.60-2.53 (m, 2H), 2.10 (s, 3H). Mass (m/z): 422.4 [M+H]⁺.

### Compound 646

### 3-hydroxy-8-((2-methyl-4-(4-(trifluoromethyl)piperidin-1-yl)phenyl) amino)-2,3-dihydrobenzo[b][1,4]oxazepin-4(5H)-one

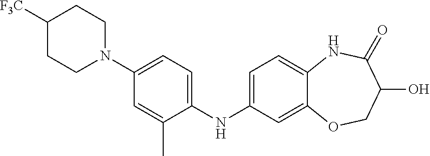 [SMILES: Cc1cc(N2CCC(C(F)(F)F)CC2)ccc1Nc1ccc2c(c1)OCC(O)C(=O)N2]

The titled compound 646 (5.6 mg, 13.12%) as a grey solid was prepared according to the procedure outlined for compound 1. ¹H NMR (400 MHz, DMSO-d₆) δ 10.35 (s, 1H), 7.05 (s, 1H), 6.97 (d, J=8.8 Hz, 1H), 6.84 (d, J=2.8 Hz, 1H), 6.78-6.72 (m, 1H), 6.61 (d, J=8.4 Hz, 1H), 6.29-6.20 (m, 2H), 5.00 (t, J=5.6 Hz, 1H), 4.51-4.47 (m, 1H), 3.81-3.64 (m, 4H), 2.70-2.60 (m, 2H), 2.47-2.38 (m, 1H), 2.11 (s, 3H), 1.92-1.83 (m, 2H), 1.64-1.48 (m, 2H). Mass (m/z): 436.2 [M+H]⁺.

### Compound 647

### 8-((4-(3-methoxy-3-(trifluoromethyl)pyrrolidin-1-yl)-2-methylphenyl) amino)-2,3-dihydrobenzo[b][1,4]oxazepin-4(5H)-one

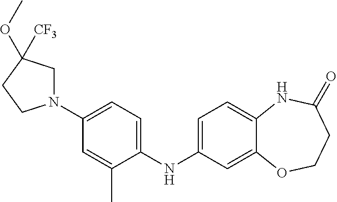 [SMILES: COC1(C(F)(F)F)CCN(c2ccc(Nc3ccc4c(c3)OCCC(=O)N4)c(C)c2)C1]

The titled compound 647 (20 mg, 35.52%) as a grey solid was prepared according to the procedure outlined for compound 1. ¹H NMR (400 MHz, DMSO-d₆) δ 9.30 (s, 1H), 7.14 (s, 1H), 6.96 (d, J=8.4 Hz, 1H), 6.76 (d, J=8.4 Hz, 1H), 6.55 (d, J=2.8 Hz, 1H), 6.50-6.44 (m, 1H), 6.31-6.26 (m, 1H), 6.17 (d, J=2.4 Hz, 1H), 4.31 (t, J=6.0 Hz, 2H), 3.58 (d, J=11.6 Hz, 1H), 3.50 (d, J=11.6 Hz, 1H), 3.46-3.38 (m, 3H), 3.33 (s, 3H), 2.56 (t, J=6.0 Hz, 2H), 2.46-2.36 (m, 1H), 2.35-2.24 (m, 1H), 2.11 (s, 3H). Mass (m/z): 436.1 [M+H]⁺.

### Compound 648

### N-(2-methyl-4-((2S,6R)-2,6-dimethylmorpholino)phenyl)-1H-indazol-6-amine

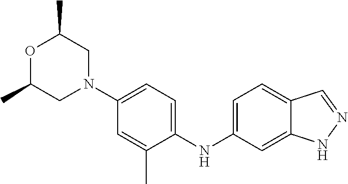 [SMILES: Cc1cc(N2C[C@@H](C)O[C@@H](C)C2)ccc1Nc1ccc2cn[nH]c2c1]

The titled compound 648 (48.5 mg, 60.42%) as a grey solid was prepared according to the procedure outlined for compound 1. ¹H NMR (400 MHz, DMSO-d₆) δ 12.28 (s, 1H), 7.76 (s, 1H), 7.45 (d, J=8.8 Hz, 1H), 7.39 (s, 1H), 7.06 (d, J=8.8 Hz, 1H), 6.88 (d, J=2.8 Hz, 1H), 6.81-6.75 (m, 1H), 6.70-6.63 (m, 1H), 6.36-6.31 (m, 1H), 3.76-3.63 (m, 2H), 3.57-3.49 (m, 2H), 2.28-2.20 (m, 2H), 2.14 (s, 3H), 1.16 (d, J=6.4 Hz, 6H). Mass (m/z): 337.1 [M+H]⁺.

### Compound 649

### 8-[4-[3-hydroxy-3-(trifluoromethyl)azetidin-1-yl]-2-methyl-anilino]-3,5-dihydro-2H-1,5-benzoxazepin-4-one

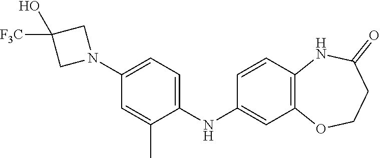 [SMILES: Cc1cc(N2CC(O)(C(F)(F)F)C2)ccc1Nc1ccc2c(c1)OCCC(=O)N2]

The titled compound 649 (3.9 mg, 9.80%) as a pink solid was prepared according to the procedure outlined for compound 1. ¹H NMR (400 MHz, DMSO-d₆) δ 9.31 (s, 1H), 7.23 (s, 1H), 7.15 (s, 1H), 6.97 (d, J=8.4 Hz, 1H), 6.77 (d, J=8.4 Hz, 1H), 6.41 (d, J=2.8 Hz, 1H), 6.38-6.26 (m, 2H), 6.18 (d, J=2.4 Hz, 1H), 4.35-4.27 (m, 2H), 4.10 (d, J=8.8 Hz, 2H), 3.77 (d, J=8.8 Hz, 2H), 2.60-2.52 (m, 2H), 2.09 (s, 3H). Mass (m/z): 408.4 [M+H]⁺.

### Compound 650

### 7-((2-methyl-4-(4-(trifluoromethyl)piperidin-1-yl)phenyl)amino)-1,5-dihydrobenzo[e][1,4]oxazepin-2(3H)-one

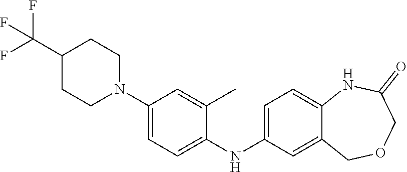 [SMILES: Cc1cc(N2CCC(C(F)(F)F)CC2)ccc1Nc1ccc2c(c1)COCC(=O)N2]

The titled compound 650 (7.2 mg, 39%) as a brown solid was prepared according to the procedure outlined for compound 1. ¹H NMR (400 MHz, DMSO-d6) δ 9.90 (s, 1H), 7.11 (s, 1H), 6.93 (dd, J=24.8, 8.4 Hz, 2H), 6.84 (d, J=2.8 Hz, 1H), 6.74 (dd, J=8.8, 2.8 Hz, 1H), 6.57 (dd, J=8.8, 2.8 Hz, 1H), 6.47 (d, J=2.8 Hz, 1H), 4.54 (s, 2H), 4.31 (s, 2H), 3.68 (d, J=12.0 Hz, 2H), 2.71-2.63 (m, 2H), 2.43-2.41 (m, 1H), 2.11 (s, 3H), 1.88 (d, J=12.0 Hz, 2H), 1.57 (tt, J=12.8, 6.4 Hz, 2H). Mass (m/z): 420.2 [M+H]⁺.

### Compound 651

### 1-[3-methyl-4-[(1-methylbenzimidazol-5-yl)amino]phenyl]-3-(trifluoromethyl)azetidin-3-ol

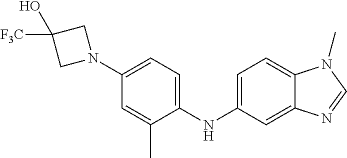 [SMILES: Cc1cc(N2CC(O)(C(F)(F)F)C2)ccc1Nc1ccc2c(c1)ncn2C]

The titled compound 651 (51 mg, 32%) as a yellow solid was prepared according to the procedure outlined for compound 1. ¹H NMR (400 MHz, DMSO-d₆) δ 7.94 (s, 1H), 7.30 (d, J=8.8 Hz, 1H), 7.22 (s, 1H), 6.98 (d, J=8.4 Hz, 1H), 6.91 (s, 1H), 6.76 (dd, J=8.8, 2.0 Hz, 1H), 6.72 (d, J=2.0 Hz, 1H), 6.42 (d, J=2.8 Hz, 1H), 6.34 (dd, J=8.4, 2.8 Hz, 1H), 4.10 (d, J=8.8 Hz, 2H), 3.77 (d, J=8.8 Hz, 2H), 3.74 (s, 3H), 2.12 (s, 3H). Mass (m/z): 377.5 [M+H]⁺.

### Compound 652

### 7-[4-[3-(difluoromethoxy)azetidin-1-yl]-2-methyl-anilino]-4H-1,4-benzoxazin-3-one

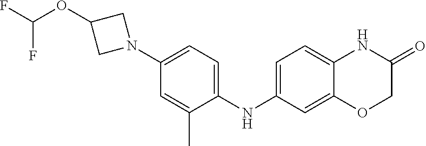 [SMILES: Cc1cc(N2CC(OC(F)F)C2)ccc1Nc1ccc2c(c1)OCC(=O)N2]

The titled compound 652 (23 mg, 14%) as a purple solid was prepared according to the procedure outlined for compound 1. ¹H NMR (400 MHz, DMSO-d₆) δ 10.34 (s, 1H), 7.04 (s, 1H), 6.93 (d, J=8.4 Hz, 1H), 6.77 (t, J=75.2 Hz, 1H), 6.63 (d, J=8.4 Hz, 1H), 6.37 (d, J=2.8 Hz, 1H), 6.29 (dd, J=8.4, 2.8 Hz, 1H), 6.20 (dd, J=8.4, 2.4 Hz, 1H), 6.16 (d, J=2.4 Hz, 1H), 5.09-4.99 (m, 1H), 4.43 (s, 2H), 4.17-4.09 (m, 2H), 3.74-3.66 (m, 2H), 2.08 (s, 3H). Mass (m/z): 376.4 [M+H]⁺.

### Compound 653

### 7-[4-(difluoromethoxy)-2-methyl-anilino]-4H-1,4-benzoxazin-3-one

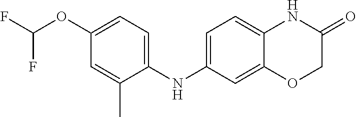 [SMILES: Cc1cc(OC(F)F)ccc1Nc1ccc2c(c1)OCC(=O)N2]

The titled compound 653 (9.0 mg, 9%) as an off-white solid was prepared according to the procedure outlined for compound 1. ¹H NMR (400 MHz, DMSO-d₆) δ 10.48 (s, 1H), 7.27 (s, 1H), 7.09 (d, J=2.0 Hz, 1H), 7.07 (t, J=76 Hz, 1H), 7.02 (d, J=2.8 Hz, 1H), 6.95-6.88 (m, 1H), 6.73 (d, J=8.4 Hz, 1H), 6.51-6.42 (m, 2H), 4.49 (s, 2H), 2.18 (s, 3H). Mass (m/z): 321.4 [M+H]⁺.

### Compound 654

### 7-[4-[4-(difluoromethyl)-1-piperidyl]-2-methyl-anilino]-4H-1,4-benzoxazin-3-one

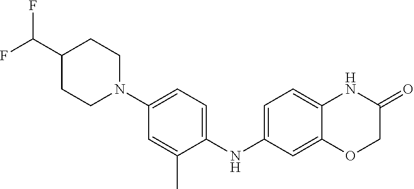 [SMILES: Cc1cc(N2CCC(C(F)F)CC2)ccc1Nc1ccc2c(c1)OCC(=O)N2]

The titled compound 654 (25 mg, 18%) as a brown solid was prepared according to the procedure outlined for compound 1. ¹H NMR (400 MHz, DMSO-d₆) δ 10.35 (s, 1H), 7.05 (s, 1H), 6.95 (d, J=8.8 Hz, 1H), 6.82 (d, J=2.8 Hz, 1H), 6.74 (dd, J=8.8, 2.8 Hz, 1H), 6.65 (d, J=8.4 Hz, 1H), 6.27 (dd, J=8.4, 2.4 Hz, 1H), 6.23 (d, J=2.4 Hz, 1H), 5.95 (td, J=56.8, 4.4 Hz, 1H), 4.44 (s, 2H), 3.65 (d, J=12.0 Hz, 2H), 2.66-2.56 (m, 2H), 2.10 (s, 3H), 2.02-1.85 (m, 1H), 1.79-1.71 (m, 2H), 1.54-1.39 (m, 2H). Mass (m/z): 388.5 [M+H]⁺.

### Compound 655

### 7-[4-[3-(difluoromethyl)azetidin-1-yl]-2-methyl-anilino]-4H-1,4-benzoxazin-3-one

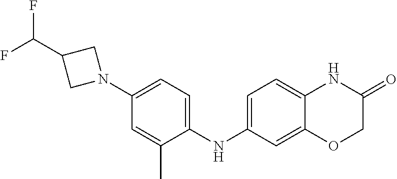 [SMILES: Cc1cc(N2CC(C(F)F)C2)ccc1Nc1ccc2c(c1)OCC(=O)N2]

The titled compound 655 (21 mg, 14%) as an off-white solid was prepared according to the procedure outlined for compound 1. ¹H NMR (400 MHz, DMSO-d₆) δ 10.33 (s, 1H), 7.03 (s, 1H), 6.93 (d, J=8.4 Hz, 1H), 6.63 (d, J=8.4 Hz, 1H), 6.51-6.20 (m, 3H), 6.19 (dd, J=8.4, 2.4 Hz, 1H), 6.15 (d, J=2.4 Hz, 1H), 4.43 (s, 2H), 3.88 (t, J=8.0 Hz, 2H), 3.71 (dd, J=7.6, 5.6 Hz, 2H), 3.24-3.11 (m, 1H), 2.08 (s, 3H). Mass (m/z): 360.5 [M+H]⁺.

### Compound 656

### 5-methyl-7-[2-methyl-4-[4-(trifluoromethyl)-1-piperidyl]anilino]-1,3-dihydro-1,4-benzodiazepin-2-one

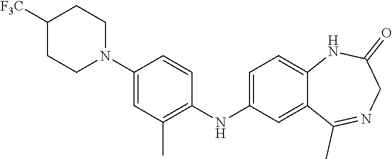 [SMILES: CC1=NCC(=O)Nc2ccc(Nc3ccc(N4CCC(C(F)(F)F)CC4)cc3C)cc21]

The titled compound 656 (15 mg, 9%) as a light yellow solid was prepared according to the procedure outlined for compound 1. ¹H NMR (400 MHz, DMSO-d₆) δ 10.01 (s, 1H), 7.34 (s, 1H), 7.02 (d, J=8.8 Hz, 1H), 6.91 (d, J=8.8 Hz, 1H), 6.88-6.84 (m, 2H), 6.81 (dd, J=8.8, 2.8 Hz, 1H), 6.77 (dd, J=8.8, 2.8 Hz, 1H), 3.83 (s, 2H), 3.70 (d, J=12.4 Hz, 2H), 2.71-2.60 (m, 2H), 2.47-2.40 (m, 1H), 2.27 (s, 3H), 2.14 (s, 3H), 1.93-1.84 (m, 2H), 1.64-1.49 (m, 2H). Mass (m/z): 431.5 [M+H]⁺.

### Compound 657

### 3-hydroxy-8-[2-methyl-4-[3-(trifluoromethoxy)azetidin-1-yl]anilino]-3,5-dihydro-2H-1,5-benzoxazepin-4-one

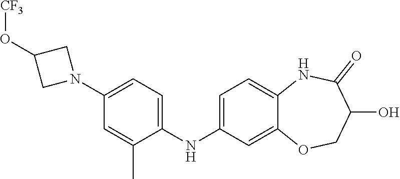 [SMILES: Cc1cc(N2CC(OC(F)(F)F)C2)ccc1Nc1ccc2c(c1)OCC(O)C(=O)N2]

The titled compound 657 (2.3 mg, 5%) as a light yellow solid was prepared according to the procedure outlined for compound 1. ¹H NMR (400 MHz, DMSO-d₆) δ 10.32 (s, 1H), 7.02 (s, 1H), 6.95 (d, J=8.4 Hz, 1H), 6.59 (d, J=8.4 Hz, 1H), 6.39 (d, J=2.8 Hz, 1H), 6.32 (dd, J=8.4, 2.8 Hz, 1H), 6.22-6.13 (m, 2H), 5.30-5.23 (m, 1H), 4.98 (t, J=5.6 Hz, 1H), 4.48 (dd, J=5.2, 3.2 Hz, 1H), 4.19 (dd, J=8.8, 6.4 Hz, 2H), 3.82 (dd, J=8.8, 4.0 Hz, 2H), 3.79-3.65 (m, 2H), 2.09 (s, 3H). Mass (m/z): 424.5 [M+H]⁺.

### Compound 658

### 8-[4-[3-(difluoromethoxy)azetidin-1-yl]-2-methyl-anilino]-3-hydroxy-3,5-dihydro-2H-1,5-benzoxazepin-4-one

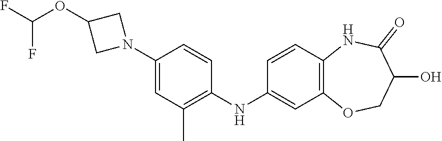 [SMILES: Cc1cc(N2CC(OC(F)F)C2)ccc1Nc1ccc2c(c1)OCC(O)C(=O)N2]

The titled compound 658 (6.8 mg, 10%) as a pink solid was prepared according to the procedure outlined for compound 1. ¹H NMR (400 MHz, DMSO-d₆) δ 10.32 (s, 1H), 7.01 (s, 1H), 6.94 (d, J=8.4 Hz, 1H), 6.78 (t, J=74.8 Hz, 1H), 6.61-6.57 (m, 1H), 6.37 (d, J=2.8 Hz, 1H), 6.30 (dd, J=8.4, 2.8 Hz, 1H), 6.18 (dd, J=8.4, 2.4 Hz, 1H), 6.15 (d, J=2.4 Hz, 1H), 5.09-4.95 (m, 2H), 4.51-4.44 (m, 1H), 4.17-4.09 (m, 2H), 3.80-3.65 (m, 4H), 2.09 (s, 3H). Mass (m/z): 406.5 [M+H]⁺.

### Compound 659

### 7-[4-(1,1-dimethylpropylamino)-2-methyl-anilino]-4H-1,4-benzoxazin-3-one

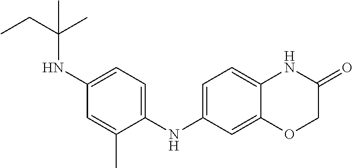 [SMILES: CCC(C)(C)Nc1ccc(Nc2ccc3c(c2)OCC(=O)N3)c(C)c1]

The titled compound 659 (14 mg, 7%) as a light brown solid was prepared according to the procedure outlined for compound 1. ¹H NMR (400 MHz, DMSO-d₆) δ 10.32 (s, 1H), 6.93 (s, 1H), 6.80 (d, J=8.4 Hz, 1H), 6.66-6.57 (m, 2H), 6.53 (dd, J=8.4, 2.8 Hz, 1H), 6.19 (dd, J=8.4, 2.4 Hz, 1H), 6.14 (d, J=2.4 Hz, 1H), 4.66 (s, 1H), 4.43 (s, 2H), 2.03 (s, 3H), 1.60 (q, J=7.2 Hz, 2H), 1.20 (s, 6H), 0.82 (t, J=7.4 Hz, 3H). Mass (m/z): 340.5 [M+H]⁺.

### Example 2. Determination of EC50 Values in HT-1080 Cell Viability Assay

HT-1080 (ATCC, CCL-121) cells (6000 in 100 μl) were seeded in 96-well plates (Corning) and cultured in a 37° C. incubator with a humidified atmosphere of 5% CO₂ for overnight. Cells were then treated with ferroptosis inducer RSL3 (TargetMol) and a 3-fold, 10-point serial dilution series of compounds with 1.1 μM as the highest concentration, for hours. Cell viability was assessed using the Cell Titer-Glo Kit (Promega). The luminescence intensity was measured with a microplate reader (Envision, PerkinElmer), and cell viability was calculated by normalizing the data to untreated controls. The EC50 values of the compounds (e.g., Compounds 1-659) were calculated in GraphPad Prism (GraphPad Software, Inc., San Diego, CA, USA). The curves were fitted using a non-linear regression model with a sigmoidal dose response.

Ferroptosis inhibitory activity of compounds 1-447 is summarized in Table 2. In Table 2, activity is provided as follows: +++=0.1 nM≤EC50<100 nM; ++=100 nM≤EC50<1000 nM; +=1000 nM≤EC50<10000 nM.

TABLE 2
EC50 Values of
Compounds 1 to 447

| Cmpd No. | EC₅₀ (nM) |
|---|---|
| 1 | +++ |
| 2 | +++ |
| 3 | +++ |
| 4 | +++ |
| 5 | +++ |
| 6 | +++ |
| 7 | +++ |
| 8 | +++ |
| 9 | +++ |
| 10 | +++ |
| 11 | +++ |
| 12 | +++ |
| 13 | +++ |
| 14 | +++ |
| 15 | +++ |
| 16 | +++ |
| 17 | +++ |
| 18 | +++ |
| 19 | +++ |
| 20 | +++ |
| 21 | +++ |
| 22 | +++ |
| 23 | +++ |
| 24 | +++ |
| 25 | +++ |
| 26 | +++ |
| 27 | +++ |
| 28 | +++ |
| 29 | +++ |
| 30 | +++ |
| 31 | +++ |
| 32 | +++ |
| 33 | +++ |
| 34 | ++ |
| 35 | +++ |
| 36 | +++ |
| 37 | +++ |
| 38 | +++ |
| 39 | ++ |
| 40 | +++ |
| 41 | +++ |
| 42 | +++ |
| 43 | +++ |
| 44 | +++ |
| 45 | +++ |
| 46 | +++ |
| 47 | +++ |
| 48 | +++ |
| 49 | +++ |
| 50 | +++ |
| 51 | +++ |
| 52 | +++ |
| 53 | +++ |
| 54 | +++ |
| 55 | +++ |
| 56 | +++ |
| 57 | +++ |
| 58 | +++ |
| 59 | +++ |
| 60 | +++ |
| 61 | +++ |
| 62 | +++ |
| 63 | +++ |
| 64 | +++ |
| 65 | +++ |
| 66 | +++ |
| 67 | +++ |
| 68 | +++ |
| 69 | +++ |
| 70 | +++ |
| 71 | +++ |
| 72 | +++ |
| 73 | +++ |
| 74 | +++ |
| 75 | +++ |
| 76 | +++ |
| 77 | +++ |
| 78 | +++ |
| 79 | +++ |
| 80 | +++ |
| 81 | +++ |
| 82 | +++ |
| 83 | +++ |
| 84 | +++ |
| 85 | +++ |
| 86 | +++ |
| 87 | +++ |
| 88 | +++ |
| 89 | +++ |
| 90 | +++ |
| 91 | +++ |
| 92 | +++ |
| 93 | +++ |
| 94 | +++ |
| 95 | +++ |
| 96 | +++ |
| 97 | +++ |
| 98 | +++ |
| 99 | +++ |
| 100 | +++ |
| 101 | ++ |
| 102 | +++ |
| 103 | ++ |
| 104 | +++ |
| 105 | +++ |
| 106 | +++ |
| 107 | +++ |
| 108 | +++ |
| 109 | +++ |
| 110 | +++ |
| 111 | +++ |
| 112 | +++ |
| 113 | +++ |
| 114 | +++ |
| 115 | +++ |
| 116 | +++ |
| 117 | +++ |
| 118 | +++ |
| 119 | +++ |
| 120 | +++ |
| 121 | +++ |
| 122 | +++ |
| 123 | +++ |
| 124 | +++ |
| 125 | +++ |
| 126 | +++ |
| 127 | +++ |
| 128 | +++ |
| 129 | +++ |
| 130 | +++ |
| 131 | +++ |
| 132 | +++ |
| 133 | ++ |
| 134 | +++ |
| 135 | +++ |
| 136 | +++ |
| 137 | +++ |
| 138 | +++ |
| 139 | +++ |
| 140 | +++ |
| 141 | +++ |
| 142 | +++ |
| 143 | +++ |
| 144 | +++ |
| 145 | +++ |
| 146 | +++ |
| 147 | +++ |
| 148 | +++ |
| 149 | +++ |
| 150 | +++ |
| 151 | +++ |
| 152 | ++ |
| 153 | +++ |
| 154 | +++ |
| 155 | +++ |
| 156 | +++ |
| 157 | +++ |
| 158 | +++ |
| 159 | +++ |
| 160 | +++ |
| 161 | +++ |
| 162 | +++ |
| 163 | +++ |
| 164 | +++ |
| 165 | +++ |
| 166 | +++ |
| 167 | +++ |
| 168 | +++ |
| 169 | +++ |
| 170 | +++ |
| 171 | +++ |
| 172 | +++ |
| 173 | +++ |
| 174 | +++ |
| 175 | +++ |
| 176 | +++ |
| 177 | +++ |
| 178 | +++ |
| 179 | +++ |
| 180 | +++ |
| 181 | +++ |
| 182 | +++ |
| 183 | +++ |
| 184 | +++ |
| 185 | +++ |
| 186 | +++ |
| 187 | +++ |
| 188 | ++ |
| 189 | +++ |
| 190 | +++ |
| 191 | +++ |
| 192 | +++ |
| 193 | +++ |
| 194 | +++ |
| 195 | +++ |
| 196 | +++ |
| 197 | +++ |
| 198 | +++ |
| 199 | +++ |
| 200 | +++ |
| 201 | +++ |
| 202 | +++ |
| 203 | +++ |
| 204 | +++ |
| 205 | +++ |
| 206 | +++ |
| 207 | +++ |
| 208 | +++ |
| 209 | +++ |
| 210 | +++ |
| 211 | +++ |
| 212 | +++ |
| 213 | +++ |
| 214 | +++ |
| 215 | +++ |
| 216 | +++ |
| 217 | +++ |
| 218 | +++ |
| 219 | +++ |
| 220 | +++ |
| 221 | +++ |
| 222 | +++ |
| 223 | +++ |
| 224 | +++ |
| 225 | +++ |
| 226 | +++ |
| 227 | +++ |
| 228 | +++ |
| 229 | +++ |
| 230 | +++ |
| 231 | +++ |
| 232 | +++ |
| 233 | ++ |
| 234 | +++ |
| 235 | +++ |
| 236 | +++ |
| 237 | +++ |
| 238 | + |
| 239 | +++ |
| 240 | +++ |
| 241 | +++ |
| 242 | +++ |
| 243 | +++ |
| 244 | +++ |
| 245 | +++ |
| 246 | +++ |
| 247 | +++ |
| 248 | +++ |
| 249 | +++ |
| 250 | +++ |
| 251 | +++ |
| 252 | +++ |
| 253 | +++ |
| 254 | +++ |
| 255 | +++ |
| 256 | +++ |
| 257 | +++ |
| 258 | +++ |
| 259 | +++ |
| 260 | +++ |
| 261 | +++ |
| 262 | +++ |
| 263 | +++ |
| 264 | +++ |
| 265 | +++ |
| 266 | +++ |
| 267 | +++ |
| 268 | +++ |
| 269 | +++ |
| 270 | +++ |
| 271 | +++ |
| 272 | +++ |
| 273 | +++ |
| 274 | +++ |
| 275 | +++ |
| 276 | +++ |
| 277 | +++ |
| 278 | +++ |
| 279 | +++ |
| 280 | +++ |
| 281 | +++ |
| 282 | +++ |
| 283 | +++ |
| 284 | +++ |
| 285 | +++ |
| 286 | +++ |
| 287 | +++ |
| 288 | +++ |
| 289 | +++ |
| 290 | +++ |
| 291 | +++ |
| 292 | + |
| 293 | +++ |
| 294 | +++ |
| 295 | + |
| 296 | +++ |
| 297 | +++ |
| 298 | +++ |
| 299 | +++ |
| 300 | +++ |
| 301 | +++ |
| 302 | +++ |
| 303 | +++ |
| 304 | +++ |
| 305 | ++ |
| 306 | ++ |
| 307 | ++ |
| 308 | +++ |
| 309 | +++ |
| 310 | +++ |
| 311 | +++ |
| 312 | +++ |
| 313 | +++ |
| 314 | +++ |
| 315 | +++ |
| 316 | +++ |
| 317 | +++ |
| 318 | +++ |
| 319 | +++ |
| 320 | +++ |
| 321 | +++ |
| 322 | +++ |
| 323 | +++ |
| 324 | +++ |
| 325 | +++ |
| 326 | +++ |
| 327 | +++ |
| 328 | +++ |
| 329 | +++ |
| 330 | +++ |
| 331 | ++ |
| 332 | +++ |
| 333 | +++ |
| 334 | +++ |
| 335 | +++ |
| 336 | +++ |
| 337 | +++ |
| 338 | +++ |
| 339 | +++ |
| 340 | +++ |
| 341 | +++ |
| 342 | ++ |
| 343 | +++ |
| 344 | +++ |
| 345 | +++ |
| 346 | ++ |
| 347 | +++ |
| 348 | +++ |
| 349 | +++ |
| 350 | +++ |
| 351 | +++ |
| 352 | +++ |
| 353 | +++ |
| 354 | +++ |
| 355 | +++ |
| 356 | +++ |
| 357 | +++ |
| 358 | +++ |
| 359 | +++ |
| 360 | +++ |
| 361 | ++ |
| 362 | +++ |
| 363 | +++ |
| 364 | +++ |
| 365 | +++ |
| 366 | +++ |
| 367 | +++ |
| 368 | +++ |
| 369 | ++ |
| 370 | +++ |
| 371 | +++ |
| 372 | +++ |
| 373 | +++ |
| 374 | +++ |
| 375 | +++ |
| 376 | +++ |
| 377 | +++ |
| 378 | +++ |
| 379 | +++ |
| 380 | +++ |
| 381 | +++ |
| 382 | +++ |
| 383 | +++ |
| 384 | +++ |
| 385 | +++ |
| 386 | +++ |
| 387 | +++ |
| 388 | +++ |
| 389 | +++ |
| 390 | +++ |
| 391 | +++ |
| 392 | +++ |
| 393 | +++ |
| 394 | +++ |
| 395 | +++ |
| 396 | +++ |
| 397 | +++ |
| 398 | +++ |
| 399 | +++ |
| 400 | +++ |
| 401 | +++ |
| 402 | +++ |
| 403 | +++ |
| 404 | +++ |
| 405 | +++ |
| 406 | +++ |
| 407 | +++ |
| 408 | +++ |
| 409 | +++ |
| 410 | +++ |
| 411 | +++ |
| 412 | +++ |
| 413 | +++ |
| 414 | +++ |
| 415 | +++ |
| 416 | +++ |
| 417 | +++ |
| 418 | +++ |
| 419 | +++ |
| 420 | +++ |
| 421 | +++ |
| 422 | +++ |
| 423 | +++ |
| 424 | +++ |
| 425 | +++ |
| 426 | +++ |
| 427 | +++ |
| 428 | +++ |
| 429 | +++ |
| 430 | +++ |
| 431 | +++ |
| 432 | +++ |
| 433 | +++ |
| 434 | +++ |
| 435 | +++ |
| 436 | +++ |
| 437 | +++ |
| 438 | +++ |
| 439 | +++ |
| 440 | +++ |
| 441 | +++ |
| 442 | +++ |
| 443 | +++ |
| 444 | +++ |
| 445 | +++ |
| 446 | +++ |
| 447 | +++ |

Ferroptosis inhibitory activity of compounds 448-659 is summarized in Table 3. In Table 3, activity is provided as follows: +++=0.1 nM≤EC50<100 nM; ++=100 nM≤EC50<1000 nM; +=1000 nM≤EC50<10000 nM.

TABLE 3
EC50 Values of
Compounds 448 to 659

| Cmpd No. | EC₅₀ (nM) |
|---|---|
| 448 | +++ |
| 449 | +++ |
| 450 | +++ |
| 451 | +++ |
| 452 | +++ |
| 453 | +++ |
| 454 | +++ |
| 455 | +++ |
| 456 | +++ |
| 457 | +++ |
| 458 | +++ |
| 459 | +++ |
| 460 | +++ |
| 461 | +++ |
| 462 | +++ |
| 463 | +++ |
| 464 | +++ |
| 465 | +++ |
| 466 | +++ |
| 467 | +++ |
| 468 | +++ |
| 469 | +++ |
| 470 | +++ |
| 471 | +++ |
| 472 | +++ |
| 473 | +++ |
| 474 | +++ |
| 475 | +++ |
| 476 | +++ |
| 477 | +++ |
| 478 | +++ |
| 479 | +++ |
| 480 | +++ |
| 481 | +++ |
| 482 | +++ |
| 483 | +++ |
| 484 | +++ |
| 485 | +++ |
| 486 | +++ |
| 487 | +++ |
| 488 | +++ |
| 489 | +++ |
| 490 | +++ |
| 491 | +++ |
| 492 | +++ |
| 493 | +++ |
| 494 | +++ |
| 495 | +++ |
| 496 | +++ |
| 497 | +++ |
| 498 | +++ |
| 499 | +++ |
| 500 | +++ |
| 501 | +++ |
| 502 | +++ |
| 503 | +++ |
| 504 | +++ |
| 505 | +++ |
| 506 | +++ |
| 507 | +++ |
| 508 | +++ |
| 509 | +++ |
| 510 | +++ |
| 511 | +++ |
| 512 | +++ |
| 513 | +++ |
| 514 | +++ |
| 515 | +++ |
| 516 | +++ |
| 517 | +++ |
| 518 | +++ |
| 519 | +++ |
| 520 | +++ |
| 521 | +++ |
| 522 | +++ |
| 523 | +++ |
| 524 | +++ |
| 525 | +++ |
| 526 | +++ |
| 527 | +++ |
| 528 | +++ |
| 529 | +++ |
| 530 | +++ |
| 531 | +++ |
| 532 | ++ |
| 533 | +++ |
| 534 | +++ |
| 535 | +++ |
| 536 | +++ |
| 537 | +++ |
| 538 | +++ |
| 539 | +++ |
| 540 | ++ |
| 541 | ++ |
| 542 | +++ |
| 543 | +++ |
| 544 | +++ |
| 545 | +++ |
| 546 | +++ |
| 547 | +++ |
| 548 | +++ |
| 549 | +++ |
| 550 | +++ |
| 551 | +++ |
| 552 | +++ |
| 553 | +++ |
| 554 | +++ |
| 555 | +++ |
| 556 | +++ |
| 557 | +++ |
| 558 | +++ |
| 559 | +++ |
| 560 | +++ |
| 561 | +++ |
| 562 | +++ |
| 563 | +++ |
| 564 | +++ |
| 565 | +++ |
| 566 | +++ |
| 567 | +++ |
| 568 | +++ |
| 569 | +++ |
| 570 | +++ |
| 571 | +++ |
| 572 | +++ |
| 573 | +++ |
| 574 | +++ |
| 575 | +++ |
| 576 | +++ |
| 577 | +++ |
| 578 | +++ |
| 579 | +++ |
| 580 | +++ |
| 581 | +++ |
| 582 | +++ |
| 583 | +++ |
| 584 | +++ |
| 585 | +++ |
| 586 | +++ |
| 587 | +++ |
| 588 | +++ |
| 589 | +++ |
| 590 | +++ |
| 591 | +++ |
| 592 | +++ |
| 593 | +++ |
| 594 | +++ |
| 595 | +++ |
| 596 | +++ |
| 597 | +++ |
| 598 | +++ |
| 599 | +++ |
| 600 | +++ |
| 601 | +++ |
| 602 | +++ |
| 603 | +++ |
| 604 | +++ |
| 605 | +++ |
| 606 | ++ |
| 607 | +++ |
| 608 | +++ |
| 609 | +++ |
| 610 | +++ |
| 611 | +++ |
| 612 | +++ |
| 613 | +++ |
| 614 | +++ |
| 615 | + |
| 616 | +++ |
| 617 | +++ |
| 618 | +++ |
| 619 | +++ |
| 620 | +++ |
| 621 | +++ |
| 622 | +++ |
| 623 | +++ |
| 624 | +++ |
| 625 | +++ |
| 626 | +++ |
| 627 | +++ |
| 628 | +++ |
| 629 | +++ |
| 630 | +++ |
| 631 | +++ |
| 632 | +++ |
| 633 | +++ |
| 634 | +++ |
| 635 | +++ |
| 636 | +++ |
| 637 | +++ |
| 638 | +++ |
| 639 | +++ |
| 640 | +++ |
| 641 | +++ |
| 642 | +++ |
| 643 | +++ |
| 644 | +++ |
| 645 | +++ |
| 646 | +++ |
| 647 | +++ |
| 648 | +++ |
| 649 | +++ |
| 650 | +++ |
| 651 | +++ |
| 652 | +++ |
| 653 | +++ |
| 654 | +++ |
| 655 | +++ |
| 656 | +++ |
| 657 | +++ |
| 658 | +++ |
| 659 | +++ |

All publications, including but not limited to disclosures and disclosure applications, cited in this specification are herein incorporated by reference as though fully set forth. If certain content of a publication cited herein contradicts or is inconsistent with the present disclosure, the present disclosure controls.

One skilled in the art will readily recognize from the disclosure and claims that various changes, modifications, and variations can be made therein without departing from the spirit and scope of the disclosure as defined in the following claims.

1. A compound of the following structural Formula I: 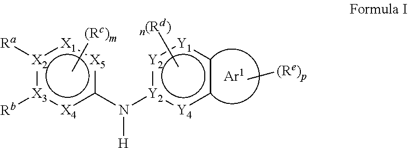
a tautomer thereof, a solvate or stereoisomer of the compound or the tautomer, or a pharmaceutically acceptable salt of the foregoing, wherein:
Ar¹ is aryl, cycloalkyl, heteroaryl, or 5- to 7-membered heterocyclyl;
X₁ is C, N, or S;
X₂, X₃, X₅, Y₁, Y₂, Y₃, and Y₄ are each independently C or N;
X₄ is C or N, but when X₁ is S, X₄ is nothing;
R^{a} is selected from H, halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl;
R^{b} is selected from H, halogen, CN, optionally substituted heteroatom, optionally substituted alkyl, optionally substituted cycloalkyl, and optionally substituted heterocyclyl;
R^{a} and R^{b} may join together to form an optionally substituted 3- to 10-membered carbocyclic, heterocyclic, aromatic, or heteroaromatic ring;
R^{c}, for each occurrence, is independently selected from halogen, CN, optionally substituted heteroatom, and optionally substituted alkyl;
R^{d}, for each occurrence, is independently selected from halogen, CN, optionally substituted heteroatom, and optionally substituted alkyl;
R^{e}, for each occurrence, is independently selected from halogen, CN, optionally substituted heteroatom, optionally substituted alkyl, optionally substituted alkenyl, optionally substituted alkynyl, optionally substituted acyl, optionally substituted cycloalkyl, optionally substituted heterocyclyl, optionally substituted aryl, and optionally substituted heteroaryl; and
m, n, and p are each an integer independently selected from 0, 1, 2, and 3;
provided that:
(1) 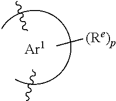
is not 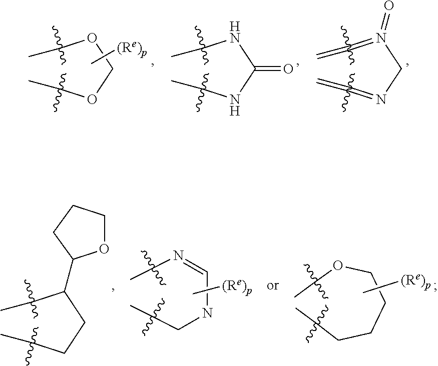
(2) when 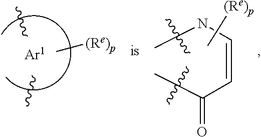
R^{e} is not —C(═O)OH or —C(═O)O(C₁-C₃ alkyl);
(3) when 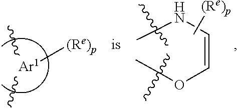
R^{a} is not OH; and
(4) when Ar¹ is a 5- to 6-membered cycloalkyl, 5- to 6-membered heteroaryl, or 5- to 7-membered heterocyclyl, (i) R^{a} is selected from optionally substituted cycloalkyl, optionally substituted heterocyclyl, optionally substituted aryl, and optionally substituted heteroaryl, (ii) R^{a} is selected from H, halogen, CN, optionally substituted heteroatom, optionally substituted alkyl, optionally substituted alkenyl, optionally substituted alkynyl, provided that p is an integer selected from 1, 2, and 3 and that at least one of the R^{e} is ═O, or (iii) R^{a} and R^{b} join together to form an optionally substituted 3- to 10-membered carbocyclic, heterocyclic, aromatic, or heteroaromatic ring, provided that p is an integer selected from 1, 2, and 3 and that at least one of the R^{e} is ═O.

2. The compound, tautomer, solvate, stereoisomer, or pharmaceutically acceptable salt of claim 1, wherein Ar¹ is 5- to 6-membered heteroaryl or 5- to 7-membered heterocyclyl.

3. The compound of claim 1, wherein the compound has the following structural Formula IIa: 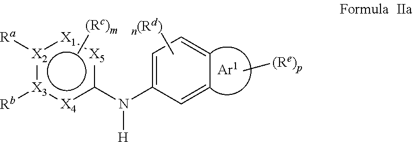
a tautomer thereof, a solvate or stereoisomer of the compound or the tautomer, or a pharmaceutically acceptable salt of the foregoing.

4. The compound of claim 1, wherein the compound has the following structural Formula IIb: 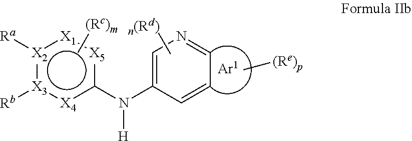
a tautomer thereof, a solvate or stereoisomer of the compound or the tautomer, or a pharmaceutically acceptable salt of the foregoing.

5. The compound of claim 1, wherein the compound has the following structural Formula IIIa: 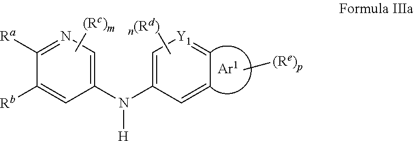
a tautomer thereof, a solvate or stereoisomer of the compound or the tautomer, or a pharmaceutically acceptable salt of the foregoing.

6. The compound of claim 1, wherein the compound has the following structural Formula IIIb: 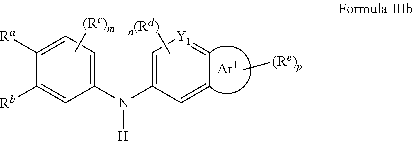
a tautomer thereof, a solvate or stereoisomer of the compound or the tautomer, or a pharmaceutically acceptable salt of the foregoing.

7. The compound of claim 1, wherein the compound has the following structural Formula IIIc: 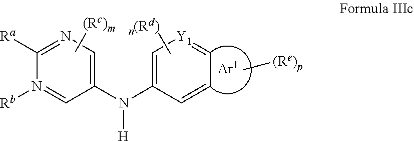
a tautomer thereof, a solvate or stereoisomer of the compound or the tautomer, or a pharmaceutically acceptable salt of the foregoing.

8. The compound of claim 1, wherein the compound has the following structural Formula IIId: 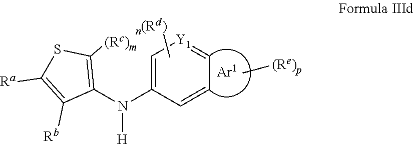
a tautomer thereof, a solvate or stereoisomer of the compound or the tautomer, or a pharmaceutically acceptable salt of the foregoing.

9. The compound of claim 1, wherein the compound has one of the following structural Formula IVa: 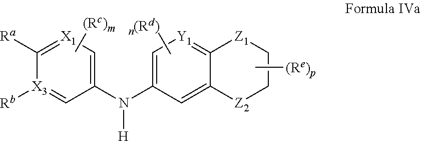
a tautomer thereof, a solvate or stereoisomer of the compound or the tautomer, or a pharmaceutically acceptable salt of the foregoing, wherein X₁ and X₃ are each independently C or N; and Z₁ and Z₂ are each independently N or O.

10. The compound of claim 1, wherein the compound has one of the following structural Formula IVb: 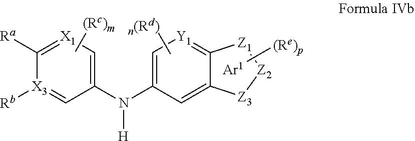
a tautomer thereof, a solvate or stereoisomer of the compound or the tautomer, or a pharmaceutically acceptable salt of the foregoing, wherein X₁ and X₃ are each independently C or N; Ar¹ is aromatic or non-aromatic; and Z₁, Z₂, and Z₃ are each independently N or C.

11. The compound of claim 1, wherein the compound has one of the following structural Formula IVc: 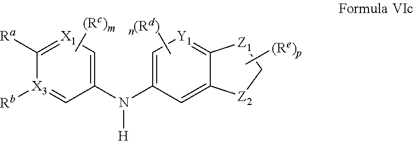
a tautomer thereof, a solvate or stereoisomer of the compound or the tautomer, or a pharmaceutically acceptable salt of the foregoing, wherein X₁ and X₃ are each independently C or N; and Z₁ and Z₂ are each independently O or N.

12. The compound of claim 1, wherein the compound has one of the following structural Formula IVd: 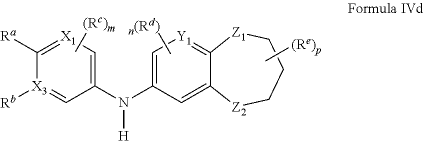
a tautomer thereof, a solvate or stereoisomer of the compound or the tautomer, or a pharmaceutically acceptable salt of the foregoing, wherein X₁ and X₃ are each independently C or N; and Z₁ and Z₂ are each independently O or N.

13. The compound of claim 1, wherein the compound has the following structural Formula Va: 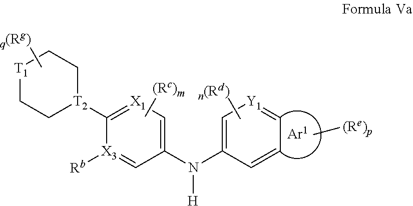
a tautomer thereof, a solvate or stereoisomer of the compound or the tautomer, or a pharmaceutically acceptable salt of the foregoing, wherein X₁ and X₃ are each independently N or C; T₁ is C or O; T₂ is C or N; R^{g}, for each occurrence, is selected from CF₃, —CH₃, —OCH₃, and —CH₂CH₃; q is 0, 1, or 2; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; wherein the C₁ to C₃ alkoxy and C₁ to C₃ alkyl of R^{b} and R^{c}, for each occurrence, are each optionally substituted with 1 to 3 groups of halogen.

14. The compound of claim 1, wherein the compound has the following structural Formula Vb: 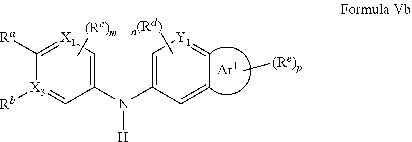
a tautomer thereof, a solvate or stereoisomer of the compound or the tautomer, or a pharmaceutically acceptable salt of the foregoing, wherein X₁ and X₃ are each independently N or C; R^{a} is selected from C₁ to C₃ alkoxy and C₁ to C₃ alkyl; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; wherein the C₁ to C₃ alkoxy and C₁ to C₃ alkyl of R^{a}, R^{b}, and R^{c}, for each occurrence, are each optionally substituted with 1 to 3 groups of halogen.

15. The compound of claim 1, wherein the compound has the following structural Formula VIa: 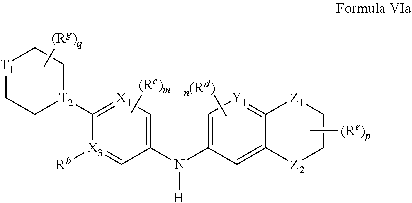
a tautomer thereof, a solvate or stereoisomer of the compound or the tautomer, or a pharmaceutically acceptable salt of the foregoing, wherein X₁ and X₃ are each independently N or C; T₁ is C or O; T₂ is C or N; R^{g}, for each occurrence, is independently selected from CF₃, —CH₃, —OCH₃, and —CH₂CH₃; q is 0, 1, or 2; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; the C₁ to C₃ alkoxy and C₁ to C₃ alkyl of R^{b} and R^{c}, for each occurrence, are each optionally substituted with 1 to 3 groups of halogen; Z₁ and Z₂ are each independently N or O.

16. The compound of claim 1, wherein the compound has the following structural Formula VIb: 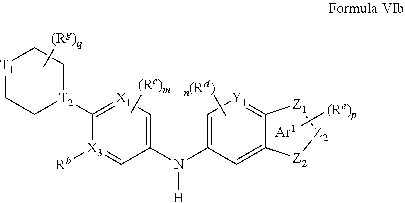
a tautomer thereof, a solvate or stereoisomer of the compound or the tautomer, or a pharmaceutically acceptable salt of the foregoing, wherein X₁ and X₃ are each independently N or C; T₁ is C or O; T₂ is C or N; R^{g}, for each occurrence, is independently selected from CF₃, —CH₃, —OCH₃, and —CH₂CH₃; q is 0, 1, or 2; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; the C₁ to C₃ alkoxy and C₁ to C₃ alkyl of R^{b} and R^{c}, for each occurrence, are each optionally substituted with 1 to 3 groups of halogen; Ar is aromatic or non-aromatic; and Z₁, Z₂, and Z₃ are each independently N or C.

17. The compound of claim 1, wherein the compound has the following structural Formula VIc: 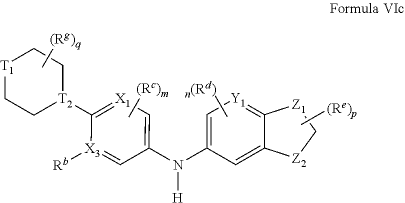
a tautomer thereof, a solvate or stereoisomer of the compound or the tautomer, or a pharmaceutically acceptable salt of the foregoing, wherein X₁ and X₃ are each independently N or C; T₁ is C or O; T₂ is C or N; R^{g}, for each occurrence, is independently selected from CF₃, —CH₃, —OCH₃, and —CH₂CH₃; q is 0, 1, or 2; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; the C₁ to C₃ alkoxy and C₁ to C₃ alkyl of R^{b} and R^{c}, for each occurrence, are each optionally substituted with 1 to 3 groups of halogen; and Z₁ and Z₂ are each independently O or N.

18. The compound of claim 1, wherein the compound has the following structural Formula VId: 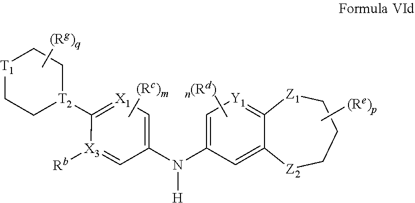
a tautomer thereof, a solvate or stereoisomer of the compound or the tautomer, or a pharmaceutically acceptable salt of the foregoing, wherein X₁ and X₃ are each independently N or C; T₁ is C or O; T₂ is C or N; R^{g}, for each occurrence, is independently selected from CF₃, —CH₃, —OCH₃, and —CH₂CH₃; q is 0, 1, or 2; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; the C₁ to C₃ alkoxy and C₁ to C₃ alkyl of R^{b} and R^{c}, for each occurrence, are each optionally substituted with 1 to 3 groups of halogen; and Z₁ and Z₂ are each independently O or N.

19. The compound of claim 1, wherein the compound has one of the following structural Formula VIIa: 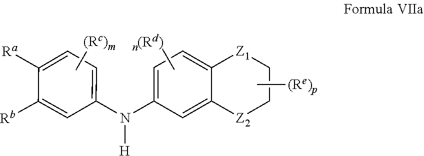
a tautomer thereof, a solvate or stereoisomer of the compound or the tautomer, or a pharmaceutically acceptable salt of the foregoing, wherein Z₁ and Z₂ are each independently N or O; R^{a} is selected from C₁ to C₃ alkoxy and C₁ to C₃ alkyl; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; wherein the C₁ to C₃ alkoxy and C₁ to C₃ alkyl of R^{a}, R^{b}, and R^{c}, for each occurrence, are each optionally substituted with 1 to 3 groups of halogen.

20. The compound of claim 1, wherein the compound has one of the following structural Formula VIIb: 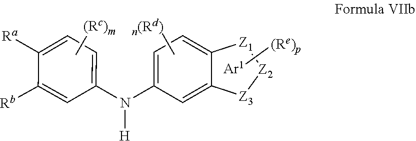
a tautomer thereof, a solvate or stereoisomer of the compound or the tautomer, or a pharmaceutically acceptable salt of the foregoing, wherein Ar¹ is aromatic or non-aromatic; Z₁, Z₂, and Z₃ are each independently N or C; R^{a} is selected from C₁ to C₃ alkoxy and C₁ to C₃ alkyl; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; wherein the C₁ to C₃ alkoxy and C₁ to C₃ alkyl of R^{a}, R^{b}, and R^{c}, for each occurrence, are each optionally substituted with 1 to 3 groups of halogen.

21. The compound of claim 1, wherein the compound has one of the following structural Formula VIIc: 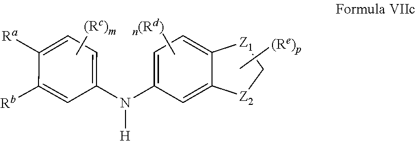
a tautomer thereof, a solvate or stereoisomer of the compound or the tautomer, or a pharmaceutically acceptable salt of the foregoing, wherein Z₁ and Z₂ are each independently O or N; R^{a} is selected from C₁ to C₃ alkoxy and C₁ to C₃ alkyl; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; wherein the C₁ to C₃ alkoxy and C₁ to C₃ alkyl of R^{a}, R^{b}, and R^{c}, for each occurrence, are each optionally substituted with 1 to 3 groups of halogen.

22. The compound of claim 1, wherein the compound has one of the following structural Formula VIId: 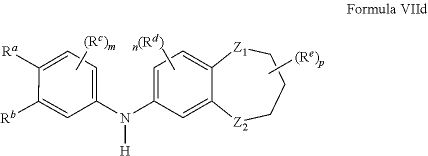
a tautomer thereof, a solvate or stereoisomer of the compound or the tautomer, or a pharmaceutically acceptable salt of the foregoing, wherein Z₁ and Z₂ are each independently O or N; R^{a} is selected from C₁ to C₃ alkoxy and C₁ to C₃ alkyl; R^{b} is selected from H, halogen, C₁ to C₃ alkoxy, and C₁ to C₃ alkyl; and R^{c} is selected from halogen, C₁ to C₃ alkoxy, and C₁ to C₃ alkyl; wherein the C₁ to C₃ alkoxy and C₁ to C₃ alkyl of R^{a}, R^{b}, and R^{c}, for each occurrence, are each optionally substituted with 1 to 3 groups of halogen.

23. The compound of claim 1, wherein the compound has one of the following structural Formulae VIIIa-VIIII: 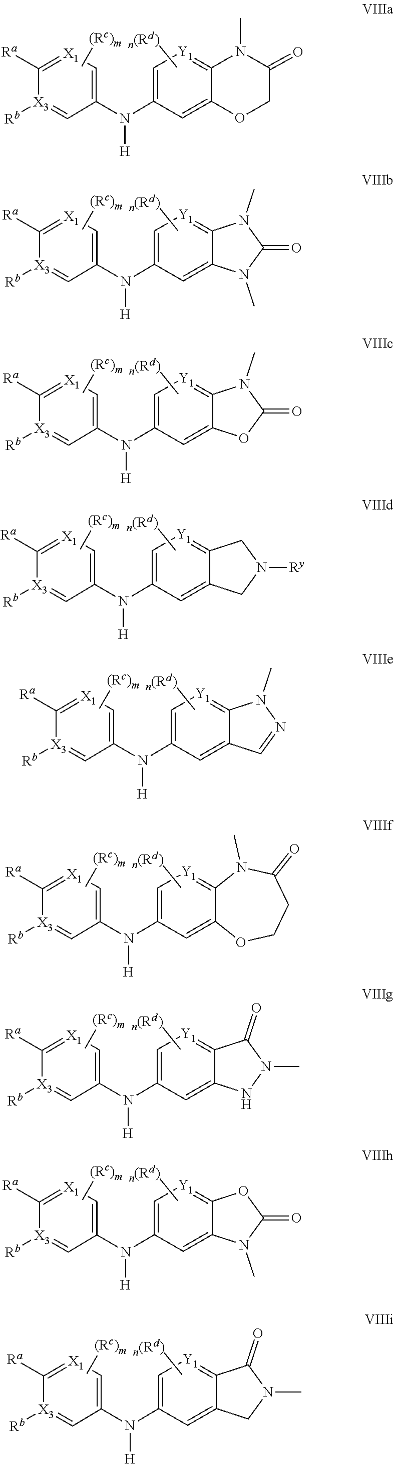 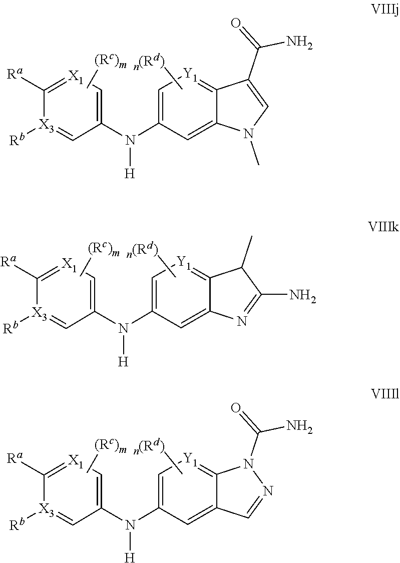
a tautomer thereof, a solvate or stereoisomer of the compound or the tautomer, or a pharmaceutically acceptable salt of the foregoing, wherein:
R^{a} is selected from 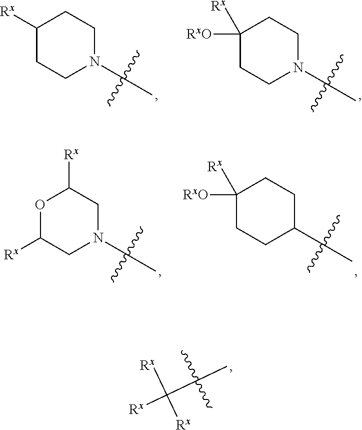
—OR^{x}, and —CH₂R^{x}, wherein R^{x}, for each occurence, is independently selected from CF₃, —CH₃, and —CH₂CH₃;
R^{b} is selected from H, halogen, C₁ to C₂ alkyl, and C₁ to C₂ alkoxy;
R^{c} is selected from halogen, C₁ to C₂ alkyl, and C₁ to C₂ alkoxy;
R^{d} is selected from halogen, C₁ to C₂ alkyl, and C₁ to C₂ alkoxy;
R^{y} is selected from H and C₁ to C₂ alkyl;
m is 0, 1, or 2; and
n is 0 or 1.

24. The compound, tautomer, solvate, stereoisomer, or pharmaceutically acceptable salt of claim 1, R^{a} is selected from hydrogen, halogen, cyano, C₁-C₁₀ alkyl, C₂-C₁₀ alkenyl, C₂-C₁₀ alkynyl, C₃-C₁₀ cycloalkyl, 3- to 10-membered heterocyclyl, phenyl, 5- to 7-membered heteroaryl, —C(═O)R^{s}, —C(═O)OR^{s}, —NR^{p}R^{q}, —OR^{s}, wherein
the C₃-C₁₀ cycloalkyl, 3- to 10-membered heterocyclyl, phenyl, and 5- to 7-membered heteroaryl of R^{a} are each optionally substituted with 1-3 groups selected from halogen, OR^{s}, and C₁-C₁₀ alkyl optionally substituted with 1 to 3 groups of halogen;
the C₁1-C₁₀ alkyl, C₂-C₁₀ alkenyl, and C₂-C₁₀ alkynyl of R^{a} are each optionally substituted with 1 to 3 groups selected from halogen, 3- to 10-membered heterocyclyl, and —OR^{s};
R^{p} and R^{q}, for each occurrence, are independently selected from hydrogen, C₃-C₁₀ cycloalkyl, and C₁-C₁₀ alkyl, wherein the C₃-C₁₀ cycloalkyl and C₁-C₁₀ alkyl of R^{p} and R^{q} are each optionally substituted with 1 to 3 groups selected from halogen, —OR^{s} and —C(═O)OR^{s};
R^{s}, for each occurrence, is selected from H, C₃-C₁₀ cycloalkyl, 3- to 10-membered heterocyclyl, 5- to 6-membered aryl, and C₁-C₁₀ alkyl optionally substituted with 1 to 3 groups selected from halogen, C₃-C₁₀ cycloalkyl, 3- to 10-membered heterocyclyl, and C₁-C₆ alkoxy, wherein the C₃-C₁₀ cycloalkyl and 3- to 10-membered heterocyclyl of R^{s} and the C₃-C₁₀ cycloalkyl and 3- to 10-membered heterocyclyl of the C₁-C₁₀ alkyl of R^{s} are each optionally substituted with 1 to 3 groups of C₁-C₆ alkyl optionally substituted with halogen, and the C₁-C₆ alkoxy of the C₁-C₁₀ alkyl of R^{s} is optionally substituted with 1 to 3 groups of halogen.

25. The compound, tautomer, solvate, stereoisomer, or pharmaceutically acceptable salt of claim 1, wherein R^{a} is selected from H, F, methyl, 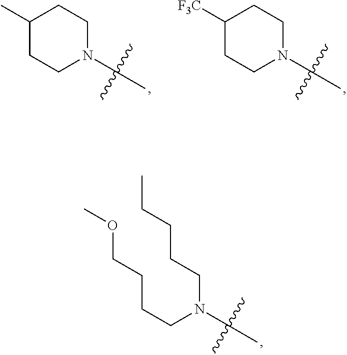
—O(CH₂)₃CH₃, —(CH₂)₄CH₃, 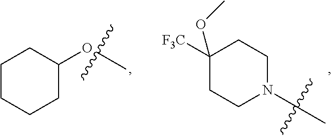
—O(CH₂)₂CH₂F, 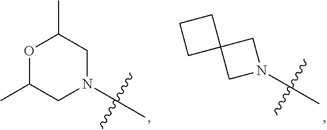
—N(CH₂CH₃)₂, 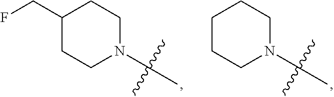 [SMILES: *N1CCC(CF)CC1 ; *N1CCCCC1]
—N(CH₂CH₃)(CH₂)₄CH₃, —N(CH₂CH₃)(CH₂)₃OCH₃, —O(CH₂)₄CH₃, 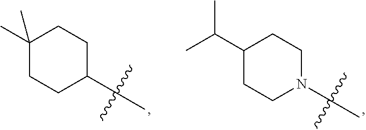
—C(CH₃)₂CH₂CH₃, —C(CH₃)₃, 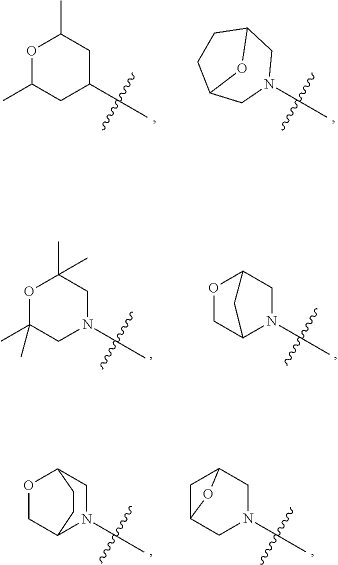
—N(CH₃)CH₂CF₃, 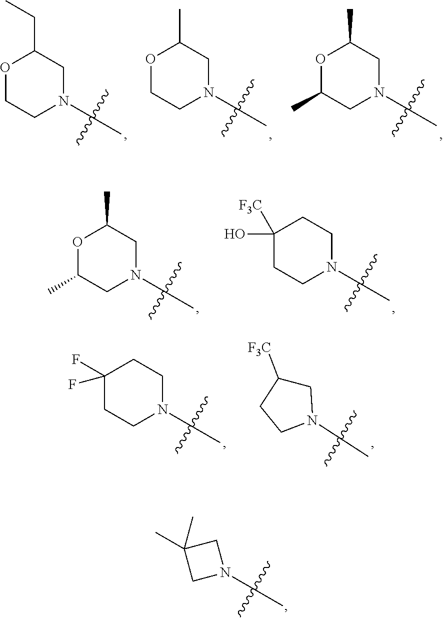
—N(CH₃)₂, 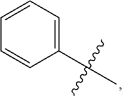 [SMILES: *c1ccccc1]
—O(CH₂)₂OCH₃, 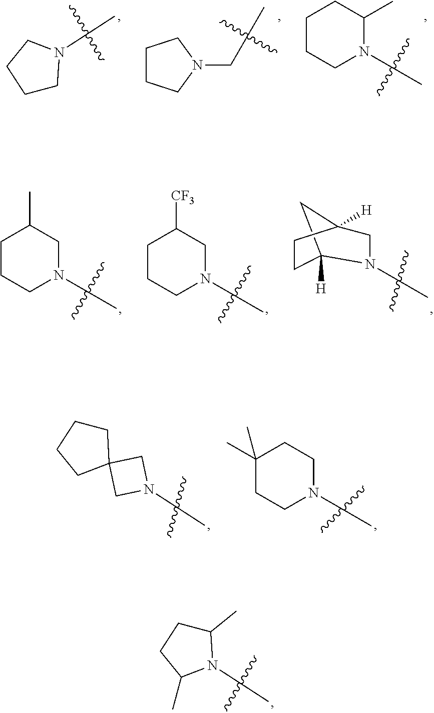
—C(═O)CF, —N(CH₂CH₃)(CH₂)₄CH₃, —N(CH₂CH₃)(CH₂)₃OH₃, 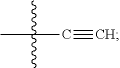 [SMILES: *C#C]
—O(CH₂)₄CH₃, —OCH₂CH₃, 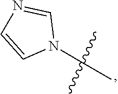 [SMILES: *n1ccnc1]
—C(═O)OCH₂CH₃, —OCH₃, 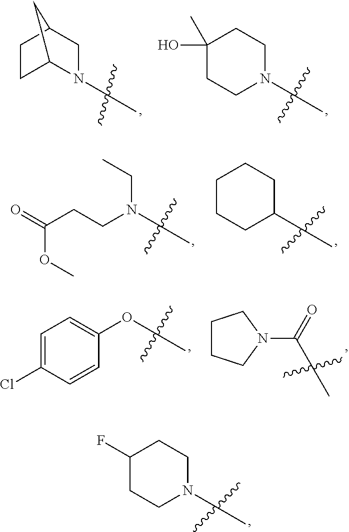
and —N(CH₃)(CH₂CH₂OCH₂OCH₂CH₃).

26. The compound, tautomer, solvate, stereoisomer, or pharmaceutically acceptable salt of claim 1, wherein R^{a} is selected from 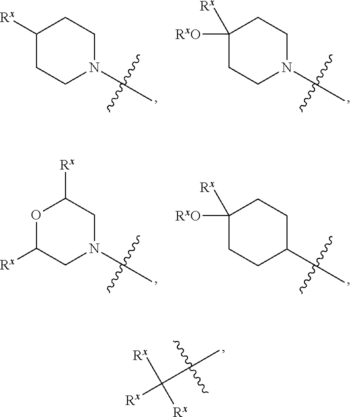
—OR^{x}, and —CH₂R^{x}, wherein R^{x}, for each occurrence, is independently selected from C₁ to C₂ alkyl optionally substituted with 1 to 3 groups of halogen.

27. The compound, tautomer, solvate, stereoisomer, or pharmaceutically acceptable salt of claim 1, wherein R^{a} is selected from 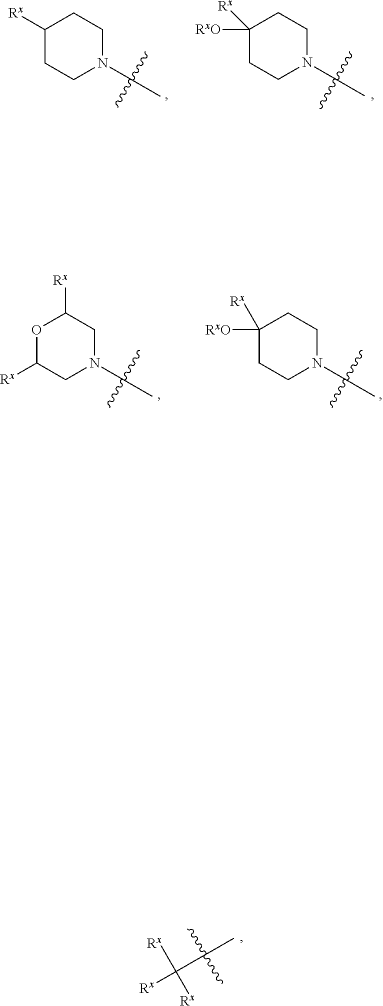
—OR^{x}, and —CH₂R^{x}, wherein R^{x}, for each occurrence, is independently selected from CF₃, —CH₃, and —CH₂CH₃.

28. The compound, tautomer, solvate, stereoisomer, or pharmaceutically acceptable salt of claim 1, wherein R^{b} is selected from is H, halogen, C₁ to C₆ alkyl, —NR^{p}R^{q}, CN, C₁-C₆ alkoxy, and 5- to 6-membered heterocyclyl optionally substituted with C₁-C₃ alkyl optionally substituted with 1 to 3 groups selected from halogen, wherein R^{p} and R^{q} are each selected from C₁ to C₆ alkyl, and wherein the C₁-C₆ alkyl and C₁-C₆ alkoxy of R^{b} and the C₁-C₆ alkyl of R^{p} and R^{q} are each optionally substituted with 1 to 3 groups selected from halogen.

29. The compound, tautomer, solvate, stereoisomer, or pharmaceutically acceptable salt of claim 1, wherein R^{b} is H, methyl, —NHCH₃, —N(CH₃)₂, —O(CH₂)₂CH₃, F, —OCH₃, —CF₃, Cl, —N(CH₃)₂, and

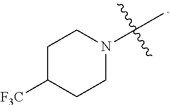 [SMILES: *N1CCC(C(F)(F)F)CC1]

30. The compound, tautomer, solvate, stereoisomer, or pharmaceutically acceptable salt of claim 1, wherein R^{b} is selected from halogen, C₁-C₂ alkyl, and C₁-C₂ alkoxy.

31. The compound, tautomer, solvate, stereoisomer, or pharmaceutically acceptable salt of claim 1, wherein R^{b} is selected from F, methyl, and —OCH₃.

32. The compound, tautomer, solvate, stereoisomer, or pharmaceutically acceptable salt of claim 1, wherein R^{c}, for each occurrence, is independently selected from is halogen, C₁-C₆ alkyl, —NR^{p}R^{q}, CN, and C₁-C₆ alkoxy, wherein R^{p} and R^{q} are each selected from C₁ to C₆ alkyl, and wherein the C₁-C₆ alkyl and C₁-C₆ alkoxy of R^{c} and the C₁-C₆ alkyl of R^{p} and R^{q} are each optionally substituted with 1 to 3 groups selected from halogen.

33. The compound, tautomer, solvate, stereoisomer, or pharmaceutically acceptable salt of claim 1, wherein R^{c} is selected from methyl, F, —OCH₃, Cl, —N(CH₃)₂, and CN.

34. The compound, tautomer, solvate, stereoisomer, or pharmaceutically acceptable salt of claim 1, wherein R^{c} is selected from halogen, C₁-C₂ alkyl, and C₁-C₂ alkoxy.

35. The compound, tautomer, solvate, stereoisomer, or pharmaceutically acceptable salt of claim 1, wherein R^{c} is selected from F, methyl, and —OCH₃.

36. The compound, tautomer, solvate, stereoisomer, or pharmaceutically acceptable salt of claim 1, wherein R^{d}, for each occurrence, is independently selected from halogen, C₁-C₆ alkyl, NO₂, CN, and C₁-C₆ alkoxy, wherein the C₁-C₆ alkyl and C₁-C₆ alkoxy of R^{d} are each optionally substituted with 1 to 3 groups selected from halogen.

37. The compound, tautomer, solvate, stereoisomer, or pharmaceutically acceptable salt of claim 1, wherein R^{d} is selected from F, methyl, Cl, NO₂, and —OCH₃.

38. The compound, tautomer, solvate, stereoisomer, or pharmaceutically acceptable salt of claim 1, wherein R^{d} is selected from halogen, C₁-C₂ alkyl, and C₁-C₂ alkoxy.

39. The compound, tautomer, solvate, stereoisomer, or pharmaceutically acceptable salt claim 1, wherein R^{d} is selected from F, methyl, and —OCH₃.

40. The compound, tautomer, solvate, stereoisomer, or pharmaceutically acceptable salt of claim 1, wherein R^{e}, for each occurrence, is independently selected from halogen, cyano, C₁1-C₁₀ alkyl, phenyl, 5- to 7-membered heteroaryl, 3- to 10-membered heterocyclyl, C₃-C₁₀ cycloalkyl, —C(═O)R^{s}, C₂-C₁₀ alkenyl, ═O, ═NR^{p}; —C(═O)OR^{s}, —C(═O)NR^{p}R^{q}, —NR^{p}R^{q}, and —NR^{p}C(═O)R^{s}, wherein
the C₁-C₁₀ alkyl and C₂-C₁₀ alkenyl of R^{e} are each optionally substituted with 1 to 3 groups selected from 5- to 7-membered heteroaryl, C₃-C₁₀ cycloalkyl, 3- to 10-membered heterocyclyl, —OR^{s}, —C(═O)NR^{p}R^{q}, —NR^{p}C(═O)NR^{g}R^{r}, and —NR^{p}R^{q};
the 3- to 10-membered heterocyclyl and C₃-C₁₀ cycloalkyl of R^{e} and the 3- to 10-membered heterocyclyl and C₃-C₁₀ cycloalkyl of the C₁-C₁₀ alkyl and C₂-C₁₀ alkenyl of R^{e}, are each optionally substituted with 1 to 3 groups selected from ═O and C₁-C₆ alkyl optionally substituted with 1 to 3 groups selected from halogen and —OR^{s};
the phenyl and 5- to 7-membered heteroaryl of R^{e} and the 5- to 7-membered heteroaryl of the C₁-C₁₀ alkyl and C₂-C₁₀ alkenyl of R^{e} are each optionally substituted with 1 to 3 groups selected from C₁-C₆ alkyl optionally substituted with 1 to 3 groups selected from halogen and —OR^{s};
R^{p}, R^{q}, and R^{r}, for each occurrence, are independently selected from hydrogen, C₃-C₁₀ cycloalkyl, and C₁-C₁₀ alkyl, wherein the C₃-C₁₀ cycloalkyl and C₁-C₁₀ alkyl of R^{p}, R^{q}, and R^{r} are each optionally substituted with 1 to 3 groups selected from halogen, —OR^{s} and —C(═O)OR^{s};
R^{s}, for each occurrence, is independently selected from H, C₃-C₁₀ cycloalkyl, 3- to 10-membered heterocyclyl, and C₁-C₁₀ alkyl optionally substituted with 1 to 3 groups selected from halogen, C₃-C₁₀ cycloalkyl, 3- to 10-membered heterocyclyl, and C₁-C₆ alkoxy, wherein
the C₃-C₁₀ cycloalkyl and 3- to 10-membered heterocyclyl of R^{s} and the C₃-C₁₀ cycloalkyl and 3- to 10-membered heterocyclyl of the C₁-C₁₀ alkyl of R^{s} are each optionally substituted with 1 to 3 groups of C₁-C₆ alkyl optionally substituted by halogen, and the C₁-C₆ alkoxy of the C₁-C₁₀ alkyl of R^{s} is optionally substituted with 1 to 3 groups of halogen.

41. The compound, tautomer, solvate, stereoisomer, or pharmaceutically acceptable salt of claim 1, wherein R^{e}, for each occurrence, is selected from methyl, ethyl, ═O, —(CH₂)₃OCH₃, —(CH₂)₂OCH₃, —CH₂C(═O)NH₂, —(CH₂)₂C(═O)NH₂, —C(═O)C(CH₃)₃, —C(═O)NHC(CH₃)₃, —(CH₂)₂NHC(═O)NH₂, —(CH₂)₃NHCH₃, —(CH₂)₂NHCH₃, —(CH₂)₃C(═O)NH₂, —(CH₂)₂N(CH₃)₂, —(CH₂)₂OH, 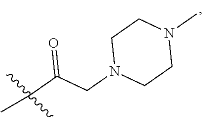 [SMILES: *C(=O)CN1CCN(C)CC1]
—C(═O)CH₃, —(CH₂)₂NH₂, 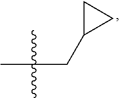 [SMILES: *CC1CC1]
—C(═O)OC(CH₃)₃, 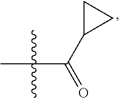 [SMILES: *C(=O)C1CC1]
—C(CH₃)₃, —(CH₂)₂O(CH₂)₂OCH₃, Bn (benzyl), —(CH₂)₃N(CH₃)₂, 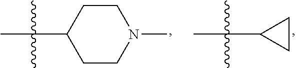
—CH(CH₃)₂, 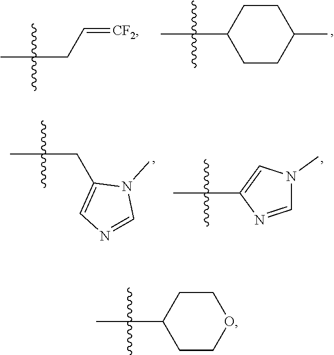
═NH, ═NCH₃, —C(═O)OH, —C(═O)CF₃, —C(═O)NHCH₂CH₃, —NHC(═O)CH₃, 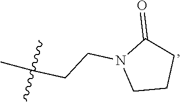 [SMILES: *CCN1CCCC1=O]
—N(CH₃)(CH₂)₂OCH₂OCH₂CH₃, —NH₂, —C(═O)NH₂, 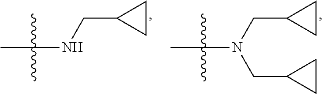 [SMILES: *NCC1CC1 ; *N(CC1CC1)CC1CC1]
—N(CH₃)₂, —NHCH₂CH₃, and

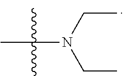 [SMILES: *N(CC)CC]

42. The compound, tautomer, solvate, stereoisomer, or pharmaceutically acceptable salt of claim 1, wherein R^{e} is selected from ═O, —C(═O)NR^{p}R^{q}, and C₁-C₆ alkyl, wherein the C₁-C₆ alkyl is optionally substituted with 1 to 3 groups selected from C₁-C₃ alkoxy, —C(═O)NR^{p}R^{q}, and —NR^{p}R^{q}, wherein R^{p} and R^{q} are each selected from H and C₁-C₃ alkyl.

43. The compound, tautomer, solvate, stereoisomer, or pharmaceutically acceptable salt of claim 1, wherein R^{e} is selected from C₁-C₂ alkyl, ═O, and —C(═O)NH₂.

44. The compound, tautomer, solvate, stereoisomer, or pharmaceutically acceptable salt of claim 1, wherein R^{a} and R^{b} join to form a 5- to 6-membered carbocyclic, heterocyclic, aromatic, or heteroaromatic ring optionally substituted with 1 to 3 groups selected from ═O, C₁-C₆ alkyl, and 5- to 7-membered heterocyclyl, wherein the C₁-C₆ alkyl is optionally substituted with 1 to 3 groups of halogen, and the 5- to 7-membered heterocyclyl is optionally substituted with 1 to 3 groups of C₁-C₃ alkyl optionally substituted with 1 to 3 groups of halogen.

45. The compound, tautomer, solvate, stereoisomer, or pharmaceutically acceptable salt of claim 1, wherein R^{a} and R^{b} join to form a structure selected from:

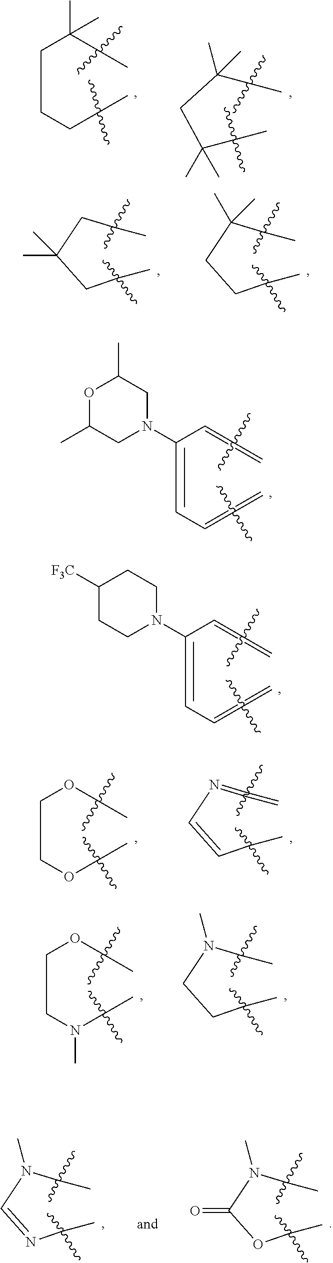

46. The compound of claim 1, wherein the compound has one of the following structural Formulae VIIIa-1, VIIId-1, and VIIIf-1: 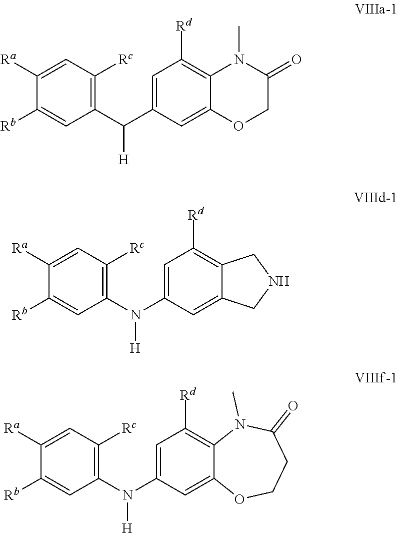
a tautomer thereof, a solvate or stereoisomer of the compound or the tautomer, or a pharmaceutically acceptable salt of the foregoing, wherein:
R^{a} is selected from 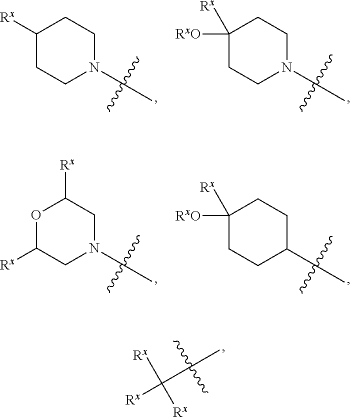
—OR^{x}, and —CH₂R^{x}, wherein R^{x}, for each occurrence, is independently selected from CF₃ and C₁ to C₂ alkyl;
R^{b} is selected from H, halogen, C₁ to C₂ alkyl, and C₁ to C₂ alkoxy;
R^{c} is selected from halogen, C₁ to C₂ alkyl, and C₁ to C₂ alkoxy; and
R^{d} is selected from halogen, C₁ to C₂ alkyl, and C₁ to C₂ alkoxy.

47. The compound according to claim 1, wherein the compound is selected from: 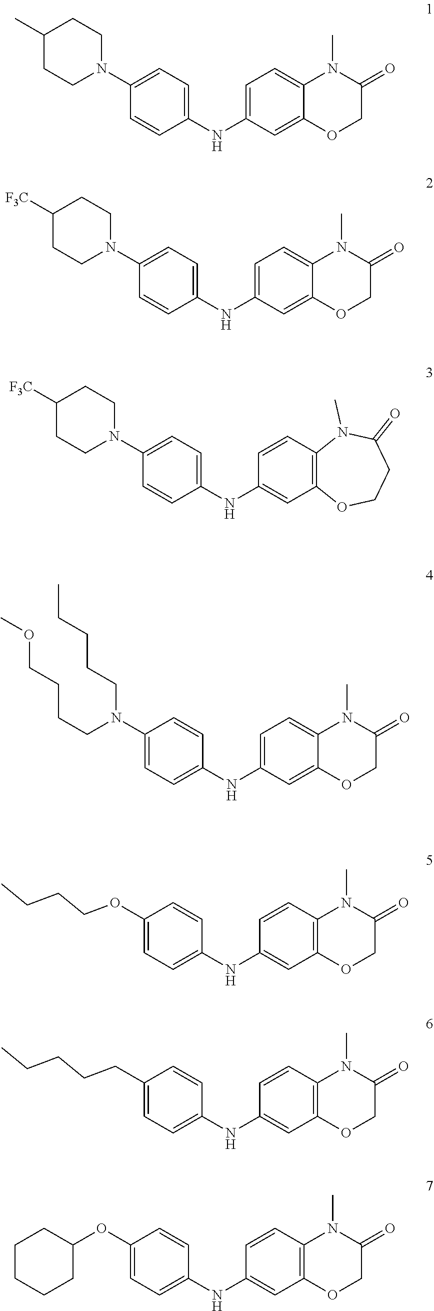 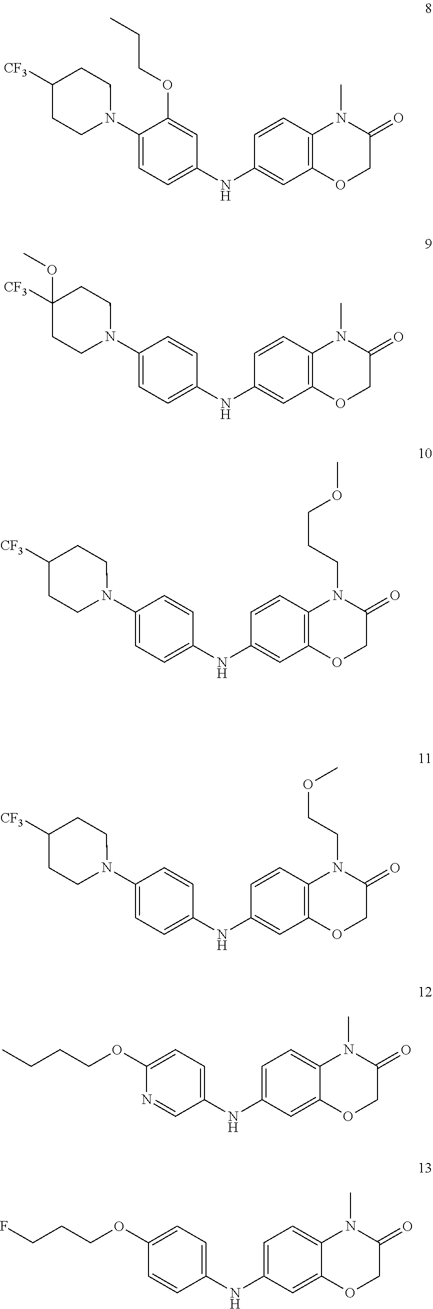 [SMILES: CCCOc1cc(Nc2ccc3c(c2)OCC(=O)N3C)ccc1N1CCC(C(F)(F)F)CC1 ; COC1(C(F)(F)F)CCN(c2ccc(Nc3ccc4c(c3)OCC(=O)N4C)cc2)CC1 ; COCCCN1C(=O)COc2cc(Nc3ccc(N4CCC(C(F)(F)F)CC4)cc3)ccc21 ; COCCN1C(=O)COc2cc(Nc3ccc(N4CCC(C(F)(F)F)CC4)cc3)ccc21 ; CCCCOc1ccc(Nc2ccc3c(c2)OCC(=O)N3C)cn1 ; CN1C(=O)COc2cc(Nc3ccc(OCCCF)cc3)ccc21] 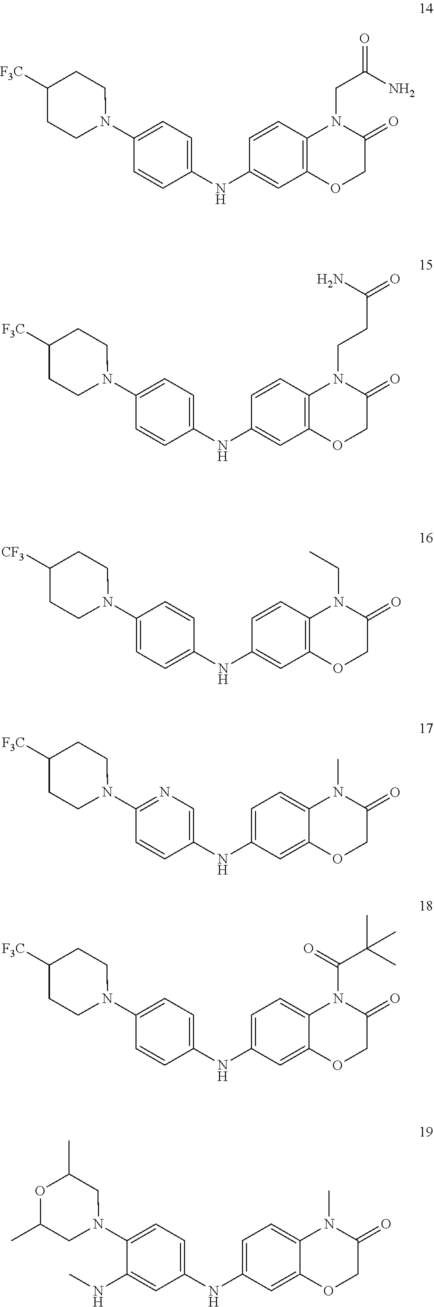 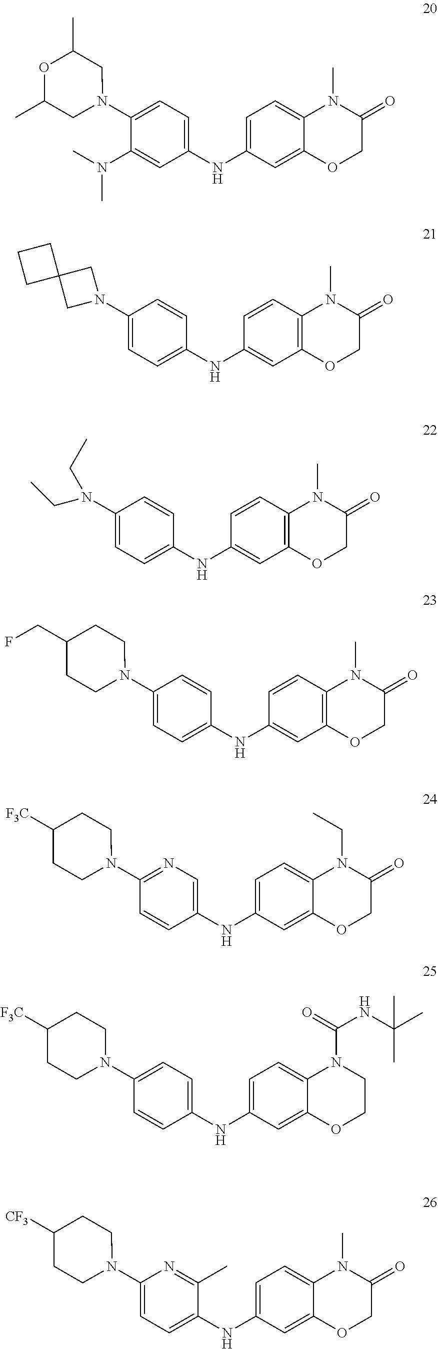 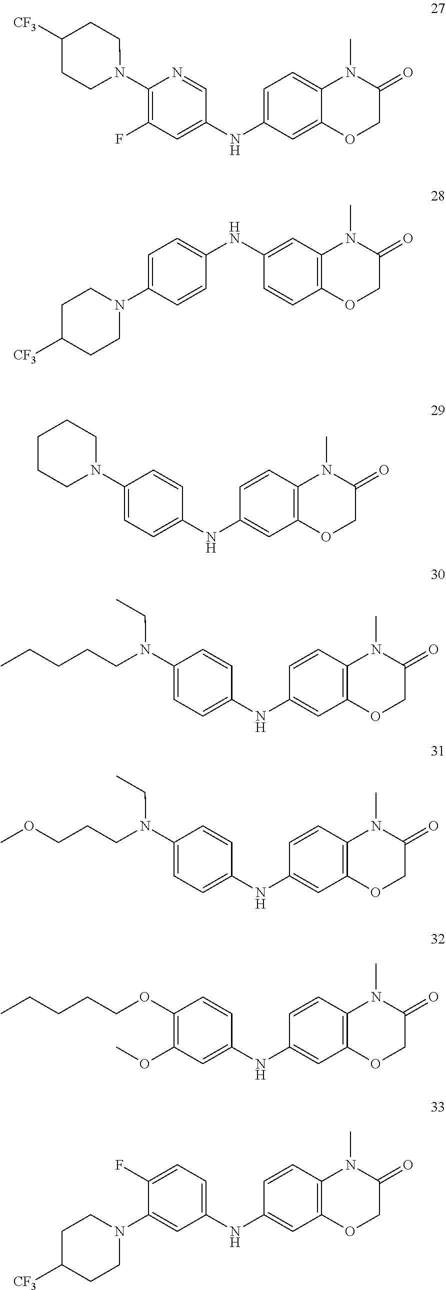 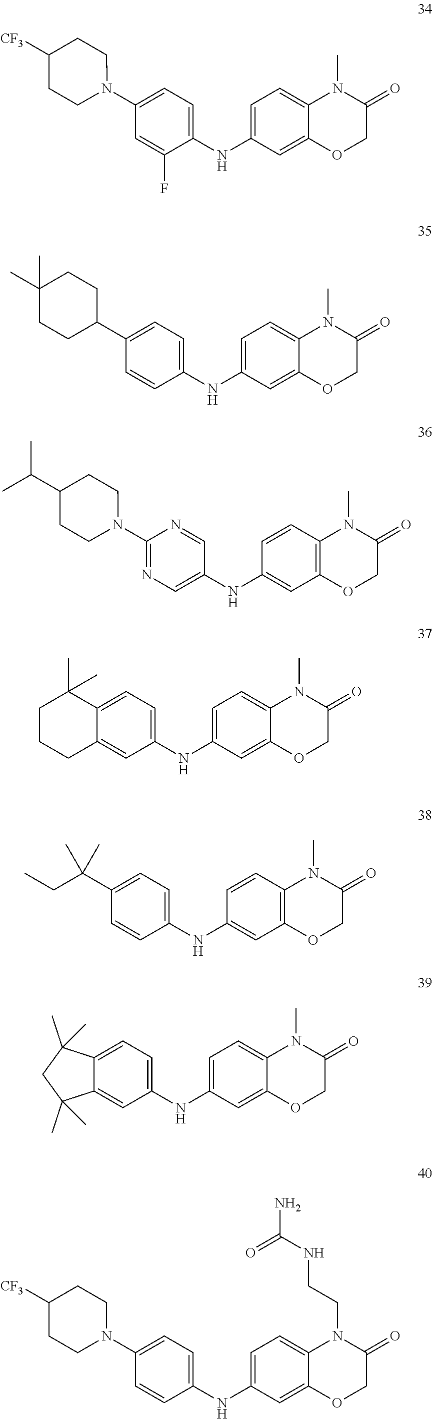 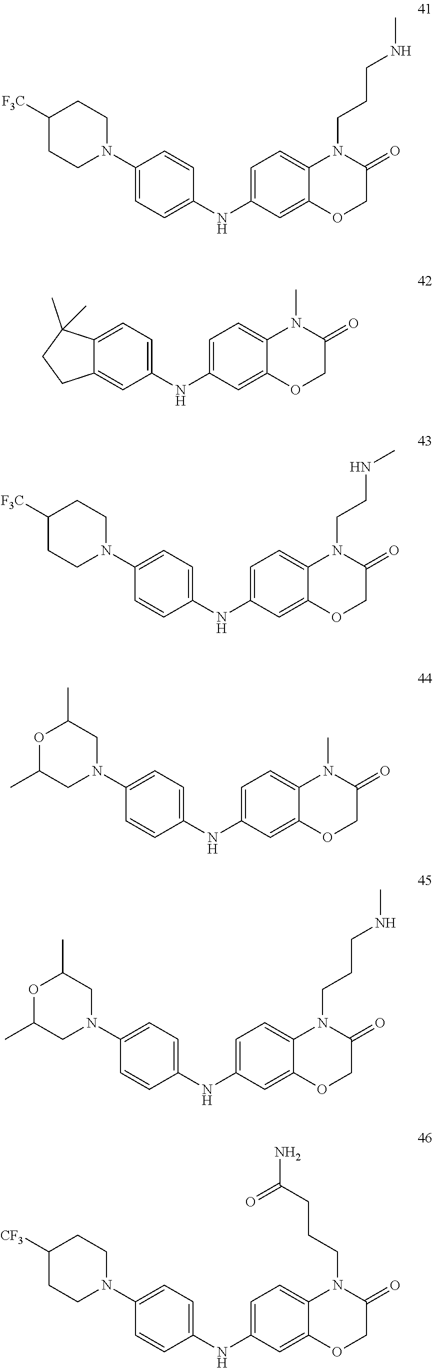 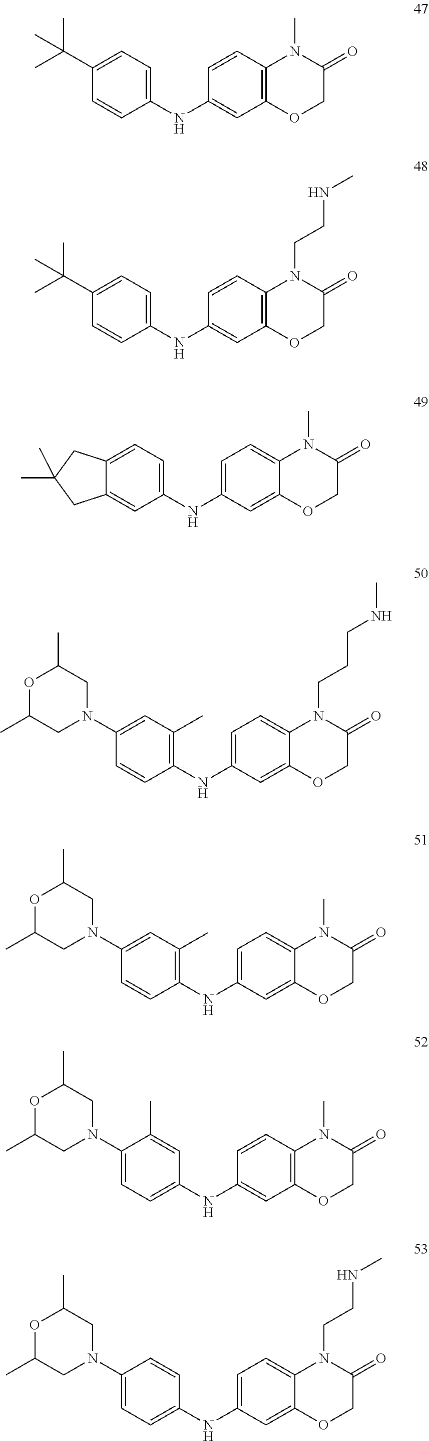 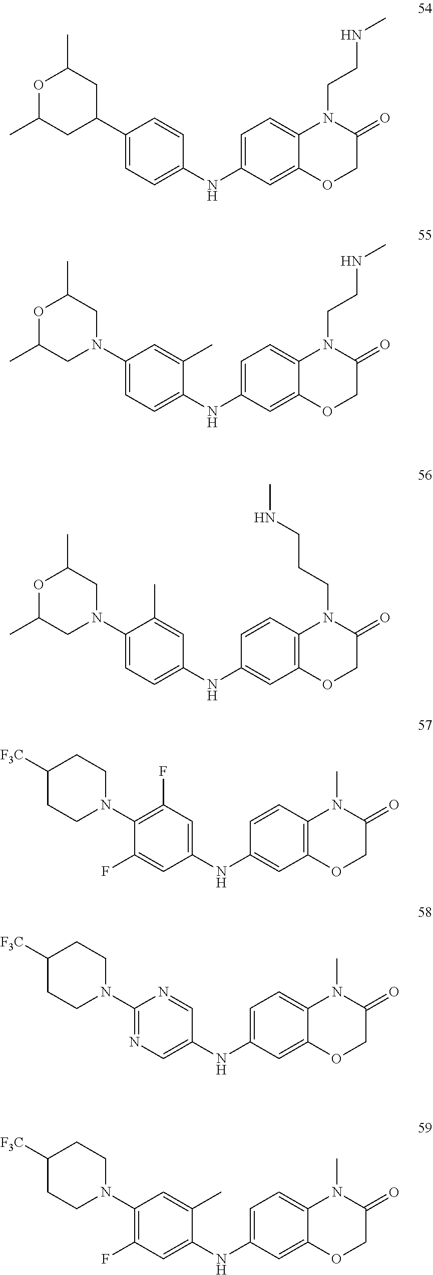 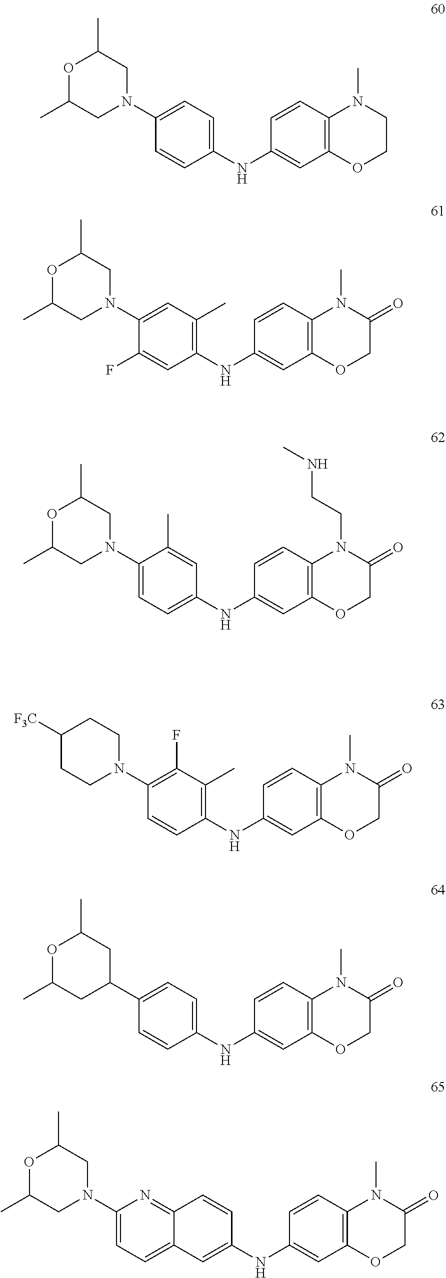 [SMILES: CC1CN(c2ccc(Nc3ccc4c(c3)OCCN4C)cc2)CC(C)O1 ; Cc1cc(N2CC(C)OC(C)C2)c(F)cc1Nc1ccc2c(c1)OCC(=O)N2C ; CNCCN1C(=O)COc2cc(Nc3ccc(N4CC(C)OC(C)C4)c(C)c3)ccc21 ; Cc1c(Nc2ccc3c(c2)OCC(=O)N3C)ccc(N2CCC(C(F)(F)F)CC2)c1F ; CC1CC(c2ccc(Nc3ccc4c(c3)OCC(=O)N4C)cc2)CC(C)O1 ; CC1CN(c2ccc3cc(Nc4ccc5c(c4)OCC(=O)N5C)ccc3n2)CC(C)O1] 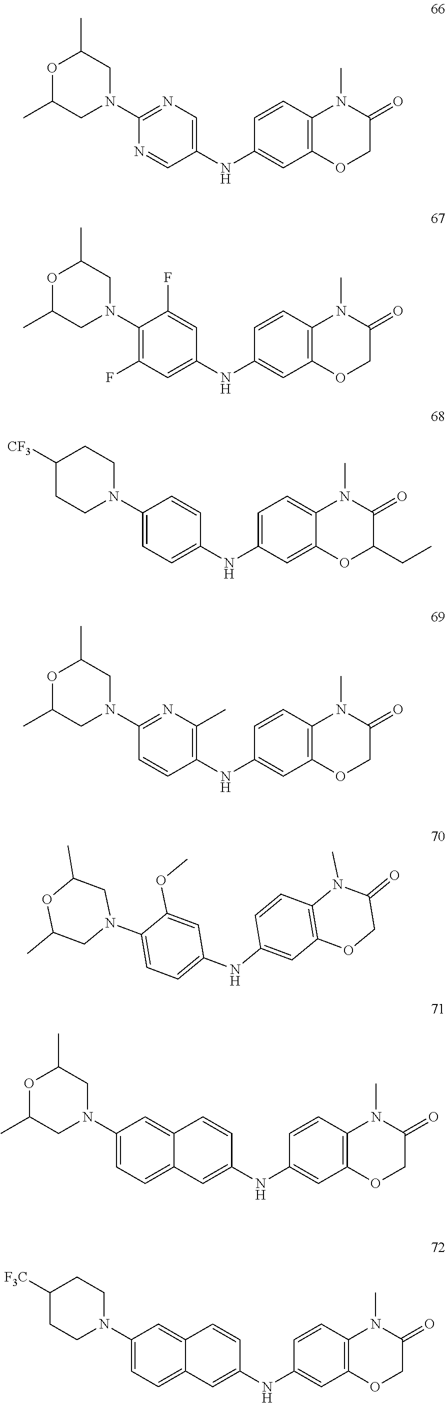 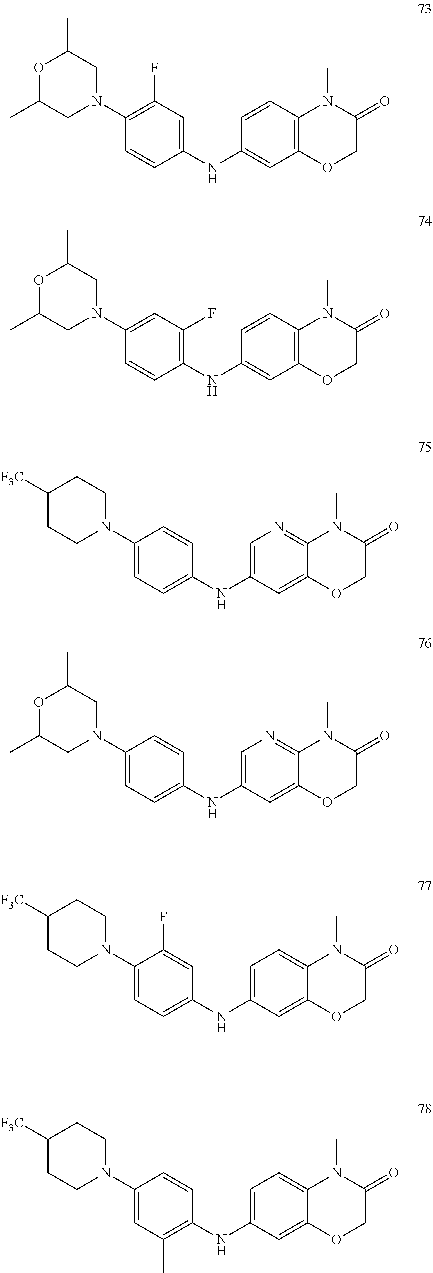 [SMILES: CC1CN(c2ccc(Nc3ccc4c(c3)OCC(=O)N4C)cc2F)CC(C)O1 ; CC1CN(c2ccc(Nc3ccc4c(c3)OCC(=O)N4C)c(F)c2)CC(C)O1 ; CN1C(=O)COc2cc(Nc3ccc(N4CCC(C(F)(F)F)CC4)cc3)cnc21 ; CC1CN(c2ccc(Nc3cnc4c(c3)OCC(=O)N4C)cc2)CC(C)O1 ; CN1C(=O)COc2cc(Nc3ccc(N4CCC(C(F)(F)F)CC4)c(F)c3)ccc21 ; Cc1cc(N2CCC(C(F)(F)F)CC2)ccc1Nc1ccc2c(c1)OCC(=O)N2C] 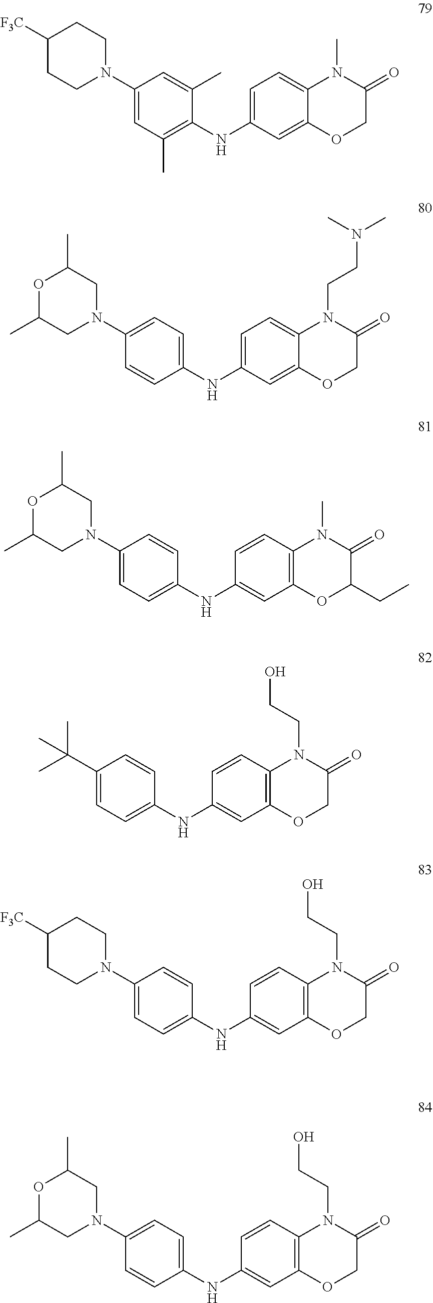 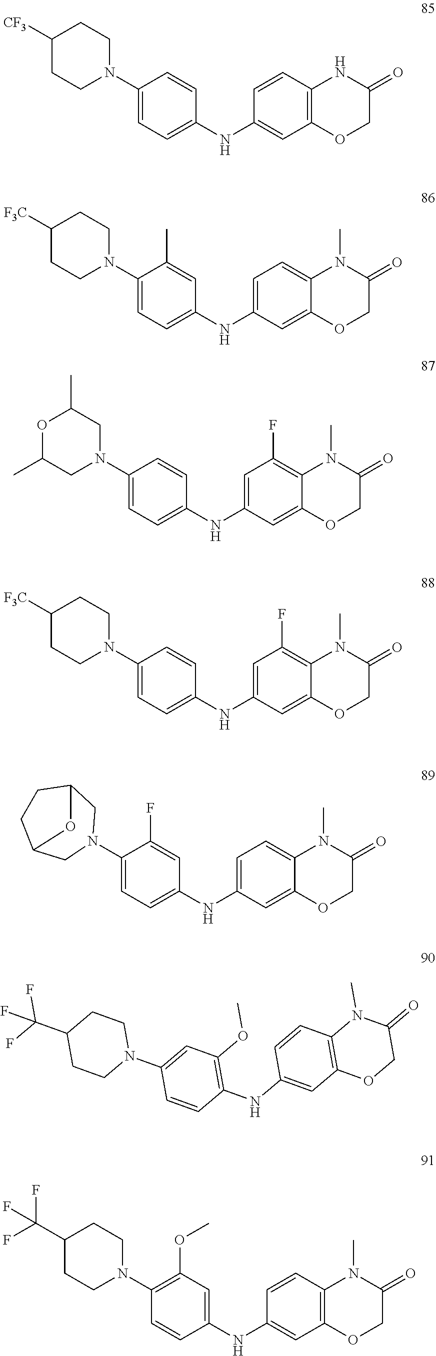 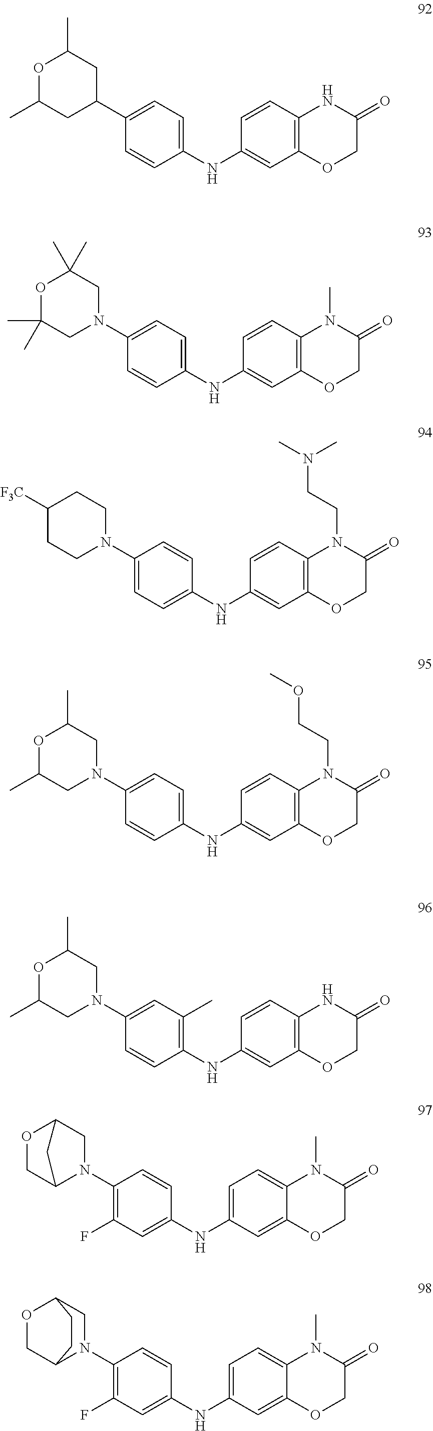 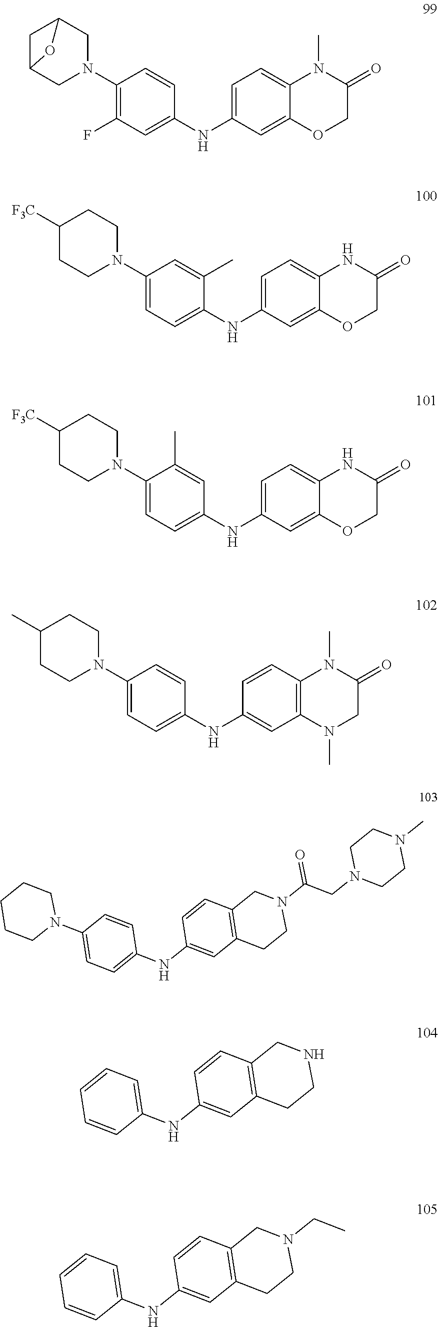 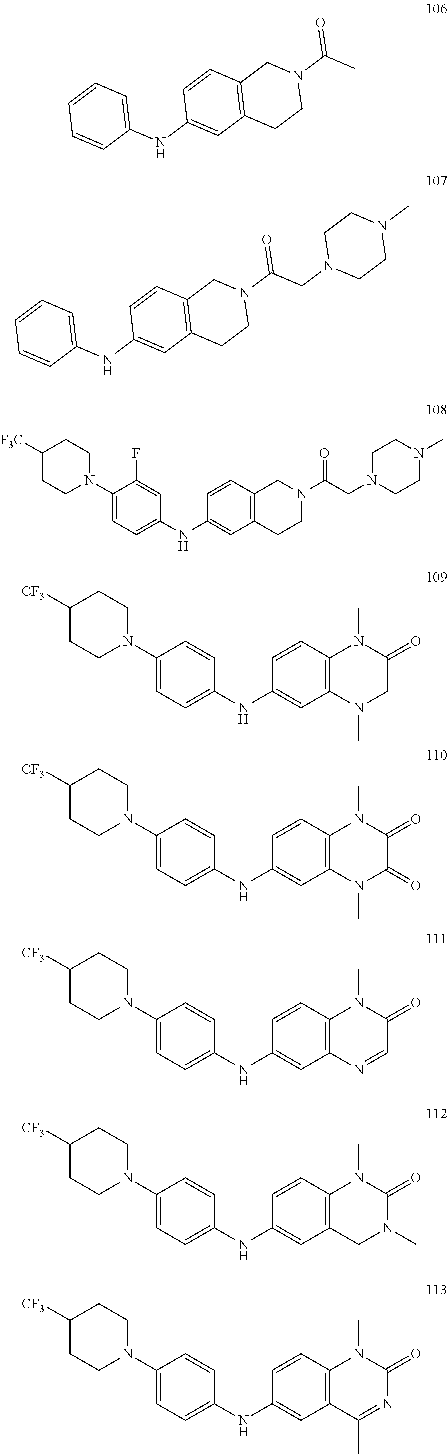 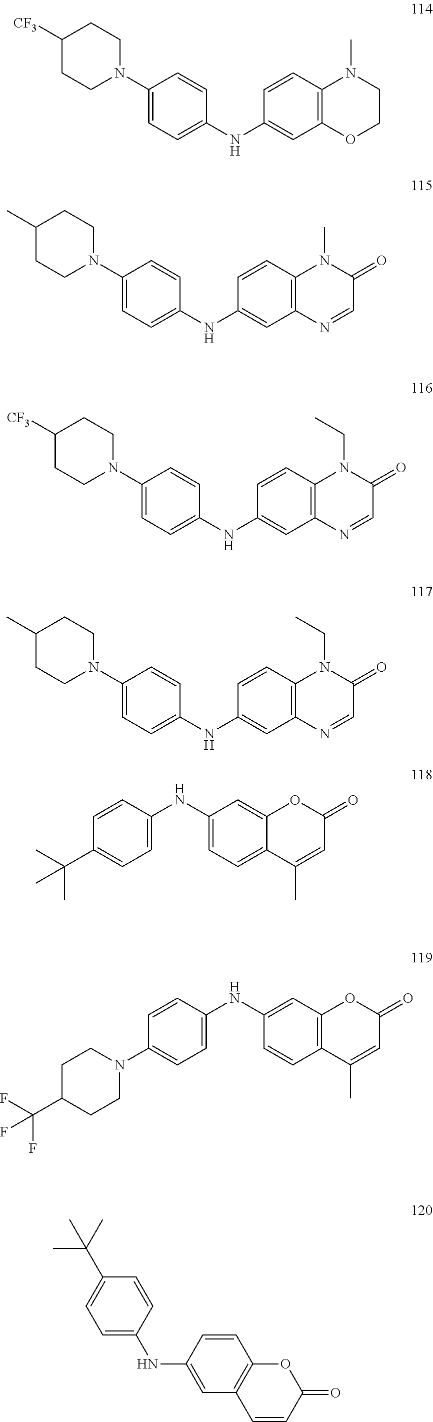 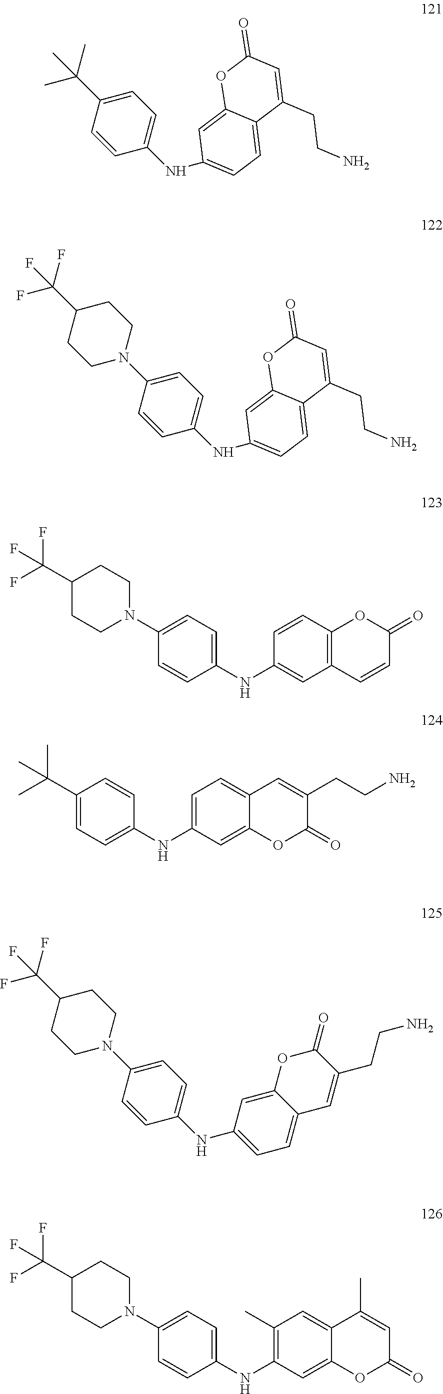 [SMILES: CC(C)(C)c1ccc(Nc2ccc3c(CCN)cc(=O)oc3c2)cc1 ; NCCc1cc(=O)oc2cc(Nc3ccc(N4CCC(C(F)(F)F)CC4)cc3)ccc12 ; O=c1ccc2cc(Nc3ccc(N4CCC(C(F)(F)F)CC4)cc3)ccc2o1 ; CC(C)(C)c1ccc(Nc2ccc3cc(CCN)c(=O)oc3c2)cc1 ; NCCc1cc2ccc(Nc3ccc(N4CCC(C(F)(F)F)CC4)cc3)cc2oc1=O ; Cc1cc2c(C)cc(=O)oc2cc1Nc1ccc(N2CCC(C(F)(F)F)CC2)cc1] 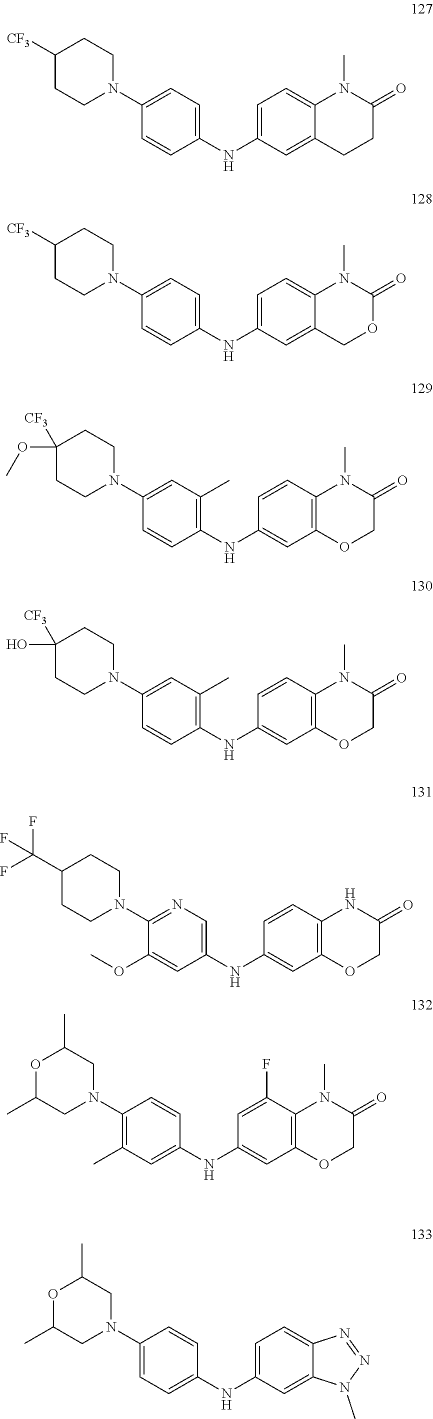 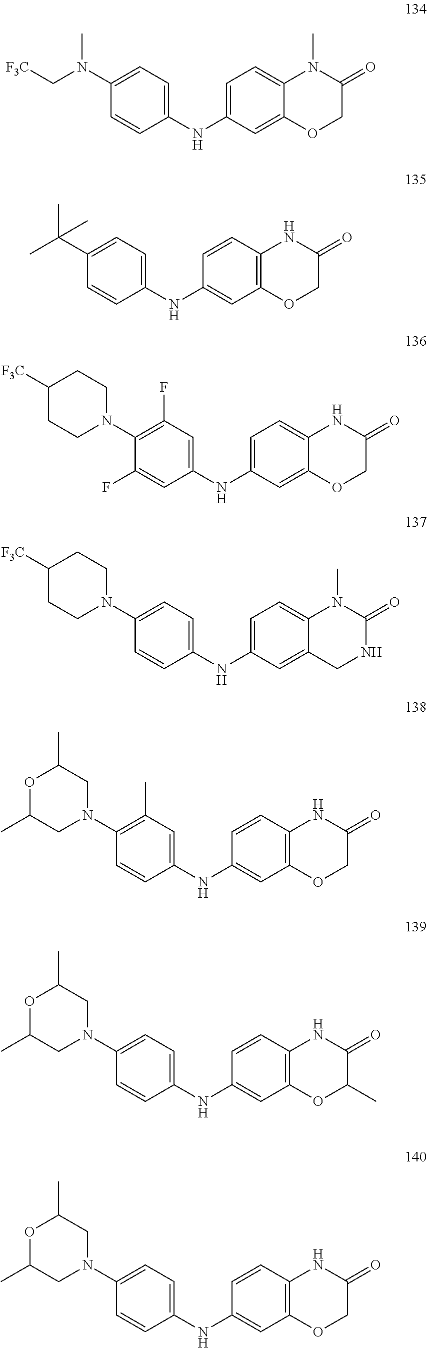 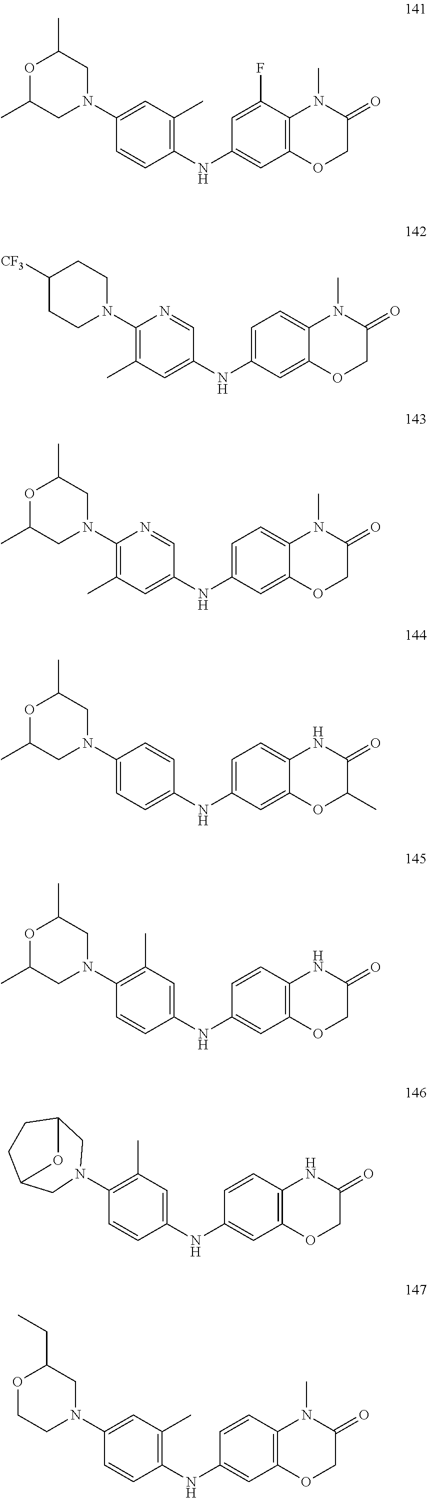 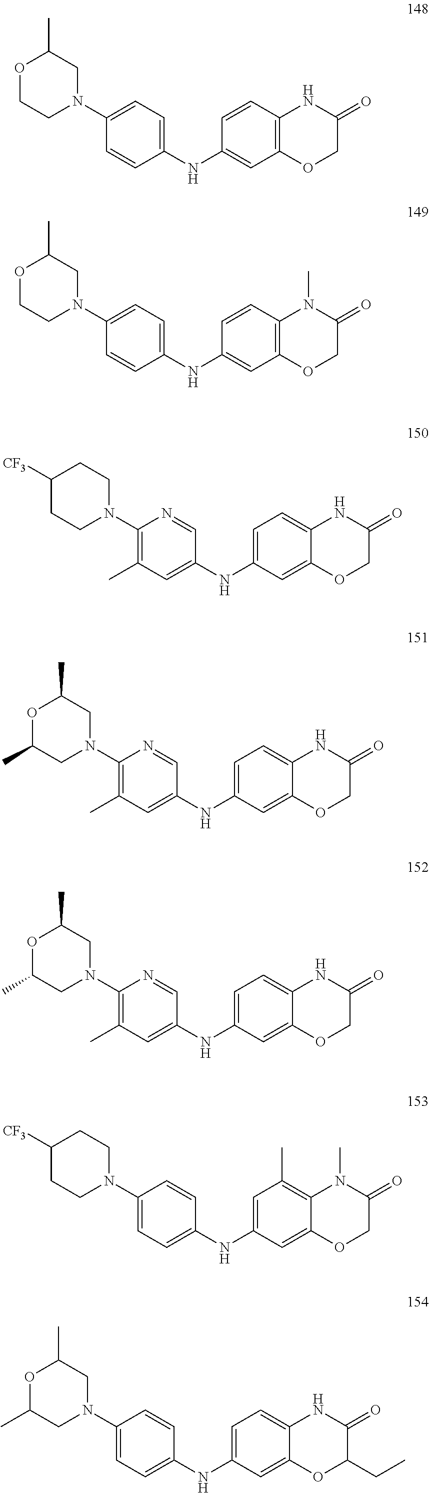 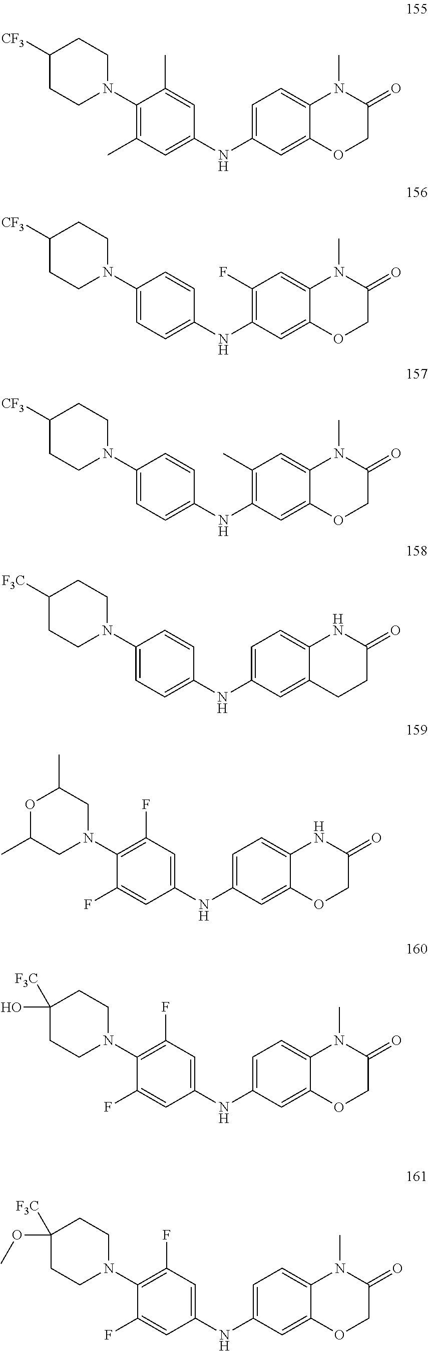 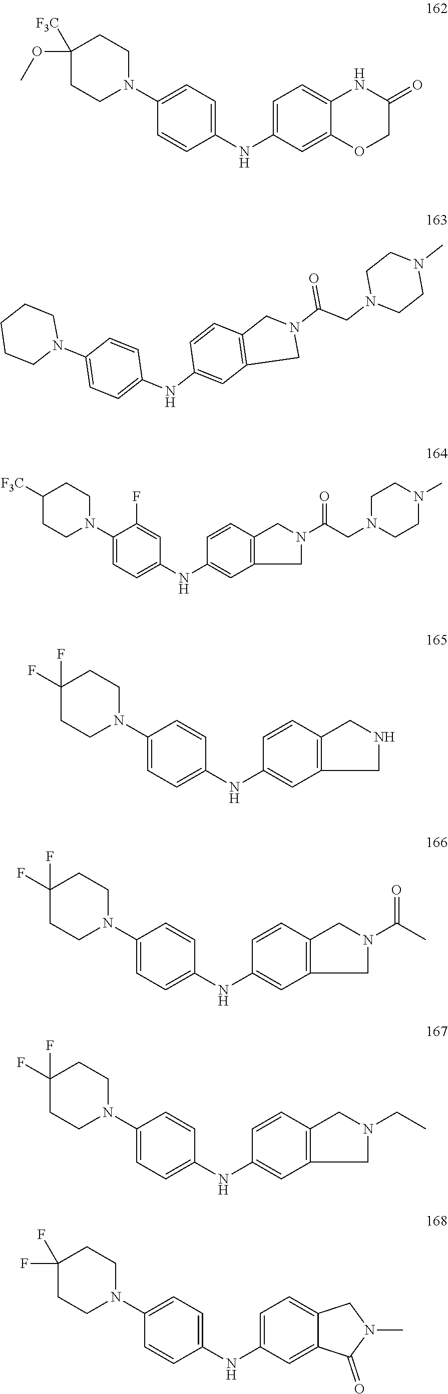 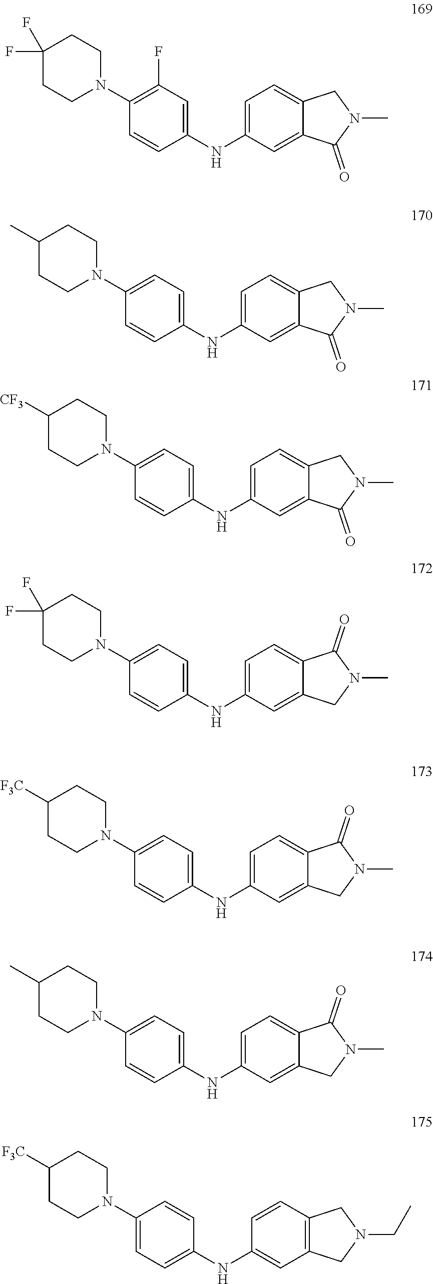 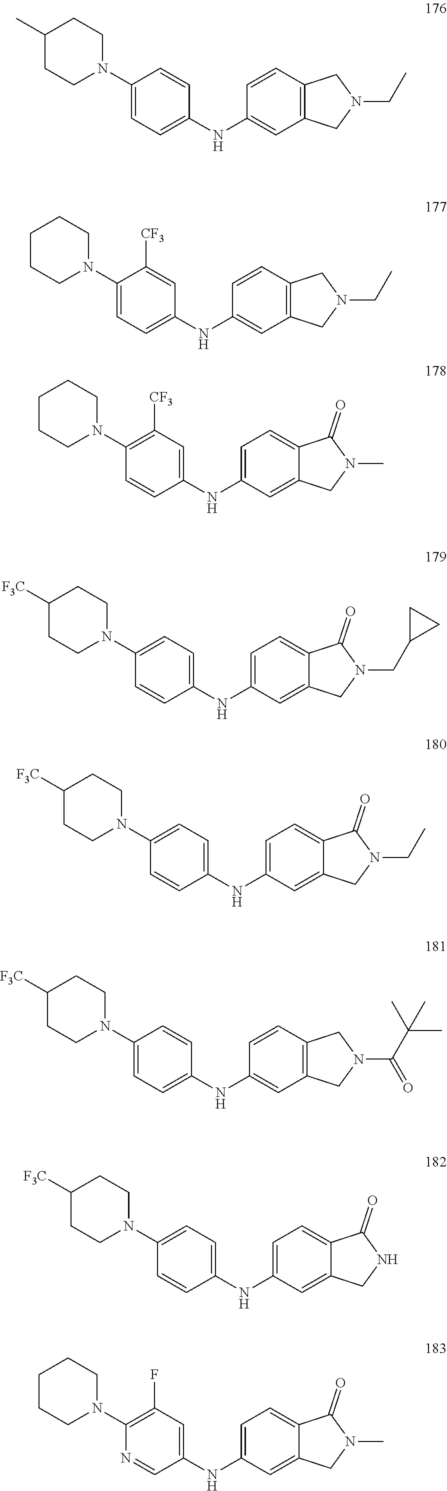 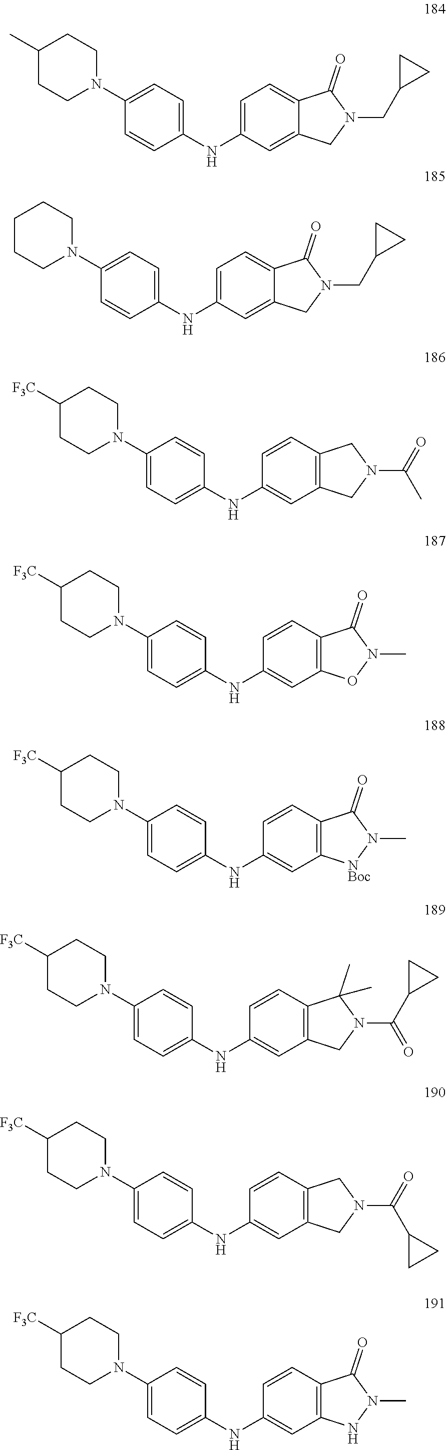 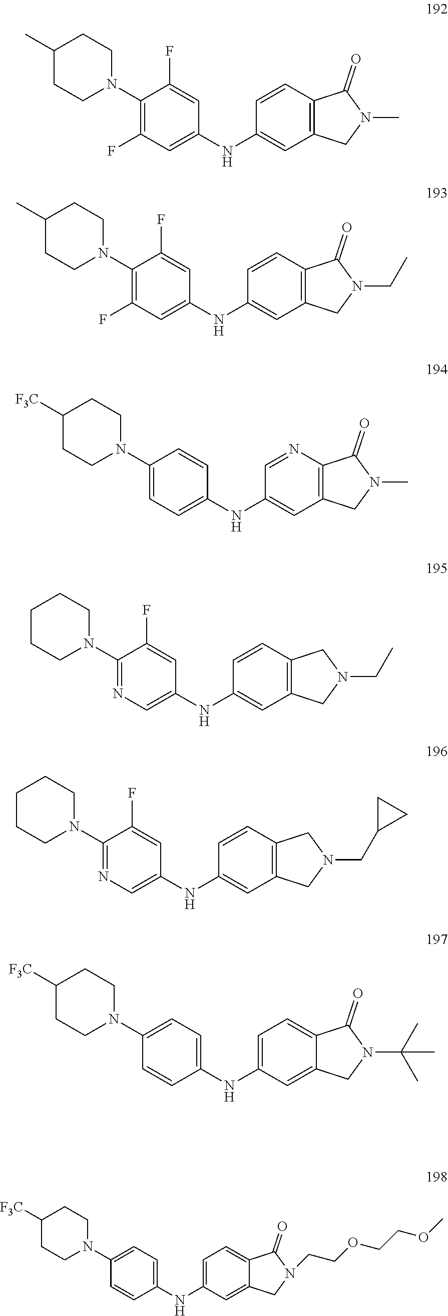 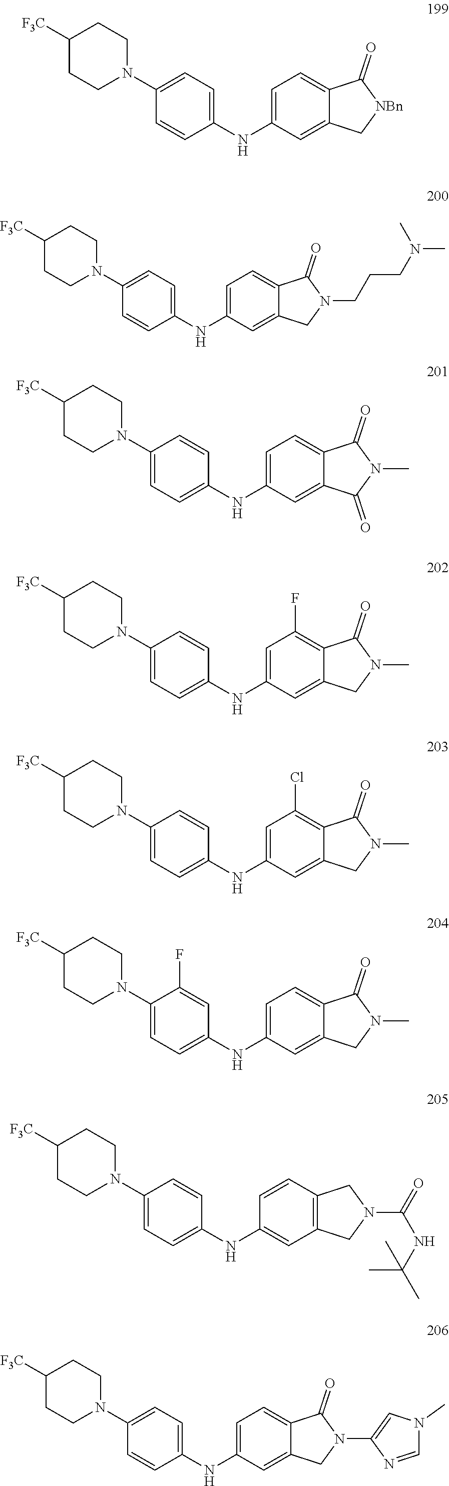 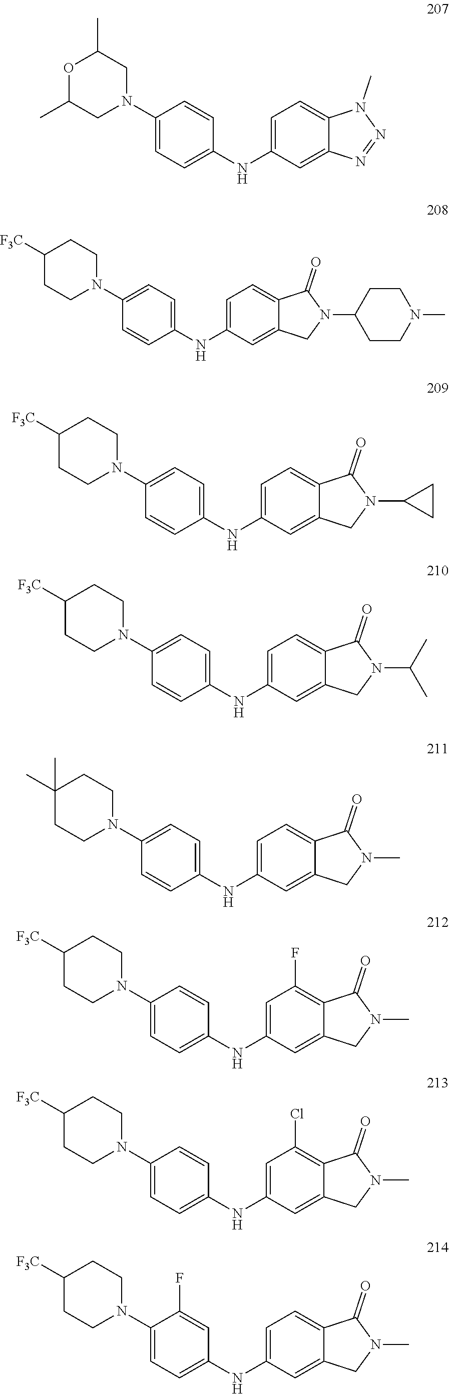 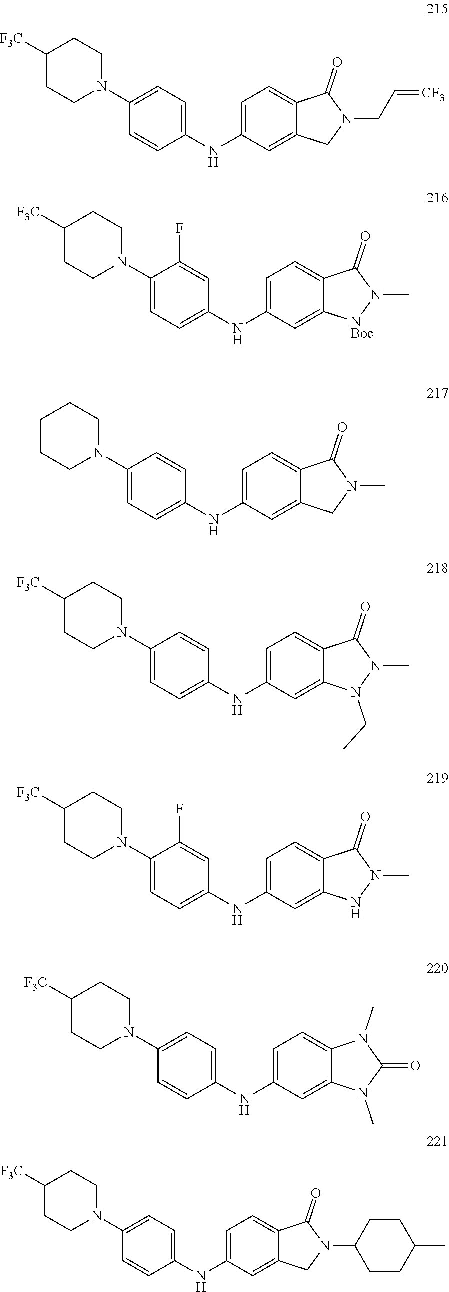 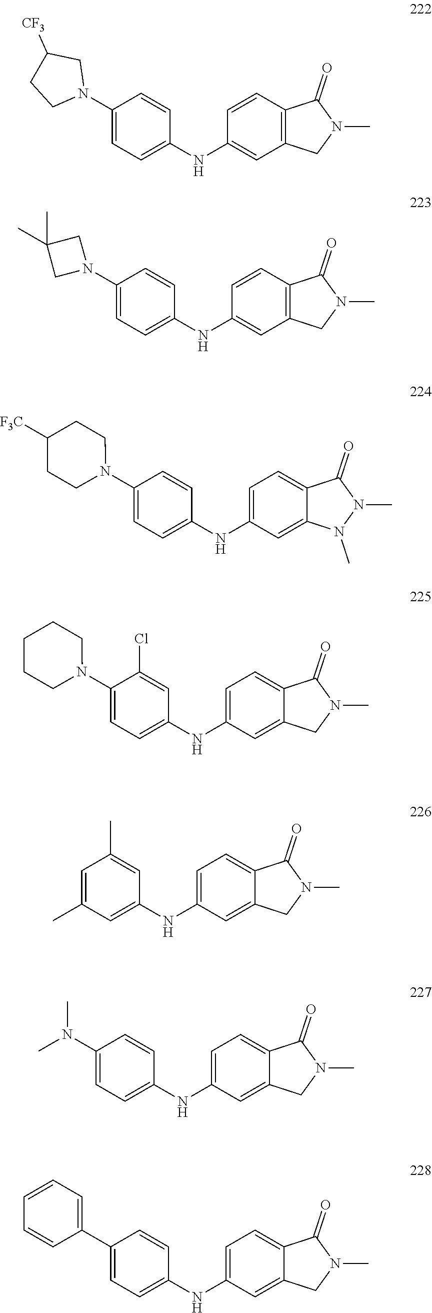 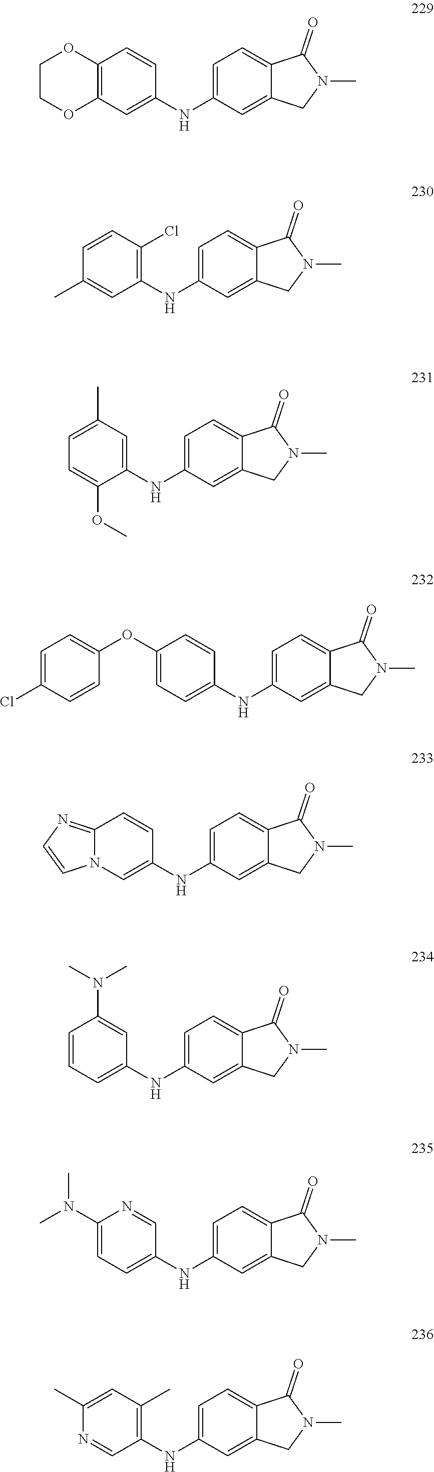 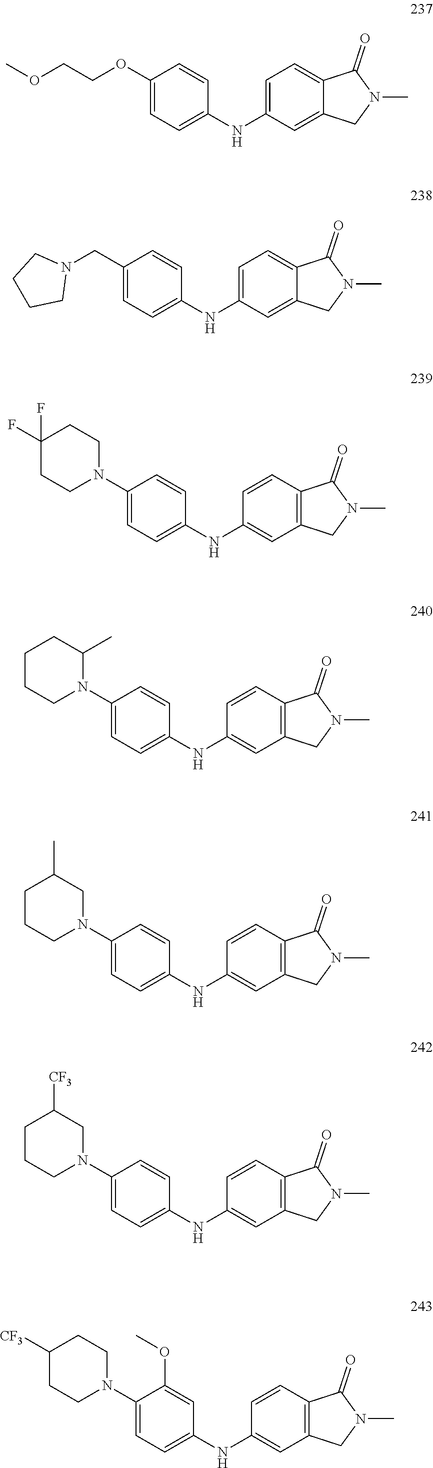 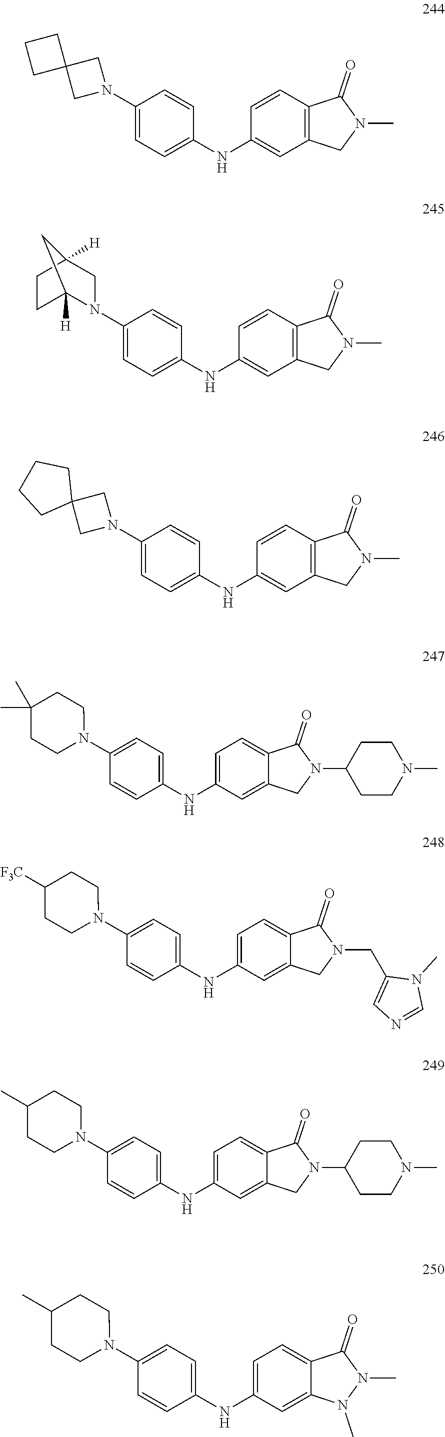 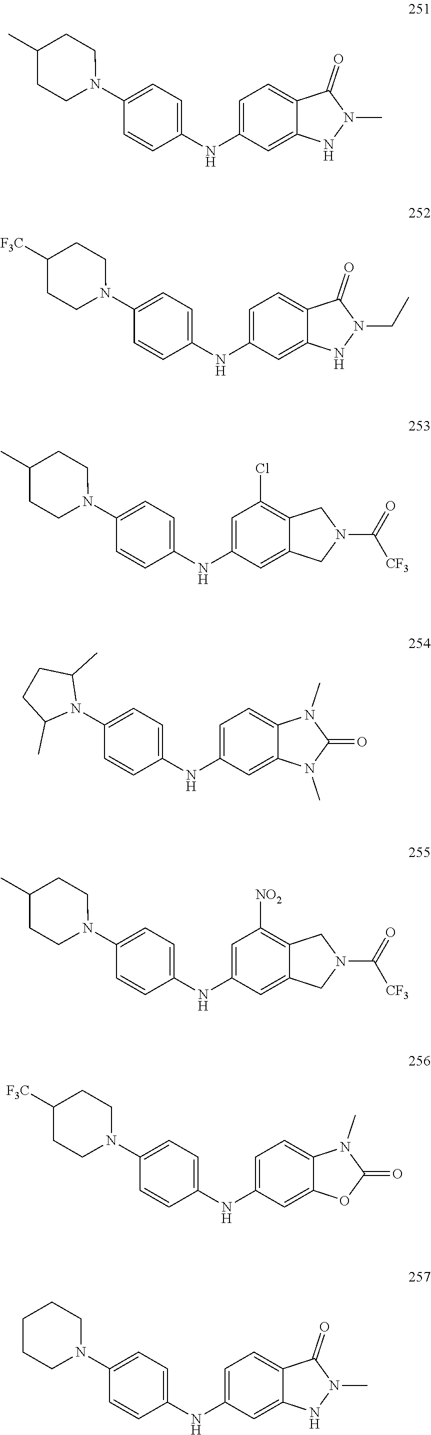 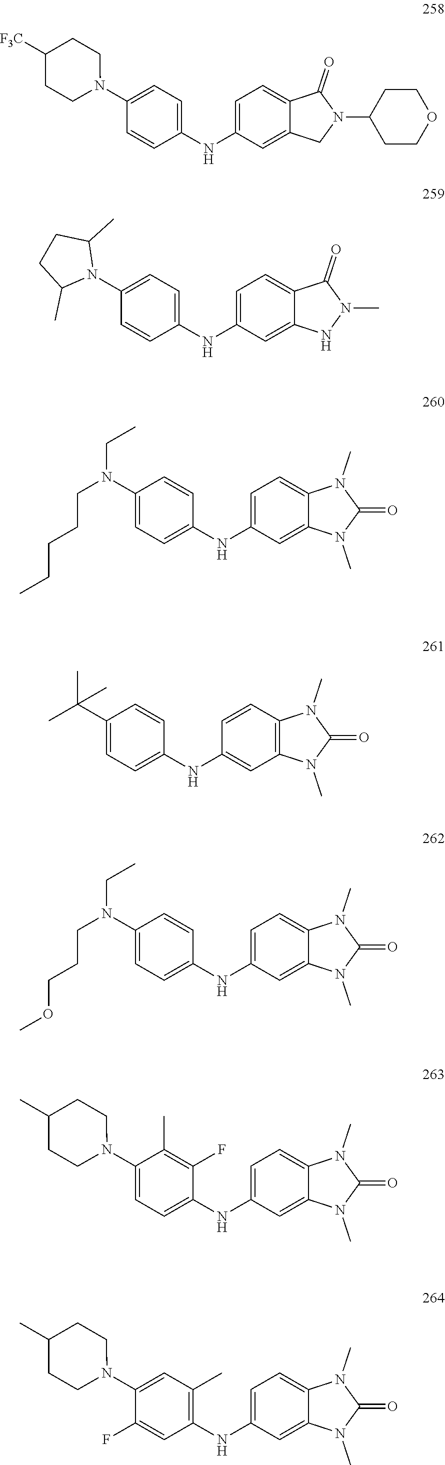 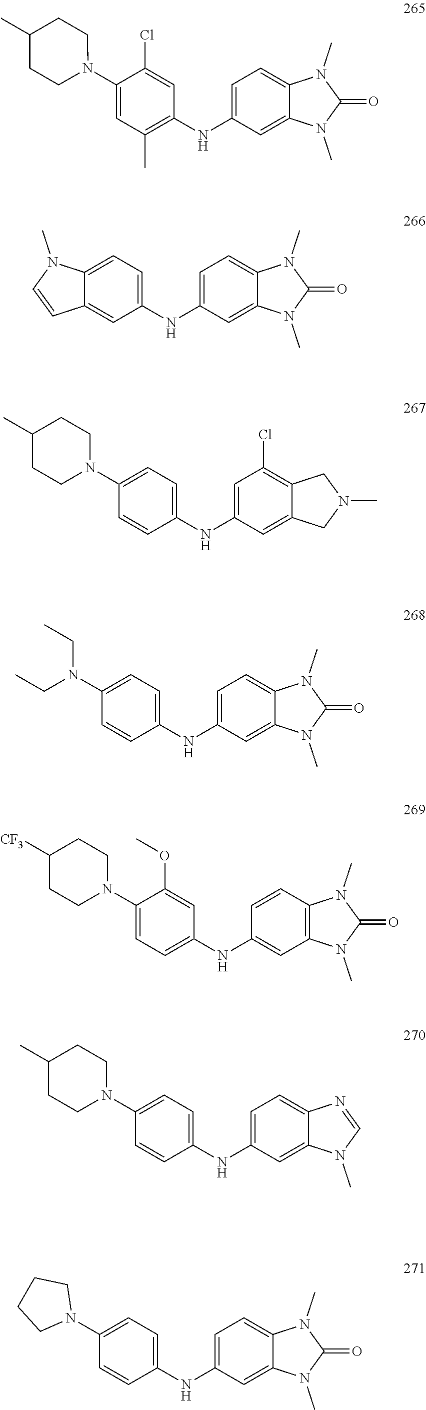 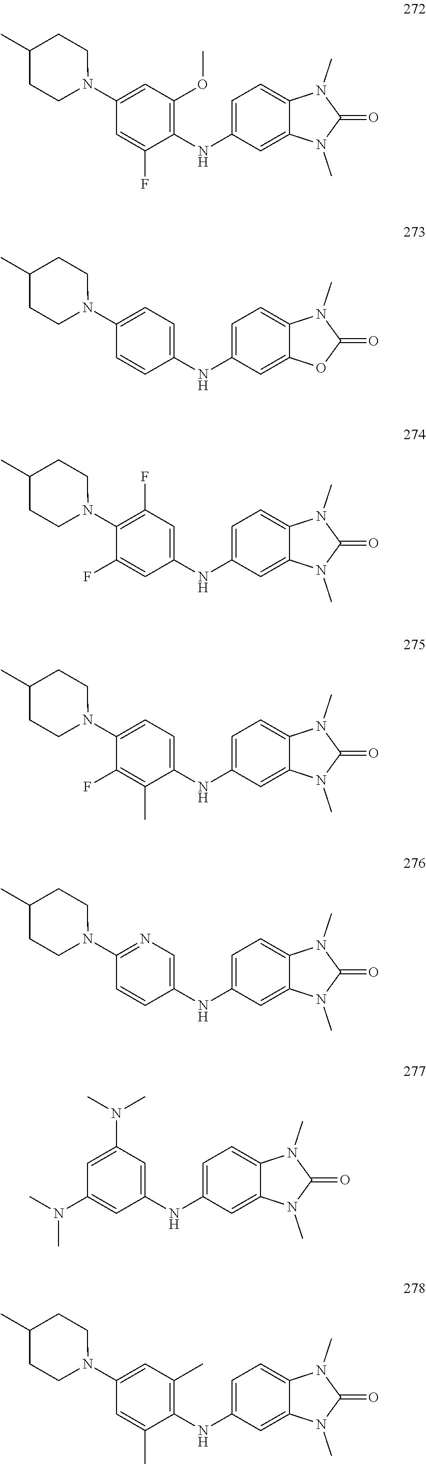 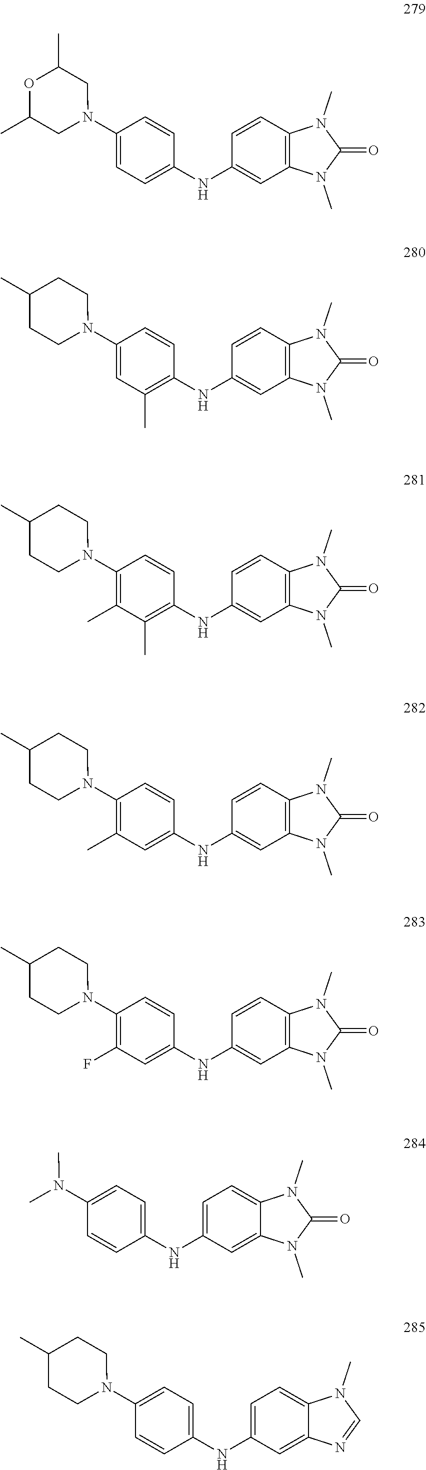 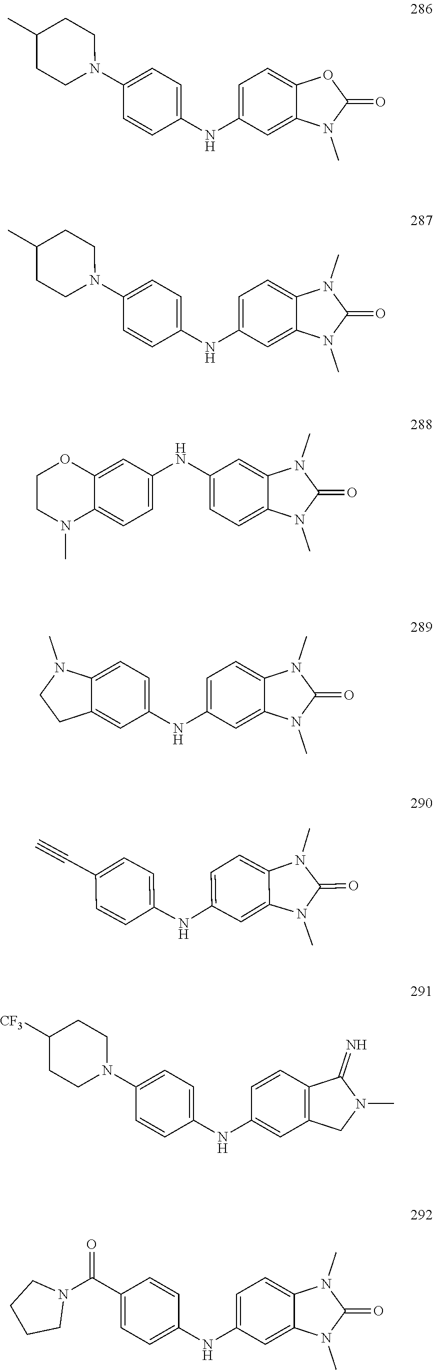 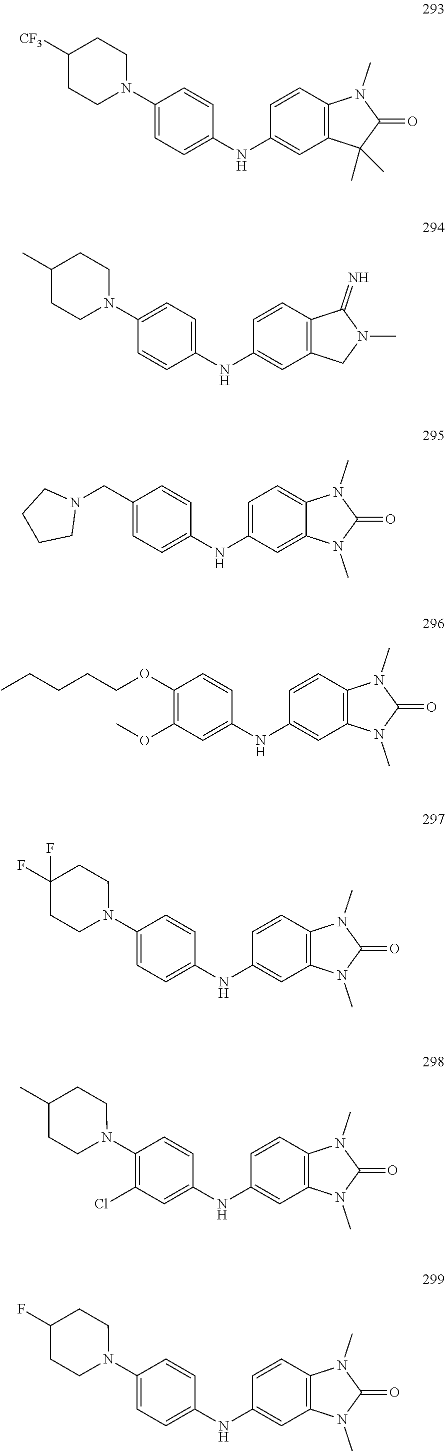 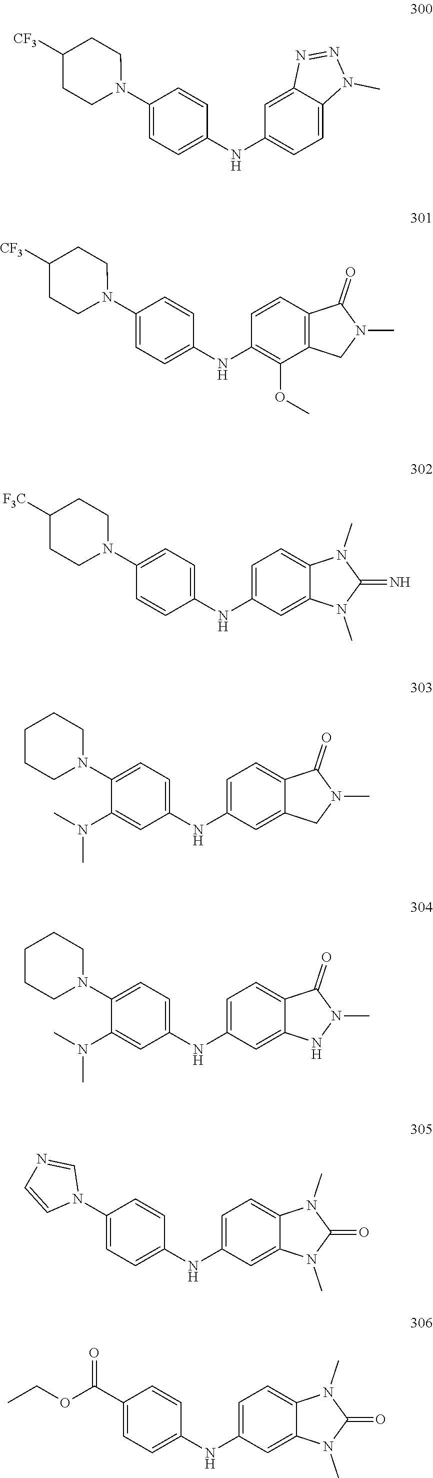 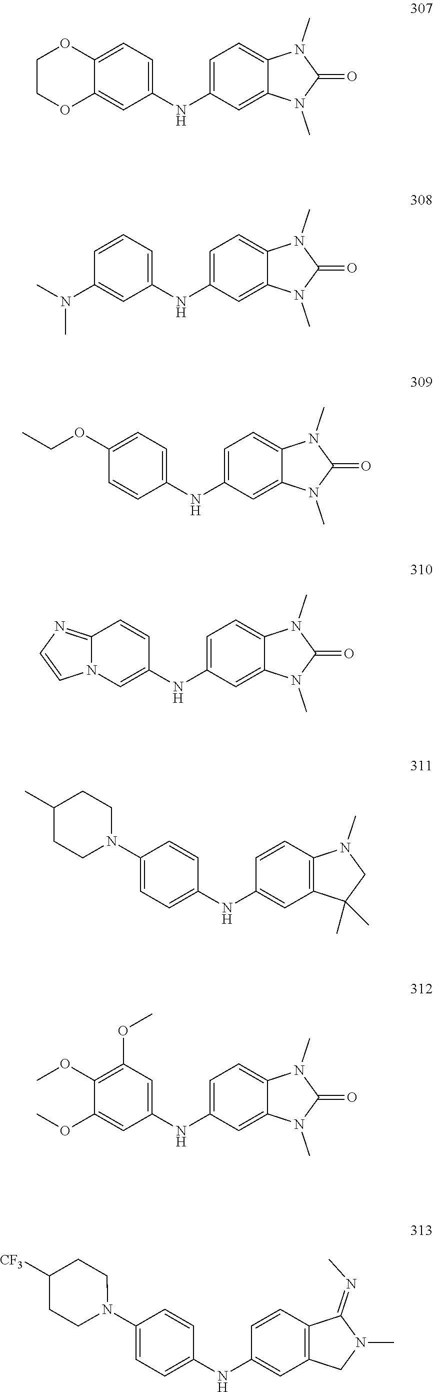 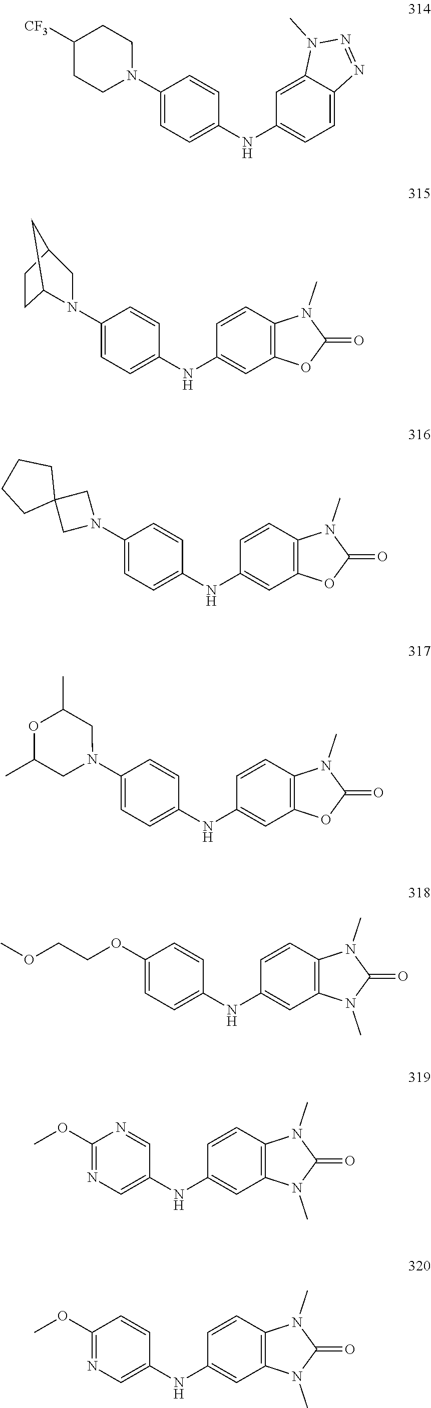 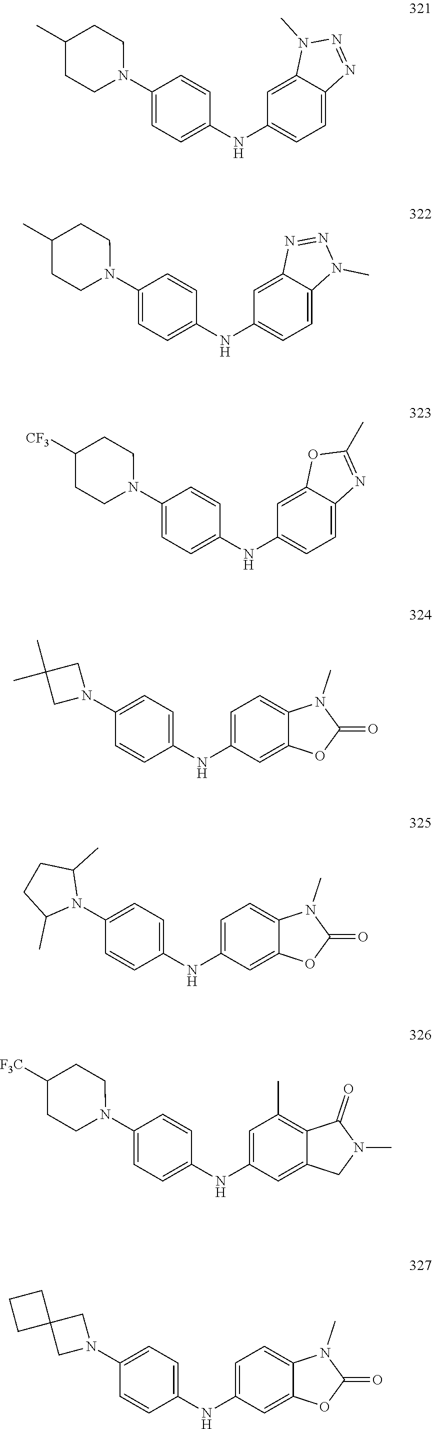 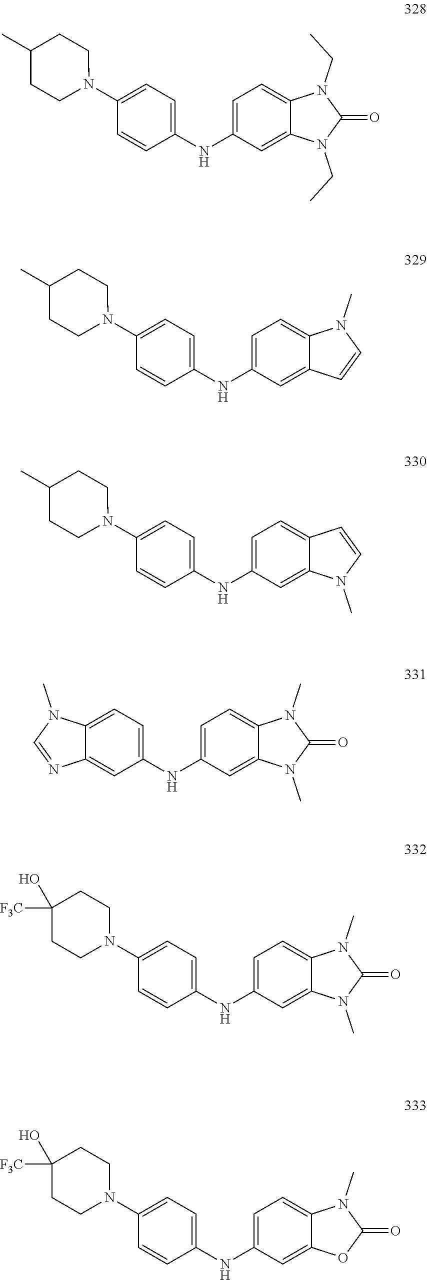 [SMILES: CCn1c(=O)n(CC)c2cc(Nc3ccc(N4CCC(C)CC4)cc3)ccc21 ; CC1CCN(c2ccc(Nc3ccc4c(ccn4C)c3)cc2)CC1 ; CC1CCN(c2ccc(Nc3ccc4ccn(C)c4c3)cc2)CC1 ; Cn1cnc2cc(Nc3ccc4c(c3)n(C)c(=O)n4C)ccc21 ; Cn1c(=O)n(C)c2cc(Nc3ccc(N4CCC(O)(C(F)(F)F)CC4)cc3)ccc21 ; Cn1c(=O)oc2cc(Nc3ccc(N4CCC(O)(C(F)(F)F)CC4)cc3)ccc21] 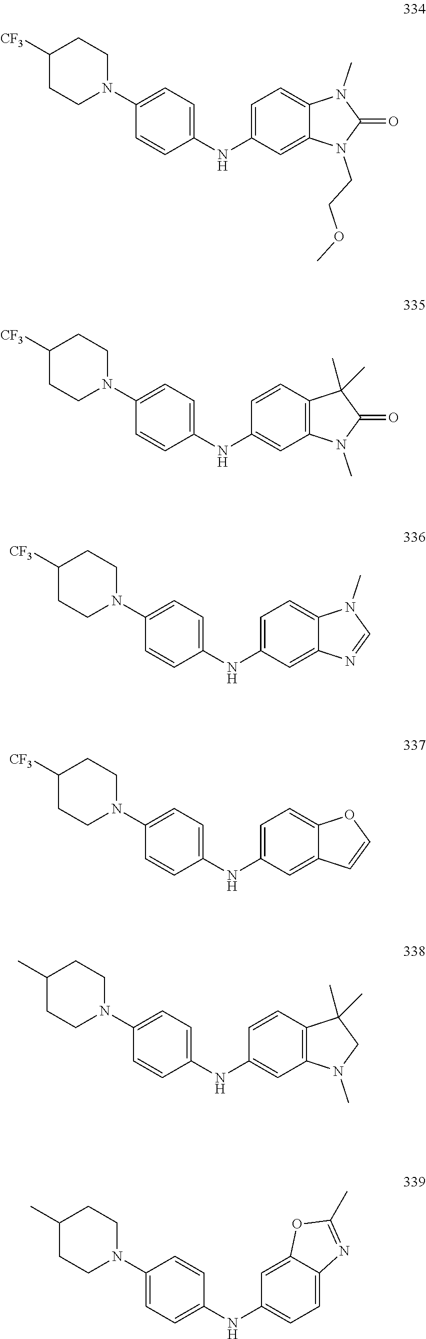 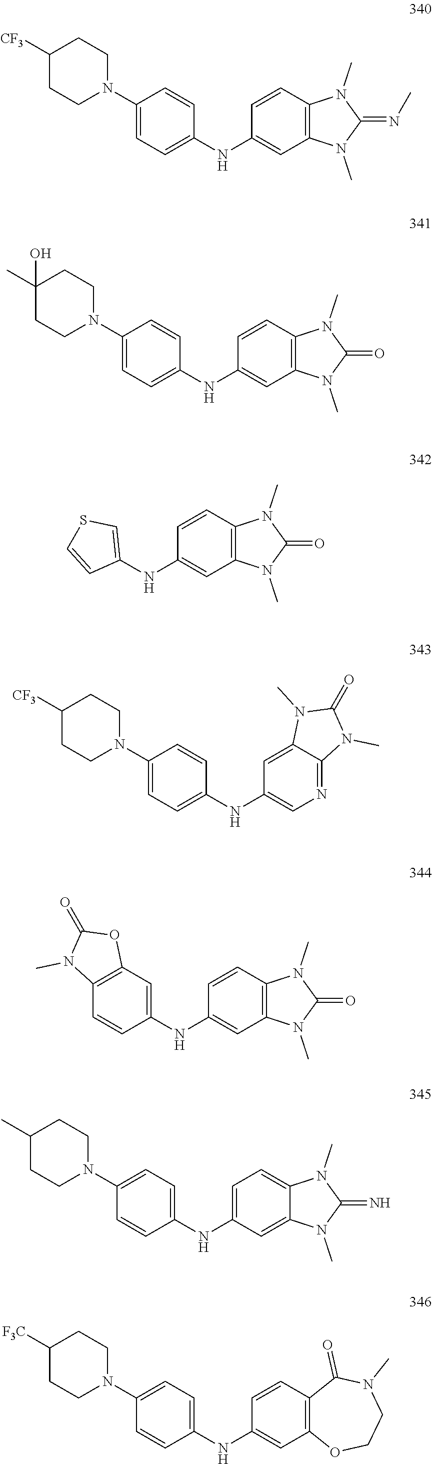 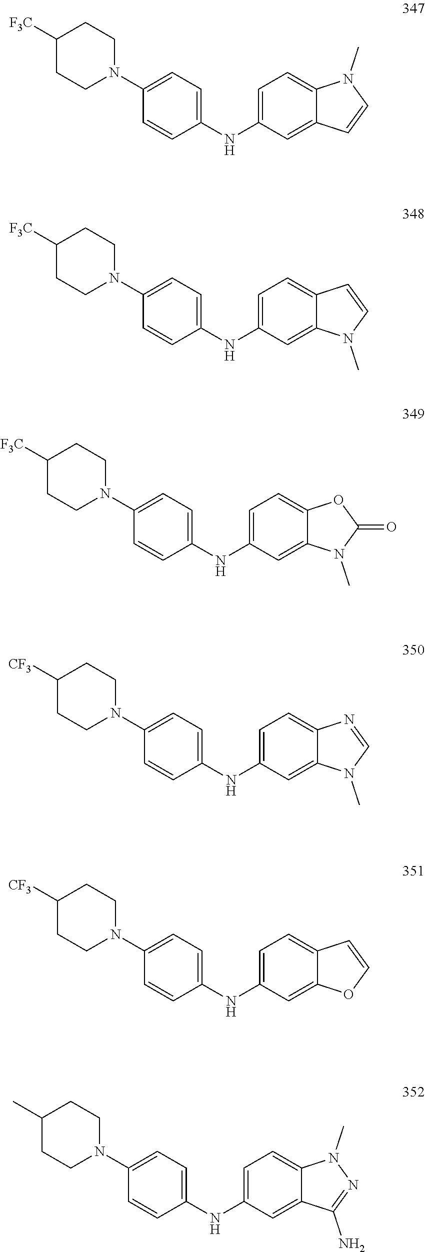 [SMILES: Cn1ccc2cc(Nc3ccc(N4CCC(C(F)(F)F)CC4)cc3)ccc21 ; Cn1ccc2ccc(Nc3ccc(N4CCC(C(F)(F)F)CC4)cc3)cc21 ; Cn1c(=O)oc2ccc(Nc3ccc(N4CCC(C(F)(F)F)CC4)cc3)cc21 ; Cn1cnc2ccc(Nc3ccc(N4CCC(C(F)(F)F)CC4)cc3)cc21 ; FC(F)(F)C1CCN(c2ccc(Nc3ccc4ccoc4c3)cc2)CC1 ; CC1CCN(c2ccc(Nc3ccc4c(c3)c(N)nn4C)cc2)CC1] 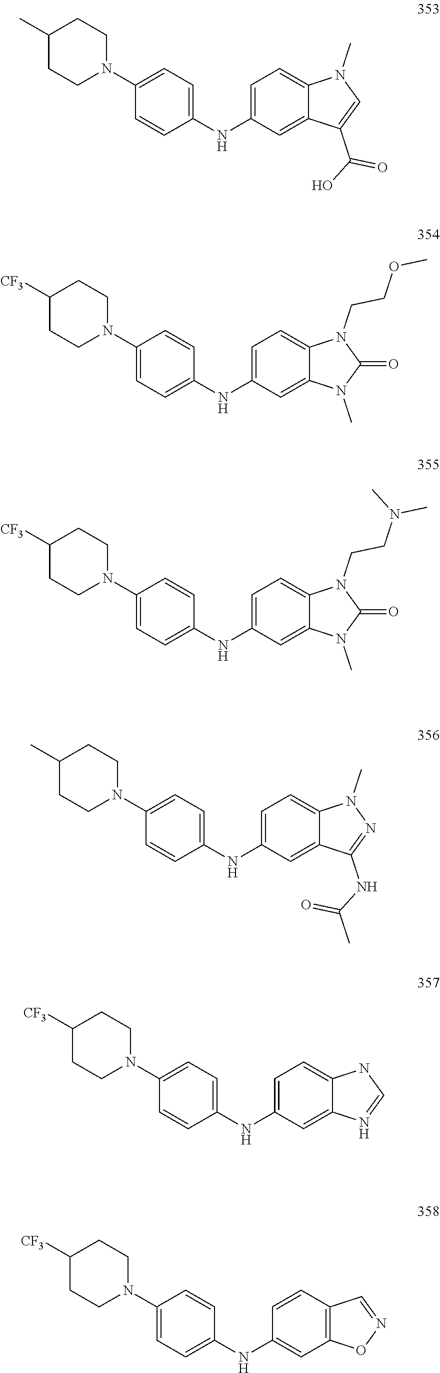 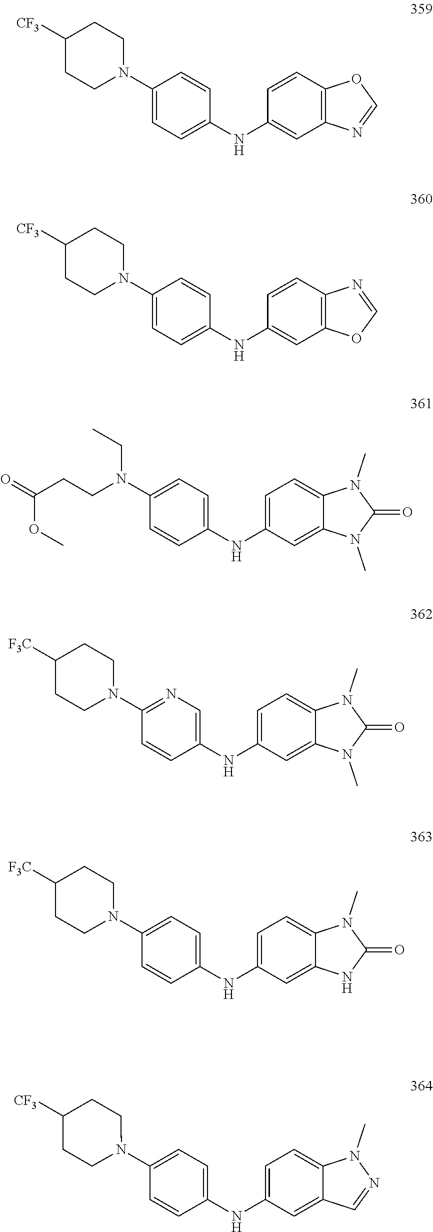 [SMILES: FC(F)(F)C1CCN(c2ccc(Nc3ccc4ocnc4c3)cc2)CC1 ; FC(F)(F)C1CCN(c2ccc(Nc3ccc4ncoc4c3)cc2)CC1 ; CCN(CCC(=O)OC)c1ccc(Nc2ccc3c(c2)n(C)c(=O)n3C)cc1 ; Cn1c(=O)n(C)c2cc(Nc3ccc(N4CCC(C(F)(F)F)CC4)nc3)ccc21 ; Cn1c(=O)[nH]c2cc(Nc3ccc(N4CCC(C(F)(F)F)CC4)cc3)ccc21 ; Cn1ncc2cc(Nc3ccc(N4CCC(C(F)(F)F)CC4)cc3)ccc21] 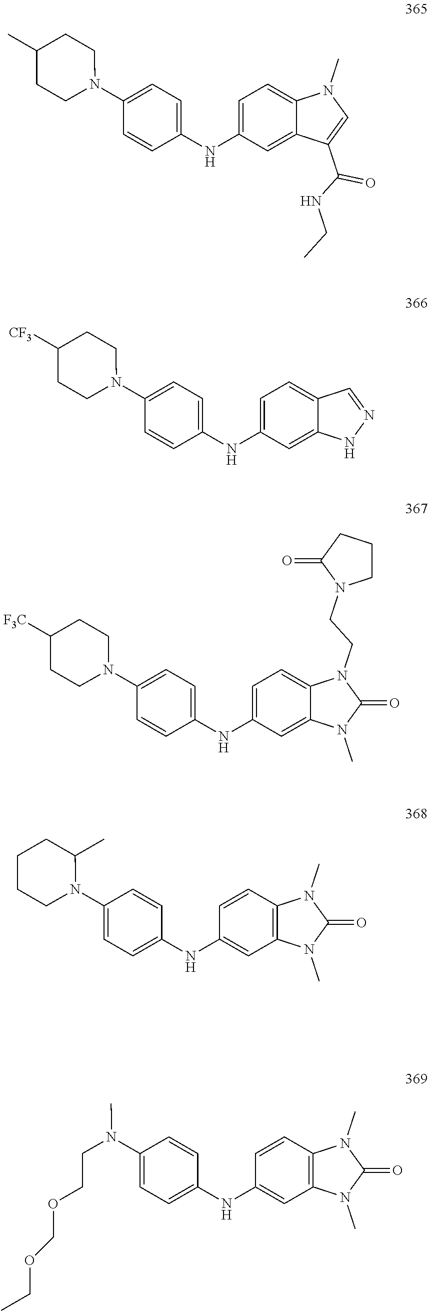 [SMILES: CCNC(=O)c1cn(C)c2ccc(Nc3ccc(N4CCC(C)CC4)cc3)cc12 ; FC(F)(F)C1CCN(c2ccc(Nc3ccc4cn[nH]c4c3)cc2)CC1 ; Cn1c(=O)n(CCN2CCCC2=O)c2ccc(Nc3ccc(N4CCC(C(F)(F)F)CC4)cc3)cc21 ; CC1CCCCN1c1ccc(Nc2ccc3c(c2)n(C)c(=O)n3C)cc1 ; CCOCOCCN(C)c1ccc(Nc2ccc3c(c2)n(C)c(=O)n3C)cc1] 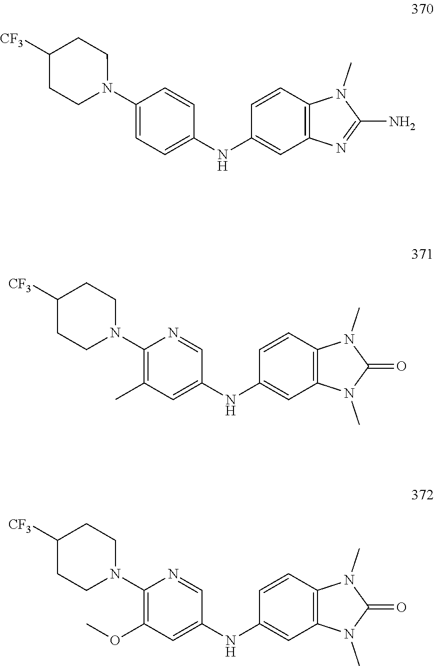 [SMILES: Cn1c(N)nc2cc(Nc3ccc(N4CCC(C(F)(F)F)CC4)cc3)ccc21 ; Cc1cc(Nc2ccc3c(c2)n(C)c(=O)n3C)cnc1N1CCC(C(F)(F)F)CC1 ; COc1cc(Nc2ccc3c(c2)n(C)c(=O)n3C)cnc1N1CCC(C(F)(F)F)CC1] 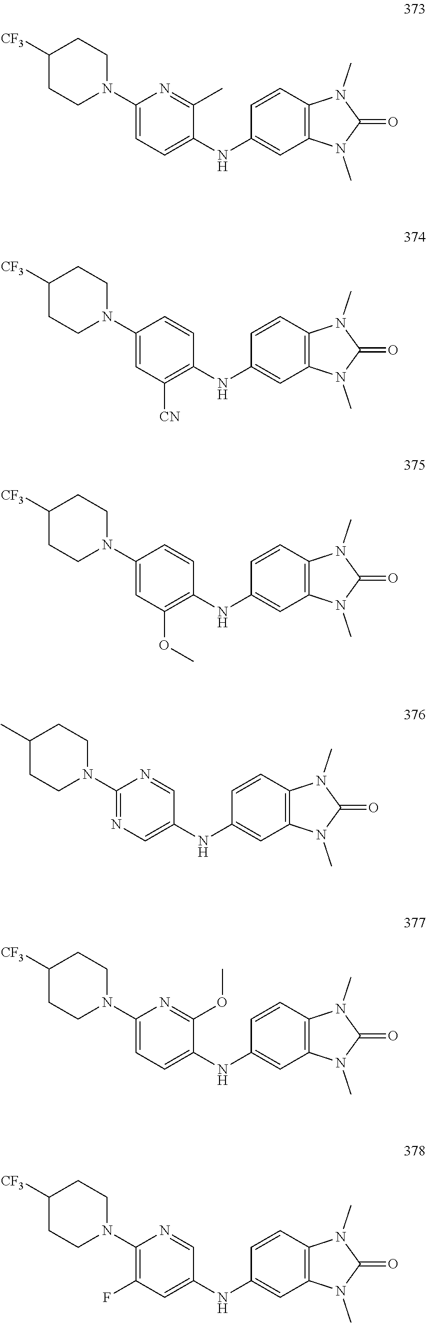 [SMILES: Cc1nc(N2CCC(C(F)(F)F)CC2)ccc1Nc1ccc2c(c1)n(C)c(=O)n2C ; Cn1c(=O)n(C)c2cc(Nc3ccc(N4CCC(C(F)(F)F)CC4)cc3C#N)ccc21 ; COc1cc(N2CCC(C(F)(F)F)CC2)ccc1Nc1ccc2c(c1)n(C)c(=O)n2C ; CC1CCN(c2ncc(Nc3ccc4c(c3)n(C)c(=O)n4C)cn2)CC1 ; COc1nc(N2CCC(C(F)(F)F)CC2)ccc1Nc1ccc2c(c1)n(C)c(=O)n2C ; Cn1c(=O)n(C)c2cc(Nc3cnc(N4CCC(C(F)(F)F)CC4)c(F)c3)ccc21] 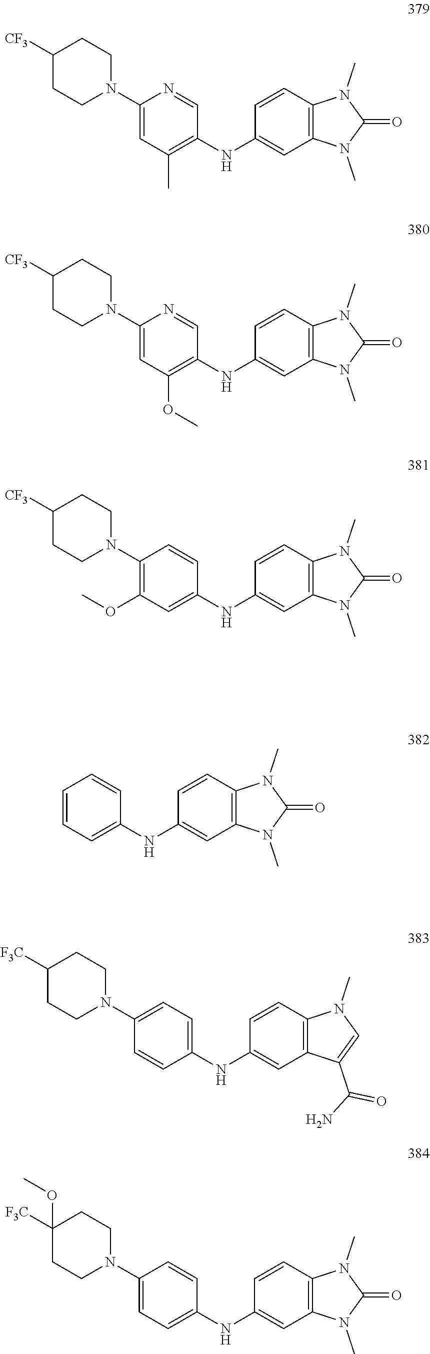 [SMILES: Cc1cc(N2CCC(C(F)(F)F)CC2)ncc1Nc1ccc2c(c1)n(C)c(=O)n2C ; COc1cc(N2CCC(C(F)(F)F)CC2)ncc1Nc1ccc2c(c1)n(C)c(=O)n2C ; COc1cc(Nc2ccc3c(c2)n(C)c(=O)n3C)ccc1N1CCC(C(F)(F)F)CC1 ; Cn1c(=O)n(C)c2cc(Nc3ccccc3)ccc21 ; Cn1cc(C(N)=O)c2cc(Nc3ccc(N4CCC(C(F)(F)F)CC4)cc3)ccc21 ; COC1(C(F)(F)F)CCN(c2ccc(Nc3ccc4c(c3)n(C)c(=O)n4C)cc2)CC1] 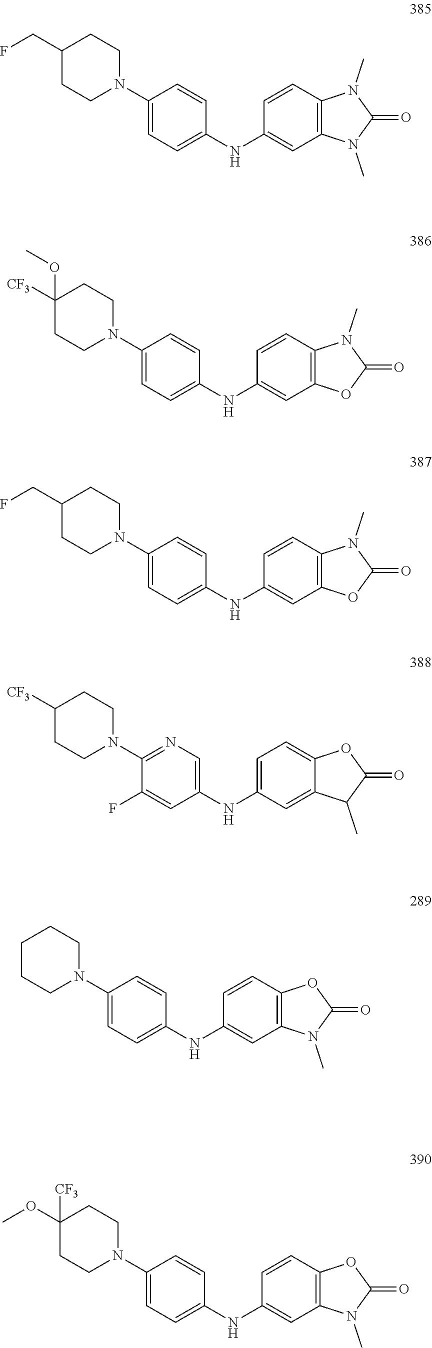 [SMILES: Cn1c(=O)n(C)c2cc(Nc3ccc(N4CCC(CF)CC4)cc3)ccc21 ; COC1(C(F)(F)F)CCN(c2ccc(Nc3ccc4c(c3)oc(=O)n4C)cc2)CC1 ; Cn1c(=O)oc2cc(Nc3ccc(N4CCC(CF)CC4)cc3)ccc21 ; CC1C(=O)Oc2ccc(Nc3cnc(N4CCC(C(F)(F)F)CC4)c(F)c3)cc21 ; Cn1c(=O)oc2ccc(Nc3ccc(N4CCCCC4)cc3)cc21 ; COC1(C(F)(F)F)CCN(c2ccc(Nc3ccc4oc(=O)n(C)c4c3)cc2)CC1] 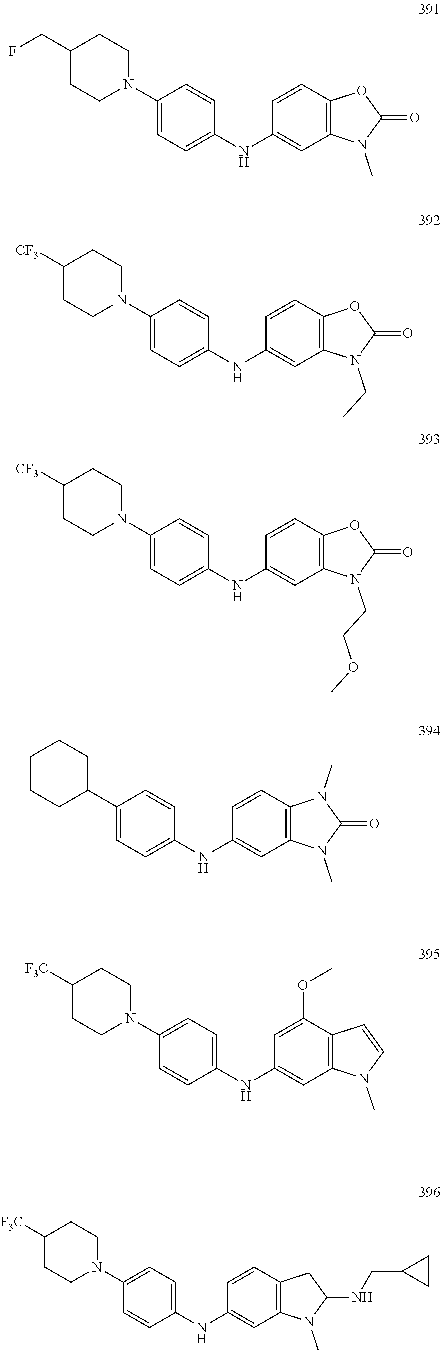 [SMILES: Cn1c(=O)oc2ccc(Nc3ccc(N4CCC(CF)CC4)cc3)cc21 ; CCn1c(=O)oc2ccc(Nc3ccc(N4CCC(C(F)(F)F)CC4)cc3)cc21 ; COCCn1c(=O)oc2ccc(Nc3ccc(N4CCC(C(F)(F)F)CC4)cc3)cc21 ; Cn1c(=O)n(C)c2cc(Nc3ccc(C4CCCCC4)cc3)ccc21 ; COc1cc(Nc2ccc(N3CCC(C(F)(F)F)CC3)cc2)cc2c1ccn2C ; CN1c2cc(Nc3ccc(N4CCC(C(F)(F)F)CC4)cc3)ccc2CC1NCC1CC1] 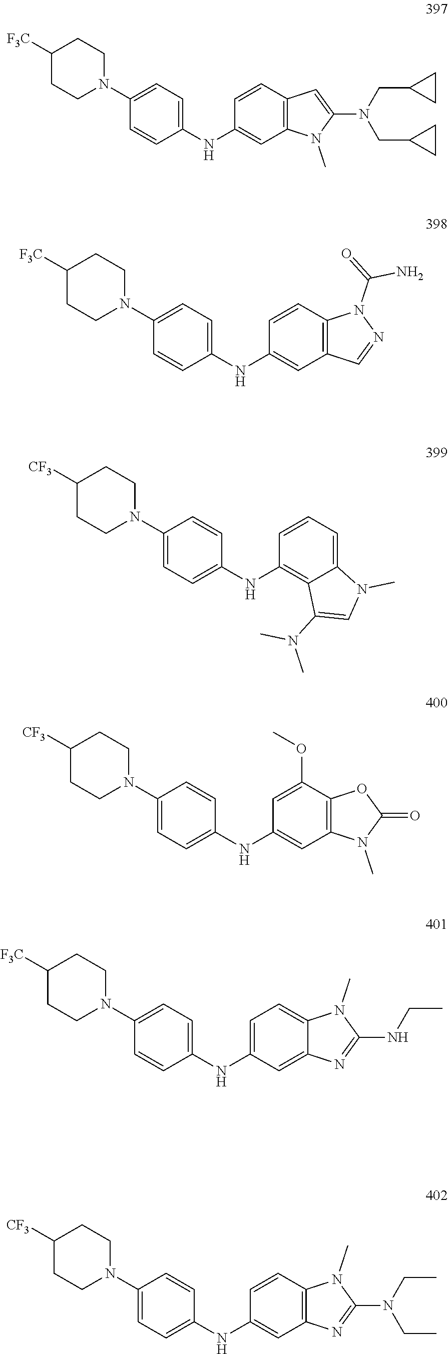 [SMILES: Cn1c(N(CC2CC2)CC2CC2)cc2ccc(Nc3ccc(N4CCC(C(F)(F)F)CC4)cc3)cc21 ; NC(=O)n1ncc2cc(Nc3ccc(N4CCC(C(F)(F)F)CC4)cc3)ccc21 ; CN(C)c1cn(C)c2cccc(Nc3ccc(N4CCC(C(F)(F)F)CC4)cc3)c12 ; COc1cc(Nc2ccc(N3CCC(C(F)(F)F)CC3)cc2)cc2c1oc(=O)n2C ; CCNc1nc2cc(Nc3ccc(N4CCC(C(F)(F)F)CC4)cc3)ccc2n1C ; CCN(CC)c1nc2cc(Nc3ccc(N4CCC(C(F)(F)F)CC4)cc3)ccc2n1C] 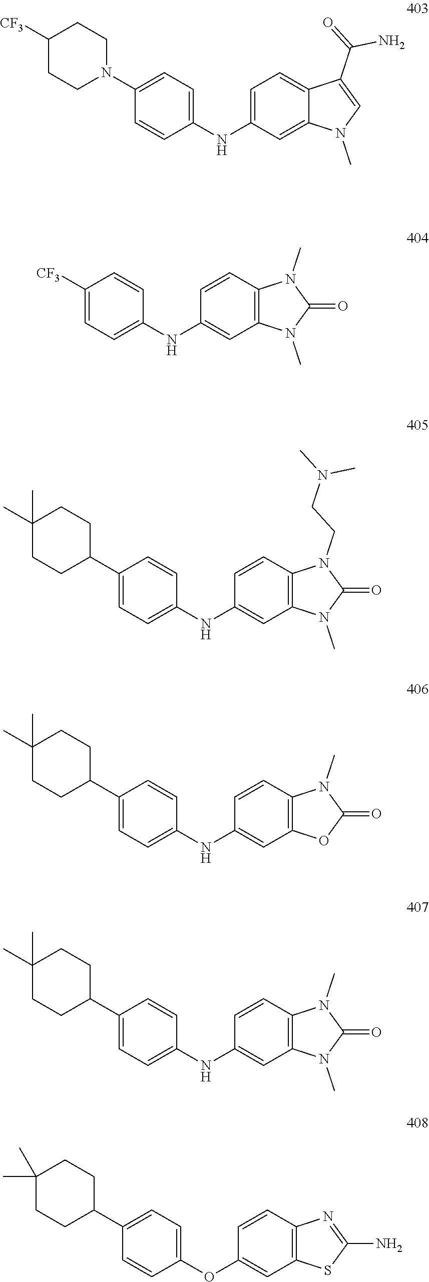 [SMILES: Cn1cc(C(N)=O)c2ccc(Nc3ccc(N4CCC(C(F)(F)F)CC4)cc3)cc21 ; Cn1c(=O)n(C)c2cc(Nc3ccc(C(F)(F)F)cc3)ccc21 ; CN(C)CCn1c(=O)n(C)c2cc(Nc3ccc(C4CCC(C)(C)CC4)cc3)ccc21 ; Cn1c(=O)oc2cc(Nc3ccc(C4CCC(C)(C)CC4)cc3)ccc21 ; Cn1c(=O)n(C)c2cc(Nc3ccc(C4CCC(C)(C)CC4)cc3)ccc21 ; CC1(C)CCC(c2ccc(Oc3ccc4nc(N)sc4c3)cc2)CC1] 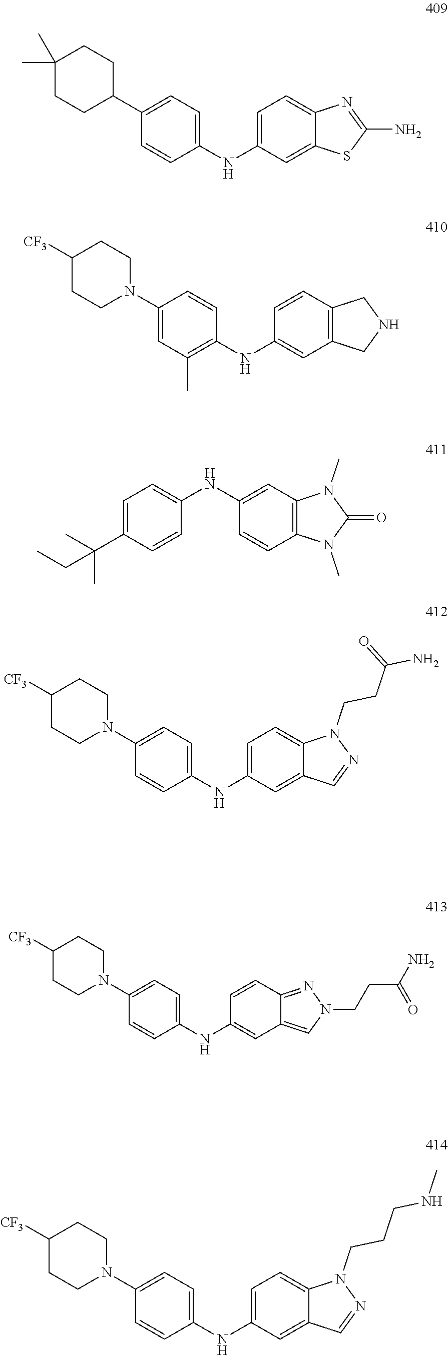 [SMILES: CC1(C)CCC(c2ccc(Nc3ccc4nc(N)sc4c3)cc2)CC1 ; Cc1cc(N2CCC(C(F)(F)F)CC2)ccc1Nc1ccc2c(c1)CNC2 ; CCC(C)(C)c1ccc(Nc2ccc3c(c2)n(C)c(=O)n3C)cc1 ; NC(=O)CCn1ncc2cc(Nc3ccc(N4CCC(C(F)(F)F)CC4)cc3)ccc21 ; NC(=O)CCn1cc2cc(Nc3ccc(N4CCC(C(F)(F)F)CC4)cc3)ccc2n1 ; CNCCCn1ncc2cc(Nc3ccc(N4CCC(C(F)(F)F)CC4)cc3)ccc21] 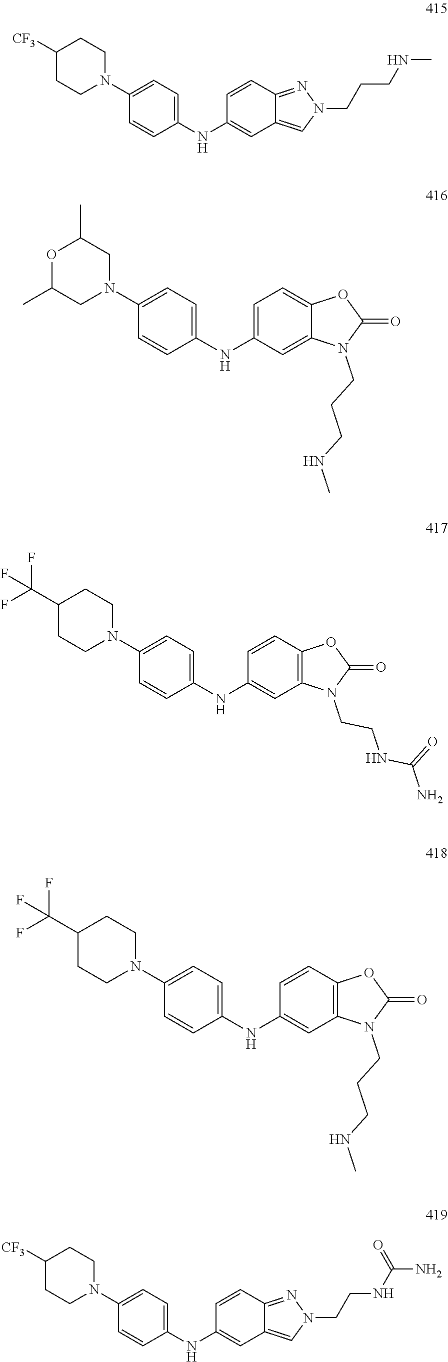 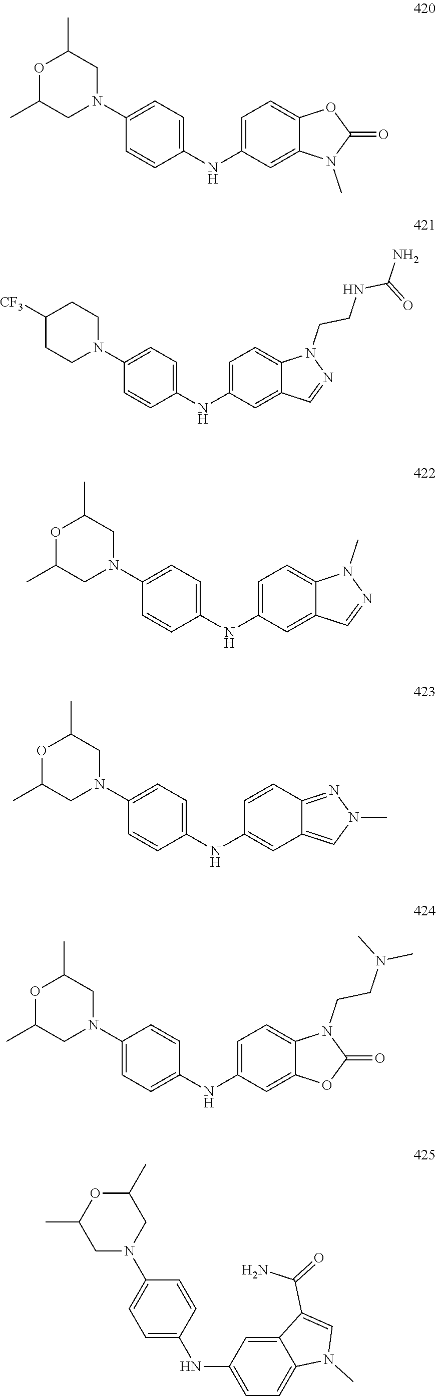 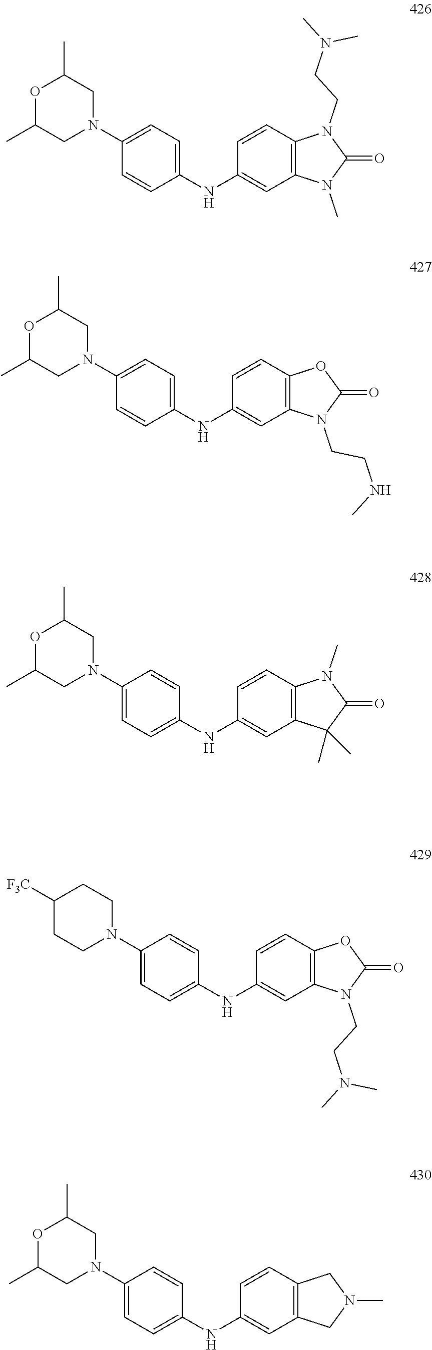 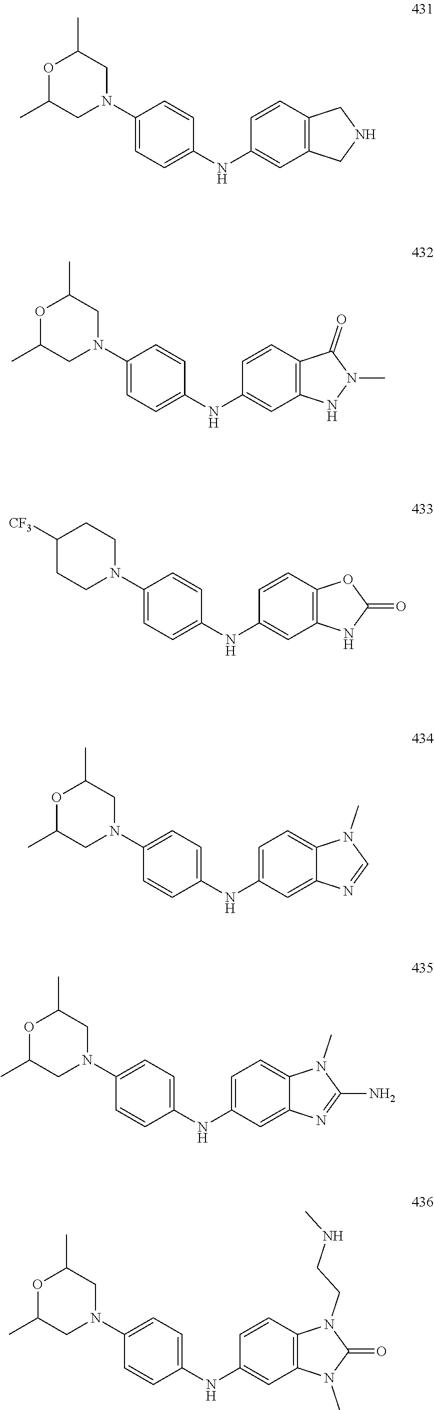 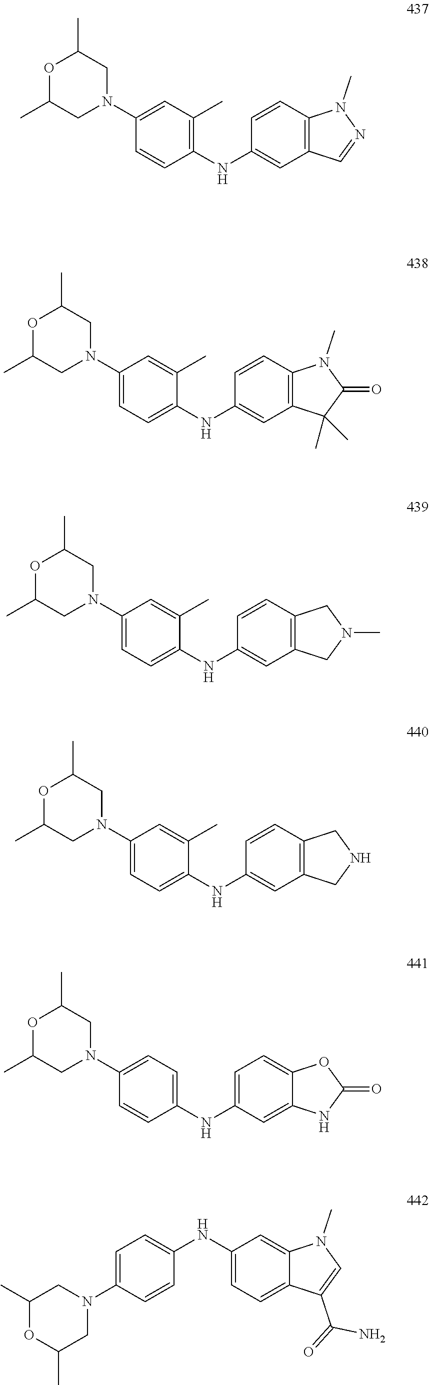 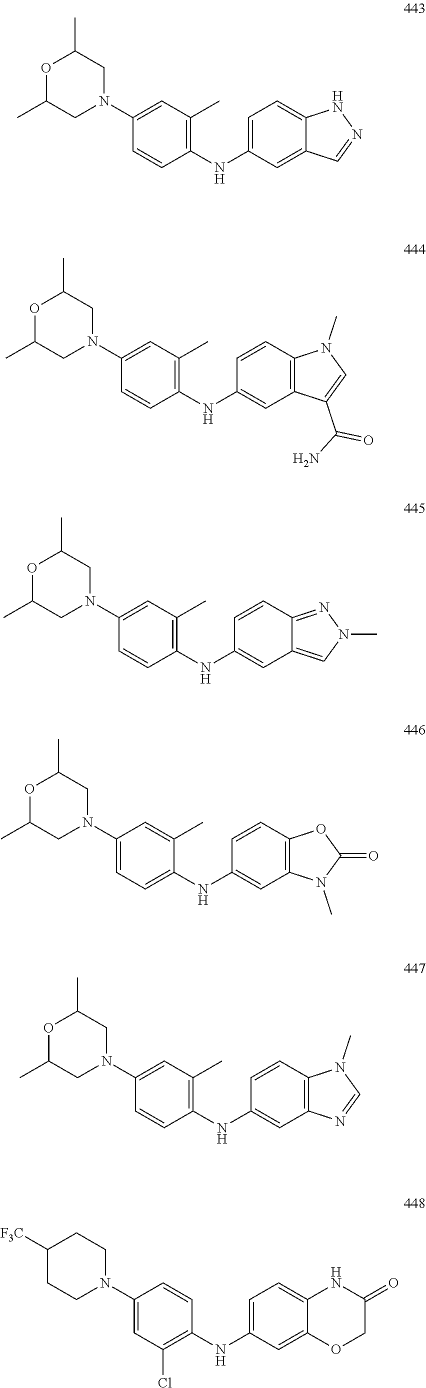 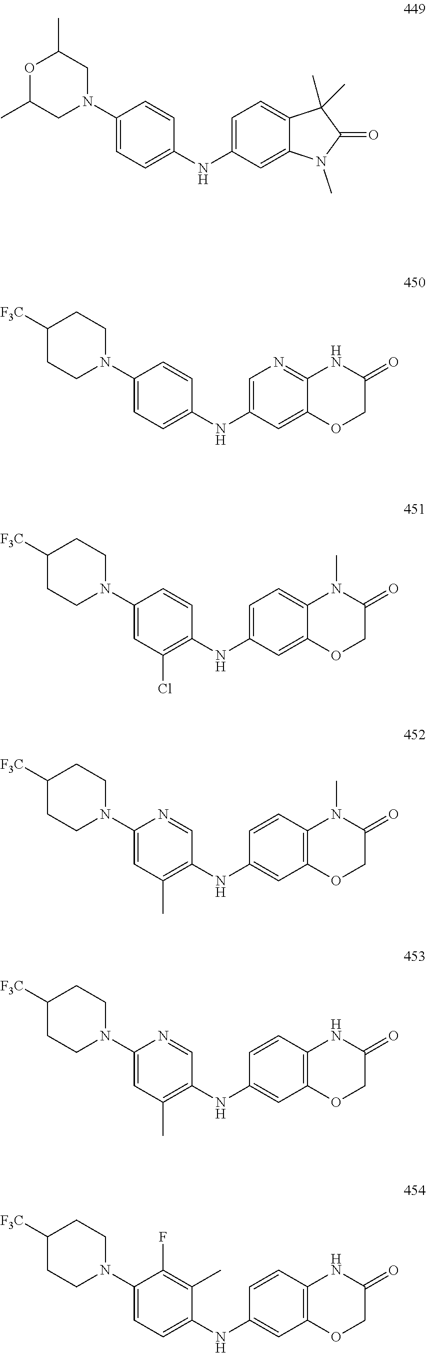 [SMILES: CC1CN(c2ccc(Nc3ccc4c(c3)N(C)C(=O)C4(C)C)cc2)CC(C)O1 ; O=C1COc2cc(Nc3ccc(N4CCC(C(F)(F)F)CC4)cc3)cnc2N1 ; CN1C(=O)COc2cc(Nc3ccc(N4CCC(C(F)(F)F)CC4)cc3Cl)ccc21 ; Cc1cc(N2CCC(C(F)(F)F)CC2)ncc1Nc1ccc2c(c1)OCC(=O)N2C ; Cc1cc(N2CCC(C(F)(F)F)CC2)ncc1Nc1ccc2c(c1)OCC(=O)N2 ; Cc1c(Nc2ccc3c(c2)OCC(=O)N3)ccc(N2CCC(C(F)(F)F)CC2)c1F] 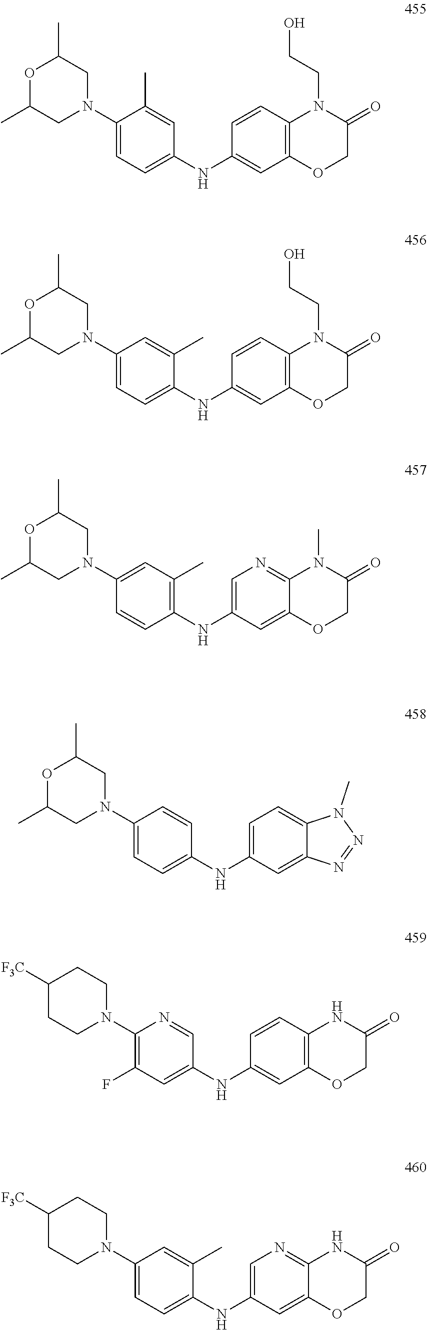 [SMILES: Cc1cc(Nc2ccc3c(c2)OCC(=O)N3CCO)ccc1N1CC(C)OC(C)C1 ; Cc1cc(N2CC(C)OC(C)C2)ccc1Nc1ccc2c(c1)OCC(=O)N2CCO ; Cc1cc(N2CC(C)OC(C)C2)ccc1Nc1cnc2c(c1)OCC(=O)N2C ; CC1CN(c2ccc(Nc3ccc4c(c3)nnn4C)cc2)CC(C)O1 ; O=C1COc2cc(Nc3cnc(N4CCC(C(F)(F)F)CC4)c(F)c3)ccc2N1 ; Cc1cc(N2CCC(C(F)(F)F)CC2)ccc1Nc1cnc2c(c1)OCC(=O)N2] 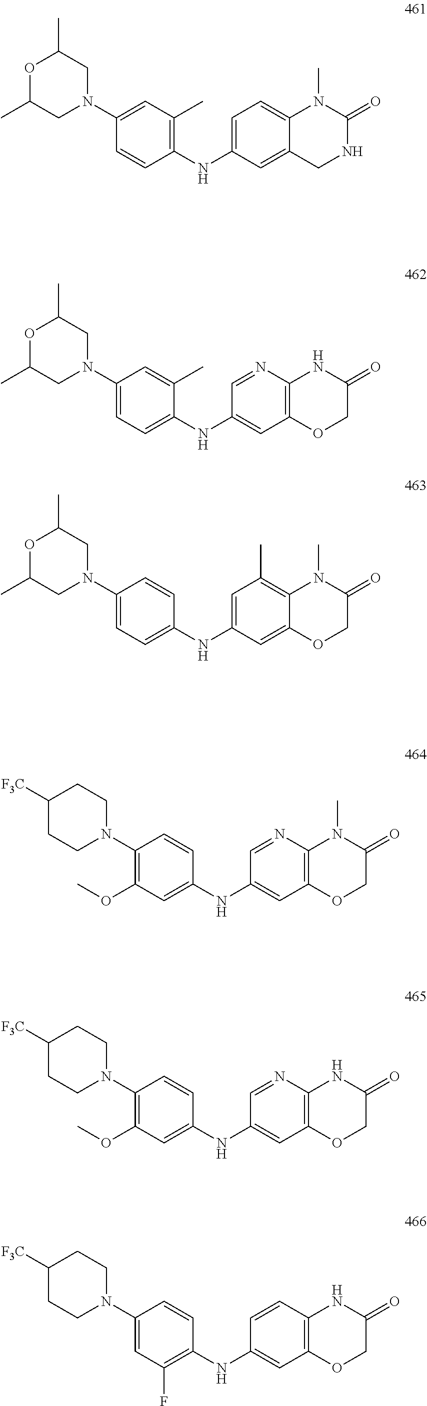 [SMILES: Cc1cc(N2CC(C)OC(C)C2)ccc1Nc1ccc2c(c1)CNC(=O)N2C ; Cc1cc(N2CC(C)OC(C)C2)ccc1Nc1cnc2c(c1)OCC(=O)N2 ; Cc1cc(Nc2ccc(N3CC(C)OC(C)C3)cc2)cc2c1N(C)C(=O)CO2 ; COc1cc(Nc2cnc3c(c2)OCC(=O)N3C)ccc1N1CCC(C(F)(F)F)CC1 ; COc1cc(Nc2cnc3c(c2)OCC(=O)N3)ccc1N1CCC(C(F)(F)F)CC1 ; O=C1COc2cc(Nc3ccc(N4CCC(C(F)(F)F)CC4)cc3F)ccc2N1] 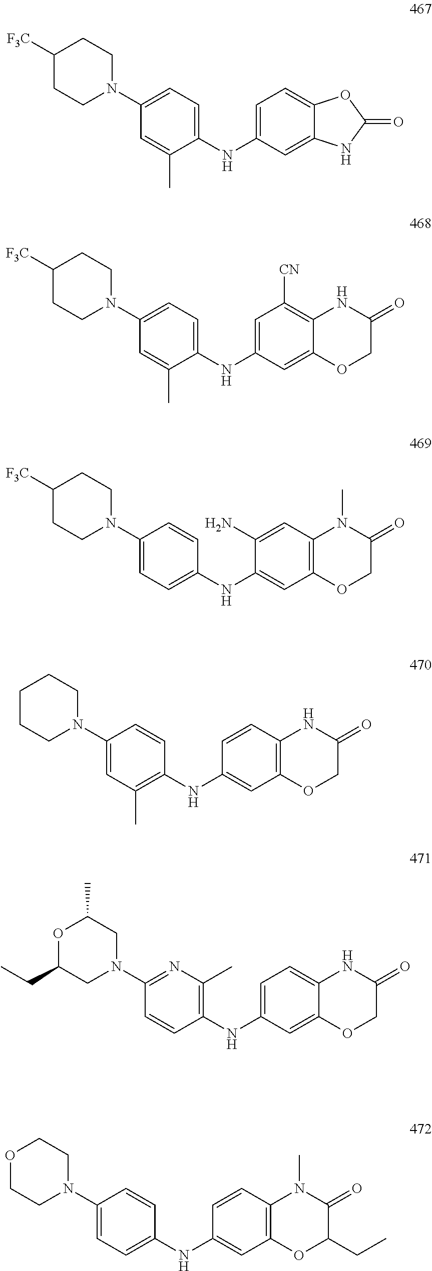 [SMILES: Cc1cc(N2CCC(C(F)(F)F)CC2)ccc1Nc1ccc2oc(=O)[nH]c2c1 ; Cc1cc(N2CCC(C(F)(F)F)CC2)ccc1Nc1cc(C#N)c2c(c1)OCC(=O)N2 ; CN1C(=O)COc2cc(Nc3ccc(N4CCC(C(F)(F)F)CC4)cc3)c(N)cc21 ; Cc1cc(N2CCCCC2)ccc1Nc1ccc2c(c1)OCC(=O)N2 ; CC[C@@H]1CN(c2ccc(Nc3ccc4c(c3)OCC(=O)N4)c(C)n2)C[C@@H](C)O1 ; CCC1Oc2cc(Nc3ccc(N4CCOCC4)cc3)ccc2N(C)C1=O] 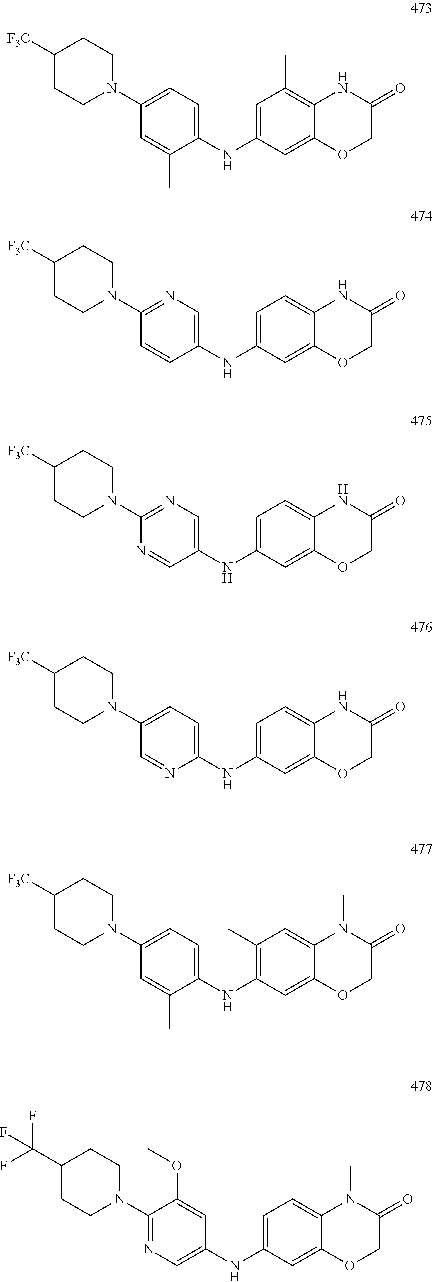 [SMILES: Cc1cc(N2CCC(C(F)(F)F)CC2)ccc1Nc1cc(C)c2c(c1)OCC(=O)N2 ; O=C1COc2cc(Nc3ccc(N4CCC(C(F)(F)F)CC4)nc3)ccc2N1 ; O=C1COc2cc(Nc3cnc(N4CCC(C(F)(F)F)CC4)nc3)ccc2N1 ; O=C1COc2cc(Nc3ccc(N4CCC(C(F)(F)F)CC4)cn3)ccc2N1 ; Cc1cc(N2CCC(C(F)(F)F)CC2)ccc1Nc1cc2c(cc1C)N(C)C(=O)CO2 ; COc1cc(Nc2ccc3c(c2)OCC(=O)N3C)cnc1N1CCC(C(F)(F)F)CC1] 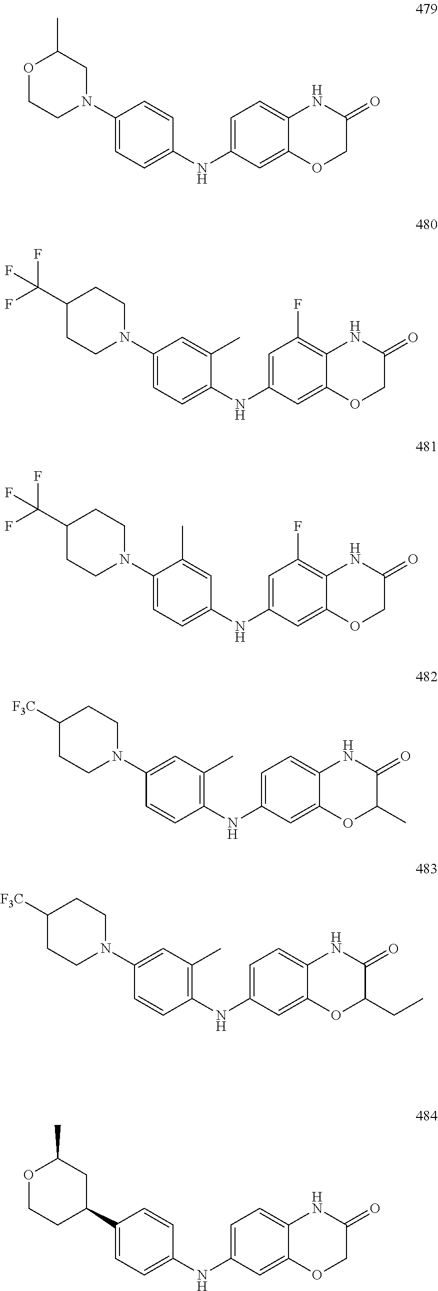 [SMILES: CC1CN(c2ccc(Nc3ccc4c(c3)OCC(=O)N4)cc2)CCO1 ; Cc1cc(N2CCC(C(F)(F)F)CC2)ccc1Nc1cc(F)c2c(c1)OCC(=O)N2 ; Cc1cc(Nc2cc(F)c3c(c2)OCC(=O)N3)ccc1N1CCC(C(F)(F)F)CC1 ; Cc1cc(N2CCC(C(F)(F)F)CC2)ccc1Nc1ccc2c(c1)OC(C)C(=O)N2 ; CCC1Oc2cc(Nc3ccc(N4CCC(C(F)(F)F)CC4)cc3C)ccc2NC1=O ; C[C@H]1C[C@@H](c2ccc(Nc3ccc4c(c3)OCC(=O)N4)cc2)CCO1] 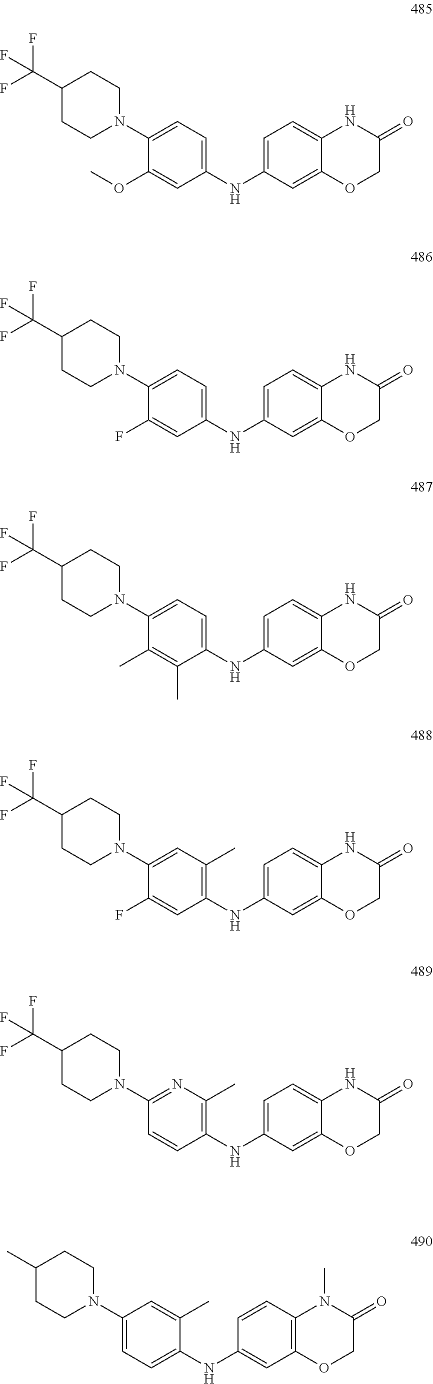 [SMILES: COc1cc(Nc2ccc3c(c2)OCC(=O)N3)ccc1N1CCC(C(F)(F)F)CC1 ; O=C1COc2cc(Nc3ccc(N4CCC(C(F)(F)F)CC4)c(F)c3)ccc2N1 ; Cc1c(Nc2ccc3c(c2)OCC(=O)N3)ccc(N2CCC(C(F)(F)F)CC2)c1C ; Cc1cc(N2CCC(C(F)(F)F)CC2)c(F)cc1Nc1ccc2c(c1)OCC(=O)N2 ; Cc1nc(N2CCC(C(F)(F)F)CC2)ccc1Nc1ccc2c(c1)OCC(=O)N2 ; Cc1cc(N2CCC(C)CC2)ccc1Nc1ccc2c(c1)OCC(=O)N2C] 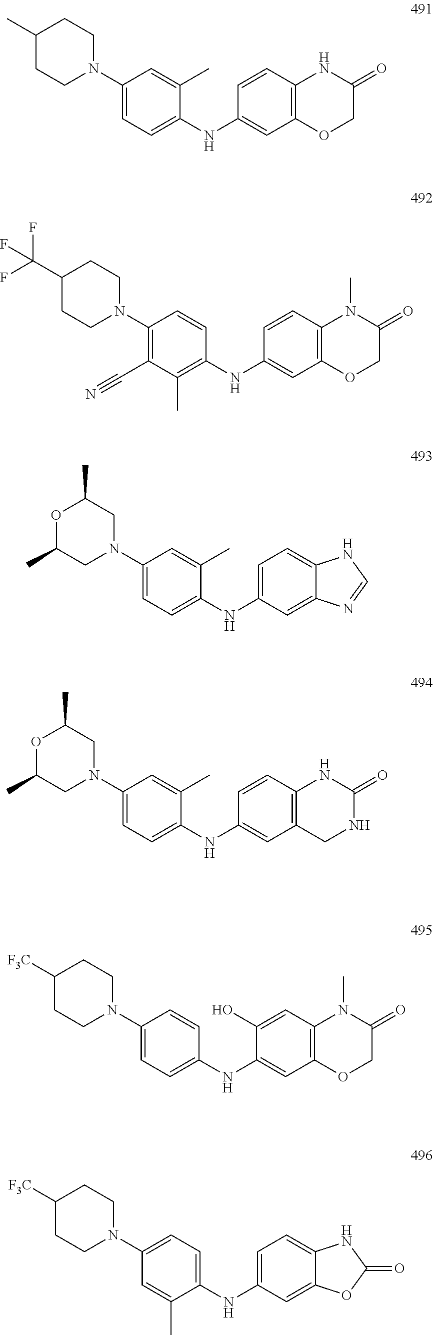 [SMILES: Cc1cc(N2CCC(C)CC2)ccc1Nc1ccc2c(c1)OCC(=O)N2 ; Cc1c(Nc2ccc3c(c2)OCC(=O)N3C)ccc(N2CCC(C(F)(F)F)CC2)c1C#N ; Cc1cc(N2C[C@@H](C)O[C@@H](C)C2)ccc1Nc1ccc2[nH]cnc2c1 ; Cc1cc(N2C[C@@H](C)O[C@@H](C)C2)ccc1Nc1ccc2c(c1)CNC(=O)N2 ; CN1C(=O)COc2cc(Nc3ccc(N4CCC(C(F)(F)F)CC4)cc3)c(O)cc21 ; Cc1cc(N2CCC(C(F)(F)F)CC2)ccc1Nc1ccc2[nH]c(=O)oc2c1] 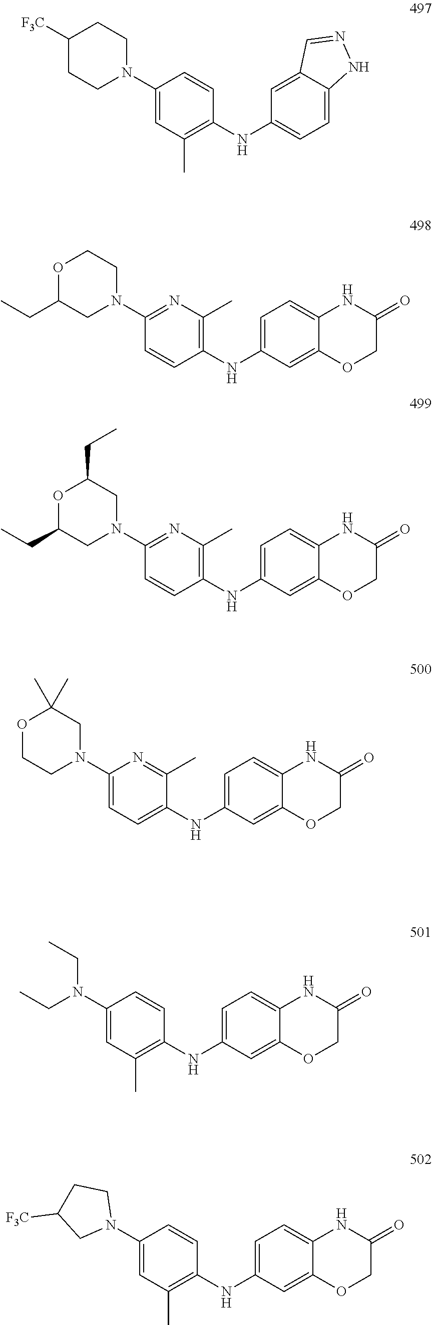 [SMILES: Cc1cc(N2CCC(C(F)(F)F)CC2)ccc1Nc1ccc2[nH]ncc2c1 ; CCC1CN(c2ccc(Nc3ccc4c(c3)OCC(=O)N4)c(C)n2)CCO1 ; CC[C@@H]1CN(c2ccc(Nc3ccc4c(c3)OCC(=O)N4)c(C)n2)C[C@H](CC)O1 ; Cc1nc(N2CCOC(C)(C)C2)ccc1Nc1ccc2c(c1)OCC(=O)N2 ; CCN(CC)c1ccc(Nc2ccc3c(c2)OCC(=O)N3)c(C)c1 ; Cc1cc(N2CCC(C(F)(F)F)C2)ccc1Nc1ccc2c(c1)OCC(=O)N2] 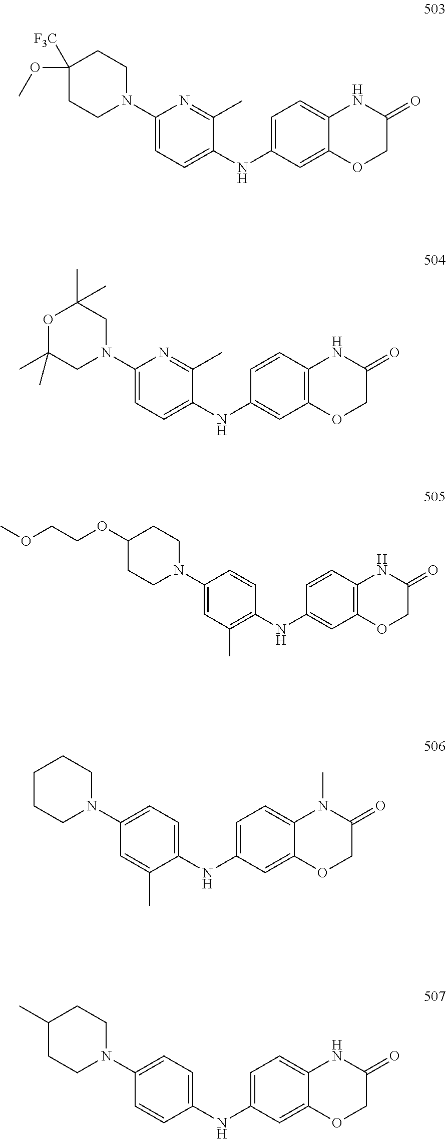 [SMILES: COC1(C(F)(F)F)CCN(c2ccc(Nc3ccc4c(c3)OCC(=O)N4)c(C)n2)CC1 ; Cc1nc(N2CC(C)(C)OC(C)(C)C2)ccc1Nc1ccc2c(c1)OCC(=O)N2 ; COCCOC1CCN(c2ccc(Nc3ccc4c(c3)OCC(=O)N4)c(C)c2)CC1 ; Cc1cc(N2CCCCC2)ccc1Nc1ccc2c(c1)OCC(=O)N2C ; CC1CCN(c2ccc(Nc3ccc4c(c3)OCC(=O)N4)cc2)CC1] 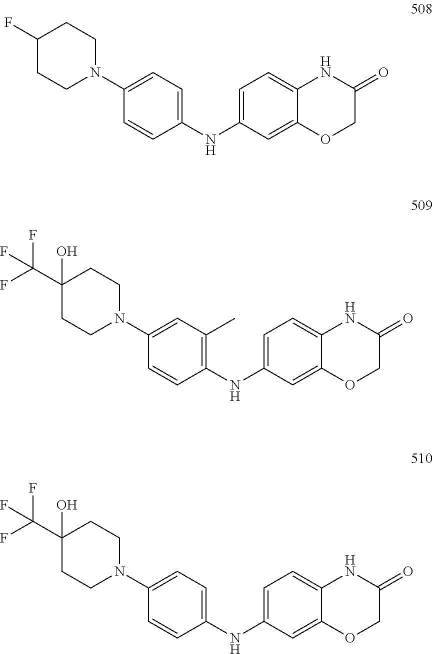 [SMILES: O=C1COc2cc(Nc3ccc(N4CCC(F)CC4)cc3)ccc2N1 ; Cc1cc(N2CCC(O)(C(F)(F)F)CC2)ccc1Nc1ccc2c(c1)OCC(=O)N2 ; O=C1COc2cc(Nc3ccc(N4CCC(O)(C(F)(F)F)CC4)cc3)ccc2N1] 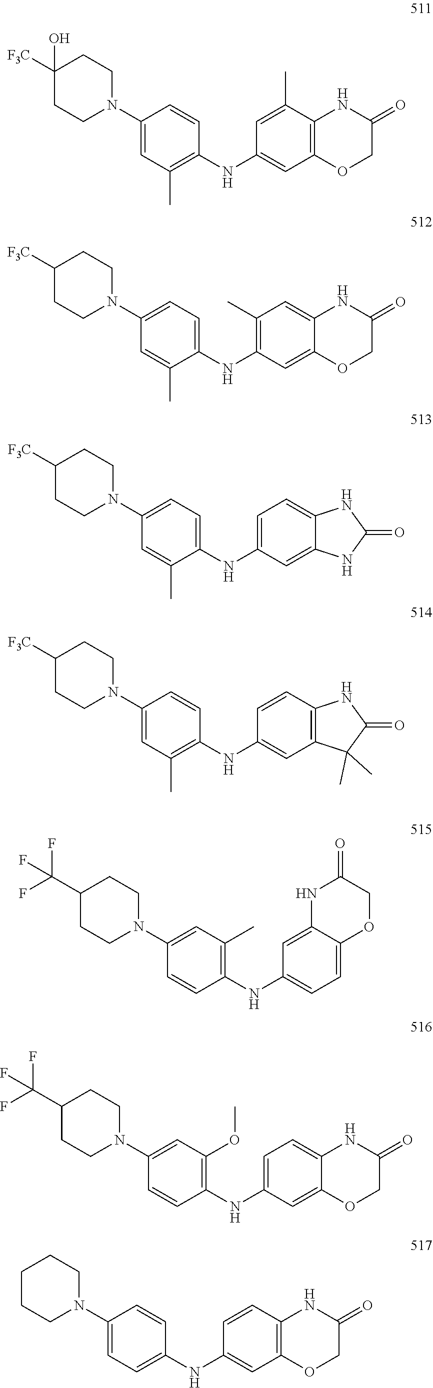 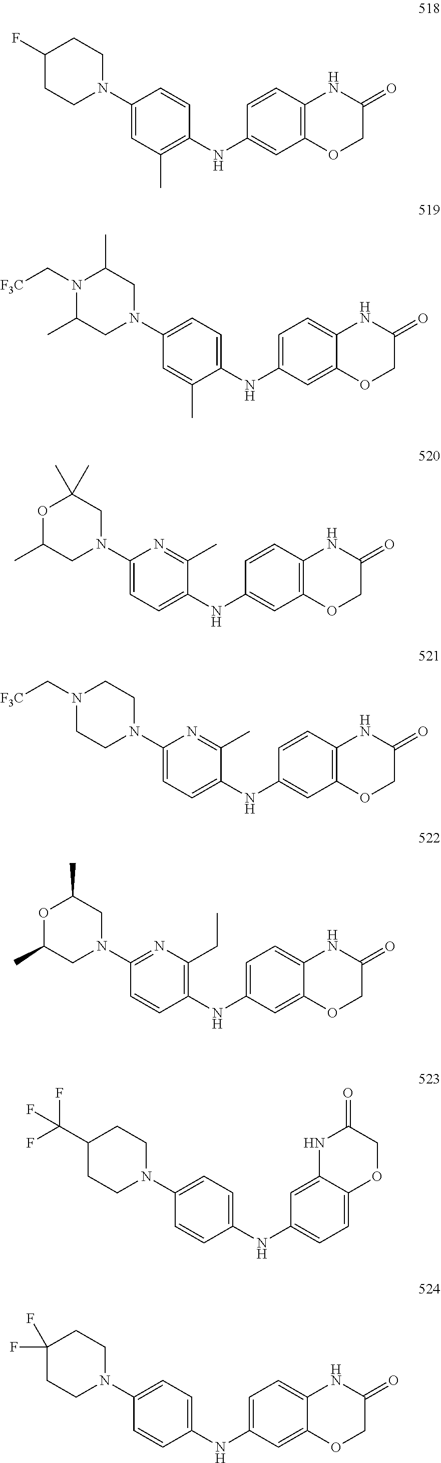 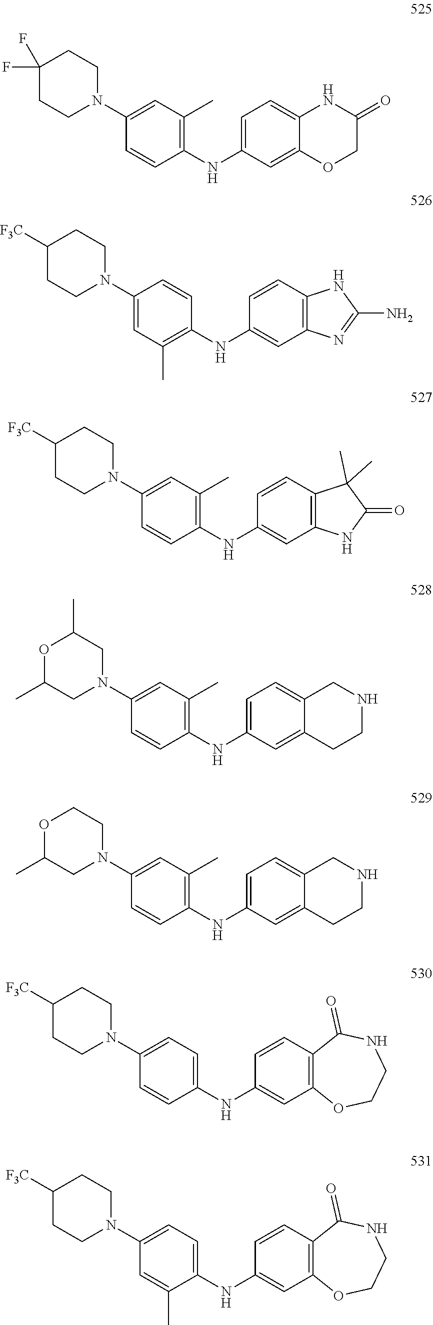 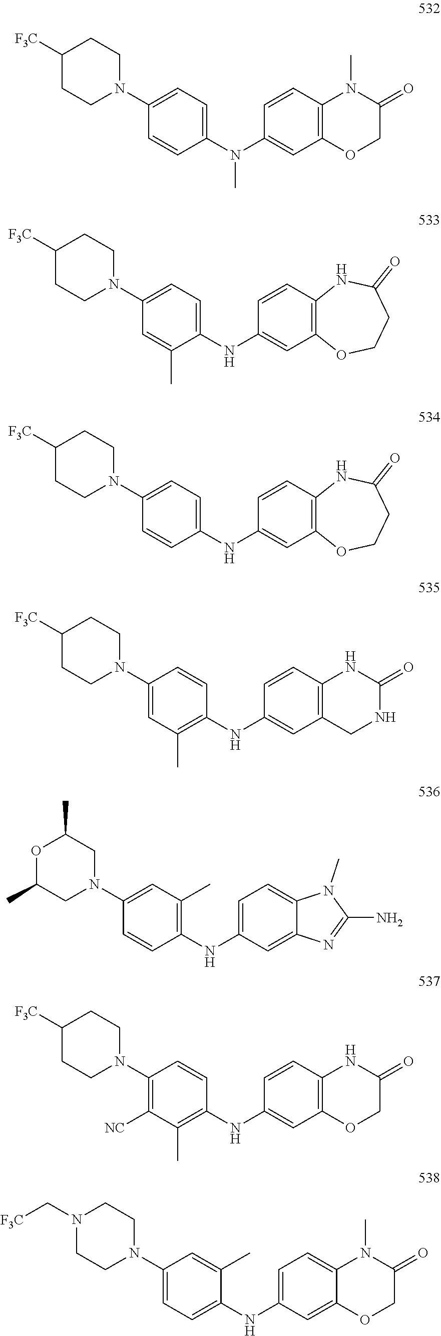 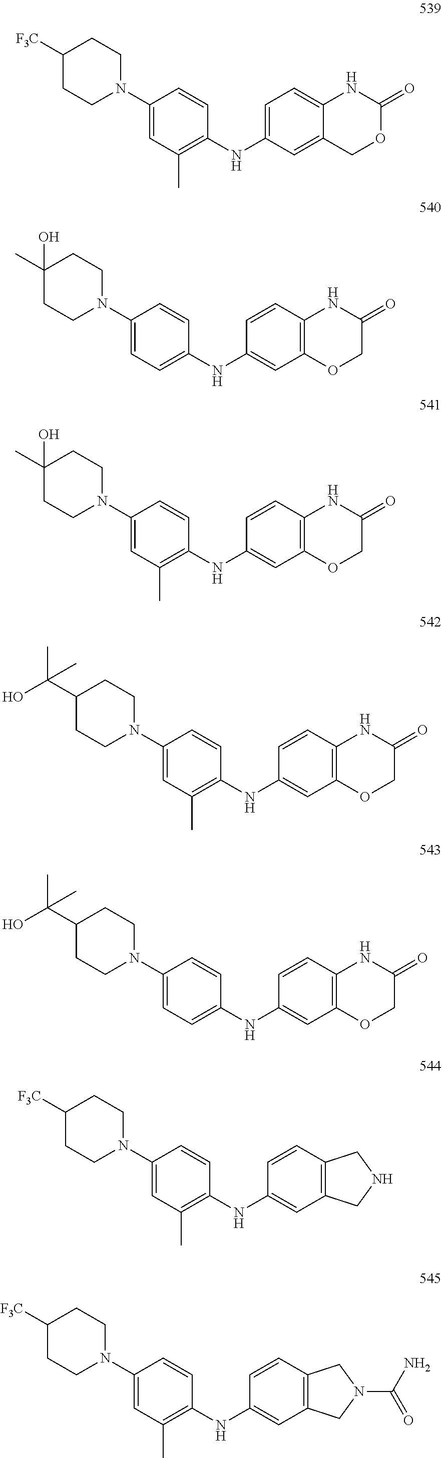 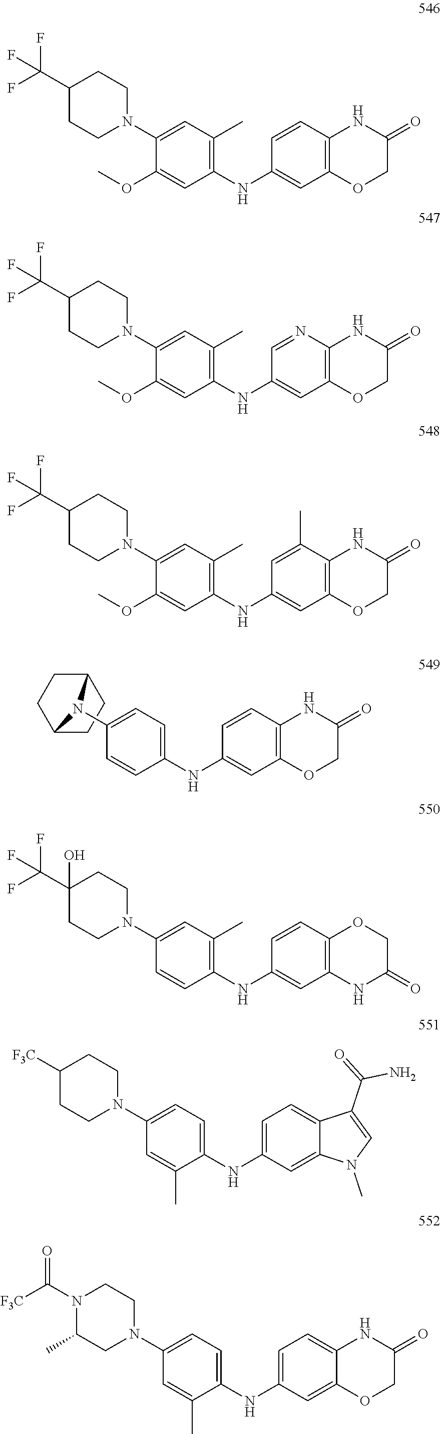 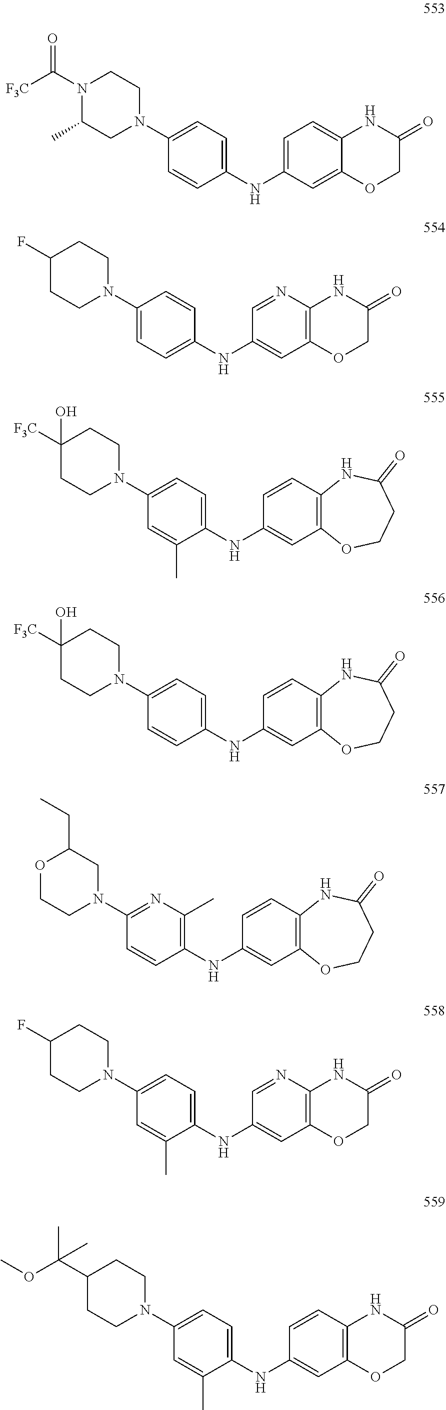 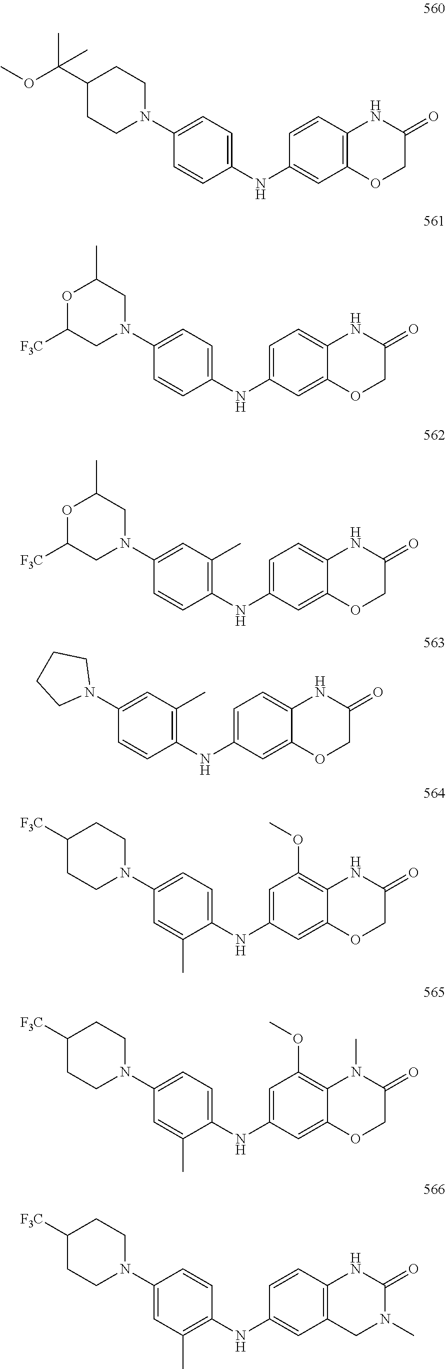 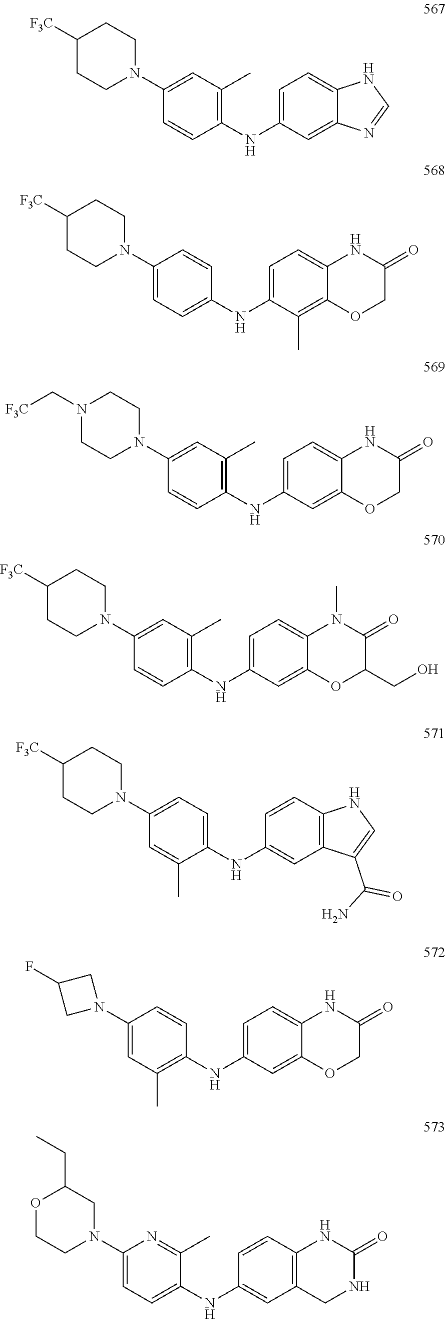 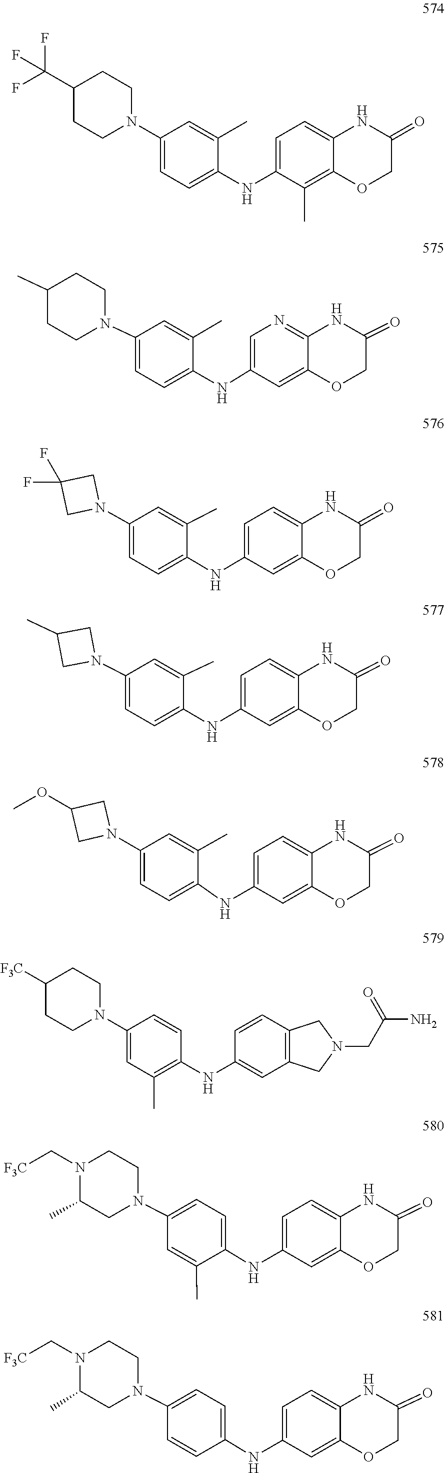 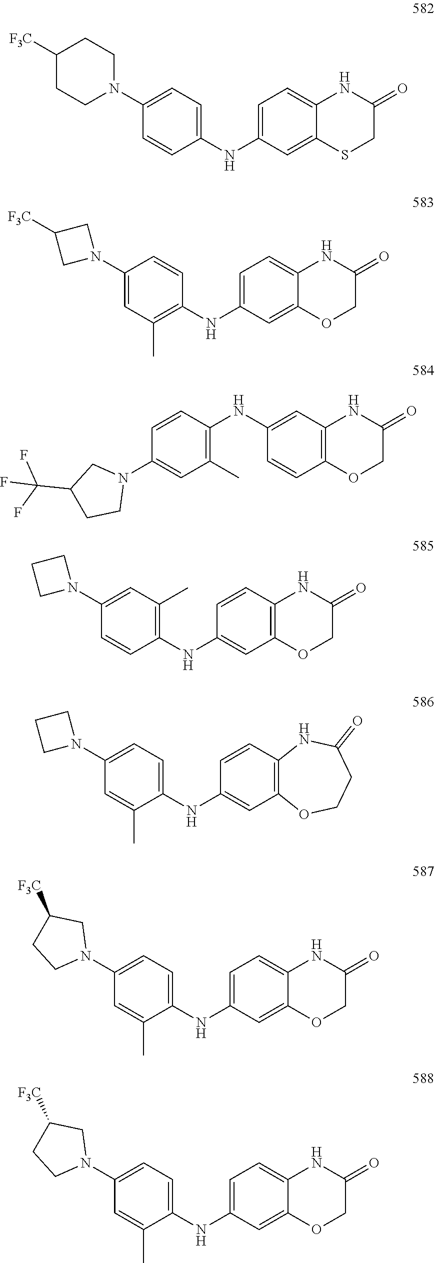 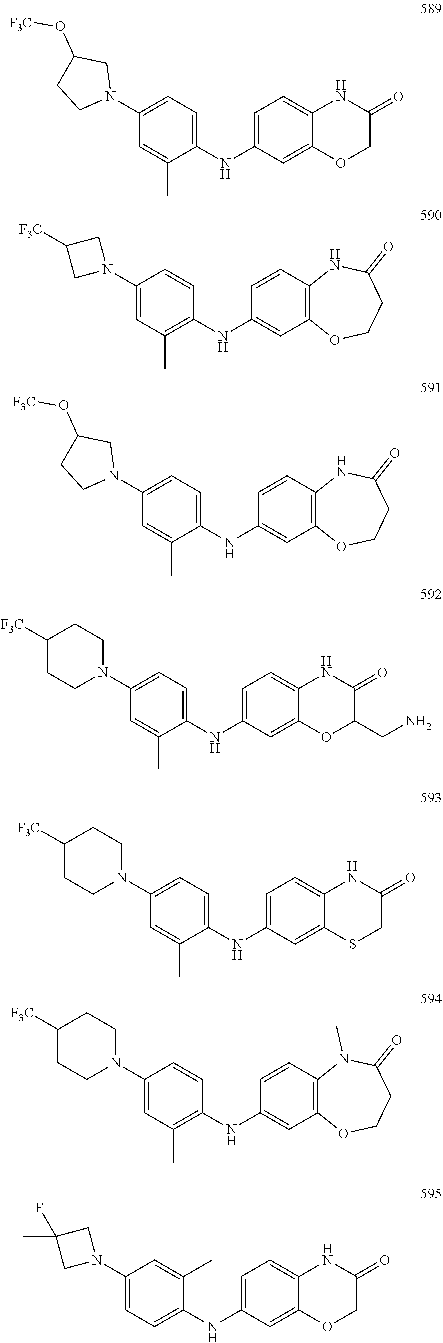 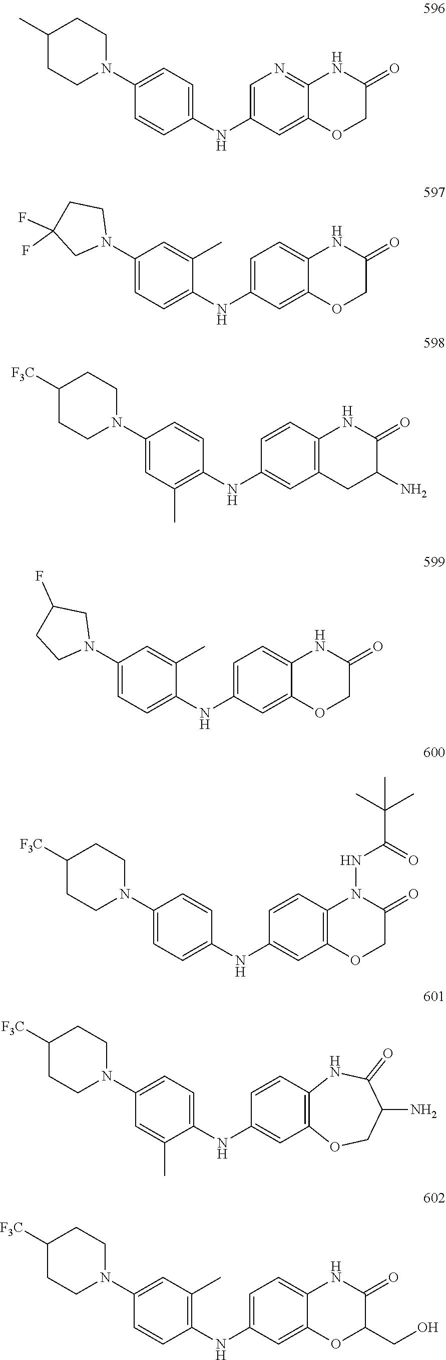 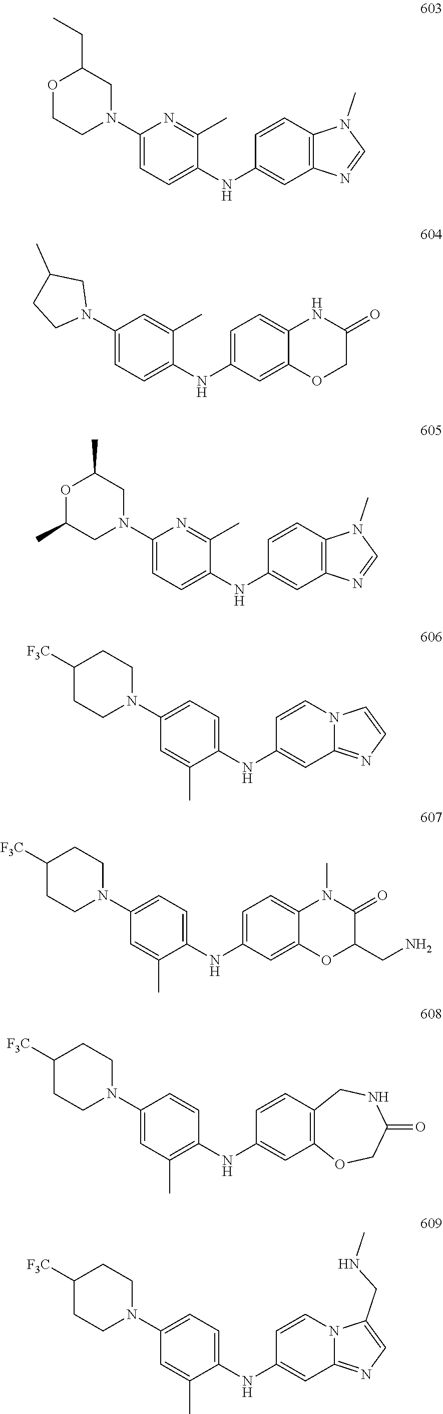 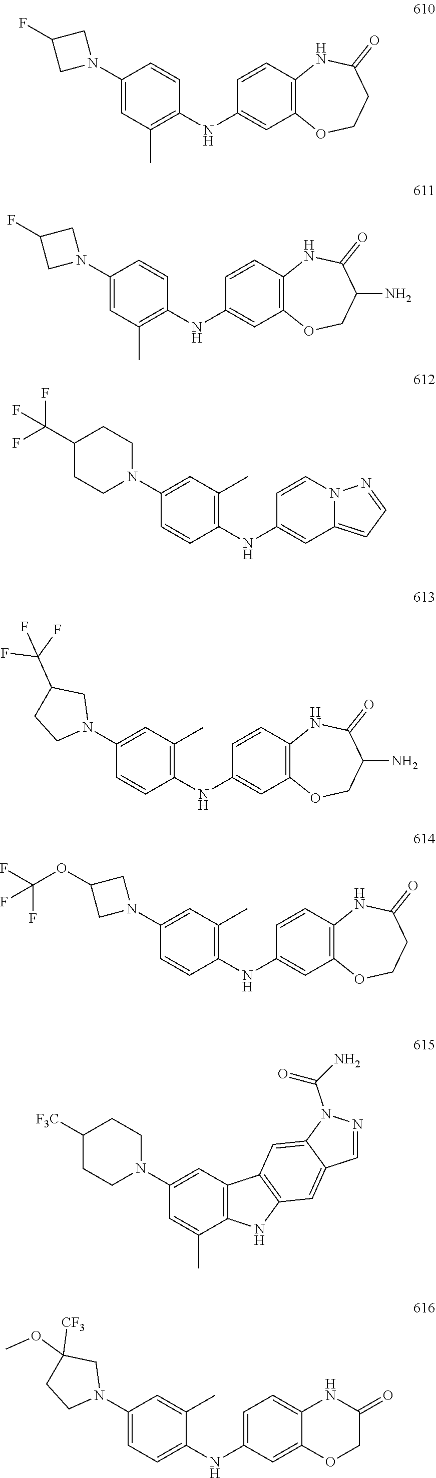 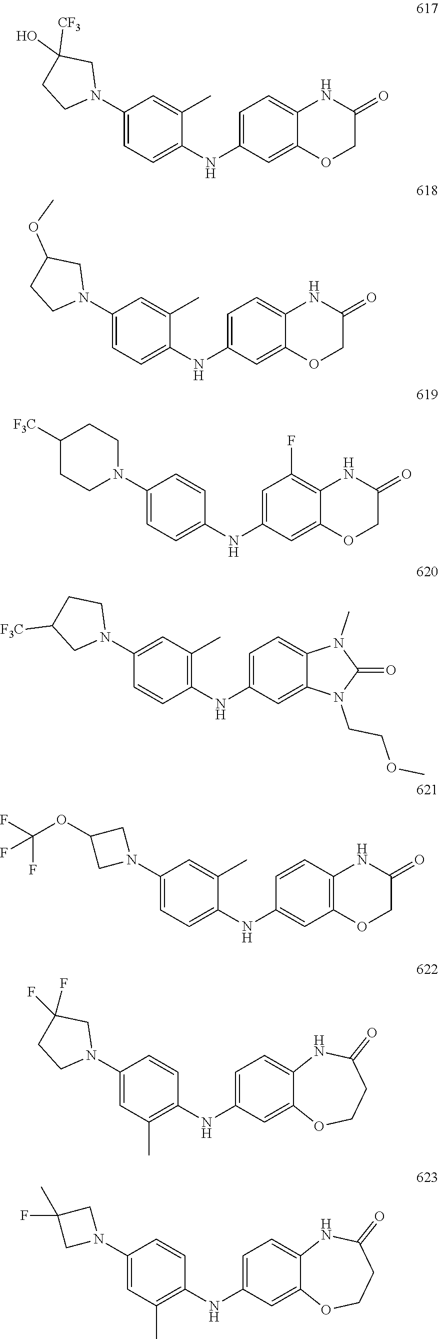 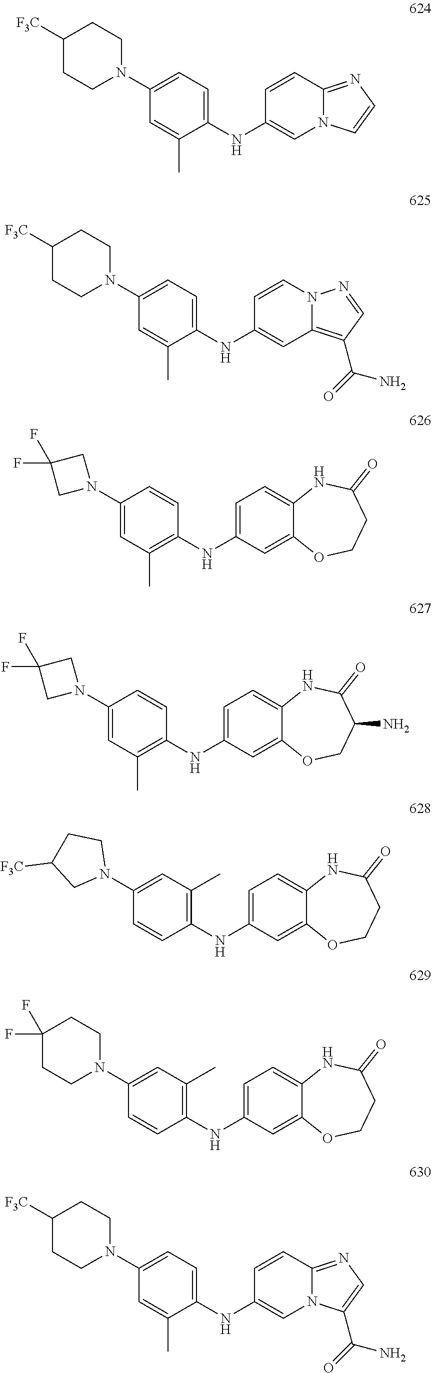 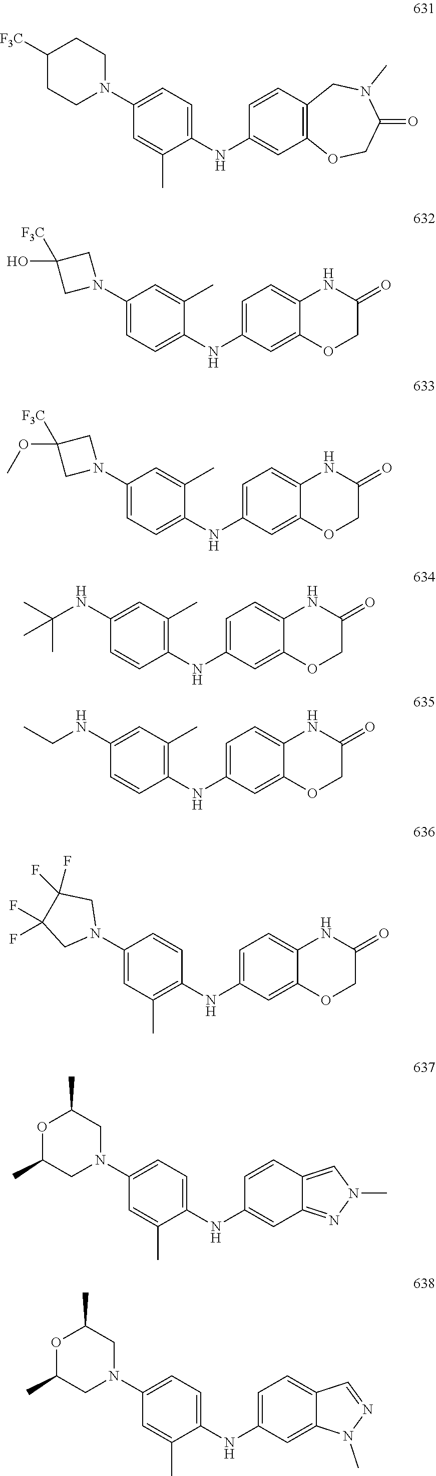 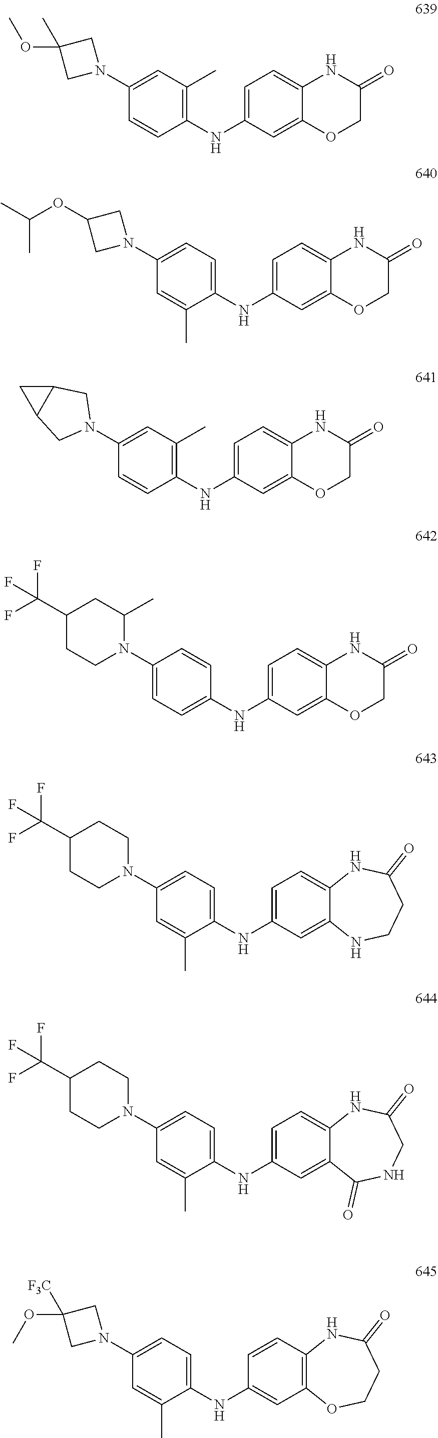 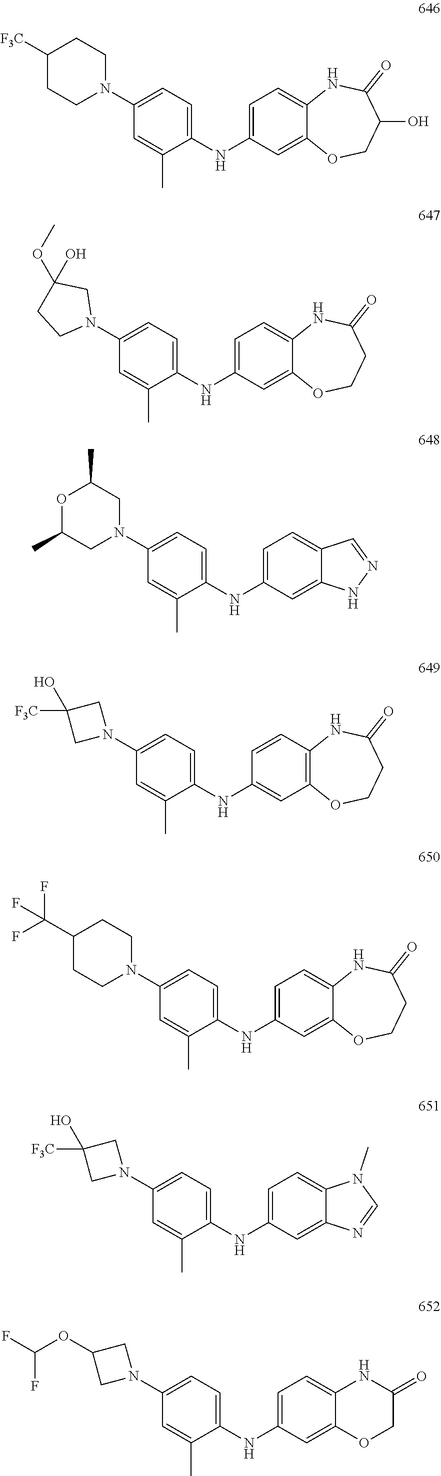 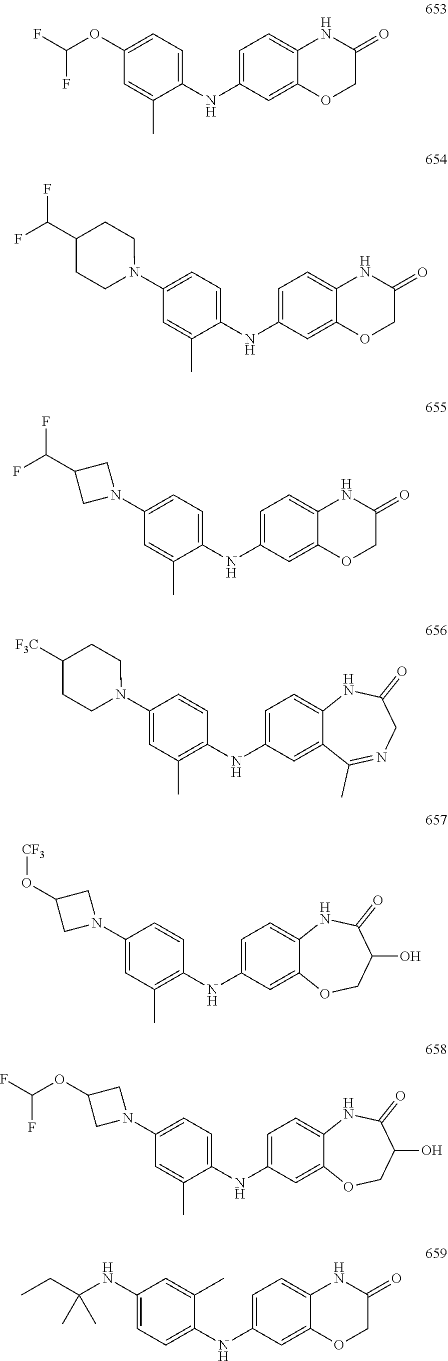
a tautomer thereof, a solvate or stereoisomer of the compound or the tautomer, or a pharmaceutically acceptable salt of the foregoing.

48. A pharmaceutical composition comprising a compound according to claim 1, a tautomer thereof, a solvate or stereoisomer of the compound or the tautomer, or a pharmaceutically acceptable salt of the foregoing and at least one pharmaceutically acceptable carrier.

49. A method of treating a disease or condition, comprising administering to a subject in need thereof, a therapeutically effective amount of a compound according to claim 1; wherein the disease or condition is selected from neuropathy, stroke, neurodegenerative disease, Parkinson's Disease, amyotrophic lateral sclerosis (AML), multiple sclerosis, Huntington's Disease, dementia with Lewy bodies, Friedreich's ataxia, hair follicle morphogenesis, diabetes, sepsis, transplant rejection, periventricular leukomalacia, ischemia reperfusion injury, blood coagulation, myocardial infarction, and kidney dysfunction.

50. A method of treating a disease or condition involving ferroptosis, comprising administering to a subject in need thereof, a therapeutically effective amount of a compound according to claim 1.

51. A method of modulating ferroptosis, comprising contacting a subject in need thereof with a compound according to claim 1.
